(19) United States
(12) Patent Application Publication
(10) Pub. No.: US 2010/0297109 A1
(43) Pub. Date: Nov. 25, 2010

(54) METHODS FOR INHIBITING FASCIN

(75) Inventor: Xin-Yun Huang, New York, NY (US)

Correspondence Address: FOLEY & LARDNER LLP, 150 EAST GILMAN STREET, P.O. BOX 1497, MADISON, WI, 53701-1497, US

(73) Assignee: Cornell University, Ithaca, NY (US)

(21) Appl. No.: 12/743,962

(22) PCT Filed: Nov. 21, 2008
(86) PCT No.: PCT/US08/12988

§ 371 Date: Jul. 29, 2010

Related U.S. Application Data
(60) Provisional application No. 60/989,609, filed on Nov. 21, 2007.

(51) Int. Cl. A61K 39/395; C12N 5/02; A61K 31/7088; A61K 38/16; A61P 35/00; A61P 25/00; A61P 31/12; A61P 31/04; G01N 33/566; C07H 21/04; C12N 15/63; C07K 16/00; C07K 14/435

(52) U.S. Cl. 424/130.1; 435/375; 514/44 A; 514/21.2; 514/19.3; 514/17.7; 514/3.7; 514/2.4; 514/19.4; 514/19.5; 436/501; 536/24.5; 435/320.1; 530/387.1; 530/350

(57) ABSTRACT

The invention relates to compositions and methods useful for inhibiting fascin. These compositions and methods can be used to inhibit fascin-related diseases. For example, according to the invention inhibition of fascin inhibits metastasis of tumor cells in mammals.

### RELATED APPLICATIONS

This application claims priority to the filing date of U.S. Provisional Application Ser. No. 60/989,609, filed Nov. 21, 2007, the contents of which are specifically incorporated by reference herein in their entirety.

This application is also related to U.S. application Ser. No. 10/551,152 filed Mar. 26, 2004, U.S. application Ser. No. 10/551,158 filed Mar. 26, 2004, PCT Application Ser. No. PCT/US04/09380 filed Mar. 26, 2004, U.S. Provisional Application No. 60/458,827, filed Mar. 28, 2003. The entire contents of each of the above-referenced applications are hereby specifically incorporated herein by reference in their entireties.

### GOVERNMENT FUNDING

The invention described in this application was made with funds from Department of Defense Grant Number BC050558. The United States government has certain rights in the invention.

### FIELD OF THE INVENTION

The invention relates to novel compositions and methods for inhibiting fascin expression and/or activity. According to the invention, such inhibition of fascin leads to inhibition of cell migration, including metastasis of cancer cells. The invention also relates to methods for identifying agents that modulate the expression and/or activity of fascin.

### BACKGROUND OF THE INVENTION

Despite the significant improvement in both diagnostic and therapeutic modalities for the treatment of cancer patients, tumor metastasis is still the major cause of mortality in cancer. Metastasis is the multi-step process wherein a primary tumor spreads from its initial site to secondary tissues/organs. This metastatic process is selective for cells that succeed in cell migration/invasion, embolization, survival in the circulation, arrest in a distant capillary bed, and extravasation into and multiplication within the organ parenchyma. Since tumor spreading is responsible for the majority of deaths of cancer patients, development of therapeutic agents that inhibit tumor metastasis is very desirable.

### SUMMARY OF THE INVENTION

The invention relates to methods of inhibiting fascin expression and/or activity. Fascin bundles F-actin polymers into highly dynamic membrane protrusions in motile cells. These actin-based, crosslinked protrusions support the outward extension of the leading edge of cellular mobility. As illustrated herein, knockdown of fascin expression in highly invasive breast tumor cells inhibits cell migration and invasion both in vitro and within in vivo animal models of metastatic cancer. The invention provides agents that modulate fascin expression and/or activity. Such agents are useful for treating and inhibiting diseases and conditions associated with fascin expression and/or activity, including metastatic cancer.

Therefore, one aspect of the invention is a method of inhibiting fascin expression and/or activity, comprising administering an effective amount of a fascin inhibitor to a cell expressing fascin to thereby inhibit the fascin expression or activity in the cell. For example, the fascin inhibitor can be an inhibitory nucleic acid that binds specifically to a fascin RNA or DNA consisting of SEQ ID NO:2, 4, 6 or 8, a small molecule, a fascin polypeptide fragment, or an antibody that binds specifically to fascin.

In some embodiments, the fascin inhibitor is an inhibitory nucleic acid that binds specifically to a fascin RNA or DNA consisting of SEQ ID NO:2, 4, 6 or 8.

The inhibitory nucleic acid can be an RNA or DNA, having a sequence that can be any of SEQ ID NOs:13-62, or a combination thereof. For example, the inhibitory nucleic acid can be administered by administering an expression vector that includes an expression cassette capable of directing the expression of the inhibitory nucleic acid.

The fascin inhibitor can also be an anti-fascin antibody. For example, the antibody can block actin binding to a fascin actin-binding site or can bind specifically to a fascin actin-binding site. In some embodiments, the fascin actin-binding site includes any of fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. In other embodiments, the fascin actin-binding site includes any of fascin amino acids His392, Glu391, Ala488, Lys471, His474 and Asp473. For example, the antibody can block actin binding to one or both of fascin amino acids His392 and His474 when bound to fascin protein. In other embodiments, the antibody can bind to one or both of fascin amino acids His392 and His474 when bound to fascin protein.

In some embodiments, the fascin inhibitor is a compound of formula I:

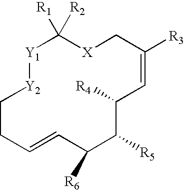

wherein:
X is CH, N, NH or O;
R₁ is OH, CZ₃ or R₁ and R₂ together are —C═O, wherein Z is halo;
R₂ is OH, CZ₃ or R₁ and R₂ together are —C═O, wherein Z is halo;
R₃ is H or lower alkyl;
R₄ is H or lower alkyl;
R₅ is OH;
R₆ is alkyloxy;
Y₁ and Y₂ are separately —CH₂— or Y₁ and Y₂ together form —C═C—

or a pharmaceutically acceptable salt thereof. Examples of compounds that can be used include any one of the following compounds, or a combination of such compounds:

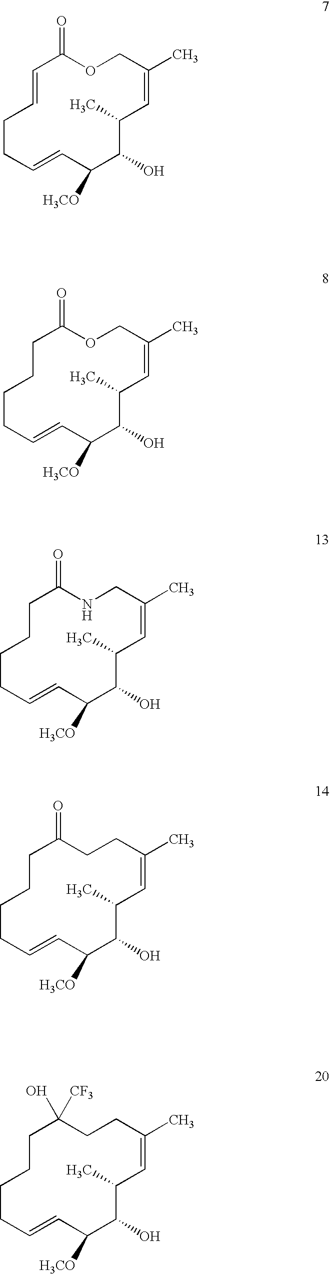

In some embodiments, the fascin inhibitor is not a migrastatin analog of formula I and is not compound 7, 8, 13, 14 or 20.

The cell is in an animal, for example, a human. Such an animal or human can be suffering from a disease or condition, for example, a disease involving expression or over-expression of fascin. The disease or condition can, for example, be a metastatic cancer, a neuronal disorder, neuronal degeneration, an inflammatory condition, a viral infection, a bacterial infection, lymphoid hyperplasia, Hodgkin's disease or ischemia-related tissue damage. In some embodiments, the cancer is a carcinoma, lymphoma, sarcoma, melanoma, astrocytoma, mesothelioma cells, ovarian carcinoma, colon carcinoma, pancreatic carcinoma, esophageal carcinoma, stomach carcinoma, lung carcinoma, urinary carcinoma, bladder carcinoma, breast cancer, gastric cancer, leukemia, lung cancer, colon cancer, central nervous system cancer, melanoma, ovarian cancer, renal cancer or prostate cancer.

Another aspect of the invention is a method of identifying an inhibitor of fascin, comprising: (a) contacting at least one protein or peptide having a fascin sequence with at least one test agent for a sufficient time to allow the components to interact; and (b) determining whether binding between the at least one protein or peptide having a fascin sequence and the test agent has occurred, wherein binding between the at least one protein or peptide having a fascin sequence and test agent is indicative that the test agent is an inhibitor of cancer metastasis. For example, the test agent can block actin binding to a fascin actin-binding site or binds to a fascin actin-binding site. The fascin actin-binding site can include fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. In other embodiments, the fascin actin-binding site can include fascin amino acids His392, Glu391, Ala488, Lys471, His474 and Asp473. For example, the test agent can block actin binding to one or both of fascin amino acids His392 and His474 when bound to fascin protein. In other embodiments, the test agent binds to one or both of fascin amino acids His392 and His474 when bound to fascin protein. The method can further include determining the binding constant of the test agent for fascin. The method can also determining whether the test agent inhibits fascin-mediated actin bundle formation. For example, the actin employed can be F-actin.

Another aspect of the invention is a method for identifying an inhibitor of fascin, comprising: (a) generating a three-dimensional structural image of a fascin binding site from fascin atomic coordinates for fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, according to Table 2, ± a root mean square deviation from the backbone atoms of said amino acids of not more than 1.5 angstroms; and (b) designing or selecting a potential inhibitor to reside within the fascin binding site to thereby identify an inhibitor of fascin.

Another aspect of the invention is a method for identifying an inhibitor of fascin, comprising: (a) generating a three-dimensional structural image of a fascin binding site from fascin atomic coordinates for fascin amino acids His392, Glu391, Ala488, Lys471, His474 and Asp473 according to Table 2, ± a root mean square deviation from the backbone atoms of said amino acids of not more than 1.5 angstroms; and (b) designing or selecting a potential inhibitor to reside within the fascin binding site to thereby identify an inhibitor of fascin.

Such methods can further include synthesizing or obtaining the potential inhibitor, contacting the potential inhibitor with fascin, and ascertaining whether the potential inhibitor binds to fascin. In some embodiments, the potential inhibitor is no larger than about eight (8) angstroms by about ten (10) angstroms by about ten (10) angstroms.

In some embodiments the method is performed using a computer system comprising the fascin atomic coordinates as a data set. The inhibitor of fascin that is identified can be an inhibitor of metastatic cancer.

Another aspect of the invention is a machine readable storage medium, comprising fascin atomic coordinates of Table 2. In some embodiments, the machine readable storage medium includes fascin atomic coordinates for fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, according to Table 2, ± a root mean square deviation from the backbone atoms of said amino acids of not more than 1.5 angstroms. In other embodiments, the machine readable storage medium includes fascin atomic coordinates for fascin amino acids His392, Glu391, Ala488, Lys471, His474 and Asp473 according to Table 2, ± a root mean square deviation from the backbone atoms of said amino acids of not more than 1.5 angstroms. Alternatively, the machine readable storage medium can include the atomic coordinates for both fascin actin sites.

Another aspect of the invention is a fascin inhibitor comprising an inhibitory nucleic acid that binds specifically to a fascin RNA or DNA consisting of SEQ ID NO:2, 4, 6 or 8, a small molecule, a fascin polypeptide fragment, or an antibody that binds specifically to fascin. For example, the inhibitory nucleic acid can be an RNA or DNA consisting of any of SEQ ID NOs:13-62. In some embodiments, the inhibitory nucleic acid is expressed in an expression vector comprising an expression cassette that directs the expression of a fascin inhibitory nucleic acid. The antibody can, for example, bind specifically to a fascin actin-binding site, or blocks actin-binding to a fascin actin-binding site, wherein the actin-binding site comprises fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. In other embodiments, the antibody can bind specifically to a fascin actin-binding site, or blocks actin-binding to a fascin actin-binding site, wherein the actin-binding site comprises fascin amino acids His392, Glu391, Ala488, Lys471, His474 and Asp473. For example, the antibody can be generated using a polypeptide with a sequence that includes fascin amino acids 259 through 493. Alternatively, for example, the antibody can be generated using a polypeptide with SEQ ID NO:9, 10 and/or 12. The fascin polypeptide fragment that is a fascin inhibit can include a peptide with fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. Alternatively, for example, the fascin polypeptide fragment that is a fascin inhibitor can include fascin amino acids His392, Glu391, Ala488, Lys471, His474 and Asp473. Thus, according to the invention, the fascin polypeptide fragment can consist of fascin amino acids 259 through 493, or a fascin polypeptide with SEQ ID NO:9, 10 and/or 12

Another aspect of the invention is a method of treating or inhibiting metastatic cancer in a patient, comprising administering to the patient, a fascin inhibitor of the invention.

Another aspect of the invention involves use of a fascin inhibitor in the manufacture of a medicament. For example, the medicament can be used for the treatment of metastatic cancer, a neuronal disorder, neuronal degeneration, an inflammatory condition, a viral infection, a bacterial infection, lymphoid hyperplasia, Hodgkin's disease or ischemia-related tissue damage. In some embodiments, the medicament is used for the treatment or inhibition of metastatic cancer or cancer cell in a mammal.

### BRIEF DESCRIPTION OF THE FIGURES

FIG. 1. Inhibition of mouse breast tumor 4T1 cell migration by core macroketone and core macrolactam. (A) Chemical structures of migrastatin, core macroketone, and core macrolactam. (B) Wound-healing assay showed that core macroketone (2 μM) and macrolactam (2 μM) inhibited the migration of mouse breast tumor 4T1 cells induced by serum. (C) Chamber assay of the effect of various concentrations of core macroketone on serum-induced 4T1 cell migration. (D) Chamber assay of the effect of core macrolactam on the serum-induced migration of 4T1 cells. Data represent mean±SD of three experiments.

FIG. 2. Inhibition of human tumor cell migration by core macroketone and core macrolactam. (A) Wound-healing assay showed that core macroketone (20 μM) and macrolactam (20 μM) inhibited the migration of human breast tumor MDA-MB-231 cells induced by serum. (B) IC₅₀ of core macroketone and core macrolactam on serum-induced migrations of human breast, colon, and prostate tumor cells. (C) Wound-healing assay showed that core macroketone (200 μM) and macrolactam (200 μM) had no effect on the migration of mouse embryonic fibroblast (MEF) cells induced by serum. (D) IC₅₀ of core macroketone and core macrolactam on serum (10% FBS)-induced migration of MEF and human mammary-gland epithelial MCF-10A cells and on N-formyl-peptide-induced (100 nM) migration of primary mouse leukocytes (neutrophils). Data are representative of three experiments.

FIG. 3. Inhibition of breast tumor metastasis by core macroketone and core macrolactam in a mouse model. (A) Lung metastasis was measured by the 6-thioguanine clonogenic assay. (B) Chemical structures of macrolactone and migrastatin semicore. (C) On the day the mice were killed, tumor diameter (in mm) was measured with an electronic caliper. Results are mean±SD (n=5). *, P<0.01.

FIG. 4. Identification of fascin as the macroketone binding protein. (A) Diagram of the structure of the biotin-conjugated macroketone core. (B) Coomassie staining of affinity purified proteins. Whole cell lysates from 200 plates (10 cm) of 4T1 cells were pre-cleared with Streptavidin beads. Half of the pre-cleared lysates were incubated with biotin-conjugated macroketone (lane 1); the other half with biotin (lane 2). After addition of Streptavidin beads, the solutions were transferred to Poly-Prep chromatography columns. After extensive washes, the bound proteins were eluted and analyzed on 10% SDS-PAGE. The arrow indicates the band identified as mouse fascin 1. Molecular mass markers are indicated on the left.

FIG. 5. Binding of purified fascin to macroketone. (A) Coomassie staining of purified GST-fascin (lane 2) and GST (lane 1) proteins. (B) Neutroavidin beads were mixed with biotin or biotin-macroketone. After wash, GST or GST-fascin was added. The reaction was incubated for 1 hour at room temperature. After wash with 300 mM NaCl, the samples were analyzed with SDS-PAGE. Lanes 5 and 6 were loaded with GST or GST-fascin proteins as controls. The top panel was probed with anti-GST antibody. The same filter was re-probed with anti-fascin antibody (the bottom panel). (C) Competition of unlabeled macroketone with biotin-conjugated macroketone to fascin. Increasing amounts of unlabeled macroketone (molar ratio of 1:1 and 10:1 of unlabeled macroketone over biotin-conjugated macroketone) decreased the binding of biotin-conjugated macroketone to fascin. Data are representative of three to five similar experiments.

FIG. 6. Macroketone inhibits the actin-bundling activity of fascin. (A)

Assay of the actin-bundling activity by the low-speed co-sedimentation assay. Polymerized F-actin (1 mM) was incubated with 0.125 μM or 0.25 μM purified fascin in the presence or absence of macroketone. Supernatants (S) or pellets (P) were analyzed by SDS-PAGE followed by Coomassie blue staining. A representative of five experiments with similar outcomes was shown. (B) Fluorescence microscopy of F-actin bundling. F-actin (1 mM) was incubated with fascin (0.125 μM) in the presence or absence of macroketone. Rhodamine-phalloidin was added to label actin filaments. Samples were mounted and imaged with a fluorescence microscopy. Left panel: in the absence of fascin, purified monomeric G-actin polymerized into F-actin, but without bundles. Middle panel: addition of purified fascin led to the bundling of actin polymers into thick filaments. Right panel: preincubation of fascin with macroketone decreased the ability of fascin to bundle actin polymers, thus leading to reduction of numbers of thick filaments. A representative of five experiments is shown. (C) Quantification of fluorescence microscopy-based F-actin bundling assays. Results are mean±SD (n=5, ·, p<0.05). (D) Electron microscopy of fascin-induced F-actin bundles in the presence or absence of macroketone. F-actin (1 mM) was incubated with fascin (0.125 μM) in the presence or absence of macroketone. Electron micrographs were obtained by negative staining of F-actin bundles. Representative images were shown. (E) Fascin and actin interaction assay. High-speed centrifugation was used to pellet F-actin polymers. Under these conditions, fascin alone was not precipitated and fascin could only be pulled-down by binding to F-actin polymers. While similar amounts of F-actin polymers were in the pellets in the absence and presence of macroketone (since the same amounts of F-actin polymers were added), significantly less fascin was pulled down by F-actin in the presence of macroketone

FIG. 7. Role of fascin in tumor cell migration. (A) Western blots showing that fascin siRNAs decreased the expression of fascin proteins. (B) Boyden chamber migration assay with 4T1 cells treated with control siRNA and two fascin siRNAs. Fascin siRNA treatment impaired the serum-induced migration of 4T1 cells. Results are mean±SD (n=5, p<0.05). (C) Boyden chamber migration assay of mouse fascin siRNA 2-treated 4T1 cells transfected with human wild-type GFP-fascin in the presence or absence of macroketone. Results are mean±SD (n=5, p<0.05). (D) Western blots show the expression levels of fascin protein in whole cell extracts prepared from 4T1 cells treated with control siRNA, fascin siRNA 2, or cells transfected with both mouse fascin siRNA 2 and human wild-type GFP-fascin. (E) Western blots show the expression of fascin protein in whole cell extracts prepared from human MDA-MB-231 cells treated with control siRNA and two different fascin siRNAs. (F) Boyden chamber migration assay with MDA-MB-231 cells treated with control siRNA and two fascin siRNAs. Fascin siRNA treatment impaired the serum-induced migration of MDA-MB-231 cells. Results are mean±SD (n=5, p<0.05). (G) Western blots show the expression of fascin protein in whole cell extracts prepared from non-invasive MCF-10A and metastatic MDA-MB-231 cells. (H) Chamber cell migration assay of MCF-10A cells transfected with control vector or GFP-fascin. Bottom panel shows the over-expression of fascin in MCF-10A cells. Results are mean±SD (n=5, p<0.05). (I) Chamber cell migration assay of mouse fascin siRNA 2-treated 4T1 cells transected with various mutants of GFP-human fascin (h-fascin) in the presence or absence of macroketone. Bottom panel shows the over-expression of various fascin mutants in mouse fascin siRNA 2-treated 4T1 cells. Results are mean±SD (n=5, p<0.05).

FIG. 8. Role of fascin in tumor metastasis. (A) In vitro Matrigel invasion assay with 4T1 cells treated with control siRNA and two different fascin siRNAs. Fascin siRNA treatment impaired serum-induced invasion of 4T1 cells. Results are mean±SD (n=5, p<0.05). (B) Primary mammary tumor growth of 4T1 cells expressing control siRNA and two fascin siRNAs. Results are mean±SD. (C) Primary mammary tumor weight four weeks after injecting 4T1 cells expressing control siRNA and two fascin siRNAs. (D) Total number of metastatic colonies in lungs of individual mice four weeks after injecting 4T1 cells expressing control siRNA and two fascin siRNAs. (E) Representative noninvasive bioluminescence images of mice at the indicated dates after injecting human MDA-MB-231 cells expressing control siRNA and two fascin siRNAs. (F) Normalized photon flux of noninvasive bioluminescence images of mice at the indicated dates after injecting human MDA-MB-231 cells expressing control siRNA and two fascin siRNAs. Results are mean±SD. (G) Histological analyses of the tumor tissues. Left panels, representative H&E staining of lungs from (E). Right panels, representative GFP imaging of lungs from (E). (H) Normalized photon flux of noninvasive bioluminescence images of mice at the indicated dates after injecting human MDA-MB-231 cells in the presence or absence of macroketone. Results are mean±SD.

FIG. 9. Elevated expression of fascin in human breast cancer patients. (A) Relative expression levels of fascin mRNA in normal and breast tumor samples. (B) Relative expression levels of fascin mRNA in normal breast tissue samples, Estrogen Receptor (ER)-positive breast tumors and ER-negative breast tumors. (C) Relative expression levels of fascin mRNA in normal breast tissue samples, Progesterone Receptor (PR)-positive breast tumors and PR-negative breast tumors. (D) Representative images of fascin immunohistological staining of ER-positive and ER-negative breast tumor samples. (E) Kaplan-Meier analysis of the probability of overall survival of patients with high fascin expression (log 10>0.1) or low fascin expression. (F) Kaplan-Meier analysis of the probability of metastasis-free survival of patients with high fascin expression (log 10>0.1) or low fascin expression. (G) Relative expression levels of fascin mRNA in the Rosetta microarray data set of ER-positive and ER-negative breast cancer samples. (H) Relative expression levels of fascin mRNA in the Rosetta microarray data set of PR-positive and PR-negative breast cancer samples.

FIG. 10. Overall structures of fascin and of the complex of fascin and macroketone. (A) Left panel, structure of fascin in the absence of macroketone shown as ribbon diagram, viewed from the N- and C-terminal plane. The four β-trefoil domains are colored red, yellow, green and blue. (B) Right panel, the structure in left panel turned 90° clockwise along the y-axis. (C) Overall structure of the complex of fascin and macroketone, with macroketone binding to the surface of trefoil-1.

FIG. 11. Macroketone binding site on fascin. (A) F_{obs}-F_{calc} map contoured at 3σ showing the macroketone binding site on fascin. (B) Molecular interactions between fascin residues and macroketone. (C) Superimposition of the α-carbon of fascin molecules in the absence (red) and the presence (black) of macroketone. (D) Local conformational changes induced by macroketone binding. The structure of fascin in the absence of macroketone is shown in gray. In the original, the structure of fascin with macroketone is shown in green, red and blue. Similarly, in the original, the structure of macroketone is shown in cyan and red.

FIG. 12. Actin binding sites on fascin. (A, B) Mutagenesis studies showed that both the N- and C-termini of fascin contribute to actin binding. (C) Residues from 29 to 42 are similar in sequences to an actin binding site on MARCKS. (D) Protein kinase C phosphorylation of Ser29 inhibited the actin bundling activity of fascin. (E) Genetic screening in Drosophila identified two mutations that reduced (mutation of Gly) or eliminated (mutation of Ser) the actin bundling activity of fascin. Since Ser274 is on the other side on this particular view of the model, residues (Gln277-Asp280) nearby Ser274 are shown to indicate the location.

FIG. 13. Macroketone binding site overlaps with one of the actin binding site. (A) Residues His392 and His474 involved in macroketone binding were shown in blue. Other residues involved in actin binding were shown in orange (the N-terminal domain), in light blue (Ser39), or in red (Gly393). (B) Based on the cryo-EM model of fimbrin and actin filaments, a model of fascin and two actin filaments are proposed.

FIG. 14. Mutagenesis studies of residues involved in macroketone binding and in actin bundling. (A) Coomassie blue stain of purified fascin and its mutant proteins. (B) Actin bundling assay for the wild-type fascin and its mutants. (C) Sensitivity to macroketone. Wild-type fascin, E391A and H474A mutants of fascin were assayed for their actin bundling activity in the absence or presence of macroketone.

FIG. 15 shows a diagram of a system used to carry out the instructions encoded by the storage medium of FIGS. 16 and 17.

FIG. 16 shows a cross section of a magnetic storage medium.

FIG. 17 shows a cross section of an optically-readable data storage medium.

### DETAILED DESCRIPTION OF THE INVENTION

The invention relates to compositions and methods for inhibiting fascin.

### Definitions

An “effective amount” generally means an amount which provides the desired effect. For example, an effective dose is an amount sufficient to effect a beneficial or desired result. The dose could be administered in one or more administrations. The precise determination of what would be considered an effective dose may be based on factors individual to each subject, including size, age, injury (e.g., defect) or disease (e.g., defect) being treated and amount of time since the injury occurred or the disease began. One skilled in the art, particularly a physician, would be able to determine the effective dose. Doses can vary depending on the mode of administration, e.g., local or systemic; free or encapsulated. The effect can be inhibition of metastasis or other clinical endpoints, such as treatment, reduction or regression of metastatic cancer. Other effects can include reduction or inhibition of fascin mRNA expression and/or protein levels.

A “cell that expresses fascin” or a “cell expressing fascin” is any human or animal cell that expresses fascin. In some embodiments, the cell over-expresses fascin. Such a cell can, for example, be a cancer cell, a neuron, an immune cell, or an antigen presenting cell. The cancer cell can be any cancer or tumor cell associated with the cancers or tumors described herein. For example, the cancer cell can be a cancerous breast, ovarian, colon, pancreatic, esophageal, stomach, lung, bladder, carcinoma, lymphoma, sarcoma, melanoma, or astrocytoma cell.

The term “actin-binding site” as used herein means a fascin peptide or fascin peptidomimetic that includes one of two sites where actin is bound by fascin. One fascin actin-binding site includes fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. The other fascin actin-binding site includes fascin amino acids His392, Glu391, Ala488, Lys471, His474 and Asp473.

The term “migrastatin analog binding site” as used herein means a fascin peptide or fascin peptidomimetic that includes the site where a migrstatin analog is bound by fascin. The mibrastatin binding site includes fascin amino acids His392, Glu391, Ala488, Lys471, His474 and Asp473. Actin can also bind to this site. While not wishing to be bound by any specific theory or mechanism, it is believed that migrastatin analogs inhibit the binding of actin to the migrastatin binding site.

The terms “small interfering RNA” or “siRNA” as used herein, refer to the mediators of RNAi, that is, RNA molecules capable of directing sequence-specific, post-transcriptional gene silencing of specific genes with which they share nucleotide sequence identity or similarity. In some organisms (e.g., C. elegans, D. melanogaster and various plants) these siRNAs can be created by the nucleolytic processing of longer dsRNAs. In mammalian cells they can also be produced from short (i.e., less than 30 base pairs) hairpin RNAs, or shRNAs.

The term “small hairpin siRNA,” “short hairpin siRNA,” or “shRNAs,” as used herein, refers to small interfering RNAs (siRNAs) composed of a single strand of RNA that possesses regions of self-complementarity that cause the single strand to fold back upon itself and form a hairpin-like structure with an intramolecular duplexed region containing at least 19 base pairs. Because they are single-stranded, shRNAs can be readily expressed from single expression cassettes.

The term “fascin inhibitor” as used herein means a siRNA or an antisense RNA capable of hybridizing or binding to a fascin nucleic acid (e.g., a fascin mRNA with any of SEQ ID NO: 2, 4, 6 or 8), a small molecule (e.g., a migrastatin analog), an anti-fascin antibody that binds specifically to fascin (e.g., to a fascin actin-binding site and/or to a fascin migrastatin binding site), a fascin peptide or fascin peptidomimetic that includes fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, a fascin peptide or fascin peptidomimetic that includes fascin amino acids His392, Glu391, Ala488, Lys471, His474 and Asp473.

The phrase “inhibiting fascin expression or activity” as used herein means suppressing the fascin gene expression, interfering with translation of the fascin gene product, interfering with the fascin gene product function (e.g., by reversibly or irreversibly binding an inhibitor or by blocking or disrupting fascin interaction with cellular products such as actin), inactivating the fascin gene product (e.g., by reaction with an inactivating agent), or removing the fascin gene product (e.g., by fascin gene mutation or by tagging the fascin gene product for cellular destruction).

The term “knock down,” as used herein, describes the condition where expression of a gene is reduced. For example, “knock down” can be created by mutation of a gene, deletion of a gene, or reduction in expression of a gene. One method for reducing expression of a gene involves RNAi, wherein the expression of a particular gene-product, or the cellular concentration of a particular RNA transcript, is reduced or eliminated by the sequence-specific, post-transcriptional gene silencing initiated by siRNAs that are homologous in sequence to the gene encoding said gene product. Hence, as used herein RNAi is a “knock down” agent.

A “subject” is a vertebrate, preferably a mammal, more preferably a human. Mammals include, but are not limited to, humans, farm animals, sport animals and pets. Included in the terms animals or pets are, but not limited to, dogs, cats, horses, rabbits, mice, rats, sheep, goats, cows and birds.

As used herein, “treat,” “treating” or “treatment” includes treating, reversing, preventing, reducing, ameliorating, or inhibiting an injury or disease-related condition or a symptom of an injury or disease-related condition.

The terms “comprises”, “comprising”, and the like can have the meaning ascribed to them in U.S. Patent Law and can mean “includes”, “including” and the like. As used herein, “including” or “includes” or the like means including, without limitation.

### Fascin

Fascin is an actin-bundling protein that has a major function in forming parallel actin bundles in cell protrusions such as lamellipodia, which are key specializations of the plasma membrane for cell migration (Adams 2004). Fascin mRNA is not usually expressed by normal epithelial cells, but its overexpression has been reported in many different types of carcinomas, including breast, ovary, colon, pancreas, esophagus, stomach, lung, and urinary bladder, as well as in other tumors, such as lymphomas, sarcomas, melanomas, and astrocytomas. The high expression of fascin mRNA is correlated with an aggressive clinical course and shorter survival. Fascin has been identified as the protein target of the migrastatin analogs described herein.

Fascin organizes actin into highly dynamic and architecturally diverse subcellular scaffolds. These scaffolds orchestrate a variety of mechanical processes, including filopodial protrusions in motile cells.

Sequences for fascin from a variety of sources are available. For example, publicly accessible databases of amino acid and nucleic acid sequences can be searched for fascin sequences. One example of a sequence for human fascin can be found in the database maintained by the National Center of Biotechnology Information at the www.ncbi.nlm.nih.gov website (accession number AAL01526, gi: 15625241), which is provided below as SEQ ID NO:1 for easy reference.

| | |
|---|---|
| 1 | MTANGTAEAV QIQFGLINCG NKYLTAEAFG FKVNASASSL |
| 41 | KKKQIWTLEQ PPDEAGSAAV CLRSHLGRYL AADKDGNVTC |
| 81 | EREVPGPDCR FLIVAHDDGR WSLQSEAHRR YFGGTEDRLS |
| 121 | CFAQTVSPAE KWSVHIAMHP QVNIYSVTRK RYAHLSARPA |
| 161 | DEIAVDRDVP WGVDSLITLA FQDQRYSVQT ADHRFLRHDG |
| 201 | RLVARPEPAT GYTLEFRSGK VAFRDCEGRY LAPSGPSGTL |
| 241 | KAGKATKVGK DELFALEQSC AQVVLQAANE RNVSTRQGMD |
| 281 | LSANQDEETD QETFQLEIDR DTKKCAFRTH TGKYWTLTAT |
| 321 | GGVQSTASSK NASCYFDIEW RDRRITLRAS NGKFVTSKKN |
| 361 | GQLAASVETA GDSELFLMKL INRPIIVFRG EHGFIGCRKV |
| 401 | TGTLDANRSS YDVFQLEFND GAYNIKDSTG KYWTVGSDSA |
| 441 | VTSSGDTPVD FFFEFCDYNK VAIKVGGRYL KGDHAGVLKA |
| 481 | SAETVDPASL WEY |

A genomic nucleotide sequence for the SEQ ID NO:1 fascin polypeptide is found, for example, at NCBI accession no. AY044229, gi: 15625240. A cDNA sequence for the SEQ ID NO:1 polypeptide can be found in the NCBI database as accession no. BC006304 (gi: 33873525). This nucleotide sequence is provided below for easy reference as SEQ ID NO:2.

| | |
|---|---|
| 1 | GCTGCGGAGG GTGCGTGCGG GCCGCGGCAG CCGAACAAAG |
| 41 | GAGCAGGGGC GCCGCCGCAG GGACCCGCCA CCCACCTCCC |
| 81 | GGGGCCGCGC AGCGGCCTCT CGTCTACTGC CACCATGACC |
| 121 | GCCAACGGCA CAGCCGAGGC GGTGCAGATC CAGTTCGGCC |
| 161 | TCATCAACTG CGGCAACAAG TACCTGACGG CCGAGGCGTT |
| 201 | CGGGTTCAAG GTGAACGCGT CCGCCAGCAG CCTGAAGAAG |
| 241 | AAGCAGATCT GGACGCTGGA GCAGCCCCCT GACGAGGCGG |
| 281 | GCAGCGCGGC CGTGTGCCTG CGCAGCCACC TGGGCCGCTA |
| 321 | CCTGGCGGCG GACAAGGACG GCAACGTGAC CTGCGAGCGC |
| 361 | GAGGTGCCCG GTCCCGACTG CCGTTTCCTC ATCGTGGCGC |
| 401 | ACGACGACGG TCGCTGGTCG CTGCAGTCCG AGGCGCACCG |
| 441 | GCGCTACTTC GGCGGCACCG AGGACCGCCT GTCCTGCTTC |
| 481 | GCGCAGACGG TGTCCCCCGC CGAGAAGTGG AGCGTGCACA |
| 521 | TCGCCATGCA CCCTCAGGTC AACATCTACA GCGTCACCCG |
| 561 | TAAGCGCTAC GCGCACCTGA GCGCGCGGCC GGCCGACGAG |
| 601 | ATCGCCGTGG ACCGCGACGT GCCCTGGGGC GTCGACTCGC |
| 641 | TCATCACCCT CGCCTTCCAG GACCAGCGCT ACAGCGTGCA |
| 681 | GACCGCCGAC CACCGCTTCC TGCGCCACGA CGGGCGCCTG |
| 721 | GTGGCGCGCC CCGAGCCGGC CACTGGCTAC ACGCTGGAGT |
| 761 | TCCGCTCCGG CAAGGTGGCC TTCCGCGACT GCGAGGGCCG |
| 801 | TTACCTGGCG CCGTCGGGGC CCAGCGGCAC GCTCAAGGCG |
| 841 | GGCAAGGCCA CCAAGGTGGG CAAGGACGAG CTCTTTGCTC |
| 881 | TGGAGCAGAG CTGCGCCCAG GTCGTGCTGC AGGCGGCCAA |
| 921 | CGAGAGGAAC GTGTCCACGC GCCAGGGTAT GGACCTGTCT |
| 961 | GCCAATCAGG ACGAGGAGAC CGACCAGGAG ACCTTCCAGC |
| 1001 | TGGAGATCGA CCGCGACACC AAAAAGTGTG CCTTCCGTAC |
| 1041 | CCACACGGGC AAGTACTGGA CGCTGACGGC CACCGGGGGC |
| 1081 | GTGCAGTCCA CCGCCTCCAG CAAGAATGCC AGCTGCTACT |
| 1121 | TTGACATCGA GTGGCGTGAC CGGCGCATCA CACTGAGGGC |
| 1161 | GTCCAATGGC AAGTTTGTGA CCTCCAAGAA GAATGGGCAG |
| 1201 | CTGGCCGCCT CGGTGGAGAC AGCAGGGGAC TCAGAGCTCT |
| 1241 | TCCTCATGAA GCTCATCAAC CGCCCCATCA TCGTGTTCCG |
| 1281 | CGGGGAGCAT GGCTTCATCG GCTGCCGCAA GGTCACGGGC |
| 1321 | ACCCTGGACG CCAACCGCTC CAGCTATGAC GTCTTCCAGC |
| 1361 | TGGAGTTCAA CGATGGCGCC TACAACATCA AAGACTCCAC |
| 1401 | AGGCAAATAC TGGACGGTGG GCAGTGACTC CGTGGTCACC |
| 1441 | AGCAGCGGCG ACACTCCTGT GGACTTCTTC TTCGAGTTCT |
| 1481 | GCGACTATAA CAAGGTGGCC ATCAAGGTGG GCGGGCGCTA |
| 1521 | CCTGAAGGGC GACCACGCAG GCGTCCTGAA GGCCTCGGCG |
| 1561 | GAAACCGTGG ACCCCGCCTC GCTCTGGGAG TACTAGGGCC |
| 1601 | GGCCCGTCCT TCCCCGCCCC TGCCCACATG GCGGCTCCTG |
| 1641 | CCAACCCTCC CTGCTAACCC CTTCTCCGCC AGGTGGGCTC |
| 1681 | CAGGGCGGGA GGCAAGCCCC CTTGCCTTTC AAACTGGAAA |
| 1721 | CCCCAGAGAA AACGGTGCCC CCACCTGTCG CCCCTATGGA |
| 1761 | CTCCCCACTC TCCCCTCCGC CCGGGTTCCC TACTCCCCTC |
| 1801 | GGGTCAGCGG CTGCGGCCTG GCCCTGGGAG GGATTTCAGA |
| 1841 | TGCCCCTGCC CTCTTGTCTG CCACGGGGCG AGTCTGGCAC |
| 1881 | CTCTTTCTTC TGACCTCAGA CGGCTCTGAG CCTTATTTCT |
| 1921 | CTGGAAGCGG CTAAGGGACG GTTGGGGGCT GGGAGCCCTG |
| 1961 | GGCGTGTAGT GTAACTGGAA TCTTTTGCCT CTCCCAGCCA |
| 2001 | CCTCCTCCCA GCCCCCCAGG AGAGCTGGGC ACATGTCCCA |
| 2041 | AGCCTGTCAG TGGCCCTCCC TGGTGCACTG TCCCCGAAAC |
| 2081 | CCCTGCTTGG GAAGGGAAGC TGTCGGGTGG GCTAGGACTG |
| 2121 | ACCCTTGTGG TGTTTTTTTG GGTGGTGGCT GGAAACAGCC |
| 2161 | CCTCTCCCAC GTGGCAGAGG CTCAGCCTGG CTCCCTTCCC |
| 2201 | TGGAGCGGCA GGGCGTGACG GCCACAGGGT CTGCCCGCTG |
| 2241 | CACGTTCTGC CAAGGTGGTG GTGGCGGGCG GGTAGGGGTG |
| 2281 | TGGGGGCCGT CTTCCTCCTG TCTCTTTCCT TTCACCCTAG |
| 2321 | CCTGACTGGA AGCAGAAAAT GACCAAATCA GTATTTTTTT |
| 2361 | TAATGAAATA TTATTGCTGG AGGCGTCCCA GGCAAGCCTG |
| 2401 | GCTGTAGTAG CGAGTGATCT GGCGGGGGGC GTCTCAGCAC |
| 2441 | CCTCCCCAGG GGGTGCATCT CAGCCCCCTC TTTCCGTCCT |
| 2481 | TCCCGTCCAG CCCCAGCCCT GGGCCTGGGC TGCCGACACC |
| 2521 | TGGGCCAGAG CCCCTGCTGT GATTGGTGCT CCCTGGGCCT |
| 2561 | CCCGGGTGGA TGAAGCCAGG CGTCGCCCCC TCCGGGAGCC |
| 2601 | CTGGGGTGAG CCGCCGGGGC CCCCCCTGCT GCCAGCCTCC |
| 2641 | CCCGTCCCCA ACATGCATCT CACTCTGGGT GTCTTGGTCT |
| 2681 | TTTATTTTTT GTAAGTGTCA TTTGTATAAC TCTAAACGCC |
| 2721 | CATGATAGTA GCTTCAAACT GGAAATAGCG AAATAAAATA |
| 2761 | ACTCAGTCTG CAGCCCCAAA AAAAAAAAAA AAAAAAAAAA |

One example of a sequence for human fascin 2 (accession no. NP_{—}001070650, gi: 116295251) is provided below as SEQ ID NO:3:

| | |
|---|---|
| 1 | MPTNGLHQVL KIQFGLVNDT DRYLTAESFG FKVNASAPSL |
| 41 | KRKQTWVLEP DPGQGTAVLL RSSHLGRYLS AEEDGRVACE |
| 81 | AEQPGRDCRF LVLPQPDGRW VLRSEPHGRF FGGTEDQLSC |
| 121 | FATAVSPAEL WTVHLAIHPQ AHLLSVSRRR YVHLCPREDE |
| 161 | MAADGDKPWG VDALLTLIFR SRRYCLKSCD SRYLRSDGRL |
| 201 | VWEPEPRACY TLEFKAGKLA FKDCDGHYLA PVGPAGTLKA |
| 241 | GRNTRPGKDE LFDLEESHPQ VVLVAANHRY VSVRQGVNVS |
| 281 | ANQDDELDHE TFLMQIDQET KKCTFYSSTG GYWTLVTHGG |
| 321 | IHATATQVSA NTMFEMEWRG RRVALKASNG RYVCMKKNGQ |
| 361 | LAAISDFVGP PPRPAWTGKV AGGAAQQTLS PPGKDEEFTL |
| 401 | KLINRPILVL RGLDGFVCHH RGSNQLDTNR SVYDVFHLSF |
| 441 | SDGAYRIRGR DGGFWYTGSH GSVCSDGERA EDFVFEFRER |
| 481 | GRLAIRARSG KYLRGGASGL LRADADAPAG TALWEY |

A cDNA sequence for the SEQ ID NO:3 polypeptide can be found in the NCBI database as accession no. NM001077182 (gi: 116295250). This nucleotide sequence is provided below for easy reference as SEQ ID NO:4.

| | |
|---|---|
| 1 | GCAGGCAGGG GGTTCGTGAC GCCGGCTGGG TCTGGGGGCT |
| 41 | GTGGGCCAGC CGAGCCGACC CGGGCTTCTG GGGGACCGCG |
| 81 | GGGGCCGTGA GCACTCAGAG GGTGCATCCC AGGCCCCTCC |
| 121 | GGGGACCCGG CCAGCCTGAA GATGCCGACG AACGGCCTGC |
| 161 | ACCAGGTGCT GAAGATCCAG TTTGGCCTCG TCAACGACAC |
| 201 | TGACCGCTAC CTGACAGCTG AGAGCTTCGG CTTCAAGGTC |
| 241 | AATGCCTCGG CACCCAGCCT CAAGAGGAAG CAGACCTGGG |
| 281 | TGCTGGAACC CGACCCAGGA CAAGGCACGG CTGTGCTGCT |
| 321 | CCGCAGCAGC CACCTGGGCC GCTACCTGTC GGCAGAAGAG |
| 361 | GACGGGCGCG TGGCCTGTGA GGCAGAGCAG CCGGGCCGTG |
| 401 | ACTGCCGCTT CCTGGTCCTG CCGCAGCCAG ATGGGCGCTG |
| 441 | GGTGCTGCGG TCCGAGCCGC ACGGCCGCTT CTTCGGAGGC |
| 481 | ACCGAGGACC AGCTGTCCTG CTTCGCCACA GCCGTTTCCC |
| 521 | CGGCCGAGCT GTGGACCGTG CACCTGGCCA TCCACCCGCA |
| 561 | GGCCCACCTG CTGAGCGTGA GCCGGCGGCG CTACGTGCAC |
| 601 | CTGTGCCCGC GGGAGGACGA GATGGCCGCA GACGGAGACA |
| 641 | AGCCCTGGGG CGTGGACGCC CTCCTCACCC TCATCTTCCG |
| 681 | GAGCCGACGG TACTGCCTCA AGTCCTGTGA CAGCCGCTAC |
| 721 | CTGCGCAGCG ACGGCCGTCT GGTCTGGGAG CCTGAGCCCC |
| 761 | GTGCCTGCTA CACGCTGGAG TTCAAGGCGG GCAAGCTGGC |
| 801 | CTTCAAGGAC TGCGACGGCC ACTACCTGGC ACCCGTGGGG |
| 841 | CCCGCAGGCA CCCTCAAGGC CGGCCGAAAC ACGCGACCTG |
| 881 | GCAAGGATGA GCTCTTTGAT CTGGAGGAGA GTCACCCACA |
| 921 | GGTGGTGCTG GTGGCTGCCA ACCACCGCTA CGTCTCTGTG |
| 961 | CGGCAAGGGG TCAACGTCTC AGCCAATCAG GATGATGAAC |
| 1001 | TAGACCACGA GACCTTCCTG ATGCAAATTG ACCAGGAGAC |
| 1041 | AAAGAAGTGC ACCTTCTATT CCAGCACTGG GGGCTACTGG |
| 1081 | ACCCTGGTCA CCCATGGGGG CATTCACGCC ACAGCCACAC |
| 1121 | AAGTTTCTGC CAACACCATG TTTGAGATGG AGTGGCGTGG |
| 1161 | CCGGCGGGTA GCACTCAAAG CCAGCACTGG GCGCTACGTG |
| 1201 | TGCATGAAGA AGAATGGGCA GCTGGCGGCT ATCAGCGATT |
| 1241 | TTGTCGGGCC CCCACCCCGC CCGGCCTGGA CAGGGAAGGT |
| 1281 | GGCGGGAGGG GCAGCGCAGC AGACGCTCTC CCCGCCAGGC |
| 1321 | AAGGACGAAG AGTTCACCCT CAAGCTCATC AACCGGCCCA |
| 1361 | TCCTGGTGCT GCGCGGCCTG GACGGCTTCG TCTGCCACCA |
| 1401 | CCGCGGCTCC AACCAGCTGG ACACCAACCG CTCCGTCTAC |
| 1441 | GACGTCTTCC ACCTGAGCTT CAGCGACGGC GCCTACCGGA |
| 1481 | TCCGAGGCCG CGACGGAGGG TTCTGGTACA CGGGCAGCCA |
| 1521 | CGGCAGCGTG TGCAGCGACG GCGAACGCGC CGAGGACTTC |
| 1561 | GTCTTCGAGT TCCGTGAGCG CGGCCGCCTG GCCATCCGCG |
| 1601 | CCCGGAGCGG CAAGTACCTG CGCGGCGGCG CCTCGGGCCT |
| 1641 | GCTGCGGGCC GATGCCGACG CCCCGGCCGG GACCGCGCTT |
| 1681 | TGGGAGTACT GAGGCCGCGC CCAGACCAGC CTGTCGCGCA |
| 1721 | TTAAAACCGT GTCTCTCCCG CAAAAAAAAA AAAAAAAAAA |
| 1761 | AA |

One example of a sequence for human fascin 3 (accession no. NP_{—}065102, gi: 9966791) is provided below as SEQ ID NO:5:

| | |
|---|---|
| 1 | MDETEWIHRH PKAEDLRVGL ISWAGTYLTF EACKNTVTAT |
| 41 | AKSLGRRQTW EILVSNEHET QAVVRLKSVQ GLYLLCECDG |
| 81 | TVCYGRPRTS HHGCFLLRFH RNSKWTLQCL ISGRYLESNG |
| 121 | KDVFCTSHVL SAYHMWTPRP ALHVHVILYS PIHRCYARAD |
| 161 | PTMGRIWVDA AVPCLEECGF LLHFRDGCYH LETSTHHFLS |
| 201 | HVDRLFSQPS SQTAFHMQVR PGGLVALCDG EGGMLYPQGT |
| 241 | HLLLGMGCNP MRGEEWFILQ HCPTWVSLRS KTGRFISVIY |
| 281 | DGEVRAASER LNRMSLFQFE CDSESPTVQL RSANGYYLSQ |
| 321 | RRHRAVMADG HPLESDTFFR MHWNCGRIIL QSCRGRFLGI |
| 361 | APNSLLMANV ILPGPNEEFG ILFANRSFLV LRGRYGYVGS |
| 401 | SSGHDLIQCN QDQPDRIHLL PCRPGIYHFQ AQGGSFWSIT |
| 441 | SFGTFRPWGK FALNFCIELQ GSNLLTVLAP NGFYMRADQS |
| 481 | GTLLADSEDI TRECIWEF |

A cDNA sequence for the SEQ ID NO:5 fascin polypeptide can be found in the NCBI database as accession no. NM_{—}020369 (gi: 9966790). This nucleotide sequence is provided below for easy reference as SEQ ID NO:6.

| | |
|---|---|
| 1 | CCCTTTCCCC ACTGTGGTGT GATAAGAGGC TGCCCTCACA |
| 41 | GTCACAATGC TCCCGGGTCA CAGAGGTGCT GGGCCCCAGG |
| 81 | CCAGCCTCTG CCTGGGAAGT TCTCTCTGGG AACATCTGGT |
| 121 | GGGTACTACA GGCCCTATTC CAGGCCCTAT GGCCTGTGGA |
| 161 | ACCTCACCAC GGGGGGGAGG GCTGGGCCAG ACGGAGACAT |
| 201 | CACCTGTGGT GTCAGCCCCA TGGATGAGAC AGAGTGGATA |
| 241 | CACAGACATC CCAAGGCTGA GGACCTAAGG GTTGGGCTCA |
| 281 | TCAGCTGGGC AGGAACCTAC CTCACCTTTG AGGCATGCAA |
| 321 | GAATACAGTC ACTGCAACTG CGAAGAGTTT GGGCAGGAGA |
| 361 | CAGACCTGGG AGATCTTGGT GAGCAATGAG CATGAGACAC |
| 401 | AGGCCGTGGT GCGACTAAAG AGCGTGCAGG GCCTCTACCT |
| 441 | GCTGTGTGAG TGTGATGGCA CCGTGTGTTA TGGCCGCCCA |
| 481 | AGGACCAGCC ACCATGGGTG CTTTCTACTG CGTTTCCACC |
| 521 | GGAACAGCAA GTGGACCCTC CAGTGCCTAA TCTCTGGTCG |
| 561 | TTATTTGGAG TCCAATGGCA AGGACGTGTT TTGCACTTCC |
| 601 | CACGTCCTCT CAGCTTACCA CATGTGGACC CCCCGACCAG |
| 641 | CCCTCCATGT CCACGTGATC CTCTACAGCC CCATCCACCG |
| 681 | CTGCTATGCC CGGGCTGACC CCACTATGGG CCGCATCTGG |
| 721 | GTGGACGCAG CAGTTCCCTG CCTGGAGGAG TGTGGCTTCC |
| 761 | TGTTGCATTT CCGAGATGGA TGCTACCACC TGGAGACCTC |
| 801 | TACACACCAC TTCTTGTCCC ATGTAGACCG GCTGTTCTCC |
| 841 | CAACCCTCAT CACAGACAGC TTTTCACATG CAAGTGCGGC |
| 881 | CTGGAGGGCT TGTGGCACTG TGTGATGGAG AAGGAGGCAT |
| 921 | GTTATATCCA CAGGGCACGC ATCTGCTCTT GGGCATGGGC |
| 961 | TGCAACCCCA TGAGGGGTGA GGAGTGGTTC ATCCTACAGC |
| 1001 | ACTGCCCAAC CTGGGTCAGC CTCAGGTCAA AGACTGGGCG |
| 1041 | GTTCATCTCA GTCATCTACG ATGGTGAGGT GCGTGCTGCT |
| 1081 | TCTGAGCGCT TAAACCGAAT GTCCTTGTTC CAGTTTGAAT |
| 1121 | GTGACAGTGA GAGCCCCACT GTGCAGCTTC GTTCAGCCAA |
| 1161 | TGGCTACTAC CTATCCCAGA GGCGCCACAG GGCAGTAATG |
| 1201 | GCTGATGGGC ACCCCCTGGA GTCTGACACG TTCTTCCGAA |
| 1241 | TGCACTGGAA CTGTGGCAGG ATCATCCTGC AGTCCTGCAG |
| 1281 | GGGGCGCTTC CTGGGCATTG CACCCAACAG CCTGCTGATG |
| 1321 | GCCAATGTCA TCCTTCCAGG CCCAAATGAG GAATTTGGGA |
| 1361 | TTTTATTTGC CAATCGCTCC TTCCTTGTAT TGCGAGGTCG |
| 1401 | TTATGGCTAT GTGGGCTCCT CATCGGGCCA TGACCTCATA |
| 1441 | CAGTGCAACC AGGATCAGCC CGACCGCATT CATCTACTAC |
| 1481 | CCTGCCGACC GGGTATCTAC CACTTCCAGG CACAGGGGGG |
| 1521 | ATCCTTCTGG TCAATAACAT CCTTTGGCAC CTTTCGCCCT |
| 1561 | TGGGGCAAGT TTGCCCTCAA CTTCTGTATC GAGCTTCAGG |
| 1601 | GGAGCAACTT ACTCACTGTA CTGGCCCCCA ATGGCTTCTA |
| 1641 | CATGCGAGCC GACCAAAGTG GCACCCTGTT GGCAGACAGT |
| 1681 | GAAGACATTA CCAGAGAGTG TATCTGGGAA TTTTAGGTCA |
| 1721 | ATGGGATGTC ACCTACCAAA ATCCAAATCC TCCAGGAAAA |
| 1761 | ACTACTACAC TAAATGGACC AGGAACCTCA GAGTCAAGAT |
| 1801 | CCAAGAGAAG AACATCTGTT ACAACTTTTC CTACCCAGTT |
| 1841 | TAGCAAAACA CCTGTTTTAT GCAACAATAC ATCACAACAG |
| 1881 | GCC |

One example of a sequence for mouse fascin homolog 1 (accession number NP 032010, gi: 113680348) is provided below as SEQ ID NO:7:

| | |
|---|---|
| 1 | MTANGTAEAV QIQFGLISCG NKYLTAEAFG FKVNASASSL |
| 41 | KKKQIWTLEQ PPDEAGSAAV CLRSHLGRYL AADKDGNVTC |
| 81 | EREVPDGDCR FLVVAHDDGR WSLQSEAHRR YFGGTEDRLS |
| 121 | CFAQSVSPAE KWSVHIAMHP QVNIYSVTRK RYAHLSARPA |
| 161 | DEIAVDRDVP WGVDSLITLA FQDQRYSVQT SDHRFLRHDG |
| 201 | RLVARPEPAT GFTLEFRSGK VAFRDCEGRY LAPSGPSGTL |
| 241 | KAGKATKVGK DELFALEQSC AQVVLQAANE RNVSTRQGMD |
| 281 | LSANQDEETD QETFQLEIDR DTRKCAFRTH TGKYWTLTAT |
| 321 | GGVQSTASTK NASCYFDIEW CDRRITLRAS NGKFVTAKKN |
| 361 | GQLAASVETA GDSELFLMKL INRPIIVFRG EHGFIGCRKV |
| 401 | TGTLDANRSS YDVFQLEFND GAYNIKDSTG KYWTVGSDSS |
| 441 | VTSSSDTPVD FFLEFCDYNK VALKVGGRYL KGDHAGVLKA |
| 481 | CAETIDPASL WEY |

A cDNA sequence for the SEQ ID NO:7 fascin polypeptide can be found in the NCBI database as accession no. NM_{—}007984 (gi: 113680347). This nucleotide sequence is provided below for easy reference as SEQ ID NO:8.

| | |
|---|---|
| 1 | TGTGGAGAAC TGCAGCGGGC TAAGCCGTGT TGAACAAAGG |
| 41 | AGGTCGGGCA CAGCTATCCA AGCTCCCGGG GCCACCGGGC |
| 81 | CGCCCTCCGC CACCATGACC GCCAACGGCA CGGCAGAGGC |
| 121 | TGTGCAGATT CAGTTCGGGC TCATCAGCTG CGGCAACAAG |
| 161 | TACCTGACAG CCGAGGCGTT CGGGTTCAAG GTGAACGCAT |
| 201 | CCGCTAGTAG CTTGAAAAAG AAGCAGATCT GGACGCTGGA |
| 241 | GCAACCTCCC GATGAGGCGG GCAGCGCGGC CGTGTGTCTG |
| 281 | CGCAGCCACC TGGGTCGCTA CCTGGCCGCC GACAAGGACG |
| 321 | GCAACGTGAC CTGCGAGCGC GAGGTGCCCG ACGGCGACTG |
| 361 | CCGCTTTCTC GTCGTGGCGC ACGACGACGG CCGCTGGTCG |
| 401 | CTGCAGTCCG AGGCTCACCG GCGCTACTTT GGCGGCACCG |
| 441 | AGGACCGCCT GTCCTGCTTC GCGCAGAGCG TGTCGCCGGC |
| 481 | CGAGAAGTGG AGCGTGCACA TCGCCATGCA CCCGCAGGTT |
| 521 | AACATCTACA GCGTTACCCG CAAGCGCTAC GCGCATCTGA |
| 561 | GCGCGCGGCC GGCCGACGAG ATCGCGGTAG ACCGCGACGT |
| 601 | GCCTTGGGGC GTCGACTCGC TCATCACCTT GGCCTTCCAG |
| 641 | GACCAACGCT ACAGTGTGCA GACGTCCGAC CACCGCTTCC |
| 681 | TGCGCCACGA CGGGCGCCTT GTGGCACGGC CGGAGCCCGC |
| 721 | CACGGGCTTC ACGCTGGAGT TCCGCTCCGG CAAGGTGGCC |
| 761 | TTTCGCGACT GCGAAGGTCG CTACCTGGCT CCGTCCGGGC |
| 801 | CCAGCGGCAC CCTCAAGGCT GGCAAGGCCA CCAAGGTGGG |
| 841 | CAAAGATGAG CTCTTCGCCC TGGAACAGAG CTGCGCTCAG |
| 881 | GTGGTGCTGC AGGCGGCCAA CGAGAGGAAC GTGTCCACGC |
| 921 | GCCAGGGAAT GGACCTGTCA GCCAATCAGG ATGAAGAGAC |
| 961 | CGATCAGGAG ACCTTCCAGC TGGAGATCGA CCGCGACACA |
| 1001 | AGAAAGTGTG CCTTTCGCAC CCACACGGGC AAGTACTGGA |
| 1041 | CACTGACGGC GACCGGAGGT GTGCAATCCA CTGCGTCCAC |
| 1081 | CAAGAACGCC AGCTGCTACT TTGACATCGA GTGGTGTGAC |
| 1121 | CGCCGGATCA CTCTGAGAGC CTCCAACGGC AAGTTTGTGA |
| 1161 | CCGCCAAGAA AAATGGCCAG CTGGCCGCCT CGGTGGAGAC |
| 1201 | AGCAGGGGAC TCGGAACTCT TCCTCATGAA GCTGATTAAC |
| 1241 | CGCCCCATCA TCGTGTTCCG GGGGGAACAC GGGTTCATTG |
| 1281 | GCTGCCGCAA GGTCACGGGC ACTCTGGATG CCAACCGTTC |
| 1321 | CAGTTACGAT GTCTTCCAGT TGGAATTCAA TGACGGCGCC |
| 1361 | TACAACATCA AAGACTCCAC GGGCAAGTAC TGGACGGTGG |
| 1401 | GTAGTGATTC CTCGGTCACC AGCAGCAGCG ACACCCCTGT |
| 1441 | GGATTTCTTC CTTGAGTTCT GTGACTACAA TAAGGTGGCT |
| 1481 | CTCAAGGTGG GCGGCCGCTA CCTGAAGGGG GACCACGCTG |
| 1521 | GGGTCCTGAA GGCCTGCGCG GAGACTATCG ACCCCGCCTC |
| 1561 | ACTCTGGGAG TACTAGGGCC ACCTGCCCTC TGCAGGCCGC |
| 1601 | TCTCGTCAGT CCCTCCTGTT ATCCTTACTC ATCGGGTGGC |
| 1641 | CCTGCAGCAG GTGGCAAACC CCTTGCCTTT CAAACTGGAA |
| 1681 | ACCCAAGAGA AAACGGTGCC CTTGCTGTCA CCCTCTGTGG |
| 1721 | ACCCCTTTTC CCTAACTCAC TGCTCCCCAT GGGTCGGTGG |
| 1761 | CTGCAGACTG TCCCCAGGAG GGACTCTGGT TCCCTCTGTC |
| 1801 | CCCTTCTTTC CATGGGGAAC TCTGGCACCT TTCTTCTGAC |
| 1841 | CTCAGTCAAC TCTGAGCCTT ATTTCCCCCC AGGAAGTGGC |
| 1881 | CTAGGAGAAG CTACAGGGCC TAGGGACTTA CCCTGAGCTT |
| 1921 | GTAACTGGAA GACCCCGTCC CTATCCCCGC TCCCGCCCCC |
| 1961 | ACCCCACCCC ACCCCTGCTC TGGCCCCAGC CTCTGGAGGC |
| 2001 | CAGCCTTTTG GCGGGACTGA AGCCGGGCAT GGCCAACCTT |
| 2041 | GCCCACAAGT GTTTTTCTGG ATCTTGGCTG GAAGGCAGTC |
| 2081 | TGTCCCATCC TGCAGTGTTT GGGCCTGGCT CTTTGACTCA |
| 2121 | AAGCTAGCTA GGTGGCACTC CGTGTCGCTC CTGCACATTC |
| 2161 | TGGAAGGGGC GGGCCTCTCA CCCACCTCAT TCCTTTTCCC |
| 2201 | CCTGGCCTGA CTGGAAGCAG AAAAATGACC AAATCAGTAT |
| 2241 | TTTTTTTTTT TTCTTTAAGG AAATGTTACT GTTGAAAGGC |
| 2281 | CCTAGGCAAG CCTGCCCTGT TGGTTGTAGT CGTGAGTGGT |
| 2321 | CTTGGGGGGA GATGCTTGGC TCCTGTCCCT GCCTCCCCAG |
| 2361 | CGGGTTCCCT CCCTCCCTCC TGCCTGACCA CCCCAGCTCT |
| 2401 | GGCTCTGTGA TTGGTGCTCC ACGTCTTCCC AGACACCTCG |
| 2441 | GGGCTCCTGG GCGGGAGAAA GCCGGATGTG CCCCTCCCTG |
| 2481 | GGAGCCCTGG AGTAAACCTC AGGGGGCCCT TTCCCAATCA |
| 2521 | CCCCCTTCCA CCGACCCCTC AACACCATGC ATCTCACTCT |
| 2561 | GGGTGTCTCG CTCCTTTATT TTTTTGTAAC TGTCATTTCT |
| 2601 | ATAACTCTGA AGACCCATGA TAGTAAGCTT TGAACTGGAA |
| 2641 | AATAAAGTAA AATCAAGTCT GCGGCCC |

In some embodiments, the fascin polypeptide is a truncated polypeptide that includes the actin binding site and/or the binding site for migrastatin analogs. As illustrated in more detail below, fascin binds migrastatin analogs and the fascin binding site for such migrastatin analogs includes fascin amino acid residues His392, Glu391, Ala488, Lys471, His474 and Asp473. Moreover, fascin also has two actin binding sites. One of these two sites is located in the same cleft as the binding site for migrastatin analogs. The second actin binding includes amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

One example of a truncated fascin polypeptide that can be used in the invention is any fascin peptide having fascin amino acids 259 through 493, which can fold properly to generate the actin and/or migrastatin binding sites. Thus, for example, a fascin peptide having amino acids 259 through 493 of SEQ ID NO:1 has the following sequence (SEQ ID NO:9).

| | |
|---|---|
| 259 | SC AQVVLQAANE RNVSTRQGMD |
| 281 | LSANQDEETD QETFQLEIDR DTKKCAFRTH TGKYWTLTAT |
| 321 | GGVQSTASSK NASCYFDIEW RDRRITLRAS NGKFVTSKKN |
| 361 | GQLAASVETA GDSELFLMKL INRPIIVFRG EHGFIGCRKV |
| 401 | TGTLDANRSS YDVFQLEFND GAYNIKDSTG KYWTVGSDSA |
| 441 | VTSSGDTPVD FFFEFCDYNK VAIKVGGRYL KGDHAGVLKA |
| 481 | SAETVDPASL WEY |

Another example, of a fascin peptide having amino acids 259 through 493 of SEQ ID NO:3 has the following sequence (SEQ ID NO:10).

| | |
|---|---|
| 259 | PQ VVLVAANHRY VSVRQGVNVS |
| 281 | ANQDDELDHE TFLMQIDQET KKCTFYSSTG GYWTLVTHGG |
| 321 | IHATATQVSA NTMFEMEWRG RRVALKASNG RYVCMKKNGQ |
| 361 | LAAISDFVGP PPRPAWTGKV AGGAAQQTLS PPGKDEEFTL |
| 401 | KLINRPILVL RGLDGFVCHH RGSNQLDTNR SVYDVFHLSF |
| 441 | SDGAYRIRGR DGGFWYTGSH GSVCSDGERA EDFVFEFRER |
| 481 | GRLAIRARSG KYLRGGASGL LRADADAPAG TALWEY |

Another example, of a fascin peptide having amino acids 259 through 493 of SEQ ID NO:5 has the following sequence (SEQ ID NO:11).

| | |
|---|---|
| 259 | LQ HCPTWVSLRS KTGRFISVIY |
| 281 | DGEVRAASER LNRMSLFQFE CDSESPTVQL RSANGYYLSQ |
| 321 | RRHRAVMADG HPLESDTFFR MHWNCGRIIL QSCRGRFLGI |
| 361 | APNSLLMANV ILPGPNEEFG ILFANRSFLV LRGRYGYVGS |
| 401 | SSGHDLIQCN QDQPDRIHLL PCRPGIYHFQ AQGGSFWSIT |
| 441 | SFGTFRPWGK FALNFCIELQ GSNLLTVLAP NGFYMRADQS |
| 481 | GTLLADSEDI TRECIWEF |

Another example, of a fascin peptide having amino acids 259 through 493 of SEQ ID NO:7 has the following sequence (SEQ ID NO:12).

| | |
|---|---|
| 259 | SC AQVVLQAANE RNVSTRQGMD |
| 281 | LSANQDEETD QETFQLEIDR DTRKCAFRTH TGKYWTLTAT |
| 321 | GGVQSTASTK NASCYFDIEW CDRRITLRAS NGKFVTAKKN |
| 361 | GQLAASVETA GDSELFLMKL INRPIIVFRG EHGFIGCRKV |
| 401 | TGTLDANRSS YDVFQLEFND GAYNIKDSTG KYWTVGSDSS |
| 441 | VTSSSDTPVD FFLEFCDYNK VALKVGGRYL KGDHAGVLKA |
| 481 | CAETIDPASL WEY |

Such fascin peptides are useful as therapeutic agents and as antigens for generating anti-fascin antibodies. As illustrated and described herein, metastatic cancer is associated with increased expression and/or activity of fascin. Thus, agents that compete with or inhibit fascin expression and fascin activity are useful therapeutic agents for treating cancer, particularly metastatic cancer. For example, peptides having fascin amino acids 259 through 493 can compete with fascin in vivo and can inhibit endogenous fascin performing its usual role in promoting cancer metastasis. Moreover, administration of peptides having fascin amino acids 259 through 493 can immunize the mammal against endogenously produced fascin, particularly against the actin and/or migrastatin binding sites of fascin. Antibodies generated in the immunized animals serve to prevent fascin from binding to actin. Alternatively, such antibodies can mimic the inhibitory effects of migrastatin analogs by binding to the migrastatin binding site of fascin.

### Inhibitory Nucleic Acids

Nucleic acids that can inhibit the expression and/or translation of fascin can be employed in the methods of the invention described herein. Such an inhibitory nucleic acid can bind to a fascin nucleic acid, for example, a fascin RNA with a sequence corresponding to any of SEQ ID NOs: 2, 4, 6, or 8. An inhibitory nucleic acid is a polymer of ribose nucleotides or deoxyribose nucleotides having more than three nucleotides in length. An inhibitory nucleic acid may include naturally-occurring nucleotides; synthetic, modified, or pseudo-nucleotides such as phosphorothiolates; as well as nucleotides having a detectable label such as ³²P, biotin, fluorescent dye or digoxigenin. An inhibitory nucleic acid that can reduce the expression and/or activity of a fascin nucleic acid may be completely complementary to the fascin nucleic acid. Alternatively, some variability between the sequences may be permitted.

An inhibitory nucleic acid of the invention can hybridize to a fascin nucleic acid (e.g., any of SEQ ID NOs: 2, 4, 6, or 8) under intracellular conditions or under stringent hybridization conditions. The inhibitory nucleic acids of the invention are sufficiently complementary to endogenous fascin nucleic acids to inhibit expression of a fascin nucleic acid under either or both conditions. Intracellular conditions refer to conditions such as temperature, pH and salt concentrations typically found inside a cell, e.g. a mammalian cell. One example of such a mammalian cell is a cancer cell (e.g., a metastatic cell), or any cell where fascin is or may be expressed.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory nucleic acids that comprise, for example, 2, 3, 4, or 5 or more stretches of contiguous nucleotides that are precisely complementary to a fascin coding sequence, each separated by a stretch of contiguous nucleotides that are not complementary to adjacent coding sequences, may inhibit the function of a fascin nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a ribozyme or an antisense nucleic acid molecule.

An antisense nucleic acid molecule may be single or double stranded (e.g. a small interfering RNA (siRNA)), and may function in an enzyme-dependent manner or by steric blocking. Antisense molecules that function in an enzyme-dependent manner include forms dependent on RNase H activity to degrade target mRNA. These include single-stranded DNA, RNA and phosphorothioate molecules, as well as the double-stranded RNAi/siRNA system that involves target mRNA recognition through sense-antisense strand pairing followed by degradation of the target mRNA or by the RNA-induced silencing complex. Steric blocking antisense, which are RNase-H independent, interferes with gene expression or other mRNA-dependent cellular processes by binding to a target mRNA and getting in the way of other processes. Steric blocking antisense includes 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce fascin translation such that the level of fascin polypeptide is reduced. siRNAs mediate post-transcriptional gene silencing in a sequence-specific manner. See, for example, http://www.ambion.com/techlib/hottopics/rnai/rnai_may2002_print.html (last retrieved May 10, 2006). Once incorporated into an RNA-induced silencing complex, siRNAs mediate cleavage of the homologous endogenous mRNA transcript by guiding the complex to the homologous mRNA transcript, which is then cleaved by the complex. The siRNA may be homologous to any region of the fascin transcript. The region of homology may be 30 nucleotides or less in length, preferably less than 25 nucleotides, more preferably about 21 to 23 nucleotides, most preferably about 19 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site that begins with AA, has 3′ UU overhangs for both the sense and antisense siRNA strands, and has an approximate 50% G/C content. siRNAs may be chemically synthesized, created by in vitro transcription, or expressed from an siRNA expression vector or a PCR expression cassette. See, e.g., http://www.ambion.com/techlib/tb/tb_{—}506html (last retrieved May 10, 2006). Chemically synthesized siRNA relies on the same solid-phase support chemistry used to generate DNA primers for PCR. Expression or viral vectors and their RNA polymerase III (Pol III) promoters drive the expression of either siRNA transcripts, as separate sense and antisense strands that anneal in the cell, or a single short hairpin RNA transcript (Paddison, P. J. et al. (2002) Genes Dev. 16, 948-958; Sui, G. et al. (2002) Proc. Natl. Acad. Sci. U.S.A. 99, 6047-6052; Paul, C. P. et al. (2002) Nat. Biotechnol. 20, 505-508; Miyagishi M, et al. (2002) Nat. Biotechno1.20, 497-500). Human and mouse U6 and the human H1 are the most commonly used RNA polymerase III promoters. The polymerase III enzyme initiates and terminates RNA transcripts at well-defined positions (Goomer R S, et al. (1992) Nucleic Acids Res. September 25; 20(18):4903-12) making its promoters well suited for the synthesis of siRNA or shRNA.

The short length of these Pol III promoters (less than 300 bp) facilitates the generation of expression cassettes using PCR methods to amplify a linear fragment of double-stranded DNA containing the necessary promoters and the siRNA or shRNA sequence (Catanotto, D. et al. (2002) RNA 8, 1454-1460). Either the cassette itself or the purified in vitro transcript of the cassette serves as the source of nucleic acid for RNAi.

Finally, treatment of dsRNA in vitro with purified mammalian Dicer or the E. coli enzyme RNase III digests the nucleic acid into a population of siRNA duplexes. Generation of the dsRNA involves the in vitro transcription of both strands of either a gene-specific fragment or a full-length cDNA of the gene of interest cloned into an appropriate vector.

When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

Table 1 illustrates siRNA target sequences of human fascin useful in the invention described herein.

TABLE 1

| siRNA Target Sequence | SEQ ID NO: |
|---|---|
| CCAGCAGCCTGAAGAAGAA | 13 |
| GGCAAGTACTGGACGCTGA | 14 |
| CAAAGACTCCACAGGCAAA | 15 |
| ATAACAAGGTGGCCATCAA | 16 |
| CTGAAGGCCTCGGCGGAAA | 17 |
| TCAAAGACTCCACAGGCAA | 18 |
| AGACCGACCAGGAGACCTT | 19 |
| CTGAAGAAGAAGCAGATCT | 20 |
| CCTTCCAGGACCAGCGCTA | 21 |
| CCAAGGTGGGCAAGGACGA | 22 |
| ACTATAACAAGGTGGCCAT | 23 |
| GCGTTCGGGTTCAAGGTGA | 24 |
| GCCAGCAGCCTGAAGAAGA | 25 |
| AAGAAGAAGCAGATCTGGA | 26 |
| GTCAACATCTACAGCGTCA | 27 |
| GTATGGACCTGTCTGCCAA | 28 |
| GCGCCTACAACATCAAAGA | 29 |
| GCGACACTCCTGTGGACTT | 30 |
| CCGCCAGCAGCCTGAAGAA | 31 |
| AGAAGTGGAGCGTGCACAT | 32 |
| TGGGCAAGGACGAGCTCTT | 33 |
| GCCTGAAGAAGAAGCAGAT | 34 |
| CCAATCAGGACGAGGAGAC | 35 |
| GAGGAGACCGACCAGGAGA | 36 |
| AGATCGACCGCGACACCAA | 37 |
| GAGCATGGCTTCATCGGCT | 38 |
| TGTCCACGCGCCAGGGTAT | 39 |
| GCTGCTACTTTGACATCGA | 40 |
| GGCAAATACTGGACGGTGG | 41 |
| AGCCTGAAGAAGAAGCAGA | 42 |
| GGTATGGACCTGTCTGCCA | 43 |
| CTGCCAATCAGGACGAGGA | 44 |
| TTTGTGACCTCCAAGAAGA | 45 |
| ACGCCAACCGCTCCAGCTA | 46 |
| CCTACAACATCAAAGACTC | 47 |
| ATCAAAGACTCCACAGGCA | 48 |
| AGTTCTGCGACTATAACAA | 49 |
| GACAAGGACGGCAACGTGA | 50 |
| AGGTCAACATCTACAGCGT | 51 |
| ACGAGGAGACCGACCAGGA | 52 |
| GCAAGTTTGTGACCTCCAA | 53 |
| TGAAGAAGAAGCAGATCTG | 54 |
| TCGAGTTCTGCGACTATAA | 55 |
| AGATCTGGACGCTGGAGCA | 56 |
| GCCTACAACATCAAAGACT | 57 |
| CTTCGAGTTCTGCGACTAT | 58 |
| ACCCTCAGGTCAACATCTA | 59 |
| TCAACTGCGGCAACAAGTA | 60 |
| CTGGAGATCGACCGCGACA | 61 |
| CCTCCAAGAAGAATGGGCA | 62 |

An antisense inhibitory nucleic acid may also be used to specifically reduce fascin expression, for example, by inhibiting transcription and/or translation. An antisense inhibitory nucleic acid is complementary to a sense nucleic acid encoding fascin. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding fascin. The non-coding region includes the 5′ and 3′ regions that flank the coding region, for example, the 5′ and 3′ untranslated sequences. An antisense inhibitory nucleic acid is generally at least six nucleotides in length, but may be about 8, 12, 15, 20, 25, 30, 35, 40, 45, or 50 nucleotides long. Longer inhibitory nucleic acids may also be used.

An antisense inhibitory nucleic acid may be prepared using methods known in the art, for example, by expression from an expression vector encoding the antisense inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the antisense inhibitory nucleic acid and the sense nucleic acid.

Naturally-occurring nucleotides include the ribose or deoxyribose nucleotides adenosine, guanine, cytosine, thymine and uracil.

Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, butoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

An inhibitor of the invention can also be a small hairpin RNA or short hairpin RNA (shRNA) is a sequence of RNA that makes a tight hairpin turn that can be used to silence gene expression via RNA interference. The shRNA hairpin structure is cleaved by the cellular machinery into an siRNA, which is then binds to and cleaves the target mRNA. shRNA can be introduced into cells via a vector encoding the shRNA, where the shRNA coding region is operably linked to a promoter. The selected promoter permits expression of the shRNA. For example, the promoter can be a U6 promoter, which is useful for continuous expression of the shRNA. The vector can, for example, be passed on to daughter cells, allowing the gene silencing to be inherited. See, McIntyre G, Fanning G, Design and cloning strategies for constructing shRNA expression vectors, BMC BIOTECHNOL. 6:1 (2006); Paddison et al., Short hairpin RNAs (shRNAs) induce sequence-specific silencing in mammalian cells, GENES DEV. 16 (8): 948-58 (2002).

An inhibitor of the invention may also be a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a fascin mRNA transcript and thereby inhibit translation of the mRNA. See, for example, Haseloff et al., U.S. Pat. No. 5,641,673.

Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 5, 6, 7, 8, 9, 10, 12, 15, 20, or 50 contiguous nucleotides selected from a specific nucleotide sequence. Longer complementary sequences may be used to increase the affinity of the hybridization sequence for the target.

The hybridizing and cleavage regions of the ribozyme can be integrally related; thus, upon hybridizing to the target RNA through the complementary regions, the catalytic region of the ribozyme can cleave the target. Thus, an existing ribozyme may be modified to target a fascin nucleic acid of the invention by modifying the hybridization region of the ribozyme to include a sequence that is complementary to the target fascin nucleic acid. Alternatively, an mRNA encoding a fascin may be used to select a catalytic RNA having a specific ribonuclease activity from a pool of RNA molecules. See, for example, Bartel & Szostak, Science 261:1411-1418 (1993).

Thus, inhibitory nucleic acids of the invention may include modified nucleotides, as well as natural nucleotides such as combinations of ribose and deoxyribose nucleotides, and an antisense inhibitory nucleic acid of the invention may be of any length discussed above and that is complementary to fascin.

In some embodiments, expression cassettes are employed in the various embodiments described herein. Expression cassettes can be of any suitable construction, and can be included in any appropriate delivery vector. Such delivery vectors include plasmid DNA, viral DNA, and the like. The means by which the expression cassette in its delivery or expression vector is introduced into target cells or target organism can be transfection, reverse transfection, virus induced transfection, electroporation, direct introduction by biolystics (e.g., using a “gene gun;” BioRad, Inc., Emeryville, Calif.), and the like. Other methods that can be employed include methods widely known in the art as the methods of gene therapy. Once delivered into a target cell, or target organism the expression cassette may be maintained on an autonomously replicating piece of DNA (e.g., an expression vector), or may be integrated into the genome of the target cell or target organism.

Typically, to assemble the expression cassettes and vectors of the present invention a nucleic acid, preferably a DNA, encoding an siRNA is incorporated into a unique restriction endonuclease cleavage site, or a multiple cloning site, within a pre-existing “empty” expression cassette to form a complete recombinant expression cassette that is capable of directing the production of the siRNA transcripts of the present invention. Frequently such complete recombinant expression cassettes reside within, or inserted into, expression vectors designed for the expression of such siRNA transcripts. Methods for the construction of an expression vector for purposes of this invention should be apparent to skilled artisans apprised of the present invention. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

Generally, the expression cassettes inserted or assembled within the expression vectors have a promoter operably linked to a DNA encoding the siRNA that is to be employed. The promoter can be a native promoter, i.e., a promoter that is responsible for the expression of that particular gene product in cells, or it can be any other suitable promoter. Alternatively, the expression cassette can be a chimera, i.e., having a heterologous promoter that is not the native promoter responsible for the expression of the siRNA. Such heterologous promoters can even be from a different species than the target cell or organism.

The expression vector may further include an origin of DNA replication for the replication of the vectors in target cells. Preferably, the expression vectors also include a replication origin for the amplification of the vectors in, e.g., E. coli, and selection marker(s) for selecting and maintaining only those target cells harboring the expression vectors. Additionally, in some embodiments the expression vectors also contain inducible or derepressible promoters, which function to control the transcription of the siRNA transcript from the DNA that encodes it. Other regulatory sequences such as transcriptional enhancer sequences and translation regulation sequences (e.g., Shine-Dalgarno sequence) can also be operably included in the expression vectors. Transcription termination sequences, and polyadenylation signal sequences, such as those from bovine growth hormone, SV40, lacZ and AcMNPV polyhedral protein genes, may also be present.

The expression vectors of the present invention can be introduced into the target cells by any techniques known in the art, e.g., by direct DNA transformation, microinjection, electroporation, viral infection, lipofection, biolystics, and the like. The expression of the siRNA can be transient or stable, inducible or derepressible. The expression vectors can be maintained in target cells in an extrachromosomal state, i.e., as self-replicating plasmids or viruses. Alternatively, the expression vectors, or portions thereof, can be integrated into chromosomes of the target cells by conventional techniques such as site-specific recombination or selection of stable cell lines. In stable cell lines, at least the expression cassette portion of the expression vector is integrated into a chromosome of the target cells.

The vector construct can be designed to be suitable for expression in various target cells, including but not limited to bacteria, yeast cells, plant cells, nematode cells, insect cells, and mammalian and human cells. Methods for preparing expression vectors designed for expression of gene products in different target cells are well known in the art.

### Migrastatin Analogs

Migrastatin (1) is an inhibitor of cell migration. Nakae et al., J. Antibiot. 2000, 53, 1130; Nakae et al., J. Antibiot. 2000, 53, 1228; Takemoto et al., J. Antibiot. 2001, 54, 1104; Nakamura et al., J. Antibiot. 2002, 55, 442; Woo et al. J. Antibiot. 2002, 55, 141. The structure of migrastatin is provided below.

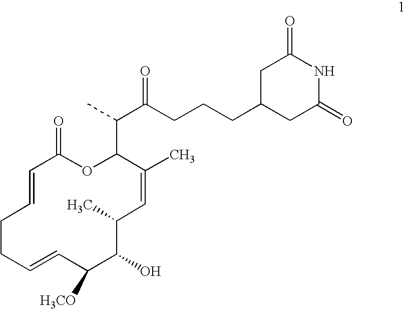

According to the invention, analogs of migration bind to fascin and inhibit the activity of fascin.

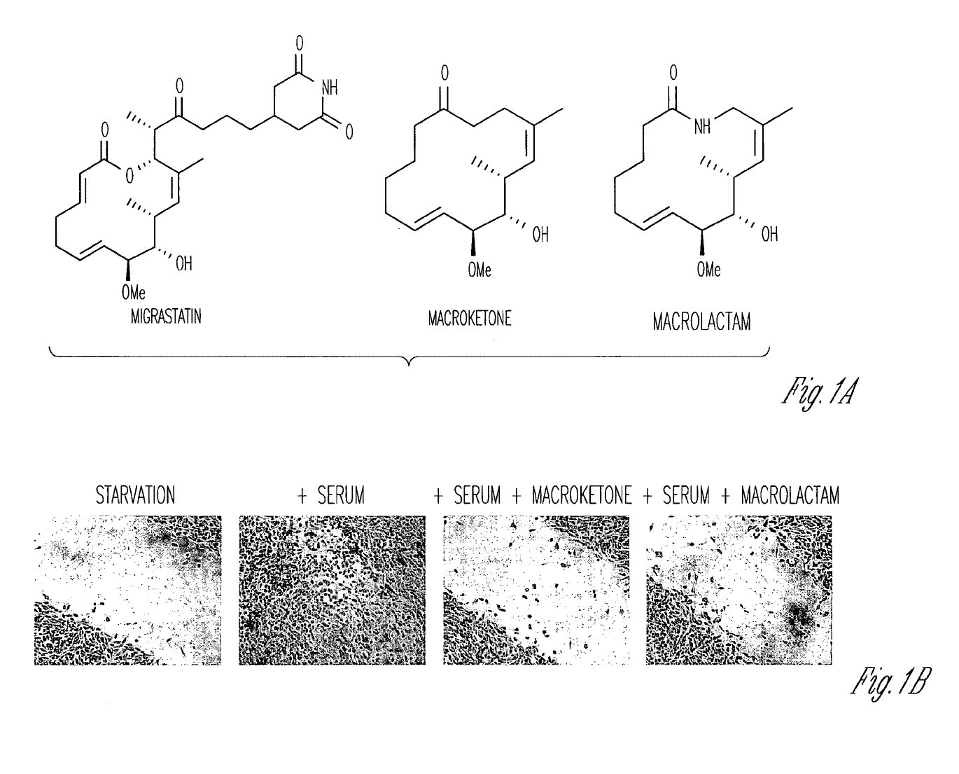

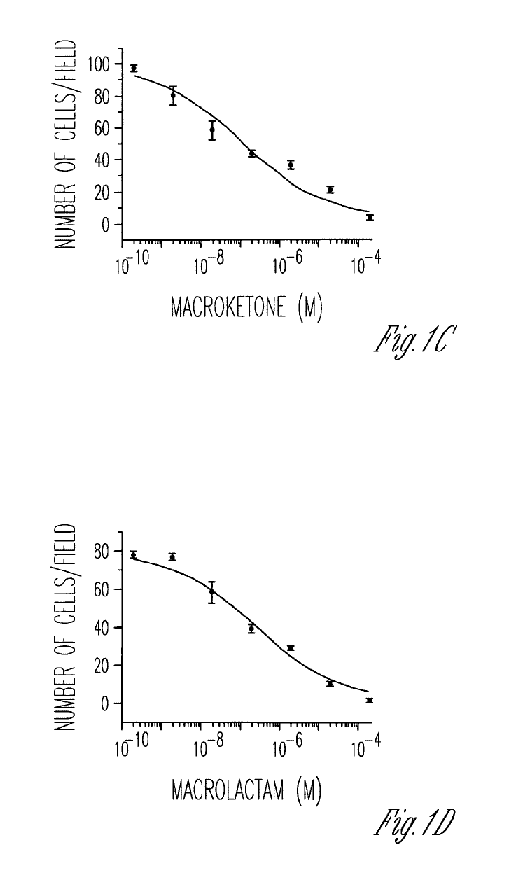

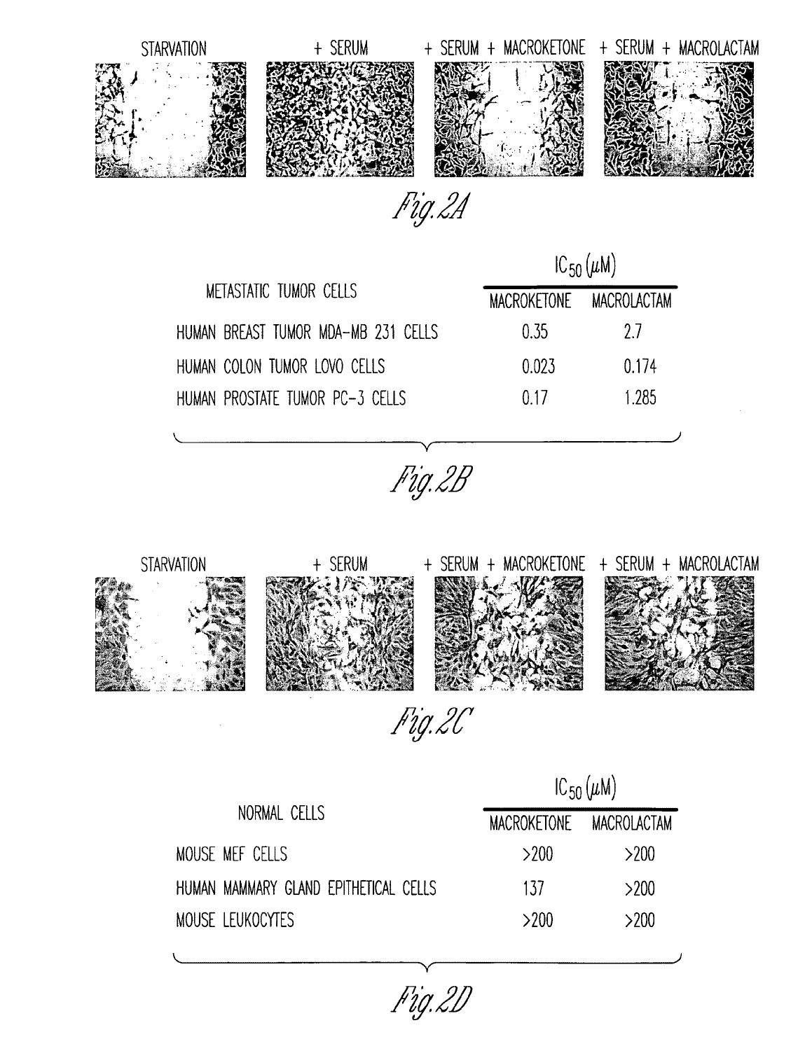

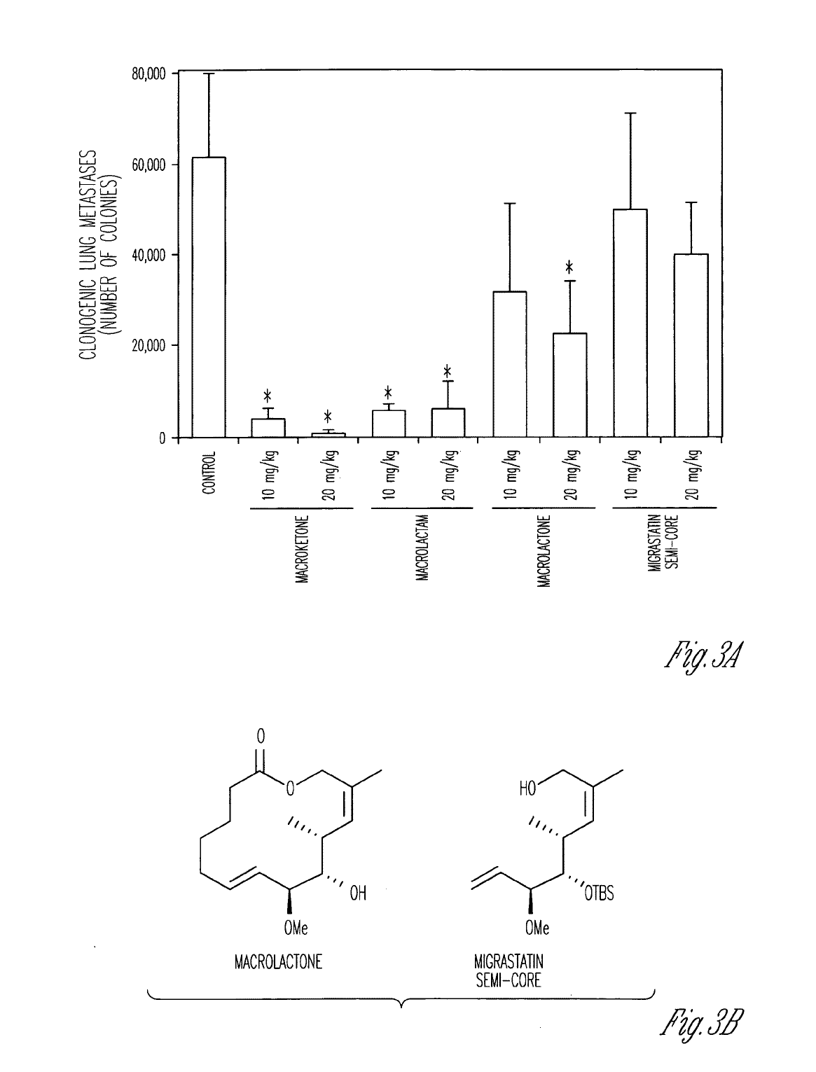

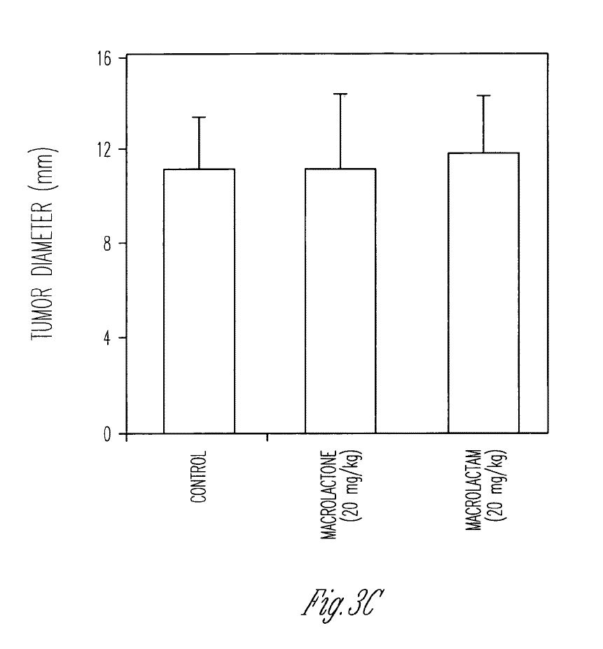

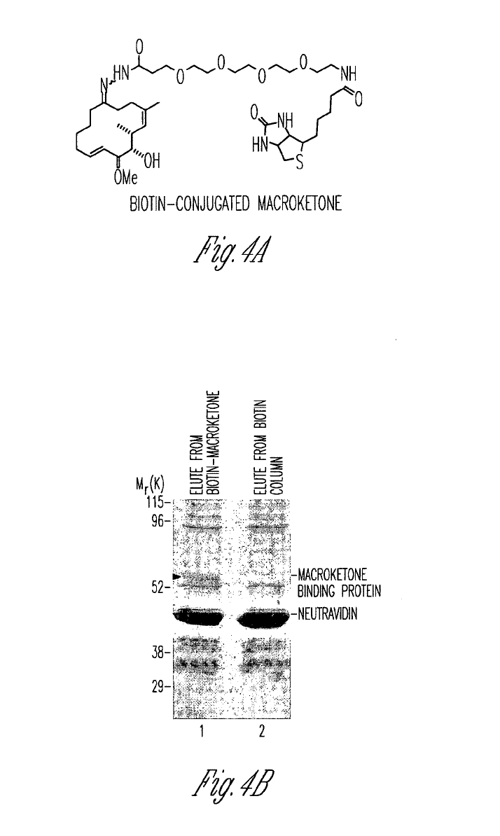

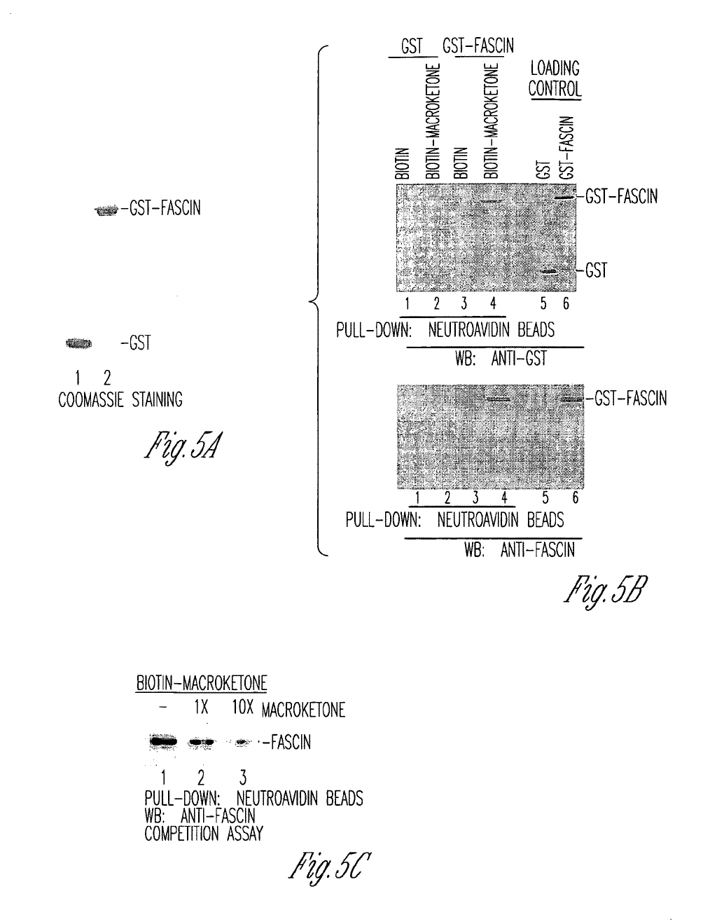

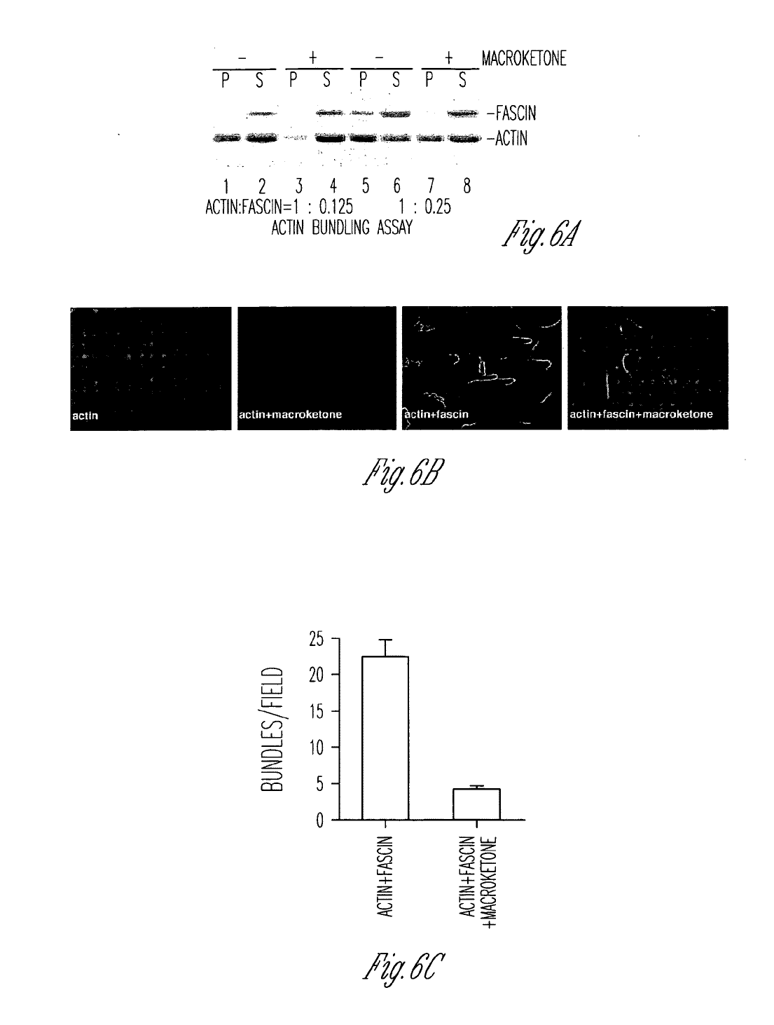

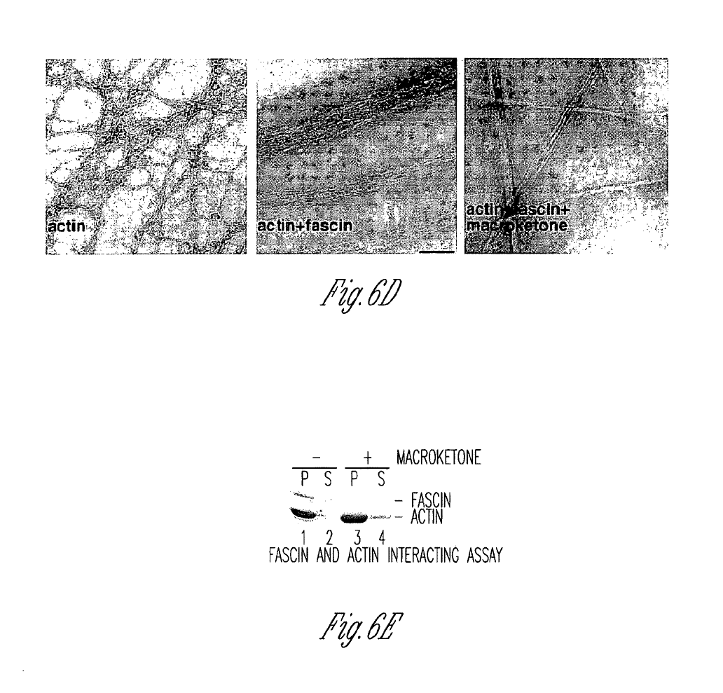

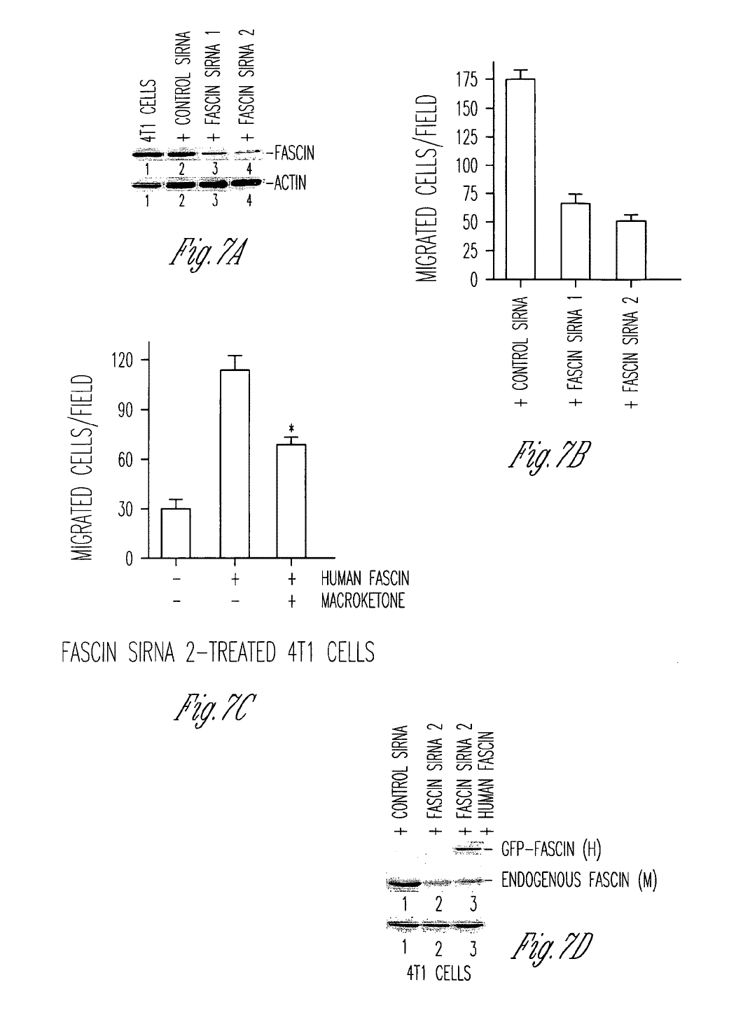

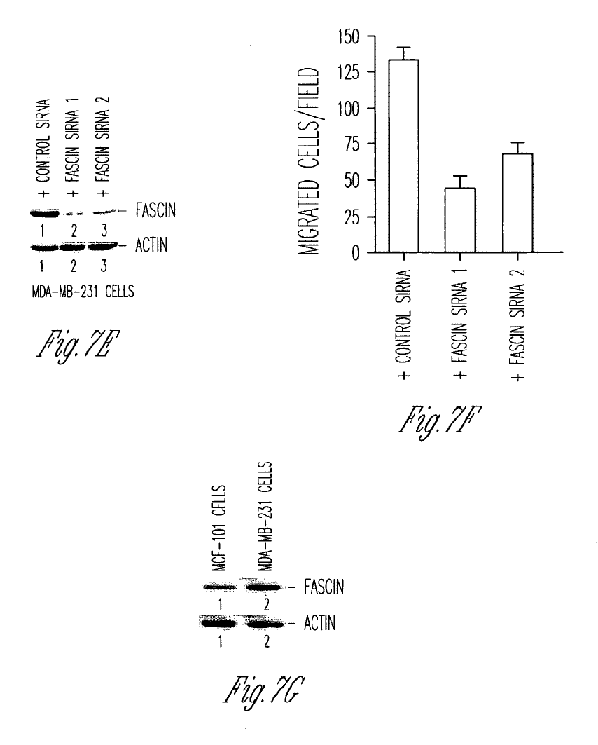

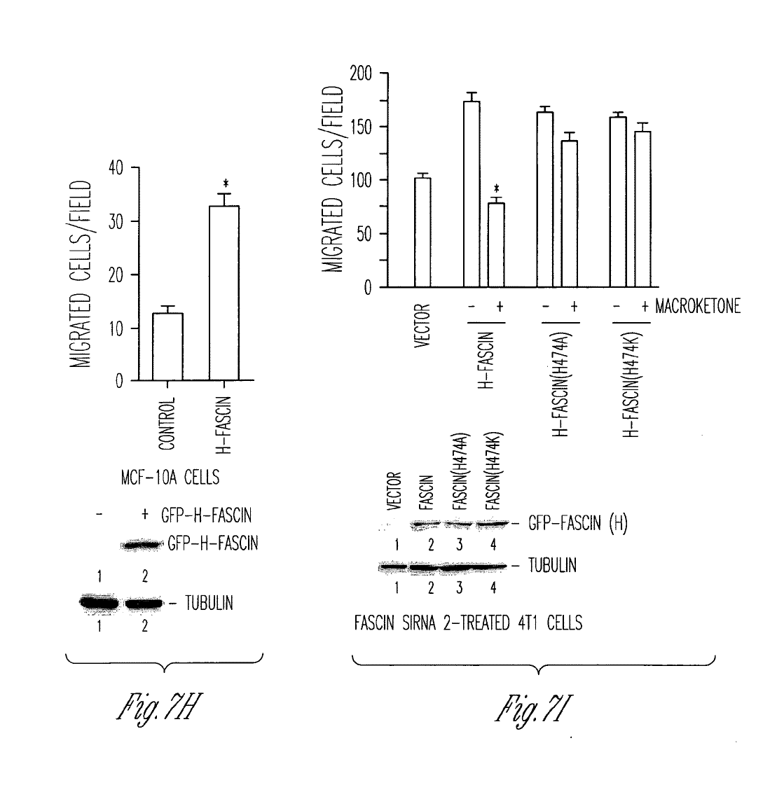

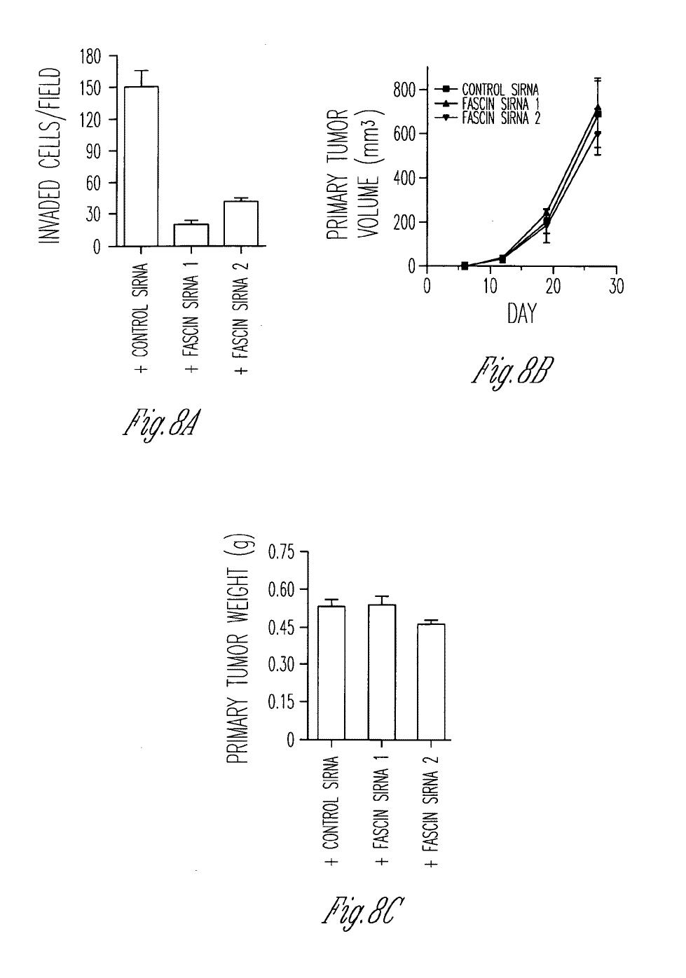

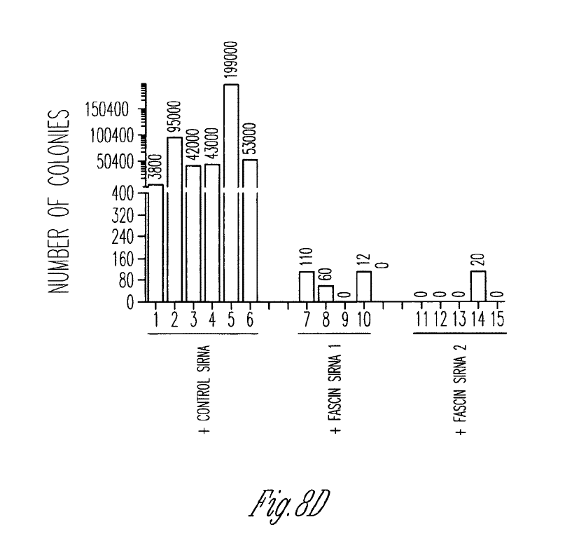

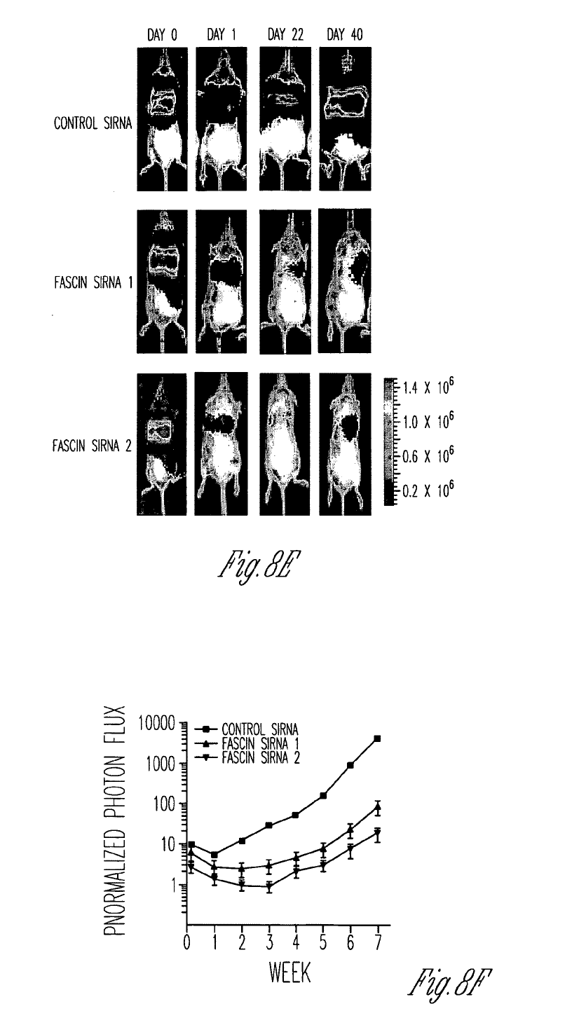

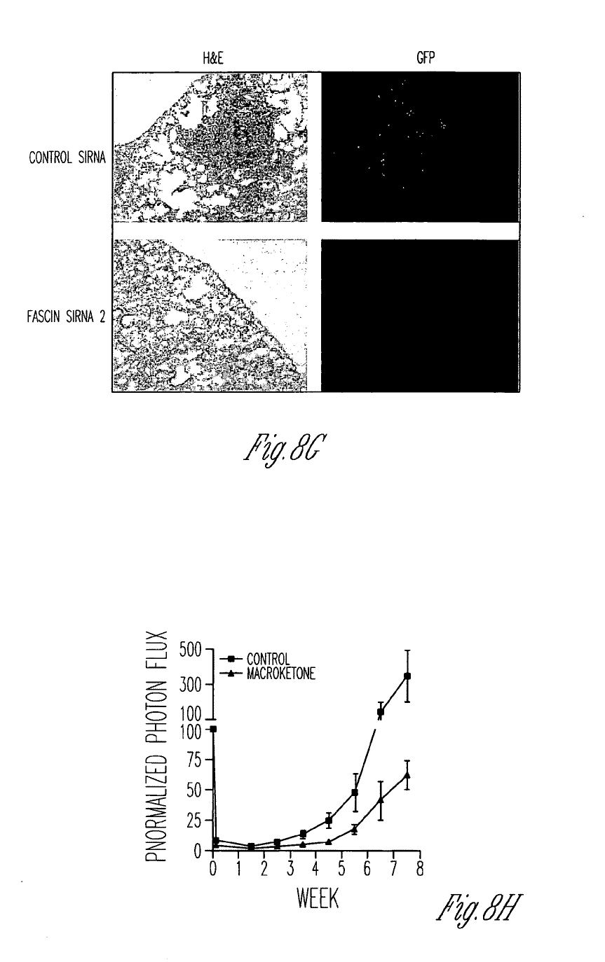

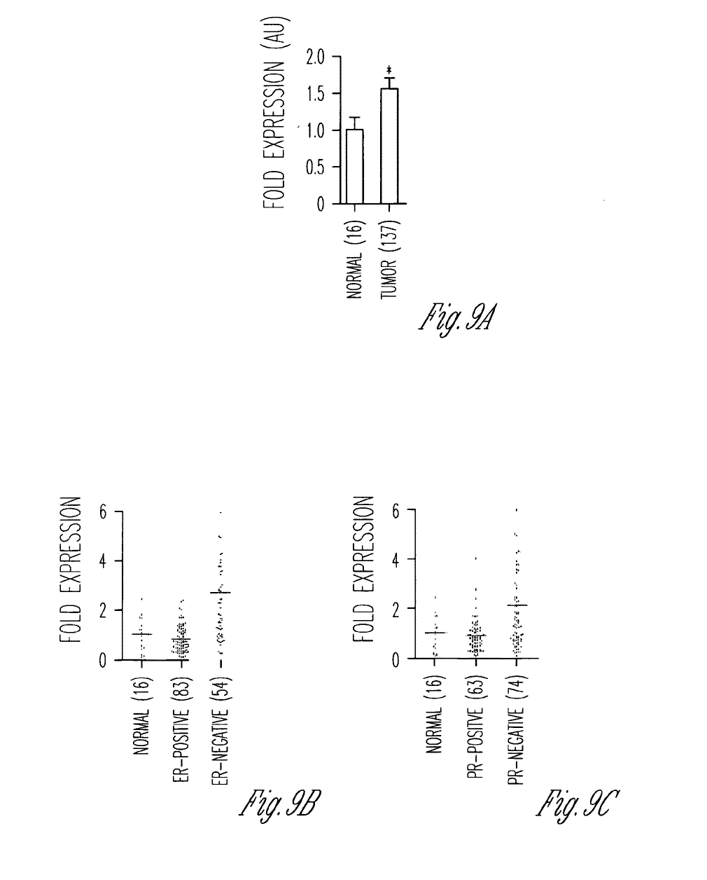

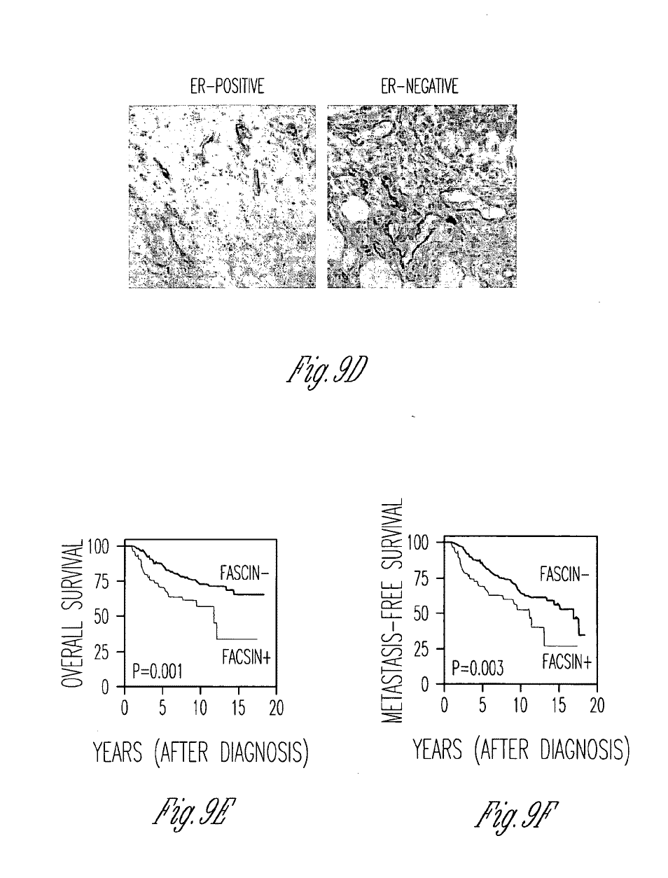

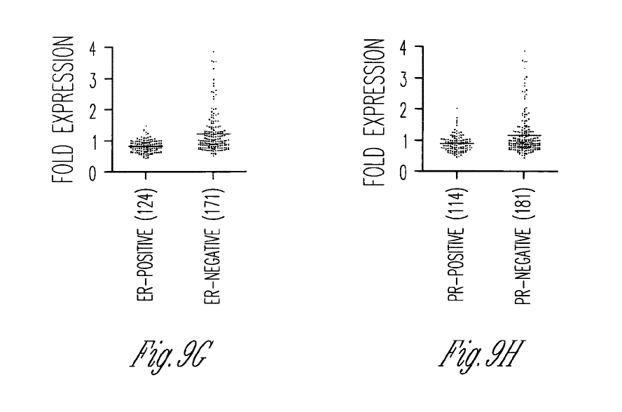

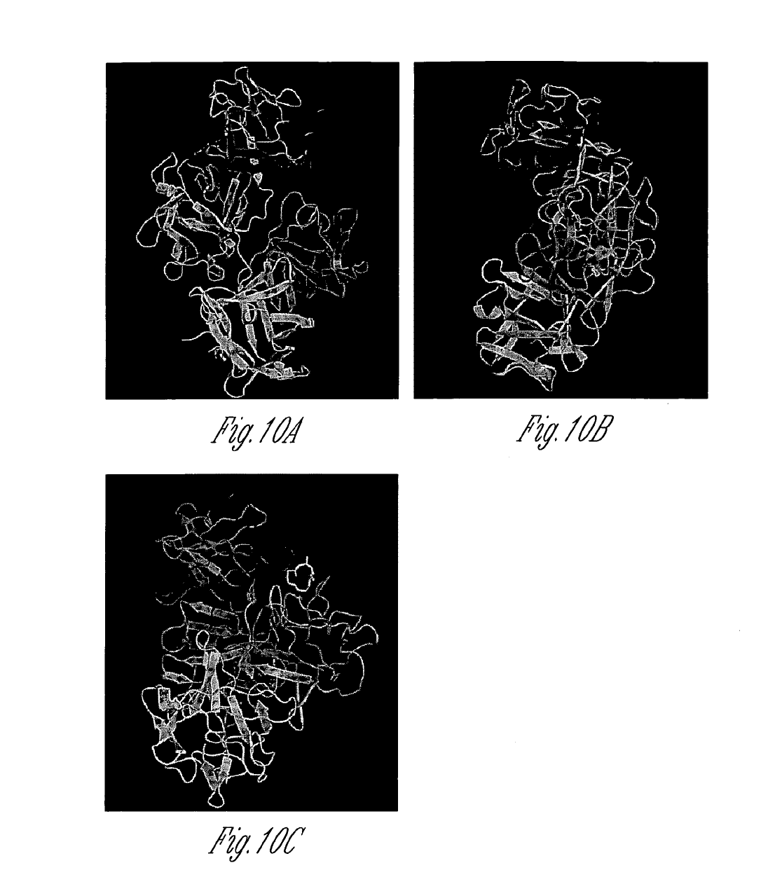

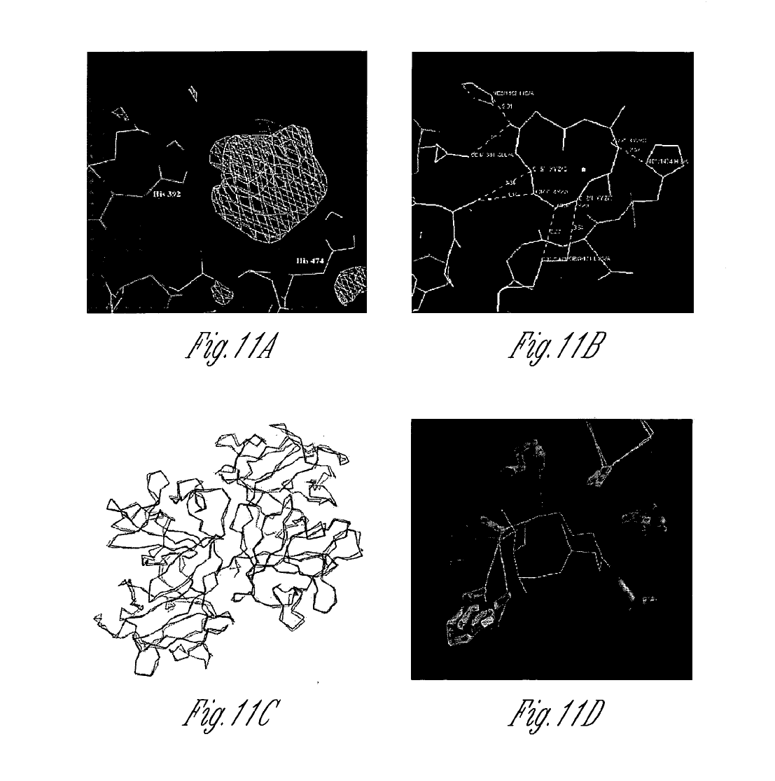

Migrastatin is a macrolide natural product first isolated from a cultured broth of Streptomyces and its structure features a 14-membered macrolactone ring (FIG. 1A) (Nakae et al. 2000, Woo et al. 2002). At high micromolar concentrations, the natural product inhibits the migration of several types of tumor cells in vitro but has no effect on the biosyntheses of DNA, RNA, and protein in these cells (Nakae et al. 2000).

Two synthetic migrastatin analogs, a core macroketone and a core macrolactam (FIG. 1A), were tested for inhibition of mouse breast tumor metastasis in a mouse model (Shan et al. 2005). These two compounds are potent inhibitors of mouse breast tumor metastasis, reducing 91-99% of tumor spreading to the lung. It has been determined that the cellular basis for this effect is the interference of the formation of lamellipodia that, in turn, inhibits migration of breast tumor cells. It has been further determined that the compounds of the invention exert this effect by interacting with and inhibiting fascin.

The following definitions are used, unless otherwise described: halo is fluoro, chloro, bromo, or iodo. Alkyl, alkoxy, alkenyl, alkynyl, etc. denote both straight and branched groups.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine the cell migration inhibitory activity of such forms using the standard tests described herein, or using other similar tests which are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy.

In some embodiments, the compounds of formula I have the following structures, or pharmaceutically acceptable salts thereof.

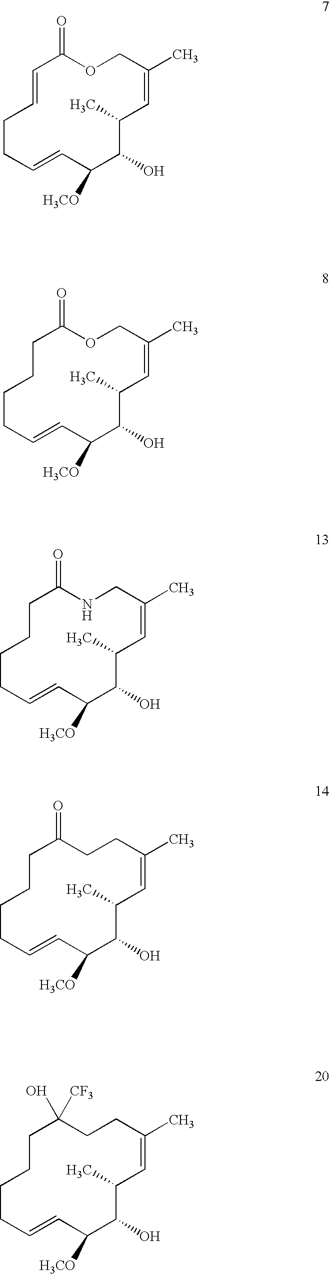

Procedures available in the art can be used for synthesizing the compounds of the invention. For example, the compounds of the invention can be made as described in Njardarson et al., J. Am. Chem. Soc. 2004, 126, 1038-1040.

Further details on synthesizing organic compounds can be found in the art, for example, in Greene, T. W.; Wutz, P. G. M. “Protecting Groups In Organic Synthesis” second edition, 1991, New York, John Wiley & sons, Inc. The Examples provided herein further illustrate synthetic procedures for the compounds of formula I.

In cases where compounds (e.g., the migrastatin analogs and inhibitory nucleic acids described herein) are sufficiently basic or acidic to form stable nontoxic acid or base salts, administration of the compounds as salts may be appropriate. Certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences, 66: 1-19 (1977), incorporated herein by reference. The salts can be prepared in situ during the final isolation and purification of the compounds of the invention, or separately by reacting a free base or free acid function with a suitable reagent. For example, a free base function can be reacted with a suitable acid.

Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and other mammals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of formula I described herein, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

### Anti-Fascin Antibodies

The invention provides antibody preparations directed against fascin, for example, antibodies capable of binding a polypeptide having SEQ ID NO:1, 3, 5, 7, 9, 10 and/or 12. In some embodiments, the antibody can bind to the actin binding sites or the migrastatin-analog binding site. For example, in some embodiments, the antibodies of the invention can bind to an epitopal site that includes any of fascin amino acid residues His392, Glu391, Ala488, Lys471, His474 and Asp473, which form key portions of the migrastatin analog binding site. In other embodiments, the antibodies of the invention can bind to an epitopal site that includes any of fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, which form key parts of one of the fascin actin binding sites.

Such antibodies are desirable to block the activity of fascin, which, as illustrated herein, is associated with metastatic cancer and tumors. Thus, antibody preparations of the invention can serve as inhibitors of fascin activity and therefore act as therapeutic agents.

Methods are provided to prepare and screen for antibodies that preferentially recognize fascin, the fascin-actin binding sites and/or the fascin-migrastatin analog binding site. A peptide sequence that includes fascin amino acid residues His392, Glu391, Ala488, Lys471, His474 and Asp473 (the migrastatin analog binding site) and/or fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250 (one of the actin binding sites) is used as antigen to raise polyclonal or monoclonal antibodies. Fascin peptides that are used to generate antibodies of the invention include peptides with the above-identified epitopal sites. For example, such fascin peptides include any peptide with a sequence that includes amino acids 259 through 493 of SEQ ID NO:1, 3, 5, 7, 9, 10 and/or 12.

The resultant antibodies are selected for binding to fascin or a selected peptide sequence (e.g., the antigenic peptide used to generate the antibodies). The antibodies can then be screened for inhibition of fascin. Inhibitory antibodies are selected by screening the antibodies for inhibition as described herein, for example, as described below and in the Examples.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three complementarity-determining regions (CDRs), which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody” also refers to a portion of a full-length antibody, generally the antigen binding or variable region. Examples of antibody fragments that can serve as antibodies of the invention include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc'). Additional fragments that are included in the invention are diabodies, linear antibodies, single-chain antibody molecules, and multispecific antibodies formed from antibody fragments. In some embodiments, the antibodies are Fv, F(ab) and F(ab′)₂ fragments.

Therefore, the antibodies contemplated by the invention therefore do not have to be full-length antibodies, so long as they bind fascin with specificity. Moreover, the antibodies of the invention can include polypeptides having fascin binding domains, for example, fascin-binding complementarity-determining regions (CDRs). Such CDRs can be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody of the invention has any upper size limit so long as it binds with specificity to fascin, e.g. a polypeptide having SEQ ID NO:1, 3, 5, 7, 9, 10 and/or 12.

Antibody fragments retaining an ability to selectively bind with its antigen. Some types of antibody fragments are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv including only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further includes a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

Methods for preparing polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Methods for preparing monoclonal antibodies are likewise available to one of skill in the art. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of monoclonal antibodies are also available to those skilled in the art. For example, monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991). Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates that the antibody preparation is a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be non-covalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For example, humanized antibodies can be made from a human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

The invention also provides methods of mutating antibodies to optimize their affinity, selectivity, binding strength or other desirable property. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody. One method of mutating antibodies involves affinity maturation using phage display.

The invention is therefore directed to a method for selecting antibodies and/or antibody fragments or antibody polypeptides with desirable properties. Such desirable properties can include increased binding affinity or selectivity for fascin and/or fascin epitopes (e.g., the fascin actin or migrastatin binding sites of the invention).

The antibodies and antibody fragments of the invention are isolated antibodies and antibody fragments. An isolated antibody is one that has been identified and separated and/or recovered from a component of the environment in which it was produced. Contaminant components of its production environment are materials that would interfere with diagnostic or therapeutic uses for the antibody, and may include antigenic proteins, enzymes, hormones, and other proteinaceous or nonproteinaceous solutes. The term “isolated antibody” also includes antibodies within recombinant cells because at least one component of the antibody's natural environment will not be present. In some embodiments, however, an isolated antibody will be at least partially purified, for example, by employing at least one purification step.

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In some embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequentator; or 3) to homogeneity by SDS-PAGE under reducing or non-reducing conditions using Coomasie blue or, preferably, silver stain.

### Fascin Structure

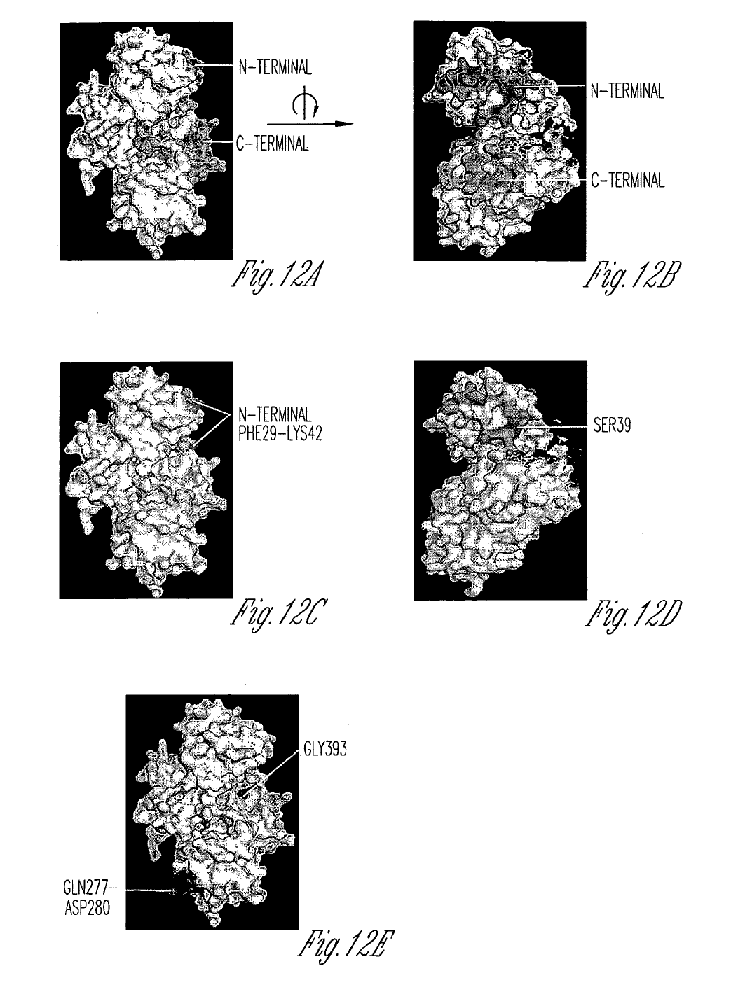

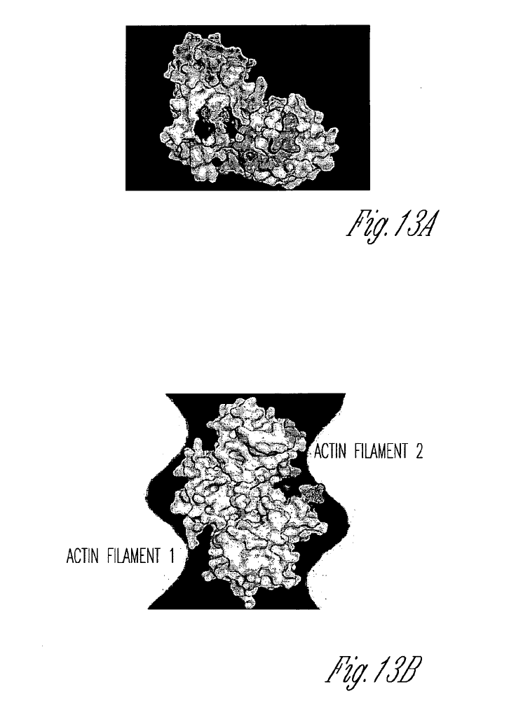

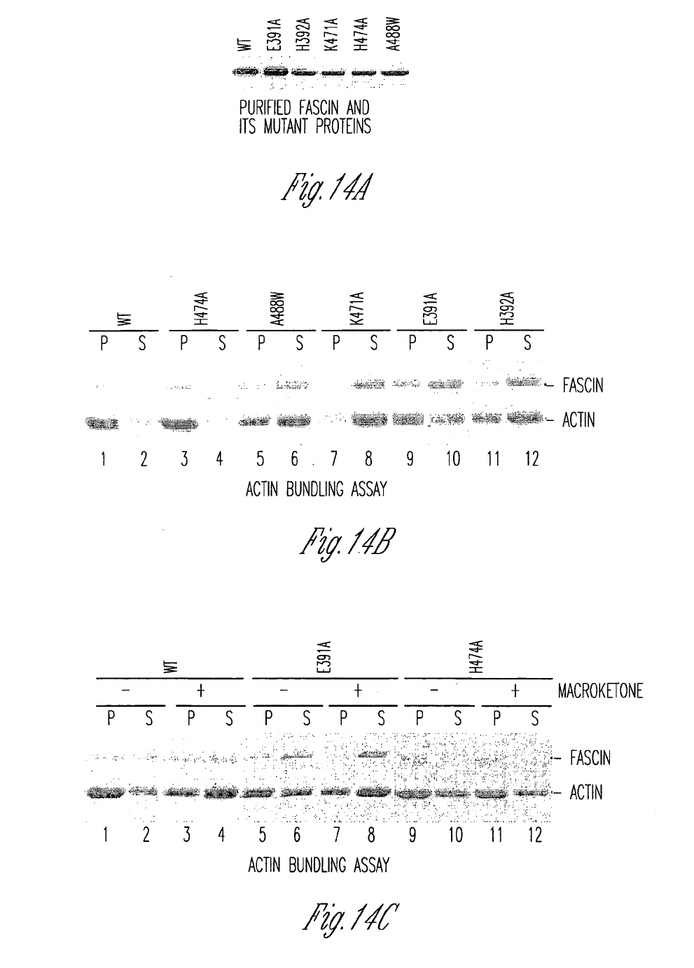

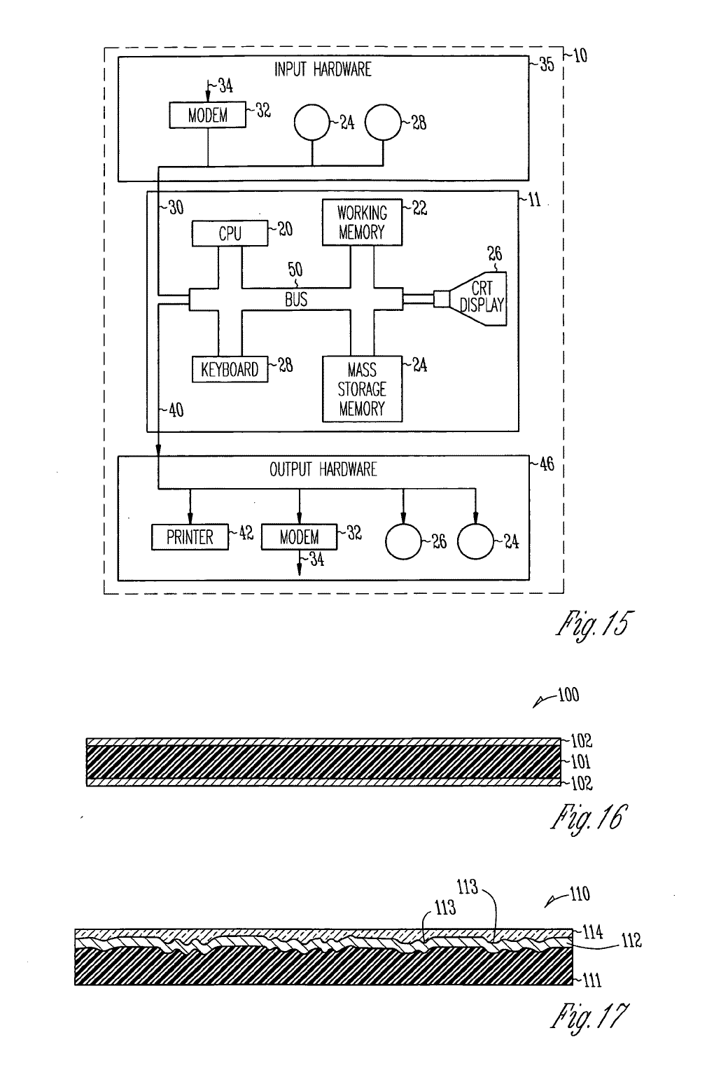

The invention further relates to the three dimensional structure of fascin. Table 2 provides the three-dimensional coordinates for the atoms in fascin. As described in more detail in Example 9, fascin has two actin binding sites. When fascin binds to actin it facilitates formation of actin bundles. For example, addition of fascin induced the formation of F-actin bundles (FIG. 6B).

One of the primary actin binding sites of fascin is the binding site for migrastatin analogs. The second actin binding site includes fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp 168, Pro159, Arg151, Lys150, Arg149, Arg197, Arg201, Glu207, Glu227, Ser237, Pro236, Lys241, Lys247, and Lys250.

Migrastatin analogs can bind to at least one of the actin binding sites and such binding inhibits actin bundling. For example, the migrastatin analog, macroketone, binds at the surface of trefoil 4, on the side facing the cleft between trefoil 4 and trefoil 1 (FIG. 10C). Macroketone is held in place by interacting with the side chains of His392, Glu391, Ala488, Lys471, and His474 as well as the alpha carbon of Asp473 (FIG. 11A-D). The six residues form a U-shape curvature, holding macroketone like holding a ring with thumb and index finger (FIGS. 11A and 11B). On the top of the two “fingers” are the two histidines, His392 and His474, which have major contributions to the fascin-macroketone interaction. The NE2 nitrogen of His392 is 3.01 Å away from the ketone oxygen of macroketone molecule, while the ND1 nitrogen of His474 is 2.57 Å away from the hydroxyl oxygen. His392 and His474 contribute to the binding of macroketone by forming hydrogen bonds with macroketone (FIG. 11B). The interaction between fascin and macroketone is further stabilized by the van der Waals force between the macrolide ring carbon and residue Glu391, Ala488, Lys471 and Asp473 (FIG. 11B).

While addition of fascin induced the formation of F-actin bundles (FIG. 6B), in the presence of macroketone, formation of F-actin bundles was largely (>80%) inhibited (FIGS. 6B and 6C).

As described herein, fascin amino acid residues His392, Glu391, Ala488, Lys471, His474 and Asp473 form portions of the migrastatin analog binding site.

Thus, as described herein, fascin has two binding sites. Actin can interact with both sites. However, the migrastatin analogs apparently interact with only one site. The migrastatin analog binding site is a U-shaped cleft or pocket with dimensions of about eight (8) by ten (10) by ten (10) angstroms (i.e., 8 Å×10 Å×10 Å). The other binding site for actin on fascin is also U-shaped, but it runs along the surface of fascin and is not an indented pocket.

Methods of Detecting and Isolating Agents that can Modulate Fascin

The invention further provides screening methods and assays that are useful for generating or identifying therapeutic agents for inhibiting fascin and the diseases associated with fascin activity.

One skilled in the art may use one of several methods to screen test agents for their ability to associate, bind and/or modulate the activity of fascin. For example, one of skill in the art may use the fascin structure described herein to identify the type, shape and structure of molecules that can interact with fascin actin and migrastatin analog binding sites. One of skill in the art may also screen test agents by observing whether a test agent binds to fascin and/or inhibits cell migration. These methods are described in more detail below.

Binding sites, also referred to as binding pockets in the present invention, are of significant utility in fields such as drug discovery. Such binding pockets or sites are the locus of fascin's actin bundling activity. Moreover, identification of the location and composition of the actin and migrastatin analog binding sites facilitates discovery of small molecules, drugs and or factors that interact with, bind and/or modulate fascin activity. An understanding of the size, structure and composition of fascin-actin and fascin-migrastatin analog binding sites also facilitates the design of drugs having more favorable associations with these binding sites, and thus, provides drugs and therapeutic agents with improved biological effects. For example, the fascin three dimensional structure and the physical and chemical properties of the fascin binding sites facilitates design of inhibitors that interact with, bind or block those binding sites.

Test agents that exhibit an appropriate size, atomic structure and chemical make-up may be tested further in actual binding assays, cell migration assays and the like to ascertain whether those test agents are viable candidates for development as therapeutic agents for inhibiting fascin in vivo. This screening process may begin by visual inspection of, for example, one of the actin or migrastatin analog binding sites on the computer screen using the fascin three dimensional atomic coordinates in Table 2 or other coordinates which define a similar shape generated from the machine-readable storage medium. Selected fragments or chemical moieties may then be positioned in a variety of orientations, or docked, within that binding site. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical moieties. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif. 4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable chemical entities or moieties have been selected, they can be assembled into a single test agent (e.g., a compound or complex). Assembly may be preceded by visual inspection of the relationship of the fragments to each other on the three-dimensional image displayed on a computer screen in relation to the structure coordinates of fascin. This would be followed by manual model building using software such as Quanta or Sybyl [Tripos Associates, St. Louis, Mo.].

Useful programs to aid one of skill in the art in selecting and joining the individual chemical moieties or fragments include: 1. CAVEAT (P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif. 2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992). 3 HOOK (M. B. Eisen et al, “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Instead of proceeding to build an modulator or inhibitor of fascin in a step-wise fashion by defining one moiety or chemical fragment at a time as described above, test agents that can bind fascin can be designed as a whole or “de novo” using either an empty binding site or optionally including some portion(s) of a known inhibitor(s). There are many de novo ligand design methods including: 1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif. 2. LEGEND (Y. Nishibata et al., Tetrahedron, 47, p. 8985 (1991)). LEGEND is available from Molecular Simulations Incorporated, San Diego, Calif. 3. LeapFrog (available from Tripos Associates, St. Louis, Mo.). 4. SPROUT (V. Gillet et al, “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., N. C Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp 337-380 (1994); see also, W. C. Guida, “Software For Structure-Based Drug Design”, Curr. Opin. Struct. Biology, 4, pp. 777-781 (1994)].

Once a test agent has been designed or selected by the above methods, the efficiency with which that test agent binds to a fascin binding site can be tested and optimized by computational evaluation. For example, an effective fascin binding site inhibitor must preferably demonstrate a relatively small difference in energy between its bound and free states (i.e., a small deformation energy of binding). Thus, the most efficient fascin binding site inhibitors should preferably be designed with a deformation energy of binding of not greater than about 10 kcal/mole, more preferably, not greater than 7 kcal/mole. Fascin binding site inhibitors may interact with the binding site in more than one conformation that is similar in overall binding energy. In those cases, the deformation energy of binding is taken to be the difference between the energy of the free entity and the average energy of the conformations observed when the inhibitor binds to the protein.

A test agent designed or selected as binding to a fascin binding site may be further computationally optimized so that in its bound state it would preferably lack repulsive electrostatic interaction with the target binding site and with the surrounding water molecules. Such non-complementary electrostatic interactions include repulsive charge-charge, dipole-dipole and charge-dipole interactions. Thus, the chemical composition and positions of charged, hydrophilic, and hydrophobic moieties within the fascin binding sites can be evaluated and compared to those of the test agent. As described above, the primary actin binding site of fascin include fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250. Moreover, fascin amino acid residues His392, Glu391, Ala488, Lys471, His474 and Asp473 form portions of the migrastatin analog binding site.

Specific computer software is available in the art to evaluate compound deformation energy and electrostatic interactions. Thus, for example, the test agents can be evaluated using such programs as: Gaussian 94, revision C (M. J. Frisch, Gaussian, Inc., Pittsburgh, Pa., 1995); AMBER, version 4.1 (P. A. Kollman, University of California at San Francisco, 1995); QUANTA/CHARMM (Molecular Simulations, Inc., San Diego, Calif. 01995); Insight II/Discover (Molecular Simulations, Inc, San Diego, Calif.® 1995); DelPhi (Molecular Simulations, Inc., San Diego, Calif. 1995); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach is the computational screening of small molecule databases for test agents that can bind in whole, or in part, to a fascin binding site. In this screening, the quality of fit of such entities to the binding site may be judged either by shape complementarity or by estimated interaction energy [E. C. Meng et al., J. Comp. Chem., 13, pp. 505-524 (1992)].

Therefore, one aspect of this invention is a machine-readable data storage medium, comprising a data storage material encoded with machine readable data which, when used by a machine programmed with instructions for using said data, displays a graphical three-dimensional representation of a molecule or molecular complex comprising a binding site defined by structure coordinates of fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250 (actin binding site) according to Table 2, or a homolog of said molecule or molecular complex, wherein said homolog comprises a binding site that has a root mean square deviation from the backbone atoms of said amino acids of not more than 1.5 angstroms.

Another aspect of the invention, is a machine-readable data storage medium, comprising a data storage material encoded with machine readable data which, when used by a machine programmed with instructions for using said data, displays a graphical three-dimensional representation of a molecule or molecular complex comprising a binding site defined by structure coordinates of fascin amino acid residues His392, Glu391, Ala488, Lys471, His474 and Asp473 (portions of the migrastatin analog binding site) according to Table 2, or a homolog of said molecule or molecular complex, wherein said homolog comprises a binding site that has a root mean square deviation from the backbone atoms of said amino acids of not more than 1.5 angstroms.

Preferably, the machine readable data, when used by a machine programmed with instructions for using said data, displays a graphical three-dimensional representation of a molecule or molecular complex comprising a binding site defined by structure coordinates fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250 (actin binding site) or by the structure coordinates of fascin amino acid residues His392, Glu391, Ala488, Lys471, His474 and Asp473 (portions of the migrastatin analog binding site) according to Table 2, or a homolog of said molecule or molecular complex, wherein said homolog comprises a binding pocket that has a root mean square deviation from the backbone atoms of said amino acids of not more than 1.5 angstroms.

In another embodiment, the machine-readable data storage medium comprises a data storage material encoded with a first set of machine readable data which comprises the Fourier transform of the structure coordinates set forth in Table 2, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

For example, the Fourier transform of the structure coordinates set forth in Table 2 may be used to determine at least a portion of the structure coordinates of other fascins, such as fascin 2, fascin 3, fascin homolog 1 and isoforms of fascin 2, fascin 3, fascin homolog 1.

FIG. 15 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

Another aspect of the invention is a computer for producing a three-dimensional representation of a molecule or molecular complex, wherein said molecule or molecular complex comprises a binding site defined by fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250 (actin binding site) or by fascin amino acid residues His392, Glu391, Ala488, Lys471, His474 and Asp473 (portions of the migrastatin analog binding site) according to Table 2, or a homolog of said molecule or molecular complex, wherein said homolog comprises a binding site that has a root mean square deviation from the backbone atoms of said amino acids of not more than 1.5 angstroms, wherein said computer comprises: (a) a machine readable data storage medium comprising a data storage material encoded with machine-readable data, wherein said machine readable data comprises the structure coordinates of fascin or portions thereof; (b) a working memory for storing instructions for processing said machine-readable data; (c) a central-processing unit coupled to said working memory and to said machine-readable data storage medium, for processing said machine-readable data into said three-dimensional representation; and (d) an output hardware coupled to said central processing unit, for receiving said three dimensional representation.

In some embodiments, the computer produces a three-dimensional representation of a molecule or molecular complex of an actin binding site, wherein said molecule or molecular complex comprises a binding pocket defined by the structural coordinates of fascin amino acid residues Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250 (actin binding site) or by the structure coordinates of fascin amino acid residues His392, Glu391, Ala488, Lys471, His474 and Asp473 (portions of the migrastatin analog binding site) according to Table 2, or a homolog of said molecule or molecular complex, wherein said homolog comprises a binding pocket that has a root mean square deviation from the backbone atoms of said amino acids of not more than 1.5 angstroms.

In some embodiments, the structure of a fascin polypeptide fragment can used for generating such a three-dimensional representation, where the fascin polypeptide fragment includes the actin binding site and/or the migrastatin analog binding site, e.g., any of SEQ ID NO:9-12.

FIG. 16 shows a cross section of a magnetic data storage medium 100 which can be encoded with a machine-readable data that can be carried out by a system such as system 10 of FIG. 15. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in manner which may be conventional, machine readable data such as that described herein, for execution by a system such as system 10 of FIG. 15.

FIG. 17 shows a cross section of an optically-readable data storage medium 110 which also can be encoded with such a machine-readable data, or set of instructions, which can be carried out by a system such as system 10 of FIG. 15. Medium 110 can be a conventional compact disk read only memory (CD-ROM) or a rewritable medium such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

Thus, in accordance with the present invention, data capable of displaying the three dimensional structure of fascin and portions thereof and their structurally similar homologues is stored in a machine-readable storage medium, which is capable of displaying a graphical three-dimensional representation of the structure.

Thus, the fascin X-ray coordinate data, for example, when used in conjunction with a computer programmed with software to translate those coordinates into the 3-dimensional structure of fascin, can be used for a variety of purposes, such as drug discovery.

Methods for identifying test agents that interact with fascin, where the physical interaction is detected, are also encompassed by the invention. Test agents can be screened and likely candidates can be identified by biological assays and binding assays. Moreover, the candidate inhibitors identified using the computer assisted structural design methods described above can be further tested and screened for useful biological activities using such biological assays and binding assays.

Binding assays between fascin and test agents may be carried out in several formats, including cell-based binding assays, solution-phase assays, solid phase based assays and immunoassays. In general, test agents are incubated with fascin for a specified period of time followed by measurement of binding between the tumor-specific protease and the test sample or compound. A label or reporter molecule attached to the fascin or a test agent can be employed, which is detectable by microscopy, fluorimetry, a scintillation counter, an enzyme or any available immunoassay.

In general, an assay for identifying compounds or molecules that interact with fascin involves incubating the fascin with a test sample that may contain such a compound or molecule under conditions that permit binding of the compound or molecule to the fascin, and measuring whether binding has occurred. Fascin may be purified or present in mixtures, such as in cultured cells, tissue samples, body fluids, culture medium or an aqueous in vitro solution. Assays can be used that are qualitative or quantitative. Quantitative assays can be used for determining the binding parameters (affinity constants and kinetics) of the test agent or candidate fascin inhibitor for fascin. Assays may also be used to evaluate the binding of a test agent to fascin fragments, fascin domains (e.g., the fascin actin binding domain or the fascin migrastatin analog binding domain).

The test agent may be substantially purified or present in a crude mixture. Test agents can be nucleic acids, proteins, peptides, carbohydrates, lipids or small molecular weight organic compounds. The test agents can be further characterized by their ability to increase or decrease fascin activity in order to determine whether they stimulate or inhibit fascin activity.

For example, fascin affinity assays can be performed where fascin is bound to a solid substrate and the bound fascin is exposed to individual test agents or mixtures of test agents. Test agents that bind to the fascin are candidate fascin modulating agents. The solid substrate can be any convenient solid surface such as a bead, microtiter well, or column matrix. Test agents can also be separately incubated with fascin and the fascin-test agent mixture electrophoretically separated under mild, non-denaturing conditions. When a test agent binds to fascin the apparent molecular weight of the fascin-test agent complex will be greater than the molecular weight of fascin alone. Such a shift in molecular weight can readily be visualized by staining the electrophoretically separated mixtures (e.g., in a polyacrylamide gel). Test agents can also be screened to ascertain whether they competitively inhibit actin binding or binding of migrastatin analogs to fascin. In such a competitive binding assay, the amount of actin bound to fascin can be quantified, for example, by observing how much labeled actin remains associated or bound to fascin after exposure and incubation with a test agent. Thus, for example, binding can be detected by labeling actin a competitive radioimmunoassay.

These and other procedures that are readily available to those of skill in the art can be employed to identify agents that can bind to fascin.

When evidence exists that a test agent can bind to fascin, that test agent can be further tested in biological assays to determine whether it can inhibit the activity of fascin. Alternatively, biological assays can be used to screen for useful fascin modulating agents. As described herein, fascin facilitates actin bundling. Thus, test agents can be screened to ascertain whether they inhibit actin bundling by fascin using, for example, the F-actin pelleting assay described herein (or that described by Yamashiro-Matsumura et al. 1985). Such an assay involves low-speed centrifugation where the actin bundles are pelleted. For example, as shown in FIG. 6A, addition of purified fascin to F-actin increased the amounts of F-actin bundles in the pellets. Test agents that inhibit such actin bundling are candidate fascin inhibitors or modulating agents.

While fascin may be involved in the prognosis of a variety of diseases, metastasis of cancer is one of the more significant diseases in which fascin plays a role. One method of screening whether test agents and/or candidate fascin inhibitors have useful anti-metastasis activity is the Boyden Chamber Cell Migration Assay, which involves an upper and a lower set of wells separated by a cell-permeable membrane. Cells (typically cancer cells) are suspended in one chamber and a chemoattractant can be present in a lower chamber. The test agent can be placed in the upper chamber or in both chambers. Cells will migrate through the membrane to the lower chamber if the test agent does not inhibit such migration (e.g., because the test agent inhibits fascin bundling of actin). The Example of this application further illustrate and describe this type of assay.

Further assays can be performed to assess the in vivo toxicity and in vivo efficacy of a test agent or drug candidate for treating disease (e.g. cancer). Suitable animal models and tumor cell lines can be used for these purposes. For example, mice, rats or other model animals with a propensity for developing cancer can be employed. Alternatively, small tumors or tumor cells or cancer cells that are known to metastasize can be transplanted into the model animals. The tumor or cancer cells can be treated with the test agent prior to transplantation. Alternatively, some of the animals that received tumors, tumor cells or cells then treated with the test agent or candidate fascin inhibitor. Other of those animals are control animals and/or are treated with a control agent. Tumor growth and physical signs can be monitored daily including any gross evidence of tumor necrosis, local tumor ulceration as well as evidence of toxicity including mobility, response to stimulus, eating, and weight of each animal. Test agents or candidate inhibitors that effectively reduce or eliminate tumors while having minimal negative effects on the health, lifespan and tissue integrity of the model animal are selected for development as chemotherapeutic agents and/or inhibitors of metastasis.

Assays may be used to identify agents that can interact with a cancer cell of interest. A wide variety of assays may be used for this purpose. See, for example, the assays carried out within the National Cancer Institute's “In Vitro Cell Line Screening Project.” In general, such an assay can involve contacting a cancer cell of interest with at least one agent and observing whether the agent kills the cancer cell and/or has other deleterious effects upon that cell.

Pluralities of assays can be performed in parallel with different test agents or candidate fascin inhibitors at different concentrations to obtain a differential response to the various concentrations. Typically, at least one control assay is included in the testing. Such a control can be a negative control involving exposure of the cancer cells of interest to a physiologic solution containing no agents. Another control can involve exposure of the cancer cell of interest to an agent that has already been observed to adversely affect the cancer cell of interest, or a second cell that is related to the cell of interest. Another control can involve exposing a cell of interest to a known therapeutic compound that has a desired effect on the cancer cell of interest, for example, an anti-cancer agent with known efficacy at a particular concentration or dosage. One of skill in the art can readily select control compounds and conditions that facilitate screening and analysis of the effects of the cyclic peptides on a cancer cell of interest.

Any cell type can be assayed by these methods. For example, any mammalian or other animal cancer cell type can be screened to assess whether the agents of the invention can selectively interact therewith. Mammalian or other animal cells can also be screened to ascertain whether the agents of the invention selectively interact therewith and/or to determine whether the agents of the invention do not interact, bind, lyse, kill or otherwise adversely affect the viability of the mammalian or other animal cell.

Conditions for screening include conditions that are used by one of skill in the art to grow, maintain or otherwise culture cell types of interest. Cancer cell types of interest should be assayed under conditions where they would be healthy but for the presence of the agents. Controls can be performed where the cell types are maintained under the selected culture conditions and not exposed to an agent, to assess whether the culture conditions influenced the viability of the cells. One of skill in the art can also perform the assay on cells that have been washed in simple physiological solutions, such as buffered saline, to eliminate, or test for, any interaction between the agents or cells and the components in the culture media. However, culture conditions for the assays generally include providing the cells with the appropriate concentration of nutrients, physiological salts, buffers and other components typically used to culture or maintain cells of the selected type. A variety of other reagents may be included in the screening assay. These include reagents like salts, neutral proteins, albumin, and serum (e.g. fetal calf serum) that are used to mimic the physiologic state of the cell types of interest. Conditions and media for culturing, growing and maintaining cells are available to one of skill in the art.

The selected reagents and components are added to the assay in the order selected by one of skill in the art. In general, the agents are added last to start the assay. Assays are performed at any suitable temperature, typically between 4° C. and 40° C. For example, the temperature may generally range from about room temperature (about 20° C.) to about 37° C. Incubation periods are selected to ascertain the optimal range of activity, or to insure that the test agents do not adversely affect normal, non-cancerous cells. However, incubation times can be optimized to facilitate rapid high-throughput screening. Typically, incubation times are between about one minute and about five days, for example, from about 30 minutes to about 3 days.

Test agents having the desired activity in vitro may be tested for activity and/or lack of toxicity in vivo, in an appropriate animal model. Such animal models include primates as well as mice, rats, rabbits, cats, dogs, pigs, goats, cattle or horses. For example, the mouse is a convenient animal model for testing whether agents of the invention have toxic effects and/or to determine whether the agents can inhibit metastasis of a cancer cell.

One of skill in the art can readily perform in vivo evaluation of the agents of the invention. For toxicity testing, a series of test agents at different test dosages can be separately administered to different animals. A single dose or, a series of dosages can be administered to the animal. A test period is selected that permits assessment of the effects of the agent(s) on the animal. Such a test period can run from about one day to about several weeks or months.

The effect of a agent(s) on an animal can be determined by observing whether the agent adversely affects the behavior (e.g., lethargy, hyperactivity) and physiological state of the animal over the course of test period. The physiological state of the animal can be assessed by standard procedures. For example, during the test period one of skill in the art can draw blood and collect other bodily fluids to test, for example, for various enzymes, proteins, metabolites, and the like. One of skill in the art can also observe whether the animal has bloating, loss of appetite, diarrhea, vomiting, blood in the urine, loss of consciousness, and a variety of other physiological problems. After the test period, the animal can be sacrificed and anatomical, pathological, histological and other studies can be performed on the tissues or organs of the animal.

For example, to determine whether one or more test agents can inhibit cancer cell metastasis, mice are infected with the selected cancer and a selected test dosage of one or more test agents is administered shortly thereafter. Alternatively, the tumor cells can be treated with the test agent prior to transplantation of the cells into the mice. Mice are observed over the course of several days to several weeks to ascertain whether the agents protect the mice from metastasis of cancer cells. At the end of the test period, mice can be sacrificed and examined to ascertain whether the agent has optimally protected the mice from metastasis and/or to determine whether any adverse side effects have occurred.

Controls are used to establish the effects of the cancer when the agent is not administered. Other controls can also be performed, for example, to determine the safety and efficacy of the present agents compared to that of known anti-cancer compounds and inhibitors of metastasis.

### Methods of Use

Agents that modulate the activity of fascin can be used to treat a variety of diseases and conditions. For example, as illustrated herein, fascin promotes actin bundling and plays a key role in cell migration and metastasis of cancer cells. Hence, modulators and inhibitors of fascin can be used to treat and inhibit metastatic cancer, including the compounds, migrastatin analogs, inhibitory nucleic acids, anti-fascin antibodies, test agents and candidate fascin modulators described herein.

However, fascin also plays a role in other diseases and conditions. For example, neurite shape and trajectory is modulated by fascin. Kraft et al., Phenotypes of Drosophila brain neurons in primary culture reveal a role for fascin in neurite shape and trajectory. J. NEUROSCI. (2006). Fascin is also involved in neuronal degeneration. Fulga et al., Abnormal bundling and accumulation of F-actin mediates tau-induced neuronal degeneration in vivo. NAT CELL BIOL. 9(2):139-48 (2007). In addition, fascin plays a role in Hodgkin's disease. Pinkus et al., Fascin, a sensitive new marker for Reed-Sternberg cells of Hodgkin's disease. Evidence for a dendritic or B cell derivation? AM. J. PATHOL. (1997). Fascin also plays a role in processing and presenting antigens, for example, on antigen presenting cells. Mosialos et al., Circulating human dendritic cells differentially express high levels of a 55-kd actin-bundling protein. AM. J. PATHOL. 148(2): 593-600 (1996); Pinkus et al., The role of follicular and interdigitating dendritic cells in HIV-related lymphoid hyperplasia: localization of fascin. Mod Pathol. 10(5):421-27 (1997). Moreover, fascin also plays a role in ischemic injury. Meller et al., Ubiquitin proteasome-mediated synaptic reorganization: a novel mechanism underlying rapid ischemic tolerance. J. Neurosci. 28(1):50-9 (2008).

According to the invention, agents that modulate fascin activity (e.g., the compounds, fascin polypeptide fragments, antibodies and inhibitory nucleic acid described herein) can be used for treating and inhibiting metastatic cancer, neuronal disorders, neuronal degeneration, inflammatory conditions, viral infections, bacterial infections, lymphoid hyperplasia, Hodgkin's disease, and ischemia-related tissue damage.

Tumor metastasis is the major cause of death of cancer patients (Weiss 2000, Fidler 2003). Thus, inhibition or prevention of tumor metastasis will significantly increase the survival rate of cancer patients, allow more moderate radiation or chemotherapy with less side-effects, and control the progression of solid tumors.

Tumor cell migration and invasion are critical steps in the process of tumor metastasis (Partin et al. 1989, Aznavoorian et al. 1993, Condeelis et al. 2005). For cell migration to proceed, the actin cytoskeleton must be reorganized by forming polymers and bundles to affect the dynamic changes of cell shapes (Jaffe et al. 2005, Matsudaira 1994, Otto 1994). Individual actin filaments are flexible and elongation of individual filaments per se is insufficient for membrane protrusion which is necessary for cell migration. Bundling of actin filaments provides rigidity to actin filaments for protrusion against the compressive force from the plasma membrane (Mogilner et al. 2005).

One of the critical actin-bundling proteins is fascin. Fascin is the primary actin cross-linker in filopodia, which are membrane protrusions critical for the migration and metastasis of cancer cells. Fascin is required to maximally cross-link the actin filaments into straight, compact, and rigid bundles. Elevated expressions of fascin mRNA and protein in cancer cells have been correlated with aggressive clinical course, poor prognosis and shorter survival.

According to the invention, metastatic cancer can be treated, prevented and/or inhibited by administering fascin inhibitors.

As used herein, the term “cancer” includes solid mammalian tumors as well as hematological malignancies. The terms “tumor cell(s)” and “cancer cell(s)” are used interchangeably herein.

“Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin.

The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS.

In addition, a cancer at any stage of progression can be treated, such as primary, metastatic, and recurrent cancers. In some embodiments, cancers are treated before metastasis is detected, for example, to inhibit metastatic cancer from developing. In other embodiments, cancers are treated when metastasis is detected, for example, to inhibit further metastasis and progression of the cancer.

The invention can also be used to treat autoimmune deficiency syndrome-associated Kaposi's sarcoma, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc.

As used herein the terms “normal mammalian cell” and “normal animal cell” are defined as a cell that is growing under normal growth control mechanisms (e.g., genetic control) and that displays normal cellular differentiation and normal migration patterns. Cancer cells differ from normal cells in their growth patterns, migration and in the nature of their cell surfaces. For example cancer cells tend to grow continuously and chaotically, without regard for their neighbors, and can migrate to distal sites to generate tumors in other areas of the body (i.e., metastasize).

The present invention is directed, in some embodiments, to methods of treating or inhibiting metastatic cancer in an animal, for example, for human and veterinary uses, which include administering to a subject animal (e.g., a human), a therapeutically effective amount of an agent (e.g. a migrastatin analog, an inhibitory nucleic acid or an anti-fascin antibody) of the present invention.

Treatment of, or treating, a disease (e.g., cancer) is intended to include the alleviation of or diminishment of at least one symptom typically associated with the disease. The treatment also includes alleviation or diminishment of more than one symptom. The treatment may cure the disease, for example, by eliminating the symptoms and/or the source of the disease or condition. For example, treatment can cure the cancer by substantially inhibiting metastasis of the cancer cells so that removal or killing of the primary tumor or cancer cell(s) substantially eliminates the cancer. Treatment can also arrest or inhibit the metastasis of the cancer and/or tumor cells without directly killing or promoting the apoptosis of cancer cells.

Fascin functions in a variety of cellular functions that play critical roles in modulating the growth, movement and interaction of cells. However the actin bundling function of fascin is directly involved in tumor metastasis and invasive growth.

The anti-metastatic activity of fascin (e.g., in the presence of various test agents or therapeutic agents like those described herein) can be evaluated against varieties of cancers using methods described herein and available to one of skill in the art. Anti-cancer activity, for example, can be determined by identifying the dose that inhibits 50% cancer cell metastasis (GI50) of an agent of the invention.

The present invention also provides a method of evaluating a therapeutically effective dosage for treating a cancer (e.g., inhibiting metastasis) with an agent of the invention that includes determining the GI50 of the agent in vitro. Such a method permits calculation of the approximate amount of agent needed per volume to inhibit cancer cell migration. Such amounts can be determined, for example, by standard microdilution methods.

In some embodiments, the agents of the invention can be administered in multiple doses over an extended period of time, or intermittently.

The term ‘animal,’ as used herein, refers to an animal, such as a warm-blooded animal, which is susceptible to or has a disease associated with fascin activity or expression, for example, metastatic cancer. Mammals include cattle, buffalo, sheep, goats, pigs, horses, dogs, cats, rats, rabbits, mice, and humans. Also included are other livestock, domesticated animals and captive animals. The term ‘farm animals’ includes chickens, turkeys, fish, and other farmed animals. Mammals and other animals including birds may be treated by the methods and compositions described and claimed herein.

### Formulation and Administration

The compounds of the invention, including the compounds, migrastatin analogs, inhibitory nucleic acids, anti-fascin antibodies, test agents and candidate fascin modulators described herein, can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Inhibitory nucleic acids can be introduced into cells by a number of methods. In lipid-mediated transfection, cells take in non-covalent complexes between nucleic acid and a lipid or polymer reagent by endocytosis. Electroporation utilizes a brief electrical pulse to cause disruptions or holes in the cells' plasma membrane through which nucleic acid enters. Both of these methods successfully deliver any of the RNAi nucleic acids except viral vectors. Viral vector delivery occurs by infection of cells with the corresponding virus generated via a multi-step process. Viral vectors lack the ability to replicate themselves. Specialized cells express the missing genes necessary for viral replication and packaging. These cells produce and release virus into the culture medium upon conventional transfection with the viral vector. The virus containing the viral vector is collected and purified. Infection of the desired cell line with virus introduces the siRNA or shRNA and knocks down gene expression. The viral delivery method absolutely requires the use of viral vectors and cannot accommodate the other sources of nucleic acid for RNAi.

Delivery of siRNA can be carried out by direct delivery of naked siRNA; encapsulation into liposomes and lipoplexes; conjugation to antibodies, peptides, aptamers, and other molecules; and formation of complexes with chemical and biological polymers. Intravenous, intraperetoneal, intranasal, and intratumoral siRNA administration can be carried out using polymer carriers and nanoparticles including PEI, low molecular weight PEI, chitosan, atelocollagen, transferrin targeted nanoparticles, liquid-targeted stabilized nanoparticles and dynamic polyconjugates.

Delivery of siRNA can further be carried out by conjugation of siRNA molecules to a targeting molecule including but not limited to proteins, peptides, and aptamers. In the case of peptides, a basic region, such as a poly-Arg stretch, is used (Kumar P, et al. Nature 2007 Jul. 5; 448(7149):39-43; Kim W J, et al. Mol Ther 2006 September; 14(3):343-350). For antibodies, conjugation to a protamine fusion protein can be used (Song E, et al. Nat Biotechnol 2005 June; 23(6):709-717).

The present compounds including migrastatin analogs and inhibitory nucleic acids may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compounds described herein may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with several of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the invention in a liquid composition, such as a lotion, will be from about 0.01-25 wt-%, preferably from about 0.1-10 wt-%. The concentration in a semi-solid or solid composition such as a gel or a powder will be about 0.01-10 wt-%, preferably about 0.1-5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 1.0 to about 200 mg/kg, e.g., from about 1 to about 100 mg/kg of body weight per day, such as about 2.0 to about 100 mg/kg of body weight per day, such as about 3.0 to about 50 mg per kilogram body weight of the recipient per day, preferably in the range of about 5 to 20 mg/kg/day. Alternatively, the compositions can be administered five times a week on five consecutive days with a two day rest, or four times a week on four consecutive days with a three day rest, or every other day.

Methods for extrapolating effective dosages in mice and other animals, to humans are known in the art (See, for example, U.S. Pat. No. 4,938,949). For example, in certain embodiments, compounds of the invention (for example those useful for the treatment of colon and/or ovarian cancer) may be administered at dosage levels of about 0.01 mg/kg to about 300 mg/kg, from about 0.1 mg/kg to about 250 mg/kg, from about 1 mg/kg to about 200 mg/kg, from about 1 mg/kg to about 150 mg/kg, from about 1 mg/kg to about 100 mg/kg, from about 1 mg/kg to about 90 mg/kg, from about 1 mg/kg to about 80 mg/kg, from about 1 mg/kg to about 70 mg/kg, from about 1 mg/kg to about 60 mg/kg, from about 1 mg/kg to about 50 mg/kg, from about 1 mg/kg to about 40 mg/kg, from about 1 mg/kg to about 30 mg/kg, from about 1 mg/kg to about 20 mg/kg, from about 5 mg/kg to about 100 mg/kg, from about 5 mg/kg to about 90 mg/kg, from about 5 mg/kg to about 80 mg/kg, from about 5 mg/kg to about 70 mg/kg, from about 5 mg/kg to about 60 mg/kg, from about 5 mg/kg to about 50 mg/kg, from about 5 mg/kg to about 40 mg/kg, from about 5 mg/kg to about 30 mg/kg, from about 5 mg/kg to about 20 mg/kg, from about 10 mg/kg to about 100 mg/kg, from about 10 mg/kg to about 90 mg/kg, from about 10 mg/kg to about 80 mg/kg, from about 10 mg/kg to about 70 mg/kg, from about 10 mg/kg to about 60 mg/kg, from about 10 mg/kg to about 50 mg/kg, from about 10 mg/kg to about 40 mg/kg, from about 10 mg/kg to about 30 mg/kg, from about 10 mg/kg to about 20 mg/kg, from about 20 mg/kg to about 100 mg/kg, from about 20 mg/kg to about 90 mg/kg, from about 20 mg/kg to about 80 mg/kg, from about 20 mg/kg to about 70 mg/kg, from about 20 mg/kg to about 60 mg/kg, from about 20 mg/kg to about 50 mg/kg, from about 20 mg/kg to about 40 mg/kg, from about 20 mg/kg to about 30 mg/kg, of subject body weight per day, one or more times a day, to obtain the desired therapeutic effect. In certain embodiments, compounds may be administered at a dosage of about 1 mg/kg or greater, 5 mg/kg or greater; 10 mg/kg or greater, 15 mg/kg or greater, 20 mg/kg or greater, 25 mg/kg or greater, 30 mg/kg or greater, 35 mg/kg or greater, 40 mg/kg or greater, 45 mg/kg or greater, 50 mg/kg or greater, 60 mg/kg or greater, 70 mg/kg or greater, of body weight. It will also be appreciated that dosages smaller than 0.01 mg/kg or greater than 70 mg/kg (for example 70-200 mg/kg) can be administered to a subject.

In certain embodiments, compounds may be used in chemotherapy (i.e., to inhibit metastasis) and may be administered at higher dosage. For example, compounds to be used in chemotherapy may be administered from about 100 mg/kg to about 300 mg/kg, from about 120 mg/kg to about 280 mg/kg, from about 140 mg/kg to about 260 mg/kg, from about 150 mg/kg to about 250 mg/kg, from about 160 mg/kg to about 240 mg/kg, of subject body weight per day, one or more times a day, to obtain the desired therapeutic effect.

In certain other embodiments, compounds may be used in supportive therapy (e.g., as an adjuvant to surgery or irradiation in a range of common types of tumor) and may be administered at lower dosage. For example, compounds to be used in supportive therapy may be administered from about 1 mg/kg to about 30 mg/kg, from about 1 mg/kg to about 25 mg/kg, from about 5 mg/kg to about 20 mg/kg, of subject body weight per day, one or more times a day, to obtain the desired therapeutic effect.

In certain other embodiments, compounds may be used for preventing and/or treating metastatic cancer (e.g., ovarian and/or colon cancer) and may be administered at an intermediate dosage. For example, compounds to be used in supportive therapy may be administered from about 1 mg/kg to about 100 mg/kg, from about 1 mg/kg to about 80 mg/kg, from about 5 mg/kg to about 70 mg/kg, from about 10 mg/kg to about 70 mg/kg, from about 10 mg/kg to about 60 mg/kg, from about 20 mg/kg to about 70 mg/kg, from about 20 mg/kg to about 60 mg/kg, of subject body weight per day, one or more times a day, to obtain the desired therapeutic effect.

The compound is conveniently administered in unit dosage form; for example, containing 45 to 3000 mg, conveniently 90 to 2250 mg, most conveniently, 450 to 1500 mg of active ingredient per unit dosage form. In some embodiments, the compound is administered at dosages of about 1 to about 100 mg/kg.

Ideally, the active ingredient should be administered to achieve peak plasma concentrations of the active compound of from about 0.5 nM to about 10 μM, preferably, about 1 nM to 1 μM, most preferably, about 10 nM to about 0.5 μM. This may be achieved, for example, by the intravenous injection of a 0.05 to 5% solution of the active ingredient, optionally in saline, or orally administered as a bolus containing about 20-2000 mg of the active ingredient. Desirable blood levels may be maintained by continuous infusion to provide about 0.2 to 1.0 mg/kg/hr or by intermittent infusions containing about 0.4 to 20 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention are useful as therapeutic agents administered for inhibition of cell migration and treatment of metastatic cancer. Such cancers include but are not limited to, cancers involving the animal's head, neck, lung, mesothelioma, mediastinum, esophagus, stomach, pancreas, hepatobiliary system, small intestine, colon, colorectal, rectum, anus, kidney, ureter, bladder, prostate, urethra, penis, testis, gynecological organs, ovaries, breast, endocrine system, skin, or central nervous system. Thus, for example, the cancer can be a breast cancer, a leukemia, a lung cancer, a colon cancer, a central nervous system cancer, a melanoma, an ovarian cancer, a renal cancer, or a prostate cancer.

Additionally, compounds of the invention may be useful as pharmacological tools for the further investigation of the inhibition of cell migration.

The compounds of the invention can also be administered in combination with other therapeutic agents that are effective for treating or controlling the spread of cancerous cells or tumor cells.

Moreover, the compounds of the invention can be tested in appropriate animal models. For example, the compounds of the invention can be tested in animals with known tumors, or animals that have been injected with tumor cells into a localized area. The degree or number of secondary tumors that form over time is a measure of metastasis and the ability of the compounds to inhibit such metastasis can be evaluated relative to control animals that have the primary tumor but receive no test compounds. Experimental results from this type of in vivo testing are shown in FIG. 8 and are further described in the Examples. These results demonstrate that the compounds of the invention substantially reduce or eliminate tumor metastasis.

The compounds of the invention will also find use in treatment of brain disorders (Kraft et al., Phenotypes of Drosophila brain neurons in primary culture reveal a role for fascin in neurite shape and trajectory. J. Neurosci. (2006)); Hodgkin's disease (Pinkus et al., Fascin, a sensitive new marker for Reed-Sternberg cells of Hodgkin's disease. Evidence for a dendritic or B cell derivation? Am. J. Pathol. (1997)); virus infection (Mosialos et al., Circulating human dendritic cells differentially express high levels of a 55-kd actin-bundling protein. Am. J. Pathol. (1996)); neuronal degeneration (Fulga et al., Abnormal bundling and accumulation of F-actin mediates tau-induced neuronal degeneration in vivo. Nat Cell Biol. 2007 February; 9(2):139-48)); lymphoid hyperplasia (Said et al., The role of follicular and interdigitating dendritic cells in HIV-related lymphoid hyperplasia: localization of fascin. Mod Pathol. 1997 May; 10(5):421-7)); and ischemia (Meller et al., Ubiquitin proteasome-mediated synaptic reorganization: a novel mechanism underlying rapid ischemic tolerance. J. Neurosci. 2008 Jan. 2; 28(1):50-9.))

The invention will now be illustrated by the following non-limiting Examples.

### Example 1

### Chemical Synthesis and Characterization

This Example describes the synthesis as well as the chemical and physical characterization of compounds.

Synthesis: Compounds of the invention can be synthesized as shown below.

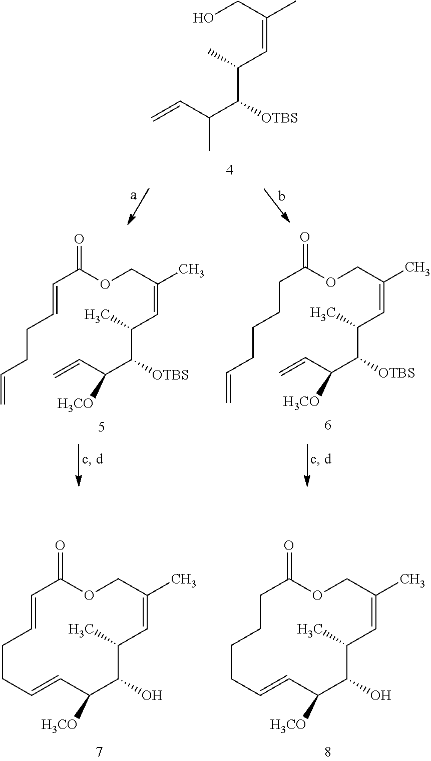

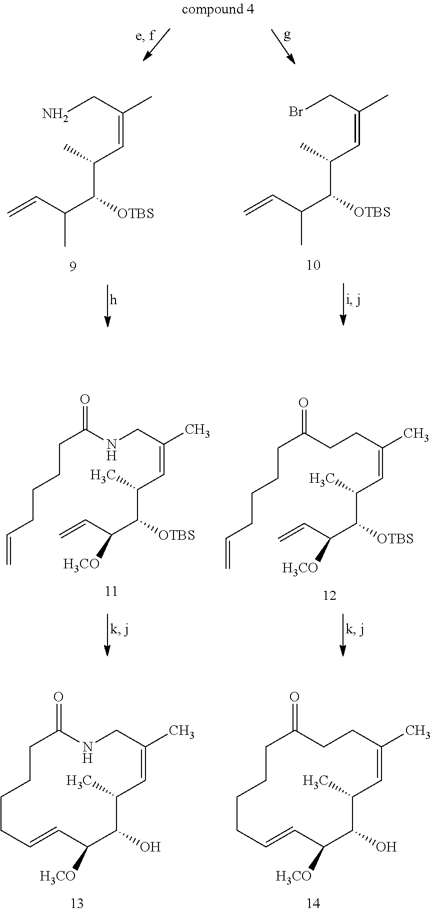

The reagents and conditions employed are as follows: (a) Yamaguchi acylation (48%); (b) Et₃N, DMAP, 6-heptenoyl chloride (89%); (c) Grubbs catalyst, toluene and reflux (47 and 73%); (d) HF-pyridine, THF (78 and 90%); (e) diphenylphosphoryl azide (87%); (f) PPh₃, H₂O (90%); (g) CBr₄, PPh₃ (95%); (h) EDCI, 6-heptenioc acid (70%); (i) 1-benzenesulfonyl-oct-7-en-one, DBU (75%); (j) Na/Hg (79%); (k) Grubbs catalyst, toluene, reflux (70 and 75%); (1) HF-pyridine, THF (90 and 95%).
Analytical Equipment: Optical rotations are measured on a JASCO DIP-370 digital polarimeter at room temperature. Concentration (c) in g/100 ml and solvent are given in parentheses. Infrared spectra are obtained on a Perkin-Elmer 1600 FT-IR spectrophotometer neat or as a film in CHCl₃(NaCl plates). Absorption bands are noted in cm⁻¹. ¹H- and ¹³C-NMR spectra are recorded on a Bruker AMX-400 MHz or a Bruker Advance DRX-500 MHz spectrometer in CDCl3 (referenced to 7.26 ppm (δ) for ¹H-NMR and 77.0 ppm for ¹³C-NMR). Coupling constants (J) (H,H) are given in Hz, spectral splitting patterns are designated as singlet (s), doublet (d), triplet (t), quadruplet (q), multiplet or more overlapping signals (m), apparent (app), broad signal (br). Low resolution mass spectra (ionspray, a variation of electrospray) are acquired on a Perkin-Elmer Sciex API 100 spectrometer. Samples are introduced by direct infusion. High resolution mass spectra (fast atom bombardment, FAB) are acquired on a Micromass 70-SE-4F spectrometer.

Migrastatin core 7: [α]_{D}+106.0° (c 0.50, CHCl3); IR (CHCl₃) 3567, 2933, 2881, 1716, 1602, 1448, 1393, 1255, 1107, 1052; ¹H-NMR (500 MHz, CDCl₃) δ 6.81-6.75 (m, 1H), 5.73 (d, J=15.9, 1H), 5.62-5.55 (m, 2H), 5.14 (dd, J=15.2, 6.8, 1H), 4.72 (d, J=15.6, 1H), 4.63 (d, J=15.6, 1H), 3.42-3.38 (m, 2H), 3.28 (s, 3H), 3.03-2.97 (m, 1H), 2.69 (br s, 1H), 2.47-2.38 (m, 2H), 2.32-2.18 (m, 2H), 1.68 (s, 3H), 0.88 (d, J=6.9, 3H); ¹³C-NMR (125 MHz, CDCl₃) δ 165.36, 149.52, 133.85, 129.79, 129.51, 127.50, 122.15, 84.62, 76.09, 65.40, 56.25, 32.20, 31.34, 29.99, 22.27, 12.66; MS (ESI) 303 [M+Na⁺]; HRMS (FAB) calcd. for C₁₆H₂₄O₄ [M+Na⁺]303.1571, found 303.1572.

2,3-Dihydro-migrastatin core 8: [α]_{D}+115.3° (c 1.00, CHCl₃); IR (CHCl₃) 3567, 3016, 2933, 2858, 1724, 1450, 1387, 1317, 1258, 1145, 1115, 979; ¹H-NMR (500 MHz, CDCl₃) δ 5.74-5.67 (m, 2H), 5.23 (dd, J=15.7, 7.7, 1H), 4.54 (d, J=13.1, 1H), 4.29 (d, J=13.1, 1H), 3.46-3.39 (m, 2H), 3.30 (s, 3H), 2.82-2.77 (m, 1H), 2.44-2.39 (m, 1H), 2.26-2.15 (m, 2H), 2.03-1.97 (m, 1H), 1.74 (d, J=0.9, 3H), 1.74-1.70 (m, 1H), 1.60-1.52 (m, 2H), 1.36-1.32 (m, 1H), 0.93 (d, J=6.9, 3H); ¹³C-NMR (125 MHz, CDCl₃) δ 173.69, 135.19, 134.39, 129.02, 127.14, 83.82, 75.91, 64.76, 56.34, 34.23, 32.06, 29.88, 27.20, 23.40, 23.27, 12.81; MS (ESI) 305 [M+Na⁺]; HRMS (FAB) calcd. for C₁₆H₂₆O₄ [M+Na⁺] 305.1719, found 305.1729.

Migrastatin lactam 13: [α]_{D}+101.3° (c 1.00, CHCl₃); IR (CHCl₃) 3566, 3444, 3021, 2936, 2828, 1658, 1504, 1478, 1398, 1229, 1088, 979; ¹H-NMR (500 MHz, CDCl₃) ä 5.79-5.73 (m, 1H), 5.66 (d, J=10.2, 1H), 5.24 (dd, J=15.8, 7.5, 1H), 5.12 (br s, 1H), 3.91 (dd, J=13.7, 4.1, 1H), 3.50-3.46 (m, 2H), 3.34-3.30 (m, 1H), 3.31 (s, 3H), 2.89 (br s, 1H), 2.56-2.52 (m, 1H), 2.32-2.25 (m, 2H), 2.16-2.11 (m, 1H), 1.96-1.89 (m, 1H), 1.77 (d, J=1.1, 3H), 1.73-1.51 (m, 3H), 1.37-1.32 (m, 1H), 0.94 (d, J=6.9, 3H); ¹³C-NMR (125 MHz, CDCl₃) δ 173.36, 135.52, 133.77, 129.89, 128.73, 83.21, 76.38, 56.45, 41.40, 35.95, 32.27, 29.86, 27.00, 24.82, 24.42, 13.03; MS (ESI) 304 [M+Na⁺]; HRMS (FAB) calcd. for C16H27NO3 [M+Na⁺] 304.1888, found 304.1889.

Migrastatin ketone (14): [α]_{D}+77.0° (c 0.5, CHCl₃); IR (neat) 3566, 3022, 3015, 2975, 2937, 2879, 1700, 1448, 1384, 1237, 1109, 1085, 979 cm-1; ¹H-NMR (500 MHz, CDCl₃) ä 5.72 (ddd, J=15.0, 8.5, 6.0, 1H), 5.37 (dd, J=10.0, 0.9 1H), 5.31 (dd, J=15.6, 7.8, 1H), 3.47 (t, J=8.5, 1H), 3.36 (dd, J=9.2, 1.2, 1H), 3.31 (s, 3H), 2.78 (br s, 1H), 2.51-2.45 (m, 2H), 2.37-2.32 (m, 2H), 2.26-2.16 (m, 5H), 1.69 (d, J=1.3, 3H), 1.69-1.59 (m, 2H), 1.53-1.50 (m, 2H), 0.95 (d, J=6.8, 3H); ¹³C-NMR (125 MHz, CDCl₃) δ 212.10, 135.23, 132.91, 130.26, 129.22, 83.69, 77.62, 56.45, 42.08, 40.67, 32.57, 30.33, 28.57, 27.01, 23.22, 23.14, 12.61; MS (ESI) 303 [M+Na⁺]; HRMS (FAB) calcd. for C17H28O3Na [M+Na⁺] 303.1936, found 303.1938.

(R)-Isopropyl migrastatin (17): [α]_{D}+21.3° (c 0.09, CHCl₃); IR (neat) 3499, 2967, 2926, 2866, 1729, 1453, 1383, 1257, 1111, 981 cm-1; ¹H-NMR (500 MHz, CDCl₃) ä 5.65 (dt, J=15.5, 7.5, 1H), 5.58 (dd, J=10.7, 1.3, 1H), 5.35 (dd, J=15.5, 6.0, 1H), 4.87 (d, J=7.6, 1H), 3.49 (dd, J=9.1, 6.0, 1H), 3.34 (s, 3H), 3.27 (br d, J=8.8, 1H), 3.13-3.07 (m, 1H), 2.86, (br s, 1H), 2.34-2.15 (m, 4H), 2.06-1.99 (m, 1H), 1.76 (d, J=1.6, 3H), 1.75-1.58 (m, 3H), 1.47-1.41 (m, 1H), 0.98 (d, J=7.0, 3H), 0.93 (d, J=6.7, 3H), 0.92 (d, J=6.7, 3H); ¹³C-NMR (125 MHz, CDCl₃) δ 172.50, 132.45, 132.08, 131.58, 128.26, 82.45, 80.74, 77.44, 33.00, 32.66, 31.76, 30.56, 25.57, 24.91, 22.44, 19.02, 18.96, 13.20; MS (ESI) 324 [M+Na⁺]; HRMS (FAB) calcd. for C₁₉H₃₂O₄Na [M+Na⁺] 347.2198, found 347.2196.

(S)-Isopropyl migrastatin (18): [α]_{D}+25.1° (c 0.32, CHCl₃); IR (neat) 3479, 2967, 2926, 2876, 1724, 1448, 1373, 1257, 1237, 1091, 976 cm-1; ¹H-NMR (500 MHz, CDCl₃) ä 5.70 (ddd, J=15.4, 8.5, 5.3, 1H), 5.33 (dd, J=10.0, 0.9, 1H), 5.30 (d, J=7.0, 1H) 5.19-5.13 (m, 1H), 3.40-3.30 (m, 2H), 3.28 (s, 3H), 2.99-2.96 (m, 1H), 2.76 (s, 1H), 2.36-2.24 (m, 2H), 2.20-2.08 (m, 2H), 1.99 (dt, J=7.0, 6.9, 1H) 1.69 (d, J=1.3, 3H), 1.62-1.52 (m, 4H), 0.94 (d, J=7.0, 3H), 0.91 (d, J=6.6, 3H), 0.86 (d, J=6.9, 3H); ¹³C-NMR (125 MHz, CDCl₃) δ 172.97, 135.94, 133.83, 130.09, 127.75, 86.47, 78.70, 55.98, 33.99, 32.80, 30.38, 29.82, 27.34, 22.57, 21.38, 19.09, 18.05, 15.20; MS (ESI) 324 [M+Na⁺]; HRMS (FAB) calcd. for C19H32O4Na [M+Na⁺] 347.2198, found 347.2187.

### Example 2

### Inhibition of Metastatic Tumor Cell Migration by Migrastatin Analogs

The efficacy of the compounds of the invention for inhibiting cell migration was assessed using two procedures, a wound healing assay and a chamber cell migration assay.

### Methods

Cells. Mouse 4T1 mammary tumor cells and human MDA-MB-231 breast tumor cells were obtained from ATCC and have been described previously (Shan et al. 2005, Yang et al. 2005). 4T1 cells were cultured in RPMI 1640 medium supplemented with 10% FBS. MDA-MB-231 cells were cultured in DMEM supplemented with 10% FBS.
Wound-healing assay. The wound-healing assay involves observing whether confluent cells can migrate across a scrape or wound in the cell layer. Cell migration assays were performed as described previously (Yang et al. 2005, Shan et al 2006). Tumor cells were plated in a 24-well plate coated with gelatin in standard media. After the cells grew to confluence, wounds were made in the confluent layer of cell using a sterile instrument such as a sterile pipette tip. The cells were washed with Phosphate Buffered Saline (PBS) or other sterile solutions and then the migration was induced by adding medium supplemented with 10% FBS. When the wound for the positive control closed, cells were fixed with 3.7% formaldehyde and stained with crystal violet staining solution. Compounds that inhibit the migration of cells into the wound area at low concentrations are useful for inhibiting cell migration and treating metastatic cancer.
Chamber cell migration assay. The chamber cell migration assay assesses whether cell can migrate through a filter having pores of known sizes. For example, cell migrations can be assayed with Boyden chambers having filters with about 8.0 μm pore size. Briefly, cells in serum-free medium are added to the first chamber and 500 μl of medium with 10% fetal bovine serum (FBS) is added to the second chamber. The chamber is incubated for about 6-8 hours at 37° C. with different concentrations of chemical compounds in both of the two chambers. Cells in the first chamber are removed with a cotton swab, and cells in the other chamber or on the other side of the filter are fixed and stained. Photographs several random regions of the filter facing the second chamber are taken and the number of cells counted to calculate the average number of cells that had transmigrated.

### Results

The effects of the core macroketone and the core macrolactam analogs on the migration of tumor cells in vitro were studied. As shown in FIG. 1, while serum induced the migration of metastatic mouse breast tumor 4T1 cells, the addition of the macroketone or macrolactam congeners inhibited serum-induced 4T1 cell migration as measured by both the wound-healing assay and the Boyden chamber assay (FIG. 1B-D). The macroketone and macrolactam core structures were quite effective with IC₅₀ values of 100 and 255 nM, respectively (FIGS. 1C and 1D). The parent compound migrastatin had an IC50 of 29 μM (Njardarson et al. 2004, Gaul et al. 2004). These compounds had little effect on the proliferation of 4T1 cells in culture (Id.).

The macroketone and macrolactam congeners also inhibited the migration of several invasive and metastatic human tumor cell lines, such as human breast tumor MDA-MB 231 cells, human prostate tumor PC-3 cells, and human colon tumor Lovo cells (FIGS. 2A and B). In contrast, migration of normal human mammary gland epithelia MCF-10A cells, mouse embryonic fibroblast cells, or primary mouse leukocytes was rather insensitive to these compounds (FIGS. 2C and D). These cellular studies demonstrated that the macroketone and macrolactam core structures are highly selective for mouse and human metastatic tumor cells versus normal cells. These results also suggest that the level or activity of the biochemical target of these compounds might be high in metastatic tumor cells thus sensitizing these tumor cells to the compounds.

### Example 3

### Inhibition of Lung Metastasis of Highly Metastatic Mammary Carcinoma Cells by Migrastatin Analogs in Mice

The analogs were tested to determine if they could affect tumor metastasis in the 4T1 mouse mammary tumor model. The lung metastasis of 4T1 tumor cells in mice with or without treatment with these chemical compounds was examined. The mouse 4T1 tumor closely mimics human breast cancer in its anatomical site, immunogenicity, growth characteristics, and metastatic properties (Pulaski et al. 1998). From the mammary gland, the 4T1 tumor spontaneously metastasizes to a variety of target organs including the lung, bone, brain, and liver (Aslakson et al. 1992). Ten days after implantation of 4T1 cells (1×10⁵) in the mammary glands of BALB/c mice, the mice were injected intraperitoneally with the macroketone and the macrolactam core structures, or control saline PBS. The dosages of the macroketone or macrolactam core structures were 10 mg/kg or 20 mg/kg. The compounds were injected. After 20 days, the mice were sacrificed and metastasis to the lung was examined by clonogenic assay (Shen et al., 2005). While mice injected with the control saline (vehicle alone) showed large numbers of metastasized 4T1 cells in the lung, the number of metastasized 4T1 cells in the lungs of mice treated with either macroketone or macrolactam was reduced by 91%-99% (FIG. 3A). Mice treated with macroketone or macrolactam formed primary mammary tumors similar in size to those of mice treated with saline (FIG. 3C), implying that these chemical compounds did not interfere with primary tumor formation by 4T1 cells. These compounds did not cause obvious side effects since the mice appeared normal with no evidence of weight loss, lethargy, or ruffled fur. These results demonstrate that the macroketone and macrolactam are potent inhibitors of 4T1 tumor cell metastasis from the mammary gland to the lung.

As further controls for the specific effects of these core structures, two other compounds were examined: migrastatin semi-core and macrolactone (FIG. 3B). Upon testing with 4T1 cells for its ability to inhibit cell migration in vitro, migrastatin semi-core showed a significantly lower activity than macroketone and macrolactam with an IC₅₀ of 40 μM (Njardarson et al. 2004, Gaul et al. 2004). Although the macrolactone was very effective at inhibiting 4T1 cell migration (IC₅₀ of 24 nM), previous metabolic stability studies showed that it was very unstable in mouse plasma with a half-life of <5 minutes (Gaul et al. 2004). As shown in FIG. 3A, treatment of mice with migrastatin semi-core (10 and 20 mg/kg) did not significantly reduce the 4T1 tumor metastasis in these mice. Although the effect of 20 mg/kg macrolactone on 4T1 tumor metastasis was statistically significant, it was much less than those of macroketone and macrolactam (FIG. 3A). The reduced effectiveness of macrolactone was likely due to its instability in mice.

### Example 4

### Inhibition of Lamellipodium Formation by Migrastatin Analogs

The effects of macroketone and macrolactam on the actin cytoskeleton and microtubules in 4T1 cells was examined. Cell migration is a sequential and interrelated multi-step process (Ridley et al. 2003). It involves the formation of lamellipodia at the front edge, cycles of adhesion and detachment, cell body contraction, and tail retraction (Ridley et al. 2003). The core macroketone and the core macrolactam inhibited the formation of lamellipodia at the leading edge (Shan et al. 2005). While the addition of serum induced the formation of lamellipodia, addition of either the macroketone or macrolactam cores disrupted the formation of lamellipodia (Shan et al. 2005). Moreover, neither compound had any effect on the microtubule organization. These data demonstrated that the cellular basis of the action of these migrastatin analogs on tumor metastasis involves the disruption of actin cytoskeletal reorganization.

### Example 5

### Migrastatin Analogs Inhibit the Actin-Bundling Activity of Fascin

### Methods

Identification of fascin as the protein target of migrastatin analogs. Whole cell lysates from 4T1 mouse breast tumor cells were made. After preclearing the cell lysate with immobilized neutravidin biotin binding protein (Pierce, IL, USA) to remove biotin and avidin-binding proteins, the cell lysates were loaded to a column packed with the biotin-labeled macroketone (conjugated to neutravidin beads). A control column packed with free biotin and neutravidin beads was run side-by-side. After washing the column with 10 bed volumes of lysis buffer with 300 mM NaCl, the bound proteins were eluted with 0.1 M Glycine-HCl at pH 2.8 according to the manufacturer's instruction. From the SDS-PAGE, one band (˜55 kDa) was specifically present in the sample eluted from the biotin-labeled macroketone column but not in the sample eluted from the biotin column. The band containing this ˜55 kDa protein was cut out of the gel and the protein was identified as mouse fascin 1 by mass spectrometry.
Protein Expression and Purification. Recombinant GST-fascin fusion protein was produced in BL21 Escherichia coli. A 1-liter culture was grown to an A600 reading of 1.0 and then induced by addition of 0.3 mM isopropyl 1-thio-D-galactopyranoside (IPTG) for 12 hours at 25° C. Cells were flash frozen and then lysed by sonication in Tris-buffered saline. The supernatant was then incubated with glutathione-Sepharose for 2 h at 4° C. After extensive washing, GST-fascin was eluted and concentrated with a Centricon Plus-20 (Millipore). To remove the GST tag from the fusion protein, beads were incubated with thrombin overnight at 4° C. The supernatant was collected and concentrated.
GST-fascin and Biotin-macroketone Interaction. Purified recombinant fascin protein or control protein were incubated with biotin-macroketone for 2 h at 4° C. Proteins associated with biotin-macroketone were precipitated with Untralink-immobilized NeutrAvidin agarose (Pierce). After extensive washing, bound proteins were eluted with SDS sample buffer and resolved by 10% SDS-PAGE.
F-Actin Bundling Assay. Actin bundling activity was measured by low speed centrifugation assay and fluorescence microscopy. In low-speed centrifugation assay, monomeric rabbit G-actin was induced to polymerize at room temperature in F-actin buffer (20 mM Tris-HCl at pH 8, 1 mM ATP, 1 mM DTT, 2 mM MgCl2 and 100 mM KCl). Recombinant fascin proteins or control buffer were subsequently incubated with F-actin for 60 min at room temperature and centrifuged for 30 min at 10,000 g in an Eppendorf 5415D table-top centrifuge. Both supernatants and pellets were dissolved in an equivalent volume of SDS sample buffer, and the amount of actin was determined by SDS-PAGE. For fluorescence microscopy, monomeric G-actin was polymerized as described above. F-actin was mixed with recombinant fascin protein in F-buffer and incubated at room temperature for 30 min. Actin was then labeled by adding 5% rhodamine-phalloidine to the mixture. The samples were mounted between a slide and a coverslip coated with poly-lysine and imaged by fluorescence microscopy.
F-Actin Binding Assay. Actin polymerization was performed as described above. Recombinant fascin proteins or control buffer were subsequently incubated with F-actin for 60 min at room temperature. Mixtures were centrifuged at 100,000 g (Beckman Airfuge) for 30 min. Both supernatants and pellets were dissolved in an equivalent volume of SDS sample buffer and analyzed by SDS-PAGE.
Iminunofluorescence Microscopy. Cells cultured on gelatin-coated glass coverslips were fixed with 3.7% formaldehyde in PBS for 10 min at room temperature, permeabilized with 0.1% Triton X-100 for 5 min, and then washed with PBS three times. To block nonspecific binding, the cells were incubated with a solution of PBS containing 1% bovine serum albumin for 30 min and then incubated with primary antibody at appropriate dilutions for 1 h. After incubation with primary antibody, cells were washed three times with PBS and incubated with fluorescence-conjugated secondary antibody (Molecular Probes). The coverslips were then fixed onto slides and imaged using a Zeiss fluorescence microscope.
Electron Microscopy. Samples were absorbed onto freshly glow-discharged, carbon-coated copper grids for 2 minutes and stained with 2% uranyl acetate. Grids were examined using a Zeiss electron microscopy at an accelerating voltage of 80 kV.

### Results

To understand the molecular basis of the action of migrastatin analogs, the protein target of migrastatin analogs was identified. An unbiased approach towards the identification of the protein target employing a biotin-labeled macroketone was tested (see FIG. 4A). This biotin-labeled migrastatin analog was active in inhibiting the 4T1 breast tumor cell migration (Gaul et al. 2004). The biotin-labeled macroketone was used to set up an affinity column and the ˜55 kDa protein target was successfully purified (FIG. 4B) and identified as mouse fascin 1 by mass spectrometry.

Different but complementary approaches were used to verify fascin as the target. The first approach was in vitro studies on the interaction of migrastatin analogs with fascin. Fascin was purified as a GST-fusion protein from Escherichia coli (FIGS. 5A and 5B). Purified fascin, but not GST control, specifically interacted with biotin-conjugated macroketone (FIGS. 5A and 5B). Additionally, excess amount of non-biotinylated macroketone efficiently competed the binding between fascin and biotin-conjugated macroketone (FIG. 5C). Another migrastatin analog, macrolactam, also competed the binding of biotin-conjugated macroketone to fascin (data not shown). Collectively, these data demonstrate that fascin is a protein target of macroketone.

Three different approaches were used to investigate the effect of macroketone on fascin. First, the actin-bundling activity of purified recombinant fascin protein was investigated by the F-actin pelleting assay (Yamashiro-Matsumura et al. 1985). In this low-speed centrifugation assay, the pellets contain bundles of F-actin polymers. Purified fascin increased the amounts of F-actin bundles in the pellets (FIG. 6A). While macroketone alone had no effect on the formation of F-actin bundles, macroketone significantly decreased the fascin-induced bundling of F-actin polymers (FIG. 6A). Second, fluorescence microscopy was used to visualize the fascin-regulated F-actin filament bundles in the absence and presence of macroketone (FIG. 6B). Addition of fascin induced the formation of F-actin bundles, as revealed by the staining of F-actin filaments with Rhodamine-conjugated phalloidine (FIG. 6B). In contrast, in the presence of macroketone, formation of F-actin bundles was largely (>80%) inhibited (FIGS. 6B and 6C). Third, electron microscopy was used to examine the actin bundles (FIG. 6D). The EM examination revealed that macroketone decreased the thickness of the bundles (FIG. 6D). These thin F-actin bundles often had branches which were not observed in the absence of macroketone. For fascin to bundle F-actin polymers, fascin needs to bind to F-actin polymers. Thus, it is likely that macroketone inhibits the direct binding of fascin to F-actin. To confirm this, high-speed centrifugation method was used to pellet F-actin polymers (Yamashiro-Matsumura et al. 1985). Under these conditions, fascin alone was not precipitated and fascin could only be pulled-down by binding to F-actin polymers (Id.). While similar amounts of F-actin polymers were in the pellets in the absence and presence of macroketone (since the same amounts of F-actin polymers were added), significantly less fascin was pulled down by F-actin in the presence of macroketone (FIG. 6E). These data demonstrate that macroketone inhibits the actin-bundling activity of fascin.

### Example 6

### Essential Role for Fascin in Breast Tumor Cell Migration

### Methods

RNA interference. RNAi of fascin was performed in 4T1 mouse breast tumor and MDA-MB-231 human breast tumor cells using pSUPER vector (Oligoengine). The target sequences of the two pairs of mouse fascin were GGTGGGCAAAGATGAGCTC (SEQ ID NO:63) and GTGGAGCGTGCACATCGCC (SEQ ID NO:64). The target sequences of the two pairs of human fascin were GGTGGGCAAGGACGAGCTC (SEQ ID NO:65) and GCCTGAAGAAGAAGCAGAT (SEQ ID NO:66). One day before transfection, cells were plated in 0.5 ml of growth medium without antibiotics. At the time of transfection, the cells were 30-50% confluent. For each transfection sample, siRNA was prepared as follows:

1) Dilute the appropriate amount of siRNA in 50 μl of Opti-MEM I Reduced Serum Medium without serum (or other medium without serum). Mix gently.

2) Mix Lipofectamine 2000 gently before use, then dilute the appropriate amount in 50 μl of Opti-MEMI Medium (or other medium without serum). Mix gently and incubate for 5 minutes at room temperature. Note: Combine the diluted Lipofectamine 2000 with the diluted siRNA within 30 minutes. Longer incubation times may decrease activity. If D-MEM is used as a diluent for the Lipofectamine 2000, mix with the diluted siRNA within 5 minutes.

3) After the 5 minute incubation, combine the diluted siRNA with the diluted Lipofectamine 2000 (total volume is 100 μl). Mix gently and incubate for 20 minutes at room temperature to allow the siRNA:Lipofectamine 2000 complexes to form.

Add the 100 μl of siRNA:Lipofectamine 2000 complexes to each well. Mix gently by rocking the plate back and forth.

Cells were incubated at 37° C. in a CO₂ incubator for 24-72 hours until they were ready to assay for gene knockdown. It was generally not necessary to remove the complexes or change the medium; however, growth medium was replaced after 4-6 hours without loss of transfection activity.

The following additional cell lines were likewise tested with migrastatin analogs and fascin siRNA as described herein: human colon tumor Lovo-229 cells; human prostate tumor PC-3 cells; melanoma B16 cells; ovarian tumor cells; and lung tumor cells.

Boyden Chamber Cell Migration Assay. Cells (5×10⁴) suspended in starvation medium were added to the upper chamber of an insert (6.5 mm diameter, 8-micrometer pore size, Becton Dickenson), and the insert was placed in a 24-well dish containing starvation medium with or without 10% FBS (Yang et al. 2005, Shan et al. 2006). When used, inhibitors were added to both chambers. Migration assays were carried out for 4-6 hours and cells were fixed with 3.7% formaldehyde. Cells were stained with crystal violet staining solution, and cells on the upper side of the insert were removed with a cotton swab. Three randomly selected fields (10× objectives) on the lower side of the insert were photographed, and the migrated cells were counted. The migration was expressed as either the average number of migrated cells in a field or as percentage of migrated cells in positive control. Percentage was calculated with the formula P=100×(M−M_{nc})/M_{pc}, where P is the percentage of migrated cells, M is the number of migrated cells, M_{nc} is the number of migrated cells in negative controls, and M_{pc} is the number of migrated cells in positive controls.

### Results

The highly invasive tumor cell lines 4T1 mouse mammary tumor cells and MDA-MB-231 human breast tumor cells were used to test the effect of decreasing fascin protein levels in tumor cells. Two different siRNAs against mouse fascin-1 and one control siRNA were used to treat 4T1 cells and cells stably expressing these siRNAs were selected. While fascin siRNAs knocked down the fascin protein levels, the control siRNA did not (FIG. 7A). Fascin siRNA-treated cells grew at comparable rates as control siRNA-treated cells and non-transfected 4T1 cells in full growth medium (data not shown), suggesting fascin is not required for breast tumor cell proliferation in vitro. This is consistent with previous observations that migrastatin analogs had no obvious effect on tumor cell proliferation and primary tumor growth in mouse models (Shan et al. 2005). Boyden chamber cell migration assays showed that fascin siRNA treatments, but not treatment with the control siRNA, decreased the serum-induced migration of 4T1 cells (FIG. 7B). This inhibitory effect of fascin siRNA could be rescued by transfection of human fascin cDNA (there are two nucleotide changes without amino acid changes in this specific region) (FIGS. 7C and 7D). Similarly, fascin siRNA treatments down-regulated the fascin protein level and decreased the migration of MDA-MB-231 cells (FIGS. 7E and 7F). Fascin siRNA treatment did not affect the proliferation of MDA-MB-231 cells (data not shown). In addition to these loss-of-function analyses, gain-of-function experiments were also performed. Comparing to metastatic MDA-MB-231 human breast tumor cells, MCF-10A normal mammary gland epithelial cells expressed less amount of fascin proteins (FIG. 7G). Overexpression of fascin in MCF-10A cells increased the serum-induced migration of these cells (FIG. 7H). Together, these data demonstrate that fascin plays a critical role in the migration of breast tumor cells.

We have solved the X-ray crystal structure of the complex of fascin and macroketone (see Example 9). Based on the structure of the complex, His474 in human fascin is essential for the macroketone binding, but not for actin-bundling. Furthermore, His 474 is not conserved in Drosophila fascin, and Drosophila fascin could rescue the migration defect in 4T1 cells treated with fascin siRNAs with no sensitivity to macroketone (data not shown). As shown in FIG. 7I, while expression of human fascin in fascin siRNA-treated mouse 4T1 cells rescued the migration, this rescue was sensitive to macroketone. In contrast, mutations of His474 to either Lys (Drosophila fascin has a Lys in the corresponding position) or Ala in human fascin rescued the migration of 4T1 cells treated with fascin siRNAs (FIG. 7I). These rescues were not inhibited by macroketone. Additionally, rescue experiments of fascin-siRNA-treated 4T1 cells with villin, another actin-bundling protein, were performed. From Drosophila genetic studies, villin partially rescued the phenotypes of fascin mutations during Drosophila oogenesis (Cant et al. 1996). Villin did not bind macroketone in vitro, and over-expression of villin in fascin-siRNA treated 4T1 cells partially rescued the migration which was insensitive to macroketone (data not shown). These results further confirm that fascin is the protein target for macroketone in its inhibition of tumor cell migration.

### Example 7

### Inhibition of Fascin Blocks Breast Tumor Metastasis in Mouse Models

### Methods

Breast Tumor Metastasis in Mice. All animal work was performed in compliance with the Institutional Animal Care and Use Committee of the Weill Medical College. Spontaneous 4T1 mouse breast tumor metastasis assay was done as described previously (Shan et al. 2005). NOD-SCID immunodeficient mice were used for experimental lung metastasis experiments. MDA-MB-231 human breast tumor cells expressing the TGL reporter were trypsinized and washed with PBS. This artificial TGL reporter gene encodes a triple fusion protein with herpes simplex virus 1 thymidine kinase fused to the N-terminus of enhanced GFP and firefly luciferase fused to the C-terminus of GFP (Ponomarev et al. 2004). Subsequently 1×10⁶ cells in 0.2 ml PBS were injected into the lateral tail vein. Luciferase-based, noninvasive bioluminescent imaging and analysis were performed with an IVIS Imaging System (Xenogen).
Cell Invasion Assay. Cells (1×10⁵) suspended in starvation medium were added to the upper chamber of a Matrigel-coated insert (6.5 mm diameter, 8-μm pore size, Becton Dickenson), and the insert was placed in a 24-well dish containing medium with or without serum. When used, inhibitors were added to both chambers. Invasion assays were carried out for 16 hours and cells were fixed with 3.7% formaldehyde. Cells were stained with crystal violet staining solution, and cells on the upper side of the insert were removed with a cotton swab. Three randomly selected fields (10×objectives) on the lower side of the insert were photographed, and the cells on the lower surface of the insert were counted.

### Results

The role of fascin in tumor metastasis was tested in animal models. The spontaneous metastasis model (with 4T1 tumor cells) and the experimental metastasis model (with MDA-MB-231 tumor cells) were used. First, it was examined whether suppression of fascin inhibits tumor invasion through a 3D matrix. As shown in FIG. 8A, expression of two fascin siRNAs in 4T1 cells dramatically reduced the 4T1 tumor cell invasion. Similarly, suppression of fascin by siRNAs in human MDA-MB-231 breast tumor cells inhibited cell invasion (data not shown). Second, the spontaneous metastasis model with 4T1 tumor cells was used to investigate the role of fascin in tumor metastasis (FIG. 8 B-D). 4T1 cells were injected into mouse mammary glands. Primary tumors from both fascin siRNA-treated cells and control siRNA-treated cells developed at similar rates (FIG. 8B) and had similar weights four weeks later (FIG. 8C), confirming that suppression of fascin did not affect proliferation of 4T1 cells in vivo. Four weeks after transplantation of 4T1 tumor cells, mice were sacrificed and examined for tumor metastasis to the lung (FIG. 8D). While mice injected with control siRNA-treated cells showed large numbers of metastasized 4T1 cells in the lung, fascin siRNA-treated cells failed to metastasize to the lung (FIG. 8D).

Third, the experimental metastasis model with MDA-MB-231 human tumor cells in immunodeficient mice was used to investigate the role of fascin in tumor metastasis and the effect of macroketone on the metastasis of human tumors in mice (FIG. 8 E-H). MDA-MB-231 cells were retrovirally infected with a triple-fusion protein reporter construct encoding herpes simplex virus thymidine kinase 1, green fluorescent protein (GFP) and firefly luciferase (TGL) (Minn et al. 2005). GFP-positive cells were enriched by fluorescence-activated cell sorting. These cells were injected into the tail vein of immunodeficient mice [NOD-SCID mice]. The metastasis of tumor cells to the lung was monitored by non-invasive bioluminescence imaging (Minn et al. 2005).

Most of these tumor cells became trapped in the capillaries of the lungs shortly after injection (due to size restrictions imposed by mouse capillaries, human tumor cells are rarely able to pass from the arterial to the venous system (or vice versa) by way of the lung (Minn et al. 2005) (FIG. 8E, Day 0). A substantial attenuation of bioluminescence signal was observed within the first few days, indicating that cells that failed to metastasize were not able to survive (FIGS. 8E and 8F). Progressively increasing signals after two weeks in mice with control shRNA-treated (stably expressing siRNA) tumor cells indicated that cells had succeeded in metastasizing and proliferating (FIGS. 8E and 8F). Strikingly, the presence of fascin shRNA treated cells (stably expressing siRNAs) in the lung was much less than control shRNA-treated cells (FIGS. 8E and 8F). Therefore, fascin siRNA treatments significantly inhibited breast tumor metastasis.

To further confirm the inhibition of tumor metastasis, histological analyses of the lung tissues from xenografted mice were performed (FIG. 8G). Lung tissues from xenografted mice were isolated and sectioned. Hematoxylin and eosin (H&E) staining showed normal structure of the lungs from mice injected with fascin siRNA-treated MDA-MB-231 tumor cells (FIG. 8G). In contrast, lung tissues from mice injected with control shRNA-treated tumor cells were heavily infiltrated by metastasized human breast tumor cells (FIG. 8G). The identity of tumor cells in the lung tissue was confirmed by GFP fluorescence since the injected MDA-MB-231 tumor cells were labeled with GFP (FIG. 8G). These results demonstrate that fascin is critical for human tumor metastasis in a mouse model.

Furthermore, it was demonstrated here that macroketone could effectively block the metastasis of human breast tumors in an animal model. The NOD-SCID mice were injected with MDA-MB-231 tumor cells with the triple-fusion protein reporter. Macroketone (10 mg/kg) or the control saline (PBS) was administered (via I.P.) on every other day for seven weeks. The effect of macroketone on the metastasis of human breast tumor cells to the lung was monitored using LivingImage software (Xenogen) by measurement of photon flux. As shown in FIG. 8H, macroketone reduced the metastasis of MDA-MB-231 cells by >80%. Together, the data demonstrate an essential role for fascin in breast tumor metastasis, and the feasibility of using the inhibitors of fascin (such as macroketone and siRNAs) as therapeutic agents for treating metastatic breast tumors.

### Example 8

### Elevated Expression of Fascin in Human Breast Cancer Patients

### Methods

Microarray Gene Expression Analysis. Gene expression data for fascin was extracted from each tumor sample and mean-centered across all samples for each. Tissues from primary breast cancers were obtained from therapeutic procedures performed as part of routine clinical management at Memorial Sloan-Kettering Cancer Center. All research procedures using human tissue were approved by the MSKCC institutional review board (Doane et al. 2006). Tissues were snap-frozen in liquid nitrogen and stored at −80° C. Each sample was examined histologically using hemotoxylin- and eosin-stained cryostat sections. Regions were manually dissected from the frozen block to provide a consistent tumor cell content of more than 70% in tissues used for analysis. Total RNA was extracted from frozen tissue by homogenization in guanidinium isothiocyanate-based buffer (Trizol; Invitrogen, Carlsbad, Calif.), purified using RNAeasy (Qiagen, Valencia, Calif.) and examined for quality using denaturing agarose gel. Complementary DNA was synthesized from RNA using a T7-promoter-tagged oligo-dT primer. RNA target was synthesized from cDNA by in vitro transcription, and labeled with biotinylated nucleotides (Enzo Biochem, Farmingdale, N.Y.). Gene expression analysis was performed using HG-U133A and U133B oligonucleotide microarrays according to the manufacturer's instructions (Affymetrix, Santa Clara, Calif.). To identify the differential gene expression, two different measures were used:fold change (ratio) between the normalized means of each group of samples and a Student's t-test.

### Results

Fascin expression levels in tumor samples from human breast cancer patients were examined. A microarray gene expression data set from 137 breast cancer samples and 16 normal breast samples was analyzed. Breast tumor samples showed elevated fascin expressions comparing to normal samples (FIG. 9A). Moreover, a significant high level of fascin transcripts in the Estrogen Receptor (ER)-negative group of patients (FIG. 9B) and Progesterone Receptor (PR)-negative group of patients (FIG. 9C) was observed. Immunohistology staining with anti-fascin antibody confirmed that fascin protein was up regulated in ER-negative tumors (FIG. 9D), while ER-positive tumor cells were negative for fascin staining (note that endothelia of vessels are fascin positive). These data reveal that fascin transcripts and protein levels are significantly elevated in aggressive ER-negative breast tumors.

Fascin mRNA expression levels in the Rosetta microarray data set of 295 breast cancer patients was also analyzed (van de Vijver et al. 2002, van 't Veer et al. 2002). Similarly, levels of fascin transcripts were significantly higher in ER-negative (FIG. 9G) and PR-negative (FIG. 9H) tumors. The Rosetta data set contains detailed clinical follow-up information of breast cancer patients. Thus, the clinical and pathological associations of fascin expression in breast cancer patients was evaluated. Kaplan-Meier analyses showed that higher fascin expression was associated with lower overall survival (FIG. 9E) and lower metastasis-free survival (FIG. 9F). These data highlight the correlation between higher fascin expression and metastasis and death in human breast cancer patients.

### Example 9

### Structural Basis for the Inhibition of Fascin Function and Tumor Metastasis by Migrastatin Analogs

The X-ray crystal structures of fascin in the absence and in the presence of a migrastatin analog were determined. Migrastatin analogs bind to fascin in a groove that has been biochemically and genetically defined as the surface for actin binding. These structural data provide a molecular basis for the inhibition of fascin by migrastatin analogs.

### Methods

Human fascin-1 expression and purification. Recombinant human fascin-1 was expressed as GST-fusion protein in E. coli. Typically, a 1 liter 2YT medium with antibiotic was inoculated with 3 ml overnight BL21 culture transformed with pGEX4T-Fascinl plasmid and grown at 37° C. until OD₆₀₀ reached ˜0.8. The culture was then transferred to 22° C. and 0.1 mM IPTG was added for induction. After overnight induction, the bacteria were harvested by centrifugation at 5,000 rpm for 10 min. The bacteria pellet was snap frozen with liquid nitrogen and suspended in 30 ml 1×PBS supplemented with 0.2 mM PMSF, 1 mM DTT, 1% Triton X-100 and 1 mM EDTA. After sonication, the suspension was centrifuged at 15,000 rpm for 60 min to remove the cell debris. The supernatant was then incubated with 4 ml glutathione beads (Sigma) at 4° C. for 2 hours. After extensive wash with PBS, the beads were resuspended in 10 ml thrombin cleavage buffer (20 mM Tris, pH8.0, 150 mM NaCl, 2 mM CaCl₂, 1 mM DTT). Human Fascin-1 was released from the beads by incubating with 40-100 units of thrombin overnight at 4° C. After centrifugation, 0.2 mM PMSF was added to the supernatant to inactivate the remnant thrombin activity. The fascin protein was further purified with a Superdex 200 gel filtration column and concentrated with Centricon to about 80 mg/ml. The typical yield from a 1 liter culture is about 40 mg.
Crystallization and structure determination. Concentrated fascin stock was diluted with fascin buffer (20 mM Tris, pH8.0, 40 mM KBr, 0.5 mM EDTA, 1 mM DTT) to 15 mg/ml. For the growth of fascin-macroketone complex, the protein was incubated with 2 mM macroketone at room temperature for 1 hour. The crystal drops were set up by hanging drop diffusion at 20° C. in reservoir solution that contained 100 mM Hepes, pH8.0, 16% PEG4000, 1% isopropanol. Crystals were harvest in cryo-solution (100 mM Hepes, pH8.0, 16% PEG400, 15% glycerol) and snap frozen in liquid nitrogen. X-ray diffraction data were collected from frozen crystals at National Synchrotron Light Source beamline X6a at Brookhaven National Laboratory. The atomic models of fascin and fascin-macroketone complex were initially obtained by molecular replacement with ldfc model using Phaser. The structures were manually adjusted with Coot and refined with CNS and Refmac5 with R_{free} sets containing 5% of the reflections. Two fascin molecules were found in each asymmetric unit.
Actin bundling assay. The actin bundling assay was performed as described in Example 4 above.

### Results

Overall Structure and Topology of Human Fascin-1. The X-ray crystal structure of native human fascin-1 as well as fascin-1 in complex with a migrastatin analog, the macroketone core, was determined (FIG. 10A, B and C). Both crystals belong to C2 space group. The native fascin structure and the structure of fascin-macroketone complex was determined at 2.1 Å and 2.7 Å, respectively (FIG. 10 A, B and C). The overall structure of fascin exhibits four β-trefoil folds, with β-trefoil 1 and 2 forming a dumbbell-shaped domain, and β-trefoil 3 and 4 forming another (FIG. 10A). The two dumbbells are inter-connected by a loop between β-trefoil 2 and 3. The two dumbbell domains are arranged in a way that trefoil 2 directly contacts trefoil 3 and 4, while trefoil 4 directly contacts trefoil 1 and 2 (FIG. 10A). Overall, the two dumbbells create a horseshoe appearance.
Migrastatin Analog Binding Pocket. The overall domain arrangement of fascin-macroketone complex is very similar to that of the native fascin, with two dumbbells forming the two arms of a horseshoe (FIG. 10C). A 3σ F_{obs}-F_{calc} electron density peak was observed on the surface of β-trefoil 4 (FIG. 11A). The macrolide ring of macroketone fits well with the extra density. The bound macroketone molecule sits at the surface of trefoil 4, on the side facing the cleft between trefoil 4 and trefoil 1 (FIG. 10C). Macroketone is held in place by interacting with the side chains of His392, Glu391, Ala488, Lys471, and His474 as well as the alpha carbon of Asp473 (FIG. 11A-D). The six residues form a U-shape curvature, holding macroketone like holding a ring with thumb and index finger (FIGS. 11A and 11B). On the top of the two “fingers” are the two histidines, His392 and His474, which have major contributions to the fascin-macroketone interaction. The NE2 nitrogen of His392 is 3.01 Å away from the ketone oxygen of macroketone molecule, while the ND1 nitrogen of His474 is 2.57 Å away from the hydroxyl oxygen. His392 and His474 contribute to the binding of macroketone by forming hydrogen bonds with macroketone (FIG. 11B). The interaction between fascin and macroketone is further stabilized by the van der Waals force between the macrolide ring carbon and residue Glu391, Ala488, Lys471 and Asp473 (FIG. 11B).

Although the overall structure of fascin-macroketone complex is similar to the native fascin, with a root mean square deviation (RMSD) of 0.3 Å for all the alpha carbon atoms (FIG. 11C), several residues at the “thumb-and-index-finger” binding site for macroketone move as a result of “induced-fit” mechanism (FIG. 11D). While the alpha Cα of His474 moves about 2 Å away from the macroketone, its imidazole group is rotated by 180° about its Cα-Cβ bond toward the molecule. Consequently, the ND1 nitrogen of His474 moves 2.3 Å closer to form hydrogen bond with the hydroxyl group of macroketone. Meanwhile, the imidazole group of His392, which forms hydrogen bond with the ketone group of macroketone, is pushed 1 Å away. The carboxyl group of Asp473 also rotates 90° about its Cβ-Cγ bound as a consequence of the inhibitor-fascin interaction.

Actin binding sites. Fascin functions as a monomer to bundle actin filaments, and it has been proposed that fascin has two actin-binding sites for this bundling activity (Ono et al. 1997). The crystal structure shown herein provides a structural explanation for this (FIG. 12A and 12B, orange and cyan labeled residues). Both the N- and C-termini are located in the same cleft (FIGS. 12A and 12B). Furthermore, a stretch of residues from 29 to 42 at the N-terminal, which has similarity to an actin binding site of MARCKS (myristoylated alanine-rich C-kinase substrate), is also facing the trefoil 1-4 cleft (FIG. 12C, orange labeled residues in the original). Moreover, the actin bundling activity of fascin is negatively regulated by a protein kinase C phosphorylation site (Ser39) within the N-terminal region (FIG. 12D, the red labeled residue in the original). Together, these data suggest that this cleft represents one of the two actin-binding sites.

Genetic analysis of the Drosophila fascin homolog, singed, yielded two point mutations of fascin which are critical for its actin bundling activity (Cant et al. 1996). One mutation is Gly393 (GlY409 in Drosophila) to Glu that reduced the actin-bundling activity of fascin (FIG. 12D, the red label residue). This Gly393 locates in the above-mentioned actin binding site. On the other hand, another singed mutant is Ser274 (Ser289 in Drosophila) to Asn that almost eliminated the actin-bundling activity of fascin (FIG. 12E). This Ser274 locates on the opposite side of fascin (FIG. 12E). This surface may represent the second actin-binding site.

Biochemical and structural studies of the interaction of F-actin filaments and fimbrin, another actin bundling protein revealed two actin-binding sites. These two actin-binding sites on fimbrin are located in similar surfaces as the two potential actin-binding sites of fascin. Even though fimbrin consists of entirely α-helical structures and fascin with all β-sheets, they have similar overall structural arrangements.

Macroketone binds to one of the actin binding sites on fascin. The structure of the fascin-macroketone complex immediately suggested a possible mechanism by which macroketone inhibits the actin bundling activity of fascin. The macroketone binding site is one of the actin binding sites on fascin (FIG. 13A). Therefore, although not being bound by any specific theory, it appears that macroketone binding interferes with the binding of actin filament binding to fascin (FIG. 13B).

Five residues involved in macroketone binding were mutated and the actin bundling activity of those fascin mutants was examined (FIG. 14). Based on the actin bundling assays, His392, Lys471 and Ala488 are critical for actin bundling, while Glu391 and His474 are not (FIGS. 14A and 14B). Furthermore, the sensitivity of the actin bundling activity of Glu391 and His474 to macroketone was examined (mutants His392, Lys471 and Ala488 were not tested due to their defective actin bundling activity). As shown in FIG. 14C, mutation of His474 to Ala rendered fascin to resistant to macroketone treatment. Therefore, His474 is essential for macroketone binding. Taken together, this data demonstrates that several fascin residues involved in macroketone binding also contribute to actin binding. Hence, the macroketone binding site is one of the actin binding sites.

TABLE 2
Atomic Coordinates for Fascin

| REMARK | coordinates from water picking | |
|---|---|---|
| REMARK | 59 waters picked at level greater than 3.0 | |
| REMARK | in (1 m\|Fo\| − 1 D\|Fc\|)e\hat{ }(i phi_calc) cross-val. sigmaa map | |
| REMARK | peak selection criteria: hbond | |
| REMARK | peaks closer than 2.6 A or further than 4.0 A were deleted | |
| REMARK | but peaks 2.0 A from oxygen or nitrogen were kept | |
| REMARK | peaks further than 3.2 A from a oxygen or nitrogen were deleted | |
| REMARK | map resolution: 30-2.7 A | |
| REMARK | starting | r= 0.2707 free_r= 0.2898 |
| REMARK | final | r= 0.2666 free_r= 0.2874 |

| REMARK | sg=C2 a=160.358 b=70.407 c=112.398 alpha=90 beta=131.890 gamma=90 | | |
|---|---|---|---|
| REMARK | parameter file 1 : CNS_TOPPAR:protein_rep.param | | |
| REMARK | parameter file 2 : CNS_TOPPAR:dna-rna_rep.param | | |
| REMARK | parameter file 3 : CNS_TOPPAR:water_rep.param | | |
| REMARK | parameter file 4 : CNS_TOPPAR:ion.param | | |
| REMARK | parameter file 5 : ../xyz.param | | |
| REMARK | molecular structure file: ../gen_xyz.mtf | | |
| REMARK | input coordinates: ../gen_xyz.pdb | | |
| REMARK | anomalous f′ f″ library: CNS_XRAYLIB:anom_cu.lib | | |
| REMARK | reflection file= ../../070919.cv | | |
| REMARK | ncs= none | | |
| REMARK | B-correction resolution: 6.0-2.7 | | |
| REMARK | initial B-factor correction applied to fobs: | | |
| REMARK | B11= −5.627 | B22= 16.015 | B33= −10.388 |
| REMARK | B12= 0.000 | B13= −17.572 | B23= 0.000 |

| REMARK | B-factor correction applied to coordinate array B: 0.103 | | |
|---|---|---|---|
| REMARK | bulk solvent: density level = 0.262554 e/A\hat{ }3, B-factor= 50.3504 A\hat{ }2 | | |
| REMARK | reflections with \|Fobs\|/sigma_F < 0.0 rejected | | |
| REMARK | reflections with \|Fobs\| > 10000 * rms(Fobs) rejected | | |
| REMARK | theoretical total number of refl. in resol. range: | 25837 | (100.0%) |
| REMARK | number unobserved reflections (no entry or \|F\|=0): | 1298 | (5.0%) |
| REMARK | number reflections rejected: | 0 | (0.0%) |
| REMARK | total number of reflections used: | 24539 | (95.0%) |
| REMARK | number of reflections in working set: | 23345 | (90.4%) |
| REMARK | number of reflections in test set: | 1194 | (4.6%) |

| | |
|---|---|
| CRYST1 | 160.358 70.407 112.398 90.00 131.89 90.00 C 2 |
| REMARK | FILENAME=“wat_keton.pdb” |
| REMARK | VERSION: 1.1 |

| Atom | | Amino Acid | | | | | | | | |
|---|---|---|---|---|---|---|---|---|---|---|
| 1 | C | GLY | A | 1005 | −31.443 | −4.644 | 38.038 | 1.00 | 63.56 | A |
| 2 | O | GLY | A | 1005 | −32.038 | −3.530 | 38.056 | 1.00 | 62.44 | A |
| 3 | N | GLY | A | 1005 | −29.088 | −5.801 | 37.808 | 1.00 | 56.65 | A |
| 4 | CA | GLY | A | 1005 | −29.928 | −4.728 | 38.385 | 1.00 | 60.77 | A |
| 5 | N | THR | A | 1006 | −32.092 | −5.782 | 37.702 | 1.00 | 65.25 | A |
| 6 | CA | THR | A | 1006 | −33.445 | −5.702 | 37.031 | 1.00 | 65.48 | A |
| 7 | CB | THR | A | 1006 | −34.439 | −5.824 | 38.145 | 1.00 | 65.56 | A |
| 8 | OG1 | THR | A | 1006 | −33.942 | −4.955 | 39.157 | 1.00 | 68.68 | A |
| 9 | CG2 | THR | A | 1006 | −34.358 | −7.219 | 38.631 | 1.00 | 64.18 | A |
| 10 | C | THR | A | 1006 | −33.760 | −4.410 | 36.107 | 1.00 | 64.13 | A |
| 11 | O | THR | A | 1006 | −34.699 | −3.708 | 36.296 | 1.00 | 62.77 | A |
| 12 | N | ALA | A | 1007 | −32.966 | −4.151 | 35.080 | 1.00 | 64.41 | A |
| 13 | CA | ALA | A | 1007 | −32.720 | −2.714 | 34.527 | 1.00 | 63.53 | A |
| 14 | CB | ALA | A | 1007 | −33.362 | −1.508 | 35.354 | 1.00 | 60.50 | A |
| 15 | C | ALA | A | 1007 | −31.234 | −2.448 | 34.187 | 1.00 | 61.16 | A |
| 16 | O | ALA | A | 1007 | −30.964 | −1.986 | 33.050 | 1.00 | 62.88 | A |
| 17 | N | GLU | A | 1008 | −30.320 | −2.906 | 35.035 | 1.00 | 56.50 | A |
| 18 | CA | GLU | A | 1008 | −28.925 | −2.599 | 35.003 | 1.00 | 57.05 | A |
| 19 | CB | GLU | A | 1008 | −28.186 | −3.748 | 34.434 | 1.00 | 57.04 | A |
| 20 | CG | GLU | A | 1008 | −28.631 | −4.104 | 32.945 | 1.00 | 65.47 | A |
| 21 | CD | GLU | A | 1008 | −27.582 | −4.861 | 32.133 | 1.00 | 61.58 | A |
| 22 | OE1 | GLU | A | 1008 | −27.882 | −5.875 | 31.423 | 1.00 | 57.53 | A |
| 23 | OE2 | GLU | A | 1008 | −26.439 | −4.360 | 32.233 | 1.00 | 68.27 | A |
| 24 | C | GLU | A | 1008 | −28.470 | −1.263 | 34.370 | 1.00 | 57.18 | A |
| 25 | O | GLU | A | 1008 | −27.819 | −1.227 | 33.302 | 1.00 | 59.63 | A |
| 26 | N | ALA | A | 1009 | −28.775 | −0.132 | 34.995 | 1.00 | 55.46 | A |
| 27 | CA | ALA | A | 1009 | −28.384 | 1.140 | 34.402 | 1.00 | 54.55 | A |
| 28 | CB | ALA | A | 1009 | −29.029 | 2.248 | 35.181 | 1.00 | 54.39 | A |
| 29 | C | ALA | A | 1009 | −26.855 | 1.395 | 34.317 | 1.00 | 54.99 | A |
| 30 | O | ALA | A | 1009 | −26.047 | 0.785 | 35.022 | 1.00 | 56.25 | A |
| 31 | N | VAL | A | 1010 | −26.443 | 2.365 | 33.526 | 1.00 | 52.26 | A |
| 32 | CA | VAL | A | 1010 | −25.062 | 2.642 | 33.504 | 1.00 | 48.76 | A |
| 33 | CB | VAL | A | 1010 | −24.714 | 3.185 | 32.068 | 1.00 | 47.46 | A |
| 34 | CG1 | VAL | A | 1010 | −25.535 | 4.409 | 31.877 | 1.00 | 51.64 | A |
| 35 | CG2 | VAL | A | 1010 | −23.307 | 3.638 | 31.932 | 1.00 | 44.47 | A |
| 36 | C | VAL | A | 1010 | −24.654 | 3.566 | 34.725 | 1.00 | 49.13 | A |
| 37 | O | VAL | A | 1010 | −25.368 | 4.455 | 35.313 | 1.00 | 47.71 | A |
| 38 | N | GLN | A | 1011 | −23.423 | 3.380 | 35.086 | 1.00 | 48.76 | A |
| 39 | CA | GLN | A | 1011 | −22.969 | 4.026 | 36.226 | 1.00 | 48.93 | A |
| 40 | CB | GLN | A | 1011 | −22.387 | 3.046 | 37.343 | 1.00 | 47.97 | A |
| 41 | CG | GLN | A | 1011 | −21.624 | 3.899 | 38.409 | 1.00 | 50.69 | A |
| 42 | CD | GLN | A | 1011 | −20.686 | 3.136 | 39.306 | 1.00 | 52.22 | A |
| 43 | OE1 | GLN | A | 1011 | −19.537 | 3.027 | 38.983 | 1.00 | 52.51 | A |
| 44 | NE2 | GLN | A | 1011 | −21.182 | 2.581 | 40.455 | 1.00 | 56.23 | A |
| 45 | C | GLN | A | 1011 | −21.974 | 4.891 | 35.592 | 1.00 | 45.39 | A |
| 46 | O | GLN | A | 1011 | −20.977 | 4.392 | 35.155 | 1.00 | 45.78 | A |
| 47 | N | ILE | A | 1012 | −22.259 | 6.202 | 35.656 | 1.00 | 45.34 | A |
| 48 | CA | ILE | A | 1012 | −21.454 | 7.310 | 35.208 | 1.00 | 42.98 | A |
| 49 | CB | ILE | A | 1012 | −22.198 | 8.623 | 35.253 | 1.00 | 43.06 | A |
| 50 | CG2 | ILE | A | 1012 | −21.435 | 9.629 | 34.467 | 1.00 | 39.58 | A |
| 51 | CG1 | ILE | A | 1012 | −23.537 | 8.635 | 34.563 | 1.00 | 44.21 | A |
| 52 | CD1 | ILE | A | 1012 | −23.898 | 7.513 | 33.615 | 1.00 | 47.82 | A |
| 53 | C | ILE | A | 1012 | −20.283 | 7.466 | 36.121 | 1.00 | 44.47 | A |
| 54 | O | ILE | A | 1012 | −20.437 | 7.549 | 37.331 | 1.00 | 43.06 | A |
| 55 | N | GLN | A | 1013 | −19.144 | 7.534 | 35.470 | 1.00 | 45.23 | A |
| 56 | CA | GLN | A | 1013 | −17.860 | 7.721 | 35.934 | 1.00 | 48.94 | A |
| 57 | CB | GLN | A | 1013 | −17.030 | 6.407 | 35.828 | 1.00 | 51.51 | A |
| 58 | CG | GLN | A | 1013 | −17.399 | 5.553 | 37.087 | 1.00 | 51.83 | A |
| 59 | CD | GLN | A | 1013 | −16.392 | 4.558 | 37.594 | 1.00 | 50.69 | A |
| 60 | OE1 | GLN | A | 1013 | −15.797 | 3.768 | 36.837 | 1.00 | 57.64 | A |
| 61 | NE2 | GLN | A | 1013 | −16.242 | 4.525 | 38.929 | 1.00 | 53.80 | A |
| 62 | C | GLN | A | 1013 | −17.261 | 8.769 | 35.059 | 1.00 | 51.14 | A |
| 63 | O | GLN | A | 1013 | −17.304 | 8.672 | 33.836 | 1.00 | 54.16 | A |
| 64 | N | PHE | A | 1014 | −16.661 | 9.798 | 35.666 | 1.00 | 54.55 | A |
| 65 | CA | PHE | A | 1014 | −15.954 | 10.855 | 34.881 | 1.00 | 52.56 | A |
| 66 | CB | PHE | A | 1014 | −16.895 | 11.801 | 34.338 | 1.00 | 50.01 | A |
| 67 | CG | PHE | A | 1014 | −17.703 | 12.476 | 35.300 | 1.00 | 48.05 | A |
| 68 | CD1 | PHE | A | 1014 | −17.238 | 13.529 | 35.997 | 1.00 | 46.54 | A |
| 69 | CD2 | PHE | A | 1014 | −18.984 | 12.161 | 35.457 | 1.00 | 47.73 | A |
| 70 | CE1 | PHE | A | 1014 | −18.106 | 14.281 | 36.835 | 1.00 | 44.30 | A |
| 71 | CE2 | PHE | A | 1014 | −19.838 | 12.871 | 36.354 | 1.00 | 47.96 | A |
| 72 | CZ | PHE | A | 1014 | −19.379 | 13.929 | 37.031 | 1.00 | 46.03 | A |
| 73 | C | PHE | A | 1014 | −14.757 | 11.540 | 35.451 | 1.00 | 53.11 | A |
| 74 | O | PHE | A | 1014 | −14.515 | 11.602 | 36.605 | 1.00 | 54.55 | A |
| 75 | N | GLY | A | 1015 | −13.918 | 11.979 | 34.587 | 1.00 | 53.94 | A |
| 76 | CA | GLY | A | 1015 | −12.948 | 13.012 | 35.018 | 1.00 | 54.68 | A |
| 77 | C | GLY | A | 1015 | −13.478 | 14.427 | 34.870 | 1.00 | 54.65 | A |
| 78 | O | GLY | A | 1015 | −14.241 | 14.800 | 33.871 | 1.00 | 55.71 | A |
| 79 | N | LEU | A | 1016 | −13.113 | 15.260 | 35.832 | 1.00 | 54.35 | A |
| 80 | CA | LEU | A | 1016 | −13.655 | 16.708 | 35.822 | 1.00 | 55.52 | A |
| 81 | CB | LEU | A | 1016 | −14.531 | 16.970 | 36.986 | 1.00 | 54.00 | A |
| 82 | CG | LEU | A | 1016 | −15.879 | 17.697 | 36.981 | 1.00 | 54.40 | A |
| 83 | CD1 | LEU | A | 1016 | −16.686 | 17.585 | 35.673 | 1.00 | 59.29 | A |
| 84 | CD2 | LEU | A | 1016 | −16.625 | 17.246 | 38.252 | 1.00 | 49.57 | A |
| 85 | C | LEU | A | 1016 | −12.480 | 17.679 | 35.737 | 1.00 | 55.53 | A |
| 86 | O | LEU | A | 1016 | −11.480 | 17.411 | 36.361 | 1.00 | 54.23 | A |
| 87 | N | ILE | A | 1017 | −12.554 | 18.622 | 34.780 | 1.00 | 57.79 | A |
| 88 | CA | ILE | A | 1017 | −11.364 | 19.364 | 34.146 | 1.00 | 60.86 | A |
| 89 | CB | ILE | A | 1017 | −11.268 | 19.112 | 32.589 | 1.00 | 62.78 | A |
| 90 | CG2 | ILE | A | 1017 | −10.082 | 19.879 | 31.923 | 1.00 | 59.65 | A |
| 91 | CG1 | ILE | A | 1017 | −10.985 | 17.633 | 32.267 | 1.00 | 63.94 | A |
| 92 | CD1 | ILE | A | 1017 | −11.025 | 17.347 | 30.631 | 1.00 | 63.88 | A |
| 93 | C | ILE | A | 1017 | −11.385 | 20.908 | 34.261 | 1.00 | 58.77 | A |
| 94 | O | ILE | A | 1017 | −12.198 | 21.546 | 33.673 | 1.00 | 55.86 | A |
| 95 | N | ASN | A | 1018 | −10.472 | 21.444 | 35.058 | 1.00 | 59.04 | A |
| 96 | CA | ASN | A | 1018 | −10.460 | 22.895 | 35.499 | 1.00 | 58.78 | A |
| 97 | CB | ASN | A | 1018 | −10.271 | 23.020 | 37.084 | 1.00 | 57.34 | A |
| 98 | CG | ASN | A | 1018 | −8.905 | 23.615 | 37.519 | 1.00 | 53.04 | A |
| 99 | OD1 | ASN | A | 1018 | −7.796 | 23.066 | 37.248 | 1.00 | 44.38 | A |
| 100 | ND2 | ASN | A | 1018 | −8.989 | 24.776 | 38.180 | 1.00 | 50.40 | A |
| 101 | C | ASN | A | 1018 | −9.431 | 23.838 | 34.892 | 1.00 | 59.06 | A |
| 102 | O | ASN | A | 1018 | −8.240 | 23.409 | 34.404 | 1.00 | 58.09 | A |
| 103 | N | CYS | A | 1019 | −9.816 | 25.098 | 35.153 | 1.00 | 57.40 | A |
| 104 | CA | CYS | A | 1019 | −8.958 | 26.299 | 34.922 | 1.00 | 57.47 | A |
| 105 | CB | CYS | A | 1019 | −8.570 | 26.929 | 36.237 | 1.00 | 57.44 | A |
| 106 | SG | CYS | A | 1019 | −9.436 | 28.406 | 36.579 | 1.00 | 64.67 | A |
| 107 | C | CYS | A | 1019 | −7.740 | 25.864 | 34.224 | 1.00 | 53.98 | A |
| 108 | O | CYS | A | 1019 | −7.850 | 25.354 | 33.207 | 1.00 | 55.20 | A |
| 109 | N | GLY | A | 1020 | −6.593 | 26.003 | 34.798 | 1.00 | 52.78 | A |
| 110 | CA | GLY | A | 1020 | −5.395 | 25.267 | 34.338 | 1.00 | 54.49 | A |
| 111 | C | GLY | A | 1020 | −5.401 | 23.751 | 33.866 | 1.00 | 52.79 | A |
| 112 | O | GLY | A | 1020 | −4.440 | 23.061 | 34.077 | 1.00 | 49.42 | A |
| 113 | N | ASN | A | 1021 | −6.421 | 23.274 | 33.161 | 1.00 | 53.56 | A |
| 114 | CA | ASN | A | 1021 | −6.248 | 21.945 | 32.548 | 1.00 | 57.24 | A |
| 115 | CB | ASN | A | 1021 | −5.281 | 21.939 | 31.283 | 1.00 | 57.08 | A |
| 116 | CG | ASN | A | 1021 | −5.988 | 21.633 | 29.826 | 1.00 | 51.13 | A |
| 117 | OD1 | ASN | A | 1021 | −5.309 | 21.269 | 28.861 | 1.00 | 51.04 | A |
| 118 | ND2 | ASN | A | 1021 | −7.249 | 21.809 | 29.706 | 1.00 | 42.97 | A |
| 119 | C | ASN | A | 1021 | −5.561 | 21.096 | 33.734 | 1.00 | 59.54 | A |
| 120 | O | ASN | A | 1021 | −4.461 | 20.385 | 33.460 | 1.00 | 59.25 | A |
| 121 | N | LYS | A | 1022 | −6.123 | 21.237 | 34.999 | 1.00 | 56.29 | A |
| 122 | CA | LYS | A | 1022 | −5.871 | 20.258 | 36.031 | 1.00 | 52.35 | A |
| 123 | CB | LYS | A | 1022 | −5.330 | 20.966 | 37.254 | 1.00 | 50.78 | A |
| 124 | CG | LYS | A | 1022 | −3.985 | 21.344 | 37.117 | 1.00 | 43.26 | A |
| 125 | CD | LYS | A | 1022 | −3.055 | 20.215 | 36.580 | 1.00 | 40.80 | A |
| 126 | CE | LYS | A | 1022 | −1.568 | 20.479 | 36.850 | 1.00 | 31.19 | A |
| 127 | NZ | LYS | A | 1022 | −0.689 | 20.298 | 35.669 | 1.00 | 44.88 | A |
| 128 | C | LYS | A | 1022 | −7.182 | 19.503 | 36.385 | 1.00 | 54.00 | A |
| 129 | O | LYS | A | 1022 | −8.242 | 20.122 | 36.575 | 1.00 | 56.17 | A |
| 130 | N | TYR | A | 1023 | −7.149 | 18.176 | 36.447 | 1.00 | 53.84 | A |
| 131 | CA | TYR | A | 1023 | −8.370 | 17.376 | 36.798 | 1.00 | 53.31 | A |
| 132 | CB | TYR | A | 1023 | −8.031 | 15.899 | 36.552 | 1.00 | 52.89 | A |
| 133 | CG | TYR | A | 1023 | −8.144 | 15.497 | 35.188 | 1.00 | 50.27 | A |
| 134 | CD1 | TYR | A | 1023 | −7.085 | 15.373 | 34.422 | 1.00 | 56.94 | A |
| 135 | CE1 | TYR | A | 1023 | −7.203 | 15.011 | 33.082 | 1.00 | 58.64 | A |
| 136 | CD2 | TYR | A | 1023 | −9.369 | 15.271 | 34.626 | 1.00 | 51.76 | A |
| 137 | CE2 | TYR | A | 1023 | −9.512 | 14.955 | 33.319 | 1.00 | 48.09 | A |
| 138 | CZ | TYR | A | 1023 | −8.425 | 14.786 | 32.552 | 1.00 | 55.01 | A |
| 139 | OH | TYR | A | 1023 | −8.562 | 14.364 | 31.215 | 1.00 | 55.49 | A |
| 140 | C | TYR | A | 1023 | −8.718 | 17.482 | 38.298 | 1.00 | 52.99 | A |
| 141 | O | TYR | A | 1023 | −7.860 | 17.564 | 39.098 | 1.00 | 54.53 | A |
| 142 | N | LEU | A | 1024 | −9.938 | 17.272 | 38.650 | 1.00 | 52.04 | A |
| 143 | CA | LEU | A | 1024 | −10.379 | 17.200 | 40.023 | 1.00 | 53.31 | A |
| 144 | CB | LEU | A | 1024 | −11.920 | 17.015 | 39.977 | 1.00 | 52.76 | A |
| 145 | CG | LEU | A | 1024 | −12.669 | 17.757 | 41.065 | 1.00 | 53.91 | A |
| 146 | CD1 | LEU | A | 1024 | −14.036 | 17.164 | 41.086 | 1.00 | 43.01 | A |
| 147 | CD2 | LEU | A | 1024 | −11.902 | 17.825 | 42.609 | 1.00 | 51.84 | A |
| 148 | C | LEU | A | 1024 | −9.753 | 16.080 | 40.891 | 1.00 | 52.85 | A |
| 149 | O | LEU | A | 1024 | −9.661 | 14.948 | 40.447 | 1.00 | 56.13 | A |
| 150 | N | THR | A | 1025 | −9.332 | 16.319 | 42.125 | 1.00 | 52.61 | A |
| 151 | CA | THR | A | 1025 | −8.397 | 15.249 | 42.730 | 1.00 | 52.97 | A |
| 152 | CB | THR | A | 1025 | −7.044 | 15.367 | 42.179 | 1.00 | 50.65 | A |
| 153 | OG1 | THR | A | 1025 | −7.278 | 15.190 | 40.852 | 1.00 | 52.08 | A |
| 154 | CG2 | THR | A | 1025 | −6.170 | 14.310 | 42.559 | 1.00 | 47.81 | A |
| 155 | C | THR | A | 1025 | −8.395 | 14.877 | 44.248 | 1.00 | 54.00 | A |
| 156 | O | THR | A | 1025 | −8.002 | 15.670 | 45.098 | 1.00 | 52.19 | A |
| 157 | N | ALA | A | 1026 | −8.894 | 13.675 | 44.552 | 1.00 | 54.51 | A |
| 158 | CA | ALA | A | 1026 | −8.824 | 13.212 | 45.909 | 1.00 | 55.31 | A |
| 159 | CB | ALA | A | 1026 | −10.028 | 12.637 | 46.306 | 1.00 | 53.96 | A |
| 160 | C | ALA | A | 1026 | −7.650 | 12.277 | 46.062 | 1.00 | 55.88 | A |
| 161 | O | ALA | A | 1026 | −7.548 | 11.276 | 45.445 | 1.00 | 57.50 | A |
| 162 | N | GLU | A | 1027 | −6.743 | 12.621 | 46.927 | 1.00 | 55.61 | A |
| 163 | CA | GLU | A | 1027 | −5.492 | 12.001 | 46.893 | 1.00 | 55.01 | A |
| 164 | CB | GLU | A | 1027 | −4.478 | 13.044 | 47.260 | 1.00 | 54.55 | A |
| 165 | CG | GLU | A | 1027 | −4.521 | 14.391 | 46.447 | 1.00 | 55.17 | A |
| 166 | CD | GLU | A | 1027 | −3.520 | 14.307 | 45.359 | 1.00 | 55.86 | A |
| 167 | OE1 | GLU | A | 1027 | −3.154 | 15.269 | 44.596 | 1.00 | 55.85 | A |
| 168 | OE2 | GLU | A | 1027 | −3.034 | 13.193 | 45.377 | 1.00 | 55.71 | A |
| 169 | C | GLU | A | 1027 | −5.625 | 11.049 | 48.023 | 1.00 | 57.42 | A |
| 170 | O | GLU | A | 1027 | −6.747 | 10.854 | 48.608 | 1.00 | 55.47 | A |
| 171 | N | ALA | A | 1028 | −4.468 | 10.460 | 48.395 | 1.00 | 57.69 | A |
| 172 | CA | ALA | A | 1028 | −4.549 | 9.518 | 49.458 | 1.00 | 57.44 | A |
| 173 | CB | ALA | A | 1028 | −3.610 | 8.317 | 49.175 | 1.00 | 58.00 | A |
| 174 | C | ALA | A | 1028 | −4.284 | 10.175 | 50.861 | 1.00 | 58.03 | A |
| 175 | O | ALA | A | 1028 | −4.102 | 9.419 | 51.865 | 1.00 | 59.82 | A |
| 176 | N | PHE | A | 1029 | −4.209 | 11.517 | 50.881 | 1.00 | 57.48 | A |
| 177 | CA | PHE | A | 1029 | −3.890 | 12.433 | 52.034 | 1.00 | 57.08 | A |
| 178 | CB | PHE | A | 1029 | −3.433 | 13.850 | 51.543 | 1.00 | 59.26 | A |
| 179 | CG | PHE | A | 1029 | −2.189 | 13.877 | 50.866 | 1.00 | 57.88 | A |
| 180 | CD1 | PHE | A | 1029 | −2.169 | 14.031 | 49.448 | 1.00 | 59.87 | A |
| 181 | CD2 | PHE | A | 1029 | −1.026 | 13.752 | 51.610 | 1.00 | 57.84 | A |
| 182 | CE1 | PHE | A | 1029 | −0.978 | 13.930 | 48.672 | 1.00 | 53.85 | A |
| 183 | CE2 | PHE | A | 1029 | 0.201 | 13.720 | 50.953 | 1.00 | 67.06 | A |
| 184 | CZ | PHE | A | 1029 | 0.243 | 13.854 | 49.433 | 1.00 | 65.71 | A |
| 185 | C | PHE | A | 1029 | −5.161 | 12.861 | 52.684 | 1.00 | 55.47 | A |
| 186 | O | PHE | A | 1029 | −6.147 | 13.143 | 51.957 | 1.00 | 56.57 | A |
| 187 | N | GLY | A | 1030 | −5.137 | 12.979 | 53.978 | 1.00 | 50.11 | A |
| 188 | CA | GLY | A | 1030 | −5.988 | 13.905 | 54.585 | 1.00 | 50.10 | A |
| 189 | C | GLY | A | 1030 | −7.388 | 14.051 | 54.169 | 1.00 | 50.62 | A |
| 190 | O | GLY | A | 1030 | −8.121 | 14.830 | 54.733 | 1.00 | 51.04 | A |
| 191 | N | PHE | A | 1031 | −7.790 | 13.224 | 53.220 | 1.00 | 50.67 | A |
| 192 | CA | PHE | A | 1031 | −8.910 | 13.497 | 52.358 | 1.00 | 50.15 | A |
| 193 | CB | PHE | A | 1031 | −10.238 | 13.520 | 53.055 | 1.00 | 49.29 | A |
| 194 | CG | PHE | A | 1031 | −10.476 | 12.283 | 53.875 | 1.00 | 54.80 | A |
| 195 | CD1 | PHE | A | 1031 | −11.344 | 12.296 | 54.938 | 1.00 | 55.12 | A |
| 196 | CD2 | PHE | A | 1031 | −9.774 | 11.051 | 53.572 | 1.00 | 57.41 | A |
| 197 | CE1 | PHE | A | 1031 | −11.425 | 11.192 | 55.768 | 1.00 | 60.16 | A |
| 198 | CE2 | PHE | A | 1031 | −9.905 | 9.964 | 54.376 | 1.00 | 58.47 | A |
| 199 | CZ | PHE | A | 1031 | −10.728 | 10.024 | 55.459 | 1.00 | 57.52 | A |
| 200 | C | PHE | A | 1031 | −8.730 | 14.718 | 51.615 | 1.00 | 51.59 | A |
| 201 | O | PHE | A | 1031 | −9.488 | 15.609 | 51.795 | 1.00 | 51.95 | A |
| 202 | N | LYS | A | 1032 | −7.758 | 14.794 | 50.728 | 1.00 | 53.43 | A |
| 203 | CA | LYS | A | 1032 | −7.706 | 16.026 | 49.994 | 1.00 | 56.72 | A |
| 204 | CB | LYS | A | 1032 | −6.341 | 16.682 | 50.093 | 1.00 | 55.47 | A |
| 205 | CG | LYS | A | 1032 | −6.294 | 17.746 | 51.123 | 1.00 | 54.23 | A |
| 206 | CD | LYS | A | 1032 | −5.107 | 17.362 | 52.207 | 1.00 | 55.22 | A |
| 207 | CE | LYS | A | 1032 | −4.473 | 18.539 | 52.956 | 1.00 | 47.58 | A |
| 208 | NZ | LYS | A | 1032 | −5.599 | 19.425 | 53.489 | 1.00 | 43.63 | A |
| 209 | C | LYS | A | 1032 | −8.199 | 15.947 | 48.527 | 1.00 | 59.09 | A |
| 210 | O | LYS | A | 1032 | −7.974 | 14.908 | 47.835 | 1.00 | 60.43 | A |
| 211 | N | VAL | A | 1033 | −8.839 | 17.067 | 48.110 | 1.00 | 59.33 | A |
| 212 | CA | VAL | A | 1033 | −9.520 | 17.275 | 46.835 | 1.00 | 58.64 | A |
| 213 | CB | VAL | A | 1033 | −11.057 | 17.328 | 47.124 | 1.00 | 58.02 | A |
| 214 | CG1 | VAL | A | 1033 | −11.312 | 18.381 | 48.239 | 1.00 | 59.34 | A |
| 215 | CG2 | VAL | A | 1033 | −11.755 | 17.752 | 45.949 | 1.00 | 54.93 | A |
| 216 | C | VAL | A | 1033 | −8.981 | 18.561 | 46.065 | 1.00 | 58.41 | A |
| 217 | O | VAL | A | 1033 | −9.529 | 19.667 | 46.071 | 1.00 | 57.02 | A |
| 218 | N | ASN | A | 1034 | −7.908 | 18.373 | 45.332 | 1.00 | 59.46 | A |
| 219 | CA | ASN | A | 1034 | −7.249 | 19.515 | 44.700 | 1.00 | 59.67 | A |
| 220 | CB | ASN | A | 1034 | −5.731 | 19.495 | 44.859 | 1.00 | 56.12 | A |
| 221 | CG | ASN | A | 1034 | −5.135 | 18.242 | 44.369 | 1.00 | 56.34 | A |
| 222 | OD1 | ASN | A | 1034 | −5.229 | 17.937 | 43.158 | 1.00 | 61.05 | A |
| 223 | ND2 | ASN | A | 1034 | −4.470 | 17.443 | 45.305 | 1.00 | 51.67 | A |
| 224 | C | ASN | A | 1034 | −7.613 | 19.513 | 43.228 | 1.00 | 60.16 | A |
| 225 | O | ASN | A | 1034 | −8.550 | 18.798 | 42.809 | 1.00 | 60.76 | A |
| 226 | N | ALA | A | 1035 | −6.895 | 20.379 | 42.499 | 1.00 | 58.69 | A |
| 227 | CA | ALA | A | 1035 | −6.887 | 20.397 | 41.074 | 1.00 | 57.21 | A |
| 228 | CB | ALA | A | 1035 | −7.342 | 21.722 | 40.556 | 1.00 | 56.17 | A |
| 229 | C | ALA | A | 1035 | −5.457 | 20.331 | 40.868 | 1.00 | 57.36 | A |
| 230 | O | ALA | A | 1035 | −4.954 | 21.267 | 40.397 | 1.00 | 58.76 | A |
| 231 | N | SER | A | 1036 | −4.763 | 19.252 | 41.258 | 1.00 | 58.33 | A |
| 232 | CA | SER | A | 1036 | −3.423 | 19.142 | 40.880 | 1.00 | 56.02 | A |
| 233 | CB | SER | A | 1036 | −2.504 | 19.404 | 42.026 | 1.00 | 56.56 | A |
| 234 | OG | SER | A | 1036 | −2.294 | 20.862 | 42.070 | 1.00 | 52.28 | A |
| 235 | C | SER | A | 1036 | −2.908 | 18.337 | 39.670 | 1.00 | 57.13 | A |
| 236 | O | SER | A | 1036 | −1.727 | 18.556 | 39.257 | 1.00 | 57.60 | A |
| 237 | N | ALA | A | 1037 | −3.874 | 17.749 | 38.941 | 1.00 | 56.75 | A |
| 238 | CA | ALA | A | 1037 | −3.868 | 16.429 | 38.298 | 1.00 | 56.13 | A |
| 239 | CB | ALA | A | 1037 | −5.246 | 15.897 | 38.585 | 1.00 | 55.02 | A |
| 240 | C | ALA | A | 1037 | −3.754 | 16.565 | 36.781 | 1.00 | 57.24 | A |
| 241 | O | ALA | A | 1037 | −4.554 | 17.355 | 36.119 | 1.00 | 54.07 | A |
| 242 | N | SER | A | 1038 | −2.805 | 15.849 | 36.167 | 1.00 | 58.31 | A |
| 243 | CA | SER | A | 1038 | −2.584 | 16.230 | 34.703 | 1.00 | 59.47 | A |
| 244 | CB | SER | A | 1038 | −1.209 | 16.909 | 34.522 | 1.00 | 58.39 | A |
| 245 | OG | SER | A | 1038 | −0.144 | 15.988 | 34.401 | 1.00 | 55.29 | A |
| 246 | C | SER | A | 1038 | −2.705 | 15.058 | 33.783 | 1.00 | 59.94 | A |
| 247 | O | SER | A | 1038 | −1.792 | 14.792 | 33.072 | 1.00 | 60.52 | A |
| 248 | N | SER | A | 1039 | −3.800 | 14.298 | 33.916 | 1.00 | 60.43 | A |
| 249 | CA | SER | A | 1039 | −3.994 | 12.917 | 33.372 | 1.00 | 57.59 | A |
| 250 | CB | SER | A | 1039 | −2.801 | 12.043 | 33.700 | 1.00 | 56.68 | A |
| 251 | OG | SER | A | 1039 | −3.031 | 10.742 | 33.239 | 1.00 | 56.29 | A |
| 252 | C | SER | A | 1039 | −5.228 | 12.391 | 34.033 | 1.00 | 56.58 | A |
| 253 | O | SER | A | 1039 | −5.500 | 12.792 | 35.122 | 1.00 | 57.19 | A |
| 254 | N | LEU | A | 1040 | −6.063 | 11.635 | 33.336 | 1.00 | 57.07 | A |
| 255 | CA | LEU | A | 1040 | −7.133 | 10.817 | 34.010 | 1.00 | 56.28 | A |
| 256 | CB | LEU | A | 1040 | −8.294 | 10.499 | 33.051 | 1.00 | 55.51 | A |
| 257 | CG | LEU | A | 1040 | −9.806 | 10.756 | 33.251 | 1.00 | 52.43 | A |
| 258 | CD1 | LEU | A | 1040 | −10.564 | 9.742 | 32.516 | 1.00 | 49.92 | A |
| 259 | CD2 | LEU | A | 1040 | −10.291 | 10.778 | 34.711 | 1.00 | 50.34 | A |
| 260 | C | LEU | A | 1040 | −6.554 | 9.469 | 34.459 | 1.00 | 56.62 | A |
| 261 | O | LEU | A | 1040 | −6.335 | 8.672 | 33.614 | 1.00 | 55.18 | A |
| 262 | N | LYS | A | 1041 | −6.255 | 9.289 | 35.793 | 1.00 | 59.43 | A |
| 263 | CA | LYS | A | 1041 | −5.888 | 7.984 | 36.569 | 1.00 | 55.50 | A |
| 264 | CB | LYS | A | 1041 | −4.469 | 7.956 | 37.081 | 1.00 | 54.44 | A |
| 265 | CG | LYS | A | 1041 | −3.441 | 8.411 | 36.085 | 1.00 | 52.16 | A |
| 266 | CD | LYS | A | 1041 | −3.846 | 7.799 | 34.618 | 1.00 | 58.24 | A |
| 267 | CE | LYS | A | 1041 | −3.131 | 6.336 | 34.256 | 1.00 | 52.00 | A |
| 268 | NZ | LYS | A | 1041 | −3.760 | 5.654 | 33.060 | 1.00 | 49.99 | A |
| 269 | C | LYS | A | 1041 | −6.757 | 7.938 | 37.754 | 1.00 | 55.56 | A |
| 270 | O | LYS | A | 1041 | −7.710 | 8.676 | 37.842 | 1.00 | 57.31 | A |
| 271 | N | LYS | A | 1042 | −6.484 | 7.028 | 38.677 | 1.00 | 55.81 | A |
| 272 | CA | LYS | A | 1042 | −7.457 | 6.648 | 39.701 | 1.00 | 53.94 | A |
| 273 | CB | LYS | A | 1042 | −6.816 | 5.776 | 40.706 | 1.00 | 53.20 | A |
| 274 | CG | LYS | A | 1042 | −6.934 | 4.328 | 40.409 | 1.00 | 57.97 | A |
| 275 | CD | LYS | A | 1042 | −5.563 | 3.807 | 39.917 | 1.00 | 59.08 | A |
| 276 | CE | LYS | A | 1042 | −4.698 | 3.164 | 41.081 | 1.00 | 52.12 | A |
| 277 | NZ | LYS | A | 1042 | −3.345 | 2.820 | 40.751 | 1.00 | 37.82 | A |
| 278 | C | LYS | A | 1042 | −8.047 | 7.785 | 40.483 | 1.00 | 54.82 | A |
| 279 | O | LYS | A | 1042 | −9.254 | 7.756 | 40.786 | 1.00 | 57.56 | A |
| 280 | N | LYS | A | 1043 | −7.251 | 8.793 | 40.910 | 1.00 | 54.21 | A |
| 281 | CA | LYS | A | 1043 | −7.796 | 9.757 | 41.964 | 1.00 | 50.42 | A |
| 282 | CB | LYS | A | 1043 | −6.653 | 10.501 | 42.658 | 1.00 | 48.64 | A |
| 283 | CG | LYS | A | 1043 | −5.936 | 9.825 | 43.796 | 1.00 | 47.06 | A |
| 284 | CD | LYS | A | 1043 | −4.634 | 9.320 | 43.398 | 1.00 | 37.10 | A |
| 285 | CE | LYS | A | 1043 | −3.627 | 9.756 | 44.259 | 1.00 | 39.10 | A |
| 286 | NZ | LYS | A | 1043 | −2.363 | 10.523 | 43.584 | 1.00 | 39.03 | A |
| 287 | C | LYS | A | 1043 | −8.594 | 10.783 | 41.170 | 1.00 | 50.70 | A |
| 288 | O | LYS | A | 1043 | −9.137 | 11.768 | 41.750 | 1.00 | 50.11 | A |
| 289 | N | GLN | A | 1044 | −8.539 | 10.647 | 39.808 | 1.00 | 50.83 | A |
| 290 | CA | GLN | A | 1044 | −9.185 | 11.587 | 38.850 | 1.00 | 50.83 | A |
| 291 | CB | GLN | A | 1044 | −8.375 | 11.693 | 37.616 | 1.00 | 52.80 | A |
| 292 | CG | GLN | A | 1044 | −7.048 | 12.599 | 37.687 | 1.00 | 56.90 | A |
| 293 | CD | GLN | A | 1044 | −6.165 | 12.507 | 38.974 | 1.00 | 55.33 | A |
| 294 | OE1 | GLN | A | 1044 | −5.494 | 11.531 | 39.255 | 1.00 | 61.26 | A |
| 295 | NE2 | GLN | A | 1044 | −6.076 | 13.566 | 39.623 | 1.00 | 60.28 | A |
| 296 | C | GLN | A | 1044 | −10.616 | 11.184 | 38.493 | 1.00 | 50.18 | A |
| 297 | O | GLN | A | 1044 | −11.497 | 12.050 | 38.383 | 1.00 | 51.43 | A |
| 298 | N | ILE | A | 1045 | −10.869 | 9.879 | 38.463 | 1.00 | 49.98 | A |
| 299 | CA | ILE | A | 1045 | −12.221 | 9.247 | 38.411 | 1.00 | 50.18 | A |
| 300 | CB | ILE | A | 1045 | −12.232 | 7.758 | 37.903 | 1.00 | 48.74 | A |
| 301 | CG2 | ILE | A | 1045 | −11.251 | 7.502 | 36.648 | 1.00 | 49.78 | A |
| 302 | CG1 | ILE | A | 1045 | −11.726 | 6.852 | 38.879 | 1.00 | 52.23 | A |
| 303 | CD1 | ILE | A | 1045 | −12.465 | 5.526 | 38.804 | 1.00 | 56.67 | A |
| 304 | C | ILE | A | 1045 | −13.155 | 9.494 | 39.607 | 1.00 | 49.28 | A |
| 305 | O | ILE | A | 1045 | −12.859 | 9.204 | 40.717 | 1.00 | 51.66 | A |
| 306 | N | TRP | A | 1046 | −14.275 | 10.116 | 39.318 | 1.00 | 49.66 | A |
| 307 | CA | TRP | A | 1046 | −15.277 | 10.535 | 40.216 | 1.00 | 50.01 | A |
| 308 | CB | TRP | A | 1046 | −15.555 | 12.077 | 40.014 | 1.00 | 49.08 | A |
| 309 | CG | TRP | A | 1046 | −14.479 | 12.821 | 40.716 | 1.00 | 47.72 | A |
| 310 | CD2 | TRP | A | 1046 | −14.377 | 13.050 | 42.124 | 1.00 | 42.59 | A |
| 311 | CE2 | TRP | A | 1046 | −13.068 | 13.513 | 42.385 | 1.00 | 53.93 | A |
| 312 | CE3 | TRP | A | 1046 | −15.250 | 12.921 | 43.169 | 1.00 | 46.79 | A |
| 313 | CD1 | TRP | A | 1046 | −13.229 | 13.115 | 40.198 | 1.00 | 49.26 | A |
| 314 | NE1 | TRP | A | 1046 | −12.368 | 13.530 | 41.182 | 1.00 | 47.35 | A |
| 315 | CZ2 | TRP | A | 1046 | −12.638 | 13.917 | 43.726 | 1.00 | 49.17 | A |
| 316 | CZ3 | TRP | A | 1046 | −14.853 | 13.346 | 44.499 | 1.00 | 51.34 | A |
| 317 | CH2 | TRP | A | 1046 | −13.581 | 13.864 | 44.740 | 1.00 | 48.93 | A |
| 318 | C | TRP | A | 1046 | −16.399 | 9.631 | 39.676 | 1.00 | 52.70 | A |
| 319 | O | TRP | A | 1046 | −16.531 | 9.581 | 38.427 | 1.00 | 54.08 | A |
| 320 | N | THR | A | 1047 | −17.122 | 8.887 | 40.560 | 1.00 | 52.16 | A |
| 321 | CA | THR | A | 1047 | −18.348 | 8.282 | 40.168 | 1.00 | 52.53 | A |
| 322 | CB | THR | A | 1047 | −18.350 | 6.810 | 40.436 | 1.00 | 53.10 | A |
| 323 | OG1 | THR | A | 1047 | −19.609 | 6.362 | 40.991 | 1.00 | 49.98 | A |
| 324 | CG2 | THR | A | 1047 | −17.193 | 6.440 | 41.295 | 1.00 | 55.96 | A |
| 325 | C | THR | A | 1047 | −19.668 | 9.016 | 40.509 | 1.00 | 54.13 | A |
| 326 | O | THR | A | 1047 | −19.798 | 9.737 | 41.505 | 1.00 | 51.28 | A |
| 327 | N | LEU | A | 1048 | −20.649 | 8.875 | 39.610 | 1.00 | 56.84 | A |
| 328 | CA | LEU | A | 1048 | −21.944 | 9.560 | 39.842 | 1.00 | 60.48 | A |
| 329 | CB | LEU | A | 1048 | −22.315 | 10.273 | 38.570 | 1.00 | 60.57 | A |
| 330 | CG | LEU | A | 1048 | −23.408 | 11.311 | 38.377 | 1.00 | 61.49 | A |
| 331 | CD1 | LEU | A | 1048 | −22.720 | 12.338 | 37.463 | 1.00 | 56.88 | A |
| 332 | CD2 | LEU | A | 1048 | −24.828 | 10.744 | 37.822 | 1.00 | 56.83 | A |
| 333 | C | LEU | A | 1048 | −22.988 | 8.554 | 40.373 | 1.00 | 61.68 | A |
| 334 | O | LEU | A | 1048 | −22.738 | 7.328 | 40.315 | 1.00 | 63.95 | A |
| 335 | N | GLU | A | 1049 | −24.093 | 9.030 | 40.942 | 1.00 | 63.28 | A |
| 336 | CA | GLU | A | 1049 | −24.963 | 8.193 | 41.909 | 1.00 | 65.68 | A |
| 337 | CB | GLU | A | 1049 | −25.196 | 9.064 | 43.135 | 1.00 | 63.34 | A |
| 338 | CG | GLU | A | 1049 | −25.022 | 8.332 | 44.443 | 1.00 | 64.20 | A |
| 339 | CD | GLU | A | 1049 | −23.793 | 7.344 | 44.495 | 1.00 | 67.57 | A |
| 340 | OE1 | GLU | A | 1049 | −23.579 | 6.460 | 45.426 | 1.00 | 56.24 | A |
| 341 | OE2 | GLU | A | 1049 | −23.030 | 7.472 | 43.530 | 1.00 | 72.03 | A |
| 342 | C | GLU | A | 1049 | −26.325 | 7.311 | 41.509 | 1.00 | 67.14 | A |
| 343 | O | GLU | A | 1049 | −27.069 | 7.559 | 40.511 | 1.00 | 67.34 | A |
| 344 | N | ASN | A | 1050 | −26.592 | 6.273 | 42.319 | 1.00 | 68.93 | A |
| 345 | CA | ASN | A | 1050 | −27.764 | 5.349 | 42.315 | 1.00 | 69.51 | A |
| 346 | CB | ASN | A | 1050 | −27.545 | 4.196 | 43.290 | 1.00 | 69.29 | A |
| 347 | CG | ASN | A | 1050 | −26.906 | 2.987 | 42.665 | 1.00 | 75.36 | A |
| 348 | OD1 | ASN | A | 1050 | −25.679 | 2.820 | 42.744 | 1.00 | 78.45 | A |
| 349 | ND2 | ASN | A | 1050 | −27.737 | 2.063 | 42.120 | 1.00 | 80.85 | A |
| 350 | C | ASN | A | 1050 | −29.084 | 5.937 | 42.842 | 1.00 | 71.36 | A |
| 351 | O | ASN | A | 1050 | −29.122 | 6.371 | 44.029 | 1.00 | 71.37 | A |
| 352 | N | PRO | A | 1051 | −30.191 | 5.738 | 42.037 | 1.00 | 71.73 | A |
| 353 | CD | PRO | A | 1051 | −30.298 | 4.529 | 41.192 | 1.00 | 71.02 | A |
| 354 | CA | PRO | A | 1051 | −31.405 | 6.475 | 41.929 | 1.00 | 71.56 | A |
| 355 | CB | PRO | A | 1051 | −32.435 | 5.363 | 42.077 | 1.00 | 73.05 | A |
| 356 | CG | PRO | A | 1051 | −31.563 | 3.946 | 41.674 | 1.00 | 71.00 | A |
| 357 | C | PRO | A | 1051 | −31.475 | 7.460 | 43.084 | 1.00 | 72.99 | A |
| 358 | O | PRO | A | 1051 | −31.784 | 7.039 | 44.230 | 1.00 | 73.02 | A |
| 359 | N | PRO | A | 1052 | −30.922 | 8.712 | 42.888 | 1.00 | 72.66 | A |
| 360 | CD | PRO | A | 1052 | −29.663 | 9.182 | 42.223 | 1.00 | 70.84 | A |
| 361 | CA | PRO | A | 1052 | −31.469 | 9.717 | 43.894 | 1.00 | 70.92 | A |
| 362 | CB | PRO | A | 1052 | −30.444 | 10.916 | 43.848 | 1.00 | 72.03 | A |
| 363 | CG | PRO | A | 1052 | −28.996 | 10.029 | 43.444 | 1.00 | 71.02 | A |
| 364 | C | PRO | A | 1052 | −32.960 | 9.936 | 43.809 | 1.00 | 68.27 | A |
| 365 | O | PRO | A | 1052 | −33.604 | 8.838 | 44.021 | 1.00 | 65.07 | A |
| 366 | N | SER | A | 1057 | −33.444 | 14.359 | 44.475 | 1.00 | 63.42 | A |
| 367 | CA | SER | A | 1057 | −34.318 | 14.468 | 43.314 | 1.00 | 64.32 | A |
| 368 | CB | SER | A | 1057 | −35.322 | 15.553 | 43.606 | 1.00 | 66.51 | A |
| 369 | OG | SER | A | 1057 | −34.585 | 16.798 | 43.673 | 1.00 | 70.24 | A |
| 370 | C | SER | A | 1057 | −33.653 | 14.753 | 41.924 | 1.00 | 64.44 | A |
| 371 | O | SER | A | 1057 | −33.476 | 13.817 | 41.215 | 1.00 | 67.06 | A |
| 372 | N | ALA | A | 1058 | −33.325 | 16.027 | 41.553 | 1.00 | 63.17 | A |
| 373 | CA | ALA | A | 1058 | −32.898 | 16.527 | 40.192 | 1.00 | 60.13 | A |
| 374 | CB | ALA | A | 1058 | −33.256 | 17.905 | 40.074 | 1.00 | 56.63 | A |
| 375 | C | ALA | A | 1058 | −31.425 | 16.428 | 40.102 | 1.00 | 59.93 | A |
| 376 | O | ALA | A | 1058 | −30.757 | 16.399 | 39.054 | 1.00 | 57.61 | A |
| 377 | N | ALA | A | 1059 | −30.907 | 16.309 | 41.315 | 1.00 | 61.50 | A |
| 378 | CA | ALA | A | 1059 | −29.494 | 16.502 | 41.612 | 1.00 | 59.67 | A |
| 379 | CB | ALA | A | 1059 | −29.431 | 17.377 | 42.760 | 1.00 | 61.08 | A |
| 380 | C | ALA | A | 1059 | −28.707 | 15.237 | 41.840 | 1.00 | 58.62 | A |
| 381 | O | ALA | A | 1059 | −29.257 | 14.070 | 41.943 | 1.00 | 61.34 | A |
| 382 | N | VAL | A | 1060 | −27.407 | 15.424 | 41.911 | 1.00 | 57.35 | A |
| 383 | CA | VAL | A | 1060 | −26.487 | 14.300 | 42.007 | 1.00 | 55.63 | A |
| 384 | CB | VAL | A | 1060 | −25.693 | 14.247 | 40.654 | 1.00 | 58.16 | A |
| 385 | CG1 | VAL | A | 1060 | −26.718 | 14.045 | 39.336 | 1.00 | 53.15 | A |
| 386 | CG2 | VAL | A | 1060 | −24.675 | 15.523 | 40.640 | 1.00 | 50.87 | A |
| 387 | C | VAL | A | 1060 | −25.368 | 14.499 | 43.032 | 1.00 | 55.20 | A |
| 388 | O | VAL | A | 1060 | −24.975 | 15.630 | 43.350 | 1.00 | 52.12 | A |
| 389 | N | CYS | A | 1061 | −24.800 | 13.346 | 43.423 | 1.00 | 55.62 | A |
| 390 | CA | CYS | A | 1061 | −23.539 | 13.251 | 44.177 | 1.00 | 55.25 | A |
| 391 | CB | CYS | A | 1061 | −23.823 | 12.788 | 45.656 | 1.00 | 55.10 | A |
| 392 | SG | CYS | A | 1061 | −25.650 | 12.699 | 46.018 | 1.00 | 57.66 | A |
| 393 | C | CYS | A | 1061 | −22.367 | 12.419 | 43.515 | 1.00 | 55.44 | A |
| 394 | O | CYS | A | 1061 | −22.547 | 11.540 | 42.679 | 1.00 | 58.20 | A |
| 395 | N | LEU | A | 1062 | −21.153 | 12.719 | 43.919 | 1.00 | 53.61 | A |
| 396 | CA | LEU | A | 1062 | −20.035 | 12.273 | 43.265 | 1.00 | 51.95 | A |
| 397 | CB | LEU | A | 1062 | −19.330 | 13.501 | 42.588 | 1.00 | 54.30 | A |
| 398 | CG | LEU | A | 1062 | −19.619 | 14.436 | 41.370 | 1.00 | 52.80 | A |
| 399 | CD1 | LEU | A | 1062 | −21.022 | 14.893 | 41.275 | 1.00 | 50.19 | A |
| 400 | CD2 | LEU | A | 1062 | −18.649 | 15.672 | 41.318 | 1.00 | 49.44 | A |
| 401 | C | LEU | A | 1062 | −19.125 | 11.785 | 44.420 | 1.00 | 51.88 | A |
| 402 | O | LEU | A | 1062 | −18.788 | 12.623 | 45.413 | 1.00 | 50.85 | A |
| 403 | N | ARG | A | 1063 | −18.658 | 10.529 | 44.250 | 1.00 | 46.31 | A |
| 404 | CA | ARG | A | 1063 | −17.838 | 10.002 | 45.186 | 1.00 | 45.80 | A |
| 405 | CB | ARG | A | 1063 | −18.500 | 8.949 | 46.044 | 1.00 | 49.53 | A |
| 406 | CG | ARG | A | 1063 | −19.272 | 7.714 | 45.395 | 1.00 | 52.99 | A |
| 407 | CD | ARG | A | 1063 | −18.686 | 6.485 | 46.142 | 1.00 | 53.69 | A |
| 408 | NE | ARG | A | 1063 | −19.659 | 5.621 | 46.660 | 1.00 | 52.00 | A |
| 409 | CZ | ARG | A | 1063 | −19.291 | 4.458 | 47.074 | 1.00 | 52.14 | A |
| 410 | NH1 | ARG | A | 1063 | −18.017 | 4.283 | 47.085 | 1.00 | 46.67 | A |
| 411 | NH2 | ARG | A | 1063 | −20.185 | 3.572 | 47.583 | 1.00 | 56.13 | A |
| 412 | C | ARG | A | 1063 | −16.555 | 9.646 | 44.611 | 1.00 | 44.08 | A |
| 413 | O | ARG | A | 1063 | −16.525 | 9.592 | 43.355 | 1.00 | 43.08 | A |
| 414 | N | SER | A | 1064 | −15.503 | 9.597 | 45.479 | 1.00 | 40.24 | A |
| 415 | CA | SER | A | 1064 | −14.189 | 9.347 | 45.082 | 1.00 | 41.91 | A |
| 416 | CB | SER | A | 1064 | −13.244 | 9.946 | 46.046 | 1.00 | 41.70 | A |
| 417 | OG | SER | A | 1064 | −13.882 | 10.026 | 47.256 | 1.00 | 51.98 | A |
| 418 | C | SER | A | 1064 | −13.969 | 7.881 | 45.087 | 1.00 | 43.92 | A |
| 419 | O | SER | A | 1064 | −14.863 | 7.149 | 45.494 | 1.00 | 43.09 | A |
| 420 | N | HIS | A | 1065 | −12.826 | 7.382 | 44.602 | 1.00 | 47.72 | A |
| 421 | CA | HIS | A | 1065 | −12.549 | 5.926 | 44.774 | 1.00 | 50.85 | A |
| 422 | CB | HIS | A | 1065 | −11.220 | 5.487 | 44.284 | 1.00 | 48.86 | A |
| 423 | CG | HIS | A | 1065 | −11.241 | 4.052 | 43.765 | 1.00 | 54.02 | A |
| 424 | CD2 | HIS | A | 1065 | −10.256 | 3.298 | 43.207 | 1.00 | 59.11 | A |
| 425 | ND1 | HIS | A | 1065 | −12.376 | 3.284 | 43.683 | 1.00 | 54.21 | A |
| 426 | CE1 | HIS | A | 1065 | −12.045 | 2.082 | 43.246 | 1.00 | 63.10 | A |
| 427 | NE2 | HIS | A | 1065 | −10.758 | 2.066 | 42.944 | 1.00 | 52.59 | A |
| 428 | C | HIS | A | 1065 | −12.518 | 5.383 | 46.197 | 1.00 | 55.25 | A |
| 429 | O | HIS | A | 1065 | −12.760 | 4.106 | 46.437 | 1.00 | 57.42 | A |
| 430 | N | LEU | A | 1066 | −12.115 | 6.286 | 47.139 | 1.00 | 55.58 | A |
| 431 | CA | LEU | A | 1066 | −11.741 | 5.859 | 48.466 | 1.00 | 52.27 | A |
| 432 | CB | LEU | A | 1066 | −11.022 | 6.953 | 49.171 | 1.00 | 52.78 | A |
| 433 | CG | LEU | A | 1066 | −9.495 | 7.143 | 48.817 | 1.00 | 53.96 | A |
| 434 | CD1 | LEU | A | 1066 | −9.026 | 8.757 | 48.914 | 1.00 | 51.52 | A |
| 435 | CD2 | LEU | A | 1066 | −8.504 | 6.115 | 49.444 | 1.00 | 40.36 | A |
| 436 | C | LEU | A | 1066 | −13.112 | 5.666 | 48.956 | 1.00 | 52.84 | A |
| 437 | O | LEU | A | 1066 | −13.353 | 4.828 | 49.917 | 1.00 | 55.58 | A |
| 438 | N | GLY | A | 1067 | −14.038 | 6.252 | 48.196 | 1.00 | 48.20 | A |
| 439 | CA | GLY | A | 1067 | −15.421 | 6.067 | 48.484 | 1.00 | 47.05 | A |
| 440 | C | GLY | A | 1067 | −16.057 | 7.204 | 49.282 | 1.00 | 47.29 | A |
| 441 | O | GLY | A | 1067 | −17.138 | 7.044 | 49.806 | 1.00 | 48.11 | A |
| 442 | N | ARG | A | 1068 | −15.479 | 8.405 | 49.360 | 1.00 | 47.32 | A |
| 443 | CA | ARG | A | 1068 | −16.168 | 9.406 | 50.205 | 1.00 | 49.64 | A |
| 444 | CB | ARG | A | 1068 | −15.094 | 10.122 | 50.989 | 1.00 | 49.69 | A |
| 445 | CG | ARG | A | 1068 | −14.137 | 9.211 | 51.539 | 1.00 | 42.24 | A |
| 446 | CD | ARG | A | 1068 | −14.887 | 8.456 | 52.478 | 1.00 | 41.23 | A |
| 447 | NE | ARG | A | 1068 | −13.990 | 7.748 | 53.372 | 1.00 | 51.66 | A |
| 448 | CZ | ARG | A | 1068 | −14.026 | 7.899 | 54.695 | 1.00 | 56.52 | A |
| 449 | NH1 | ARG | A | 1068 | −15.020 | 8.603 | 55.411 | 1.00 | 49.15 | A |
| 450 | NH2 | ARG | A | 1068 | −13.079 | 7.279 | 55.312 | 1.00 | 52.95 | A |
| 451 | C | ARG | A | 1068 | −16.736 | 10.307 | 49.135 | 1.00 | 51.50 | A |
| 452 | O | ARG | A | 1068 | −16.215 | 10.169 | 48.004 | 1.00 | 53.24 | A |
| 453 | N | TYR | A | 1069 | −17.692 | 11.194 | 49.436 | 1.00 | 47.45 | A |
| 454 | CA | TYR | A | 1069 | −18.345 | 11.885 | 48.405 | 1.00 | 47.50 | A |
| 455 | CB | TYR | A | 1069 | −19.789 | 11.932 | 48.778 | 1.00 | 51.64 | A |
| 456 | CG | TYR | A | 1069 | −20.442 | 10.525 | 48.640 | 1.00 | 57.11 | A |
| 457 | CD1 | TYR | A | 1069 | −21.287 | 10.272 | 47.577 | 1.00 | 61.24 | A |
| 458 | CE1 | TYR | A | 1069 | −21.858 | 9.071 | 47.417 | 1.00 | 65.65 | A |
| 459 | CD2 | TYR | A | 1069 | −20.111 | 9.439 | 49.482 | 1.00 | 62.48 | A |
| 460 | CE2 | TYR | A | 1069 | −20.652 | 8.187 | 49.291 | 1.00 | 60.12 | A |
| 461 | CZ | TYR | A | 1069 | −21.558 | 8.006 | 48.251 | 1.00 | 62.30 | A |
| 462 | OH | TYR | A | 1069 | −22.239 | 6.815 | 47.917 | 1.00 | 57.76 | A |
| 463 | C | TYR | A | 1069 | −17.727 | 13.245 | 48.152 | 1.00 | 48.56 | A |
| 464 | O | TYR | A | 1069 | −16.486 | 13.308 | 47.842 | 1.00 | 51.36 | A |
| 465 | N | LEU | A | 1070 | −18.462 | 14.362 | 48.284 | 1.00 | 43.32 | A |
| 466 | CA | LEU | A | 1070 | −17.870 | 15.704 | 48.089 | 1.00 | 42.37 | A |
| 467 | CB | LEU | A | 1070 | −18.070 | 16.149 | 46.581 | 1.00 | 43.29 | A |
| 468 | CG | LEU | A | 1070 | −17.221 | 17.196 | 45.814 | 1.00 | 41.01 | A |
| 469 | CD1 | LEU | A | 1070 | −15.633 | 17.104 | 45.982 | 1.00 | 29.40 | A |
| 470 | CD2 | LEU | A | 1070 | −17.603 | 17.432 | 44.361 | 1.00 | 36.72 | A |
| 471 | C | LEU | A | 1070 | −18.839 | 16.565 | 48.804 | 1.00 | 44.22 | A |
| 472 | O | LEU | A | 1070 | −20.013 | 16.504 | 48.428 | 1.00 | 46.64 | A |
| 473 | N | ALA | A | 1071 | −18.503 | 17.352 | 49.823 | 1.00 | 44.19 | A |
| 474 | CA | ALA | A | 1071 | −19.594 | 18.199 | 50.357 | 1.00 | 44.99 | A |
| 475 | CB | ALA | A | 1071 | −19.924 | 17.889 | 51.767 | 1.00 | 42.78 | A |
| 476 | C | ALA | A | 1071 | −19.234 | 19.643 | 50.190 | 1.00 | 46.02 | A |
| 477 | O | ALA | A | 1071 | −17.982 | 20.019 | 50.094 | 1.00 | 46.31 | A |
| 478 | N | ALA | A | 1072 | −20.254 | 20.455 | 50.242 | 1.00 | 46.45 | A |
| 479 | CA | ALA | A | 1072 | −19.987 | 21.889 | 50.285 | 1.00 | 50.39 | A |
| 480 | CB | ALA | A | 1072 | −20.185 | 22.467 | 48.958 | 1.00 | 50.40 | A |
| 481 | C | ALA | A | 1072 | −20.835 | 22.626 | 51.331 | 1.00 | 53.13 | A |
| 482 | O | ALA | A | 1072 | −22.076 | 22.752 | 51.181 | 1.00 | 55.34 | A |
| 483 | N | ASP | A | 1073 | −20.224 | 23.094 | 52.435 | 1.00 | 53.60 | A |
| 484 | CA | ASP | A | 1073 | −21.124 | 23.722 | 53.441 | 1.00 | 52.65 | A |
| 485 | CB | ASP | A | 1073 | −20.677 | 23.401 | 54.891 | 1.00 | 53.33 | A |
| 486 | CG | ASP | A | 1073 | −19.193 | 23.084 | 54.999 | 1.00 | 56.93 | A |
| 487 | OD1 | ASP | A | 1073 | −18.533 | 23.310 | 56.080 | 1.00 | 61.74 | A |
| 488 | OD2 | ASP | A | 1073 | −18.668 | 22.632 | 53.932 | 1.00 | 62.41 | A |
| 489 | C | ASP | A | 1073 | −21.368 | 25.224 | 53.162 | 1.00 | 52.27 | A |
| 490 | O | ASP | A | 1073 | −20.776 | 25.830 | 52.346 | 1.00 | 51.94 | A |
| 491 | N | LYS | A | 1074 | −22.289 | 25.845 | 53.857 | 1.00 | 54.57 | A |
| 492 | CA | LYS | A | 1074 | −22.733 | 27.147 | 53.526 | 1.00 | 53.80 | A |
| 493 | CB | LYS | A | 1074 | −23.996 | 27.448 | 54.360 | 1.00 | 56.45 | A |
| 494 | CG | LYS | A | 1074 | −25.470 | 27.116 | 53.659 | 1.00 | 58.21 | A |
| 495 | CD | LYS | A | 1074 | −26.664 | 27.495 | 54.668 | 1.00 | 54.48 | A |
| 496 | CE | LYS | A | 1074 | −28.006 | 27.670 | 53.988 | 1.00 | 49.89 | A |
| 497 | NZ | LYS | A | 1074 | −28.898 | 28.147 | 54.991 | 1.00 | 53.66 | A |
| 498 | C | LYS | A | 1074 | −21.587 | 28.107 | 53.702 | 1.00 | 53.97 | A |
| 499 | O | LYS | A | 1074 | −21.744 | 29.261 | 53.893 | 1.00 | 54.47 | A |
| 500 | N | ASP | A | 1075 | −20.380 | 27.632 | 53.542 | 1.00 | 55.26 | A |
| 501 | CA | ASP | A | 1075 | −19.256 | 28.540 | 53.600 | 1.00 | 53.97 | A |
| 502 | CB | ASP | A | 1075 | −18.538 | 28.249 | 54.943 | 1.00 | 54.71 | A |
| 503 | CG | ASP | A | 1075 | −19.426 | 28.620 | 56.078 | 1.00 | 55.41 | A |
| 504 | OD1 | ASP | A | 1075 | −20.372 | 27.857 | 56.312 | 1.00 | 58.04 | A |
| 505 | OD2 | ASP | A | 1075 | −19.275 | 29.774 | 56.571 | 1.00 | 54.16 | A |
| 506 | C | ASP | A | 1075 | −18.238 | 28.425 | 52.565 | 1.00 | 54.10 | A |
| 507 | O | ASP | A | 1075 | −17.161 | 29.039 | 52.837 | 1.00 | 55.78 | A |
| 508 | N | GLY | A | 1076 | −18.451 | 27.546 | 51.520 | 1.00 | 52.33 | A |
| 509 | CA | GLY | A | 1076 | −17.481 | 27.255 | 50.455 | 1.00 | 50.05 | A |
| 510 | C | GLY | A | 1076 | −16.520 | 26.162 | 50.876 | 1.00 | 51.21 | A |
| 511 | O | GLY | A | 1076 | −15.550 | 25.813 | 50.218 | 1.00 | 52.93 | A |
| 512 | N | ASN | A | 1077 | −16.641 | 25.642 | 52.049 | 1.00 | 48.90 | A |
| 513 | CA | ASN | A | 1077 | −15.745 | 24.516 | 52.235 | 1.00 | 49.47 | A |
| 514 | CB | ASN | A | 1077 | −15.991 | 24.020 | 53.651 | 1.00 | 50.95 | A |
| 515 | CG | ASN | A | 1077 | −16.396 | 25.019 | 54.441 | 1.00 | 46.43 | A |
| 516 | OD1 | ASN | A | 1077 | −15.581 | 25.912 | 54.708 | 1.00 | 56.36 | A |
| 517 | ND2 | ASN | A | 1077 | −17.648 | 25.046 | 54.749 | 1.00 | 48.43 | A |
| 518 | C | ASN | A | 1077 | −15.921 | 23.256 | 51.410 | 1.00 | 49.14 | A |
| 519 | O | ASN | A | 1077 | −17.005 | 22.569 | 51.480 | 1.00 | 50.93 | A |
| 520 | N | VAL | A | 1078 | −14.832 | 22.791 | 50.849 | 1.00 | 49.40 | A |
| 521 | CA | VAL | A | 1078 | −14.872 | 21.472 | 50.162 | 1.00 | 48.53 | A |
| 522 | CB | VAL | A | 1078 | −14.553 | 21.475 | 48.652 | 1.00 | 46.65 | A |
| 523 | CG1 | VAL | A | 1078 | −13.935 | 22.562 | 48.333 | 1.00 | 30.89 | A |
| 524 | CG2 | VAL | A | 1078 | −13.771 | 20.086 | 48.225 | 1.00 | 45.09 | A |
| 525 | C | VAL | A | 1078 | −14.285 | 20.270 | 50.836 | 1.00 | 51.34 | A |
| 526 | O | VAL | A | 1078 | −13.058 | 20.147 | 51.063 | 1.00 | 54.53 | A |
| 527 | N | THR | A | 1079 | −15.203 | 19.379 | 51.179 | 1.00 | 51.30 | A |
| 528 | CA | THR | A | 1079 | −14.791 | 18.183 | 51.855 | 1.00 | 49.51 | A |
| 529 | CB | THR | A | 1079 | −15.281 | 18.180 | 53.248 | 1.00 | 48.77 | A |
| 530 | OG1 | THR | A | 1079 | −16.519 | 18.910 | 53.302 | 1.00 | 48.75 | A |
| 531 | CG2 | THR | A | 1079 | −14.232 | 18.816 | 54.155 | 1.00 | 43.68 | A |
| 532 | C | THR | A | 1079 | −15.313 | 17.001 | 51.032 | 1.00 | 51.17 | A |
| 533 | O | THR | A | 1079 | −16.382 | 17.061 | 50.444 | 1.00 | 52.70 | A |
| 534 | N | CYS | A | 1080 | −14.398 | 16.068 | 50.841 | 1.00 | 49.63 | A |
| 535 | CA | CYS | A | 1080 | −14.615 | 14.818 | 50.382 | 1.00 | 49.93 | A |
| 536 | CB | CYS | A | 1080 | −13.731 | 14.719 | 49.248 | 1.00 | 45.32 | A |
| 537 | SG | CYS | A | 1080 | −14.144 | 13.142 | 48.423 | 1.00 | 45.31 | A |
| 538 | C | CYS | A | 1080 | −14.236 | 13.686 | 51.436 | 1.00 | 51.02 | A |
| 539 | O | CYS | A | 1080 | −13.245 | 13.033 | 51.292 | 1.00 | 52.54 | A |
| 540 | N | GLU | A | 1081 | −14.968 | 13.521 | 52.504 | 1.00 | 51.75 | A |
| 541 | CA | GLU | A | 1081 | −14.573 | 12.634 | 53.641 | 1.00 | 54.09 | A |
| 542 | CB | GLU | A | 1081 | −13.912 | 13.418 | 54.814 | 1.00 | 54.32 | A |
| 543 | CG | GLU | A | 1081 | −14.881 | 14.205 | 55.784 | 1.00 | 54.39 | A |
| 544 | CD | GLU | A | 1081 | −14.190 | 15.533 | 56.249 | 1.00 | 57.02 | A |
| 545 | OE1 | GLU | A | 1081 | −14.909 | 16.473 | 56.764 | 1.00 | 54.50 | A |
| 546 | OE2 | GLU | A | 1081 | −12.941 | 15.675 | 55.988 | 1.00 | 55.50 | A |
| 547 | C | GLU | A | 1081 | −15.774 | 11.825 | 54.256 | 1.00 | 55.38 | A |
| 548 | O | GLU | A | 1081 | −15.530 | 10.661 | 54.767 | 1.00 | 54.03 | A |
| 549 | N | ARG | A | 1082 | −17.010 | 12.437 | 54.201 | 1.00 | 55.32 | A |
| 550 | CA | ARG | A | 1082 | −18.255 | 11.729 | 54.441 | 1.00 | 57.01 | A |
| 551 | CB | ARG | A | 1082 | −19.383 | 12.668 | 54.716 | 1.00 | 59.69 | A |
| 552 | CG | ARG | A | 1082 | −19.836 | 13.586 | 53.585 | 1.00 | 64.08 | A |
| 553 | CD | ARG | A | 1082 | −20.463 | 14.900 | 54.257 | 1.00 | 65.95 | A |
| 554 | NE | ARG | A | 1082 | −21.770 | 14.552 | 54.770 | 1.00 | 72.82 | A |
| 555 | CZ | ARG | A | 1082 | −22.936 | 15.229 | 54.610 | 1.00 | 72.48 | A |
| 556 | NH1 | ARG | A | 1082 | −23.014 | 16.412 | 53.925 | 1.00 | 57.84 | A |
| 557 | NH2 | ARG | A | 1082 | −24.059 | 14.666 | 55.174 | 1.00 | 71.91 | A |
| 558 | C | ARG | A | 1082 | −18.723 | 10.668 | 53.476 | 1.00 | 57.07 | A |
| 559 | O | ARG | A | 1082 | −18.826 | 10.866 | 52.258 | 1.00 | 56.91 | A |
| 560 | N | GLU | A | 1083 | −18.973 | 9.509 | 54.096 | 1.00 | 56.68 | A |
| 561 | CA | GLU | A | 1083 | −19.322 | 8.214 | 53.428 | 1.00 | 56.23 | A |
| 562 | CB | GLU | A | 1083 | −18.834 | 7.097 | 54.238 | 1.00 | 52.11 | A |
| 563 | CG | GLU | A | 1083 | −19.304 | 7.301 | 55.604 | 1.00 | 56.40 | A |
| 564 | CD | GLU | A | 1083 | −18.842 | 6.215 | 56.509 | 1.00 | 65.36 | A |
| 565 | OE1 | GLU | A | 1083 | −17.525 | 6.169 | 56.607 | 1.00 | 57.98 | A |
| 566 | OE2 | GLU | A | 1083 | −19.784 | 5.386 | 57.003 | 1.00 | 62.74 | A |
| 567 | C | GLU | A | 1083 | −20.833 | 8.013 | 53.236 | 1.00 | 57.67 | A |
| 568 | O | GLU | A | 1083 | −21.228 | 7.201 | 52.398 | 1.00 | 57.89 | A |
| 569 | N | VAL | A | 1084 | −21.647 | 8.822 | 53.938 | 1.00 | 57.79 | A |
| 570 | CA | VAL | A | 1084 | −23.007 | 8.924 | 53.625 | 1.00 | 58.47 | A |
| 571 | CB | VAL | A | 1084 | −23.808 | 8.255 | 54.742 | 1.00 | 60.91 | A |
| 572 | CG1 | VAL | A | 1084 | −25.381 | 8.538 | 54.673 | 1.00 | 59.38 | A |
| 573 | CG2 | VAL | A | 1084 | −23.603 | 6.695 | 54.729 | 1.00 | 60.24 | A |
| 574 | C | VAL | A | 1084 | −23.406 | 10.401 | 53.399 | 1.00 | 59.76 | A |
| 575 | O | VAL | A | 1084 | −23.515 | 11.177 | 54.316 | 1.00 | 61.89 | A |
| 576 | N | PRO | A | 1085 | −23.538 | 10.797 | 52.163 | 1.00 | 59.08 | A |
| 577 | CD | PRO | A | 1085 | −22.730 | 10.194 | 51.109 | 1.00 | 61.69 | A |
| 578 | CA | PRO | A | 1085 | −24.347 | 11.754 | 51.516 | 1.00 | 59.94 | A |
| 579 | CB | PRO | A | 1085 | −24.994 | 10.855 | 50.402 | 1.00 | 58.86 | A |
| 580 | CG | PRO | A | 1085 | −23.825 | 10.041 | 49.937 | 1.00 | 58.73 | A |
| 581 | C | PRO | A | 1085 | −25.456 | 12.503 | 52.260 | 1.00 | 59.13 | A |
| 582 | O | PRO | A | 1085 | −26.461 | 11.867 | 52.450 | 1.00 | 59.92 | A |
| 583 | N | GLY | A | 1086 | −25.288 | 13.816 | 52.564 | 1.00 | 56.69 | A |
| 584 | CA | GLY | A | 1086 | −26.169 | 14.642 | 53.479 | 1.00 | 55.27 | A |
| 585 | C | GLY | A | 1086 | −26.800 | 15.722 | 52.627 | 1.00 | 53.00 | A |
| 586 | O | GLY | A | 1086 | −26.767 | 15.511 | 51.472 | 1.00 | 55.42 | A |
| 587 | N | PRO | A | 1087 | −27.472 | 16.779 | 53.125 | 1.00 | 52.87 | A |
| 588 | CD | PRO | A | 1087 | −28.323 | 16.922 | 54.328 | 1.00 | 55.67 | A |
| 589 | CA | PRO | A | 1087 | −27.774 | 17.905 | 52.192 | 1.00 | 53.56 | A |
| 590 | CB | PRO | A | 1087 | −28.574 | 18.893 | 53.004 | 1.00 | 50.99 | A |
| 591 | CG | PRO | A | 1087 | −29.386 | 17.981 | 53.814 | 1.00 | 55.52 | A |
| 592 | C | PRO | A | 1087 | −26.612 | 18.566 | 51.625 | 1.00 | 55.63 | A |
| 593 | O | PRO | A | 1087 | −26.749 | 19.384 | 50.621 | 1.00 | 58.05 | A |
| 594 | N | ASP | A | 1088 | −25.447 | 18.219 | 52.158 | 1.00 | 56.01 | A |
| 595 | CA | ASP | A | 1088 | −24.351 | 18.953 | 51.701 | 1.00 | 55.34 | A |
| 596 | CB | ASP | A | 1088 | −23.579 | 19.524 | 52.870 | 1.00 | 58.96 | A |
| 597 | CG | ASP | A | 1088 | −23.940 | 20.939 | 53.069 | 1.00 | 62.44 | A |
| 598 | OD1 | ASP | A | 1088 | −24.435 | 21.394 | 54.131 | 1.00 | 67.96 | A |
| 599 | OD2 | ASP | A | 1088 | −23.904 | 21.576 | 52.011 | 1.00 | 69.27 | A |
| 600 | C | ASP | A | 1088 | −23.594 | 18.346 | 50.594 | 1.00 | 54.12 | A |
| 601 | O | ASP | A | 1088 | −22.874 | 19.068 | 49.882 | 1.00 | 56.36 | A |
| 602 | N | CYS | A | 1089 | −23.825 | 17.097 | 50.300 | 1.00 | 50.47 | A |
| 603 | CA | CYS | A | 1089 | −23.078 | 16.490 | 49.218 | 1.00 | 50.41 | A |
| 604 | CB | CYS | A | 1089 | −22.851 | 15.027 | 49.651 | 1.00 | 49.53 | A |
| 605 | SG | CYS | A | 1089 | −21.907 | 14.459 | 51.307 | 1.00 | 54.68 | A |
| 606 | C | CYS | A | 1089 | −23.821 | 16.606 | 47.722 | 1.00 | 52.20 | A |
| 607 | O | CYS | A | 1089 | −23.338 | 15.997 | 46.746 | 1.00 | 51.41 | A |
| 608 | N | ARG | A | 1090 | −24.959 | 17.346 | 47.560 | 1.00 | 50.29 | A |
| 609 | CA | ARG | A | 1090 | −25.771 | 17.377 | 46.269 | 1.00 | 51.09 | A |
| 610 | CB | ARG | A | 1090 | −27.367 | 17.383 | 46.442 | 1.00 | 52.39 | A |
| 611 | CG | ARG | A | 1090 | −28.217 | 16.731 | 47.699 | 1.00 | 52.01 | A |
| 612 | CD | ARG | A | 1090 | −28.857 | 15.473 | 47.127 | 1.00 | 58.91 | A |
| 613 | NE | ARG | A | 1090 | −30.299 | 15.291 | 47.184 | 1.00 | 58.93 | A |
| 614 | CZ | ARG | A | 1090 | −31.230 | 16.250 | 47.029 | 1.00 | 66.98 | A |
| 615 | NH1 | ARG | A | 1090 | −30.940 | 17.558 | 46.811 | 1.00 | 64.17 | A |
| 616 | NH2 | ARG | A | 1090 | −32.515 | 15.907 | 47.139 | 1.00 | 67.07 | A |
| 617 | C | ARG | A | 1090 | −25.561 | 18.585 | 45.321 | 1.00 | 49.48 | A |
| 618 | O | ARG | A | 1090 | −26.102 | 19.672 | 45.545 | 1.00 | 46.66 | A |
| 619 | N | PHE | A | 1091 | −24.901 | 18.326 | 44.207 | 1.00 | 49.55 | A |
| 620 | CA | PHE | A | 1091 | −24.622 | 19.340 | 43.121 | 1.00 | 46.71 | A |
| 621 | CB | PHE | A | 1091 | −23.231 | 19.295 | 42.784 | 1.00 | 48.06 | A |
| 622 | CG | PHE | A | 1091 | −22.352 | 19.351 | 43.983 | 1.00 | 52.98 | A |
| 623 | CD1 | PHE | A | 1091 | −21.779 | 20.553 | 44.378 | 1.00 | 54.19 | A |
| 624 | CD2 | PHE | A | 1091 | −22.135 | 18.228 | 44.717 | 1.00 | 54.06 | A |
| 625 | CE1 | PHE | A | 1091 | −20.968 | 20.640 | 45.519 | 1.00 | 56.13 | A |
| 626 | CE2 | PHE | A | 1091 | −21.380 | 18.280 | 45.847 | 1.00 | 60.51 | A |
| 627 | CZ | PHE | A | 1091 | −20.744 | 19.494 | 46.248 | 1.00 | 60.12 | A |
| 628 | C | PHE | A | 1091 | −25.443 | 19.202 | 41.868 | 1.00 | 43.36 | A |
| 629 | O | PHE | A | 1091 | −26.309 | 18.445 | 41.768 | 1.00 | 42.69 | A |
| 630 | N | LEU | A | 1092 | −25.256 | 20.034 | 40.928 | 1.00 | 43.49 | A |
| 631 | CA | LEU | A | 1092 | −26.171 | 20.040 | 39.772 | 1.00 | 43.00 | A |
| 632 | CB | LEU | A | 1092 | −27.108 | 21.166 | 39.845 | 1.00 | 41.09 | A |
| 633 | CG | LEU | A | 1092 | −28.306 | 21.058 | 40.731 | 1.00 | 39.34 | A |
| 634 | CD1 | LEU | A | 1092 | −28.414 | 22.398 | 41.184 | 1.00 | 38.60 | A |
| 635 | CD2 | LEU | A | 1092 | −29.595 | 20.886 | 40.162 | 1.00 | 25.44 | A |
| 636 | C | LEU | A | 1092 | −25.201 | 20.389 | 38.766 | 1.00 | 42.42 | A |
| 637 | O | LEU | A | 1092 | −24.648 | 21.378 | 38.968 | 1.00 | 39.92 | A |
| 638 | N | ILE | A | 1093 | −24.872 | 19.462 | 37.842 | 1.00 | 45.45 | A |
| 639 | CA | ILE | A | 1093 | −24.237 | 19.706 | 36.518 | 1.00 | 46.07 | A |
| 640 | CB | ILE | A | 1093 | −24.448 | 18.594 | 35.561 | 1.00 | 44.66 | A |
| 641 | CG2 | ILE | A | 1093 | −23.545 | 18.812 | 34.302 | 1.00 | 43.88 | A |
| 642 | CG1 | ILE | A | 1093 | −24.033 | 17.206 | 36.167 | 1.00 | 44.44 | A |
| 643 | CD1 | ILE | A | 1093 | −23.461 | 17.228 | 37.476 | 1.00 | 46.13 | A |
| 644 | C | ILE | A | 1093 | −24.903 | 20.853 | 35.885 | 1.00 | 48.03 | A |
| 645 | O | ILE | A | 1093 | −26.155 | 20.922 | 35.966 | 1.00 | 50.68 | A |
| 646 | N | VAL | A | 1094 | −24.116 | 21.818 | 35.391 | 1.00 | 49.43 | A |
| 647 | CA | VAL | A | 1094 | −24.768 | 22.791 | 34.466 | 1.00 | 55.65 | A |
| 648 | CB | VAL | A | 1094 | −25.009 | 24.187 | 35.170 | 1.00 | 56.33 | A |
| 649 | CG1 | VAL | A | 1094 | −26.351 | 24.747 | 34.712 | 1.00 | 57.71 | A |
| 650 | CG2 | VAL | A | 1094 | −25.137 | 23.974 | 36.779 | 1.00 | 55.27 | A |
| 651 | C | VAL | A | 1094 | −24.158 | 22.834 | 32.993 | 1.00 | 56.94 | A |
| 652 | O | VAL | A | 1094 | −23.191 | 23.546 | 32.749 | 1.00 | 60.96 | A |
| 653 | N | ALA | A | 1095 | −24.592 | 22.015 | 32.030 | 1.00 | 55.92 | A |
| 654 | CA | ALA | A | 1095 | −23.968 | 22.172 | 30.683 | 1.00 | 56.02 | A |
| 655 | CB | ALA | A | 1095 | −24.415 | 21.097 | 29.689 | 1.00 | 55.70 | A |
| 656 | C | ALA | A | 1095 | −24.075 | 23.515 | 30.021 | 1.00 | 55.99 | A |
| 657 | O | ALA | A | 1095 | −25.174 | 24.238 | 29.973 | 1.00 | 56.99 | A |
| 658 | N | HIS | A | 1096 | −22.947 | 23.818 | 29.417 | 1.00 | 57.87 | A |
| 659 | CA | HIS | A | 1096 | −22.677 | 25.191 | 28.954 | 1.00 | 61.82 | A |
| 660 | CB | HIS | A | 1096 | −21.590 | 25.857 | 29.751 | 1.00 | 60.24 | A |
| 661 | CG | HIS | A | 1096 | −22.112 | 26.381 | 31.032 | 1.00 | 59.56 | A |
| 662 | CD2 | HIS | A | 1096 | −21.537 | 26.509 | 32.246 | 1.00 | 64.07 | A |
| 663 | ND1 | HIS | A | 1096 | −23.420 | 26.803 | 31.164 | 1.00 | 52.40 | A |
| 664 | CE1 | HIS | A | 1096 | −23.617 | 27.199 | 32.399 | 1.00 | 64.14 | A |
| 665 | NE2 | HIS | A | 1096 | −22.491 | 27.021 | 33.087 | 1.00 | 67.16 | A |
| 666 | C | HIS | A | 1096 | −22.411 | 25.271 | 27.475 | 1.00 | 64.40 | A |
| 667 | O | HIS | A | 1096 | −21.172 | 25.236 | 27.020 | 1.00 | 64.38 | A |
| 668 | N | ASP | A | 1097 | −23.595 | 25.237 | 26.741 | 1.00 | 65.38 | A |
| 669 | CA | ASP | A | 1097 | −23.670 | 25.448 | 25.350 | 1.00 | 66.57 | A |
| 670 | CB | ASP | A | 1097 | −24.030 | 26.906 | 24.999 | 1.00 | 66.95 | A |
| 671 | CG | ASP | A | 1097 | −25.524 | 27.280 | 25.285 | 1.00 | 71.79 | A |
| 672 | OD1 | ASP | A | 1097 | −25.884 | 28.224 | 24.572 | 1.00 | 73.85 | A |
| 673 | OD2 | ASP | A | 1097 | −26.311 | 26.699 | 26.115 | 1.00 | 71.77 | A |
| 674 | C | ASP | A | 1097 | −22.246 | 25.282 | 25.111 | 1.00 | 65.98 | A |
| 675 | O | ASP | A | 1097 | −21.658 | 24.264 | 25.456 | 1.00 | 69.86 | A |
| 676 | N | ASP | A | 1098 | −21.594 | 26.308 | 24.693 | 1.00 | 65.57 | A |
| 677 | CA | ASP | A | 1098 | −20.237 | 26.117 | 24.352 | 1.00 | 65.74 | A |
| 678 | CB | ASP | A | 1098 | −20.114 | 26.985 | 23.143 | 1.00 | 67.00 | A |
| 679 | CG | ASP | A | 1098 | −21.226 | 26.733 | 22.063 | 1.00 | 73.26 | A |
| 680 | OD1 | ASP | A | 1098 | −22.380 | 26.278 | 22.307 | 1.00 | 73.41 | A |
| 681 | OD2 | ASP | A | 1098 | −20.842 | 27.090 | 20.882 | 1.00 | 78.59 | A |
| 682 | C | ASP | A | 1098 | −18.993 | 26.354 | 25.415 | 1.00 | 65.81 | A |
| 683 | O | ASP | A | 1098 | −18.006 | 27.082 | 25.239 | 1.00 | 65.17 | A |
| 684 | N | GLY | A | 1099 | −18.793 | 25.501 | 26.471 | 1.00 | 64.28 | A |
| 685 | CA | GLY | A | 1099 | −17.621 | 25.875 | 27.314 | 1.00 | 63.47 | A |
| 686 | C | GLY | A | 1099 | −17.509 | 24.473 | 27.857 | 1.00 | 63.88 | A |
| 687 | O | GLY | A | 1099 | −17.146 | 23.490 | 27.059 | 1.00 | 63.26 | A |
| 688 | N | ARG | A | 1100 | −17.991 | 24.317 | 29.109 | 1.00 | 61.08 | A |
| 689 | CA | ARG | A | 1100 | −17.789 | 23.096 | 29.897 | 1.00 | 57.01 | A |
| 690 | CB | ARG | A | 1100 | −16.519 | 23.407 | 30.720 | 1.00 | 58.35 | A |
| 691 | CG | ARG | A | 1100 | −15.173 | 23.592 | 29.838 | 1.00 | 53.42 | A |
| 692 | CD | ARG | A | 1100 | −14.106 | 24.004 | 30.799 | 1.00 | 52.12 | A |
| 693 | NE | ARG | A | 1100 | −14.623 | 25.343 | 30.934 | 1.00 | 52.10 | A |
| 694 | CZ | ARG | A | 1100 | −13.882 | 26.433 | 31.028 | 1.00 | 46.76 | A |
| 695 | NH1 | ARG | A | 1100 | −12.560 | 26.304 | 31.190 | 1.00 | 42.27 | A |
| 696 | NH2 | ARG | A | 1100 | −14.529 | 27.596 | 31.061 | 1.00 | 36.94 | A |
| 697 | C | ARG | A | 1100 | −19.031 | 22.782 | 30.752 | 1.00 | 55.62 | A |
| 698 | O | ARG | A | 1100 | −20.107 | 22.924 | 30.231 | 1.00 | 52.91 | A |
| 699 | N | TRP | A | 1101 | −18.887 | 22.309 | 32.004 | 1.00 | 54.88 | A |
| 700 | CA | TRP | A | 1101 | −20.029 | 22.095 | 32.989 | 1.00 | 57.27 | A |
| 701 | CB | TRP | A | 1101 | −19.758 | 20.760 | 33.656 | 1.00 | 58.05 | A |
| 702 | CG | TRP | A | 1101 | −20.439 | 19.614 | 32.937 | 1.00 | 64.92 | A |
| 703 | CD2 | TRP | A | 1101 | −20.578 | 18.224 | 33.403 | 1.00 | 69.91 | A |
| 704 | CE2 | TRP | A | 1101 | −21.382 | 17.542 | 32.458 | 1.00 | 69.98 | A |
| 705 | CE3 | TRP | A | 1101 | −20.144 | 17.521 | 34.537 | 1.00 | 68.13 | A |
| 706 | CD1 | TRP | A | 1101 | −21.085 | 19.673 | 31.724 | 1.00 | 65.96 | A |
| 707 | NE1 | TRP | A | 1101 | −21.685 | 18.438 | 31.451 | 1.00 | 69.40 | A |
| 708 | CZ2 | TRP | A | 1101 | −21.742 | 16.211 | 32.625 | 1.00 | 62.24 | A |
| 709 | CZ3 | TRP | A | 1101 | −20.508 | 16.184 | 34.689 | 1.00 | 62.33 | A |
| 710 | CH2 | TRP | A | 1101 | −21.297 | 15.558 | 33.753 | 1.00 | 62.00 | A |
| 711 | C | TRP | A | 1101 | −20.284 | 23.258 | 34.108 | 1.00 | 55.94 | A |
| 712 | O | TRP | A | 1101 | −19.585 | 24.293 | 34.043 | 1.00 | 53.62 | A |
| 713 | N | SER | A | 1102 | −21.262 | 23.132 | 35.057 | 1.00 | 55.99 | A |
| 714 | CA | SER | A | 1102 | −21.056 | 23.830 | 36.411 | 1.00 | 58.45 | A |
| 715 | CB | SER | A | 1102 | −21.300 | 25.392 | 36.386 | 1.00 | 58.51 | A |
| 716 | OG | SER | A | 1102 | −20.179 | 26.219 | 35.938 | 1.00 | 54.55 | A |
| 717 | C | SER | A | 1102 | −21.641 | 23.306 | 37.756 | 1.00 | 59.31 | A |
| 718 | O | SER | A | 1102 | −22.782 | 23.673 | 38.140 | 1.00 | 59.33 | A |
| 719 | N | LEU | A | 1103 | −20.811 | 22.573 | 38.501 | 1.00 | 59.63 | A |
| 720 | CA | LEU | A | 1103 | −21.107 | 22.083 | 39.892 | 1.00 | 60.50 | A |
| 721 | CB | LEU | A | 1103 | −19.933 | 21.276 | 40.443 | 1.00 | 59.64 | A |
| 722 | CG | LEU | A | 1103 | −19.879 | 19.787 | 40.183 | 1.00 | 59.22 | A |
| 723 | CD1 | LEU | A | 1103 | −21.124 | 19.102 | 40.780 | 1.00 | 52.84 | A |
| 724 | CD2 | LEU | A | 1103 | −19.715 | 19.555 | 38.588 | 1.00 | 59.39 | A |
| 725 | C | LEU | A | 1103 | −21.493 | 23.167 | 40.937 | 1.00 | 61.43 | A |
| 726 | O | LEU | A | 1103 | −20.610 | 23.691 | 41.723 | 1.00 | 62.71 | A |
| 727 | N | GLN | A | 1104 | −22.784 | 23.532 | 40.876 | 1.00 | 60.17 | A |
| 728 | CA | GLN | A | 1104 | −23.431 | 24.334 | 41.845 | 1.00 | 60.57 | A |
| 729 | CB | GLN | A | 1104 | −24.451 | 25.083 | 41.105 | 1.00 | 58.35 | A |
| 730 | CG | GLN | A | 1104 | −25.497 | 25.686 | 42.051 | 1.00 | 60.90 | A |
| 731 | CD | GLN | A | 1104 | −26.876 | 25.893 | 41.317 | 1.00 | 56.31 | A |
| 732 | OE1 | GLN | A | 1104 | −26.976 | 25.702 | 40.037 | 1.00 | 60.62 | A |
| 733 | NE2 | GLN | A | 1104 | −27.937 | 26.157 | 42.098 | 1.00 | 36.93 | A |
| 734 | C | GLN | A | 1104 | −24.103 | 23.480 | 42.996 | 1.00 | 61.59 | A |
| 735 | O | GLN | A | 1104 | −24.889 | 22.534 | 42.712 | 1.00 | 63.63 | A |
| 736 | N | SER | A | 1105 | −23.868 | 23.840 | 44.268 | 1.00 | 60.16 | A |
| 737 | CA | SER | A | 1105 | −24.666 | 23.339 | 45.405 | 1.00 | 56.98 | A |
| 738 | CB | SER | A | 1105 | −23.931 | 23.612 | 46.729 | 1.00 | 57.37 | A |
| 739 | OG | SER | A | 1105 | −24.702 | 23.243 | 47.875 | 1.00 | 61.71 | A |
| 740 | C | SER | A | 1105 | −26.028 | 23.983 | 45.391 | 1.00 | 56.48 | A |
| 741 | O | SER | A | 1105 | −26.273 | 25.192 | 45.088 | 1.00 | 58.43 | A |
| 742 | N | GLU | A | 1106 | −26.979 | 23.170 | 45.716 | 1.00 | 55.66 | A |
| 743 | CA | GLU | A | 1106 | −28.374 | 23.476 | 45.409 | 1.00 | 52.93 | A |
| 744 | CB | GLU | A | 1106 | −28.988 | 22.165 | 45.019 | 1.00 | 52.65 | A |
| 745 | CG | GLU | A | 1106 | −30.368 | 22.165 | 45.360 | 1.00 | 49.18 | A |
| 746 | CD | GLU | A | 1106 | −30.895 | 20.778 | 45.549 | 1.00 | 49.36 | A |
| 747 | OE1 | GLU | A | 1106 | −30.180 | 19.914 | 46.140 | 1.00 | 45.18 | A |
| 748 | OE2 | GLU | A | 1106 | −32.056 | 20.570 | 45.097 | 1.00 | 50.68 | A |
| 749 | C | GLU | A | 1106 | −29.119 | 23.889 | 46.638 | 1.00 | 53.58 | A |
| 750 | O | GLU | A | 1106 | −30.186 | 24.534 | 46.521 | 1.00 | 49.78 | A |
| 751 | N | ALA | A | 1107 | −28.586 | 23.402 | 47.799 | 1.00 | 52.93 | A |
| 752 | CA | ALA | A | 1107 | −29.040 | 23.797 | 49.068 | 1.00 | 54.59 | A |
| 753 | CB | ALA | A | 1107 | −28.115 | 23.238 | 50.151 | 1.00 | 54.41 | A |
| 754 | C | ALA | A | 1107 | −29.033 | 25.353 | 48.970 | 1.00 | 56.73 | A |
| 755 | O | ALA | A | 1107 | −30.090 | 25.933 | 48.908 | 1.00 | 56.16 | A |
| 756 | N | HIS | A | 1108 | −27.846 | 25.990 | 48.797 | 1.00 | 57.42 | A |
| 757 | CA | HIS | A | 1108 | −27.738 | 27.460 | 48.583 | 1.00 | 57.86 | A |
| 758 | CB | HIS | A | 1108 | −26.552 | 27.953 | 49.472 | 1.00 | 56.49 | A |
| 759 | CG | HIS | A | 1108 | −25.669 | 26.827 | 49.998 | 1.00 | 57.35 | A |
| 760 | CD2 | HIS | A | 1108 | −24.339 | 26.586 | 49.845 | 1.00 | 54.64 | A |
| 761 | ND1 | HIS | A | 1108 | −26.131 | 25.807 | 50.821 | 1.00 | 60.35 | A |
| 762 | CE1 | HIS | A | 1108 | −25.140 | 24.961 | 51.103 | 1.00 | 56.57 | A |
| 763 | NE2 | HIS | A | 1108 | −24.049 | 25.392 | 50.490 | 1.00 | 54.02 | A |
| 764 | C | HIS | A | 1108 | −27.758 | 28.058 | 46.986 | 1.00 | 59.34 | A |
| 765 | O | HIS | A | 1108 | −28.763 | 28.681 | 46.520 | 1.00 | 57.24 | A |
| 766 | N | ARG | A | 1109 | −26.698 | 27.907 | 46.179 | 1.00 | 58.79 | A |
| 767 | CA | ARG | A | 1109 | −26.754 | 28.555 | 44.894 | 1.00 | 59.92 | A |
| 768 | CB | ARG | A | 1109 | −27.415 | 29.876 | 44.981 | 1.00 | 59.48 | A |
| 769 | CG | RG | A | 1109 | −28.212 | 30.291 | 43.693 | 1.00 | 65.60 | A |
| 770 | CD | ARG | A | 1109 | −29.793 | 30.040 | 43.872 | 1.00 | 65.13 | A |
| 771 | NE | ARG | A | 1109 | −30.015 | 28.757 | 44.614 | 1.00 | 65.11 | A |
| 772 | CZ | ARG | A | 1109 | −31.172 | 28.349 | 45.173 | 1.00 | 61.19 | A |
| 773 | NH1 | ARG | A | 1109 | −32.316 | 29.022 | 45.071 | 1.00 | 66.22 | A |
| 774 | NH2 | ARG | A | 1109 | −31.212 | 27.243 | 45.803 | 1.00 | 50.65 | A |
| 775 | C | ARG | A | 1109 | −25.337 | 28.777 | 44.425 | 1.00 | 62.56 | A |
| 776 | O | ARG | A | 1109 | −25.059 | 29.348 | 43.326 | 1.00 | 61.66 | A |
| 777 | N | ARG | A | 1110 | −24.436 | 28.251 | 45.244 | 1.00 | 62.88 | A |
| 778 | CA | ARG | A | 1110 | −23.128 | 28.796 | 45.267 | 1.00 | 63.89 | A |
| 779 | CB | ARG | A | 1110 | −22.878 | 29.111 | 46.765 | 1.00 | 65.61 | A |
| 780 | CG | ARG | A | 1110 | −24.131 | 29.891 | 47.521 | 1.00 | 67.17 | A |
| 781 | CD | ARG | A | 1110 | −23.942 | 31.372 | 48.192 | 1.00 | 61.85 | A |
| 782 | NE | ARG | A | 1110 | −22.648 | 32.008 | 47.860 | 1.00 | 60.65 | A |
| 783 | CZ | ARG | A | 1110 | −22.375 | 33.323 | 48.025 | 1.00 | 60.78 | A |
| 784 | NH1 | ARG | A | 1110 | −23.336 | 34.143 | 48.475 | 1.00 | 56.21 | A |
| 785 | NH2 | ARG | A | 1110 | −21.176 | 33.838 | 47.671 | 1.00 | 55.22 | A |
| 786 | C | ARG | A | 1110 | −22.137 | 27.752 | 44.627 | 1.00 | 64.51 | A |
| 787 | O | ARG | A | 1110 | −22.293 | 26.533 | 44.796 | 1.00 | 66.52 | A |
| 788 | N | TYR | A | 1111 | −21.137 | 28.186 | 43.852 | 1.00 | 64.43 | A |
| 789 | CA | TYR | A | 1111 | −20.620 | 27.344 | 42.642 | 1.00 | 61.77 | A |
| 790 | CB | TYR | A | 1111 | −20.693 | 28.092 | 41.305 | 1.00 | 60.54 | A |
| 791 | CG | TYR | A | 1111 | −22.067 | 28.239 | 40.669 | 1.00 | 60.49 | A |
| 792 | CD1 | TYR | A | 1111 | −22.894 | 29.331 | 40.900 | 1.00 | 60.72 | A |
| 793 | CE1 | TYR | A | 1111 | −24.231 | 29.379 | 40.202 | 1.00 | 65.30 | A |
| 794 | CD2 | TYR | A | 1111 | −22.509 | 27.295 | 39.752 | 1.00 | 59.93 | A |
| 795 | CE2 | TYR | A | 1111 | −23.755 | 27.306 | 39.123 | 1.00 | 56.46 | A |
| 796 | CZ | TYR | A | 1111 | −24.610 | 28.312 | 39.320 | 1.00 | 64.19 | A |
| 797 | OH | TYR | A | 1111 | −25.809 | 28.214 | 38.620 | 1.00 | 64.45 | A |
| 798 | C | TYR | A | 1111 | −19.234 | 26.824 | 42.878 | 1.00 | 59.21 | A |
| 799 | O | TYR | A | 1111 | −18.492 | 27.456 | 43.620 | 1.00 | 58.87 | A |
| 800 | N | PHE | A | 1112 | −18.926 | 25.677 | 42.286 | 1.00 | 55.53 | A |
| 801 | CA | PHE | A | 1112 | −17.680 | 25.041 | 42.547 | 1.00 | 53.95 | A |
| 802 | CB | PHE | A | 1112 | −17.838 | 23.505 | 42.331 | 1.00 | 55.37 | A |
| 803 | CG | PHE | A | 1112 | −16.650 | 22.647 | 42.851 | 1.00 | 57.19 | A |
| 804 | CD1 | PHE | A | 1112 | −16.402 | 22.495 | 44.256 | 1.00 | 55.82 | A |
| 805 | CD2 | PHE | A | 1112 | −15.834 | 21.944 | 41.957 | 1.00 | 53.96 | A |
| 806 | CE1 | PHE | A | 1112 | −15.338 | 21.654 | 44.750 | 1.00 | 55.78 | A |
| 807 | CE2 | PHE | A | 1112 | −14.789 | 21.075 | 42.442 | 1.00 | 59.75 | A |
| 808 | CZ | PHE | A | 1112 | −14.543 | 20.925 | 43.877 | 1.00 | 56.33 | A |
| 809 | C | PHE | A | 1112 | −16.721 | 25.619 | 41.624 | 1.00 | 51.64 | A |
| 810 | O | PHE | A | 1112 | −17.122 | 25.849 | 40.522 | 1.00 | 53.63 | A |
| 811 | N | LY | A | 1113 | −15.452 | 25.792 | 42.021 | 1.00 | 50.07 | A |
| 812 | CA | GLY | A | 1113 | −14.379 | 26.364 | 41.198 | 1.00 | 46.39 | A |
| 813 | C | GLY | A | 1113 | −13.057 | 26.434 | 41.989 | 1.00 | 47.92 | A |
| 814 | O | GLY | A | 1113 | −12.917 | 25.822 | 43.019 | 1.00 | 48.18 | A |
| 815 | N | GLY | A | 1114 | −12.011 | 27.132 | 41.494 | 1.00 | 50.72 | A |
| 816 | CA | GLY | A | 1114 | −10.633 | 27.180 | 42.183 | 1.00 | 49.80 | A |
| 817 | C | GLY | A | 1114 | −9.479 | 26.725 | 41.368 | 1.00 | 52.50 | A |
| 818 | O | GLY | A | 1114 | −9.551 | 26.561 | 40.089 | 1.00 | 55.00 | A |
| 819 | N | THR | A | 1115 | −8.355 | 26.526 | 42.037 | 1.00 | 52.05 | A |
| 820 | CA | THR | A | 1115 | −7.364 | 25.556 | 41.509 | 1.00 | 50.12 | A |
| 821 | CB | THR | A | 1115 | −6.587 | 25.946 | 40.303 | 1.00 | 51.01 | A |
| 822 | OG1 | THR | A | 1115 | −5.528 | 24.902 | 40.101 | 1.00 | 56.05 | A |
| 823 | CG2 | THR | A | 1115 | −6.011 | 27.492 | 40.407 | 1.00 | 47.32 | A |
| 824 | C | THR | A | 1115 | −6.447 | 25.173 | 42.608 | 1.00 | 50.16 | A |
| 825 | O | THR | A | 1115 | −6.826 | 25.252 | 43.730 | 1.00 | 51.80 | A |
| 826 | N | GLU | A | 1116 | −5.301 | 24.636 | 42.293 | 1.00 | 49.91 | A |
| 827 | CA | GLU | A | 1116 | −4.563 | 23.746 | 43.174 | 1.00 | 51.63 | A |
| 828 | CB | GLU | A | 1116 | −3.179 | 24.458 | 43.488 | 1.00 | 54.50 | A |
| 829 | CG | GLU | A | 1116 | −1.860 | 23.673 | 43.060 | 1.00 | 59.58 | A |
| 830 | CD | GLU | A | 1116 | −1.660 | 23.532 | 41.469 | 1.00 | 69.91 | A |
| 831 | OE1 | GLU | A | 1116 | −1.168 | 22.430 | 41.020 | 1.00 | 62.25 | A |
| 832 | OE2 | GLU | A | 1116 | −2.076 | 24.504 | 40.683 | 1.00 | 73.04 | A |
| 833 | C | GLU | A | 1116 | −5.338 | 23.019 | 44.402 | 1.00 | 50.87 | A |
| 834 | O | GLU | A | 1116 | −6.482 | 22.600 | 44.325 | 1.00 | 50.01 | A |
| 835 | N | ASP | A | 1117 | −4.677 | 22.828 | 45.515 | 1.00 | 51.93 | A |
| 836 | CA | ASP | A | 1117 | −5.336 | 22.355 | 46.719 | 1.00 | 53.11 | A |
| 837 | CB | ASP | A | 1117 | −4.255 | 21.965 | 47.750 | 1.00 | 52.31 | A |
| 838 | CG | ASP | A | 1117 | −3.636 | 23.209 | 48.392 | 1.00 | 61.16 | A |
| 839 | OD1 | ASP | A | 1117 | −4.419 | 24.243 | 48.358 | 1.00 | 72.16 | A |
| 840 | OD2 | ASP | A | 1117 | −2.489 | 23.194 | 48.958 | 1.00 | 58.91 | A |
| 841 | C | ASP | A | 1117 | −6.108 | 23.568 | 47.178 | 1.00 | 51.47 | A |
| 842 | O | ASP | A | 1117 | −6.344 | 23.769 | 48.348 | 1.00 | 49.95 | A |
| 843 | N | ARG | A | 1118 | −6.420 | 24.420 | 46.254 | 1.00 | 51.96 | A |
| 844 | CA | ARG | A | 1118 | −7.143 | 25.627 | 46.653 | 1.00 | 54.21 | A |
| 845 | CB | ARG | A | 1118 | −6.306 | 26.857 | 46.406 | 1.00 | 53.19 | A |
| 846 | CG | ARG | A | 1118 | −6.983 | 28.233 | 46.695 | 1.00 | 56.03 | A |
| 847 | CD | ARG | A | 1118 | −6.768 | 28.869 | 48.164 | 1.00 | 51.44 | A |
| 848 | NE | ARG | A | 1118 | −7.847 | 28.342 | 48.939 | 1.00 | 54.38 | A |
| 849 | CZ | ARG | A | 1118 | −7.987 | 28.424 | 50.258 | 1.00 | 54.87 | A |
| 850 | NH1 | ARG | A | 1118 | −7.078 | 29.079 | 50.892 | 1.00 | 49.46 | A |
| 851 | NH2 | ARG | A | 1118 | −9.081 | 27.880 | 50.902 | 1.00 | 58.50 | A |
| 852 | C | ARG | A | 1118 | −8.565 | 25.766 | 45.997 | 1.00 | 54.93 | A |
| 853 | O | ARG | A | 1118 | −8.935 | 26.812 | 45.436 | 1.00 | 56.90 | A |
| 854 | N | LEU | A | 1119 | −9.328 | 24.689 | 46.059 | 1.00 | 52.65 | A |
| 855 | CA | LEU | A | 1119 | −10.565 | 24.634 | 45.469 | 1.00 | 51.53 | A |
| 856 | CB | LEU | A | 1119 | −10.727 | 23.173 | 45.089 | 1.00 | 50.26 | A |
| 857 | CG | LEU | A | 1119 | −11.264 | 23.096 | 43.669 | 1.00 | 53.50 | A |
| 858 | CD1 | LEU | A | 1119 | −10.517 | 23.536 | 42.356 | 1.00 | 49.79 | A |
| 859 | CD2 | LEU | A | 1119 | −11.660 | 21.723 | 43.575 | 1.00 | 58.40 | A |
| 860 | C | LEU | A | 1119 | −11.467 | 24.996 | 46.605 | 1.00 | 53.21 | A |
| 861 | O | LEU | A | 1119 | −11.105 | 24.794 | 47.828 | 1.00 | 56.04 | A |
| 862 | N | SER | A | 1120 | −12.683 | 25.399 | 46.235 | 1.00 | 52.68 | A |
| 863 | CA | SER | A | 1120 | −13.752 | 25.757 | 47.109 | 1.00 | 51.28 | A |
| 864 | CB | SER | A | 1120 | −13.504 | 27.116 | 47.796 | 1.00 | 53.56 | A |
| 865 | OG | SER | A | 1120 | −14.080 | 28.270 | 47.105 | 1.00 | 56.69 | A |
| 866 | C | SER | A | 1120 | −14.879 | 25.981 | 46.205 | 1.00 | 52.16 | A |
| 867 | O | SER | A | 1120 | −14.770 | 25.877 | 44.976 | 1.00 | 55.08 | A |
| 868 | N | CYS | A | 1121 | −15.909 | 26.524 | 46.790 | 1.00 | 51.98 | A |
| 869 | CA | CYS | A | 1121 | −17.169 | 26.590 | 46.205 | 1.00 | 53.07 | A |
| 870 | CB | CYS | A | 1121 | −17.821 | 25.112 | 46.167 | 1.00 | 50.71 | A |
| 871 | SG | CYS | A | 1121 | −19.698 | 24.993 | 45.537 | 1.00 | 56.99 | A |
| 872 | C | CYS | A | 1121 | −17.966 | 27.702 | 46.987 | 1.00 | 51.67 | A |
| 873 | O | CYS | A | 1121 | −19.205 | 27.676 | 47.019 | 1.00 | 52.16 | A |
| 874 | N | PHE | A | 1122 | −17.289 | 28.706 | 47.532 | 1.00 | 54.09 | A |
| 875 | CA | PHE | A | 1122 | −18.021 | 29.935 | 48.182 | 1.00 | 56.20 | A |
| 876 | CB | PHE | A | 1122 | −17.084 | 30.892 | 48.975 | 1.00 | 57.40 | A |
| 877 | CG | PHE | A | 1122 | −17.765 | 32.155 | 49.496 | 1.00 | 56.22 | A |
| 878 | CD1 | PHE | A | 1122 | −17.649 | 33.383 | 48.813 | 1.00 | 58.42 | A |
| 879 | CD2 | PHE | A | 1122 | −18.541 | 32.105 | 50.646 | 1.00 | 55.60 | A |
| 880 | CE1 | PHE | A | 1122 | −18.322 | 34.551 | 49.317 | 1.00 | 57.88 | A |
| 881 | CE2 | PHE | A | 1122 | −19.245 | 33.206 | 51.129 | 1.00 | 53.13 | A |
| 882 | CZ | PHE | A | 1122 | −19.154 | 34.440 | 50.444 | 1.00 | 58.16 | A |
| 883 | C | PHE | A | 1122 | −18.796 | 30.802 | 47.177 | 1.00 | 58.70 | A |
| 884 | O | PHE | A | 1122 | −19.692 | 31.631 | 47.562 | 1.00 | 58.90 | A |
| 885 | N | ALA | A | 1123 | −18.479 | 30.512 | 45.888 | 1.00 | 58.30 | A |
| 886 | CA | ALA | A | 1123 | −18.293 | 31.487 | 44.886 | 1.00 | 57.01 | A |
| 887 | CB | ALA | A | 1123 | −17.247 | 31.030 | 44.005 | 1.00 | 53.13 | A |
| 888 | C | ALA | A | 1123 | −19.609 | 31.418 | 44.210 | 1.00 | 59.39 | A |
| 889 | O | ALA | A | 1123 | −19.689 | 30.622 | 43.350 | 1.00 | 62.92 | A |
| 890 | N | GLN | A | 1124 | −20.592 | 32.248 | 44.565 | 1.00 | 58.59 | A |
| 891 | CA | GLN | A | 1124 | −21.892 | 32.263 | 44.036 | 1.00 | 60.33 | A |
| 892 | CB | GLN | A | 1124 | −22.797 | 33.004 | 45.023 | 1.00 | 61.66 | A |
| 893 | CG | GLN | A | 1124 | −22.699 | 34.627 | 45.106 | 1.00 | 60.20 | A |
| 894 | CD | GLN | A | 1124 | −24.007 | 35.186 | 45.928 | 1.00 | 65.71 | A |
| 895 | OE1 | GLN | A | 1124 | −24.051 | 36.374 | 46.385 | 1.00 | 74.45 | A |
| 896 | NE2 | GLN | A | 1124 | −25.051 | 34.322 | 46.103 | 1.00 | 56.96 | A |
| 897 | C | GLN | A | 1124 | −22.177 | 32.781 | 42.568 | 1.00 | 60.29 | A |
| 898 | O | GLN | A | 1124 | −23.411 | 33.090 | 42.248 | 1.00 | 57.22 | A |
| 899 | N | THR | A | 1125 | −21.127 | 32.779 | 41.696 | 1.00 | 59.56 | A |
| 900 | CA | THR | A | 1125 | −21.244 | 33.108 | 40.199 | 1.00 | 60.01 | A |
| 901 | CB | THR | A | 1125 | −21.101 | 34.645 | 39.943 | 1.00 | 60.67 | A |
| 902 | OG1 | THR | A | 1125 | −21.962 | 35.075 | 38.893 | 1.00 | 60.32 | A |
| 903 | CG2 | THR | A | 1125 | −19.612 | 34.942 | 39.517 | 1.00 | 57.52 | A |
| 904 | C | THR | A | 1125 | −20.113 | 32.420 | 39.260 | 1.00 | 61.10 | A |
| 905 | O | THR | A | 1125 | −19.204 | 31.750 | 39.763 | 1.00 | 62.25 | A |
| 906 | N | VAL | A | 1126 | −20.114 | 32.595 | 37.914 | 1.00 | 59.92 | A |
| 907 | CA | VAL | A | 1126 | −19.246 | 31.780 | 37.099 | 1.00 | 57.37 | A |
| 908 | CB | VAL | A | 1126 | −20.074 | 30.992 | 36.046 | 1.00 | 59.01 | A |
| 909 | CG1 | VAL | A | 1126 | −19.182 | 30.061 | 35.194 | 1.00 | 60.69 | A |
| 910 | CG2 | VAL | A | 1126 | −21.192 | 30.053 | 36.781 | 1.00 | 56.87 | A |
| 911 | C | VAL | A | 1126 | −18.049 | 32.523 | 36.642 | 1.00 | 56.73 | A |
| 912 | O | VAL | A | 1126 | −17.828 | 33.664 | 36.966 | 1.00 | 58.32 | A |
| 913 | N | SER | A | 1127 | −17.115 | 31.876 | 36.004 | 1.00 | 56.42 | A |
| 914 | CA | SER | A | 1127 | −15.795 | 32.474 | 35.981 | 1.00 | 53.96 | A |
| 915 | CB | SER | A | 1127 | −15.499 | 33.164 | 37.411 | 1.00 | 53.65 | A |
| 916 | OG | SER | A | 1127 | −14.142 | 33.645 | 37.645 | 1.00 | 53.32 | A |
| 917 | C | SER | A | 1127 | −14.912 | 31.249 | 35.587 | 1.00 | 52.67 | A |
| 918 | O | SER | A | 1127 | −15.029 | 30.261 | 36.195 | 1.00 | 48.36 | A |
| 919 | N | PRO | A | 1128 | −14.145 | 31.335 | 34.449 | 1.00 | 54.07 | A |
| 920 | CD | PRO | A | 1128 | −14.154 | 32.579 | 33.621 | 1.00 | 52.53 | A |
| 921 | CA | PRO | A | 1128 | −13.144 | 30.401 | 33.869 | 1.00 | 53.71 | A |
| 922 | CB | PRO | A | 1128 | −12.091 | 31.369 | 33.329 | 1.00 | 52.36 | A |
| 923 | CG | PRO | A | 1128 | −12.777 | 32.972 | 33.682 | 1.00 | 48.45 | A |
| 924 | C | PRO | A | 1128 | −12.442 | 29.503 | 34.925 | 1.00 | 55.63 | A |
| 925 | O | PRO | A | 1128 | −11.473 | 28.584 | 34.546 | 1.00 | 55.28 | A |
| 926 | N | ALA | A | 1129 | −12.891 | 29.792 | 36.189 | 1.00 | 52.77 | A |
| 927 | CA | ALA | A | 1129 | −12.588 | 28.993 | 37.414 | 1.00 | 52.53 | A |
| 928 | CB | ALA | A | 1129 | −11.748 | 29.794 | 38.421 | 1.00 | 52.73 | A |
| 929 | C | ALA | A | 1129 | −13.838 | 28.302 | 38.089 | 1.00 | 52.04 | A |
| 930 | O | ALA | A | 1129 | −13.673 | 27.535 | 38.949 | 1.00 | 51.07 | A |
| 931 | N | GLU | A | 1130 | −15.045 | 28.566 | 37.568 | 1.00 | 52.56 | A |
| 932 | CA | GLU | A | 1130 | −16.280 | 27.987 | 37.902 | 1.00 | 53.76 | A |
| 933 | CB | GLU | A | 1130 | −17.267 | 29.130 | 38.100 | 1.00 | 56.22 | A |
| 934 | CG | GLU | A | 1130 | −16.687 | 30.513 | 38.628 | 1.00 | 61.14 | A |
| 935 | CD | GLU | A | 1130 | −16.433 | 30.690 | 40.162 | 1.00 | 53.08 | A |
| 936 | OE1 | GLU | A | 1130 | −17.344 | 31.063 | 40.847 | 1.00 | 45.45 | A |
| 937 | OE2 | GLU | A | 1130 | −15.273 | 30.532 | 40.616 | 1.00 | 57.20 | A |
| 938 | C | GLU | A | 1130 | −16.880 | 27.055 | 36.788 | 1.00 | 53.52 | A |
| 939 | O | GLU | A | 1130 | −18.063 | 26.678 | 36.775 | 1.00 | 54.66 | A |
| 940 | N | LYS | A | 1131 | −16.051 | 26.732 | 35.816 | 1.00 | 52.78 | A |
| 941 | CA | LYS | A | 1131 | −16.470 | 26.225 | 34.535 | 1.00 | 49.88 | A |
| 942 | CB | LYS | A | 1131 | −16.093 | 27.290 | 33.506 | 1.00 | 47.21 | A |
| 943 | CG | LYS | A | 1131 | −17.057 | 27.591 | 32.218 | 1.00 | 51.45 | A |
| 944 | CD | LYS | A | 1131 | −18.589 | 28.266 | 32.338 | 1.00 | 43.76 | A |
| 945 | CE | LYS | A | 1131 | −18.836 | 29.070 | 31.014 | 1.00 | 48.44 | A |
| 946 | NZ | LYS | A | 1131 | −18.293 | 30.582 | 30.585 | 1.00 | 37.94 | A |
| 947 | C | LYS | A | 1131 | −15.587 | 24.957 | 34.452 | 1.00 | 49.00 | A |
| 948 | O | LYS | A | 1131 | −14.321 | 25.069 | 34.409 | 1.00 | 50.48 | A |
| 949 | N | TRP | A | 1132 | −16.200 | 23.792 | 34.555 | 1.00 | 47.26 | A |
| 950 | CA | TRP | A | 1132 | −15.462 | 22.542 | 34.417 | 1.00 | 50.44 | A |
| 951 | CB | TRP | A | 1132 | −15.808 | 21.648 | 35.568 | 1.00 | 50.76 | A |
| 952 | CG | TRP | A | 1132 | −15.496 | 22.268 | 36.968 | 1.00 | 50.73 | A |
| 953 | CD2 | TRP | A | 1132 | −14.317 | 22.059 | 37.722 | 1.00 | 44.90 | A |
| 954 | CE2 | TRP | A | 1132 | −14.451 | 22.786 | 38.904 | 1.00 | 48.50 | A |
| 955 | CE3 | TRP | A | 1132 | −13.165 | 21.306 | 37.504 | 1.00 | 48.73 | A |
| 956 | CD1 | TRP | A | 1132 | −16.329 | 23.076 | 37.751 | 1.00 | 48.97 | A |
| 957 | NE1 | TRP | A | 1132 | −15.690 | 23.406 | 38.886 | 1.00 | 46.71 | A |
| 958 | CZ2 | TRP | A | 1132 | −13.463 | 22.767 | 39.919 | 1.00 | 51.73 | A |
| 959 | CZ3 | TRP | A | 1132 | −12.206 | 21.298 | 38.439 | 1.00 | 52.00 | A |
| 960 | CH2 | TRP | A | 1132 | −12.353 | 22.045 | 39.683 | 1.00 | 52.93 | A |
| 961 | C | TRP | A | 1132 | −15.725 | 21.749 | 33.056 | 1.00 | 52.06 | A |
| 962 | O | TRP | A | 1132 | −16.912 | 21.697 | 32.549 | 1.00 | 51.71 | A |
| 963 | N | SER | A | 1133 | −14.653 | 21.191 | 32.449 | 1.00 | 51.83 | A |
| 964 | CA | SER | A | 1133 | −14.829 | 20.212 | 31.345 | 1.00 | 54.01 | A |
| 965 | CB | SER | A | 1133 | −13.844 | 20.428 | 30.159 | 1.00 | 53.50 | A |
| 966 | OG | SER | A | 1133 | −14.411 | 19.675 | 29.043 | 1.00 | 55.87 | A |
| 967 | C | SER | A | 1133 | −14.933 | 18.673 | 31.715 | 1.00 | 54.38 | A |
| 968 | O | SER | A | 1133 | −13.968 | 18.065 | 32.411 | 1.00 | 57.70 | A |
| 969 | N | VAL | A | 1134 | −16.004 | 18.037 | 31.242 | 1.00 | 52.77 | A |
| 970 | CA | VAL | A | 1134 | −16.173 | 16.604 | 31.383 | 1.00 | 55.10 | A |
| 971 | CB | VAL | A | 1134 | −17.510 | 16.098 | 30.897 | 1.00 | 56.03 | A |
| 972 | CG1 | VAL | A | 1134 | −18.117 | 15.118 | 31.965 | 1.00 | 51.56 | A |
| 973 | CG2 | VAL | A | 1134 | −18.452 | 17.284 | 30.462 | 1.00 | 60.83 | A |
| 974 | C | VAL | A | 1134 | −15.126 | 15.833 | 30.633 | 1.00 | 54.98 | A |
| 975 | O | VAL | A | 1134 | −14.668 | 16.324 | 29.679 | 1.00 | 59.19 | A |
| 976 | N | HIS | A | 1135 | −14.666 | 14.714 | 31.186 | 1.00 | 54.51 | A |
| 977 | CA | HIS | A | 1135 | −13.942 | 13.626 | 30.505 | 1.00 | 51.51 | A |
| 978 | CB | HIS | A | 1135 | −12.553 | 13.703 | 30.958 | 1.00 | 50.38 | A |
| 979 | CG | HIS | A | 1135 | −11.678 | 12.696 | 30.330 | 1.00 | 48.22 | A |
| 980 | CD2 | HIS | A | 1135 | −11.901 | 11.439 | 29.927 | 1.00 | 51.63 | A |
| 981 | ND1 | HIS | A | 1135 | −10.347 | 12.927 | 30.105 | 1.00 | 48.54 | A |
| 982 | CE1 | HIS | A | 1135 | −9.770 | 11.840 | 29.640 | 1.00 | 41.81 | A |
| 983 | NE2 | HIS | A | 1135 | −10.692 | 10.928 | 29.491 | 1.00 | 46.44 | A |
| 984 | C | HIS | A | 1135 | −14.530 | 12.208 | 30.953 | 1.00 | 48.59 | A |
| 985 | O | HIS | A | 1135 | −14.148 | 11.658 | 31.887 | 1.00 | 51.83 | A |
| 986 | N | ILE | A | 1136 | −15.536 | 11.710 | 30.332 | 1.00 | 44.99 | A |
| 987 | CA | ILE | A | 1136 | −16.240 | 10.558 | 30.705 | 1.00 | 42.87 | A |
| 988 | CB | ILE | A | 1136 | −17.179 | 10.350 | 29.530 | 1.00 | 43.16 | A |
| 989 | CG2 | ILE | A | 1136 | −16.795 | 9.275 | 28.721 | 1.00 | 45.12 | A |
| 990 | CG1 | ILE | A | 1136 | −18.618 | 10.358 | 29.975 | 1.00 | 46.38 | A |
| 991 | CD1 | ILE | A | 1136 | −19.272 | 11.691 | 29.533 | 1.00 | 50.17 | A |
| 992 | C | ILE | A | 1136 | −15.264 | 9.391 | 30.834 | 1.00 | 40.23 | A |
| 993 | O | ILE | A | 1136 | −14.241 | 9.422 | 30.248 | 1.00 | 39.58 | A |
| 994 | N | ALA | A | 1137 | −15.526 | 8.405 | 31.643 | 1.00 | 35.72 | A |
| 995 | CA | ALA | A | 1137 | −14.465 | 7.486 | 31.862 | 1.00 | 34.95 | A |
| 996 | CB | ALA | A | 1137 | −13.438 | 7.921 | 32.870 | 1.00 | 30.28 | A |
| 997 | C | ALA | A | 1137 | −15.178 | 6.131 | 32.247 | 1.00 | 39.24 | A |
| 998 | O | ALA | A | 1137 | −14.488 | 5.017 | 32.531 | 1.00 | 39.27 | A |
| 999 | N | MET | A | 1138 | −16.551 | 6.185 | 32.235 | 1.00 | 40.82 | A |
| 1000 | CA | MET | A | 1138 | −17.256 | 4.861 | 31.896 | 1.00 | 40.07 | A |
| 1001 | CB | MET | A | 1138 | −18.711 | 4.725 | 32.401 | 1.00 | 41.41 | A |
| 1002 | CG | MET | A | 1138 | −19.804 | 5.478 | 31.890 | 1.00 | 40.93 | A |
| 1003 | SD | MET | A | 1138 | −19.278 | 7.165 | 31.517 | 1.00 | 44.96 | A |
| 1004 | CE | MET | A | 1138 | −20.904 | 7.656 | 31.422 | 1.00 | 46.83 | A |
| 1005 | C | MET | A | 1138 | −17.155 | 4.426 | 30.518 | 1.00 | 41.23 | A |
| 1006 | O | MET | A | 1138 | −16.519 | 5.112 | 29.599 | 1.00 | 43.74 | A |
| 1007 | N | HIS | A | 1139 | −17.859 | 3.354 | 30.307 | 1.00 | 40.67 | A |
| 1008 | CA | HIS | A | 1139 | −17.865 | 2.594 | 28.956 | 1.00 | 42.79 | A |
| 1009 | CB | HIS | A | 1139 | −18.341 | 1.038 | 29.083 | 1.00 | 40.32 | A |
| 1010 | CG | HIS | A | 1139 | −17.948 | 0.251 | 27.890 | 1.00 | 39.28 | A |
| 1011 | CD2 | HIS | A | 1139 | −16.800 | −0.392 | 27.594 | 1.00 | 39.32 | A |
| 1012 | ND1 | HIS | A | 1139 | −18.718 | 0.222 | 26.732 | 1.00 | 40.16 | A |
| 1013 | CE1 | HIS | A | 1139 | −18.071 | −0.444 | 25.772 | 1.00 | 42.03 | A |
| 1014 | NE2 | HIS | A | 1139 | −16.901 | −0.825 | 26.279 | 1.00 | 52.16 | A |
| 1015 | C | HIS | A | 1139 | −18.652 | 3.457 | 27.885 | 1.00 | 42.45 | A |
| 1016 | O | HIS | A | 1139 | −19.715 | 4.072 | 28.125 | 1.00 | 44.34 | A |
| 1017 | N | PRO | A | 1140 | −18.096 | 3.669 | 26.753 | 1.00 | 40.56 | A |
| 1018 | CD | PRO | A | 1140 | −16.972 | 3.437 | 25.837 | 1.00 | 40.32 | A |
| 1019 | CA | PRO | A | 1140 | −19.052 | 4.628 | 26.224 | 1.00 | 39.82 | A |
| 1020 | CB | PRO | A | 1140 | −18.243 | 5.339 | 25.131 | 1.00 | 39.97 | A |
| 1021 | CG | PRO | A | 1140 | −17.028 | 4.636 | 24.925 | 1.00 | 38.75 | A |
| 1022 | C | PRO | A | 1140 | −20.274 | 4.021 | 25.466 | 1.00 | 41.62 | A |
| 1023 | O | PRO | A | 1140 | −21.085 | 4.864 | 24.754 | 1.00 | 44.31 | A |
| 1024 | N | GLN | A | 1141 | −20.464 | 2.665 | 25.512 | 1.00 | 38.73 | A |
| 1025 | CA | GLN | A | 1141 | −21.559 | 2.127 | 24.669 | 1.00 | 38.13 | A |
| 1026 | CB | GLN | A | 1141 | −21.110 | 0.979 | 23.740 | 1.00 | 37.92 | A |
| 1027 | CG | GLN | A | 1141 | −19.477 | 1.083 | 23.301 | 1.00 | 29.38 | A |
| 1028 | CD | GLN | A | 1141 | −19.232 | 0.057 | 22.227 | 1.00 | 35.36 | A |
| 1029 | OE1 | GLN | A | 1141 | −19.186 | −1.164 | 22.440 | 1.00 | 40.01 | A |
| 1030 | NE2 | GLN | A | 1141 | −19.351 | 0.498 | 21.061 | 1.00 | 39.51 | A |
| 1031 | C | GLN | A | 1141 | −22.752 | 1.892 | 25.566 | 1.00 | 39.01 | A |
| 1032 | O | GLN | A | 1141 | −22.547 | 1.615 | 26.663 | 1.00 | 42.93 | A |
| 1033 | N | VAL | A | 1142 | −24.000 | 2.216 | 25.146 | 1.00 | 40.22 | A |
| 1034 | CA | VAL | A | 1142 | −25.139 | 2.348 | 26.044 | 1.00 | 37.42 | A |
| 1035 | CB | VAL | A | 1142 | −25.190 | 3.804 | 26.607 | 1.00 | 36.84 | A |
| 1036 | CG1 | VAL | A | 1142 | −23.868 | 4.320 | 27.478 | 1.00 | 28.47 | A |
| 1037 | CG2 | VAL | A | 1142 | −25.542 | 4.844 | 25.513 | 1.00 | 32.12 | A |
| 1038 | C | VAL | A | 1142 | −26.407 | 1.917 | 25.169 | 1.00 | 41.45 | A |
| 1039 | O | VAL | A | 1142 | −26.376 | 1.731 | 23.873 | 1.00 | 38.54 | A |
| 1040 | N | ASN | A | 1143 | −27.493 | 1.699 | 25.917 | 1.00 | 42.60 | A |
| 1041 | CA | ASN | A | 1143 | −28.911 | 1.873 | 25.387 | 1.00 | 45.55 | A |
| 1042 | CB | ASN | A | 1143 | −29.742 | 0.680 | 25.767 | 1.00 | 43.97 | A |
| 1043 | CG | ASN | A | 1143 | −29.237 | −0.546 | 25.032 | 1.00 | 45.50 | A |
| 1044 | OD1 | ASN | A | 1143 | −29.357 | −1.664 | 25.549 | 1.00 | 40.76 | A |
| 1045 | ND2 | ASN | A | 1143 | −28.606 | −0.313 | 23.823 | 1.00 | 35.71 | A |
| 1046 | C | ASN | A | 1143 | −29.636 | 3.103 | 25.859 | 1.00 | 45.58 | A |
| 1047 | O | ASN | A | 1143 | −29.734 | 3.364 | 27.050 | 1.00 | 46.52 | A |
| 1048 | N | ILE | A | 1144 | −29.989 | 3.937 | 24.940 | 1.00 | 45.60 | A |
| 1049 | CA | ILE | A | 1144 | −30.633 | 5.128 | 25.377 | 1.00 | 46.25 | A |
| 1050 | CB | ILE | A | 1144 | −30.435 | 6.130 | 24.390 | 1.00 | 45.06 | A |
| 1051 | CG2 | ILE | A | 1144 | −31.608 | 6.974 | 24.427 | 1.00 | 39.54 | A |
| 1052 | CG1 | ILE | A | 1144 | −29.339 | 7.014 | 24.755 | 1.00 | 50.14 | A |
| 1053 | CD1 | ILE | A | 1144 | −28.081 | 7.006 | 23.929 | 1.00 | 61.13 | A |
| 1054 | C | ILE | A | 1144 | −32.175 | 4.942 | 25.368 | 1.00 | 48.07 | A |
| 1055 | O | ILE | A | 1144 | −32.754 | 4.582 | 24.322 | 1.00 | 49.53 | A |
| 1056 | N | TYR | A | 1145 | −32.816 | 5.217 | 26.513 | 1.00 | 48.69 | A |
| 1057 | CA | TYR | A | 1145 | −34.251 | 5.033 | 26.766 | 1.00 | 48.85 | A |
| 1058 | CB | TYR | A | 1145 | −34.367 | 4.256 | 27.958 | 1.00 | 48.27 | A |
| 1059 | CG | TYR | A | 1145 | −35.730 | 3.889 | 28.468 | 1.00 | 52.89 | A |
| 1060 | CD1 | TYR | A | 1145 | −36.494 | 3.030 | 27.712 | 1.00 | 57.74 | A |
| 1061 | CE1 | TYR | A | 1145 | −37.718 | 2.519 | 28.176 | 1.00 | 53.92 | A |
| 1062 | CD2 | TYR | A | 1145 | −36.165 | 4.154 | 29.832 | 1.00 | 49.36 | A |
| 1063 | CE2 | TYR | A | 1145 | −37.426 | 3.661 | 30.287 | 1.00 | 51.26 | A |
| 1064 | CZ | TYR | A | 1145 | −38.179 | 2.782 | 29.416 | 1.00 | 53.38 | A |
| 1065 | OH | TYR | A | 1145 | −39.443 | 2.143 | 29.593 | 1.00 | 52.73 | A |
| 1066 | C | TYR | A | 1145 | −34.792 | 6.431 | 27.059 | 1.00 | 50.45 | A |
| 1067 | O | TYR | A | 1145 | −33.998 | 7.409 | 27.213 | 1.00 | 52.93 | A |
| 1068 | N | SER | A | 1146 | −36.095 | 6.594 | 27.037 | 1.00 | 49.04 | A |
| 1069 | CA | SER | A | 1146 | −36.598 | 7.872 | 27.290 | 1.00 | 49.92 | A |
| 1070 | CB | SER | A | 1146 | −37.025 | 8.593 | 26.022 | 1.00 | 49.70 | A |
| 1071 | OG | SER | A | 1146 | −38.405 | 8.324 | 25.813 | 1.00 | 51.63 | A |
| 1072 | C | SER | A | 1146 | −37.830 | 7.685 | 28.170 | 1.00 | 52.46 | A |
| 1073 | O | SER | A | 1146 | −38.633 | 6.716 | 28.020 | 1.00 | 49.79 | A |
| 1074 | N | VAL | A | 1147 | −38.013 | 8.676 | 29.063 | 1.00 | 54.45 | A |
| 1075 | CA | VAL | A | 1147 | −39.024 | 8.601 | 30.075 | 1.00 | 55.75 | A |
| 1076 | CB | VAL | A | 1147 | −38.788 | 9.575 | 31.024 | 1.00 | 55.82 | A |
| 1077 | CG1 | VAL | A | 1147 | −39.326 | 9.086 | 32.319 | 1.00 | 62.77 | A |
| 1078 | CG2 | VAL | A | 1147 | −37.369 | 9.700 | 31.169 | 1.00 | 63.13 | A |
| 1079 | C | VAL | A | 1147 | −40.380 | 8.940 | 29.566 | 1.00 | 55.24 | A |
| 1080 | O | VAL | A | 1147 | −41.380 | 8.563 | 30.164 | 1.00 | 57.12 | A |
| 1081 | N | THR | A | 1148 | −40.433 | 9.642 | 28.465 | 1.00 | 55.75 | A |
| 1082 | CA | THR | A | 1148 | −41.698 | 10.258 | 27.987 | 1.00 | 55.72 | A |
| 1083 | CB | THR | A | 1148 | −41.373 | 11.452 | 27.035 | 1.00 | 55.61 | A |
| 1084 | OG1 | THR | A | 1148 | −40.383 | 12.325 | 27.670 | 1.00 | 52.66 | A |
| 1085 | CG2 | THR | A | 1148 | −42.661 | 12.203 | 26.574 | 1.00 | 51.68 | A |
| 1086 | C | THR | A | 1148 | −42.424 | 9.093 | 27.258 | 1.00 | 57.84 | A |
| 1087 | O | THR | A | 1148 | −43.344 | 8.402 | 27.866 | 1.00 | 56.21 | A |
| 1088 | N | ARG | A | 1149 | −41.977 | 8.897 | 25.991 | 1.00 | 56.85 | A |
| 1089 | CA | ARG | A | 1149 | −42.211 | 7.635 | 25.247 | 1.00 | 57.81 | A |
| 1090 | CB | ARG | A | 1149 | −41.430 | 7.591 | 23.925 | 1.00 | 57.12 | A |
| 1091 | CG | ARG | A | 1149 | −41.456 | 8.983 | 23.212 | 1.00 | 63.25 | A |
| 1092 | CD | ARG | A | 1149 | −43.014 | 9.356 | 22.546 | 1.00 | 69.05 | A |
| 1093 | NE | ARG | A | 1149 | −42.813 | 10.070 | 21.290 | 1.00 | 70.55 | A |
| 1094 | CZ | ARG | A | 1149 | −42.640 | 11.387 | 21.195 | 1.00 | 68.20 | A |
| 1095 | NH1 | ARG | A | 1149 | −42.830 | 12.109 | 22.335 | 1.00 | 60.99 | A |
| 1096 | NH2 | ARG | A | 1149 | −42.252 | 11.920 | 19.967 | 1.00 | 57.48 | A |
| 1097 | C | ARG | A | 1149 | −42.142 | 6.238 | 25.895 | 1.00 | 57.65 | A |
| 1098 | O | ARG | A | 1149 | −42.743 | 5.357 | 25.294 | 1.00 | 60.29 | A |
| 1099 | N | LYS | A | 1150 | −41.400 | 5.992 | 26.989 | 1.00 | 55.52 | A |
| 1100 | CA | LYS | A | 1150 | −41.265 | 4.598 | 27.551 | 1.00 | 53.30 | A |
| 1101 | CB | LYS | A | 1150 | −42.559 | 4.133 | 28.251 | 1.00 | 54.72 | A |
| 1102 | CG | LYS | A | 1150 | −42.723 | 4.806 | 29.745 | 1.00 | 49.24 | A |
| 1103 | CD | LYS | A | 1150 | −44.210 | 4.998 | 30.178 | 1.00 | 52.93 | A |
| 1104 | CE | LYS | A | 1150 | −44.464 | 4.112 | 31.466 | 1.00 | 51.43 | A |
| 1105 | NZ | LYS | A | 1150 | −43.693 | 2.842 | 31.275 | 1.00 | 53.85 | A |
| 1106 | C | LYS | A | 1150 | −40.681 | 3.534 | 26.673 | 1.00 | 52.03 | A |
| 1107 | O | LYS | A | 1150 | −41.005 | 2.346 | 26.807 | 1.00 | 50.14 | A |
| 1108 | N | ARG | A | 1151 | −39.734 | 3.998 | 25.811 | 1.00 | 53.34 | A |
| 1109 | CA | ARG | A | 1151 | −39.252 | 3.360 | 24.485 | 1.00 | 53.49 | A |
| 1110 | CB | ARG | A | 1151 | −39.997 | 3.873 | 23.213 | 1.00 | 51.42 | A |
| 1111 | CG | ARG | A | 1151 | −41.478 | 3.482 | 23.222 | 1.00 | 49.92 | A |
| 1112 | CD | ARG | A | 1151 | −41.595 | 1.862 | 23.598 | 1.00 | 32.45 | A |
| 1113 | NE | ARG | A | 1151 | −42.978 | 1.670 | 23.847 | 1.00 | 37.88 | A |
| 1114 | CZ | ARG | A | 1151 | −43.878 | 1.562 | 22.877 | 1.00 | 47.36 | A |
| 1115 | NH1 | ARG | A | 1151 | −43.430 | 1.565 | 21.567 | 1.00 | 38.19 | A |
| 1116 | NH2 | ARG | A | 1151 | −45.239 | 1.435 | 23.230 | 1.00 | 38.01 | A |
| 1117 | C | ARG | A | 1151 | −37.787 | 3.725 | 24.308 | 1.00 | 55.17 | A |
| 1118 | O | ARG | A | 1151 | −37.342 | 4.784 | 24.825 | 1.00 | 51.07 | A |
| 1119 | N | TYR | A | 1152 | −37.095 | 2.798 | 23.569 | 1.00 | 55.70 | A |
| 1120 | CA | TYR | A | 1152 | −35.698 | 2.776 | 23.282 | 1.00 | 54.64 | A |
| 1121 | CB | TYR | A | 1152 | −35.393 | 1.333 | 23.309 | 1.00 | 54.61 | A |
| 1122 | CG | TYR | A | 1152 | −35.141 | 0.900 | 24.708 | 1.00 | 58.49 | A |
| 1123 | CD1 | TYR | A | 1152 | −36.132 | 0.280 | 25.429 | 1.00 | 55.61 | A |
| 1124 | CE1 | TYR | A | 1152 | −35.914 | −0.121 | 26.688 | 1.00 | 49.92 | A |
| 1125 | CD2 | TYR | A | 1152 | −33.936 | 1.263 | 25.418 | 1.00 | 57.22 | A |
| 1126 | CE2 | TYR | A | 1152 | −33.770 | 0.827 | 26.798 | 1.00 | 47.70 | A |
| 1127 | CZ | TYR | A | 1152 | −34.748 | 0.156 | 27.353 | 1.00 | 47.04 | A |
| 1128 | OH | TYR | A | 1152 | −34.690 | −0.235 | 28.656 | 1.00 | 56.25 | A |
| 1129 | C | TYR | A | 1152 | −35.268 | 3.287 | 21.906 | 1.00 | 55.84 | A |
| 1130 | O | TYR | A | 1152 | −35.774 | 2.847 | 20.881 | 1.00 | 56.67 | A |
| 1131 | N | ALA | A | 1153 | −34.245 | 4.144 | 21.887 | 1.00 | 56.59 | A |
| 1132 | CA | ALA | A | 1153 | −33.760 | 4.832 | 20.732 | 1.00 | 55.99 | A |
| 1133 | CB | ALA | A | 1153 | −32.783 | 6.005 | 21.159 | 1.00 | 56.07 | A |
| 1134 | C | ALA | A | 1153 | −33.006 | 3.839 | 19.955 | 1.00 | 56.92 | A |
| 1135 | O | ALA | A | 1153 | −32.571 | 2.897 | 20.469 | 1.00 | 57.30 | A |
| 1136 | N | HIS | A | 1154 | −32.808 | 4.115 | 18.674 | 1.00 | 60.67 | A |
| 1137 | CA | HIS | A | 1154 | −31.980 | 3.237 | 17.781 | 1.00 | 61.19 | A |
| 1138 | CB | HIS | A | 1154 | −32.509 | 1.819 | 17.795 | 1.00 | 62.05 | A |
| 1139 | CG | HIS | A | 1154 | −33.888 | 1.769 | 17.278 | 1.00 | 64.88 | A |
| 1140 | CD2 | HIS | A | 1154 | −34.502 | 0.909 | 16.429 | 1.00 | 71.23 | A |
| 1141 | ND1 | HIS | A | 1154 | −34.810 | 2.719 | 17.639 | 1.00 | 67.70 | A |
| 1142 | CE1 | HIS | A | 1154 | −35.961 | 2.418 | 17.051 | 1.00 | 80.44 | A |
| 1143 | NE2 | HIS | A | 1154 | −35.798 | 1.329 | 16.301 | 1.00 | 78.35 | A |
| 1144 | C | HIS | A | 1154 | −32.241 | 3.637 | 16.353 | 1.00 | 58.30 | A |
| 1145 | O | HIS | A | 1154 | −33.111 | 4.457 | 16.139 | 1.00 | 55.10 | A |
| 1146 | N | LEU | A | 1155 | −31.563 | 2.900 | 15.439 | 1.00 | 57.17 | A |
| 1147 | CA | LEU | A | 1155 | −31.470 | 3.175 | 13.990 | 1.00 | 55.23 | A |
| 1148 | CB | LEU | A | 1155 | −30.212 | 2.553 | 13.423 | 1.00 | 55.99 | A |
| 1149 | CG | LEU | A | 1155 | −29.912 | 2.535 | 11.908 | 1.00 | 56.61 | A |
| 1150 | CD1 | LEU | A | 1155 | −29.517 | 4.092 | 11.605 | 1.00 | 51.06 | A |
| 1151 | CD2 | LEU | A | 1155 | −28.826 | 1.329 | 11.342 | 1.00 | 44.95 | A |
| 1152 | C | LEU | A | 1155 | −32.752 | 2.711 | 13.300 | 1.00 | 57.81 | A |
| 1153 | O | LEU | A | 1155 | −33.362 | 1.627 | 13.647 | 1.00 | 57.77 | A |
| 1154 | N | SER | A | 1156 | −33.195 | 3.610 | 12.409 | 1.00 | 58.62 | A |
| 1155 | CA | SER | A | 1156 | −34.488 | 3.599 | 11.720 | 1.00 | 57.48 | A |
| 1156 | CB | SER | A | 1156 | −34.700 | 4.997 | 11.079 | 1.00 | 57.42 | A |
| 1157 | OG | SER | A | 1156 | −36.082 | 5.267 | 10.670 | 1.00 | 56.33 | A |
| 1158 | C | SER | A | 1156 | −34.493 | 2.524 | 10.642 | 1.00 | 58.57 | A |
| 1159 | O | SER | A | 1156 | −33.451 | 1.907 | 10.362 | 1.00 | 56.39 | A |
| 1160 | N | ALA | A | 1157 | −35.650 | 2.430 | 9.941 | 1.00 | 62.16 | A |
| 1161 | CA | ALA | A | 1157 | −36.087 | 1.258 | 9.172 | 1.00 | 63.89 | A |
| 1162 | CB | ALA | A | 1157 | −37.416 | 0.797 | 9.731 | 1.00 | 63.50 | A |
| 1163 | C | ALA | A | 1157 | −36.150 | 1.504 | 7.630 | 1.00 | 66.42 | A |
| 1164 | O | ALA | A | 1157 | −35.105 | 1.555 | 6.904 | 1.00 | 66.46 | A |
| 1165 | N | ARG | A | 1158 | −37.377 | 1.652 | 7.130 | 1.00 | 67.87 | A |
| 1166 | CA | ARG | A | 1158 | −37.619 | 2.097 | 5.716 | 1.00 | 67.26 | A |
| 1167 | CB | ARG | A | 1158 | −39.040 | 1.720 | 5.228 | 1.00 | 66.92 | A |
| 1168 | CG | ARG | A | 1158 | −39.490 | 0.194 | 5.229 | 1.00 | 69.49 | A |
| 1169 | CD | ARG | A | 1158 | −38.660 | −0.806 | 4.374 | 1.00 | 70.89 | A |
| 1170 | NE | ARG | A | 1158 | −39.456 | −2.015 | 4.025 | 1.00 | 66.35 | A |
| 1171 | CZ | ARG | A | 1158 | −38.945 | −3.153 | 3.489 | 1.00 | 65.67 | A |
| 1172 | NH1 | ARG | A | 1158 | −37.641 | −3.309 | 3.203 | 1.00 | 62.24 | A |
| 1173 | NH2 | ARG | A | 1158 | −39.735 | −4.176 | 3.223 | 1.00 | 63.92 | A |
| 1174 | C | ARG | A | 1158 | −37.418 | 3.639 | 5.774 | 1.00 | 64.75 | A |
| 1175 | O | ARG | A | 1158 | −36.524 | 4.218 | 5.119 | 1.00 | 65.92 | A |
| 1176 | N | PRO | A | 1159 | −38.060 | 4.259 | 6.738 | 1.00 | 61.37 | A |
| 1177 | CD | PRO | A | 1159 | −38.882 | 3.757 | 7.818 | 1.00 | 61.45 | A |
| 1178 | CA | PRO | A | 1159 | −37.718 | 5.626 | 6.983 | 1.00 | 60.77 | A |
| 1179 | CB | PRO | A | 1159 | −38.681 | 5.997 | 8.114 | 1.00 | 60.75 | A |
| 1180 | CG | PRO | A | 1159 | −39.680 | 4.969 | 8.240 | 1.00 | 56.17 | A |
| 1181 | C | PRO | A | 1159 | −36.237 | 5.673 | 7.588 | 1.00 | 61.31 | A |
| 1182 | O | PRO | A | 1159 | −35.941 | 6.684 | 8.318 | 1.00 | 57.51 | A |
| 1183 | N | ALA | A | 1160 | −35.389 | 4.595 | 7.356 | 1.00 | 59.83 | A |
| 1184 | CA | ALA | A | 1160 | −33.987 | 4.635 | 7.826 | 1.00 | 60.22 | A |
| 1185 | CB | ALA | A | 1160 | −33.096 | 3.757 | 6.921 | 1.00 | 62.60 | A |
| 1186 | C | ALA | A | 1160 | −33.464 | 6.148 | 7.899 | 1.00 | 60.09 | A |
| 1187 | O | ALA | A | 1160 | −34.285 | 7.072 | 7.957 | 1.00 | 58.25 | A |
| 1188 | N | ASP | A | 1161 | −32.144 | 6.384 | 7.751 | 1.00 | 58.76 | A |
| 1189 | CA | ASP | A | 1161 | −31.508 | 7.701 | 7.975 | 1.00 | 57.33 | A |
| 1190 | CB | ASP | A | 1161 | −31.807 | 8.701 | 6.889 | 1.00 | 56.95 | A |
| 1191 | CG | ASP | A | 1161 | −30.681 | 9.841 | 6.809 | 1.00 | 60.13 | A |
| 1192 | OD1 | ASP | A | 1161 | −29.529 | 9.464 | 6.366 | 1.00 | 61.49 | A |
| 1193 | OD2 | ASP | A | 1161 | −30.929 | 11.047 | 7.250 | 1.00 | 57.19 | A |
| 1194 | C | ASP | A | 1161 | −31.746 | 8.412 | 9.339 | 1.00 | 55.49 | A |
| 1195 | O | ASP | A | 1161 | −31.079 | 9.356 | 9.686 | 1.00 | 54.97 | A |
| 1196 | N | GLU | A | 1162 | −32.723 | 7.984 | 10.090 | 1.00 | 55.55 | A |
| 1197 | CA | GLU | A | 1162 | −33.100 | 8.741 | 11.288 | 1.00 | 56.05 | A |
| 1198 | CB | GLU | A | 1162 | −34.553 | 9.233 | 11.191 | 1.00 | 53.04 | A |
| 1199 | CG | GLU | A | 1162 | −35.363 | 8.090 | 11.562 | 1.00 | 57.50 | A |
| 1200 | CD | GLU | A | 1162 | −36.885 | 8.199 | 11.483 | 1.00 | 60.84 | A |
| 1201 | OE1 | GLU | A | 1162 | −37.623 | 7.086 | 11.415 | 1.00 | 48.37 | A |
| 1202 | OE2 | GLU | A | 1162 | −37.283 | 9.392 | 11.523 | 1.00 | 61.98 | A |
| 1203 | C | GLU | A | 1162 | −32.894 | 7.638 | 12.425 | 1.00 | 56.30 | A |
| 1204 | O | GLU | A | 1162 | −33.069 | 6.345 | 12.132 | 1.00 | 54.69 | A |
| 1205 | N | ILE | A | 1163 | −32.488 | 8.118 | 13.635 | 1.00 | 53.87 | A |
| 1206 | CA | ILE | A | 1163 | −32.674 | 7.310 | 14.885 | 1.00 | 52.76 | A |
| 1207 | CB | ILE | A | 1163 | −31.704 | 7.727 | 16.054 | 1.00 | 53.12 | A |
| 1208 | CG2 | ILE | A | 1163 | −31.859 | 6.808 | 17.364 | 1.00 | 53.60 | A |
| 1209 | CG1 | ILE | A | 1163 | −30.259 | 7.554 | 15.603 | 1.00 | 57.71 | A |
| 1210 | CD1 | ILE | A | 1163 | −29.194 | 8.356 | 16.468 | 1.00 | 55.58 | A |
| 1211 | C | ILE | A | 1163 | −34.009 | 7.501 | 15.465 | 1.00 | 49.76 | A |
| 1212 | O | ILE | A | 1163 | −34.261 | 8.523 | 15.979 | 1.00 | 49.36 | A |
| 1213 | N | ALA | A | 1164 | −34.854 | 6.482 | 15.500 | 1.00 | 50.56 | A |
| 1214 | CA | ALA | A | 1164 | −36.054 | 6.544 | 16.330 | 1.00 | 50.15 | A |
| 1215 | CB | ALA | A | 1164 | −37.205 | 5.844 | 15.609 | 1.00 | 46.07 | A |
| 1216 | C | ALA | A | 1164 | −35.881 | 6.050 | 17.849 | 1.00 | 51.94 | A |
| 1217 | O | ALA | A | 1164 | −34.744 | 5.874 | 18.357 | 1.00 | 52.54 | A |
| 1218 | N | VAL | A | 1165 | −37.036 | 5.713 | 18.466 | 1.00 | 53.57 | A |
| 1219 | CA | VAL | A | 1165 | −37.253 | 5.301 | 19.768 | 1.00 | 53.73 | A |
| 1220 | CB | VAL | A | 1165 | −37.705 | 6.578 | 20.550 | 1.00 | 56.97 | A |
| 1221 | CG1 | VAL | A | 1165 | −36.488 | 7.685 | 21.122 | 1.00 | 51.57 | A |
| 1222 | CG2 | VAL | A | 1165 | −38.761 | 7.281 | 19.596 | 1.00 | 55.96 | A |
| 1223 | C | VAL | A | 1165 | −38.592 | 4.507 | 19.654 | 1.00 | 54.88 | A |
| 1224 | O | VAL | A | 1165 | −39.596 | 4.960 | 20.272 | 1.00 | 56.25 | A |
| 1225 | N | ASP | A | 1166 | −38.721 | 3.338 | 18.991 | 1.00 | 53.54 | A |
| 1226 | CA | ASP | A | 1166 | −40.016 | 2.702 | 19.183 | 1.00 | 51.08 | A |
| 1227 | CB | ASP | A | 1166 | −40.970 | 3.054 | 18.101 | 1.00 | 50.65 | A |
| 1228 | CG | ASP | A | 1166 | −40.262 | 3.252 | 16.714 | 1.00 | 56.08 | A |
| 1229 | OD1 | ASP | A | 1166 | −39.803 | 4.346 | 16.406 | 1.00 | 53.36 | A |
| 1230 | OD2 | ASP | A | 1166 | −40.033 | 2.246 | 15.919 | 1.00 | 71.03 | A |
| 1231 | C | ASP | A | 1166 | −39.918 | 1.222 | 19.378 | 1.00 | 55.17 | A |
| 1232 | O | ASP | A | 1166 | −40.338 | 0.404 | 18.366 | 1.00 | 54.94 | A |
| 1233 | N | ARG | A | 1167 | −39.442 | 0.828 | 20.643 | 1.00 | 53.75 | A |
| 1234 | CA | ARG | A | 1167 | −39.323 | −0.531 | 21.017 | 1.00 | 54.81 | A |
| 1235 | CB | ARG | A | 1167 | −38.137 | 1.115 | 20.228 | 1.00 | 56.32 | A |
| 1236 | CG | ARG | A | 1167 | −36.723 | −0.390 | 20.364 | 1.00 | 58.78 | A |
| 1237 | CD | ARG | A | 1167 | −35.681 | −0.506 | 19.040 | 1.00 | 56.26 | A |
| 1238 | NE | ARG | A | 1167 | −35.475 | −1.913 | 18.687 | 1.00 | 66.10 | A |
| 1239 | CZ | ARG | A | 1167 | −34.425 | −2.475 | 18.055 | 1.00 | 64.26 | A |
| 1240 | NH1 | ARG | A | 1167 | −33.380 | −1.791 | 17.648 | 1.00 | 67.66 | A |
| 1241 | NH2 | ARG | A | 1167 | −34.398 | −3.774 | 17.824 | 1.00 | 63.32 | A |
| 1242 | C | ARG | A | 1167 | −39.143 | −0.855 | 22.488 | 1.00 | 57.24 | A |
| 1243 | O | ARG | A | 1167 | −38.271 | −0.342 | 23.165 | 1.00 | 61.82 | A |
| 1244 | N | ASP | A | 1168 | −39.903 | −1.774 | 23.014 | 1.00 | 56.05 | A |
| 1245 | CA | ASP | A | 1168 | −40.062 | −1.945 | 24.388 | 1.00 | 53.81 | A |
| 1246 | CB | ASP | A | 1168 | −41.285 | −2.903 | 24.430 | 1.00 | 53.85 | A |
| 1247 | CG | ASP | A | 1168 | −42.126 | −2.857 | 25.745 | 1.00 | 57.19 | A |
| 1248 | OD1 | ASP | A | 1168 | −42.801 | −1.756 | 26.147 | 1.00 | 47.73 | A |
| 1249 | OD2 | ASP | A | 1168 | −42.122 | −4.031 | 26.301 | 1.00 | 55.70 | A |
| 1250 | C | ASP | A | 1168 | −38.814 | −2.700 | 24.818 | 1.00 | 56.17 | A |
| 1251 | O | ASP | A | 1168 | −38.709 | −3.252 | 26.003 | 1.00 | 60.36 | A |
| 1252 | N | VAL | A | 1169 | −37.879 | −2.937 | 23.908 | 1.00 | 53.28 | A |
| 1253 | CA | VAL | A | 1169 | −36.672 | −3.515 | 24.392 | 1.00 | 51.53 | A |
| 1254 | CB | VAL | A | 1169 | −36.813 | −4.995 | 24.750 | 1.00 | 51.11 | A |
| 1255 | CG1 | VAL | A | 1169 | −36.501 | −5.926 | 23.476 | 1.00 | 52.93 | A |
| 1256 | CG2 | VAL | A | 1169 | −35.893 | −5.410 | 26.028 | 1.00 | 47.17 | A |
| 1257 | C | VAL | A | 1169 | −35.762 | −3.172 | 23.292 | 1.00 | 53.59 | A |
| 1258 | O | VAL | A | 1169 | −36.141 | −3.222 | 22.146 | 1.00 | 57.16 | A |
| 1259 | N | PRO | A | 1170 | −34.558 | −2.753 | 23.592 | 1.00 | 53.16 | A |
| 1260 | CD | PRO | A | 1170 | −34.002 | −2.555 | 24.924 | 1.00 | 55.06 | A |
| 1261 | CA | PRO | A | 1170 | −33.574 | −2.548 | 22.565 | 1.00 | 50.23 | A |
| 1262 | CB | PRO | A | 1170 | −32.424 | −1.930 | 23.373 | 1.00 | 51.03 | A |
| 1263 | CG | PRO | A | 1170 | −32.495 | −2.584 | 24.678 | 1.00 | 51.68 | A |
| 1264 | C | PRO | A | 1170 | −32.991 | −3.852 | 21.997 | 1.00 | 50.54 | A |
| 1265 | O | PRO | A | 1170 | −31.966 | −4.328 | 22.595 | 1.00 | 51.10 | A |
| 1266 | N | TRP | A | 1171 | −33.529 | −4.459 | 20.900 | 1.00 | 45.96 | A |
| 1267 | CA | TRP | A | 1171 | −32.870 | −5.767 | 20.463 | 1.00 | 43.27 | A |
| 1268 | CB | TRP | A | 1171 | −33.932 | −6.850 | 20.228 | 1.00 | 43.79 | A |
| 1269 | CG | TRP | A | 1171 | −33.724 | −8.187 | 20.789 | 1.00 | 35.64 | A |
| 1270 | CD2 | TRP | A | 1171 | −33.597 | −8.511 | 22.178 | 1.00 | 31.83 | A |
| 1271 | CE2 | TRP | A | 1171 | −33.369 | −9.859 | 22.297 | 1.00 | 29.18 | A |
| 1272 | CE3 | TRP | A | 1171 | −33.823 | −7.775 | 23.371 | 1.00 | 43.31 | A |
| 1273 | CD1 | TRP | A | 1171 | −33.501 | −9.278 | 20.105 | 1.00 | 38.83 | A |
| 1274 | NE1 | TRP | A | 1171 | −33.141 | −10.331 | 21.056 | 1.00 | 40.85 | A |
| 1275 | CZ2 | TRP | A | 1171 | −33.258 | −10.498 | 23.555 | 1.00 | 44.74 | A |
| 1276 | CZ3 | TRP | A | 1171 | −33.765 | −8.433 | 24.695 | 1.00 | 38.91 | A |
| 1277 | CH2 | TRP | A | 1171 | −33.408 | −9.744 | 24.764 | 1.00 | 39.03 | A |
| 1278 | C | TRP | A | 1171 | −31.959 | −5.488 | 19.253 | 1.00 | 42.02 | A |
| 1279 | O | TRP | A | 1171 | −32.127 | −4.373 | 18.652 | 1.00 | 45.51 | A |
| 1280 | N | GLY | A | 1172 | −30.956 | −6.333 | 18.988 | 1.00 | 37.21 | A |
| 1281 | CA | GLY | A | 1172 | −29.812 | −6.174 | 18.123 | 1.00 | 36.44 | A |
| 1282 | C | GLY | A | 1172 | −29.299 | −4.772 | 17.737 | 1.00 | 42.21 | A |
| 1283 | O | GLY | A | 1172 | −29.577 | −3.745 | 18.389 | 1.00 | 44.18 | A |
| 1284 | N | VAL | A | 1173 | −28.594 | −4.694 | 16.582 | 1.00 | 45.05 | A |
| 1285 | CA | VAL | A | 1173 | −27.714 | −3.607 | 16.230 | 1.00 | 44.26 | A |
| 1286 | CB | VAL | A | 1173 | −26.944 | −3.782 | 14.904 | 1.00 | 43.11 | A |
| 1287 | CG1 | VAL | A | 1173 | −25.435 | −4.042 | 15.206 | 1.00 | 39.57 | A |
| 1288 | CG2 | VAL | A | 1173 | −27.614 | −4.542 | 13.733 | 1.00 | 33.62 | A |
| 1289 | C | VAL | A | 1173 | −28.261 | −2.187 | 16.097 | 1.00 | 47.76 | A |
| 1290 | O | VAL | A | 1173 | −27.537 | −1.241 | 15.702 | 1.00 | 51.06 | A |
| 1291 | N | ASP | A | 1174 | −29.515 | −1.998 | 16.332 | 1.00 | 47.47 | A |
| 1292 | CA | ASP | A | 1174 | −30.133 | −0.779 | 15.902 | 1.00 | 49.07 | A |
| 1293 | CB | ASP | A | 1174 | −31.574 | −1.150 | 15.516 | 1.00 | 47.81 | A |
| 1294 | CG | ASP | A | 1174 | −31.628 | −2.586 | 15.080 | 1.00 | 53.25 | A |
| 1295 | OD1 | ASP | A | 1174 | −30.935 | −2.924 | 14.062 | 1.00 | 62.21 | A |
| 1296 | OD2 | ASP | A | 1174 | −32.206 | −3.407 | 15.798 | 1.00 | 52.20 | A |
| 1297 | C | ASP | A | 1174 | −30.135 | −0.004 | 17.198 | 1.00 | 48.59 | A |
| 1298 | O | ASP | A | 1174 | −30.478 | 1.133 | 17.261 | 1.00 | 51.13 | A |
| 1299 | N | SER | A | 1175 | −29.852 | −0.688 | 18.260 | 1.00 | 45.38 | A |
| 1300 | CA | SER | A | 1175 | −30.133 | −0.188 | 19.460 | 1.00 | 43.01 | A |
| 1301 | CB | SER | A | 1175 | −30.520 | −1.373 | 20.232 | 1.00 | 44.33 | A |
| 1302 | OG | SER | A | 1175 | −31.937 | −1.451 | 20.312 | 1.00 | 46.67 | A |
| 1303 | C | SER | A | 1175 | −28.902 | 0.466 | 19.953 | 1.00 | 42.69 | A |
| 1304 | O | SER | A | 1175 | −29.040 | 1.445 | 20.561 | 1.00 | 47.61 | A |
| 1305 | N | LEU | A | 1176 | −27.703 | 0.085 | 19.576 | 1.00 | 43.34 | A |
| 1306 | CA | LEU | A | 1176 | −26.442 | 0.465 | 20.168 | 1.00 | 42.70 | A |
| 1307 | CB | LEU | A | 1176 | −25.389 | −0.608 | 19.891 | 1.00 | 44.56 | A |
| 1308 | CG | LEU | A | 1176 | −23.927 | −0.343 | 20.407 | 1.00 | 40.80 | A |
| 1309 | CD1 | LEU | A | 1176 | −23.785 | −0.914 | 21.682 | 1.00 | 28.04 | A |
| 1310 | CD2 | LEU | A | 1176 | −22.824 | −0.991 | 19.531 | 1.00 | 38.35 | A |
| 1311 | C | LEU | A | 1176 | −25.787 | 1.727 | 19.727 | 1.00 | 44.08 | A |
| 1312 | O | LEU | A | 1176 | −25.293 | 1.851 | 18.547 | 1.00 | 44.75 | A |
| 1313 | N | ILE | A | 1177 | −25.606 | 2.576 | 20.736 | 1.00 | 42.42 | A |
| 1314 | CA | ILE | A | 1177 | −25.150 | 3.921 | 20.587 | 1.00 | 41.76 | A |
| 1315 | CB | ILE | A | 1177 | −26.272 | 4.811 | 21.284 | 1.00 | 42.87 | A |
| 1316 | CG2 | ILE | A | 1177 | −25.818 | 6.326 | 21.517 | 1.00 | 41.23 | A |
| 1317 | CG1 | ILE | A | 1177 | −27.518 | 4.705 | 20.380 | 1.00 | 41.14 | A |
| 1318 | CD1 | ILE | A | 1177 | −28.729 | 5.595 | 20.689 | 1.00 | 41.52 | A |
| 1319 | C | ILE | A | 1177 | −23.799 | 4.168 | 21.234 | 1.00 | 42.23 | A |
| 1320 | O | ILE | A | 1177 | −23.518 | 3.608 | 22.280 | 1.00 | 47.69 | A |
| 1321 | N | THR | A | 1178 | −23.003 | 5.073 | 20.753 | 1.00 | 41.58 | A |
| 1322 | CA | THR | A | 1178 | −21.655 | 5.301 | 21.313 | 1.00 | 42.66 | A |
| 1323 | CB | THR | A | 1178 | −20.328 | 4.631 | 20.482 | 1.00 | 43.24 | A |
| 1324 | OG1 | THR | A | 1178 | −20.366 | 3.207 | 20.512 | 1.00 | 45.69 | A |
| 1325 | CG2 | THR | A | 1178 | −18.929 | 4.906 | 20.981 | 1.00 | 39.91 | A |
| 1326 | C | THR | A | 1178 | −21.531 | 6.774 | 21.629 | 1.00 | 45.89 | A |
| 1327 | O | THR | A | 1178 | −21.653 | 7.713 | 20.766 | 1.00 | 42.36 | A |
| 1328 | N | LEU | A | 1179 | −21.240 | 6.982 | 22.950 | 1.00 | 48.42 | A |
| 1329 | CA | LEU | A | 1179 | −21.004 | 8.294 | 23.365 | 1.00 | 48.14 | A |
| 1330 | CB | LEU | A | 1179 | −21.345 | 8.420 | 24.764 | 1.00 | 45.95 | A |
| 1331 | CG | LEU | A | 1179 | −22.819 | 8.159 | 25.043 | 1.00 | 47.99 | A |
| 1332 | CD1 | LEU | A | 1179 | −23.042 | 8.398 | 26.645 | 1.00 | 50.10 | A |
| 1333 | CD2 | LEU | A | 1179 | −23.847 | 9.013 | 24.322 | 1.00 | 36.60 | A |
| 1334 | C | LEU | A | 1179 | −19.555 | 8.477 | 23.028 | 1.00 | 50.76 | A |
| 1335 | O | LEU | A | 1179 | −18.663 | 8.406 | 23.895 | 1.00 | 54.63 | A |
| 1336 | N | ALA | A | 1180 | −19.277 | 8.705 | 21.739 | 1.00 | 51.61 | A |
| 1337 | CA | ALA | A | 1180 | −17.870 | 9.024 | 21.388 | 1.00 | 52.29 | A |
| 1338 | CB | ALA | A | 1180 | −17.609 | 8.850 | 19.960 | 1.00 | 51.30 | A |
| 1339 | C | ALA | A | 1180 | −17.449 | 10.476 | 21.825 | 1.00 | 51.43 | A |
| 1340 | O | ALA | A | 1180 | −18.245 | 11.278 | 22.107 | 1.00 | 48.12 | A |
| 1341 | N | PHE | A | 1181 | −16.151 | 10.718 | 21.884 | 1.00 | 54.36 | A |
| 1342 | CA | PHE | A | 1181 | −15.582 | 12.021 | 22.014 | 1.00 | 55.65 | A |
| 1343 | CB | PHE | A | 1181 | −14.106 | 11.896 | 22.295 | 1.00 | 55.80 | A |
| 1344 | CG | PHE | A | 1181 | −13.740 | 11.072 | 23.434 | 1.00 | 52.74 | A |
| 1345 | CD1 | PHE | A | 1181 | −14.096 | 11.447 | 24.725 | 1.00 | 53.31 | A |
| 1346 | CD2 | PHE | A | 1181 | −12.856 | 10.013 | 23.264 | 1.00 | 54.49 | A |
| 1347 | CE1 | PHE | A | 1181 | −13.609 | 10.759 | 25.919 | 1.00 | 49.29 | A |
| 1348 | CE2 | PHE | A | 1181 | −12.393 | 9.206 | 24.445 | 1.00 | 43.68 | A |
| 1349 | CZ | PHE | A | 1181 | −12.810 | 9.620 | 25.734 | 1.00 | 55.56 | A |
| 1350 | C | PHE | A | 1181 | −15.619 | 12.807 | 20.730 | 1.00 | 58.19 | A |
| 1351 | O | PHE | A | 1181 | −15.921 | 12.342 | 19.589 | 1.00 | 59.27 | A |
| 1352 | N | GLN | A | 1182 | −15.119 | 13.992 | 20.948 | 1.00 | 59.83 | A |
| 1353 | CA | GLN | A | 1182 | −15.404 | 15.188 | 20.177 | 1.00 | 63.20 | A |
| 1354 | CB | GLN | A | 1182 | −16.958 | 15.520 | 19.992 | 1.00 | 60.39 | A |
| 1355 | CG | GLN | A | 1182 | −17.263 | 15.527 | 18.434 | 1.00 | 68.90 | A |
| 1356 | CD | GLN | A | 1182 | −18.416 | 16.452 | 17.773 | 1.00 | 69.42 | A |
| 1357 | OE1 | GLN | A | 1182 | −18.719 | 17.578 | 18.210 | 1.00 | 81.45 | A |
| 1358 | NE2 | GLN | A | 1182 | −19.011 | 15.946 | 16.716 | 1.00 | 71.57 | A |
| 1359 | C | GLN | A | 1182 | −14.558 | 16.263 | 20.959 | 1.00 | 61.62 | A |
| 1360 | O | GLN | A | 1182 | −13.406 | 16.035 | 21.392 | 1.00 | 61.76 | A |
| 1361 | N | ASP | A | 1183 | −15.083 | 17.430 | 21.139 | 1.00 | 60.93 | A |
| 1362 | CA | ASP | A | 1183 | −14.176 | 18.413 | 21.586 | 1.00 | 62.18 | A |
| 1363 | CB | ASP | A | 1183 | −13.604 | 19.312 | 20.452 | 1.00 | 61.58 | A |
| 1364 | CG | ASP | A | 1183 | −14.671 | 19.682 | 19.333 | 1.00 | 63.90 | A |
| 1365 | OD1 | ASP | A | 1183 | −15.793 | 20.241 | 19.690 | 1.00 | 61.47 | A |
| 1366 | OD2 | ASP | A | 1183 | −14.336 | 19.418 | 18.099 | 1.00 | 61.93 | A |
| 1367 | C | ASP | A | 1183 | −14.970 | 19.078 | 22.683 | 1.00 | 62.92 | A |
| 1368 | O | ASP | A | 1183 | −15.812 | 20.014 | 22.443 | 1.00 | 62.62 | A |
| 1369 | N | GLN | A | 1184 | −14.742 | 18.456 | 23.876 | 1.00 | 60.93 | A |
| 1370 | CA | GLN | A | 1184 | −15.360 | 18.890 | 25.112 | 1.00 | 59.39 | A |
| 1371 | CB | GLN | A | 1184 | −15.045 | 20.419 | 25.457 | 1.00 | 58.37 | A |
| 1372 | CG | GLN | A | 1184 | −13.422 | 20.721 | 25.438 | 1.00 | 59.71 | A |
| 1373 | CD | GLN | A | 1184 | −12.484 | 19.349 | 25.450 | 1.00 | 63.12 | A |
| 1374 | OE1 | GLN | A | 1184 | −12.634 | 18.412 | 26.340 | 1.00 | 68.29 | A |
| 1375 | NE2 | GLN | A | 1184 | −11.539 | 19.255 | 24.488 | 1.00 | 51.48 | A |
| 1376 | C | GLN | A | 1184 | −16.741 | 18.565 | 24.709 | 1.00 | 58.21 | A |
| 1377 | O | GLN | A | 1184 | −17.747 | 19.225 | 24.980 | 1.00 | 60.69 | A |
| 1378 | N | ARG | A | 1185 | −16.835 | 17.545 | 23.945 | 1.00 | 55.63 | A |
| 1379 | CA | ARG | A | 1185 | −18.206 | 17.350 | 23.532 | 1.00 | 54.81 | A |
| 1380 | CB | ARG | A | 1185 | −18.549 | 18.370 | 22.438 | 1.00 | 53.90 | A |
| 1381 | CG | ARG | A | 1185 | −19.202 | 19.725 | 22.998 | 1.00 | 56.94 | A |
| 1382 | CD | ARG | A | 1185 | −20.234 | 20.450 | 21.838 | 1.00 | 55.54 | A |
| 1383 | NE | ARG | A | 1185 | −20.678 | 21.761 | 22.186 | 1.00 | 52.09 | A |
| 1384 | CZ | ARG | A | 1185 | −21.757 | 22.000 | 22.949 | 1.00 | 55.77 | A |
| 1385 | NH1 | ARG | A | 1185 | −22.597 | 21.005 | 23.277 | 1.00 | 55.38 | A |
| 1386 | NH2 | ARG | A | 1185 | −22.087 | 23.281 | 23.295 | 1.00 | 52.30 | A |
| 1387 | C | ARG | A | 1185 | −18.491 | 15.868 | 23.198 | 1.00 | 52.14 | A |
| 1388 | O | ARG | A | 1185 | −17.543 | 15.060 | 23.014 | 1.00 | 47.69 | A |
| 1389 | N | TYR | A | 1186 | −19.767 | 15.490 | 23.126 | 1.00 | 49.53 | A |
| 1390 | CA | TYR | A | 1186 | −19.987 | 14.031 | 22.848 | 1.00 | 48.90 | A |
| 1391 | CB | TYR | A | 1186 | −20.145 | 13.344 | 24.188 | 1.00 | 49.15 | A |
| 1392 | CG | TYR | A | 1186 | −18.960 | 13.360 | 25.180 | 1.00 | 43.95 | A |
| 1393 | CD1 | TYR | A | 1186 | −17.991 | 12.478 | 25.050 | 1.00 | 38.49 | A |
| 1394 | CE1 | TYR | A | 1186 | −16.933 | 12.363 | 25.867 | 1.00 | 45.67 | A |
| 1395 | CD2 | TYR | A | 1186 | −18.933 | 14.163 | 26.272 | 1.00 | 46.78 | A |
| 1396 | CE2 | TYR | A | 1186 | −17.857 | 14.048 | 27.243 | 1.00 | 53.74 | A |
| 1397 | CZ | TYR | A | 1186 | −16.853 | 13.113 | 26.976 | 1.00 | 52.64 | A |
| 1398 | OH | TYR | A | 1186 | −15.789 | 12.923 | 27.730 | 1.00 | 49.85 | A |
| 1399 | C | TYR | A | 1186 | −21.167 | 13.638 | 21.885 | 1.00 | 51.68 | A |
| 1400 | O | TYR | A | 1186 | −22.415 | 13.576 | 22.295 | 1.00 | 53.77 | A |
| 1401 | N | SER | A | 1187 | −20.874 | 13.370 | 20.607 | 1.00 | 51.07 | A |
| 1402 | CA | SER | A | 1187 | −21.949 | 12.821 | 19.698 | 1.00 | 50.53 | A |
| 1403 | CB | SER | A | 1187 | −21.509 | 12.842 | 18.256 | 1.00 | 50.97 | A |
| 1404 | OG | SER | A | 1187 | −20.240 | 12.399 | 18.196 | 1.00 | 46.43 | A |
| 1405 | C | SER | A | 1187 | −22.468 | 11.420 | 19.902 | 1.00 | 50.50 | A |
| 1406 | O | SER | A | 1187 | −21.768 | 10.518 | 20.051 | 1.00 | 53.19 | A |
| 1407 | N | VAL | A | 1188 | −23.730 | 11.244 | 19.900 | 1.00 | 49.79 | A |
| 1408 | CA | VAL | A | 1188 | −24.264 | 9.950 | 19.660 | 1.00 | 50.91 | A |
| 1409 | CB | VAL | A | 1188 | −25.768 | 10.067 | 19.982 | 1.00 | 53.23 | A |
| 1410 | CG1 | VAL | A | 1188 | −25.910 | 10.318 | 21.545 | 1.00 | 47.80 | A |
| 1411 | CG2 | VAL | A | 1188 | −26.384 | 11.324 | 19.273 | 1.00 | 54.19 | A |
| 1412 | C | VAL | A | 1188 | −23.983 | 9.255 | 18.287 | 1.00 | 51.02 | A |
| 1413 | O | VAL | A | 1188 | −24.601 | 9.469 | 17.215 | 1.00 | 52.30 | A |
| 1414 | N | GLN | A | 1189 | −23.031 | 8.381 | 18.340 | 1.00 | 51.11 | A |
| 1415 | CA | GLN | A | 1189 | −22.589 | 7.730 | 17.164 | 1.00 | 51.01 | A |
| 1416 | CB | GLN | A | 1189 | −21.104 | 7.314 | 17.179 | 1.00 | 50.24 | A |
| 1417 | CG | GLN | A | 1189 | −20.700 | 6.676 | 15.832 | 1.00 | 40.12 | A |
| 1418 | CD | GLN | A | 1189 | −19.217 | 6.617 | 15.776 | 1.00 | 48.75 | A |
| 1419 | OE1 | GLN | A | 1189 | −18.633 | 6.927 | 14.663 | 1.00 | 41.63 | A |
| 1420 | NE2 | GLN | A | 1189 | −18.500 | 6.314 | 17.055 | 1.00 | 39.82 | A |
| 1421 | C | GLN | A | 1189 | −23.253 | 6.430 | 17.172 | 1.00 | 51.60 | A |
| 1422 | O | GLN | A | 1189 | −23.268 | 5.773 | 18.202 | 1.00 | 52.55 | A |
| 1423 | N | THR | A | 1190 | −23.568 | 5.990 | 15.947 | 1.00 | 49.64 | A |
| 1424 | CA | THR | A | 1190 | −24.546 | 4.967 | 15.749 | 1.00 | 45.96 | A |
| 1425 | CB | THR | A | 1190 | −25.375 | 5.475 | 14.541 | 1.00 | 44.37 | A |
| 1426 | OG1 | THR | A | 1190 | −24.538 | 6.391 | 13.854 | 1.00 | 43.61 | A |
| 1427 | CG2 | THR | A | 1190 | −26.390 | 6.228 | 15.074 | 1.00 | 45.56 | A |
| 1428 | C | THR | A | 1190 | −23.787 | 3.693 | 15.497 | 1.00 | 41.56 | A |
| 1429 | O | THR | A | 1190 | −22.656 | 3.815 | 15.348 | 1.00 | 38.29 | A |
| 1430 | N | ALA | A | 1191 | −24.430 | 2.555 | 15.246 | 1.00 | 42.07 | A |
| 1431 | CA | ALA | A | 1191 | −23.701 | 1.286 | 14.972 | 1.00 | 45.40 | A |
| 1432 | CB | ALA | A | 1191 | −24.487 | 0.021 | 15.338 | 1.00 | 43.33 | A |
| 1433 | C | ALA | A | 1191 | −23.098 | 1.100 | 13.614 | 1.00 | 47.54 | A |
| 1434 | O | ALA | A | 1191 | −22.289 | 0.169 | 13.501 | 1.00 | 48.25 | A |
| 1435 | N | ASP | A | 1192 | −23.510 | 1.919 | 12.639 | 1.00 | 48.63 | A |
| 1436 | CA | ASP | A | 1192 | −22.907 | 1.988 | 11.308 | 1.00 | 49.53 | A |
| 1437 | CB | ASP | A | 1192 | −23.995 | 2.171 | 10.258 | 1.00 | 52.00 | A |
| 1438 | CG | ASP | A | 1192 | −24.994 | 3.411 | 10.562 | 1.00 | 56.24 | A |
| 1439 | OD1 | ASP | A | 1192 | −24.748 | 4.155 | 11.561 | 1.00 | 60.73 | A |
| 1440 | OD2 | ASP | A | 1192 | −25.978 | 3.619 | 9.776 | 1.00 | 52.99 | A |
| 1441 | C | ASP | A | 1192 | −21.920 | 3.141 | 11.136 | 1.00 | 49.08 | A |
| 1442 | O | ASP | A | 1192 | −21.324 | 3.325 | 10.039 | 1.00 | 49.27 | A |
| 1443 | N | HIS | A | 1193 | −21.685 | 3.886 | 12.195 | 1.00 | 48.30 | A |
| 1444 | CA | HIS | A | 1193 | −20.533 | 4.838 | 12.314 | 1.00 | 47.88 | A |
| 1445 | CB | HIS | A | 1193 | −19.599 | 4.636 | 11.211 | 1.00 | 48.10 | A |
| 1446 | CG | HIS | A | 1193 | −18.775 | 3.443 | 11.386 | 1.00 | 48.11 | A |
| 1447 | CD2 | HIS | A | 1193 | −17.577 | 3.111 | 10.915 | 1.00 | 46.17 | A |
| 1448 | ND1 | HIS | A | 1193 | −19.156 | 2.421 | 12.207 | 1.00 | 56.60 | A |
| 1449 | CE1 | HIS | A | 1193 | −18.258 | 1.462 | 12.161 | 1.00 | 49.47 | A |
| 1450 | NE2 | HIS | A | 1193 | −17.266 | 1.893 | 11.441 | 1.00 | 46.34 | A |
| 1451 | C | HIS | A | 1193 | −20.927 | 6.311 | 12.233 | 1.00 | 49.66 | A |
| 1452 | O | HIS | A | 1193 | −20.086 | 7.236 | 12.346 | 1.00 | 52.17 | A |
| 1453 | N | ARG | A | 1194 | −22.183 | 6.573 | 12.041 | 1.00 | 49.16 | A |
| 1454 | CA | ARG | A | 1194 | −22.456 | 7.896 | 11.699 | 1.00 | 50.30 | A |
| 1455 | CB | ARG | A | 1194 | −23.584 | 7.971 | 10.625 | 1.00 | 54.54 | A |
| 1456 | CG | ARG | A | 1194 | −23.608 | 7.074 | 9.301 | 1.00 | 49.25 | A |
| 1457 | CD | ARG | A | 1194 | −24.944 | 7.273 | 8.725 | 1.00 | 45.22 | A |
| 1458 | NE | ARG | A | 1194 | −25.729 | 6.082 | 8.994 | 1.00 | 50.21 | A |
| 1459 | CZ | ARG | A | 1194 | −26.957 | 5.903 | 8.472 | 1.00 | 54.72 | A |
| 1460 | NH1 | ARG | A | 1194 | −27.474 | 6.917 | 7.787 | 1.00 | 55.93 | A |
| 1461 | NH2 | ARG | A | 1194 | −27.683 | 4.764 | 8.621 | 1.00 | 52.24 | A |
| 1462 | C | ARG | A | 1194 | −23.028 | 8.533 | 12.965 | 1.00 | 51.72 | A |
| 1463 | O | ARG | A | 1194 | −23.289 | 7.858 | 13.971 | 1.00 | 53.54 | A |
| 1464 | N | PHE | A | 1195 | −23.261 | 9.831 | 12.893 | 1.00 | 51.55 | A |
| 1465 | CA | PHE | A | 1195 | −23.519 | 10.617 | 14.081 | 1.00 | 51.97 | A |
| 1466 | CB | PHE | A | 1195 | −22.474 | 11.676 | 14.373 | 1.00 | 47.85 | A |
| 1467 | CG | PHE | A | 1195 | −21.013 | 11.177 | 14.338 | 1.00 | 47.97 | A |
| 1468 | CD1 | PHE | A | 1195 | −20.260 | 11.207 | 13.125 | 1.00 | 45.55 | A |
| 1469 | CD2 | PHE | A | 1195 | −20.339 | 10.826 | 15.539 | 1.00 | 40.06 | A |
| 1470 | CE1 | PHE | A | 1195 | −18.879 | 10.886 | 13.141 | 1.00 | 51.26 | A |
| 1471 | CE2 | PHE | A | 1195 | −18.986 | 10.415 | 15.563 | 1.00 | 44.02 | A |
| 1472 | CZ | PHE | A | 1195 | −18.222 | 10.401 | 14.389 | 1.00 | 43.72 | A |
| 1473 | C | PHE | A | 1195 | −24.951 | 11.170 | 13.993 | 1.00 | 53.19 | A |
| 1474 | O | PHE | A | 1195 | −25.619 | 10.990 | 12.947 | 1.00 | 51.90 | A |
| 1475 | N | LEU | A | 1196 | −25.472 | 11.716 | 15.120 | 1.00 | 53.01 | A |
| 1476 | CA | LEU | A | 1196 | −26.740 | 12.504 | 14.967 | 1.00 | 54.25 | A |
| 1477 | CB | LEU | A | 1196 | −27.686 | 12.081 | 16.075 | 1.00 | 53.91 | A |
| 1478 | CG | LEU | A | 1196 | −29.158 | 12.444 | 16.241 | 1.00 | 51.72 | A |
| 1479 | CD1 | LEU | A | 1196 | −30.107 | 11.568 | 15.518 | 1.00 | 46.79 | A |
| 1480 | CD2 | LEU | A | 1196 | −29.485 | 12.313 | 17.653 | 1.00 | 52.55 | A |
| 1481 | C | LEU | A | 1196 | −26.646 | 14.089 | 14.786 | 1.00 | 54.79 | A |
| 1482 | O | LEU | A | 1196 | −26.526 | 14.815 | 15.694 | 1.00 | 55.60 | A |
| 1483 | N | ARG | A | 1197 | −26.674 | 14.653 | 13.620 | 1.00 | 57.25 | A |
| 1484 | CA | ARG | A | 1197 | −26.525 | 16.093 | 13.649 | 1.00 | 59.59 | A |
| 1485 | CB | ARG | A | 1197 | −26.770 | 16.698 | 12.236 | 1.00 | 61.90 | A |
| 1486 | CG | ARG | A | 1197 | −26.635 | 18.274 | 12.041 | 1.00 | 60.84 | A |
| 1487 | CD | ARG | A | 1197 | −26.532 | 18.411 | 10.551 | 1.00 | 61.17 | A |
| 1488 | NE | ARG | A | 1197 | −25.521 | 17.447 | 10.129 | 1.00 | 71.05 | A |
| 1489 | CZ | ARG | A | 1197 | −24.355 | 17.727 | 9.521 | 1.00 | 71.31 | A |
| 1490 | NH1 | ARG | A | 1197 | −24.050 | 18.970 | 9.183 | 1.00 | 71.86 | A |
| 1491 | NH2 | ARG | A | 1197 | −23.495 | 16.746 | 9.220 | 1.00 | 72.37 | A |
| 1492 | C | ARG | A | 1197 | −27.574 | 16.639 | 14.601 | 1.00 | 59.05 | A |
| 1493 | O | ARG | A | 1197 | −28.801 | 16.248 | 14.608 | 1.00 | 60.61 | A |
| 1494 | N | HIS | A | 1198 | −27.103 | 17.496 | 15.461 | 1.00 | 57.90 | A |
| 1495 | CA | HIS | A | 1198 | −28.062 | 18.256 | 16.292 | 1.00 | 57.50 | A |
| 1496 | CB | HIS | A | 1198 | −27.381 | 19.519 | 16.848 | 1.00 | 57.78 | A |
| 1497 | CG | HIS | A | 1198 | −27.875 | 20.801 | 16.299 | 1.00 | 58.21 | A |
| 1498 | CD2 | HIS | A | 1198 | −29.123 | 21.327 | 16.204 | 1.00 | 57.81 | A |
| 1499 | ND1 | HIS | A | 1198 | −27.009 | 21.750 | 15.793 | 1.00 | 61.93 | A |
| 1500 | CE1 | HIS | A | 1198 | −27.707 | 22.815 | 15.425 | 1.00 | 56.68 | A |
| 1501 | NE2 | HIS | A | 1198 | −28.986 | 22.579 | 15.677 | 1.00 | 56.90 | A |
| 1502 | C | HIS | A | 1198 | −29.373 | 18.464 | 15.521 | 1.00 | 55.84 | A |
| 1503 | O | HIS | A | 1198 | −30.474 | 18.275 | 16.097 | 1.00 | 55.29 | A |
| 1504 | N | ASP | A | 1199 | −29.250 | 18.621 | 14.198 | 1.00 | 55.33 | A |
| 1505 | CA | ASP | A | 1199 | −30.504 | 18.786 | 13.300 | 1.00 | 56.82 | A |
| 1506 | CB | ASP | A | 1199 | −30.241 | 19.132 | 11.795 | 1.00 | 57.28 | A |
| 1507 | CG | ASP | A | 1199 | −30.022 | 17.905 | 10.978 | 1.00 | 65.11 | A |
| 1508 | OD1 | ASP | A | 1199 | −31.067 | 17.408 | 10.423 | 1.00 | 73.36 | A |
| 1509 | OD2 | ASP | A | 1199 | −28.842 | 17.421 | 10.883 | 1.00 | 63.85 | A |
| 1510 | C | ASP | A | 1199 | −31.588 | 17.735 | 13.369 | 1.00 | 53.76 | A |
| 1511 | O | ASP | A | 1199 | −32.735 | 18.113 | 13.724 | 1.00 | 51.71 | A |
| 1512 | N | GLY | A | 1200 | −31.253 | 16.434 | 13.145 | 1.00 | 52.48 | A |
| 1513 | CA | GLY | A | 1200 | −32.309 | 15.383 | 13.271 | 1.00 | 53.35 | A |
| 1514 | C | GLY | A | 1200 | −31.821 | 14.263 | 12.443 | 1.00 | 57.34 | A |
| 1515 | O | GLY | A | 1200 | −32.587 | 13.247 | 12.142 | 1.00 | 58.17 | A |
| 1516 | N | ARG | A | 1201 | −30.526 | 14.424 | 12.070 | 1.00 | 58.58 | A |
| 1517 | CA | ARG | A | 1201 | −29.948 | 13.738 | 10.895 | 1.00 | 61.02 | A |
| 1518 | CB | ARG | A | 1201 | −29.876 | 14.699 | 9.716 | 1.00 | 58.55 | A |
| 1519 | CG | ARG | A | 1201 | −30.977 | 14.510 | 8.638 | 1.00 | 67.94 | A |
| 1520 | CD | ARG | A | 1201 | −30.594 | 15.246 | 7.136 | 1.00 | 68.54 | A |
| 1521 | NE | ARG | A | 1201 | −29.927 | 16.616 | 7.315 | 1.00 | 75.23 | A |
| 1522 | CZ | ARG | A | 1201 | −28.594 | 16.879 | 7.413 | 1.00 | 64.57 | A |
| 1523 | NH1 | ARG | A | 1201 | −28.148 | 18.121 | 7.508 | 1.00 | 57.11 | A |
| 1524 | NH2 | ARG | A | 1201 | −27.714 | 15.887 | 7.380 | 1.00 | 67.34 | A |
| 1525 | C | ARG | A | 1201 | −28.549 | 13.204 | 11.142 | 1.00 | 60.11 | A |
| 1526 | O | ARG | A | 1201 | −27.547 | 13.974 | 11.322 | 1.00 | 57.48 | A |
| 1527 | N | LEU | A | 1202 | −28.482 | 11.864 | 11.138 | 1.00 | 59.21 | A |
| 1528 | CA | LEU | A | 1202 | −27.194 | 11.167 | 11.058 | 1.00 | 54.32 | A |
| 1529 | CB | LEU | A | 1202 | −27.479 | 9.667 | 11.240 | 1.00 | 53.92 | A |
| 1530 | CG | LEU | A | 1202 | −28.179 | 8.895 | 12.287 | 1.00 | 51.46 | A |
| 1531 | CD1 | LEU | A | 1202 | −29.495 | 9.423 | 12.228 | 1.00 | 56.71 | A |
| 1532 | CD2 | LEU | A | 1202 | −28.310 | 7.474 | 11.874 | 1.00 | 53.16 | A |
| 1533 | C | LEU | A | 1202 | −26.380 | 11.277 | 9.687 | 1.00 | 51.88 | A |
| 1534 | O | LEU | A | 1202 | −26.938 | 11.070 | 8.649 | 1.00 | 47.85 | A |
| 1535 | N | VAL | A | 1203 | −25.047 | 11.193 | 9.767 | 1.00 | 51.85 | A |
| 1536 | CA | VAL | A | 1203 | −24.118 | 11.821 | 8.851 | 1.00 | 51.80 | A |
| 1537 | CB | VAL | A | 1203 | −24.084 | 13.332 | 9.156 | 1.00 | 52.63 | A |
| 1538 | CG1 | VAL | A | 1203 | −25.488 | 13.958 | 8.937 | 1.00 | 52.65 | A |
| 1539 | CG2 | VAL | A | 1203 | −23.515 | 13.612 | 10.681 | 1.00 | 52.59 | A |
| 1540 | C | VAL | A | 1203 | −22.732 | 11.374 | 9.186 | 1.00 | 51.71 | A |
| 1541 | O | VAL | A | 1203 | −22.408 | 11.269 | 10.363 | 1.00 | 50.79 | A |
| 1542 | N | ALA | A | 1204 | −21.901 | 11.197 | 8.144 | 1.00 | 52.43 | A |
| 1543 | CA | ALA | A | 1204 | −20.469 | 10.833 | 8.255 | 1.00 | 50.94 | A |
| 1544 | CB | ALA | A | 1204 | −19.645 | 11.141 | 6.963 | 1.00 | 51.03 | A |
| 1545 | C | ALA | A | 1204 | −19.971 | 11.634 | 9.395 | 1.00 | 51.08 | A |
| 1546 | O | ALA | A | 1204 | −20.803 | 12.117 | 10.196 | 1.00 | 49.64 | A |
| 1547 | N | ARG | A | 1205 | −18.648 | 11.618 | 9.560 | 1.00 | 51.32 | A |
| 1548 | CA | ARG | A | 1205 | −17.866 | 12.600 | 10.340 | 1.00 | 52.19 | A |
| 1549 | CB | ARG | A | 1205 | −16.604 | 12.890 | 9.460 | 1.00 | 52.76 | A |
| 1550 | CG | ARG | A | 1205 | −16.517 | 14.297 | 9.120 | 1.00 | 55.54 | A |
| 1551 | CD | ARG | A | 1205 | −17.431 | 14.648 | 7.859 | 1.00 | 57.71 | A |
| 1552 | NE | ARG | A | 1205 | −18.819 | 14.224 | 7.980 | 1.00 | 49.34 | A |
| 1553 | CZ | ARG | A | 1205 | −19.852 | 15.066 | 7.968 | 1.00 | 53.83 | A |
| 1554 | NH1 | ARG | A | 1205 | −19.701 | 16.437 | 7.859 | 1.00 | 47.61 | A |
| 1555 | NH2 | ARG | A | 1205 | −21.072 | 14.537 | 8.092 | 1.00 | 55.52 | A |
| 1556 | C | ARG | A | 1205 | −18.481 | 13.882 | 11.138 | 1.00 | 52.50 | A |
| 1557 | O | ARG | A | 1205 | −19.630 | 14.391 | 10.882 | 1.00 | 51.49 | A |
| 1558 | N | PRO | A | 1206 | −17.773 | 14.347 | 12.215 | 1.00 | 52.91 | A |
| 1559 | CD | PRO | A | 1206 | −16.572 | 13.895 | 12.947 | 1.00 | 55.09 | A |
| 1560 | CA | PRO | A | 1206 | −18.437 | 15.307 | 13.030 | 1.00 | 52.48 | A |
| 1561 | CB | PRO | A | 1206 | −18.163 | 14.787 | 14.470 | 1.00 | 50.04 | A |
| 1562 | CG | PRO | A | 1206 | −17.082 | 13.835 | 14.372 | 1.00 | 52.32 | A |
| 1563 | C | PRO | A | 1206 | −18.037 | 16.711 | 12.784 | 1.00 | 53.73 | A |
| 1564 | O | PRO | A | 1206 | −17.111 | 16.929 | 12.102 | 1.00 | 53.86 | A |
| 1565 | N | GLU | A | 1207 | −18.749 | 17.669 | 13.404 | 1.00 | 56.67 | A |
| 1566 | CA | GLU | A | 1207 | −18.880 | 19.077 | 12.955 | 1.00 | 57.30 | A |
| 1567 | CB | GLU | A | 1207 | −19.822 | 19.157 | 11.731 | 1.00 | 58.19 | A |
| 1568 | CG | GLU | A | 1207 | −21.327 | 18.841 | 12.005 | 1.00 | 56.23 | A |
| 1569 | CD | GLU | A | 1207 | −22.326 | 19.766 | 11.111 | 1.00 | 61.67 | A |
| 1570 | OE1 | GLU | A | 1207 | −23.538 | 19.360 | 10.991 | 1.00 | 61.69 | A |
| 1571 | OE2 | GLU | A | 1207 | −21.947 | 20.883 | 10.583 | 1.00 | 59.85 | A |
| 1572 | C | GLU | A | 1207 | −19.552 | 19.812 | 14.093 | 1.00 | 57.31 | A |
| 1573 | O | GLU | A | 1207 | −19.663 | 19.322 | 15.252 | 1.00 | 57.20 | A |
| 1574 | N | PRO | A | 1208 | −20.096 | 20.977 | 13.796 | 1.00 | 57.36 | A |
| 1575 | CD | PRO | A | 1208 | −20.662 | 21.741 | 12.645 | 1.00 | 57.99 | A |
| 1576 | CA | PRO | A | 1208 | −20.270 | 21.719 | 15.028 | 1.00 | 56.95 | A |
| 1577 | CB | PRO | A | 1208 | −20.819 | 23.079 | 14.493 | 1.00 | 57.92 | A |
| 1578 | CG | PRO | A | 1208 | −21.733 | 22.659 | 13.310 | 1.00 | 53.46 | A |
| 1579 | C | PRO | A | 1208 | −21.493 | 21.212 | 15.659 | 1.00 | 56.31 | A |
| 1580 | O | PRO | A | 1208 | −22.054 | 21.935 | 16.497 | 1.00 | 56.89 | A |
| 1581 | N | ALA | A | 1209 | −22.060 | 20.157 | 15.103 | 1.00 | 54.11 | A |
| 1582 | CA | ALA | A | 1209 | −23.453 | 20.008 | 15.369 | 1.00 | 53.49 | A |
| 1583 | CB | ALA | A | 1209 | −24.357 | 20.731 | 14.283 | 1.00 | 49.29 | A |
| 1584 | C | ALA | A | 1209 | −23.779 | 18.573 | 15.760 | 1.00 | 51.75 | A |
| 1585 | O | ALA | A | 1209 | −24.911 | 18.204 | 16.118 | 1.00 | 53.10 | A |
| 1586 | N | THR | A | 1210 | −22.739 | 17.813 | 15.858 | 1.00 | 49.97 | A |
| 1587 | CA | THR | A | 1210 | −22.914 | 16.486 | 16.357 | 1.00 | 52.14 | A |
| 1588 | CB | THR | A | 1210 | −22.094 | 15.481 | 15.503 | 1.00 | 50.07 | A |
| 1589 | OG1 | THR | A | 1210 | −20.835 | 16.066 | 15.224 | 1.00 | 56.16 | A |
| 1590 | CG2 | THR | A | 1210 | −22.618 | 15.278 | 14.201 | 1.00 | 42.68 | A |
| 1591 | C | THR | A | 1210 | −22.566 | 16.566 | 17.912 | 1.00 | 53.85 | A |
| 1592 | O | THR | A | 1210 | −23.134 | 15.860 | 18.707 | 1.00 | 55.38 | A |
| 1593 | N | GLY | A | 1211 | −21.681 | 17.459 | 18.354 | 1.00 | 54.89 | A |
| 1594 | CA | GLY | A | 1211 | −21.404 | 17.683 | 19.804 | 1.00 | 54.81 | A |
| 1595 | C | GLY | A | 1211 | −22.452 | 18.110 | 20.851 | 1.00 | 55.21 | A |
| 1596 | O | GLY | A | 1211 | −22.690 | 19.222 | 20.947 | 1.00 | 59.65 | A |
| 1597 | N | TYR | A | 1212 | −23.056 | 17.225 | 21.627 | 1.00 | 54.74 | A |
| 1598 | CA | TYR | A | 1212 | −24.015 | 17.410 | 22.764 | 1.00 | 52.12 | A |
| 1599 | CB | TYR | A | 1212 | −24.901 | 16.152 | 22.852 | 1.00 | 50.84 | A |
| 1600 | CG | TYR | A | 1212 | −25.707 | 15.987 | 21.534 | 1.00 | 47.51 | A |
| 1601 | CD1 | TYR | A | 1212 | −27.035 | 16.325 | 21.468 | 1.00 | 45.57 | A |
| 1602 | CE1 | TYR | A | 1212 | −27.764 | 16.298 | 20.280 | 1.00 | 48.73 | A |
| 1603 | CD2 | TYR | A | 1212 | −25.157 | 15.454 | 20.416 | 1.00 | 48.94 | A |
| 1604 | CE2 | TYR | A | 1212 | −25.919 | 15.319 | 19.188 | 1.00 | 46.71 | A |
| 1605 | CZ | TYR | A | 1212 | −27.166 | 15.854 | 19.138 | 1.00 | 45.21 | A |
| 1606 | OH | TYR | A | 1212 | −27.915 | 15.710 | 18.082 | 1.00 | 44.50 | A |
| 1607 | C | TYR | A | 1212 | −23.126 | 17.408 | 23.971 | 1.00 | 50.81 | A |
| 1608 | O | TYR | A | 1212 | −21.983 | 16.861 | 23.870 | 1.00 | 51.41 | A |
| 1609 | N | THR | A | 1213 | −23.487 | 18.137 | 25.022 | 1.00 | 48.87 | A |
| 1610 | CA | THR | A | 1213 | −22.679 | 18.065 | 26.310 | 1.00 | 47.53 | A |
| 1611 | CB | THR | A | 1213 | −22.625 | 19.369 | 26.957 | 1.00 | 46.92 | A |
| 1612 | OG1 | THR | A | 1213 | −23.964 | 19.815 | 26.910 | 1.00 | 45.96 | A |
| 1613 | CG2 | THR | A | 1213 | −21.770 | 20.460 | 26.116 | 1.00 | 49.78 | A |
| 1614 | C | THR | A | 1213 | −23.682 | 17.283 | 27.184 | 1.00 | 46.37 | A |
| 1615 | O | THR | A | 1213 | −24.877 | 17.668 | 27.093 | 1.00 | 43.28 | A |
| 1616 | N | LEU | A | 1214 | −23.323 | 16.173 | 27.893 | 1.00 | 45.11 | A |
| 1617 | CA | LEU | A | 1214 | −24.422 | 15.410 | 28.472 | 1.00 | 47.58 | A |
| 1618 | CB | LEU | A | 1214 | −24.060 | 13.972 | 28.980 | 1.00 | 47.46 | A |
| 1619 | CG | LEU | A | 1214 | −23.257 | 13.367 | 27.837 | 1.00 | 47.30 | A |
| 1620 | CD1 | LEU | A | 1214 | −22.625 | 11.998 | 28.171 | 1.00 | 46.55 | A |
| 1621 | CD2 | LEU | A | 1214 | −24.073 | 13.366 | 26.521 | 1.00 | 36.33 | A |
| 1622 | C | LEU | A | 1214 | −24.853 | 16.249 | 29.581 | 1.00 | 48.16 | A |
| 1623 | O | LEU | A | 1214 | −24.097 | 16.935 | 30.160 | 1.00 | 49.60 | A |
| 1624 | N | GLU | A | 1215 | −26.078 | 16.198 | 29.941 | 1.00 | 50.93 | A |
| 1625 | CA | GLU | A | 1215 | −26.386 | 16.767 | 31.236 | 1.00 | 53.09 | A |
| 1626 | CB | GLU | A | 1215 | −27.185 | 18.050 | 31.056 | 1.00 | 53.78 | A |
| 1627 | CG | GLU | A | 1215 | −27.118 | 19.040 | 32.391 | 1.00 | 55.21 | A |
| 1628 | CD | GLU | A | 1215 | −27.717 | 20.421 | 32.100 | 1.00 | 54.06 | A |
| 1629 | OE1 | GLU | A | 1215 | −26.962 | 21.324 | 31.670 | 1.00 | 48.79 | A |
| 1630 | OE2 | GLU | A | 1215 | −28.989 | 20.505 | 32.131 | 1.00 | 54.62 | A |
| 1631 | C | GLU | A | 1215 | −27.069 | 15.835 | 32.274 | 1.00 | 51.25 | A |
| 1632 | O | GLU | A | 1215 | −28.236 | 15.628 | 32.215 | 1.00 | 53.62 | A |
| 1633 | N | PHE | A | 1216 | −26.345 | 15.299 | 33.222 | 1.00 | 49.48 | A |
| 1634 | CA | PHE | A | 1216 | −26.945 | 14.350 | 34.164 | 1.00 | 50.28 | A |
| 1635 | CB | PHE | A | 1216 | −25.810 | 13.593 | 34.828 | 1.00 | 51.32 | A |
| 1636 | CG | PHE | A | 1216 | −24.879 | 12.932 | 33.818 | 1.00 | 44.77 | A |
| 1637 | CD1 | PHE | A | 1216 | −25.304 | 11.925 | 33.080 | 1.00 | 39.43 | A |
| 1638 | CD2 | PHE | A | 1216 | −23.717 | 13.434 | 33.552 | 1.00 | 38.47 | A |
| 1639 | CE1 | PHE | A | 1216 | −24.535 | 11.270 | 32.226 | 1.00 | 40.89 | A |
| 1640 | CE2 | PHE | A | 1216 | −22.973 | 12.829 | 32.595 | 1.00 | 47.64 | A |
| 1641 | CZ | PHE | A | 1216 | −23.417 | 11.677 | 31.937 | 1.00 | 44.22 | A |
| 1642 | C | PHE | A | 1216 | −27.925 | 14.915 | 35.208 | 1.00 | 51.91 | A |
| 1643 | O | PHE | A | 1216 | −27.839 | 16.106 | 35.541 | 1.00 | 51.72 | A |
| 1644 | N | ARG | A | 1217 | −28.937 | 14.092 | 35.557 | 1.00 | 51.98 | A |
| 1645 | CA | ARG | A | 1217 | −29.971 | 14.333 | 36.585 | 1.00 | 53.35 | A |
| 1646 | CB | ARG | A | 1217 | −31.237 | 14.945 | 36.034 | 1.00 | 51.43 | A |
| 1647 | CG | ARG | A | 1217 | −30.889 | 16.150 | 35.444 | 1.00 | 54.21 | A |
| 1648 | CD | ARG | A | 1217 | −31.960 | 17.081 | 35.339 | 1.00 | 60.03 | A |
| 1649 | NE | ARG | A | 1217 | −31.302 | 18.289 | 34.866 | 1.00 | 61.93 | A |
| 1650 | CZ | ARG | A | 1217 | −31.645 | 19.511 | 35.201 | 1.00 | 61.49 | A |
| 1651 | NH1 | ARG | A | 1217 | −32.685 | 19.696 | 36.019 | 1.00 | 58.97 | A |
| 1652 | NH2 | ARG | A | 1217 | −30.956 | 20.538 | 34.687 | 1.00 | 62.07 | A |
| 1653 | C | ARG | A | 1217 | −30.383 | 13.014 | 37.154 | 1.00 | 56.64 | A |
| 1654 | O | ARG | A | 1217 | −31.225 | 12.298 | 36.548 | 1.00 | 57.70 | A |
| 1655 | N | SER | A | 1218 | −29.773 | 12.681 | 38.304 | 1.00 | 58.24 | A |
| 1656 | CA | SER | A | 1218 | −29.926 | 11.348 | 39.039 | 1.00 | 56.57 | A |
| 1657 | CB | SER | A | 1218 | −30.752 | 11.666 | 40.306 | 1.00 | 55.69 | A |
| 1658 | OG | SER | A | 1218 | −31.816 | 12.506 | 39.736 | 1.00 | 51.77 | A |
| 1659 | C | SER | A | 1218 | −30.724 | 10.360 | 38.149 | 1.00 | 55.12 | A |
| 1660 | O | SER | A | 1218 | −31.878 | 10.612 | 37.940 | 1.00 | 56.40 | A |
| 1661 | N | GLY | A | 1219 | −30.121 | 9.296 | 37.624 | 1.00 | 53.88 | A |
| 1662 | CA | GLY | A | 1219 | −30.776 | 8.210 | 36.790 | 1.00 | 52.47 | A |
| 1663 | C | GLY | A | 1219 | −30.622 | 8.628 | 35.374 | 1.00 | 52.43 | A |
| 1664 | O | GLY | A | 1219 | −30.164 | 7.889 | 34.514 | 1.00 | 51.68 | A |
| 1665 | N | LYS | A | 1220 | −30.886 | 9.904 | 35.162 | 1.00 | 52.46 | A |
| 1666 | CA | LYS | A | 1220 | −31.091 | 10.375 | 33.914 | 1.00 | 52.88 | A |
| 1667 | CB | LYS | A | 1220 | −32.363 | 11.036 | 33.961 | 1.00 | 53.57 | A |
| 1668 | CG | LYS | A | 1220 | −33.269 | 10.254 | 34.912 | 1.00 | 47.05 | A |
| 1669 | CD | LYS | A | 1220 | −34.404 | 9.638 | 34.171 | 1.00 | 55.99 | A |
| 1670 | CE | LYS | A | 1220 | −34.097 | 8.097 | 34.168 | 1.00 | 56.35 | A |
| 1671 | NZ | LYS | A | 1220 | −34.452 | 7.630 | 35.532 | 1.00 | 44.34 | A |
| 1672 | C | LYS | A | 1220 | −30.055 | 11.302 | 33.422 | 1.00 | 56.04 | A |
| 1673 | O | LYS | A | 1220 | −29.263 | 11.903 | 34.295 | 1.00 | 57.59 | A |
| 1674 | N | VAL | A | 1221 | −30.049 | 11.412 | 32.048 | 1.00 | 54.93 | A |
| 1675 | CA | VAL | A | 1221 | −29.161 | 12.348 | 31.315 | 1.00 | 54.81 | A |
| 1676 | CB | VAL | A | 1221 | −27.977 | 11.581 | 30.743 | 1.00 | 53.65 | A |
| 1677 | CG1 | VAL | A | 1221 | −28.445 | 10.685 | 29.696 | 1.00 | 55.71 | A |
| 1678 | CG2 | VAL | A | 1221 | −26.945 | 12.424 | 30.174 | 1.00 | 53.99 | A |
| 1679 | C | VAL | A | 1221 | −30.000 | 13.019 | 30.237 | 1.00 | 54.84 | A |
| 1680 | O | VAL | A | 1221 | −31.044 | 12.458 | 29.739 | 1.00 | 55.57 | A |
| 1681 | N | ALA | A | 1222 | −29.535 | 14.188 | 29.857 | 1.00 | 53.40 | A |
| 1682 | CA | ALA | A | 1222 | −30.201 | 14.972 | 28.807 | 1.00 | 53.03 | A |
| 1683 | CB | ALA | A | 1222 | −31.095 | 15.978 | 29.411 | 1.00 | 52.24 | A |
| 1684 | C | ALA | A | 1222 | −29.128 | 15.653 | 27.874 | 1.00 | 53.61 | A |
| 1685 | O | ALA | A | 1222 | −27.847 | 15.720 | 28.166 | 1.00 | 51.21 | A |
| 1686 | N | PHE | A | 1223 | −29.572 | 16.126 | 26.699 | 1.00 | 52.35 | A |
| 1687 | CA | PHE | A | 1223 | −28.453 | 16.475 | 25.801 | 1.00 | 52.12 | A |
| 1688 | CB | PHE | A | 1223 | −28.369 | 15.436 | 24.698 | 1.00 | 48.73 | A |
| 1689 | CG | PHE | A | 1223 | −28.331 | 14.009 | 25.158 | 1.00 | 49.43 | A |
| 1690 | CD1 | PHE | A | 1223 | −27.119 | 13.413 | 25.578 | 1.00 | 49.37 | A |
| 1691 | CD2 | PHE | A | 1223 | −29.436 | 13.171 | 24.885 | 1.00 | 51.43 | A |
| 1692 | CE1 | PHE | A | 1223 | −26.963 | 12.053 | 25.739 | 1.00 | 42.48 | A |
| 1693 | CE2 | PHE | A | 1223 | −29.331 | 11.813 | 25.010 | 1.00 | 49.72 | A |
| 1694 | CZ | PHE | A | 1223 | −28.017 | 11.244 | 25.477 | 1.00 | 52.67 | A |
| 1695 | C | PHE | A | 1223 | −28.414 | 17.823 | 25.132 | 1.00 | 51.66 | A |
| 1696 | O | PHE | A | 1223 | −29.377 | 18.148 | 24.380 | 1.00 | 49.85 | A |
| 1697 | N | ARG | A | 1224 | −27.278 | 18.543 | 25.239 | 1.00 | 51.48 | A |
| 1698 | CA | ARG | A | 1224 | −27.383 | 19.908 | 24.713 | 1.00 | 52.69 | A |
| 1699 | CB | ARG | A | 1224 | −27.162 | 21.059 | 25.678 | 1.00 | 50.65 | A |
| 1700 | CG | ARG | A | 1224 | −25.876 | 21.627 | 25.478 | 1.00 | 48.65 | A |
| 1701 | CD | ARG | A | 1224 | −25.529 | 23.029 | 26.240 | 1.00 | 53.91 | A |
| 1702 | NE | ARG | A | 1224 | −26.479 | 23.203 | 27.337 | 1.00 | 60.13 | A |
| 1703 | CZ | ARG | A | 1224 | −26.928 | 24.344 | 27.845 | 1.00 | 53.57 | A |
| 1704 | NH1 | ARG | A | 1224 | −26.517 | 25.547 | 27.335 | 1.00 | 51.86 | A |
| 1705 | NH2 | ARG | A | 1224 | −27.827 | 24.208 | 28.833 | 1.00 | 45.78 | A |
| 1706 | C | ARG | A | 1224 | −26.449 | 20.125 | 23.730 | 1.00 | 56.06 | A |
| 1707 | O | ARG | A | 1224 | −25.191 | 20.166 | 24.025 | 1.00 | 57.79 | A |
| 1708 | N | ASP | A | 1225 | −27.066 | 20.351 | 22.552 | 1.00 | 59.47 | A |
| 1709 | CA | ASP | A | 1225 | −26.386 | 20.698 | 21.334 | 1.00 | 61.55 | A |
| 1710 | CB | ASP | A | 1225 | −27.380 | 20.797 | 20.238 | 1.00 | 60.84 | A |
| 1711 | CG | ASP | A | 1225 | −27.774 | 22.252 | 19.950 | 1.00 | 61.50 | A |
| 1712 | OD1 | ASP | A | 1225 | −26.910 | 23.176 | 19.861 | 1.00 | 49.06 | A |
| 1713 | OD2 | ASP | A | 1225 | −28.995 | 22.465 | 19.746 | 1.00 | 65.92 | A |
| 1714 | C | ASP | A | 1225 | −25.707 | 22.091 | 21.525 | 1.00 | 64.13 | A |
| 1715 | O | ASP | A | 1225 | −26.062 | 22.810 | 22.510 | 1.00 | 66.64 | A |
| 1716 | N | CYS | A | 1226 | −24.784 | 22.453 | 20.577 | 1.00 | 63.90 | A |
| 1717 | CA | CYS | A | 1226 | −23.990 | 23.690 | 20.475 | 1.00 | 62.63 | A |
| 1718 | CB | CYS | A | 1226 | −23.120 | 23.600 | 19.202 | 1.00 | 63.70 | A |
| 1719 | SG | CYS | A | 1226 | −24.122 | 23.317 | 17.556 | 1.00 | 57.17 | A |
| 1720 | C | CYS | A | 1226 | −24.792 | 25.019 | 20.380 | 1.00 | 64.24 | A |
| 1721 | O | CYS | A | 1226 | −24.242 | 26.093 | 20.008 | 1.00 | 66.01 | A |
| 1722 | N | GLU | A | 1227 | −26.087 | 24.954 | 20.583 | 1.00 | 62.70 | A |
| 1723 | CA | GLU | A | 1227 | −26.879 | 26.150 | 20.470 | 1.00 | 63.51 | A |
| 1724 | CB | GLU | A | 1227 | −27.978 | 26.040 | 19.316 | 1.00 | 63.57 | A |
| 1725 | CG | GLU | A | 1227 | −27.673 | 26.251 | 17.740 | 1.00 | 59.82 | A |
| 1726 | CD | GLU | A | 1227 | −26.399 | 27.039 | 17.195 | 1.00 | 63.84 | A |
| 1727 | OE1 | GLU | A | 1227 | −26.159 | 28.300 | 17.415 | 1.00 | 66.16 | A |
| 1728 | OE2 | GLU | A | 1227 | −25.682 | 26.390 | 16.377 | 1.00 | 56.40 | A |
| 1729 | C | GLU | A | 1227 | −27.511 | 26.442 | 21.914 | 1.00 | 64.20 | A |
| 1730 | O | GLU | A | 1227 | −27.341 | 27.561 | 22.455 | 1.00 | 65.41 | A |
| 1731 | N | GLY | A | 1228 | −28.217 | 25.437 | 22.479 | 1.00 | 61.97 | A |
| 1732 | CA | GLY | A | 1228 | −28.691 | 25.361 | 23.838 | 1.00 | 59.70 | A |
| 1733 | C | GLY | A | 1228 | −30.035 | 24.672 | 23.702 | 1.00 | 57.81 | A |
| 1734 | O | GLY | A | 1228 | −31.014 | 25.110 | 24.183 | 1.00 | 58.22 | A |
| 1735 | N | ARG | A | 1229 | −30.128 | 23.535 | 23.093 | 1.00 | 57.52 | A |
| 1736 | CA | ARG | A | 1229 | −31.410 | 23.198 | 22.611 | 1.00 | 58.85 | A |
| 1737 | CB | ARG | A | 1229 | −31.511 | 23.511 | 21.087 | 1.00 | 58.72 | A |
| 1738 | CG | ARG | A | 1229 | −30.657 | 24.657 | 20.382 | 1.00 | 57.38 | A |
| 1739 | CD | ARG | A | 1229 | −31.085 | 24.645 | 18.817 | 1.00 | 62.21 | A |
| 1740 | NE | ARG | A | 1229 | −30.826 | 23.370 | 18.065 | 1.00 | 77.52 | A |
| 1741 | CZ | ARG | A | 1229 | −31.666 | 22.731 | 17.183 | 1.00 | 79.23 | A |
| 1742 | NH1 | ARG | A | 1229 | −32.919 | 23.205 | 16.915 | 1.00 | 80.63 | A |
| 1743 | NH2 | ARG | A | 1229 | −31.267 | 21.593 | 16.560 | 1.00 | 71.78 | A |
| 1744 | C | ARG | A | 1229 | −31.585 | 21.731 | 22.761 | 1.00 | 60.80 | A |
| 1745 | O | ARG | A | 1229 | −31.846 | 21.012 | 21.724 | 1.00 | 58.38 | A |
| 1746 | N | TYR | A | 1230 | −31.352 | 21.278 | 24.016 | 1.00 | 62.14 | A |
| 1747 | CA | TYR | A | 1230 | −31.724 | 19.926 | 24.498 | 1.00 | 63.40 | A |
| 1748 | CB | TYR | A | 1230 | −32.505 | 19.984 | 25.791 | 1.00 | 64.04 | A |
| 1749 | CG | TYR | A | 1230 | −31.560 | 20.508 | 26.886 | 1.00 | 68.93 | A |
| 1750 | CD1 | TYR | A | 1230 | −31.649 | 21.849 | 27.351 | 1.00 | 60.29 | A |
| 1751 | CE1 | TYR | A | 1230 | −30.706 | 22.319 | 28.340 | 1.00 | 70.46 | A |
| 1752 | CD2 | TYR | A | 1230 | −30.486 | 19.643 | 27.426 | 1.00 | 71.05 | A |
| 1753 | CE2 | TYR | A | 1230 | −29.515 | 20.110 | 28.438 | 1.00 | 67.36 | A |
| 1754 | CZ | TYR | A | 1230 | −29.657 | 21.448 | 28.963 | 1.00 | 70.91 | A |
| 1755 | OH | TYR | A | 1230 | −28.779 | 21.980 | 30.010 | 1.00 | 64.46 | A |
| 1756 | C | TYR | A | 1230 | −32.314 | 18.885 | 23.556 | 1.00 | 62.43 | A |
| 1757 | O | TYR | A | 1230 | −33.344 | 19.085 | 23.002 | 1.00 | 60.96 | A |
| 1758 | N | LEU | A | 1231 | −31.604 | 17.765 | 23.393 | 1.00 | 62.11 | A |
| 1759 | CA | LEU | A | 1231 | −32.144 | 16.680 | 22.597 | 1.00 | 62.94 | A |
| 1760 | CB | LEU | A | 1231 | −31.138 | 15.474 | 22.496 | 1.00 | 62.84 | A |
| 1761 | CG | LEU | A | 1231 | −30.967 | 14.839 | 21.011 | 1.00 | 68.34 | A |
| 1762 | CD1 | LEU | A | 1231 | −31.049 | 15.722 | 19.668 | 1.00 | 49.27 | A |
| 1763 | CD2 | LEU | A | 1231 | −29.621 | 14.058 | 20.866 | 1.00 | 64.93 | A |
| 1764 | C | LEU | A | 1231 | −33.637 | 16.268 | 22.952 | 1.00 | 60.44 | A |
| 1765 | O | LEU | A | 1231 | −34.103 | 16.445 | 24.110 | 1.00 | 60.78 | A |
| 1766 | N | ALA | A | 1232 | −34.376 | 15.787 | 21.965 | 1.00 | 56.47 | A |
| 1767 | CA | ALA | A | 1232 | −35.549 | 15.075 | 22.299 | 1.00 | 56.66 | A |
| 1768 | CB | ALA | A | 1232 | −36.396 | 15.853 | 23.326 | 1.00 | 58.67 | A |
| 1769 | C | ALA | A | 1232 | −36.392 | 14.584 | 21.150 | 1.00 | 56.19 | A |
| 1770 | O | ALA | A | 1232 | −36.322 | 15.235 | 20.104 | 1.00 | 54.58 | A |
| 1771 | N | PRO | A | 1233 | −37.202 | 13.469 | 21.368 | 1.00 | 55.23 | A |
| 1772 | CD | PRO | A | 1233 | −37.341 | 12.737 | 22.616 | 1.00 | 53.53 | A |
| 1773 | CA | PRO | A | 1233 | −38.094 | 12.859 | 20.383 | 1.00 | 58.42 | A |
| 1774 | CB | PRO | A | 1233 | −38.918 | 11.848 | 21.228 | 1.00 | 56.20 | A |
| 1775 | CG | PRO | A | 1233 | −38.033 | 11.492 | 22.230 | 1.00 | 52.89 | A |
| 1776 | C | PRO | A | 1233 | −38.966 | 13.875 | 19.482 | 1.00 | 59.90 | A |
| 1777 | O | PRO | A | 1233 | −38.665 | 15.075 | 19.450 | 1.00 | 59.94 | A |
| 1778 | N | SER | A | 1234 | −39.973 | 13.399 | 18.726 | 1.00 | 61.92 | A |
| 1779 | CA | SER | A | 1234 | −40.745 | 14.261 | 17.764 | 1.00 | 62.11 | A |
| 1780 | CB | SER | A | 1234 | −39.910 | 14.680 | 16.570 | 1.00 | 62.78 | A |
| 1781 | OG | SER | A | 1234 | −38.591 | 15.052 | 17.020 | 1.00 | 59.06 | A |
| 1782 | C | SER | A | 1234 | −42.019 | 13.671 | 17.312 | 1.00 | 62.46 | A |
| 1783 | O | SER | A | 1234 | −43.014 | 13.918 | 17.899 | 1.00 | 65.41 | A |
| 1784 | N | GLY | A | 1235 | −42.044 | 12.882 | 16.286 | 1.00 | 63.33 | A |
| 1785 | CA | GLY | A | 1235 | −43.364 | 12.283 | 15.966 | 1.00 | 64.32 | A |
| 1786 | C | GLY | A | 1235 | −43.694 | 11.018 | 16.759 | 1.00 | 63.69 | A |
| 1787 | O | GLY | A | 1235 | −43.210 | 10.817 | 17.916 | 1.00 | 63.28 | A |
| 1788 | N | PRO | A | 1236 | −44.470 | 10.151 | 16.121 | 1.00 | 64.01 | A |
| 1789 | CD | PRO | A | 1236 | −45.135 | 10.673 | 14.917 | 1.00 | 65.58 | A |
| 1790 | CA | PRO | A | 1236 | −44.842 | 8.736 | 16.373 | 1.00 | 65.05 | A |
| 1791 | CB | PRO | A | 1236 | −45.902 | 8.402 | 15.264 | 1.00 | 64.27 | A |
| 1792 | CG | PRO | A | 1236 | −45.534 | 9.403 | 14.139 | 1.00 | 68.08 | A |
| 1793 | C | PRO | A | 1236 | −43.622 | 7.856 | 16.183 | 1.00 | 62.98 | A |
| 1794 | O | PRO | A | 1236 | −43.656 | 6.633 | 16.448 | 1.00 | 63.68 | A |
| 1795 | N | SER | A | 1237 | −42.578 | 8.505 | 15.744 | 1.00 | 61.00 | A |
| 1796 | CA | SER | A | 1237 | −41.290 | 7.869 | 15.651 | 1.00 | 62.60 | A |
| 1797 | CB | SER | A | 1237 | −40.895 | 7.865 | 14.163 | 1.00 | 64.27 | A |
| 1798 | OG | SER | A | 1237 | −40.943 | 9.237 | 13.597 | 1.00 | 64.10 | A |
| 1799 | C | SER | A | 1237 | −40.237 | 8.694 | 16.508 | 1.00 | 61.96 | A |
| 1800 | O | SER | A | 1237 | −39.243 | 9.190 | 15.990 | 1.00 | 61.05 | A |
| 1801 | N | GLY | A | 1238 | −40.554 | 8.921 | 17.784 | 1.00 | 60.38 | A |
| 1802 | CA | GLY | A | 1238 | −39.671 | 9.513 | 18.720 | 1.00 | 57.37 | A |
| 1803 | C | GLY | A | 1238 | −38.510 | 10.149 | 17.985 | 1.00 | 56.67 | A |
| 1804 | O | GLY | A | 1238 | −37.451 | 10.299 | 18.619 | 1.00 | 54.97 | A |
| 1805 | N | THR | A | 1239 | −38.706 | 10.582 | 16.706 | 1.00 | 52.71 | A |
| 1806 | CA | THR | A | 1239 | −37.504 | 10.984 | 15.888 | 1.00 | 51.87 | A |
| 1807 | CB | THR | A | 1239 | −37.858 | 11.736 | 14.581 | 1.00 | 48.97 | A |
| 1808 | OG1 | THR | A | 1239 | −38.327 | 10.730 | 13.687 | 1.00 | 58.00 | A |
| 1809 | CG2 | THR | A | 1239 | −36.741 | 12.376 | 13.931 | 1.00 | 43.00 | A |
| 1810 | C | THR | A | 1239 | −36.611 | 11.690 | 16.786 | 1.00 | 52.07 | A |
| 1811 | O | THR | A | 1239 | −37.025 | 12.674 | 17.387 | 1.00 | 54.36 | A |
| 1812 | N | LEU | A | 1240 | −35.409 | 11.193 | 16.937 | 1.00 | 53.28 | A |
| 1813 | CA | LEU | A | 1240 | −34.529 | 11.753 | 17.950 | 1.00 | 55.51 | A |
| 1814 | CB | LEU | A | 1240 | −33.539 | 10.706 | 18.581 | 1.00 | 54.14 | A |
| 1815 | CG | LEU | A | 1240 | −33.452 | 10.719 | 20.169 | 1.00 | 56.36 | A |
| 1816 | CD1 | LEU | A | 1240 | −32.245 | 10.007 | 20.773 | 1.00 | 47.97 | A |
| 1817 | CD2 | LEU | A | 1240 | −33.507 | 12.159 | 20.957 | 1.00 | 49.51 | A |
| 1818 | C | LEU | A | 1240 | −33.852 | 13.029 | 17.453 | 1.00 | 56.46 | A |
| 1819 | O | LEU | A | 1240 | −32.945 | 12.943 | 16.765 | 1.00 | 59.82 | A |
| 1820 | N | LYS | A | 1241 | −34.324 | 14.220 | 17.790 | 1.00 | 57.68 | A |
| 1821 | CA | LYS | A | 1241 | −33.699 | 15.432 | 17.313 | 1.00 | 58.06 | A |
| 1822 | CB | LYS | A | 1241 | −34.521 | 15.829 | 16.124 | 1.00 | 59.73 | A |
| 1823 | CG | LYS | A | 1241 | −36.042 | 15.557 | 16.392 | 1.00 | 58.68 | A |
| 1824 | CD | LYS | A | 1241 | −36.744 | 15.539 | 15.078 | 1.00 | 57.25 | A |
| 1825 | CE | LYS | A | 1241 | −37.465 | 16.766 | 14.782 | 1.00 | 53.04 | A |
| 1826 | NZ | LYS | A | 1241 | −38.806 | 16.209 | 15.102 | 1.00 | 57.12 | A |
| 1827 | C | LYS | A | 1241 | −33.760 | 16.622 | 18.295 | 1.00 | 58.42 | A |
| 1828 | O | LYS | A | 1241 | −34.829 | 16.904 | 18.813 | 1.00 | 57.40 | A |
| 1829 | N | ALA | A | 1242 | −32.651 | 17.338 | 18.519 | 1.00 | 58.72 | A |
| 1830 | CA | ALA | A | 1242 | −32.700 | 18.611 | 19.274 | 1.00 | 58.71 | A |
| 1831 | CB | ALA | A | 1242 | −31.877 | 19.664 | 18.641 | 1.00 | 59.35 | A |
| 1832 | C | ALA | A | 1242 | −34.071 | 19.163 | 19.476 | 1.00 | 58.72 | A |
| 1833 | O | ALA | A | 1242 | −35.061 | 18.513 | 19.144 | 1.00 | 58.95 | A |
| 1834 | N | GLY | A | 1243 | −34.121 | 20.370 | 20.029 | 1.00 | 59.24 | A |
| 1835 | CA | GLY | A | 1243 | −35.390 | 20.940 | 20.550 | 1.00 | 62.05 | A |
| 1836 | C | GLY | A | 1243 | −35.230 | 22.391 | 20.988 | 1.00 | 62.71 | A |
| 1837 | O | GLY | A | 1243 | −34.161 | 22.914 | 21.028 | 1.00 | 61.95 | A |
| 1838 | N | LYS | A | 1244 | −36.310 | 23.031 | 21.362 | 1.00 | 64.53 | A |
| 1839 | CA | LYS | A | 1244 | −36.258 | 24.499 | 21.557 | 1.00 | 64.63 | A |
| 1840 | CB | LYS | A | 1244 | −37.358 | 25.185 | 20.662 | 1.00 | 63.49 | A |
| 1841 | CG | LYS | A | 1244 | −38.689 | 24.577 | 20.647 | 1.00 | 53.76 | A |
| 1842 | CD | LYS | A | 1244 | −38.904 | 23.772 | 19.420 | 1.00 | 54.27 | A |
| 1843 | CE | LYS | A | 1244 | −38.949 | 24.585 | 18.162 | 1.00 | 53.37 | A |
| 1844 | NZ | LYS | A | 1244 | −37.676 | 25.383 | 17.661 | 1.00 | 58.03 | A |
| 1845 | C | LYS | A | 1244 | −36.517 | 24.872 | 23.040 | 1.00 | 65.49 | A |
| 1846 | O | LYS | A | 1244 | −36.531 | 26.035 | 23.357 | 1.00 | 65.75 | A |
| 1847 | N | ALA | A | 1245 | −36.782 | 23.870 | 23.897 | 1.00 | 65.90 | A |
| 1848 | CA | ALA | A | 1245 | −37.144 | 24.015 | 25.293 | 1.00 | 63.80 | A |
| 1849 | CB | ALA | A | 1245 | −37.487 | 22.643 | 25.817 | 1.00 | 64.64 | A |
| 1850 | C | ALA | A | 1245 | −35.977 | 24.605 | 26.034 | 1.00 | 63.72 | A |
| 1851 | O | ALA | A | 1245 | −34.834 | 24.131 | 25.856 | 1.00 | 59.78 | A |
| 1852 | N | THR | A | 1246 | −36.277 | 25.710 | 26.793 | 1.00 | 65.16 | A |
| 1853 | CA | THR | A | 1246 | −35.294 | 26.440 | 27.694 | 1.00 | 63.05 | A |
| 1854 | CB | THR | A | 1246 | −35.988 | 27.671 | 28.557 | 1.00 | 64.23 | A |
| 1855 | OG1 | THR | A | 1246 | −37.230 | 27.288 | 29.180 | 1.00 | 64.17 | A |
| 1856 | CG2 | THR | A | 1246 | −36.225 | 29.167 | 27.837 | 1.00 | 58.43 | A |
| 1857 | C | THR | A | 1246 | −34.447 | 25.370 | 28.600 | 1.00 | 63.72 | A |
| 1858 | O | THR | A | 1246 | −33.210 | 25.057 | 28.348 | 1.00 | 63.78 | A |
| 1859 | N | LYS | A | 1247 | −35.098 | 24.762 | 29.594 | 1.00 | 63.32 | A |
| 1860 | CA | LYS | A | 1247 | −34.387 | 23.892 | 30.597 | 1.00 | 63.10 | A |
| 1861 | CB | LYS | A | 1247 | −34.435 | 24.517 | 32.077 | 1.00 | 63.59 | A |
| 1862 | CG | LYS | A | 1247 | −34.078 | 26.011 | 32.364 | 1.00 | 62.45 | A |
| 1863 | CD | LYS | A | 1247 | −34.834 | 26.588 | 33.605 | 1.00 | 61.45 | A |
| 1864 | CE | LYS | A | 1247 | −34.143 | 27.926 | 34.330 | 1.00 | 65.20 | A |
| 1865 | NZ | LYS | A | 1247 | −34.593 | 28.690 | 35.811 | 1.00 | 58.00 | A |
| 1866 | C | LYS | A | 1247 | −35.194 | 22.545 | 30.668 | 1.00 | 62.81 | A |
| 1867 | O | LYS | A | 1247 | −36.434 | 22.602 | 30.831 | 1.00 | 63.10 | A |
| 1868 | N | VAL | A | 1248 | −34.493 | 21.408 | 30.725 | 1.00 | 61.20 | A |
| 1869 | CA | VAL | A | 1248 | −34.952 | 20.079 | 30.459 | 1.00 | 60.29 | A |
| 1870 | CB | VAL | A | 1248 | −33.747 | 19.025 | 30.724 | 1.00 | 61.88 | A |
| 1871 | CG1 | VAL | A | 1248 | −33.459 | 18.585 | 32.230 | 1.00 | 64.28 | A |
| 1872 | CG2 | VAL | A | 1248 | −33.720 | 17.707 | 29.743 | 1.00 | 65.29 | A |
| 1873 | C | VAL | A | 1248 | −36.255 | 19.862 | 31.165 | 1.00 | 60.53 | A |
| 1874 | O | VAL | A | 1248 | −36.726 | 20.796 | 31.900 | 1.00 | 61.32 | A |
| 1875 | N | GLY | A | 1249 | −36.895 | 18.691 | 30.957 | 1.00 | 58.00 | A |
| 1876 | CA | GLY | A | 1249 | −38.144 | 18.417 | 31.669 | 1.00 | 54.79 | A |
| 1877 | C | GLY | A | 1249 | −38.186 | 16.975 | 31.499 | 1.00 | 55.25 | A |
| 1878 | O | GLY | A | 1249 | −37.103 | 16.460 | 31.164 | 1.00 | 55.94 | A |
| 1879 | N | LYS | A | 1250 | −39.372 | 16.337 | 31.670 | 1.00 | 56.52 | A |
| 1880 | CA | LYS | A | 1250 | −39.627 | 14.857 | 31.451 | 1.00 | 58.35 | A |
| 1881 | CB | LYS | A | 1250 | −41.018 | 14.361 | 31.847 | 1.00 | 60.21 | A |
| 1882 | CG | LYS | A | 1250 | −42.211 | 14.946 | 31.038 | 1.00 | 57.62 | A |
| 1883 | CD | LYS | A | 1250 | −43.590 | 14.589 | 31.745 | 1.00 | 54.39 | A |
| 1884 | CE | LYS | A | 1250 | −43.973 | 15.513 | 32.923 | 1.00 | 45.38 | A |
| 1885 | NZ | LYS | A | 1250 | −43.579 | 16.997 | 32.959 | 1.00 | 47.32 | A |
| 1886 | C | LYS | A | 1250 | −39.468 | 14.356 | 30.038 | 1.00 | 61.04 | A |
| 1887 | O | LYS | A | 1250 | −38.532 | 13.615 | 29.818 | 1.00 | 65.55 | A |
| 1888 | N | ASP | A | 1251 | −40.421 | 14.623 | 29.121 | 1.00 | 60.46 | A |
| 1889 | CA | ASP | A | 1251 | −40.084 | 14.843 | 27.709 | 1.00 | 58.35 | A |
| 1890 | CB | ASP | A | 1251 | −40.270 | 16.301 | 27.280 | 1.00 | 60.51 | A |
| 1891 | CG | ASP | A | 1251 | −40.210 | 17.326 | 28.567 | 1.00 | 64.08 | A |
| 1892 | OD1 | ASP | A | 1251 | −41.297 | 17.809 | 29.088 | 1.00 | 61.40 | A |
| 1893 | OD2 | ASP | A | 1251 | −39.079 | 17.516 | 29.075 | 1.00 | 59.92 | A |
| 1894 | C | ASP | A | 1251 | −38.657 | 14.492 | 27.418 | 1.00 | 56.03 | A |
| 1895 | O | ASP | A | 1251 | −38.437 | 13.482 | 26.604 | 1.00 | 55.36 | A |
| 1896 | N | GLU | A | 1252 | −37.705 | 15.227 | 28.029 | 1.00 | 50.40 | A |
| 1897 | CA | GLU | A | 1252 | −36.271 | 15.094 | 27.466 | 1.00 | 49.71 | A |
| 1898 | CB | GLU | A | 1252 | −35.586 | 16.437 | 27.295 | 1.00 | 47.66 | A |
| 1899 | CG | GLU | A | 1252 | −36.590 | 17.486 | 27.562 | 1.00 | 49.20 | A |
| 1900 | CD | GLU | A | 1252 | −36.045 | 18.725 | 27.334 | 1.00 | 42.01 | A |
| 1901 | OE1 | GLU | A | 1252 | −36.728 | 19.857 | 27.404 | 1.00 | 46.11 | A |
| 1902 | OE2 | GLU | A | 1252 | −34.862 | 18.508 | 27.121 | 1.00 | 39.77 | A |
| 1903 | C | GLU | A | 1252 | −35.274 | 14.206 | 28.224 | 1.00 | 49.28 | A |
| 1904 | O | GLU | A | 1252 | −34.032 | 14.388 | 28.167 | 1.00 | 48.41 | A |
| 1905 | N | LEU | A | 1253 | −35.820 | 13.256 | 28.940 | 1.00 | 49.59 | A |
| 1906 | CA | LEU | A | 1253 | −34.971 | 12.561 | 29.872 | 1.00 | 51.69 | A |
| 1907 | CB | LEU | A | 1253 | −35.545 | 12.495 | 31.381 | 1.00 | 52.95 | A |
| 1908 | CG | LEU | A | 1253 | −35.456 | 13.932 | 32.045 | 1.00 | 53.96 | A |
| 1909 | CD1 | LEU | A | 1253 | −35.475 | 13.943 | 33.599 | 1.00 | 52.42 | A |
| 1910 | CD2 | LEU | A | 1253 | −34.356 | 14.786 | 31.661 | 1.00 | 48.25 | A |
| 1911 | C | LEU | A | 1253 | −34.778 | 11.197 | 29.303 | 1.00 | 50.64 | A |
| 1912 | O | LEU | A | 1253 | −35.848 | 10.559 | 29.010 | 1.00 | 50.38 | A |
| 1913 | N | PHE | A | 1254 | −33.472 | 10.833 | 29.127 | 1.00 | 47.18 | A |
| 1914 | CA | PHE | A | 1254 | −33.082 | 9.524 | 28.929 | 1.00 | 46.70 | A |
| 1915 | CB | PHE | A | 1254 | −32.362 | 9.330 | 27.569 | 1.00 | 45.57 | A |
| 1916 | CG | PHE | A | 1254 | −32.813 | 10.263 | 26.572 | 1.00 | 45.71 | A |
| 1917 | CD1 | PHE | A | 1254 | −33.885 | 10.001 | 25.873 | 1.00 | 40.21 | A |
| 1918 | CD2 | PHE | A | 1254 | −32.133 | 11.479 | 26.354 | 1.00 | 45.22 | A |
| 1919 | CE1 | PHE | A | 1254 | −34.380 | 11.027 | 25.009 | 1.00 | 48.57 | A |
| 1920 | CE2 | PHE | A | 1254 | −32.638 | 12.459 | 25.596 | 1.00 | 43.99 | A |
| 1921 | CZ | PHE | A | 1254 | −33.796 | 12.222 | 24.897 | 1.00 | 43.85 | A |
| 1922 | C | PHE | A | 1254 | −32.351 | 8.814 | 30.097 | 1.00 | 48.18 | A |
| 1923 | O | PHE | A | 1254 | −31.446 | 9.385 | 30.716 | 1.00 | 48.46 | A |
| 1924 | N | ALA | A | 1255 | −32.671 | 7.521 | 30.258 | 1.00 | 48.73 | A |
| 1925 | CA | ALA | A | 1255 | −31.897 | 6.627 | 30.984 | 1.00 | 49.88 | A |
| 1926 | CB | ALA | A | 1255 | −32.767 | 5.770 | 31.862 | 1.00 | 49.04 | A |
| 1927 | C | ALA | A | 1255 | −31.077 | 5.715 | 30.042 | 1.00 | 51.35 | A |
| 1928 | O | ALA | A | 1255 | −31.669 | 4.966 | 29.372 | 1.00 | 55.33 | A |
| 1929 | N | LEU | A | 1256 | −29.725 | 5.673 | 30.108 | 1.00 | 50.85 | A |
| 1930 | CA | LEU | A | 1256 | −28.909 | 4.869 | 29.264 | 1.00 | 48.78 | A |
| 1931 | CB | LEU | A | 1256 | −27.585 | 5.642 | 28.920 | 1.00 | 49.57 | A |
| 1932 | CG | LEU | A | 1256 | −27.624 | 7.174 | 28.528 | 1.00 | 47.97 | A |
| 1933 | CD1 | LEU | A | 1256 | −26.270 | 7.965 | 28.454 | 1.00 | 45.30 | A |
| 1934 | CD2 | LEU | A | 1256 | −28.688 | 7.770 | 27.470 | 1.00 | 37.83 | A |
| 1935 | C | LEU | A | 1256 | −28.703 | 3.695 | 30.088 | 1.00 | 48.66 | A |
| 1936 | O | LEU | A | 1256 | −28.530 | 3.869 | 31.293 | 1.00 | 49.64 | A |
| 1937 | N | GLU | A | 1257 | −28.731 | 2.478 | 29.497 | 1.00 | 47.77 | A |
| 1938 | CA | GLU | A | 1257 | −28.631 | 1.223 | 30.321 | 1.00 | 47.36 | A |
| 1939 | CB | GLU | A | 1257 | −29.943 | 0.442 | 30.347 | 1.00 | 47.62 | A |
| 1940 | CG | GLU | A | 1257 | −31.167 | 1.167 | 29.661 | 1.00 | 49.14 | A |
| 1941 | CD | GLU | A | 1257 | −32.476 | 0.699 | 30.149 | 1.00 | 47.74 | A |
| 1942 | OE1 | GLU | A | 1257 | −32.670 | −0.465 | 29.961 | 1.00 | 49.70 | A |
| 1943 | OE2 | GLU | A | 1257 | −33.339 | 1.432 | 30.702 | 1.00 | 46.47 | A |
| 1944 | C | GLU | A | 1257 | −27.488 | 0.309 | 29.875 | 1.00 | 49.43 | A |
| 1945 | O | GLU | A | 1257 | −26.884 | 0.525 | 28.841 | 1.00 | 53.65 | A |
| 1946 | N | GLN | A | 1258 | −27.108 | −0.694 | 30.633 | 1.00 | 49.31 | A |
| 1947 | CA | GLN | A | 1258 | −25.981 | −1.444 | 30.216 | 1.00 | 48.50 | A |
| 1948 | CB | GLN | A | 1258 | −25.522 | −2.418 | 31.347 | 1.00 | 50.03 | A |
| 1949 | CG | GLN | A | 1258 | −24.485 | −2.001 | 32.557 | 1.00 | 52.07 | A |
| 1950 | CD | GLN | A | 1258 | −23.838 | −3.270 | 33.324 | 1.00 | 48.58 | A |
| 1951 | OE1 | GLN | A | 1258 | −24.578 | −4.238 | 33.681 | 1.00 | 50.22 | A |
| 1952 | NE2 | GLN | A | 1258 | −22.514 | −3.297 | 33.497 | 1.00 | 38.25 | A |
| 1953 | C | GLN | A | 1258 | −26.513 | −2.247 | 28.980 | 1.00 | 49.22 | A |
| 1954 | O | GLN | A | 1258 | −27.483 | −3.036 | 29.166 | 1.00 | 49.63 | A |
| 1955 | N | SER | A | 1259 | −25.896 | −2.104 | 27.758 | 1.00 | 46.91 | A |
| 1956 | CA | SER | A | 1259 | −26.009 | −3.126 | 26.712 | 1.00 | 44.38 | A |
| 1957 | CB | SER | A | 1259 | −25.301 | −2.638 | 25.506 | 1.00 | 46.28 | A |
| 1958 | OG | SER | A | 1259 | −25.463 | −3.426 | 24.306 | 1.00 | 47.23 | A |
| 1959 | C | SER | A | 1259 | −25.421 | −4.463 | 26.960 | 1.00 | 45.72 | A |
| 1960 | O | SER | A | 1259 | −24.173 | −4.575 | 26.899 | 1.00 | 50.32 | A |
| 1961 | N | CYS | A | 1260 | −26.213 | −5.522 | 27.035 | 1.00 | 46.58 | A |
| 1962 | CA | CYS | A | 1260 | −25.731 | −6.899 | 27.186 | 1.00 | 45.72 | A |
| 1963 | CB | CYS | A | 1260 | −26.799 | −7.664 | 27.942 | 1.00 | 49.84 | A |
| 1964 | SG | CYS | A | 1260 | −26.448 | −9.330 | 28.719 | 1.00 | 52.29 | A |
| 1965 | C | CYS | A | 1260 | −25.540 | −7.551 | 25.828 | 1.00 | 46.16 | A |
| 1966 | O | CYS | A | 1260 | −26.107 | −7.094 | 24.853 | 1.00 | 45.33 | A |
| 1967 | N | ALA | A | 1261 | −24.705 | −8.587 | 25.769 | 1.00 | 45.39 | A |
| 1968 | CA | ALA | A | 1261 | −24.237 | −9.096 | 24.517 | 1.00 | 47.61 | A |
| 1969 | CB | ALA | A | 1261 | −22.994 | −9.926 | 24.690 | 1.00 | 44.84 | A |
| 1970 | C | ALA | A | 1261 | −25.354 | −9.896 | 23.873 | 1.00 | 49.18 | A |
| 1971 | O | ALA | A | 1261 | −25.855 | −10.880 | 24.503 | 1.00 | 53.58 | A |
| 1972 | N | GLN | A | 1262 | −25.767 | −9.541 | 22.660 | 1.00 | 47.00 | A |
| 1973 | CA | GLN | A | 1262 | −26.883 | −10.265 | 22.114 | 1.00 | 46.65 | A |
| 1974 | CB | GLN | A | 1262 | −27.822 | −9.283 | 21.475 | 1.00 | 49.30 | A |
| 1975 | CG | GLN | A | 1262 | −29.227 | −9.358 | 21.784 | 1.00 | 50.78 | A |
| 1976 | CD | GLN | A | 1262 | −29.690 | −8.436 | 22.926 | 1.00 | 52.76 | A |
| 1977 | OE1 | GLN | A | 1262 | −29.530 | −7.257 | 22.843 | 1.00 | 51.91 | A |
| 1978 | NE2 | GLN | A | 1262 | −30.419 | −8.979 | 23.905 | 1.00 | 56.16 | A |
| 1979 | C | GLN | A | 1262 | −26.257 | −11.095 | 21.064 | 1.00 | 45.25 | A |
| 1980 | O | GLN | A | 1262 | −25.388 | −10.695 | 20.351 | 1.00 | 43.29 | A |
| 1981 | N | VAL | A | 1263 | −26.732 | −12.298 | 20.945 | 1.00 | 47.61 | A |
| 1982 | CA | VAL | A | 1263 | −26.071 | −13.461 | 20.128 | 1.00 | 44.24 | A |
| 1983 | CB | VAL | A | 1263 | −25.605 | −14.641 | 21.027 | 1.00 | 44.47 | A |
| 1984 | CG1 | VAL | A | 1263 | −24.227 | −14.701 | 21.109 | 1.00 | 47.46 | A |
| 1985 | CG2 | VAL | A | 1263 | −26.291 | −14.559 | 22.530 | 1.00 | 43.71 | A |
| 1986 | C | VAL | A | 1263 | −27.067 | −14.247 | 19.290 | 1.00 | 42.49 | A |
| 1987 | O | VAL | A | 1263 | −28.261 | −14.298 | 19.530 | 1.00 | 39.77 | A |
| 1988 | N | VAL | A | 1264 | −26.483 | −14.901 | 18.343 | 1.00 | 44.10 | A |
| 1989 | CA | VAL | A | 1264 | −27.122 | −15.748 | 17.416 | 1.00 | 44.97 | A |
| 1990 | CB | VAL | A | 1264 | −27.025 | −15.104 | 16.053 | 1.00 | 47.14 | A |
| 1991 | CG1 | VAL | A | 1264 | −26.757 | −16.204 | 14.772 | 1.00 | 50.51 | A |
| 1992 | CG2 | VAL | A | 1264 | −28.220 | −14.085 | 15.788 | 1.00 | 42.27 | A |
| 1993 | C | VAL | A | 1264 | −26.262 | −16.974 | 17.523 | 1.00 | 44.75 | A |
| 1994 | O | VAL | A | 1264 | −25.057 | −16.930 | 17.463 | 1.00 | 44.44 | A |
| 1995 | N | LEU | A | 1265 | −26.943 | −18.036 | 17.758 | 1.00 | 46.47 | A |
| 1996 | CA | LEU | A | 1265 | −26.483 | −19.350 | 17.964 | 1.00 | 49.28 | A |
| 1997 | CB | LEU | A | 1265 | −27.338 | −19.708 | 19.169 | 1.00 | 49.87 | A |
| 1998 | CG | LEU | A | 1265 | −26.719 | −19.143 | 20.459 | 1.00 | 44.76 | A |
| 1999 | CD1 | LEU | A | 1265 | −27.734 | −18.925 | 21.484 | 1.00 | 33.99 | A |
| 2000 | CD2 | LEU | A | 1265 | −25.795 | −20.371 | 20.849 | 1.00 | 38.97 | A |
| 2001 | C | LEU | A | 1265 | −26.852 | −20.294 | 16.771 | 1.00 | 51.57 | A |
| 2002 | O | LEU | A | 1265 | −28.075 | −20.415 | 16.330 | 1.00 | 52.77 | A |
| 2003 | N | GLN | A | 1266 | −25.857 | −20.965 | 16.218 | 1.00 | 49.83 | A |
| 2004 | CA | GLN | A | 1266 | −26.142 | −21.723 | 14.916 | 1.00 | 50.84 | A |
| 2005 | CB | GLN | A | 1266 | −25.126 | −21.470 | 13.709 | 1.00 | 51.55 | A |
| 2006 | CG | GLN | A | 1266 | −25.567 | −21.776 | 12.224 | 1.00 | 43.04 | A |
| 2007 | CD | GLN | A | 1266 | −24.451 | −21.298 | 11.244 | 1.00 | 43.42 | A |
| 2008 | OE1 | GLN | A | 1266 | −23.478 | −22.023 | 11.006 | 1.00 | 33.27 | A |
| 2009 | NE2 | GLN | A | 1266 | −24.496 | −20.082 | 10.817 | 1.00 | 31.22 | A |
| 2010 | C | GLN | A | 1266 | −25.931 | −23.106 | 15.248 | 1.00 | 52.93 | A |
| 2011 | O | GLN | A | 1266 | −25.051 | −23.405 | 16.174 | 1.00 | 52.88 | A |
| 2012 | N | ALA | A | 1267 | −26.578 | −23.951 | 14.422 | 1.00 | 52.65 | A |
| 2013 | CA | ALA | A | 1267 | −26.540 | −25.440 | 14.686 | 1.00 | 51.11 | A |
| 2014 | CB | ALA | A | 1267 | −27.813 | −25.982 | 14.474 | 1.00 | 50.66 | A |
| 2015 | C | ALA | A | 1267 | −25.556 | −26.050 | 13.822 | 1.00 | 49.58 | A |
| 2016 | O | ALA | A | 1267 | −24.943 | −25.339 | 13.045 | 1.00 | 51.99 | A |
| 2017 | N | ALA | A | 1268 | −25.261 | −27.308 | 13.999 | 1.00 | 50.87 | A |
| 2018 | CA | ALA | A | 1268 | −24.449 | −28.021 | 13.007 | 1.00 | 51.53 | A |
| 2019 | CB | ALA | A | 1268 | −24.154 | −29.313 | 13.388 | 1.00 | 49.16 | A |
| 2020 | C | ALA | A | 1268 | −25.123 | −28.001 | 11.607 | 1.00 | 54.82 | A |
| 2021 | O | ALA | A | 1268 | −24.395 | −27.764 | 10.579 | 1.00 | 57.07 | A |
| 2022 | N | ASN | A | 1269 | −26.466 | −28.225 | 11.551 | 1.00 | 55.26 | A |
| 2023 | CA | ASN | A | 1269 | −27.306 | −28.397 | 10.259 | 1.00 | 52.37 | A |
| 2024 | CB | ASN | A | 1269 | −28.804 | −28.622 | 10.544 | 1.00 | 51.64 | A |
| 2025 | CG | ASN | A | 1269 | −29.289 | −27.572 | 11.464 | 1.00 | 50.17 | A |
| 2026 | OD1 | ASN | A | 1269 | −28.419 | −26.834 | 11.845 | 1.00 | 59.58 | A |
| 2027 | ND2 | ASN | A | 1269 | −30.548 | −27.509 | 11.933 | 1.00 | 46.37 | A |
| 2028 | C | ASN | A | 1269 | −27.325 | −27.009 | 9.706 | 1.00 | 54.22 | A |
| 2029 | O | ASN | A | 1269 | −28.475 | −26.528 | 9.040 | 1.00 | 50.71 | A |
| 2030 | N | GLU | A | 1270 | −26.162 | −26.357 | 10.060 | 1.00 | 51.83 | A |
| 2031 | CA | GLU | A | 1270 | −25.859 | −24.997 | 9.653 | 1.00 | 54.53 | A |
| 2032 | CB | GLU | A | 1270 | −25.695 | −24.941 | 8.116 | 1.00 | 57.05 | A |
| 2033 | CG | GLU | A | 1270 | −24.839 | −26.041 | 7.497 | 1.00 | 62.14 | A |
| 2034 | CD | GLU | A | 1270 | −23.415 | −25.807 | 7.928 | 1.00 | 73.47 | A |
| 2035 | OE1 | GLU | A | 1270 | −22.607 | −25.239 | 7.103 | 1.00 | 76.14 | A |
| 2036 | OE2 | GLU | A | 1270 | −23.132 | −26.155 | 9.124 | 1.00 | 77.02 | A |
| 2037 | C | GLU | A | 1270 | −27.009 | −24.033 | 9.956 | 1.00 | 53.08 | A |
| 2038 | O | GLU | A | 1270 | −26.968 | −22.933 | 9.596 | 1.00 | 54.60 | A |
| 2039 | N | ARG | A | 1271 | −28.083 | −24.430 | 10.535 | 1.00 | 52.10 | A |
| 2040 | CA | ARG | A | 1271 | −29.111 | −23.471 | 10.611 | 1.00 | 53.73 | A |
| 2041 | CB | ARG | A | 1271 | −30.612 | −24.046 | 10.417 | 1.00 | 55.12 | A |
| 2042 | CG | ARG | A | 1271 | −31.073 | −24.660 | 9.026 | 1.00 | 55.75 | A |
| 2043 | CD | ARG | A | 1271 | −30.740 | −23.879 | 7.792 | 1.00 | 59.58 | A |
| 2044 | NE | ARG | A | 1271 | −31.770 | −22.918 | 7.427 | 1.00 | 56.92 | A |
| 2045 | CZ | ARG | A | 1271 | −33.026 | −23.276 | 7.426 | 1.00 | 55.62 | A |
| 2046 | NH1 | ARG | A | 1271 | −33.277 | −24.514 | 7.791 | 1.00 | 53.47 | A |
| 2047 | NH2 | ARG | A | 1271 | −33.999 | −22.395 | 7.135 | 1.00 | 56.51 | A |
| 2048 | C | ARG | A | 1271 | −28.968 | −22.822 | 11.984 | 1.00 | 53.94 | A |
| 2049 | O | ARG | A | 1271 | −28.190 | −23.263 | 12.857 | 1.00 | 53.73 | A |
| 2050 | N | ASN | A | 1272 | −29.797 | −21.781 | 12.125 | 1.00 | 53.81 | A |
| 2051 | CA | ASN | A | 1272 | −30.001 | −21.011 | 13.293 | 1.00 | 54.01 | A |
| 2052 | CB | ASN | A | 1272 | −30.014 | −19.595 | 12.794 | 1.00 | 55.69 | A |
| 2053 | CG | ASN | A | 1272 | −28.644 | −19.034 | 12.602 | 1.00 | 55.62 | A |
| 2054 | OD1 | ASN | A | 1272 | −27.575 | −19.741 | 12.551 | 1.00 | 62.85 | A |
| 2055 | ND2 | ASN | A | 1272 | −28.644 | −17.769 | 12.498 | 1.00 | 41.76 | A |
| 2056 | C | ASN | A | 1272 | −31.314 | −21.103 | 13.900 | 1.00 | 53.33 | A |
| 2057 | O | ASN | A | 1272 | −32.332 | −21.177 | 13.218 | 1.00 | 52.16 | A |
| 2058 | N | VAL | A | 1273 | −31.284 | −20.784 | 15.175 | 1.00 | 54.43 | A |
| 2059 | CA | VAL | A | 1273 | −32.428 | −21.025 | 16.080 | 1.00 | 54.62 | A |
| 2060 | CB | VAL | A | 1273 | −32.023 | −21.811 | 17.407 | 1.00 | 52.55 | A |
| 2061 | CG1 | VAL | A | 1273 | −30.618 | −22.040 | 17.453 | 1.00 | 49.49 | A |
| 2062 | CG2 | VAL | A | 1273 | −32.439 | −21.160 | 18.592 | 1.00 | 50.07 | A |
| 2063 | C | VAL | A | 1273 | −33.353 | −19.865 | 16.287 | 1.00 | 55.67 | A |
| 2064 | O | VAL | A | 1273 | −32.931 | −18.741 | 16.544 | 1.00 | 59.47 | A |
| 2065 | N | SER | A | 1274 | −34.635 | −20.160 | 16.266 | 1.00 | 55.14 | A |
| 2066 | CA | SER | A | 1274 | −35.661 | −19.172 | 16.182 | 1.00 | 52.55 | A |
| 2067 | CB | SER | A | 1274 | −36.405 | −19.248 | 14.815 | 1.00 | 52.54 | A |
| 2068 | OG | SER | A | 1274 | −36.965 | −17.954 | 14.430 | 1.00 | 55.62 | A |
| 2069 | C | SER | A | 1274 | −36.609 | −19.508 | 17.129 | 1.00 | 50.16 | A |
| 2070 | O | SER | A | 1274 | −36.705 | −20.594 | 17.516 | 1.00 | 49.36 | A |
| 2071 | N | GLY | A | 1275 | −37.284 | −18.481 | 17.547 | 1.00 | 53.13 | A |
| 2072 | CA | GLY | A | 1275 | −38.593 | −18.494 | 18.195 | 1.00 | 53.02 | A |
| 2073 | C | GLY | A | 1275 | −39.628 | −18.598 | 17.111 | 1.00 | 53.26 | A |
| 2074 | O | GLY | A | 1275 | −40.723 | −18.902 | 17.426 | 1.00 | 52.63 | A |
| 2075 | N | ARG | A | 1276 | −39.217 | −18.533 | 15.819 | 1.00 | 56.33 | A |
| 2076 | CA | ARG | A | 1276 | −40.124 | −18.546 | 14.618 | 1.00 | 57.25 | A |
| 2077 | CB | ARG | A | 1276 | −39.427 | −18.247 | 13.275 | 1.00 | 56.40 | A |
| 2078 | CG | ARG | A | 1276 | −39.843 | −17.004 | 12.567 | 1.00 | 52.36 | A |
| 2079 | CD | ARG | A | 1276 | −38.692 | −16.560 | 11.627 | 1.00 | 54.55 | A |
| 2080 | NE | ARG | A | 1276 | −38.479 | −15.112 | 11.283 | 1.00 | 46.53 | A |
| 2081 | CZ | ARG | A | 1276 | −37.266 | −14.682 | 10.912 | 1.00 | 46.62 | A |
| 2082 | NH1 | ARG | A | 1276 | −36.189 | −15.484 | 10.799 | 1.00 | 41.73 | A |
| 2083 | NH2 | ARG | A | 1276 | −37.068 | −13.427 | 10.782 | 1.00 | 48.05 | A |
| 2084 | C | ARG | A | 1276 | −40.697 | −19.894 | 14.480 | 1.00 | 60.22 | A |
| 2085 | O | ARG | A | 1276 | −39.948 | −20.831 | 14.476 | 1.00 | 60.07 | A |
| 2086 | N | GLN | A | 1277 | −42.012 | −19.989 | 14.242 | 1.00 | 63.04 | A |
| 2087 | CA | GLN | A | 1277 | −42.584 | −21.287 | 14.117 | 1.00 | 65.55 | A |
| 2088 | CB | GLN | A | 1277 | −41.896 | −22.137 | 13.025 | 1.00 | 64.18 | A |
| 2089 | CG | GLN | A | 1277 | −42.628 | −22.109 | 11.576 | 1.00 | 67.64 | A |
| 2090 | CD | GLN | A | 1277 | −44.213 | −21.898 | 11.594 | 1.00 | 63.83 | A |
| 2091 | OE1 | GLN | A | 1277 | −44.973 | −22.754 | 12.130 | 1.00 | 61.97 | A |
| 2092 | NE2 | GLN | A | 1277 | −44.671 | −20.752 | 11.047 | 1.00 | 48.65 | A |
| 2093 | C | GLN | A | 1277 | −42.405 | −21.831 | 15.553 | 1.00 | 67.86 | A |
| 2094 | O | GLN | A | 1277 | −41.829 | −22.920 | 15.749 | 1.00 | 68.82 | A |
| 2095 | N | THR | A | 1278 | −42.976 | −21.054 | 16.489 | 1.00 | 69.24 | A |
| 2096 | CA | THR | A | 1278 | −42.657 | −20.977 | 17.891 | 1.00 | 70.98 | A |
| 2097 | CB | THR | A | 1278 | −43.385 | −19.774 | 18.675 | 1.00 | 70.64 | A |
| 2098 | OG1 | THR | A | 1278 | −42.764 | −19.547 | 19.946 | 1.00 | 64.44 | A |
| 2099 | CG2 | THR | A | 1278 | −44.835 | −20.048 | 18.836 | 1.00 | 67.76 | A |
| 2100 | C | THR | A | 1278 | −42.880 | −22.215 | 18.701 | 1.00 | 74.06 | A |
| 2101 | O | THR | A | 1278 | −42.569 | −23.352 | 18.262 | 1.00 | 76.04 | A |
| 2102 | N | MET | A | 1279 | −43.345 | −22.011 | 19.939 | 1.00 | 74.15 | A |
| 2103 | CA | MET | A | 1279 | −43.349 | −23.158 | 20.808 | 1.00 | 73.96 | A |
| 2104 | CB | MET | A | 1279 | −43.710 | −24.451 | 19.989 | 1.00 | 71.91 | A |
| 2105 | CG | MET | A | 1279 | −44.639 | −25.455 | 20.658 | 1.00 | 70.67 | A |
| 2106 | SD | MET | A | 1279 | −45.796 | −24.779 | 21.915 | 1.00 | 64.86 | A |
| 2107 | CE | MET | A | 1279 | −46.733 | −26.264 | 22.135 | 1.00 | 60.46 | A |
| 2108 | C | MET | A | 1279 | −41.944 | −23.196 | 21.506 | 1.00 | 73.87 | A |
| 2109 | O | MET | A | 1279 | −41.695 | −22.328 | 22.406 | 1.00 | 74.09 | A |
| 2110 | N | ASP | A | 1280 | −41.081 | −24.173 | 21.127 | 1.00 | 71.79 | A |
| 2111 | CA | ASP | A | 1280 | −39.974 | −24.713 | 21.988 | 1.00 | 69.68 | A |
| 2112 | CB | ASP | A | 1280 | −39.983 | −26.287 | 22.152 | 1.00 | 69.86 | A |
| 2113 | CG | ASP | A | 1280 | −41.448 | −26.932 | 22.461 | 1.00 | 72.10 | A |
| 2114 | OD1 | ASP | A | 1280 | −42.394 | −26.757 | 21.632 | 1.00 | 75.65 | A |
| 2115 | OD2 | ASP | A | 1280 | −41.693 | −27.649 | 23.513 | 1.00 | 70.69 | A |
| 2116 | C | ASP | A | 1280 | −38.841 | −24.225 | 21.157 | 1.00 | 69.93 | A |
| 2117 | O | ASP | A | 1280 | −38.982 | −23.185 | 20.494 | 1.00 | 71.89 | A |
| 2118 | N | LEU | A | 1281 | −37.732 | −24.941 | 21.038 | 1.00 | 69.08 | A |
| 2119 | CA | LEU | A | 1281 | −36.681 | −24.364 | 20.180 | 1.00 | 66.54 | A |
| 2120 | CB | LEU | A | 1281 | −35.587 | −23.647 | 21.037 | 1.00 | 64.84 | A |
| 2121 | CG | LEU | A | 1281 | −35.981 | −22.350 | 21.746 | 1.00 | 65.58 | A |
| 2122 | CD1 | LEU | A | 1281 | −35.232 | −21.955 | 23.025 | 1.00 | 68.09 | A |
| 2123 | CD2 | LEU | A | 1281 | −35.863 | −21.269 | 20.846 | 1.00 | 65.17 | A |
| 2124 | C | LEU | A | 1281 | −36.099 | −25.301 | 19.048 | 1.00 | 65.78 | A |
| 2125 | O | LEU | A | 1281 | −35.750 | −26.458 | 19.234 | 1.00 | 64.93 | A |
| 2126 | N | SER | A | 1282 | −35.967 | −24.729 | 17.873 | 1.00 | 65.34 | A |
| 2127 | CA | SER | A | 1282 | −35.545 | −25.451 | 16.684 | 1.00 | 64.85 | A |
| 2128 | CB | SER | A | 1282 | −36.740 | −25.945 | 15.860 | 1.00 | 65.16 | A |
| 2129 | OG | SER | A | 1282 | −37.767 | −24.925 | 15.691 | 1.00 | 66.46 | A |
| 2130 | C | SER | A | 1282 | −34.781 | −24.423 | 15.852 | 1.00 | 63.77 | A |
| 2131 | O | SER | A | 1282 | −35.369 | −23.420 | 15.417 | 1.00 | 60.98 | A |
| 2132 | N | ALA | A | 1283 | −33.466 | −24.707 | 15.724 | 1.00 | 61.86 | A |
| 2133 | CA | ALA | A | 1283 | −32.610 | −24.151 | 14.749 | 1.00 | 59.12 | A |
| 2134 | CB | ALA | A | 1283 | −31.297 | −24.642 | 14.954 | 1.00 | 59.47 | A |
| 2135 | C | ALA | A | 1283 | −33.124 | −24.748 | 13.477 | 1.00 | 58.43 | A |
| 2136 | O | ALA | A | 1283 | −32.447 | −25.734 | 12.965 | 1.00 | 56.69 | A |
| 2137 | N | ASN | A | 1284 | −34.226 | −24.113 | 13.011 | 1.00 | 55.91 | A |
| 2138 | CA | ASN | A | 1284 | −34.880 | −24.195 | 11.681 | 1.00 | 57.17 | A |
| 2139 | CB | ASN | A | 1284 | −36.376 | −24.164 | 11.915 | 1.00 | 57.94 | A |
| 2140 | CG | ASN | A | 1284 | −36.714 | −23.124 | 12.906 | 1.00 | 57.22 | A |
| 2141 | OD1 | ASN | A | 1284 | −35.831 | −22.426 | 13.331 | 1.00 | 54.31 | A |
| 2142 | ND2 | ASN | A | 1284 | −37.915 | −23.092 | 13.369 | 1.00 | 57.18 | A |
| 2143 | C | ASN | A | 1284 | −34.621 | −23.037 | 10.688 | 1.00 | 57.75 | A |
| 2144 | O | ASN | A | 1284 | −34.134 | −23.296 | 9.579 | 1.00 | 60.33 | A |
| 2145 | N | GLN | A | 1285 | −35.044 | −21.802 | 10.984 | 1.00 | 57.48 | A |
| 2146 | CA | GLN | A | 1285 | −34.458 | −20.549 | 10.261 | 1.00 | 56.05 | A |
| 2147 | CB | GLN | A | 1285 | −34.932 | −19.164 | 10.761 | 1.00 | 54.06 | A |
| 2148 | CG | GLN | A | 1285 | −36.364 | −19.127 | 11.317 | 1.00 | 52.12 | A |
| 2149 | CD | GLN | A | 1285 | −37.331 | −19.696 | 10.379 | 1.00 | 45.75 | A |
| 2150 | OE1 | GLN | A | 1285 | −37.161 | −19.573 | 9.167 | 1.00 | 56.75 | A |
| 2151 | NE2 | GLN | A | 1285 | −38.416 | −20.162 | 10.880 | 1.00 | 44.35 | A |
| 2152 | C | GLN | A | 1285 | −32.991 | −20.456 | 10.079 | 1.00 | 54.52 | A |
| 2153 | O | GLN | A | 1285 | −32.251 | −21.191 | 10.644 | 1.00 | 55.07 | A |
| 2154 | N | ASP | A | 1286 | −32.617 | −19.468 | 9.278 | 1.00 | 55.60 | A |
| 2155 | CA | ASP | A | 1286 | −31.264 | −19.260 | 8.753 | 1.00 | 55.76 | A |
| 2156 | CB | ASP | A | 1286 | −30.860 | −20.208 | 7.601 | 1.00 | 56.69 | A |
| 2157 | CG | ASP | A | 1286 | −31.524 | −19.846 | 6.292 | 1.00 | 57.05 | A |
| 2158 | OD1 | ASP | A | 1286 | −30.762 | −19.594 | 5.332 | 1.00 | 51.80 | A |
| 2159 | OD2 | ASP | A | 1286 | −32.801 | −19.826 | 6.207 | 1.00 | 62.40 | A |
| 2160 | C | ASP | A | 1286 | −31.090 | −17.895 | 8.237 | 1.00 | 55.77 | A |
| 2161 | O | ASP | A | 1286 | −30.385 | −17.742 | 7.256 | 1.00 | 56.28 | A |
| 2162 | N | GLU | A | 1287 | −31.708 | −16.960 | 8.937 | 1.00 | 55.51 | A |
| 2163 | CA | GLU | A | 1287 | −31.438 | −15.516 | 9.009 | 1.00 | 57.62 | A |
| 2164 | CB | GLU | A | 1287 | −32.761 | −14.753 | 8.673 | 1.00 | 56.24 | A |
| 2165 | CG | GLU | A | 1287 | −33.027 | −14.812 | 7.237 | 1.00 | 63.46 | A |
| 2166 | CD | GLU | A | 1287 | −34.463 | −15.040 | 6.835 | 1.00 | 67.81 | A |
| 2167 | OE1 | GLU | A | 1287 | −34.735 | −16.170 | 6.266 | 1.00 | 62.06 | A |
| 2168 | OE2 | GLU | A | 1287 | −35.270 | −14.068 | 7.061 | 1.00 | 70.34 | A |
| 2169 | C | GLU | A | 1287 | −31.027 | −15.068 | 10.490 | 1.00 | 57.64 | A |
| 2170 | O | GLU | A | 1287 | −31.153 | −15.878 | 11.432 | 1.00 | 59.88 | A |
| 2171 | N | GLU | A | 1288 | −30.772 | −13.772 | 10.684 | 1.00 | 54.37 | A |
| 2172 | CA | GLU | A | 1288 | −30.322 | −13.205 | 11.902 | 1.00 | 54.07 | A |
| 2173 | CB | GLU | A | 1288 | −28.928 | −12.676 | 11.656 | 1.00 | 56.10 | A |
| 2174 | CG | GLU | A | 1288 | −27.838 | −13.348 | 12.506 | 1.00 | 56.86 | A |
| 2175 | CD | GLU | A | 1288 | −26.614 | −13.537 | 11.705 | 1.00 | 60.27 | A |
| 2176 | OE1 | GLU | A | 1288 | −25.727 | −12.640 | 11.908 | 1.00 | 66.32 | A |
| 2177 | OE2 | GLU | A | 1288 | −26.503 | −14.556 | 10.880 | 1.00 | 57.45 | A |
| 2178 | C | GLU | A | 1288 | −31.226 | −12.018 | 12.528 | 1.00 | 55.17 | A |
| 2179 | O | GLU | A | 1288 | −30.758 | −10.785 | 12.648 | 1.00 | 53.18 | A |
| 2180 | N | THR | A | 1289 | −32.490 | −12.394 | 12.909 | 1.00 | 53.19 | A |
| 2181 | CA | THR | A | 1289 | −33.515 | −11.409 | 13.238 | 1.00 | 52.05 | A |
| 2182 | CB | THR | A | 1289 | −34.813 | −11.718 | 12.568 | 1.00 | 52.81 | A |
| 2183 | OG1 | THR | A | 1289 | −35.339 | −12.935 | 13.227 | 1.00 | 55.08 | A |
| 2184 | CG2 | THR | A | 1289 | −34.572 | −11.758 | 11.078 | 1.00 | 44.93 | A |
| 2185 | C | THR | A | 1289 | −33.958 | −11.337 | 14.701 | 1.00 | 50.30 | A |
| 2186 | O | THR | A | 1289 | −33.628 | −12.166 | 15.517 | 1.00 | 49.64 | A |
| 2187 | N | ASP | A | 1290 | −34.733 | −10.331 | 14.997 | 1.00 | 47.83 | A |
| 2188 | CA | ASP | A | 1290 | −35.131 | −10.188 | 16.266 | 1.00 | 49.76 | A |
| 2189 | CB | ASP | A | 1290 | −36.273 | −9.130 | 16.297 | 1.00 | 48.10 | A |
| 2190 | CG | ASP | A | 1290 | −35.740 | −7.711 | 16.473 | 1.00 | 47.30 | A |
| 2191 | OD1 | ASP | A | 1290 | −36.268 | −6.895 | 17.202 | 1.00 | 50.24 | A |
| 2192 | OD2 | ASP | A | 1290 | −34.640 | −7.385 | 16.013 | 1.00 | 51.86 | A |
| 2193 | C | ASP | A | 1290 | −35.262 | −11.629 | 16.979 | 1.00 | 52.84 | A |
| 2194 | O | ASP | A | 1290 | −34.654 | −11.925 | 17.998 | 1.00 | 55.90 | A |
| 2195 | N | GLN | A | 1291 | −35.857 | −12.617 | 16.352 | 1.00 | 53.55 | A |
| 2196 | CA | GLN | A | 1291 | −35.950 | −13.867 | 17.016 | 1.00 | 50.92 | A |
| 2197 | CB | GLN | A | 1291 | −37.291 | −14.412 | 16.762 | 1.00 | 51.41 | A |
| 2198 | CG | GLN | A | 1291 | −38.256 | −13.339 | 16.373 | 1.00 | 51.45 | A |
| 2199 | CD | GLN | A | 1291 | −39.439 | −13.933 | 15.680 | 1.00 | 61.15 | A |
| 2200 | OE1 | GLN | A | 1291 | −40.576 | −13.846 | 16.148 | 1.00 | 63.10 | A |
| 2201 | NE2 | GLN | A | 1291 | −39.166 | −14.648 | 14.584 | 1.00 | 65.14 | A |
| 2202 | C | GLN | A | 1291 | −34.934 | −14.912 | 16.635 | 1.00 | 50.34 | A |
| 2203 | O | GLN | A | 1291 | −35.161 | −16.121 | 16.868 | 1.00 | 51.88 | A |
| 2204 | N | GLU | A | 1292 | −33.775 | −14.536 | 16.189 | 1.00 | 47.41 | A |
| 2205 | CA | GLU | A | 1292 | −32.675 | −15.466 | 16.326 | 1.00 | 47.17 | A |
| 2206 | CB | GLU | A | 1292 | −32.133 | −15.935 | 14.982 | 1.00 | 47.90 | A |
| 2207 | CG | GLU | A | 1292 | −33.083 | −16.675 | 14.013 | 1.00 | 52.12 | A |
| 2208 | CD | GLU | A | 1292 | −34.361 | −15.860 | 13.514 | 1.00 | 61.13 | A |
| 2209 | OE1 | GLU | A | 1292 | −35.514 | −16.460 | 13.685 | 1.00 | 52.07 | A |
| 2210 | OE2 | GLU | A | 1292 | −34.184 | −14.672 | 12.953 | 1.00 | 54.40 | A |
| 2211 | C | GLU | A | 1292 | −31.646 | −14.609 | 17.112 | 1.00 | 46.86 | A |
| 2212 | O | GLU | A | 1292 | −30.590 | −14.318 | 16.639 | 1.00 | 48.49 | A |
| 2213 | N | THR | A | 1293 | −32.018 | −14.029 | 18.231 | 1.00 | 44.59 | A |
| 2214 | CA | THR | A | 1293 | −31.106 | −13.007 | 18.754 | 1.00 | 42.53 | A |
| 2215 | CB | THR | A | 1293 | −31.301 | −11.651 | 18.074 | 1.00 | 42.24 | A |
| 2216 | OG1 | THR | A | 1293 | −30.591 | −11.713 | 16.853 | 1.00 | 44.50 | A |
| 2217 | CG2 | THR | A | 1293 | −30.855 | −10.499 | 18.908 | 1.00 | 33.85 | A |
| 2218 | C | THR | A | 1293 | −31.307 | −13.000 | 20.261 | 1.00 | 42.79 | A |
| 2219 | O | THR | A | 1293 | −32.409 | −12.769 | 20.646 | 1.00 | 39.63 | A |
| 2220 | N | PHE | A | 1294 | −30.270 | −13.358 | 21.063 | 1.00 | 41.49 | A |
| 2221 | CA | PHE | A | 1294 | −30.599 | −13.917 | 22.424 | 1.00 | 43.96 | A |
| 2222 | CB | PHE | A | 1294 | −30.448 | −15.506 | 22.575 | 1.00 | 43.77 | A |
| 2223 | CG | PHE | A | 1294 | −31.331 | −16.243 | 21.608 | 1.00 | 44.21 | A |
| 2224 | CD1 | PHE | A | 1294 | −30.835 | −16.738 | 20.399 | 1.00 | 35.52 | A |
| 2225 | CD2 | PHE | A | 1294 | −32.744 | −16.224 | 21.769 | 1.00 | 45.34 | A |
| 2226 | CE1 | PHE | A | 1294 | −31.771 | −17.263 | 19.425 | 1.00 | 36.32 | A |
| 2227 | CE2 | PHE | A | 1294 | −33.590 | −16.819 | 20.738 | 1.00 | 41.54 | A |
| 2228 | CZ | PHE | A | 1294 | −33.063 | −17.250 | 19.622 | 1.00 | 31.86 | A |
| 2229 | C | PHE | A | 1294 | −29.643 | −13.233 | 23.318 | 1.00 | 43.75 | A |
| 2230 | O | PHE | A | 1294 | −28.393 | −13.211 | 23.036 | 1.00 | 47.31 | A |
| 2231 | N | GLN | A | 1295 | −30.204 | −12.596 | 24.309 | 1.00 | 40.73 | A |
| 2232 | CA | GLN | A | 1295 | −29.450 | −11.802 | 25.167 | 1.00 | 42.12 | A |
| 2233 | CB | GLN | A | 1295 | −30.276 | −10.881 | 25.976 | 1.00 | 41.58 | A |
| 2234 | CG | GLN | A | 1295 | −29.363 | −9.930 | 26.744 | 1.00 | 44.53 | A |
| 2235 | CD | GLN | A | 1295 | −30.150 | −8.759 | 27.195 | 1.00 | 41.71 | A |
| 2236 | OE1 | GLN | A | 1295 | −30.154 | −7.720 | 26.537 | 1.00 | 42.91 | A |
| 2237 | NE2 | GLN | A | 1295 | −30.991 | −8.986 | 28.219 | 1.00 | 40.72 | A |
| 2238 | C | GLN | A | 1295 | −28.829 | −12.748 | 26.081 | 1.00 | 45.58 | A |
| 2239 | O | GLN | A | 1295 | −29.510 | −13.240 | 27.104 | 1.00 | 45.84 | A |
| 2240 | N | LEU | A | 1296 | −27.519 | −13.001 | 25.753 | 1.00 | 46.03 | A |
| 2241 | CA | LEU | A | 1296 | −26.681 | −13.921 | 26.570 | 1.00 | 46.37 | A |
| 2242 | CB | LEU | A | 1296 | −25.339 | −14.143 | 25.839 | 1.00 | 44.21 | A |
| 2243 | CG | LEU | A | 1296 | −24.200 | −14.913 | 26.483 | 1.00 | 42.90 | A |
| 2244 | CD1 | LEU | A | 1296 | −23.374 | −15.649 | 25.553 | 1.00 | 44.17 | A |
| 2245 | CD2 | LEU | A | 1296 | −23.233 | −14.144 | 27.293 | 1.00 | 41.35 | A |
| 2246 | C | LEU | A | 1296 | −26.504 | −13.285 | 28.054 | 1.00 | 46.39 | A |
| 2247 | O | LEU | A | 1296 | −25.514 | −12.507 | 28.331 | 1.00 | 43.57 | A |
| 2248 | N | GLU | A | 1297 | −27.457 | −13.581 | 28.935 | 1.00 | 45.35 | A |
| 2249 | CA | GLU | A | 1297 | −27.269 | −13.151 | 30.367 | 1.00 | 52.41 | A |
| 2250 | CB | GLU | A | 1297 | −28.580 | −13.158 | 31.227 | 1.00 | 51.95 | A |
| 2251 | CG | GLU | A | 1297 | −29.905 | −13.086 | 30.440 | 1.00 | 57.22 | A |
| 2252 | CD | GLU | A | 1297 | −30.944 | −12.307 | 31.147 | 1.00 | 57.00 | A |
| 2253 | OE1 | GLU | A | 1297 | −30.474 | −11.577 | 32.054 | 1.00 | 53.53 | A |
| 2254 | OE2 | GLU | A | 1297 | −32.163 | −12.480 | 30.820 | 1.00 | 53.79 | A |
| 2255 | C | GLU | A | 1297 | −26.251 | −13.959 | 31.187 | 1.00 | 53.02 | A |
| 2256 | O | GLU | A | 1297 | −26.619 | −14.985 | 31.640 | 1.00 | 56.17 | A |
| 2257 | N | ILE | A | 1298 | −25.037 | −13.542 | 31.397 | 1.00 | 53.74 | A |
| 2258 | CA | ILE | A | 1298 | −24.100 | −14.351 | 32.279 | 1.00 | 55.23 | A |
| 2259 | CB | ILE | A | 1298 | −22.673 | −14.519 | 31.659 | 1.00 | 52.61 | A |
| 2260 | CG2 | ILE | A | 1298 | −21.885 | −15.349 | 32.517 | 1.00 | 53.99 | A |
| 2261 | CG1 | ILE | A | 1298 | −22.686 | −15.486 | 30.488 | 1.00 | 56.27 | A |
| 2262 | CD1 | ILE | A | 1298 | −21.529 | −15.375 | 29.439 | 1.00 | 52.33 | A |
| 2263 | C | ILE | A | 1298 | −23.913 | −13.825 | 33.752 | 1.00 | 54.63 | A |
| 2264 | O | ILE | A | 1298 | −22.770 | −13.582 | 34.149 | 1.00 | 55.67 | A |
| 2265 | N | ASP | A | 1299 | −24.992 | −13.540 | 34.460 | 1.00 | 56.07 | A |
| 2266 | CA | ASP | A | 1299 | −25.043 | −13.267 | 35.969 | 1.00 | 60.33 | A |
| 2267 | CB | ASP | A | 1299 | −26.221 | −14.008 | 36.642 | 1.00 | 62.12 | A |
| 2268 | CG | ASP | A | 1299 | −25.752 | −15.365 | 37.297 | 1.00 | 62.62 | A |
| 2269 | OD1 | ASP | A | 1299 | −25.062 | −16.194 | 36.605 | 1.00 | 63.43 | A |
| 2270 | OD2 | ASP | A | 1299 | −26.066 | −15.571 | 38.506 | 1.00 | 60.45 | A |
| 2271 | C | ASP | A | 1299 | −23.849 | −13.573 | 36.928 | 1.00 | 61.96 | A |
| 2272 | O | ASP | A | 1299 | −23.051 | −14.656 | 36.907 | 1.00 | 61.35 | A |
| 2273 | N | ARG | A | 1300 | −23.789 | −12.645 | 37.849 | 1.00 | 63.14 | A |
| 2274 | CA | ARG | A | 1300 | −22.528 | −12.577 | 38.575 | 1.00 | 63.13 | A |
| 2275 | CB | ARG | A | 1300 | −22.409 | −11.204 | 39.274 | 1.00 | 65.44 | A |
| 2276 | CG | ARG | A | 1300 | −21.945 | −10.025 | 38.206 | 1.00 | 62.76 | A |
| 2277 | CD | ARG | A | 1300 | −22.535 | −8.592 | 38.351 | 1.00 | 54.72 | A |
| 2278 | NE | ARG | A | 1300 | −23.884 | −8.722 | 38.904 | 1.00 | 50.16 | A |
| 2279 | CZ | ARG | A | 1300 | −24.799 | −7.745 | 39.025 | 1.00 | 48.12 | A |
| 2280 | NH1 | ARG | A | 1300 | −24.607 | −6.445 | 38.667 | 1.00 | 39.06 | A |
| 2281 | NH2 | ARG | A | 1300 | −25.900 | −8.138 | 39.530 | 1.00 | 48.47 | A |
| 2282 | C | ARG | A | 1300 | −22.237 | −13.911 | 39.372 | 1.00 | 63.91 | A |
| 2283 | O | ARG | A | 1300 | −21.129 | −14.508 | 39.211 | 1.00 | 61.70 | A |
| 2284 | N | ASP | A | 1301 | −23.289 | −14.421 | 40.040 | 1.00 | 63.49 | A |
| 2285 | CA | ASP | A | 1301 | −23.254 | −15.663 | 40.878 | 1.00 | 63.08 | A |
| 2286 | CB | ASP | A | 1301 | −24.625 | −16.135 | 41.531 | 1.00 | 62.65 | A |
| 2287 | CG | ASP | A | 1301 | −25.772 | −15.027 | 41.605 | 1.00 | 65.06 | A |
| 2288 | OD1 | ASP | A | 1301 | −26.990 | −15.538 | 41.803 | 1.00 | 64.87 | A |
| 2289 | OD2 | ASP | A | 1301 | −25.465 | −13.741 | 41.461 | 1.00 | 56.19 | A |
| 2290 | C | ASP | A | 1301 | −22.812 | −16.873 | 40.088 | 1.00 | 62.74 | A |
| 2291 | O | ASP | A | 1301 | −21.649 | −16.905 | 39.665 | 1.00 | 61.99 | A |
| 2292 | N | THR | A | 1302 | −23.737 | −17.891 | 40.081 | 1.00 | 62.04 | A |
| 2293 | CA | THR | A | 1302 | −23.772 | −19.140 | 39.351 | 1.00 | 60.33 | A |
| 2294 | CB | THR | A | 1302 | −25.244 | −19.400 | 38.572 | 1.00 | 62.02 | A |
| 2295 | OG1 | THR | A | 1302 | −25.278 | −18.828 | 37.267 | 1.00 | 60.51 | A |
| 2296 | CG2 | THR | A | 1302 | −26.532 | −18.938 | 39.309 | 1.00 | 62.03 | A |
| 2297 | C | THR | A | 1302 | −22.759 | −19.119 | 38.253 | 1.00 | 61.08 | A |
| 2298 | O | THR | A | 1302 | −22.071 | −20.214 | 37.930 | 1.00 | 57.20 | A |
| 2299 | N | LYS | A | 1303 | −22.761 | −17.882 | 37.623 | 1.00 | 61.88 | A |
| 2300 | CA | LYS | A | 1303 | −21.915 | −17.489 | 36.457 | 1.00 | 61.60 | A |
| 2301 | CB | LYS | A | 1303 | −20.461 | −17.961 | 36.748 | 1.00 | 61.48 | A |
| 2302 | CG | LYS | A | 1303 | −19.159 | −16.992 | 36.765 | 1.00 | 61.02 | A |
| 2303 | CD | LYS | A | 1303 | −19.175 | −15.873 | 37.942 | 1.00 | 57.72 | A |
| 2304 | CE | LYS | A | 1303 | −17.802 | −15.283 | 38.045 | 1.00 | 57.92 | A |
| 2305 | NZ | LYS | A | 1303 | −17.776 | −13.840 | 37.844 | 1.00 | 61.12 | A |
| 2306 | C | LYS | A | 1303 | −22.491 | −18.368 | 35.289 | 1.00 | 62.85 | A |
| 2307 | O | LYS | A | 1303 | −21.759 | −18.698 | 34.362 | 1.00 | 62.28 | A |
| 2308 | N | LYS | A | 1304 | −23.788 | −18.751 | 35.351 | 1.00 | 62.95 | A |
| 2309 | CA | LYS | A | 1304 | −24.346 | −19.688 | 34.406 | 1.00 | 64.20 | A |
| 2310 | CB | LYS | A | 1304 | −25.439 | −20.581 | 35.034 | 1.00 | 66.25 | A |
| 2311 | CG | LYS | A | 1304 | −25.023 | −22.019 | 35.625 | 1.00 | 64.38 | A |
| 2312 | CD | LYS | A | 1304 | −26.041 | −22.450 | 36.735 | 1.00 | 65.86 | A |
| 2313 | CE | LYS | A | 1304 | −26.317 | −24.030 | 36.786 | 1.00 | 66.63 | A |
| 2314 | NZ | LYS | A | 1304 | −25.048 | −24.859 | 36.863 | 1.00 | 65.05 | A |
| 2315 | C | LYS | A | 1304 | −24.755 | −18.854 | 33.166 | 1.00 | 64.10 | A |
| 2316 | O | LYS | A | 1304 | −24.168 | −17.813 | 33.016 | 1.00 | 66.49 | A |
| 2317 | N | CYS | A | 1305 | −25.548 | −19.322 | 32.198 | 1.00 | 61.89 | A |
| 2318 | CA | CYS | A | 1305 | −25.883 | −18.471 | 31.092 | 1.00 | 59.45 | A |
| 2319 | CB | CYS | A | 1305 | −25.130 | −18.845 | 29.883 | 1.00 | 59.40 | A |
| 2320 | SG | CYS | A | 1305 | −25.649 | −17.757 | 28.366 | 1.00 | 60.99 | A |
| 2321 | C | CYS | A | 1305 | −27.369 | −18.576 | 30.785 | 1.00 | 59.73 | A |
| 2322 | O | CYS | A | 1305 | −27.884 | −19.747 | 30.599 | 1.00 | 60.28 | A |
| 2323 | N | ALA | A | 1306 | −28.080 | −17.428 | 30.742 | 1.00 | 56.64 | A |
| 2324 | CA | ALA | A | 1306 | −29.483 | −17.550 | 30.632 | 1.00 | 55.01 | A |
| 2325 | CB | ALA | A | 1306 | −30.142 | −17.174 | 31.880 | 1.00 | 54.82 | A |
| 2326 | C | ALA | A | 1306 | −30.199 | −16.929 | 29.464 | 1.00 | 54.76 | A |
| 2327 | O | ALA | A | 1306 | −31.073 | −16.138 | 29.707 | 1.00 | 57.78 | A |
| 2328 | N | PHE | A | 1307 | −29.943 | −17.329 | 28.226 | 1.00 | 51.55 | A |
| 2329 | CA | PHE | A | 1307 | −30.506 | −16.597 | 27.096 | 1.00 | 50.57 | A |
| 2330 | CB | PHE | A | 1307 | −30.454 | −17.490 | 25.859 | 1.00 | 48.35 | A |
| 2331 | CG | PHE | A | 1307 | −29.040 | −18.104 | 25.604 | 1.00 | 44.69 | A |
| 2332 | CD1 | PHE | A | 1307 | −28.819 | −19.380 | 25.732 | 1.00 | 30.80 | A |
| 2333 | CD2 | PHE | A | 1307 | −27.973 | −17.349 | 25.181 | 1.00 | 44.28 | A |
| 2334 | CE1 | PHE | A | 1307 | −27.639 | −19.864 | 25.385 | 1.00 | 38.00 | A |
| 2335 | CE2 | PHE | A | 1307 | −26.760 | −17.857 | 24.931 | 1.00 | 37.31 | A |
| 2336 | CZ | PHE | A | 1307 | −26.572 | −19.105 | 25.003 | 1.00 | 35.33 | A |
| 2337 | C | PHE | A | 1307 | −31.919 | −15.896 | 27.272 | 1.00 | 49.78 | A |
| 2338 | O | PHE | A | 1307 | −32.832 | −16.606 | 27.675 | 1.00 | 53.55 | A |
| 2339 | N | ARG | A | 1308 | −32.092 | −14.575 | 27.051 | 1.00 | 45.29 | A |
| 2340 | CA | ARG | A | 1308 | −33.387 | −13.972 | 27.132 | 1.00 | 45.96 | A |
| 2341 | CB | ARG | A | 1308 | −33.272 | −12.511 | 27.487 | 1.00 | 46.09 | A |
| 2342 | CG | ARG | A | 1308 | −34.006 | −11.885 | 28.709 | 1.00 | 38.83 | A |
| 2343 | CD | ARG | A | 1308 | −35.390 | −12.015 | 28.519 | 1.00 | 38.95 | A |
| 2344 | NE | ARG | A | 1308 | −35.947 | −10.887 | 29.264 | 1.00 | 45.19 | A |
| 2345 | CZ | ARG | A | 1308 | −35.449 | −9.658 | 29.230 | 1.00 | 48.02 | A |
| 2346 | NH1 | ARG | A | 1308 | −34.530 | −9.364 | 28.360 | 1.00 | 48.04 | A |
| 2347 | NH2 | ARG | A | 1308 | −35.990 | −8.674 | 29.943 | 1.00 | 53.38 | A |
| 2348 | C | ARG | A | 1308 | −33.888 | −13.926 | 25.742 | 1.00 | 46.75 | A |
| 2349 | O | ARG | A | 1308 | −33.116 | −13.783 | 24.923 | 1.00 | 47.35 | A |
| 2350 | N | THR | A | 1309 | −35.194 | −13.944 | 25.446 | 1.00 | 48.50 | A |
| 2351 | CA | THR | A | 1309 | −35.782 | −13.836 | 24.023 | 1.00 | 47.11 | A |
| 2352 | CB | THR | A | 1309 | −36.903 | −14.811 | 23.938 | 1.00 | 45.73 | A |
| 2353 | OG1 | THR | A | 1309 | −37.894 | −14.369 | 24.912 | 1.00 | 48.67 | A |
| 2354 | CG2 | THR | A | 1309 | −36.495 | −16.233 | 24.359 | 1.00 | 40.34 | A |
| 2355 | C | THR | A | 1309 | −36.399 | −12.306 | 23.903 | 1.00 | 47.28 | A |
| 2356 | O | THR | A | 1309 | −36.668 | −11.666 | 24.968 | 1.00 | 47.89 | A |
| 2357 | N | HIS | A | 1310 | −36.606 | −11.728 | 22.701 | 1.00 | 45.42 | A |
| 2358 | CA | HIS | A | 1310 | −37.246 | −10.416 | 22.569 | 1.00 | 46.98 | A |
| 2359 | CB | HIS | A | 1310 | −37.336 | −9.953 | 21.146 | 1.00 | 45.07 | A |
| 2360 | CG | HIS | A | 1310 | −38.080 | −10.909 | 20.289 | 1.00 | 46.29 | A |
| 2361 | CD2 | HIS | A | 1310 | −37.719 | −12.141 | 19.840 | 1.00 | 47.63 | A |
| 2362 | ND1 | HIS | A | 1310 | −39.409 | −10.711 | 19.893 | 1.00 | 49.66 | A |
| 2363 | CE1 | HIS | A | 1310 | −39.835 | −11.786 | 19.224 | 1.00 | 46.35 | A |
| 2364 | NE2 | HIS | A | 1310 | −38.845 | −12.683 | 19.229 | 1.00 | 54.25 | A |
| 2365 | C | HIS | A | 1310 | −38.663 | −10.431 | 23.049 | 1.00 | 52.14 | A |
| 2366 | O | HIS | A | 1310 | −39.159 | −9.336 | 23.489 | 1.00 | 57.70 | A |
| 2367 | N | THR | A | 1311 | −39.413 | −11.555 | 22.991 | 1.00 | 53.67 | A |
| 2368 | CA | THR | A | 1311 | −40.762 | −11.508 | 23.610 | 1.00 | 52.16 | A |
| 2369 | CB | THR | A | 1311 | −41.300 | −12.936 | 23.433 | 1.00 | 54.44 | A |
| 2370 | OG1 | THR | A | 1311 | −41.387 | −13.596 | 24.751 | 1.00 | 54.84 | A |
| 2371 | CG2 | THR | A | 1311 | −40.432 | −13.716 | 22.519 | 1.00 | 40.18 | A |
| 2372 | C | THR | A | 1311 | −40.638 | −11.294 | 25.225 | 1.00 | 52.76 | A |
| 2373 | O | THR | A | 1311 | −41.574 | −11.335 | 25.966 | 1.00 | 51.73 | A |
| 2374 | N | GLY | A | 1312 | −39.430 | −11.200 | 25.767 | 1.00 | 53.22 | A |
| 2375 | CA | GLY | A | 1312 | −39.245 | −11.201 | 27.232 | 1.00 | 53.65 | A |
| 2376 | C | GLY | A | 1312 | −39.285 | −12.557 | 28.000 | 1.00 | 55.17 | A |
| 2377 | O | GLY | A | 1312 | −39.739 | −12.545 | 29.162 | 1.00 | 56.16 | A |
| 2378 | N | LYS | A | 1313 | −38.889 | −13.714 | 27.364 | 1.00 | 52.15 | A |
| 2379 | CA | LYS | A | 1313 | −39.025 | −15.073 | 27.995 | 1.00 | 48.68 | A |
| 2380 | CB | LYS | A | 1313 | −40.026 | −15.963 | 27.399 | 1.00 | 44.37 | A |
| 2381 | CG | LYS | A | 1313 | −41.329 | −15.720 | 28.042 | 1.00 | 41.73 | A |
| 2382 | CD | LYS | A | 1313 | −41.800 | −14.245 | 27.801 | 1.00 | 37.92 | A |
| 2383 | CE | LYS | A | 1313 | −42.938 | −13.729 | 28.699 | 1.00 | 36.62 | A |
| 2384 | NZ | LYS | A | 1313 | −44.013 | −14.677 | 28.388 | 1.00 | 36.38 | A |
| 2385 | C | LYS | A | 1313 | −37.702 | −15.624 | 27.850 | 1.00 | 49.51 | A |
| 2386 | O | LYS | A | 1313 | −36.832 | −14.902 | 27.260 | 1.00 | 51.86 | A |
| 2387 | N | TYR | A | 1314 | −37.482 | −16.822 | 28.416 | 1.00 | 47.74 | A |
| 2388 | CA | TYR | A | 1314 | −36.169 | −17.322 | 28.677 | 1.00 | 47.12 | A |
| 2389 | CB | TYR | A | 1314 | −36.104 | −17.397 | 30.241 | 1.00 | 51.07 | A |
| 2390 | CG | TYR | A | 1314 | −35.644 | −16.104 | 30.800 | 1.00 | 54.48 | A |
| 2391 | CD1 | TYR | A | 1314 | −36.579 | −15.041 | 31.039 | 1.00 | 56.56 | A |
| 2392 | CE1 | TYR | A | 1314 | −36.102 | −13.752 | 31.537 | 1.00 | 54.73 | A |
| 2393 | CD2 | TYR | A | 1314 | −34.211 | −15.834 | 30.937 | 1.00 | 54.49 | A |
| 2394 | CE2 | TYR | A | 1314 | −33.743 | −14.524 | 31.385 | 1.00 | 49.88 | A |
| 2395 | CZ | TYR | A | 1314 | −34.679 | −13.534 | 31.727 | 1.00 | 50.77 | A |
| 2396 | OH | TYR | A | 1314 | −34.216 | −12.300 | 32.128 | 1.00 | 48.25 | A |
| 2397 | C | TYR | A | 1314 | −36.050 | −18.669 | 28.113 | 1.00 | 46.35 | A |
| 2398 | O | TYR | A | 1314 | −37.071 | −19.396 | 27.987 | 1.00 | 47.04 | A |
| 2399 | N | TRP | A | 1315 | −34.845 | −19.066 | 27.708 | 1.00 | 45.60 | A |
| 2400 | CA | TRP | A | 1315 | −34.715 | −20.496 | 27.297 | 1.00 | 45.58 | A |
| 2401 | CB | TRP | A | 1315 | −33.423 | −20.857 | 26.670 | 1.00 | 40.95 | A |
| 2402 | CG | TRP | A | 1315 | −33.174 | −20.302 | 25.469 | 1.00 | 38.61 | A |
| 2403 | CD2 | TRP | A | 1315 | −32.311 | −20.871 | 24.444 | 1.00 | 34.25 | A |
| 2404 | CE2 | TRP | A | 1315 | −32.379 | −20.065 | 23.354 | 1.00 | 33.27 | A |
| 2405 | CE3 | TRP | A | 1315 | −31.381 | −21.883 | 24.432 | 1.00 | 33.32 | A |
| 2406 | CD1 | TRP | A | 1315 | −33.728 | −19.187 | 24.944 | 1.00 | 33.92 | A |
| 2407 | NE1 | TRP | A | 1315 | −33.242 | −19.024 | 23.658 | 1.00 | 33.24 | A |
| 2408 | CZ2 | TRP | A | 1315 | −31.430 | −20.207 | 22.250 | 1.00 | 37.03 | A |
| 2409 | CZ3 | TRP | A | 1315 | −30.623 | −22.144 | 23.292 | 1.00 | 31.61 | A |
| 2410 | CH2 | TRP | A | 1315 | −30.675 | −21.271 | 22.178 | 1.00 | 30.47 | A |
| 2411 | C | TRP | A | 1315 | −34.804 | −21.347 | 28.597 | 1.00 | 49.38 | A |
| 2412 | O | TRP | A | 1315 | −33.948 | −21.188 | 29.539 | 1.00 | 47.10 | A |
| 2413 | N | THR | A | 1316 | −35.862 | −22.195 | 28.586 | 1.00 | 52.15 | A |
| 2414 | CA | THR | A | 1316 | −36.332 | −23.035 | 29.755 | 1.00 | 53.79 | A |
| 2415 | CB | THR | A | 1316 | −37.843 | −22.930 | 30.052 | 1.00 | 51.85 | A |
| 2416 | OG1 | THR | A | 1316 | −38.156 | −21.581 | 30.401 | 1.00 | 55.16 | A |
| 2417 | CG2 | THR | A | 1316 | −38.103 | −23.600 | 31.269 | 1.00 | 54.88 | A |
| 2418 | C | THR | A | 1316 | −35.973 | −24.498 | 29.491 | 1.00 | 53.04 | A |
| 2419 | O | THR | A | 1316 | −35.158 | −24.737 | 28.580 | 1.00 | 52.01 | A |
| 2420 | N | LEU | A | 1317 | −36.472 | −25.425 | 30.330 | 1.00 | 53.12 | A |
| 2421 | CA | LEU | A | 1317 | −36.255 | −26.864 | 30.080 | 1.00 | 55.15 | A |
| 2422 | CB | LEU | A | 1317 | −34.977 | −27.435 | 30.749 | 1.00 | 58.03 | A |
| 2423 | CG | LEU | A | 1317 | −34.627 | −28.898 | 31.021 | 1.00 | 55.21 | A |
| 2424 | CD1 | LEU | A | 1317 | −33.443 | −28.802 | 31.949 | 1.00 | 46.05 | A |
| 2425 | CD2 | LEU | A | 1317 | −35.915 | −29.571 | 31.723 | 1.00 | 58.28 | A |
| 2426 | C | LEU | A | 1317 | −37.451 | −27.645 | 30.366 | 1.00 | 54.46 | A |
| 2427 | O | LEU | A | 1317 | −38.180 | −27.342 | 31.227 | 1.00 | 54.11 | A |
| 2428 | N | THR | A | 1318 | −37.687 | −28.584 | 29.489 | 1.00 | 57.29 | A |
| 2429 | CA | THR | A | 1318 | −38.900 | −29.373 | 29.476 | 1.00 | 57.17 | A |
| 2430 | CB | THR | A | 1318 | −39.554 | −29.522 | 28.020 | 1.00 | 57.20 | A |
| 2431 | OG1 | THR | A | 1318 | −38.549 | −29.503 | 27.013 | 1.00 | 54.08 | A |
| 2432 | CG2 | THR | A | 1318 | −40.539 | −28.472 | 27.704 | 1.00 | 53.93 | A |
| 2433 | C | THR | A | 1318 | −38.391 | −30.726 | 29.938 | 1.00 | 57.85 | A |
| 2434 | O | THR | A | 1318 | −37.243 | −31.135 | 29.692 | 1.00 | 58.03 | A |
| 2435 | N | ALA | A | 1319 | −39.291 | −31.417 | 30.599 | 1.00 | 59.71 | A |
| 2436 | CA | ALA | A | 1319 | −39.134 | −32.818 | 30.956 | 1.00 | 59.65 | A |
| 2437 | CB | ALA | A | 1319 | −40.238 | −33.227 | 31.550 | 1.00 | 59.23 | A |
| 2438 | C | ALA | A | 1319 | −38.827 | −33.742 | 29.862 | 1.00 | 60.09 | A |
| 2439 | O | ALA | A | 1319 | −38.223 | −34.684 | 30.131 | 1.00 | 63.86 | A |
| 2440 | N | THR | A | 1320 | −39.178 | −33.526 | 28.616 | 1.00 | 62.14 | A |
| 2441 | CA | THR | A | 1320 | −38.541 | −34.365 | 27.512 | 1.00 | 62.88 | A |
| 2442 | CB | THR | A | 1320 | −39.282 | −34.245 | 26.122 | 1.00 | 62.06 | A |
| 2443 | OG1 | THR | A | 1320 | −40.659 | −34.394 | 26.380 | 1.00 | 59.63 | A |
| 2444 | CG2 | THR | A | 1320 | −38.861 | −35.273 | 25.111 | 1.00 | 59.74 | A |
| 2445 | C | THR | A | 1320 | −37.055 | −34.122 | 27.378 | 1.00 | 63.36 | A |
| 2446 | O | THR | A | 1320 | −36.258 | −34.992 | 27.535 | 1.00 | 64.27 | A |
| 2447 | N | GLY | A | 1321 | −36.726 | −32.882 | 27.184 | 1.00 | 66.19 | A |
| 2448 | CA | GLY | A | 1321 | −35.375 | −32.371 | 27.159 | 1.00 | 67.99 | A |
| 2449 | C | GLY | A | 1321 | −35.571 | −31.355 | 26.068 | 1.00 | 69.77 | A |
| 2450 | O | GLY | A | 1321 | −34.750 | −31.246 | 25.150 | 1.00 | 71.91 | A |
| 2451 | N | GLY | A | 1322 | −36.698 | −30.629 | 26.139 | 1.00 | 70.04 | A |
| 2452 | CA | GLY | A | 1322 | −36.988 | −29.637 | 25.168 | 1.00 | 68.10 | A |
| 2453 | C | GLY | A | 1322 | −36.614 | −28.299 | 25.767 | 1.00 | 68.18 | A |
| 2454 | O | GLY | A | 1322 | −37.044 | −27.918 | 26.860 | 1.00 | 68.03 | A |
| 2455 | N | VAL | A | 1323 | −35.804 | −27.587 | 24.995 | 1.00 | 67.70 | A |
| 2456 | CA | VAL | A | 1323 | −35.547 | −26.221 | 25.211 | 1.00 | 65.63 | A |
| 2457 | CB | VAL | A | 1323 | −34.137 | −25.834 | 24.749 | 1.00 | 65.17 | A |
| 2458 | CG1 | VAL | A | 1323 | −33.788 | −24.449 | 25.342 | 1.00 | 69.02 | A |
| 2459 | CG2 | VAL | A | 1323 | −33.170 | −26.727 | 25.300 | 1.00 | 57.55 | A |
| 2460 | C | VAL | A | 1323 | −36.688 | −25.516 | 24.550 | 1.00 | 65.49 | A |
| 2461 | O | VAL | A | 1323 | −36.991 | −25.657 | 23.361 | 1.00 | 62.76 | A |
| 2462 | N | GLN | A | 1324 | −37.427 | −24.879 | 25.418 | 1.00 | 66.89 | A |
| 2463 | CA | GLN | A | 1324 | −38.676 | −24.224 | 24.967 | 1.00 | 68.49 | A |
| 2464 | CB | GLN | A | 1324 | −39.954 | −24.887 | 25.557 | 1.00 | 68.64 | A |
| 2465 | CG | GLN | A | 1324 | −40.316 | −24.496 | 27.109 | 1.00 | 73.20 | A |
| 2466 | CD | GLN | A | 1324 | −41.673 | −25.137 | 27.589 | 1.00 | 71.90 | A |
| 2467 | OE1 | GLN | A | 1324 | −42.144 | −24.892 | 28.747 | 1.00 | 75.14 | A |
| 2468 | NE2 | GLN | A | 1324 | −42.273 | −25.984 | 26.705 | 1.00 | 67.97 | A |
| 2469 | C | GLN | A | 1324 | −38.586 | −22.782 | 25.325 | 1.00 | 66.93 | A |
| 2470 | O | GLN | A | 1324 | −37.755 | −22.370 | 26.146 | 1.00 | 69.80 | A |
| 2471 | N | SER | A | 1325 | −39.354 | −21.950 | 24.692 | 1.00 | 66.19 | A |
| 2472 | CA | SER | A | 1325 | −38.936 | −20.566 | 24.868 | 1.00 | 66.25 | A |
| 2473 | CB | SER | A | 1325 | −38.517 | −19.986 | 23.514 | 1.00 | 65.18 | A |
| 2474 | OG | SER | A | 1325 | −39.679 | −19.461 | 22.827 | 1.00 | 67.74 | A |
| 2475 | C | SER | A | 1325 | −39.968 | −19.748 | 25.699 | 1.00 | 64.07 | A |
| 2476 | O | SER | A | 1325 | −40.500 | −18.778 | 25.201 | 1.00 | 64.07 | A |
| 2477 | N | THR | A | 1326 | −40.144 | −20.141 | 26.967 | 1.00 | 61.78 | A |
| 2478 | CA | THR | A | 1326 | −41.276 | −19.846 | 27.743 | 1.00 | 61.85 | A |
| 2479 | CB | THR | A | 1326 | −41.892 | −21.151 | 28.001 | 1.00 | 63.57 | A |
| 2480 | OG1 | THR | A | 1326 | −43.260 | −20.971 | 28.500 | 1.00 | 63.52 | A |
| 2481 | CG2 | THR | A | 1326 | −40.955 | −21.938 | 28.976 | 1.00 | 64.05 | A |
| 2482 | C | THR | A | 1326 | −41.272 | −19.034 | 29.124 | 1.00 | 62.45 | A |
| 2483 | O | THR | A | 1326 | −42.059 | −18.148 | 29.246 | 1.00 | 61.71 | A |
| 2484 | N | ALA | A | 1327 | −40.492 | −19.383 | 30.153 | 1.00 | 62.02 | A |
| 2485 | CA | ALA | A | 1327 | −40.503 | −18.749 | 31.512 | 1.00 | 60.65 | A |
| 2486 | CB | ALA | A | 1327 | −39.168 | −19.217 | 32.321 | 1.00 | 59.87 | A |
| 2487 | C | ALA | A | 1327 | −40.554 | −17.248 | 31.596 | 1.00 | 60.60 | A |
| 2488 | O | ALA | A | 1327 | −39.537 | −16.580 | 31.826 | 1.00 | 60.44 | A |
| 2489 | N | SER | A | 1328 | −41.712 | −16.654 | 31.512 | 1.00 | 61.78 | A |
| 2490 | CA | SER | A | 1328 | −41.687 | −15.209 | 31.745 | 1.00 | 62.33 | A |
| 2491 | CB | SER | A | 1328 | −43.013 | −14.619 | 32.084 | 1.00 | 61.54 | A |
| 2492 | OG | SER | A | 1328 | −43.915 | −14.902 | 31.091 | 1.00 | 60.82 | A |
| 2493 | C | SER | A | 1328 | −40.813 | −14.936 | 32.925 | 1.00 | 63.76 | A |
| 2494 | O | SER | A | 1328 | −39.820 | −14.263 | 32.767 | 1.00 | 65.62 | A |
| 2495 | N | SER | A | 1329 | −41.216 | −15.380 | 34.116 | 1.00 | 64.82 | A |
| 2496 | CA | SER | A | 1329 | −40.383 | −15.124 | 35.281 | 1.00 | 64.99 | A |
| 2497 | CB | SER | A | 1329 | −41.109 | −15.198 | 36.648 | 1.00 | 65.44 | A |
| 2498 | OG | SER | A | 1329 | −42.504 | −15.192 | 36.583 | 1.00 | 58.86 | A |
| 2499 | C | SER | A | 1329 | −39.453 | −16.253 | 35.221 | 1.00 | 64.80 | A |
| 2500 | O | SER | A | 1329 | −40.007 | −17.379 | 35.073 | 1.00 | 65.86 | A |
| 2501 | N | LYS | A | 1330 | −38.131 | −15.959 | 35.434 | 1.00 | 63.54 | A |
| 2502 | CA | LYS | A | 1330 | −36.974 | −16.882 | 35.418 | 1.00 | 61.70 | A |
| 2503 | CB | LYS | A | 1330 | −35.724 | −16.119 | 35.845 | 1.00 | 62.33 | A |
| 2504 | CG | LYS | A | 1330 | −34.997 | −15.324 | 34.659 | 1.00 | 62.11 | A |
| 2505 | CD | LYS | A | 1330 | −33.524 | −15.324 | 34.795 | 1.00 | 56.81 | A |
| 2506 | CE | LYS | A | 1330 | −32.842 | −14.044 | 34.333 | 1.00 | 61.29 | A |
| 2507 | NZ | LYS | A | 1330 | −33.516 | −12.612 | 34.511 | 1.00 | 60.55 | A |
| 2508 | C | LYS | A | 1330 | −37.107 | −18.188 | 36.232 | 1.00 | 63.44 | A |
| 2509 | O | LYS | A | 1330 | −38.236 | −18.646 | 36.542 | 1.00 | 63.66 | A |
| 2510 | N | ASN | A | 1331 | −35.981 | −18.870 | 36.530 | 1.00 | 65.06 | A |
| 2511 | CA | ASN | A | 1331 | −35.995 | −20.238 | 37.095 | 1.00 | 64.42 | A |
| 2512 | CB | ASN | A | 1331 | −37.112 | −21.093 | 36.514 | 1.00 | 62.48 | A |
| 2513 | CG | ASN | A | 1331 | −36.900 | −22.629 | 36.810 | 1.00 | 67.38 | A |
| 2514 | OD1 | ASN | A | 1331 | −36.078 | −23.312 | 36.125 | 1.00 | 69.59 | A |
| 2515 | ND2 | ASN | A | 1331 | −37.634 | −23.191 | 37.840 | 1.00 | 61.93 | A |
| 2516 | C | ASN | A | 1331 | −34.679 | −20.964 | 36.834 | 1.00 | 66.70 | A |
| 2517 | O | ASN | A | 1331 | −33.678 | −20.409 | 36.251 | 1.00 | 68.54 | A |
| 2518 | N | ALA | A | 1332 | −34.674 | −22.230 | 37.276 | 1.00 | 65.78 | A |
| 2519 | CA | ALA | A | 1332 | −33.560 | −23.109 | 37.111 | 1.00 | 63.19 | A |
| 2520 | CB | ALA | A | 1332 | −33.564 | −24.102 | 38.206 | 1.00 | 64.04 | A |
| 2521 | C | ALA | A | 1332 | −33.596 | −23.845 | 35.807 | 1.00 | 62.23 | A |
| 2522 | O | ALA | A | 1332 | −32.497 | −24.196 | 35.242 | 1.00 | 64.30 | A |
| 2523 | N | SER | A | 1333 | −34.782 | −24.222 | 35.340 | 1.00 | 58.06 | A |
| 2524 | CA | SER | A | 1333 | −34.744 | −24.974 | 34.121 | 1.00 | 55.86 | A |
| 2525 | CB | SER | A | 1333 | −36.068 | −25.504 | 33.621 | 1.00 | 53.81 | A |
| 2526 | OG | SER | A | 1333 | −37.040 | −24.543 | 33.616 | 1.00 | 49.75 | A |
| 2527 | C | SER | A | 1333 | −34.068 | −24.192 | 33.019 | 1.00 | 57.62 | A |
| 2528 | O | SER | A | 1333 | −34.127 | −24.696 | 31.931 | 1.00 | 58.13 | A |
| 2529 | N | CYS | A | 1334 | −33.364 | −23.069 | 33.339 | 1.00 | 57.87 | A |
| 2530 | CA | CYS | A | 1334 | −32.853 | −21.993 | 32.449 | 1.00 | 58.83 | A |
| 2531 | CB | CYS | A | 1334 | −33.441 | −20.670 | 32.890 | 1.00 | 58.51 | A |
| 2532 | SG | CYS | A | 1334 | −35.339 | −20.845 | 32.764 | 1.00 | 69.57 | A |
| 2533 | C | CYS | A | 1334 | −31.359 | −21.869 | 32.291 | 1.00 | 57.97 | A |
| 2534 | O | CYS | A | 1334 | −30.818 | −22.054 | 31.303 | 1.00 | 57.85 | A |
| 2535 | N | TYR | A | 1335 | −30.654 | −21.611 | 33.335 | 1.00 | 60.30 | A |
| 2536 | CA | TYR | A | 1335 | −29.215 | −21.777 | 33.329 | 1.00 | 58.08 | A |
| 2537 | CB | TYR | A | 1335 | −28.795 | −21.870 | 34.760 | 1.00 | 59.91 | A |
| 2538 | CG | TYR | A | 1335 | −29.333 | −20.747 | 35.538 | 1.00 | 60.55 | A |
| 2539 | CD1 | TYR | A | 1335 | −30.003 | −20.964 | 36.679 | 1.00 | 64.79 | A |
| 2540 | CE1 | TYR | A | 1335 | −30.500 | −19.910 | 37.399 | 1.00 | 66.96 | A |
| 2541 | CD2 | TYR | A | 1335 | −29.217 | −19.442 | 35.086 | 1.00 | 63.66 | A |
| 2542 | CE2 | TYR | A | 1335 | −29.675 | −18.345 | 35.821 | 1.00 | 64.41 | A |
| 2543 | CZ | TYR | A | 1335 | −30.312 | −18.578 | 36.991 | 1.00 | 66.87 | A |
| 2544 | OH | TYR | A | 1335 | −30.840 | −17.474 | 37.730 | 1.00 | 70.42 | A |
| 2545 | C | TYR | A | 1335 | −28.611 | −22.900 | 32.491 | 1.00 | 58.29 | A |
| 2546 | O | TYR | A | 1335 | −29.307 | −23.794 | 32.067 | 1.00 | 60.26 | A |
| 2547 | N | PHE | A | 1336 | −27.301 | −22.781 | 32.177 | 1.00 | 56.15 | A |
| 2548 | CA | PHE | A | 1336 | −26.532 | −23.643 | 31.179 | 1.00 | 50.42 | A |
| 2549 | CB | PHE | A | 1336 | −26.869 | −23.331 | 29.720 | 1.00 | 45.55 | A |
| 2550 | CG | PHE | A | 1336 | −28.232 | −23.509 | 29.380 | 1.00 | 40.17 | A |
| 2551 | CD1 | PHE | A | 1336 | −28.654 | −24.634 | 28.881 | 1.00 | 40.89 | A |
| 2552 | CD2 | PHE | A | 1336 | −29.136 | −22.492 | 29.428 | 1.00 | 41.13 | A |
| 2553 | CE1 | PHE | A | 1336 | −29.996 | −24.783 | 28.483 | 1.00 | 33.95 | A |
| 2554 | CE2 | PHE | A | 1336 | −30.492 | −22.708 | 29.021 | 1.00 | 26.67 | A |
| 2555 | CZ | PHE | A | 1336 | −30.874 | −23.877 | 28.693 | 1.00 | 31.66 | A |
| 2556 | C | PHE | A | 1336 | −25.146 | −23.156 | 31.385 | 1.00 | 47.56 | A |
| 2557 | O | PHE | A | 1336 | −24.989 | −21.985 | 31.748 | 1.00 | 47.15 | A |
| 2558 | N | ASP | A | 1337 | −24.214 | −24.054 | 31.173 | 1.00 | 45.85 | A |
| 2559 | CA | ASP | A | 1337 | −22.814 | −23.845 | 31.240 | 1.00 | 46.34 | A |
| 2560 | CB | ASP | A | 1337 | −22.139 | −24.819 | 32.169 | 1.00 | 45.98 | A |
| 2561 | CG | ASP | A | 1337 | −23.013 | −25.026 | 33.535 | 1.00 | 51.61 | A |
| 2562 | OD1 | ASP | A | 1337 | −23.100 | −26.211 | 34.055 | 1.00 | 44.12 | A |
| 2563 | OD2 | ASP | A | 1337 | −23.629 | −23.982 | 33.998 | 1.00 | 45.14 | A |
| 2564 | C | ASP | A | 1337 | −22.427 | −24.163 | 29.901 | 1.00 | 48.11 | A |
| 2565 | O | ASP | A | 1337 | −22.780 | −25.186 | 29.282 | 1.00 | 49.94 | A |
| 2566 | N | ILE | A | 1338 | −21.631 | −23.243 | 29.470 | 1.00 | 48.80 | A |
| 2567 | CA | ILE | A | 1338 | −21.127 | −23.197 | 28.261 | 1.00 | 48.93 | A |
| 2568 | CB | ILE | A | 1338 | −20.900 | −21.719 | 27.990 | 1.00 | 49.42 | A |
| 2569 | CG2 | ILE | A | 1338 | −19.987 | −21.441 | 26.783 | 1.00 | 54.39 | A |
| 2570 | CG1 | ILE | A | 1338 | −22.158 | −21.024 | 27.591 | 1.00 | 48.37 | A |
| 2571 | CD1 | ILE | A | 1338 | −22.938 | −20.618 | 28.704 | 1.00 | 44.23 | A |
| 2572 | C | ILE | A | 1338 | −19.835 | −23.761 | 28.604 | 1.00 | 50.17 | A |
| 2573 | O | ILE | A | 1338 | −19.231 | −23.351 | 29.561 | 1.00 | 51.43 | A |
| 2574 | N | GLU | A | 1339 | −19.378 | −24.635 | 27.727 | 1.00 | 53.83 | A |
| 2575 | CA | GLU | A | 1339 | −17.959 | −25.079 | 27.469 | 1.00 | 54.30 | A |
| 2576 | CB | GLU | A | 1339 | −18.127 | −26.628 | 27.264 | 1.00 | 54.36 | A |
| 2577 | CG | GLU | A | 1339 | −16.893 | −27.495 | 26.975 | 1.00 | 54.17 | A |
| 2578 | CD | GLU | A | 1339 | −17.355 | −28.858 | 26.803 | 1.00 | 56.08 | A |
| 2579 | OE1 | GLU | A | 1339 | −18.611 | −28.926 | 26.908 | 1.00 | 58.76 | A |
| 2580 | OE2 | GLU | A | 1339 | −16.524 | −29.817 | 26.655 | 1.00 | 54.05 | A |
| 2581 | C | GLU | A | 1339 | −17.254 | −24.398 | 26.213 | 1.00 | 54.49 | A |
| 2582 | O | GLU | A | 1339 | −17.472 | −24.759 | 25.042 | 1.00 | 56.52 | A |
| 2583 | N | TRP | A | 1340 | −16.405 | −23.412 | 26.440 | 1.00 | 55.66 | A |
| 2584 | CA | TRP | A | 1340 | −15.752 | −22.490 | 25.417 | 1.00 | 53.15 | A |
| 2585 | CB | TRP | A | 1340 | −15.096 | −21.167 | 26.093 | 1.00 | 51.55 | A |
| 2586 | CG | TRP | A | 1340 | −16.134 | −20.350 | 26.970 | 1.00 | 51.60 | A |
| 2587 | CD2 | TRP | A | 1340 | −17.123 | −19.321 | 26.468 | 1.00 | 47.83 | A |
| 2588 | CE2 | TRP | A | 1340 | −17.903 | −18.905 | 27.590 | 1.00 | 49.29 | A |
| 2589 | CE3 | TRP | A | 1340 | −17.397 | −18.778 | 25.209 | 1.00 | 37.38 | A |
| 2590 | CD1 | TRP | A | 1340 | −16.326 | −20.457 | 28.285 | 1.00 | 44.12 | A |
| 2591 | NE1 | TRP | A | 1340 | −17.403 | −19.575 | 28.680 | 1.00 | 44.14 | A |
| 2592 | CZ2 | TRP | A | 1340 | −19.009 | −17.958 | 27.463 | 1.00 | 55.28 | A |
| 2593 | CZ3 | TRP | A | 1340 | −18.569 | −17.936 | 25.010 | 1.00 | 42.69 | A |
| 2594 | CH2 | TRP | A | 1340 | −19.309 | −17.470 | 26.124 | 1.00 | 51.78 | A |
| 2595 | C | TRP | A | 1340 | −14.743 | −23.214 | 24.612 | 1.00 | 52.81 | A |
| 2596 | O | TRP | A | 1340 | −13.522 | −22.963 | 24.635 | 1.00 | 54.03 | A |
| 2597 | N | ARG | A | 1341 | −15.193 | −24.092 | 23.775 | 1.00 | 55.65 | A |
| 2598 | CA | ARG | A | 1341 | −14.187 | −24.763 | 22.893 | 1.00 | 55.98 | A |
| 2599 | CB | ARG | A | 1341 | −14.724 | −26.039 | 22.335 | 1.00 | 57.64 | A |
| 2600 | CG | ARG | A | 1341 | −15.034 | −27.122 | 23.335 | 1.00 | 59.51 | A |
| 2601 | CD | ARG | A | 1341 | −15.800 | −28.121 | 22.452 | 1.00 | 64.66 | A |
| 2602 | NE | ARG | A | 1341 | −15.124 | −29.321 | 21.929 | 1.00 | 68.75 | A |
| 2603 | CZ | ARG | A | 1341 | −14.712 | −29.511 | 20.656 | 1.00 | 74.56 | A |
| 2604 | NH1 | ARG | A | 1341 | −14.793 | −28.485 | 19.770 | 1.00 | 68.76 | A |
| 2605 | NH2 | ARG | A | 1341 | −14.245 | −30.759 | 20.237 | 1.00 | 70.14 | A |
| 2606 | C | ARG | A | 1341 | −13.690 | −23.910 | 21.775 | 1.00 | 54.75 | A |
| 2607 | O | ARG | A | 1341 | −13.320 | −24.386 | 20.680 | 1.00 | 57.08 | A |
| 2608 | N | ASP | A | 1342 | −13.612 | −22.646 | 22.134 | 1.00 | 51.90 | A |
| 2609 | CA | ASP | A | 1342 | −13.345 | −21.443 | 21.273 | 1.00 | 50.64 | A |
| 2610 | CB | ASP | A | 1342 | −12.028 | −20.699 | 21.773 | 1.00 | 49.44 | A |
| 2611 | CG | ASP | A | 1342 | −10.859 | −21.681 | 21.825 | 1.00 | 53.74 | A |
| 2612 | OD1 | ASP | A | 1342 | −11.088 | −22.692 | 22.478 | 1.00 | 63.21 | A |
| 2613 | OD2 | ASP | A | 1342 | −9.787 | −21.552 | 21.194 | 1.00 | 56.52 | A |
| 2614 | C | ASP | A | 1342 | −13.382 | −21.621 | 19.702 | 1.00 | 48.40 | A |
| 2615 | O | ASP | A | 1342 | −12.377 | −21.924 | 19.132 | 1.00 | 44.85 | A |
| 2616 | N | ARG | A | 1343 | −14.538 | −21.343 | 19.076 | 1.00 | 46.46 | A |
| 2617 | CA | ARG | A | 1343 | −14.865 | −21.449 | 17.565 | 1.00 | 45.77 | A |
| 2618 | CB | ARG | A | 1343 | −13.922 | −22.329 | 16.781 | 1.00 | 43.51 | A |
| 2619 | CG | ARG | A | 1343 | −12.804 | −21.516 | 16.185 | 1.00 | 44.37 | A |
| 2620 | CD | ARG | A | 1343 | −12.888 | −21.327 | 14.557 | 1.00 | 45.61 | A |
| 2621 | NE | ARG | A | 1343 | −12.121 | −20.221 | 14.054 | 1.00 | 34.77 | A |
| 2622 | CZ | ARG | A | 1343 | −10.765 | −20.171 | 14.030 | 1.00 | 40.11 | A |
| 2623 | NH1 | ARG | A | 1343 | −9.982 | −21.188 | 14.509 | 1.00 | 32.49 | A |
| 2624 | NH2 | ARG | A | 1343 | −10.137 | −19.091 | 13.453 | 1.00 | 35.12 | A |
| 2625 | C | ARG | A | 1343 | −16.301 | −21.943 | 17.478 | 1.00 | 46.67 | A |
| 2626 | O | ARG | A | 1343 | −17.148 | −21.322 | 16.880 | 1.00 | 47.71 | A |
| 2627 | N | ARG | A | 1344 | −16.599 | −22.994 | 18.243 | 1.00 | 46.99 | A |
| 2628 | CA | ARG | A | 1344 | −17.942 | −23.430 | 18.509 | 1.00 | 46.73 | A |
| 2629 | CB | ARG | A | 1344 | −18.097 | −24.803 | 18.005 | 1.00 | 47.01 | A |
| 2630 | CG | ARG | A | 1344 | −18.240 | −24.689 | 16.531 | 1.00 | 53.81 | A |
| 2631 | CD | ARG | A | 1344 | −16.895 | −24.852 | 15.739 | 1.00 | 64.20 | A |
| 2632 | NE | ARG | A | 1344 | −17.326 | −24.580 | 14.405 | 1.00 | 69.32 | A |
| 2633 | CZ | ARG | A | 1344 | −17.558 | −23.375 | 13.896 | 1.00 | 69.64 | A |
| 2634 | NH1 | ARG | A | 1344 | −18.040 | −23.373 | 12.639 | 1.00 | 66.38 | A |
| 2635 | NH2 | ARG | A | 1344 | −17.277 | −22.244 | 14.579 | 1.00 | 57.92 | A |
| 2636 | C | ARG | A | 1344 | −18.007 | −23.517 | 19.983 | 1.00 | 46.09 | A |
| 2637 | O | ARG | A | 1344 | −17.009 | −23.392 | 20.589 | 1.00 | 43.47 | A |
| 2638 | N | ILE | A | 1345 | −19.191 | −23.863 | 20.525 | 1.00 | 49.79 | A |
| 2639 | CA | ILE | A | 1345 | −19.618 | −23.983 | 21.986 | 1.00 | 47.28 | A |
| 2640 | CB | ILE | A | 1345 | −20.314 | −22.621 | 22.275 | 1.00 | 48.22 | A |
| 2641 | CG2 | ILE | A | 1345 | −21.983 | −22.556 | 22.481 | 1.00 | 43.66 | A |
| 2642 | CG1 | ILE | A | 1345 | −19.444 | −21.836 | 23.228 | 1.00 | 44.60 | A |
| 2643 | CD1 | ILE | A | 1345 | −20.345 | −21.238 | 24.256 | 1.00 | 52.66 | A |
| 2644 | C | ILE | A | 1345 | −20.500 | −25.240 | 22.353 | 1.00 | 47.76 | A |
| 2645 | O | ILE | A | 1345 | −20.717 | −26.108 | 21.542 | 1.00 | 47.71 | A |
| 2646 | N | THR | A | 1346 | −20.879 | −25.381 | 23.628 | 1.00 | 48.55 | A |
| 2647 | CA | THR | A | 1346 | −21.665 | −26.478 | 24.142 | 1.00 | 47.58 | A |
| 2648 | CB | THR | A | 1346 | −20.809 | −27.524 | 24.963 | 1.00 | 47.95 | A |
| 2649 | OG1 | THR | A | 1346 | −19.575 | −27.745 | 24.323 | 1.00 | 47.03 | A |
| 2650 | CG2 | THR | A | 1346 | −21.473 | −28.992 | 24.974 | 1.00 | 50.44 | A |
| 2651 | C | THR | A | 1346 | −22.572 | −25.742 | 25.075 | 1.00 | 48.87 | A |
| 2652 | O | THR | A | 1346 | −22.435 | −24.540 | 25.216 | 1.00 | 50.92 | A |
| 2653 | N | LEU | A | 1347 | −23.495 | −26.422 | 25.735 | 1.00 | 48.96 | A |
| 2654 | CA | LEU | A | 1347 | −24.449 | −25.756 | 26.508 | 1.00 | 49.28 | A |
| 2655 | CB | LEU | A | 1347 | −25.624 | −25.274 | 25.608 | 1.00 | 49.21 | A |
| 2656 | CG | LEU | A | 1347 | −26.126 | −23.883 | 26.132 | 1.00 | 47.79 | A |
| 2657 | CD1 | LEU | A | 1347 | −25.091 | −23.051 | 25.671 | 1.00 | 45.88 | A |
| 2658 | CD2 | LEU | A | 1347 | −27.407 | −23.327 | 25.601 | 1.00 | 47.09 | A |
| 2659 | C | LEU | A | 1347 | −24.797 | −26.903 | 27.415 | 1.00 | 51.68 | A |
| 2660 | O | LEU | A | 1347 | −25.294 | −27.936 | 26.920 | 1.00 | 50.08 | A |
| 2661 | N | ARG | A | 1348 | −24.436 | −26.832 | 28.738 | 1.00 | 53.43 | A |
| 2662 | CA | ARG | A | 1348 | −24.661 | −28.088 | 29.563 | 1.00 | 54.18 | A |
| 2663 | CB | ARG | A | 1348 | −23.611 | −28.409 | 30.622 | 1.00 | 53.34 | A |
| 2664 | CG | ARG | A | 1348 | −23.596 | −29.868 | 31.201 | 1.00 | 51.22 | A |
| 2665 | CD | ARG | A | 1348 | −23.016 | −29.879 | 32.651 | 1.00 | 52.22 | A |
| 2666 | NE | ARG | A | 1348 | −21.554 | −29.717 | 32.635 | 1.00 | 52.76 | A |
| 2667 | CZ | ARG | A | 1348 | −20.549 | −30.512 | 33.023 | 1.00 | 50.60 | A |
| 2668 | NH1 | ARG | A | 1348 | −20.782 | −31.682 | 33.589 | 1.00 | 56.73 | A |
| 2669 | NH2 | ARG | A | 1348 | −19.246 | −30.117 | 32.779 | 1.00 | 45.15 | A |
| 2670 | C | ARG | A | 1348 | −25.865 | −27.631 | 30.182 | 1.00 | 55.44 | A |
| 2671 | O | ARG | A | 1348 | −25.821 | −26.545 | 30.727 | 1.00 | 57.37 | A |
| 2672 | N | ALA | A | 1349 | −26.920 | −28.422 | 30.095 | 1.00 | 55.73 | A |
| 2673 | CA | ALA | A | 1349 | −28.238 | −28.058 | 30.614 | 1.00 | 56.45 | A |
| 2674 | CB | ALA | A | 1349 | −29.360 | −28.936 | 29.942 | 1.00 | 56.37 | A |
| 2675 | C | ALA | A | 1349 | −28.379 | −28.257 | 32.062 | 1.00 | 57.22 | A |
| 2676 | O | ALA | A | 1349 | −27.420 | −28.465 | 32.802 | 1.00 | 54.98 | A |
| 2677 | N | SER | A | 1350 | −29.640 | −28.268 | 32.456 | 1.00 | 59.48 | A |
| 2678 | CA | SER | A | 1350 | −29.981 | −28.572 | 33.796 | 1.00 | 61.95 | A |
| 2679 | CB | SER | A | 1350 | −31.211 | −27.717 | 34.099 | 1.00 | 62.70 | A |
| 2680 | OG | SER | A | 1350 | −31.362 | −27.532 | 35.475 | 1.00 | 68.52 | A |
| 2681 | C | SER | A | 1350 | −30.170 | −30.156 | 33.950 | 1.00 | 62.81 | A |
| 2682 | O | SER | A | 1350 | −29.759 | −30.750 | 34.975 | 1.00 | 66.06 | A |
| 2683 | N | ASN | A | 1351 | −30.724 | −30.896 | 32.999 | 1.00 | 59.48 | A |
| 2684 | CA | ASN | A | 1351 | −30.531 | −32.280 | 33.236 | 1.00 | 56.89 | A |
| 2685 | CB | ASN | A | 1351 | −31.495 | −33.109 | 32.416 | 1.00 | 57.32 | A |
| 2686 | CG | ASN | A | 1351 | −31.450 | −32.820 | 30.910 | 1.00 | 52.77 | A |
| 2687 | OD1 | ASN | A | 1351 | −30.675 | −32.079 | 30.390 | 1.00 | 51.55 | A |
| 2688 | ND2 | ASN | A | 1351 | −32.332 | −33.475 | 30.228 | 1.00 | 51.95 | A |
| 2689 | C | ASN | A | 1351 | −29.050 | −32.728 | 33.099 | 1.00 | 58.49 | A |
| 2690 | O | ASN | A | 1351 | −28.738 | −33.903 | 32.832 | 1.00 | 56.41 | A |
| 2691 | N | GLY | A | 1352 | −28.107 | −31.798 | 33.318 | 1.00 | 59.63 | A |
| 2692 | CA | GLY | A | 1352 | −26.665 | −32.067 | 33.111 | 1.00 | 59.95 | A |
| 2693 | C | GLY | A | 1352 | −26.323 | −32.681 | 31.751 | 1.00 | 60.84 | A |
| 2694 | O | GLY | A | 1352 | −25.125 | −32.865 | 31.414 | 1.00 | 60.45 | A |
| 2695 | N | LYS | A | 1353 | −27.356 | −33.061 | 31.005 | 1.00 | 62.37 | A |
| 2696 | CA | LYS | A | 1353 | −27.251 | −33.698 | 29.628 | 1.00 | 64.40 | A |
| 2697 | CB | LYS | A | 1353 | −28.646 | −34.129 | 29.158 | 1.00 | 63.45 | A |
| 2698 | CG | LYS | A | 1353 | −29.334 | −35.076 | 30.128 | 1.00 | 60.26 | A |
| 2699 | CD | LYS | A | 1353 | −29.141 | −36.571 | 29.794 | 1.00 | 51.21 | A |
| 2700 | CE | LYS | A | 1353 | −29.266 | −37.463 | 30.963 | 1.00 | 39.98 | A |
| 2701 | NZ | LYS | A | 1353 | −30.496 | −37.017 | 31.969 | 1.00 | 39.61 | A |
| 2702 | C | LYS | A | 1353 | −26.747 | −32.697 | 28.585 | 1.00 | 65.41 | A |
| 2703 | O | LYS | A | 1353 | −26.696 | −31.492 | 28.937 | 1.00 | 68.97 | A |
| 2704 | N | PHE | A | 1354 | −26.452 | −33.124 | 27.327 | 1.00 | 64.50 | A |
| 2705 | CA | PHE | A | 1354 | −26.225 | −32.125 | 26.192 | 1.00 | 63.23 | A |
| 2706 | CB | PHE | A | 1354 | −24.838 | −32.279 | 25.546 | 1.00 | 63.91 | A |
| 2707 | CG | PHE | A | 1354 | −23.716 | −32.048 | 26.566 | 1.00 | 70.21 | A |
| 2708 | CD1 | PHE | A | 1354 | −23.673 | −30.864 | 27.322 | 1.00 | 73.78 | A |
| 2709 | CD2 | PHE | A | 1354 | −22.748 | −33.043 | 26.846 | 1.00 | 72.11 | A |
| 2710 | CE1 | PHE | A | 1354 | −22.647 | −30.705 | 28.261 | 1.00 | 77.23 | A |
| 2711 | CE2 | PHE | A | 1354 | −21.732 | −32.886 | 27.788 | 1.00 | 66.14 | A |
| 2712 | CZ | PHE | A | 1354 | −21.686 | −31.767 | 28.492 | 1.00 | 71.64 | A |
| 2713 | C | PHE | A | 1354 | −27.340 | −31.367 | 25.325 | 1.00 | 60.44 | A |
| 2714 | O | PHE | A | 1354 | −28.352 | −31.891 | 24.976 | 1.00 | 59.27 | A |
| 2715 | N | VAL | A | 1355 | −27.208 | −30.095 | 25.036 | 1.00 | 58.88 | A |
| 2716 | CA | VAL | A | 1355 | −28.205 | −29.609 | 24.144 | 1.00 | 58.29 | A |
| 2717 | CB | VAL | A | 1355 | −28.497 | −28.172 | 24.305 | 1.00 | 60.19 | A |
| 2718 | CG1 | VAL | A | 1355 | −29.412 | −27.757 | 23.171 | 1.00 | 57.84 | A |
| 2719 | CG2 | VAL | A | 1355 | −29.090 | −27.881 | 25.788 | 1.00 | 56.65 | A |
| 2720 | C | VAL | A | 1355 | −27.905 | −30.015 | 22.699 | 1.00 | 58.93 | A |
| 2721 | O | VAL | A | 1355 | −26.790 | −29.787 | 22.170 | 1.00 | 58.73 | A |
| 2722 | N | THR | A | 1356 | −28.844 | −30.817 | 22.147 | 1.00 | 59.71 | A |
| 2723 | CA | THR | A | 1356 | −28.761 | −31.250 | 20.716 | 1.00 | 58.29 | A |
| 2724 | CB | THR | A | 1356 | −28.459 | −32.836 | 20.459 | 1.00 | 56.92 | A |
| 2725 | OG1 | THR | A | 1356 | −27.265 | −32.934 | 19.586 | 1.00 | 54.96 | A |
| 2726 | CG2 | THR | A | 1356 | −29.573 | −33.473 | 19.701 | 1.00 | 54.19 | A |
| 2727 | C | THR | A | 1356 | −29.841 | −30.581 | 19.778 | 1.00 | 56.27 | A |
| 2728 | O | THR | A | 1356 | −30.865 | −30.172 | 20.272 | 1.00 | 52.39 | A |
| 2729 | N | SER | A | 1357 | −29.546 | −30.550 | 18.472 | 1.00 | 56.37 | A |
| 2730 | CA | SER | A | 1357 | −30.489 | −30.393 | 17.329 | 1.00 | 59.51 | A |
| 2731 | CB | SER | A | 1357 | −29.626 | −29.735 | 16.188 | 1.00 | 59.21 | A |
| 2732 | OG | SER | A | 1357 | −28.722 | −30.763 | 15.671 | 1.00 | 57.32 | A |
| 2733 | C | SER | A | 1357 | −31.255 | −31.705 | 16.714 | 1.00 | 59.54 | A |
| 2734 | O | SER | A | 1357 | −30.875 | −32.247 | 15.669 | 1.00 | 59.56 | A |
| 2735 | N | LYS | A | 1358 | −32.310 | −32.217 | 17.301 | 1.00 | 59.83 | A |
| 2736 | CA | LYS | A | 1358 | −33.005 | −33.400 | 16.684 | 1.00 | 62.57 | A |
| 2737 | CB | LYS | A | 1358 | −34.149 | −33.964 | 17.596 | 1.00 | 62.50 | A |
| 2738 | CG | LYS | A | 1358 | −33.810 | −34.409 | 19.102 | 1.00 | 63.70 | A |
| 2739 | CD | LYS | A | 1358 | −34.898 | −35.317 | 19.945 | 1.00 | 63.05 | A |
| 2740 | CE | LYS | A | 1358 | −36.414 | −34.984 | 19.741 | 1.00 | 58.01 | A |
| 2741 | NZ | LYS | A | 1358 | −37.309 | −36.176 | 20.144 | 1.00 | 55.33 | A |
| 2742 | C | LYS | A | 1358 | −33.590 | −33.221 | 15.206 | 1.00 | 63.76 | A |
| 2743 | O | LYS | A | 1358 | −33.949 | −32.133 | 14.716 | 1.00 | 61.20 | A |
| 2744 | N | LYS | A | 1359 | −33.650 | −34.343 | 14.466 | 1.00 | 67.46 | A |
| 2745 | CA | LYS | A | 1359 | −34.259 | −34.308 | 13.103 | 1.00 | 67.31 | A |
| 2746 | CB | LYS | A | 1359 | −34.564 | −35.769 | 12.622 | 1.00 | 68.61 | A |
| 2747 | CG | LYS | A | 1359 | −33.233 | −36.733 | 12.440 | 1.00 | 69.22 | A |
| 2748 | CD | LYS | A | 1359 | −33.375 | −38.247 | 12.876 | 1.00 | 66.64 | A |
| 2749 | CE | LYS | A | 1359 | −34.821 | −38.894 | 12.782 | 1.00 | 66.66 | A |
| 2750 | NZ | LYS | A | 1359 | −35.863 | −38.766 | 13.976 | 1.00 | 56.99 | A |
| 2751 | C | LYS | A | 1359 | −35.501 | −33.267 | 13.134 | 1.00 | 68.64 | A |
| 2752 | O | LYS | A | 1359 | −35.708 | −32.435 | 12.233 | 1.00 | 67.27 | A |
| 2753 | N | ASN | A | 1360 | −36.259 | −33.230 | 14.232 | 1.00 | 68.84 | A |
| 2754 | CA | ASN | A | 1360 | −37.390 | −32.281 | 14.236 | 1.00 | 69.53 | A |
| 2755 | CB | ASN | A | 1360 | −38.417 | −32.484 | 15.392 | 1.00 | 69.24 | A |
| 2756 | CG | ASN | A | 1360 | −37.807 | −32.919 | 16.715 | 1.00 | 67.27 | A |
| 2757 | OD1 | ASN | A | 1360 | −37.317 | −32.125 | 17.443 | 1.00 | 69.33 | A |
| 2758 | ND2 | ASN | A | 1360 | −37.971 | −34.187 | 17.075 | 1.00 | 74.51 | A |
| 2759 | C | ASN | A | 1360 | −37.002 | −30.782 | 14.131 | 1.00 | 68.76 | A |
| 2760 | O | ASN | A | 1360 | −37.862 | −29.904 | 14.225 | 1.00 | 68.66 | A |
| 2761 | N | GLY | A | 1361 | −35.707 | −30.509 | 14.017 | 1.00 | 67.01 | A |
| 2762 | CA | GLY | A | 1361 | −35.250 | −29.172 | 14.277 | 1.00 | 63.70 | A |
| 2763 | C | GLY | A | 1361 | −34.876 | −28.919 | 15.722 | 1.00 | 61.67 | A |
| 2764 | O | GLY | A | 1361 | −34.062 | −28.074 | 15.963 | 1.00 | 61.39 | A |
| 2765 | N | GLN | A | 1362 | −35.451 | −29.619 | 16.703 | 1.00 | 60.09 | A |
| 2766 | CA | GLN | A | 1362 | −35.673 | −28.934 | 18.028 | 1.00 | 60.12 | A |
| 2767 | CB | GLN | A | 1362 | −37.060 | −29.313 | 18.746 | 1.00 | 59.64 | A |
| 2768 | CG | GLN | A | 1362 | −36.754 | −30.424 | 19.995 | 1.00 | 60.06 | A |
| 2769 | CD | GLN | A | 1362 | −37.686 | −30.419 | 21.160 | 1.00 | 56.60 | A |
| 2770 | OE1 | GLN | A | 1362 | −37.811 | −29.426 | 21.896 | 1.00 | 60.76 | A |
| 2771 | NE2 | GLN | A | 1362 | −38.385 | −31.525 | 21.336 | 1.00 | 53.51 | A |
| 2772 | C | GLN | A | 1362 | −34.459 | −29.086 | 18.965 | 1.00 | 59.18 | A |
| 2773 | O | GLN | A | 1362 | −33.622 | −29.857 | 18.631 | 1.00 | 58.74 | A |
| 2774 | N | LEU | A | 1363 | −34.449 | −28.441 | 20.138 | 1.00 | 59.07 | A |
| 2775 | CA | LEU | A | 1363 | −33.349 | −28.515 | 21.042 | 1.00 | 60.64 | A |
| 2776 | CB | LEU | A | 1363 | −32.976 | −27.160 | 21.652 | 1.00 | 60.07 | A |
| 2777 | CG | LEU | A | 1363 | −32.746 | −25.861 | 20.821 | 1.00 | 58.30 | A |
| 2778 | CD1 | LEU | A | 1363 | −33.011 | −24.635 | 21.760 | 1.00 | 52.04 | A |
| 2779 | CD2 | LEU | A | 1363 | −31.469 | −25.732 | 19.939 | 1.00 | 45.48 | A |
| 2780 | C | LEU | A | 1363 | −33.571 | −29.488 | 22.180 | 1.00 | 63.17 | A |
| 2781 | O | LEU | A | 1363 | −34.393 | −29.304 | 23.087 | 1.00 | 64.95 | A |
| 2782 | N | ALA | A | 1364 | −32.739 | −30.522 | 22.148 | 1.00 | 64.37 | A |
| 2783 | CA | ALA | A | 1364 | −32.851 | −31.689 | 22.968 | 1.00 | 61.98 | A |
| 2784 | CB | ALA | A | 1364 | −33.086 | −32.814 | 22.071 | 1.00 | 62.73 | A |
| 2785 | C | ALA | A | 1364 | −31.577 | −31.812 | 23.782 | 1.00 | 61.15 | A |
| 2786 | O | ALA | A | 1364 | −30.441 | −32.054 | 23.313 | 1.00 | 57.82 | A |
| 2787 | N | ALA | A | 1365 | −31.810 | −31.390 | 25.006 | 1.00 | 62.12 | A |
| 2788 | CA | ALA | A | 1365 | −30.918 | −31.595 | 26.183 | 1.00 | 61.05 | A |
| 2789 | CB | ALA | A | 1365 | −31.395 | −30.716 | 27.258 | 1.00 | 59.93 | A |
| 2790 | C | ALA | A | 1365 | −30.908 | −33.084 | 26.665 | 1.00 | 59.92 | A |
| 2791 | O | ALA | A | 1365 | −31.401 | −33.449 | 27.744 | 1.00 | 59.86 | A |
| 2792 | N | SER | A | 1366 | −30.359 | −33.946 | 25.827 | 1.00 | 59.25 | A |
| 2793 | CA | SER | A | 1366 | −30.474 | −35.407 | 25.970 | 1.00 | 57.85 | A |
| 2794 | CB | SER | A | 1366 | −31.135 | −35.963 | 24.744 | 1.00 | 57.21 | A |
| 2795 | OG | SER | A | 1366 | −32.436 | −35.427 | 24.683 | 1.00 | 60.14 | A |
| 2796 | C | SER | A | 1366 | −29.120 | −35.966 | 26.052 | 1.00 | 56.90 | A |
| 2797 | O | SER | A | 1366 | −28.773 | −36.563 | 27.082 | 1.00 | 52.77 | A |
| 2798 | N | VAL | A | 1367 | −28.327 | −35.706 | 25.001 | 1.00 | 57.02 | A |
| 2799 | CA | VAL | A | 1367 | −26.893 | −36.151 | 25.015 | 1.00 | 59.38 | A |
| 2800 | CB | VAL | A | 1367 | −25.997 | −35.435 | 23.978 | 1.00 | 59.06 | A |
| 2801 | CG1 | VAL | A | 1367 | −24.973 | −36.466 | 23.402 | 1.00 | 58.89 | A |
| 2802 | CG2 | VAL | A | 1367 | −26.805 | −34.879 | 22.804 | 1.00 | 58.45 | A |
| 2803 | C | VAL | A | 1367 | −26.248 | −36.203 | 26.402 | 1.00 | 61.66 | A |
| 2804 | O | VAL | A | 1367 | −26.726 | −35.519 | 27.350 | 1.00 | 64.82 | A |
| 2805 | N | GLU | A | 1368 | −25.229 | −37.036 | 26.618 | 1.00 | 62.30 | A |
| 2806 | CA | GLU | A | 1368 | −24.514 | −36.881 | 27.951 | 1.00 | 62.88 | A |
| 2807 | CB | GLU | A | 1368 | −24.635 | −38.090 | 29.023 | 1.00 | 60.14 | A |
| 2808 | CG | GLU | A | 1368 | −26.064 | −38.506 | 29.278 | 1.00 | 59.78 | A |
| 2809 | CD | GLU | A | 1368 | −26.312 | −39.549 | 30.359 | 1.00 | 62.94 | A |
| 2810 | OE1 | GLU | A | 1368 | −27.386 | −40.218 | 30.341 | 1.00 | 55.63 | A |
| 2811 | OE2 | GLU | A | 1368 | −25.457 | −39.668 | 31.268 | 1.00 | 70.93 | A |
| 2812 | C | GLU | A | 1368 | −23.066 | −36.543 | 27.637 | 1.00 | 63.23 | A |
| 2813 | O | GLU | A | 1368 | −22.360 | −36.257 | 28.605 | 1.00 | 62.99 | A |
| 2814 | N | THR | A | 1369 | −22.624 | −36.662 | 26.346 | 1.00 | 62.54 | A |
| 2815 | CA | THR | A | 1369 | −21.233 | −36.335 | 25.969 | 1.00 | 62.23 | A |
| 2816 | CB | THR | A | 1369 | −20.442 | −37.490 | 25.497 | 1.00 | 63.31 | A |
| 2817 | OG1 | THR | A | 1369 | −21.012 | −38.708 | 26.030 | 1.00 | 68.84 | A |
| 2818 | CG2 | THR | A | 1369 | −18.967 | −37.302 | 25.912 | 1.00 | 56.11 | A |
| 2819 | C | THR | A | 1369 | −21.217 | −35.363 | 24.870 | 1.00 | 63.01 | A |
| 2820 | O | THR | A | 1369 | −22.183 | −35.321 | 24.068 | 1.00 | 64.84 | A |
| 2821 | N | ALA | A | 1370 | −20.164 | −34.549 | 24.830 | 1.00 | 61.96 | A |
| 2822 | CA | ALA | A | 1370 | −20.172 | −33.416 | 23.895 | 1.00 | 62.59 | A |
| 2823 | CB | ALA | A | 1370 | −19.118 | −32.342 | 24.336 | 1.00 | 63.83 | A |
| 2824 | C | ALA | A | 1370 | −19.871 | −33.976 | 22.501 | 1.00 | 62.10 | A |
| 2825 | O | ALA | A | 1370 | −18.715 | −34.345 | 22.227 | 1.00 | 63.18 | A |
| 2826 | N | GLY | A | 1371 | −20.892 | −34.108 | 21.648 | 1.00 | 60.40 | A |
| 2827 | CA | GLY | A | 1371 | −20.773 | −34.970 | 20.506 | 1.00 | 58.60 | A |
| 2828 | C | GLY | A | 1371 | −19.939 | −34.411 | 19.456 | 1.00 | 58.08 | A |
| 2829 | O | GLY | A | 1371 | −18.817 | −34.773 | 19.289 | 1.00 | 59.80 | A |
| 2830 | N | ASP | A | 1372 | −20.491 | −33.414 | 18.807 | 1.00 | 59.75 | A |
| 2831 | CA | ASP | A | 1372 | −20.010 | −32.835 | 17.464 | 1.00 | 60.25 | A |
| 2832 | CB | ASP | A | 1372 | −18.870 | −33.568 | 16.645 | 1.00 | 58.55 | A |
| 2833 | CG | ASP | A | 1372 | −18.037 | −32.542 | 15.950 | 1.00 | 59.09 | A |
| 2834 | OD1 | ASP | A | 1372 | −16.851 | −32.774 | 15.522 | 1.00 | 61.64 | A |
| 2835 | OD2 | ASP | A | 1372 | −18.619 | −31.420 | 15.951 | 1.00 | 49.16 | A |
| 2836 | C | ASP | A | 1372 | −21.195 | −32.364 | 16.542 | 1.00 | 57.63 | A |
| 2837 | O | ASP | A | 1372 | −21.160 | −31.270 | 15.924 | 1.00 | 58.29 | A |
| 2838 | N | SER | A | 1373 | −22.237 | −33.134 | 16.607 | 1.00 | 54.78 | A |
| 2839 | CA | SER | A | 1373 | −23.547 | −32.668 | 16.301 | 1.00 | 58.17 | A |
| 2840 | CB | SER | A | 1373 | −24.329 | −33.900 | 15.662 | 1.00 | 58.40 | A |
| 2841 | OG | SER | A | 1373 | −23.386 | −34.754 | 14.832 | 1.00 | 55.35 | A |
| 2842 | C | SER | A | 1373 | −24.261 | −31.992 | 17.573 | 1.00 | 59.57 | A |
| 2843 | O | SER | A | 1373 | −25.511 | −31.881 | 17.687 | 1.00 | 60.06 | A |
| 2844 | N | GLU | A | 1374 | −23.472 | −31.621 | 18.573 | 1.00 | 59.34 | A |
| 2845 | CA | GLU | A | 1374 | −24.018 | −30.731 | 19.589 | 1.00 | 60.82 | A |
| 2846 | CB | GLU | A | 1374 | −24.154 | −31.368 | 20.982 | 1.00 | 61.04 | A |
| 2847 | CG | GLU | A | 1374 | −25.035 | −32.690 | 20.880 | 1.00 | 62.49 | A |
| 2848 | CD | GLU | A | 1374 | −24.250 | −33.999 | 20.518 | 1.00 | 63.95 | A |
| 2849 | OE1 | GLU | A | 1374 | −23.000 | −33.972 | 20.152 | 1.00 | 56.36 | A |
| 2850 | OE2 | GLU | A | 1374 | −24.909 | −35.084 | 20.635 | 1.00 | 65.88 | A |
| 2851 | C | GLU | A | 1374 | −23.249 | −29.409 | 19.616 | 1.00 | 59.88 | A |
| 2852 | O | GLU | A | 1374 | −23.724 | −28.467 | 20.214 | 1.00 | 62.58 | A |
| 2853 | N | LEU | A | 1375 | −22.097 | −29.325 | 18.957 | 1.00 | 55.89 | A |
| 2854 | CA | LEU | A | 1375 | −21.377 | −28.058 | 18.794 | 1.00 | 50.93 | A |
| 2855 | CB | LEU | A | 1375 | −20.117 | −28.300 | 17.967 | 1.00 | 51.15 | A |
| 2856 | CG | LEU | A | 1375 | −18.690 | −28.543 | 18.604 | 1.00 | 51.01 | A |
| 2857 | CD1 | LEU | A | 1375 | −18.635 | −29.075 | 20.074 | 1.00 | 47.34 | A |
| 2858 | CD2 | LEU | A | 1375 | −17.724 | −29.339 | 17.673 | 1.00 | 46.16 | A |
| 2859 | C | LEU | A | 1375 | −22.158 | −26.860 | 18.128 | 1.00 | 50.72 | A |
| 2860 | O | LEU | A | 1375 | −22.624 | −27.002 | 17.007 | 1.00 | 48.70 | A |
| 2861 | N | PHE | A | 1376 | −22.229 | −25.692 | 18.835 | 1.00 | 48.72 | A |
| 2862 | CA | PHE | A | 1376 | −22.783 | −24.419 | 18.340 | 1.00 | 48.07 | A |
| 2863 | CB | PHE | A | 1376 | −23.713 | −23.767 | 19.352 | 1.00 | 46.36 | A |
| 2864 | CG | PHE | A | 1376 | −24.953 | −24.535 | 19.551 | 1.00 | 50.49 | A |
| 2865 | CD1 | PHE | A | 1376 | −24.893 | −25.837 | 20.110 | 1.00 | 55.68 | A |
| 2866 | CD2 | PHE | A | 1376 | −26.171 | −24.031 | 19.216 | 1.00 | 48.27 | A |
| 2867 | CE1 | PHE | A | 1376 | −26.105 | −26.620 | 20.332 | 1.00 | 50.46 | A |
| 2868 | CE2 | PHE | A | 1376 | −27.352 | −24.775 | 19.471 | 1.00 | 50.20 | A |
| 2869 | CZ | PHE | A | 1376 | −27.319 | −26.070 | 20.043 | 1.00 | 46.59 | A |
| 2870 | C | PHE | A | 1376 | −21.816 | −23.400 | 18.015 | 1.00 | 45.22 | A |
| 2871 | O | PHE | A | 1376 | −20.937 | −23.242 | 18.810 | 1.00 | 49.23 | A |
| 2872 | N | LEU | A | 1377 | −22.116 | −22.605 | 16.969 | 1.00 | 41.02 | A |
| 2873 | CA | LEU | A | 1377 | −21.401 | −21.412 | 16.571 | 1.00 | 37.80 | A |
| 2874 | CB | LEU | A | 1377 | −21.376 | −21.241 | 15.069 | 1.00 | 40.22 | A |
| 2875 | CG | LEU | A | 1377 | −20.891 | −19.863 | 14.705 | 1.00 | 44.81 | A |
| 2876 | CD1 | LEU | A | 1377 | −20.186 | −19.903 | 13.426 | 1.00 | 40.72 | A |
| 2877 | CD2 | LEU | A | 1377 | −21.998 | −18.551 | 14.879 | 1.00 | 44.75 | A |
| 2878 | C | LEU | A | 1377 | −22.079 | −20.280 | 17.122 | 1.00 | 36.28 | A |
| 2879 | O | LEU | A | 1377 | −23.177 | −20.181 | 17.036 | 1.00 | 36.03 | A |
| 2880 | N | MET | A | 1378 | −21.454 | −19.406 | 17.818 | 1.00 | 38.71 | A |
| 2881 | CA | MET | A | 1378 | −22.196 | −18.340 | 18.427 | 1.00 | 37.60 | A |
| 2882 | CB | MET | A | 1378 | −22.174 | −18.537 | 19.846 | 1.00 | 33.48 | A |
| 2883 | CG | MET | A | 1378 | −22.623 | −17.357 | 20.556 | 1.00 | 39.80 | A |
| 2884 | SD | MET | A | 1378 | −22.714 | −17.585 | 22.474 | 1.00 | 39.02 | A |
| 2885 | CE | MET | A | 1378 | −21.038 | −17.139 | 22.750 | 1.00 | 28.85 | A |
| 2886 | C | MET | A | 1378 | −21.424 | −17.051 | 17.942 | 1.00 | 38.09 | A |
| 2887 | O | MET | A | 1378 | −20.171 | −16.964 | 17.906 | 1.00 | 33.46 | A |
| 2888 | N | LYS | A | 1379 | −22.255 | −16.078 | 17.548 | 1.00 | 40.38 | A |
| 2889 | CA | LYS | A | 1379 | −21.903 | −14.796 | 16.858 | 1.00 | 43.90 | A |
| 2890 | CB | LYS | A | 1379 | −22.495 | −14.813 | 15.367 | 1.00 | 45.91 | A |
| 2891 | CG | LYS | A | 1379 | −22.918 | −13.479 | 14.672 | 1.00 | 41.93 | A |
| 2892 | CD | LYS | A | 1379 | −22.212 | −13.455 | 13.330 | 1.00 | 53.38 | A |
| 2893 | CE | LYS | A | 1379 | −22.495 | −12.291 | 12.484 | 1.00 | 50.65 | A |
| 2894 | NZ | LYS | A | 1379 | −23.278 | −12.935 | 11.335 | 1.00 | 50.59 | A |
| 2895 | C | LYS | A | 1379 | −22.537 | −13.635 | 17.620 | 1.00 | 45.00 | A |
| 2896 | O | LYS | A | 1379 | −23.868 | −13.523 | 17.759 | 1.00 | 44.44 | A |
| 2897 | N | LEU | A | 1380 | −21.609 | −12.826 | 18.151 | 1.00 | 45.05 | A |
| 2898 | CA | LEU | A | 1380 | −21.993 | −11.588 | 18.936 | 1.00 | 44.97 | A |
| 2899 | CB | LEU | A | 1380 | −20.727 | −10.914 | 19.384 | 1.00 | 43.50 | A |
| 2900 | CG | LEU | A | 1380 | −20.855 | −9.992 | 20.514 | 1.00 | 40.79 | A |
| 2901 | CD1 | LEU | A | 1380 | −19.627 | −9.272 | 20.552 | 1.00 | 32.67 | A |
| 2902 | CD2 | LEU | A | 1380 | −21.810 | −9.113 | 20.137 | 1.00 | 39.10 | A |
| 2903 | C | LEU | A | 1380 | −22.493 | −10.777 | 17.783 | 1.00 | 43.17 | A |
| 2904 | O | LEU | A | 1380 | −21.780 | −10.794 | 16.816 | 1.00 | 43.12 | A |
| 2905 | N | ILE | A | 1381 | −23.679 | −10.179 | 17.848 | 1.00 | 42.15 | A |
| 2906 | CA | ILE | A | 1381 | −24.169 | −9.337 | 16.739 | 1.00 | 42.48 | A |
| 2907 | CB | ILE | A | 1381 | −25.561 | −9.838 | 16.048 | 1.00 | 42.09 | A |
| 2908 | CG2 | ILE | A | 1381 | −25.314 | −11.144 | 15.494 | 1.00 | 47.74 | A |
| 2909 | CG1 | ILE | A | 1381 | −26.791 | −10.063 | 16.888 | 1.00 | 32.03 | A |
| 2910 | CD1 | ILE | A | 1381 | −27.478 | −8.735 | 17.407 | 1.00 | 28.32 | A |
| 2911 | C | ILE | A | 1381 | −24.378 | −7.880 | 16.905 | 1.00 | 42.12 | A |
| 2912 | O | ILE | A | 1381 | −24.665 | −7.196 | 15.875 | 1.00 | 38.08 | A |
| 2913 | N | ASN | A | 1382 | −24.391 | −7.487 | 18.217 | 1.00 | 41.13 | A |
| 2914 | CA | ASN | A | 1382 | −24.503 | −6.135 | 18.622 | 1.00 | 39.52 | A |
| 2915 | CB | ASN | A | 1382 | −25.546 | −6.037 | 19.687 | 1.00 | 39.27 | A |
| 2916 | CG | ASN | A | 1382 | −25.103 | −6.614 | 21.077 | 1.00 | 45.27 | A |
| 2917 | OD1 | ASN | A | 1382 | −24.073 | −7.418 | 21.223 | 1.00 | 44.38 | A |
| 2918 | ND2 | ASN | A | 1382 | −25.916 | −6.255 | 22.119 | 1.00 | 27.56 | A |
| 2919 | C | ASN | A | 1382 | −23.276 | −5.316 | 18.985 | 1.00 | 39.89 | A |
| 2920 | O | ASN | A | 1382 | −23.441 | −4.268 | 19.703 | 1.00 | 43.98 | A |
| 2921 | N | ARG | A | 1383 | −22.075 | −5.636 | 18.500 | 1.00 | 39.19 | A |
| 2922 | CA | ARG | A | 1383 | −20.851 | −4.809 | 18.822 | 1.00 | 36.86 | A |
| 2923 | CB | ARG | A | 1383 | −19.963 | −5.444 | 19.881 | 1.00 | 35.30 | A |
| 2924 | CG | ARG | A | 1383 | −20.520 | −5.483 | 21.112 | 1.00 | 32.24 | A |
| 2925 | CD | ARG | A | 1383 | −21.164 | −4.022 | 21.391 | 1.00 | 30.14 | A |
| 2926 | NE | ARG | A | 1383 | −20.967 | −3.686 | 22.762 | 1.00 | 33.68 | A |
| 2927 | CZ | ARG | A | 1383 | −21.820 | −3.950 | 23.768 | 1.00 | 36.69 | A |
| 2928 | NH1 | ARG | A | 1383 | −23.121 | −4.249 | 23.468 | 1.00 | 29.53 | A |
| 2929 | NH2 | ARG | A | 1383 | −21.467 | −3.624 | 25.082 | 1.00 | 34.36 | A |
| 2930 | C | ARG | A | 1383 | −19.960 | −4.640 | 17.642 | 1.00 | 37.80 | A |
| 2931 | O | ARG | A | 1383 | −18.841 | −5.000 | 17.687 | 1.00 | 36.84 | A |
| 2932 | N | PRO | A | 1384 | −20.482 | −4.112 | 16.528 | 1.00 | 39.69 | A |
| 2933 | CD | PRO | A | 1384 | −21.777 | −3.512 | 16.215 | 1.00 | 37.34 | A |
| 2934 | CA | PRO | A | 1384 | −19.716 | −4.266 | 15.327 | 1.00 | 38.56 | A |
| 2935 | CB | PRO | A | 1384 | −20.763 | −4.035 | 14.328 | 1.00 | 34.74 | A |
| 2936 | CG | PRO | A | 1384 | −21.560 | −3.219 | 14.928 | 1.00 | 33.26 | A |
| 2937 | C | PRO | A | 1384 | −18.672 | −3.196 | 15.424 | 1.00 | 41.20 | A |
| 2938 | O | PRO | A | 1384 | −17.581 | −3.263 | 14.787 | 1.00 | 41.75 | A |
| 2939 | N | ILE | A | 1385 | −19.024 | −2.257 | 16.318 | 1.00 | 41.94 | A |
| 2940 | CA | ILE | A | 1385 | −18.159 | −1.118 | 16.669 | 1.00 | 43.36 | A |
| 2941 | CB | ILE | A | 1385 | −19.073 | 0.030 | 16.345 | 1.00 | 41.65 | A |
| 2942 | CG2 | ILE | A | 1385 | −19.700 | 0.447 | 17.528 | 1.00 | 46.55 | A |
| 2943 | CG1 | ILE | A | 1385 | −18.439 | 1.343 | 16.001 | 1.00 | 47.51 | A |
| 2944 | CD1 | ILE | A | 1385 | −19.625 | 2.647 | 16.196 | 1.00 | 40.88 | A |
| 2945 | C | ILE | A | 1385 | −17.944 | −1.376 | 18.280 | 1.00 | 42.53 | A |
| 2946 | O | ILE | A | 1385 | −18.972 | −1.439 | 18.965 | 1.00 | 40.52 | A |
| 2947 | N | ILE | A | 1386 | −16.739 | −1.627 | 18.839 | 1.00 | 41.78 | A |
| 2948 | CA | ILE | A | 1386 | −16.537 | −1.995 | 20.362 | 1.00 | 42.59 | A |
| 2949 | CB | ILE | A | 1386 | −16.050 | −3.541 | 20.709 | 1.00 | 45.19 | A |
| 2950 | CG2 | ILE | A | 1386 | −17.013 | −4.336 | 21.980 | 1.00 | 40.21 | A |
| 2951 | CG1 | ILE | A | 1386 | −15.779 | −4.349 | 19.441 | 1.00 | 41.40 | A |
| 2952 | CD1 | ILE | A | 1386 | −14.726 | −3.789 | 18.775 | 1.00 | 45.03 | A |
| 2953 | C | ILE | A | 1386 | −15.459 | −1.177 | 21.117 | 1.00 | 41.86 | A |
| 2954 | O | ILE | A | 1386 | −14.618 | −0.543 | 20.514 | 1.00 | 41.93 | A |
| 2955 | N | VAL | A | 1387 | −15.427 | −1.245 | 22.441 | 1.00 | 42.02 | A |
| 2956 | CA | VAL | A | 1387 | −14.409 | −0.485 | 23.282 | 1.00 | 39.92 | A |
| 2957 | CB | VAL | A | 1387 | −14.914 | 0.927 | 23.623 | 1.00 | 40.61 | A |
| 2958 | CG1 | VAL | A | 1387 | −13.816 | 1.749 | 24.445 | 1.00 | 44.19 | A |
| 2959 | CG2 | VAL | A | 1387 | −15.175 | 1.640 | 22.175 | 1.00 | 34.79 | A |
| 2960 | C | VAL | A | 1387 | −14.169 | −1.383 | 24.421 | 1.00 | 37.51 | A |
| 2961 | O | VAL | A | 1387 | −15.115 | −1.992 | 24.795 | 1.00 | 37.27 | A |
| 2962 | N | PHE | A | 1388 | −12.961 | −1.532 | 24.916 | 1.00 | 36.60 | A |
| 2963 | CA | PHE | A | 1388 | −12.661 | −2.626 | 25.950 | 1.00 | 38.89 | A |
| 2964 | CB | PHE | A | 1388 | −11.507 | −3.597 | 25.495 | 1.00 | 41.04 | A |
| 2965 | CG | PHE | A | 1388 | −11.807 | −4.344 | 24.201 | 1.00 | 40.99 | A |
| 2966 | CD1 | PHE | A | 1388 | −12.662 | −5.475 | 24.213 | 1.00 | 39.56 | A |
| 2967 | CD2 | PHE | A | 1388 | −11.499 | −3.728 | 22.916 | 1.00 | 45.41 | A |
| 2968 | CE1 | PHE | A | 1388 | −12.970 | −6.214 | 23.009 | 1.00 | 36.39 | A |
| 2969 | CE2 | PHE | A | 1388 | −11.788 | −4.421 | 21.632 | 1.00 | 40.60 | A |
| 2970 | CZ | PHE | A | 1388 | −12.516 | −5.701 | 21.742 | 1.00 | 42.12 | A |
| 2971 | C | PHE | A | 1388 | −12.154 | −1.980 | 27.071 | 1.00 | 38.56 | A |
| 2972 | O | PHE | A | 1388 | −11.235 | −1.241 | 26.953 | 1.00 | 44.46 | A |
| 2973 | N | ARG | A | 1389 | −12.858 | −2.030 | 28.128 | 1.00 | 38.79 | A |
| 2974 | CA | ARG | A | 1389 | −12.418 | −1.473 | 29.428 | 1.00 | 38.76 | A |
| 2975 | CB | ARG | A | 1389 | −13.558 | −0.889 | 30.124 | 1.00 | 37.37 | A |
| 2976 | CG | ARG | A | 1389 | −13.031 | 0.046 | 31.281 | 1.00 | 39.11 | A |
| 2977 | CD | ARG | A | 1389 | −14.232 | 0.265 | 32.150 | 1.00 | 42.37 | A |
| 2978 | NE | ARG | A | 1389 | −14.315 | 1.585 | 32.575 | 1.00 | 43.25 | A |
| 2979 | CZ | ARG | A | 1389 | −15.217 | 2.031 | 33.454 | 1.00 | 39.73 | A |
| 2980 | NH1 | ARG | A | 1389 | −15.952 | 1.172 | 33.990 | 1.00 | 35.19 | A |
| 2981 | NH2 | ARG | A | 1389 | −15.327 | 3.331 | 33.809 | 1.00 | 45.09 | A |
| 2982 | C | ARG | A | 1389 | −12.022 | −2.479 | 30.456 | 1.00 | 40.28 | A |
| 2983 | O | ARG | A | 1389 | −12.921 | −3.213 | 30.859 | 1.00 | 36.19 | A |
| 2984 | N | GLY | A | 1390 | −10.677 | −2.569 | 30.716 | 1.00 | 43.57 | A |
| 2985 | CA | GLY | A | 1390 | −10.002 | −3.327 | 31.799 | 1.00 | 47.33 | A |
| 2986 | C | GLY | A | 1390 | −10.238 | −2.811 | 33.276 | 1.00 | 49.93 | A |
| 2987 | O | GLY | A | 1390 | −11.285 | −2.935 | 33.794 | 1.00 | 50.03 | A |
| 2988 | N | GLU | A | 1391 | −9.311 | −2.157 | 33.893 | 1.00 | 53.45 | A |
| 2989 | CA | GLU | A | 1391 | −9.013 | −2.226 | 35.398 | 1.00 | 56.77 | A |
| 2990 | CB | GLU | A | 1391 | −7.758 | −3.095 | 35.556 | 1.00 | 58.18 | A |
| 2991 | CG | GLU | A | 1391 | −8.139 | −4.533 | 35.658 | 1.00 | 62.62 | A |
| 2992 | CD | GLU | A | 1391 | −7.448 | −5.338 | 36.855 | 1.00 | 64.88 | A |
| 2993 | OE1 | GLU | A | 1391 | −6.168 | −5.615 | 36.842 | 1.00 | 60.83 | A |
| 2994 | OE2 | GLU | A | 1391 | −8.253 | −5.732 | 37.767 | 1.00 | 58.76 | A |
| 2995 | C | GLU | A | 1391 | −8.558 | −0.830 | 35.931 | 1.00 | 57.24 | A |
| 2996 | O | GLU | A | 1391 | −9.247 | −0.202 | 36.639 | 1.00 | 59.18 | A |
| 2997 | N | HIS | A | 1392 | −7.369 | −0.360 | 35.525 | 1.00 | 57.11 | A |
| 2998 | CA | HIS | A | 1392 | −7.042 | 1.066 | 35.528 | 1.00 | 56.66 | A |
| 2999 | CB | HIS | A | 1392 | −5.745 | 1.325 | 36.453 | 1.00 | 59.76 | A |
| 3000 | CG | HIS | A | 1392 | −5.502 | 0.184 | 37.406 | 1.00 | 63.94 | A |
| 3001 | CD2 | HIS | A | 1392 | −4.603 | −0.836 | 37.324 | 1.00 | 65.69 | A |
| 3002 | ND1 | HIS | A | 1392 | −6.432 | −0.181 | 38.388 | 1.00 | 57.85 | A |
| 3003 | CE1 | HIS | A | 1392 | −6.106 | −1.376 | 38.840 | 1.00 | 62.73 | A |
| 3004 | NE2 | HIS | A | 1392 | −4.952 | −1.550 | 38.243 | 1.00 | 63.71 | A |
| 3005 | C | HIS | A | 1392 | −6.907 | 1.498 | 34.023 | 1.00 | 55.22 | A |
| 3006 | O | HIS | A | 1392 | −5.787 | 1.848 | 33.583 | 1.00 | 54.00 | A |
| 3007 | N | GLY | A | 1393 | −8.019 | 1.331 | 33.244 | 1.00 | 52.49 | A |
| 3008 | CA | GLY | A | 1393 | −8.392 | 2.092 | 32.026 | 1.00 | 50.24 | A |
| 3009 | C | GLY | A | 1393 | −8.917 | 1.420 | 30.742 | 1.00 | 49.56 | A |
| 3010 | O | GLY | A | 1393 | −9.255 | 0.198 | 30.650 | 1.00 | 52.43 | A |
| 3011 | N | PHE | A | 1394 | −8.852 | 2.131 | 29.658 | 1.00 | 46.49 | A |
| 3012 | CA | PHE | A | 1394 | −9.414 | 1.525 | 28.464 | 1.00 | 45.59 | A |
| 3013 | CB | PHE | A | 1394 | −10.171 | 2.561 | 27.601 | 1.00 | 43.86 | A |
| 3014 | CG | PHE | A | 1394 | −11.489 | 3.014 | 28.269 | 1.00 | 37.61 | A |
| 3015 | CD1 | PHE | A | 1394 | −11.520 | 4.169 | 28.958 | 1.00 | 27.24 | A |
| 3016 | CD2 | PHE | A | 1394 | −12.623 | 2.203 | 28.267 | 1.00 | 34.34 | A |
| 3017 | CE1 | PHE | A | 1394 | −12.587 | 4.590 | 29.616 | 1.00 | 30.36 | A |
| 3018 | CE2 | PHE | A | 1394 | −13.799 | 2.681 | 28.810 | 1.00 | 35.30 | A |
| 3019 | CZ | PHE | A | 1394 | −13.801 | 3.892 | 29.489 | 1.00 | 39.72 | A |
| 3020 | C | PHE | A | 1394 | −8.420 | 0.775 | 27.680 | 1.00 | 46.38 | A |
| 3021 | O | PHE | A | 1394 | −7.190 | 1.075 | 27.819 | 1.00 | 49.95 | A |
| 3022 | N | ILE | A | 1395 | −8.879 | −0.142 | 26.834 | 1.00 | 44.41 | A |
| 3023 | CA | ILE | A | 1395 | −7.984 | −0.512 | 25.745 | 1.00 | 44.34 | A |
| 3024 | CB | ILE | A | 1395 | −8.258 | −1.849 | 25.191 | 1.00 | 44.43 | A |
| 3025 | CG2 | ILE | A | 1395 | −7.171 | −2.287 | 24.321 | 1.00 | 44.81 | A |
| 3026 | CG1 | ILE | A | 1395 | −8.457 | −2.964 | 26.273 | 1.00 | 45.92 | A |
| 3027 | CD1 | ILE | A | 1395 | −8.503 | −4.542 | 25.541 | 1.00 | 50.31 | A |
| 3028 | C | ILE | A | 1395 | −7.871 | 0.564 | 24.669 | 1.00 | 43.95 | A |
| 3029 | O | ILE | A | 1395 | −8.747 | 1.324 | 24.356 | 1.00 | 42.42 | A |
| 3030 | N | GLY | A | 1396 | −6.683 | 0.648 | 24.172 | 1.00 | 47.46 | A |
| 3031 | CA | GLY | A | 1396 | −6.192 | 1.804 | 23.410 | 1.00 | 51.27 | A |
| 3032 | C | GLY | A | 1396 | −4.799 | 1.592 | 22.792 | 1.00 | 53.85 | A |
| 3033 | O | GLY | A | 1396 | −3.893 | 1.016 | 23.388 | 1.00 | 56.79 | A |
| 3034 | N | CYS | A | 1397 | −4.598 | 2.031 | 21.573 | 1.00 | 55.64 | A |
| 3035 | CA | CYS | A | 1397 | −3.307 | 1.860 | 21.017 | 1.00 | 57.85 | A |
| 3036 | CB | CYS | A | 1397 | −3.243 | 2.153 | 19.478 | 1.00 | 56.69 | A |
| 3037 | SG | CYS | A | 1397 | −4.889 | 1.812 | 18.758 | 1.00 | 65.91 | A |
| 3038 | C | CYS | A | 1397 | −2.562 | 2.935 | 21.720 | 1.00 | 57.37 | A |
| 3039 | O | CYS | A | 1397 | −3.050 | 4.077 | 22.022 | 1.00 | 54.23 | A |
| 3040 | N | ARG | A | 1398 | −1.328 | 2.536 | 21.872 | 1.00 | 58.80 | A |
| 3041 | CA | ARG | A | 1398 | −0.274 | 3.475 | 21.743 | 1.00 | 59.63 | A |
| 3042 | CB | ARG | A | 1398 | 1.030 | 2.812 | 21.483 | 1.00 | 58.62 | A |
| 3043 | CG | ARG | A | 1398 | 2.198 | 3.498 | 22.273 | 1.00 | 62.50 | A |
| 3044 | CD | ARG | A | 1398 | 2.091 | 3.518 | 23.780 | 1.00 | 65.92 | A |
| 3045 | NE | ARG | A | 1398 | 2.406 | 2.212 | 24.346 | 1.00 | 68.01 | A |
| 3046 | CZ | ARG | A | 1398 | 2.353 | 1.874 | 25.653 | 1.00 | 75.31 | A |
| 3047 | NH1 | ARG | A | 1398 | 1.998 | 2.720 | 26.639 | 1.00 | 73.15 | A |
| 3048 | NH2 | ARG | A | 1398 | 2.657 | 0.623 | 26.019 | 1.00 | 81.89 | A |
| 3049 | C | ARG | A | 1398 | −0.509 | 4.703 | 20.838 | 1.00 | 60.42 | A |
| 3050 | O | ARG | A | 1398 | −1.609 | 5.246 | 20.702 | 1.00 | 58.64 | A |
| 3051 | N | LYS | A | 1399 | 0.611 | 5.275 | 20.470 | 1.00 | 63.88 | A |
| 3052 | CA | LYS | A | 1399 | 0.690 | 6.620 | 19.923 | 1.00 | 64.03 | A |
| 3053 | CB | LYS | A | 1399 | 1.254 | 7.666 | 20.946 | 1.00 | 64.69 | A |
| 3054 | CG | LYS | A | 1399 | 1.305 | 9.322 | 20.357 | 1.00 | 64.27 | A |
| 3055 | CD | LYS | A | 1399 | 2.509 | 10.409 | 20.738 | 1.00 | 59.24 | A |
| 3056 | CE | LYS | A | 1399 | 4.076 | 9.883 | 20.910 | 1.00 | 54.47 | A |
| 3057 | NZ | LYS | A | 1399 | 5.076 | 11.101 | 21.302 | 1.00 | 60.13 | A |
| 3058 | C | LYS | A | 1399 | 1.530 | 6.431 | 18.590 | 1.00 | 64.70 | A |
| 3059 | O | LYS | A | 1399 | 0.934 | 6.332 | 17.485 | 1.00 | 67.48 | A |
| 3060 | N | VAL | A | 1400 | 2.839 | 6.255 | 18.679 | 1.00 | 62.52 | A |
| 3061 | CA | VAL | A | 1400 | 3.602 | 5.989 | 17.536 | 1.00 | 61.69 | A |
| 3062 | CB | VAL | A | 1400 | 4.418 | 7.297 | 17.125 | 1.00 | 64.66 | A |
| 3063 | CG1 | VAL | A | 1400 | 4.885 | 7.315 | 15.487 | 1.00 | 65.86 | A |
| 3064 | CG2 | VAL | A | 1400 | 3.634 | 8.678 | 17.572 | 1.00 | 61.99 | A |
| 3065 | C | VAL | A | 1400 | 4.484 | 4.776 | 17.754 | 1.00 | 61.76 | A |
| 3066 | O | VAL | A | 1400 | 5.575 | 4.741 | 17.205 | 1.00 | 62.66 | A |
| 3067 | N | THR | A | 1401 | 4.016 | 3.778 | 18.548 | 1.00 | 61.99 | A |
| 3068 | CA | THR | A | 1401 | 4.499 | 2.298 | 18.521 | 1.00 | 59.76 | A |
| 3069 | CB | THR | A | 1401 | 4.969 | 1.804 | 20.022 | 1.00 | 60.83 | A |
| 3070 | OG1 | THR | A | 1401 | 6.462 | 1.823 | 20.260 | 1.00 | 55.71 | A |
| 3071 | CG2 | THR | A | 1401 | 4.230 | 0.409 | 20.378 | 1.00 | 53.54 | A |
| 3072 | C | THR | A | 1401 | 3.560 | 1.154 | 17.849 | 1.00 | 59.89 | A |
| 3073 | O | THR | A | 1401 | 3.993 | 0.159 | 17.251 | 1.00 | 60.51 | A |
| 3074 | N | GLY | A | 1402 | 2.265 | 1.243 | 17.980 | 1.00 | 57.87 | A |
| 3075 | CA | GLY | A | 1402 | 1.576 | 0.119 | 17.576 | 1.00 | 57.98 | A |
| 3076 | C | GLY | A | 1402 | 0.889 | −0.397 | 18.796 | 1.00 | 57.80 | A |
| 3077 | O | GLY | A | 1402 | −0.306 | −0.702 | 18.714 | 1.00 | 59.21 | A |
| 3078 | N | THR | A | 1403 | 1.592 | −0.378 | 19.942 | 1.00 | 57.69 | A |
| 3079 | CA | THR | A | 1403 | 1.298 | −1.360 | 21.048 | 1.00 | 53.83 | A |
| 3080 | CB | THR | A | 1403 | 2.400 | −1.620 | 22.101 | 1.00 | 52.23 | A |
| 3081 | OG1 | THR | A | 1403 | 3.673 | −1.786 | 21.443 | 1.00 | 54.49 | A |
| 3082 | CG2 | THR | A | 1403 | 2.134 | −2.880 | 22.618 | 1.00 | 44.58 | A |
| 3083 | C | THR | A | 1403 | −0.014 | −1.187 | 21.672 | 1.00 | 52.84 | A |
| 3084 | O | THR | A | 1403 | −0.520 | −0.014 | 21.866 | 1.00 | 51.18 | A |
| 3085 | N | LEU | A | 1404 | −0.637 | −2.339 | 21.859 | 1.00 | 51.28 | A |
| 3086 | CA | LEU | A | 1404 | −1.815 | −2.254 | 22.524 | 1.00 | 52.39 | A |
| 3087 | CB | LEU | A | 1404 | −2.794 | −3.330 | 22.136 | 1.00 | 52.14 | A |
| 3088 | CG | LEU | A | 1404 | −3.644 | −3.234 | 20.813 | 1.00 | 46.24 | A |
| 3089 | CD1 | LEU | A | 1404 | −4.237 | −1.922 | 20.423 | 1.00 | 42.27 | A |
| 3090 | CD2 | LEU | A | 1404 | −2.953 | −3.781 | 19.614 | 1.00 | 47.59 | A |
| 3091 | C | LEU | A | 1404 | −1.666 | −1.915 | 24.038 | 1.00 | 55.39 | A |
| 3092 | O | LEU | A | 1404 | −0.855 | −2.486 | 24.719 | 1.00 | 57.16 | A |
| 3093 | N | ASP | A | 1405 | −2.341 | −0.842 | 24.502 | 1.00 | 57.13 | A |
| 3094 | CA | ASP | A | 1405 | −2.455 | −0.523 | 25.904 | 1.00 | 56.29 | A |
| 3095 | CB | ASP | A | 1405 | −2.743 | 0.923 | 25.986 | 1.00 | 57.29 | A |
| 3096 | CG | ASP | A | 1405 | −1.511 | 1.693 | 26.209 | 1.00 | 66.43 | A |
| 3097 | OD1 | ASP | A | 1405 | −0.693 | 1.710 | 25.225 | 1.00 | 77.55 | A |
| 3098 | OD2 | ASP | A | 1405 | −1.309 | 2.196 | 27.376 | 1.00 | 67.72 | A |
| 3099 | C | ASP | A | 1405 | −3.555 | −1.243 | 26.704 | 1.00 | 55.00 | A |
| 3100 | O | ASP | A | 1405 | −4.619 | −1.506 | 26.203 | 1.00 | 54.88 | A |
| 3101 | N | ALA | A | 1406 | −3.312 | −1.504 | 27.978 | 1.00 | 53.42 | A |
| 3102 | CA | ALA | A | 1406 | −4.373 | −1.965 | 28.780 | 1.00 | 53.18 | A |
| 3103 | CB | ALA | A | 1406 | −3.923 | −3.028 | 29.642 | 1.00 | 51.53 | A |
| 3104 | C | ALA | A | 1406 | −4.958 | −0.888 | 29.597 | 1.00 | 54.00 | A |
| 3105 | O | ALA | A | 1406 | −5.854 | −1.148 | 30.420 | 1.00 | 56.14 | A |
| 3106 | N | ASN | A | 1407 | −4.453 | 0.318 | 29.461 | 1.00 | 53.28 | A |
| 3107 | CA | ASN | A | 1407 | −4.733 | 1.227 | 30.523 | 1.00 | 53.63 | A |
| 3108 | CB | ASN | A | 1407 | −3.683 | 1.020 | 31.575 | 1.00 | 54.49 | A |
| 3109 | CG | ASN | A | 1407 | −2.374 | 1.678 | 31.198 | 1.00 | 54.66 | A |
| 3110 | OD1 | ASN | A | 1407 | −2.355 | 2.742 | 30.630 | 1.00 | 57.87 | A |
| 3111 | ND2 | ASN | A | 1407 | −1.251 | 1.013 | 31.506 | 1.00 | 55.66 | A |
| 3112 | C | ASN | A | 1407 | −4.734 | 2.703 | 30.169 | 1.00 | 54.57 | A |
| 3113 | O | ASN | A | 1407 | −4.278 | 3.572 | 30.979 | 1.00 | 53.32 | A |
| 3114 | N | ARG | A | 1408 | −5.206 | 3.029 | 28.975 | 1.00 | 54.11 | A |
| 3115 | CA | ARG | A | 1408 | −4.997 | 4.372 | 28.588 | 1.00 | 54.32 | A |
| 3116 | CB | ARG | A | 1408 | −4.973 | 4.435 | 27.064 | 1.00 | 55.69 | A |
| 3117 | CG | ARG | A | 1408 | −4.066 | 3.455 | 26.442 | 1.00 | 53.69 | A |
| 3118 | CD | ARG | A | 1408 | −3.541 | 3.857 | 24.936 | 1.00 | 57.92 | A |
| 3119 | NE | ARG | A | 1408 | −3.167 | 5.242 | 24.597 | 1.00 | 51.71 | A |
| 3120 | CZ | ARG | A | 1408 | −4.087 | 6.079 | 24.208 | 1.00 | 48.26 | A |
| 3121 | NH1 | ARG | A | 1408 | −3.776 | 7.313 | 23.920 | 1.00 | 38.43 | A |
| 3122 | NH2 | ARG | A | 1408 | −5.324 | 5.632 | 24.181 | 1.00 | 44.81 | A |
| 3123 | C | ARG | A | 1408 | −6.202 | 5.001 | 29.276 | 1.00 | 52.91 | A |
| 3124 | O | ARG | A | 1408 | −6.972 | 4.217 | 29.876 | 1.00 | 52.61 | A |
| 3125 | N | SER | A | 1409 | −6.383 | 6.328 | 29.215 | 1.00 | 51.70 | A |
| 3126 | CA | SER | A | 1409 | −7.515 | 7.066 | 30.013 | 1.00 | 53.20 | A |
| 3127 | CB | SER | A | 1409 | −6.906 | 8.303 | 30.624 | 1.00 | 49.62 | A |
| 3128 | OG | SER | A | 1409 | −6.370 | 8.882 | 29.468 | 1.00 | 58.82 | A |
| 3129 | C | SER | A | 1409 | −8.889 | 7.334 | 29.204 | 1.00 | 50.11 | A |
| 3130 | O | SER | A | 1409 | −9.942 | 7.997 | 29.579 | 1.00 | 46.60 | A |
| 3131 | N | SER | A | 1410 | −8.844 | 6.733 | 28.050 | 1.00 | 50.98 | A |
| 3132 | CA | SER | A | 1410 | −9.700 | 7.194 | 26.921 | 1.00 | 50.18 | A |
| 3133 | CB | SER | A | 1410 | −9.147 | 8.432 | 26.245 | 1.00 | 48.65 | A |
| 3134 | OG | SER | A | 1410 | −9.794 | 9.550 | 26.770 | 1.00 | 45.04 | A |
| 3135 | C | SER | A | 1410 | −9.692 | 6.025 | 25.953 | 1.00 | 50.85 | A |
| 3136 | O | SER | A | 1410 | −8.574 | 5.462 | 25.628 | 1.00 | 47.73 | A |
| 3137 | N | TYR | A | 1411 | −10.957 | 5.723 | 25.576 | 1.00 | 49.51 | A |
| 3138 | CA | TYR | A | 1411 | −11.429 | 4.610 | 24.850 | 1.00 | 47.90 | A |
| 3139 | CB | TYR | A | 1411 | −12.806 | 4.303 | 25.388 | 1.00 | 46.95 | A |
| 3140 | CG | TYR | A | 1411 | −13.804 | 5.541 | 25.462 | 1.00 | 49.28 | A |
| 3141 | CD1 | TYR | A | 1411 | −14.183 | 6.311 | 24.319 | 1.00 | 47.47 | A |
| 3142 | CE1 | TYR | A | 1411 | −15.056 | 7.322 | 24.447 | 1.00 | 52.92 | A |
| 3143 | CD2 | TYR | A | 1411 | −14.448 | 5.823 | 26.637 | 1.00 | 47.69 | A |
| 3144 | CE2 | TYR | A | 1411 | −15.342 | 6.906 | 26.823 | 1.00 | 44.51 | A |
| 3145 | CZ | TYR | A | 1411 | −15.656 | 7.684 | 25.770 | 1.00 | 51.14 | A |
| 3146 | OH | TYR | A | 1411 | −16.713 | 8.687 | 25.945 | 1.00 | 38.52 | A |
| 3147 | C | TYR | A | 1411 | −11.507 | 4.853 | 23.363 | 1.00 | 48.38 | A |
| 3148 | O | TYR | A | 1411 | −12.553 | 5.266 | 22.796 | 1.00 | 51.63 | A |
| 3149 | N | ASP | A | 1412 | −10.424 | 4.536 | 22.704 | 1.00 | 47.64 | A |
| 3150 | CA | ASP | A | 1412 | −10.428 | 4.020 | 21.295 | 1.00 | 46.55 | A |
| 3151 | CB | ASP | A | 1412 | −9.089 | 3.408 | 21.065 | 1.00 | 49.73 | A |
| 3152 | CG | ASP | A | 1412 | −7.980 | 4.463 | 20.986 | 1.00 | 51.41 | A |
| 3153 | OD1 | ASP | A | 1412 | −7.187 | 4.317 | 20.092 | 1.00 | 60.54 | A |
| 3154 | OD2 | ASP | A | 1412 | −7.846 | 5.404 | 21.773 | 1.00 | 54.02 | A |
| 3155 | C | ASP | A | 1412 | −11.511 | 3.103 | 20.769 | 1.00 | 46.25 | A |
| 3156 | O | ASP | A | 1412 | −11.814 | 2.069 | 21.358 | 1.00 | 49.69 | A |
| 3157 | N | VAL | A | 1413 | −12.184 | 3.501 | 19.691 | 1.00 | 45.90 | A |
| 3158 | CA | VAL | A | 1413 | −13.340 | 2.677 | 19.034 | 1.00 | 42.01 | A |
| 3159 | CB | VAL | A | 1413 | −14.464 | 3.497 | 18.458 | 1.00 | 37.35 | A |
| 3160 | CG1 | VAL | A | 1413 | −15.666 | 2.859 | 18.616 | 1.00 | 31.40 | A |
| 3161 | CG2 | VAL | A | 1413 | −14.658 | 4.645 | 19.266 | 1.00 | 35.27 | A |
| 3162 | C | VAL | A | 1413 | −12.776 | 1.780 | 17.950 | 1.00 | 44.36 | A |
| 3163 | O | VAL | A | 1413 | −11.957 | 2.271 | 17.045 | 1.00 | 45.80 | A |
| 3164 | N | PHE | A | 1414 | −13.031 | 0.468 | 18.178 | 1.00 | 45.45 | A |
| 3165 | CA | PHE | A | 1414 | −12.605 | −0.611 | 17.298 | 1.00 | 46.30 | A |
| 3166 | CB | PHE | A | 1414 | −11.938 | −1.792 | 18.033 | 1.00 | 42.88 | A |
| 3167 | CG | PHE | A | 1414 | −10.616 | −1.461 | 18.507 | 1.00 | 44.57 | A |
| 3168 | CD1 | PHE | A | 1414 | −9.661 | −1.101 | 17.553 | 1.00 | 41.91 | A |
| 3169 | CD2 | PHE | A | 1414 | −10.286 | −1.359 | 19.959 | 1.00 | 32.91 | A |
| 3170 | CE1 | PHE | A | 1414 | −8.379 | −0.649 | 18.015 | 1.00 | 44.10 | A |
| 3171 | CE2 | PHE | A | 1414 | −8.949 | −0.945 | 20.354 | 1.00 | 35.71 | A |
| 3172 | CZ | PHE | A | 1414 | −8.039 | −0.583 | 19.417 | 1.00 | 37.20 | A |
| 3173 | C | PHE | A | 1414 | −13.780 | −1.137 | 16.530 | 1.00 | 48.20 | A |
| 3174 | O | PHE | A | 1414 | −14.890 | −1.224 | 17.099 | 1.00 | 49.41 | A |
| 3175 | N | GLN | A | 1415 | −13.498 | −1.605 | 15.312 | 1.00 | 47.55 | A |
| 3176 | CA | GLN | A | 1415 | −14.416 | −2.542 | 14.767 | 1.00 | 49.28 | A |
| 3177 | CB | GLN | A | 1415 | −15.020 | −1.875 | 13.493 | 1.00 | 50.13 | A |
| 3178 | CG | GLN | A | 1415 | −14.114 | −1.864 | 12.284 | 1.00 | 53.96 | A |
| 3179 | CD | GLN | A | 1415 | −14.031 | −0.483 | 11.626 | 1.00 | 54.78 | A |
| 3180 | OE1 | GLN | A | 1415 | −12.961 | 0.099 | 11.665 | 1.00 | 55.88 | A |
| 3181 | NE2 | GLN | A | 1415 | −15.143 | 0.041 | 11.036 | 1.00 | 43.53 | A |
| 3182 | C | GLN | A | 1415 | −14.120 | −4.150 | 14.778 | 1.00 | 48.22 | A |
| 3183 | O | GLN | A | 1415 | −13.046 | −4.614 | 15.070 | 1.00 | 45.53 | A |
| 3184 | N | LEU | A | 1416 | −15.085 | −4.990 | 14.422 | 1.00 | 48.46 | A |
| 3185 | CA | LEU | A | 1416 | −15.006 | −6.358 | 14.814 | 1.00 | 48.37 | A |
| 3186 | CB | LEU | A | 1416 | −15.746 | −6.410 | 16.145 | 1.00 | 49.02 | A |
| 3187 | CG | LEU | A | 1416 | −16.466 | −7.317 | 17.109 | 1.00 | 49.16 | A |
| 3188 | CD1 | LEU | A | 1416 | −17.299 | −8.263 | 16.356 | 1.00 | 58.06 | A |
| 3189 | CD2 | LEU | A | 1416 | −15.314 | −7.977 | 17.719 | 1.00 | 53.63 | A |
| 3190 | C | LEU | A | 1416 | −15.780 | −7.073 | 13.757 | 1.00 | 49.15 | A |
| 3191 | O | LEU | A | 1416 | −16.932 | −6.889 | 13.665 | 1.00 | 48.25 | A |
| 3192 | N | GLU | A | 1417 | −15.089 | −7.830 | 12.904 | 1.00 | 50.70 | A |
| 3193 | CA | GLU | A | 1417 | −15.752 | −8.747 | 11.982 | 1.00 | 48.83 | A |
| 3194 | CB | GLU | A | 1417 | −15.107 | −8.857 | 10.563 | 1.00 | 47.46 | A |
| 3195 | CG | GLU | A | 1417 | −13.766 | −8.649 | 10.361 | 1.00 | 47.67 | A |
| 3196 | CD | GLU | A | 1417 | −13.467 | −7.377 | 9.548 | 1.00 | 48.25 | A |
| 3197 | OE1 | GLU | A | 1417 | −12.506 | −7.335 | 8.740 | 1.00 | 47.35 | A |
| 3198 | OE2 | GLU | A | 1417 | −14.072 | −6.345 | 9.760 | 1.00 | 43.04 | A |
| 3199 | C | GLU | A | 1417 | −15.831 | −10.078 | 12.436 | 1.00 | 48.09 | A |
| 3200 | O | GLU | A | 1417 | −14.888 | −10.605 | 12.737 | 1.00 | 50.19 | A |
| 3201 | N | PHE | A | 1418 | −16.957 | −10.724 | 12.229 | 1.00 | 50.97 | A |
| 3202 | CA | PHE | A | 1418 | −17.060 | −12.120 | 12.522 | 1.00 | 51.40 | A |
| 3203 | CB | PHE | A | 1418 | −18.446 | −12.513 | 12.676 | 1.00 | 48.90 | A |
| 3204 | CG | PHE | A | 1418 | −18.557 | −13.812 | 13.454 | 1.00 | 54.09 | A |
| 3205 | CD1 | PHE | A | 1418 | −17.898 | −13.944 | 14.738 | 1.00 | 51.99 | A |
| 3206 | CD2 | PHE | A | 1418 | −19.286 | −14.851 | 12.992 | 1.00 | 52.06 | A |
| 3207 | CE1 | PHE | A | 1418 | −18.007 | −15.061 | 15.467 | 1.00 | 49.04 | A |
| 3208 | CE2 | PHE | A | 1418 | −19.412 | −16.074 | 13.814 | 1.00 | 55.11 | A |
| 3209 | CZ | PHE | A | 1418 | −18.748 | −16.203 | 14.971 | 1.00 | 47.77 | A |
| 3210 | C | PHE | A | 1418 | −16.612 | −13.105 | 11.498 | 1.00 | 53.47 | A |
| 3211 | O | PHE | A | 1418 | −16.803 | −12.919 | 10.292 | 1.00 | 54.29 | A |
| 3212 | N | ASN | A | 1419 | −16.246 | −14.287 | 12.001 | 1.00 | 55.96 | A |
| 3213 | CA | ASN | A | 1419 | −15.714 | −15.351 | 11.129 | 1.00 | 54.97 | A |
| 3214 | CB | ASN | A | 1419 | −14.426 | −14.803 | 10.625 | 1.00 | 53.96 | A |
| 3215 | CG | ASN | A | 1419 | −13.768 | −15.671 | 9.639 | 1.00 | 58.56 | A |
| 3216 | OD1 | ASN | A | 1419 | −14.368 | −16.204 | 8.686 | 1.00 | 61.83 | A |
| 3217 | ND2 | ASN | A | 1419 | −12.472 | −15.756 | 9.801 | 1.00 | 60.97 | A |
| 3218 | C | ASN | A | 1419 | −15.563 | −16.740 | 11.776 | 1.00 | 53.54 | A |
| 3219 | O | ASN | A | 1419 | −14.483 | −17.081 | 12.214 | 1.00 | 53.40 | A |
| 3220 | N | ASP | A | 1420 | −16.703 | −17.492 | 11.883 | 1.00 | 51.85 | A |
| 3221 | CA | ASP | A | 1420 | −16.686 | −18.944 | 12.143 | 1.00 | 49.02 | A |
| 3222 | CB | ASP | A | 1420 | −15.592 | −19.623 | 11.195 | 1.00 | 49.36 | A |
| 3223 | CG | ASP | A | 1420 | −15.494 | −21.171 | 11.355 | 1.00 | 49.16 | A |
| 3224 | OD1 | ASP | A | 1420 | −16.414 | −21.862 | 11.845 | 1.00 | 42.25 | A |
| 3225 | OD2 | ASP | A | 1420 | −14.470 | −21.727 | 10.994 | 1.00 | 52.11 | A |
| 3226 | C | ASP | A | 1420 | −16.240 | −19.058 | 13.543 | 1.00 | 47.72 | A |
| 3227 | O | ASP | A | 1420 | −15.102 | −19.468 | 13.746 | 1.00 | 50.48 | A |
| 3228 | N | GLY | A | 1421 | −17.045 | −18.660 | 14.523 | 1.00 | 45.62 | A |
| 3229 | CA | GLY | A | 1421 | −16.549 | −18.651 | 15.947 | 1.00 | 43.81 | A |
| 3230 | C | GLY | A | 1421 | −15.367 | −17.804 | 16.431 | 1.00 | 42.51 | A |
| 3231 | O | GLY | A | 1421 | −15.296 | −17.544 | 17.582 | 1.00 | 42.31 | A |
| 3232 | N | ALA | A | 1422 | −14.450 | −17.407 | 15.512 | 1.00 | 43.57 | A |
| 3233 | CA | ALA | A | 1422 | −13.479 | −16.273 | 15.661 | 1.00 | 43.56 | A |
| 3234 | CB | ALA | A | 1422 | −12.304 | −16.575 | 14.829 | 1.00 | 44.75 | A |
| 3235 | C | ALA | A | 1422 | −13.864 | −14.732 | 15.525 | 1.00 | 40.33 | A |
| 3236 | O | ALA | A | 1422 | −14.949 | −14.469 | 15.200 | 1.00 | 41.87 | A |
| 3237 | N | TYR | A | 1423 | −12.995 | −13.801 | 15.855 | 1.00 | 35.32 | A |
| 3238 | CA | TYR | A | 1423 | −13.331 | −12.422 | 15.981 | 1.00 | 39.01 | A |
| 3239 | CB | TYR | A | 1423 | −13.610 | −11.833 | 17.469 | 1.00 | 39.45 | A |
| 3240 | CG | TYR | A | 1423 | −14.991 | −12.217 | 17.977 | 1.00 | 41.46 | A |
| 3241 | CD1 | TYR | A | 1423 | −15.219 | −13.483 | 18.665 | 1.00 | 38.54 | A |
| 3242 | CE1 | TYR | A | 1423 | −16.448 | −13.870 | 19.055 | 1.00 | 28.82 | A |
| 3243 | CD2 | TYR | A | 1423 | −16.119 | −11.491 | 17.562 | 1.00 | 37.92 | A |
| 3244 | CE2 | TYR | A | 1423 | −17.404 | −11.962 | 17.897 | 1.00 | 39.03 | A |
| 3245 | CZ | TYR | A | 1423 | −17.572 | −13.143 | 18.669 | 1.00 | 37.94 | A |
| 3246 | OH | TYR | A | 1423 | −18.942 | −13.554 | 19.019 | 1.00 | 40.40 | A |
| 3247 | C | TYR | A | 1423 | −12.028 | −11.926 | 15.566 | 1.00 | 41.82 | A |
| 3248 | O | TYR | A | 1423 | −11.009 | −12.574 | 15.795 | 1.00 | 40.25 | A |
| 3249 | N | ASN | A | 1424 | −12.057 | −10.770 | 14.924 | 1.00 | 44.16 | A |
| 3250 | CA | ASN | A | 1424 | −10.854 | −10.047 | 14.459 | 1.00 | 43.83 | A |
| 3251 | CB | ASN | A | 1424 | −10.756 | −10.119 | 12.971 | 1.00 | 43.72 | A |
| 3252 | CG | ASN | A | 1424 | −10.584 | −11.448 | 12.482 | 1.00 | 48.44 | A |
| 3253 | OD1 | ASN | A | 1424 | −11.502 | −12.177 | 12.329 | 1.00 | 44.59 | A |
| 3254 | ND2 | ASN | A | 1424 | −9.339 | −11.766 | 12.124 | 1.00 | 61.24 | A |
| 3255 | C | ASN | A | 1424 | −11.225 | −8.549 | 14.791 | 1.00 | 43.02 | A |
| 3256 | O | ASN | A | 1424 | −12.504 | −8.116 | 14.705 | 1.00 | 39.62 | A |
| 3257 | N | ILE | A | 1425 | −10.203 | −7.780 | 15.125 | 1.00 | 38.73 | A |
| 3258 | CA | ILE | A | 1425 | −10.619 | −6.569 | 15.755 | 1.00 | 40.72 | A |
| 3259 | CB | ILE | A | 1425 | −9.997 | −6.345 | 17.209 | 1.00 | 40.76 | A |
| 3260 | CG2 | ILE | A | 1425 | −10.446 | −5.052 | 17.595 | 1.00 | 40.31 | A |
| 3261 | CG1 | ILE | A | 1425 | −10.571 | −7.368 | 18.237 | 1.00 | 36.71 | A |
| 3262 | CD1 | ILE | A | 1425 | −9.915 | −8.524 | 18.312 | 1.00 | 28.51 | A |
| 3263 | C | ILE | A | 1425 | −9.995 | −5.630 | 14.845 | 1.00 | 40.80 | A |
| 3264 | O | ILE | A | 1425 | −8.990 | −5.964 | 14.476 | 1.00 | 42.68 | A |
| 3265 | N | LYS | A | 1426 | −10.504 | −4.486 | 14.470 | 1.00 | 40.71 | A |
| 3266 | CA | LYS | A | 1426 | −9.836 | −3.860 | 13.386 | 1.00 | 42.64 | A |
| 3267 | CB | LYS | A | 1426 | −10.517 | −4.136 | 12.001 | 1.00 | 42.04 | A |
| 3268 | CG | LYS | A | 1426 | −9.880 | −3.541 | 10.769 | 1.00 | 39.40 | A |
| 3269 | CD | LYS | A | 1426 | −10.773 | −3.661 | 9.550 | 1.00 | 41.88 | A |
| 3270 | CE | LYS | A | 1426 | −9.789 | −4.282 | 8.261 | 1.00 | 53.13 | A |
| 3271 | NZ | LYS | A | 1426 | −10.336 | −4.322 | 6.784 | 1.00 | 40.24 | A |
| 3272 | C | LYS | A | 1426 | −10.042 | −2.527 | 13.940 | 1.00 | 46.68 | A |
| 3273 | O | LYS | A | 1426 | −11.039 | −2.233 | 14.598 | 1.00 | 48.57 | A |
| 3274 | N | ASP | A | 1427 | −9.025 | −1.720 | 13.785 | 1.00 | 49.22 | A |
| 3275 | CA | ASP | A | 1427 | −9.082 | −0.396 | 14.296 | 1.00 | 51.18 | A |
| 3276 | CB | ASP | A | 1427 | −7.685 | −0.024 | 14.728 | 1.00 | 49.73 | A |
| 3277 | CG | ASP | A | 1427 | −6.794 | 0.320 | 13.539 | 1.00 | 55.99 | A |
| 3278 | OD1 | ASP | A | 1427 | −7.293 | 0.255 | 12.304 | 1.00 | 52.50 | A |
| 3279 | OD2 | ASP | A | 1427 | −5.589 | 0.679 | 13.878 | 1.00 | 58.73 | A |
| 3280 | C | ASP | A | 1427 | −9.638 | 0.538 | 13.175 | 1.00 | 50.89 | A |
| 3281 | O | ASP | A | 1427 | −10.440 | 0.103 | 12.325 | 1.00 | 52.56 | A |
| 3282 | N | SER | A | 1428 | −9.071 | 1.723 | 13.109 | 1.00 | 49.65 | A |
| 3283 | CA | SER | A | 1428 | −9.679 | 2.852 | 12.525 | 1.00 | 52.00 | A |
| 3284 | CB | SER | A | 1428 | −9.644 | 3.951 | 13.648 | 1.00 | 52.54 | A |
| 3285 | OG | SER | A | 1428 | −10.292 | 3.312 | 14.856 | 1.00 | 51.14 | A |
| 3286 | C | SER | A | 1428 | −9.073 | 3.182 | 11.125 | 1.00 | 53.34 | A |
| 3287 | O | SER | A | 1428 | −9.794 | 3.506 | 10.213 | 1.00 | 53.35 | A |
| 3288 | N | THR | A | 1429 | −7.737 | 3.056 | 10.967 | 1.00 | 54.86 | A |
| 3289 | CA | THR | A | 1429 | −7.022 | 3.169 | 9.725 | 1.00 | 55.03 | A |
| 3290 | CB | THR | A | 1429 | −5.550 | 3.276 | 10.086 | 1.00 | 56.05 | A |
| 3291 | OG1 | THR | A | 1429 | −4.960 | 1.975 | 10.172 | 1.00 | 65.20 | A |
| 3292 | CG2 | THR | A | 1429 | −5.340 | 3.775 | 11.475 | 1.00 | 53.66 | A |
| 3293 | C | THR | A | 1429 | −7.309 | 1.821 | 8.944 | 1.00 | 55.45 | A |
| 3294 | O | THR | A | 1429 | −7.377 | 1.776 | 7.684 | 1.00 | 55.57 | A |
| 3295 | N | GLY | A | 1430 | −7.445 | 0.721 | 9.706 | 1.00 | 54.68 | A |
| 3296 | CA | GLY | A | 1430 | −8.210 | −0.392 | 9.290 | 1.00 | 52.85 | A |
| 3297 | C | GLY | A | 1430 | −7.187 | −1.495 | 9.348 | 1.00 | 54.24 | A |
| 3298 | O | GLY | A | 1430 | −7.281 | −2.564 | 8.652 | 1.00 | 56.05 | A |
| 3299 | N | LYS | A | 1431 | −6.219 | −1.348 | 10.233 | 1.00 | 53.34 | A |
| 3300 | CA | LYS | A | 1431 | −5.441 | −2.549 | 10.489 | 1.00 | 51.71 | A |
| 3301 | CB | LYS | A | 1431 | −3.944 | −2.342 | 10.510 | 1.00 | 51.45 | A |
| 3302 | CG | LYS | A | 1431 | −3.327 | −1.005 | 9.796 | 1.00 | 57.45 | A |
| 3303 | CD | LYS | A | 1431 | −2.980 | −1.064 | 8.089 | 1.00 | 58.40 | A |
| 3304 | CE | LYS | A | 1431 | −2.460 | 0.355 | 7.487 | 1.00 | 43.62 | A |
| 3305 | NZ | LYS | A | 1431 | −1.498 | 0.863 | 8.618 | 1.00 | 38.84 | A |
| 3306 | C | LYS | A | 1431 | −6.069 | −3.380 | 11.614 | 1.00 | 51.47 | A |
| 3307 | O | LYS | A | 1431 | −7.201 | −3.124 | 11.948 | 1.00 | 53.10 | A |
| 3308 | N | TYR | A | 1432 | −5.425 | −4.459 | 12.062 | 1.00 | 50.18 | A |
| 3309 | CA | TYR | A | 1432 | −6.044 | −5.664 | 12.549 | 1.00 | 48.23 | A |
| 3310 | CB | TYR | A | 1432 | −5.789 | −6.794 | 11.578 | 1.00 | 47.66 | A |
| 3311 | CG | TYR | A | 1432 | −6.997 | −6.994 | 10.651 | 1.00 | 46.37 | A |
| 3312 | CD1 | TYR | A | 1432 | −8.266 | −7.252 | 11.138 | 1.00 | 32.93 | A |
| 3313 | CE1 | TYR | A | 1432 | −9.282 | −7.328 | 10.365 | 1.00 | 38.32 | A |
| 3314 | CD2 | TYR | A | 1432 | −6.851 | −6.897 | 9.287 | 1.00 | 48.07 | A |
| 3315 | CE2 | TYR | A | 1432 | −7.951 | −6.969 | 8.436 | 1.00 | 43.74 | A |
| 3316 | CZ | TYR | A | 1432 | −9.170 | −7.101 | 8.954 | 1.00 | 43.55 | A |
| 3317 | OH | TYR | A | 1432 | −10.230 | −7.152 | 8.023 | 1.00 | 43.02 | A |
| 3318 | C | TYR | A | 1432 | −5.234 | −5.898 | 13.790 | 1.00 | 51.50 | A |
| 3319 | O | TYR | A | 1432 | −4.035 | −5.458 | 13.792 | 1.00 | 53.44 | A |
| 3320 | N | TRP | A | 1433 | −5.804 | −6.499 | 14.877 | 1.00 | 50.76 | A |
| 3321 | CA | TRP | A | 1433 | −4.994 | −6.864 | 16.093 | 1.00 | 48.32 | A |
| 3322 | CB | TRP | A | 1433 | −5.896 | −7.282 | 17.210 | 1.00 | 44.96 | A |
| 3323 | CG | TRP | A | 1433 | −6.383 | −6.126 | 18.055 | 1.00 | 45.32 | A |
| 3324 | CD2 | TRP | A | 1433 | −6.938 | −6.144 | 19.416 | 1.00 | 43.18 | A |
| 3325 | CE2 | TRP | A | 1433 | −7.239 | −4.828 | 19.733 | 1.00 | 42.41 | A |
| 3326 | CE3 | TRP | A | 1433 | −7.149 | −7.150 | 20.401 | 1.00 | 33.86 | A |
| 3327 | CD1 | TRP | A | 1433 | −6.304 | −4.854 | 17.721 | 1.00 | 42.54 | A |
| 3328 | NE1 | TRP | A | 1433 | −6.854 | −4.080 | 18.670 | 1.00 | 40.10 | A |
| 3329 | CZ2 | TRP | A | 1433 | −7.790 | −4.453 | 20.965 | 1.00 | 38.29 | A |
| 3330 | CZ3 | TRP | A | 1433 | −7.782 | −6.822 | 21.446 | 1.00 | 38.33 | A |
| 3331 | CH2 | TRP | A | 1433 | −8.060 | −5.464 | 21.780 | 1.00 | 40.83 | A |
| 3332 | C | TRP | A | 1433 | −4.071 | −8.063 | 15.901 | 1.00 | 50.43 | A |
| 3333 | O | TRP | A | 1433 | −4.605 | −9.174 | 15.729 | 1.00 | 49.13 | A |
| 3334 | N | THR | A | 1434 | −2.723 | −7.908 | 15.990 | 1.00 | 51.71 | A |
| 3335 | CA | THR | A | 1434 | −1.883 | −9.141 | 16.024 | 1.00 | 51.05 | A |
| 3336 | CB | THR | A | 1434 | −0.774 | −9.105 | 15.092 | 1.00 | 50.46 | A |
| 3337 | OG1 | THR | A | 1434 | −0.454 | −7.738 | 14.904 | 1.00 | 48.07 | A |
| 3338 | CG2 | THR | A | 1434 | −1.235 | −9.854 | 13.746 | 1.00 | 47.58 | A |
| 3339 | C | THR | A | 1434 | −1.314 | −9.522 | 17.312 | 1.00 | 52.82 | A |
| 3340 | O | THR | A | 1434 | −1.373 | −8.752 | 18.253 | 1.00 | 54.18 | A |
| 3341 | N | VAL | A | 1435 | −0.719 | −10.723 | 17.374 | 1.00 | 54.08 | A |
| 3342 | CA | VAL | A | 1435 | 0.134 | −11.010 | 18.537 | 1.00 | 54.41 | A |
| 3343 | CB | VAL | A | 1435 | −0.277 | −12.128 | 19.474 | 1.00 | 54.20 | A |
| 3344 | CG1 | VAL | A | 1435 | 0.733 | −13.135 | 19.662 | 1.00 | 48.55 | A |
| 3345 | CG2 | VAL | A | 1435 | −0.645 | −11.540 | 20.824 | 1.00 | 57.15 | A |
| 3346 | C | VAL | A | 1435 | 1.493 | −11.125 | 18.151 | 1.00 | 56.81 | A |
| 3347 | O | VAL | A | 1435 | 1.848 | −11.581 | 17.024 | 1.00 | 58.07 | A |
| 3348 | N | GLY | A | 1436 | 2.288 | −10.585 | 19.067 | 1.00 | 59.25 | A |
| 3349 | CA | GLY | A | 1436 | 3.758 | −10.626 | 18.907 | 1.00 | 59.56 | A |
| 3350 | C | GLY | A | 1436 | 4.490 | −11.886 | 19.374 | 1.00 | 58.27 | A |
| 3351 | O | GLY | A | 1436 | 4.090 | −12.494 | 20.416 | 1.00 | 58.45 | A |
| 3352 | N | SER | A | 1437 | 5.637 | −12.192 | 18.743 | 1.00 | 56.72 | A |
| 3353 | CA | SER | A | 1437 | 6.467 | −13.301 | 19.269 | 1.00 | 55.51 | A |
| 3354 | CB | SER | A | 1437 | 7.664 | −13.435 | 18.450 | 1.00 | 53.21 | A |
| 3355 | OG | SER | A | 1437 | 8.409 | −12.278 | 18.517 | 1.00 | 45.93 | A |
| 3356 | C | SER | A | 1437 | 6.793 | −13.248 | 20.796 | 1.00 | 57.44 | A |
| 3357 | O | SER | A | 1437 | 7.064 | −14.342 | 21.469 | 1.00 | 58.93 | A |
| 3358 | N | ASP | A | 1438 | 6.725 | −12.061 | 21.422 | 1.00 | 57.22 | A |
| 3359 | CA | ASP | A | 1438 | 6.879 | −12.056 | 22.959 | 1.00 | 58.69 | A |
| 3360 | CB | ASP | A | 1438 | 7.329 | −10.745 | 23.309 | 1.00 | 56.82 | A |
| 3361 | CG | ASP | A | 1438 | 6.683 | −9.839 | 22.430 | 1.00 | 57.61 | A |
| 3362 | OD1 | ASP | A | 1438 | 5.537 | −10.191 | 21.991 | 1.00 | 57.05 | A |
| 3363 | OD2 | ASP | A | 1438 | 7.300 | −8.864 | 22.080 | 1.00 | 59.56 | A |
| 3364 | C | ASP | A | 1438 | 5.482 | −12.204 | 23.558 | 1.00 | 59.84 | A |
| 3365 | O | ASP | A | 1438 | 5.349 | −12.212 | 24.789 | 1.00 | 61.61 | A |
| 3366 | N | SER | A | 1439 | 4.468 | −12.313 | 22.681 | 1.00 | 60.20 | A |
| 3367 | CA | SER | A | 1439 | 3.040 | −12.312 | 23.096 | 1.00 | 62.16 | A |
| 3368 | CB | SER | A | 1439 | 2.770 | −13.162 | 24.340 | 1.00 | 62.58 | A |
| 3369 | OG | SER | A | 1439 | 3.255 | −14.504 | 24.307 | 1.00 | 65.35 | A |
| 3370 | C | SER | A | 1439 | 2.440 | −10.910 | 23.383 | 1.00 | 61.86 | A |
| 3371 | O | SER | A | 1439 | 1.275 | −10.770 | 23.716 | 1.00 | 63.05 | A |
| 3372 | N | ALA | A | 1440 | 3.235 | −9.863 | 23.280 | 1.00 | 61.29 | A |
| 3373 | CA | ALA | A | 1440 | 2.668 | −8.503 | 23.241 | 1.00 | 58.54 | A |
| 3374 | CB | ALA | A | 1440 | 3.775 | −7.512 | 23.328 | 1.00 | 56.88 | A |
| 3375 | C | ALA | A | 1440 | 1.916 | −8.364 | 21.916 | 1.00 | 56.32 | A |
| 3376 | O | ALA | A | 1440 | 2.481 | −8.688 | 20.817 | 1.00 | 53.07 | A |
| 3377 | N | VAL | A | 1441 | 0.641 | −7.926 | 22.074 | 1.00 | 55.21 | A |
| 3378 | CA | VAL | A | 1441 | −0.295 | −7.648 | 20.945 | 1.00 | 53.25 | A |
| 3379 | CB | VAL | A | 1441 | −1.734 | −7.595 | 21.372 | 1.00 | 54.21 | A |
| 3380 | CG1 | VAL | A | 1441 | −2.571 | −8.182 | 20.398 | 1.00 | 51.72 | A |
| 3381 | CG2 | VAL | A | 1441 | −1.940 | −8.353 | 22.696 | 1.00 | 56.26 | A |
| 3382 | C | VAL | A | 1441 | 0.122 | −6.328 | 20.350 | 1.00 | 52.19 | A |
| 3383 | O | VAL | A | 1441 | 1.250 | −5.876 | 20.536 | 1.00 | 53.25 | A |
| 3384 | N | THR | A | 1442 | −0.720 | −5.820 | 19.479 | 1.00 | 50.61 | A |
| 3385 | CA | THR | A | 1442 | −0.357 | −4.847 | 18.429 | 1.00 | 48.21 | A |
| 3386 | CB | THR | A | 1442 | 0.987 | −5.030 | 17.848 | 1.00 | 46.18 | A |
| 3387 | OG1 | THR | A | 1442 | 0.915 | −4.510 | 16.528 | 1.00 | 48.96 | A |
| 3388 | CG2 | THR | A | 1442 | 1.426 | −6.401 | 17.805 | 1.00 | 48.07 | A |
| 3389 | C | THR | A | 1442 | −1.305 | −4.652 | 17.277 | 1.00 | 48.20 | A |
| 3390 | O | THR | A | 1442 | −1.713 | −5.537 | 16.595 | 1.00 | 49.92 | A |
| 3391 | N | SER | A | 1443 | −1.578 | −3.426 | 17.011 | 1.00 | 52.16 | A |
| 3392 | CA | SER | A | 1443 | −2.503 | −3.049 | 16.007 | 1.00 | 55.59 | A |
| 3393 | CB | SER | A | 1443 | −3.056 | −1.695 | 16.333 | 1.00 | 56.01 | A |
| 3394 | OG | SER | A | 1443 | −4.484 | −1.701 | 16.466 | 1.00 | 57.45 | A |
| 3395 | C | SER | A | 1443 | −1.821 | −2.931 | 14.700 | 1.00 | 58.69 | A |
| 3396 | O | SER | A | 1443 | −2.496 | −2.759 | 13.728 | 1.00 | 60.44 | A |
| 3397 | N | SER | A | 1444 | −0.490 | −3.053 | 14.669 | 1.00 | 61.51 | A |
| 3398 | CA | SER | A | 1444 | 0.274 | −2.790 | 13.439 | 1.00 | 63.92 | A |
| 3399 | CB | SER | A | 1444 | 1.697 | −2.336 | 13.761 | 1.00 | 63.64 | A |
| 3400 | OG | SER | A | 1444 | 2.325 | −3.070 | 14.858 | 1.00 | 67.87 | A |
| 3401 | C | SER | A | 1444 | 0.270 | −3.974 | 12.540 | 1.00 | 66.24 | A |
| 3402 | O | SER | A | 1444 | 0.491 | −5.143 | 13.051 | 1.00 | 66.86 | A |
| 3403 | N | GLY | A | 1445 | 0.002 | −3.707 | 11.226 | 1.00 | 66.92 | A |
| 3404 | CA | GLY | A | 1445 | −0.096 | −4.814 | 10.113 | 1.00 | 65.00 | A |
| 3405 | C | GLY | A | 1445 | −1.454 | −5.267 | 9.452 | 1.00 | 64.31 | A |
| 3406 | O | GLY | A | 1445 | −2.528 | −4.974 | 9.969 | 1.00 | 62.56 | A |
| 3407 | N | ASP | A | 1446 | −1.392 | −5.975 | 8.308 | 1.00 | 63.66 | A |
| 3408 | CA | ASP | A | 1446 | −2.624 | −6.436 | 7.559 | 1.00 | 63.58 | A |
| 3409 | CB | ASP | A | 1446 | −2.557 | −5.905 | 6.131 | 1.00 | 63.90 | A |
| 3410 | CG | ASP | A | 1446 | −1.407 | −4.913 | 5.939 | 1.00 | 62.61 | A |
| 3411 | OD1 | ASP | A | 1446 | −1.599 | −3.684 | 6.232 | 1.00 | 63.62 | A |
| 3412 | OD2 | ASP | A | 1446 | −0.320 | −5.370 | 5.511 | 1.00 | 55.74 | A |
| 3413 | C | ASP | A | 1446 | −2.995 | −7.981 | 7.530 | 1.00 | 63.07 | A |
| 3414 | O | ASP | A | 1446 | −3.763 | −8.418 | 6.671 | 1.00 | 61.35 | A |
| 3415 | N | THR | A | 1447 | −2.474 | −8.744 | 8.517 | 1.00 | 63.89 | A |
| 3416 | CA | THR | A | 1447 | −2.700 | −10.187 | 8.736 | 1.00 | 64.88 | A |
| 3417 | CB | THR | A | 1447 | −1.431 | −10.926 | 9.494 | 1.00 | 65.58 | A |
| 3418 | OG1 | THR | A | 1447 | −0.130 | −10.392 | 9.067 | 1.00 | 69.94 | A |
| 3419 | CG2 | THR | A | 1447 | −1.400 | −12.455 | 9.321 | 1.00 | 60.83 | A |
| 3420 | C | THR | A | 1447 | −3.905 | −10.365 | 9.670 | 1.00 | 66.10 | A |
| 3421 | O | THR | A | 1447 | −3.642 | −10.367 | 10.897 | 1.00 | 66.05 | A |
| 3422 | N | PRO | A | 1448 | −5.197 | −10.622 | 9.111 | 1.00 | 65.93 | A |
| 3423 | CD | PRO | A | 1448 | −5.680 | −10.785 | 7.706 | 1.00 | 66.30 | A |
| 3424 | CA | PRO | A | 1448 | −6.324 | −10.868 | 10.036 | 1.00 | 64.70 | A |
| 3425 | CB | PRO | A | 1448 | −7.579 | −10.853 | 9.124 | 1.00 | 64.81 | A |
| 3426 | CG | PRO | A | 1448 | −7.133 | −10.379 | 7.744 | 1.00 | 65.42 | A |
| 3427 | C | PRO | A | 1448 | −6.193 | −12.137 | 10.984 | 1.00 | 62.51 | A |
| 3428 | O | PRO | A | 1448 | −6.207 | −13.241 | 10.520 | 1.00 | 60.82 | A |
| 3429 | N | VAL | A | 1449 | −6.125 | −11.873 | 12.324 | 1.00 | 60.10 | A |
| 3430 | CA | VAL | A | 1449 | −5.858 | −12.872 | 13.317 | 1.00 | 54.97 | A |
| 3431 | CB | VAL | A | 1449 | −4.580 | −12.657 | 14.064 | 1.00 | 55.21 | A |
| 3432 | CG1 | VAL | A | 1449 | −3.703 | −11.655 | 13.327 | 1.00 | 52.90 | A |
| 3433 | CG2 | VAL | A | 1449 | −4.818 | −12.429 | 15.625 | 1.00 | 48.82 | A |
| 3434 | C | VAL | A | 1449 | −6.988 | −13.101 | 14.250 | 1.00 | 53.59 | A |
| 3435 | O | VAL | A | 1449 | −7.364 | −12.125 | 14.977 | 1.00 | 54.14 | A |
| 3436 | N | ASP | A | 1450 | −7.463 | −14.381 | 14.239 | 1.00 | 48.44 | A |
| 3437 | CA | ASP | A | 1450 | −8.496 | −14.913 | 15.077 | 1.00 | 46.93 | A |
| 3438 | CB | ASP | A | 1450 | −8.636 | −16.323 | 14.627 | 1.00 | 47.49 | A |
| 3439 | CG | ASP | A | 1450 | −9.103 | −16.478 | 13.131 | 1.00 | 47.44 | A |
| 3440 | OD1 | ASP | A | 1450 | −9.361 | −17.589 | 12.620 | 1.00 | 46.11 | A |
| 3441 | OD2 | ASP | A | 1450 | −9.209 | −15.513 | 12.398 | 1.00 | 54.11 | A |
| 3442 | C | ASP | A | 1450 | −8.359 | −14.861 | 16.717 | 1.00 | 47.92 | A |
| 3443 | O | ASP | A | 1450 | −7.398 | −15.369 | 17.357 | 1.00 | 45.54 | A |
| 3444 | N | PHE | A | 1451 | −9.377 | −14.277 | 17.367 | 1.00 | 48.96 | A |
| 3445 | CA | PHE | A | 1451 | −9.526 | −14.120 | 18.800 | 1.00 | 46.36 | A |
| 3446 | CB | PHE | A | 1451 | −9.532 | −12.661 | 19.140 | 1.00 | 45.53 | A |
| 3447 | CG | PHE | A | 1451 | −8.203 | −12.035 | 19.061 | 1.00 | 46.73 | A |
| 3448 | CD1 | PHE | A | 1451 | −7.686 | −11.614 | 17.855 | 1.00 | 49.62 | A |
| 3449 | CD2 | PHE | A | 1451 | −7.431 | −11.860 | 20.190 | 1.00 | 47.90 | A |
| 3450 | CE1 | PHE | A | 1451 | −6.393 | −10.946 | 17.778 | 1.00 | 47.91 | A |
| 3451 | CE2 | PHE | A | 1451 | −6.112 | −11.213 | 20.136 | 1.00 | 48.89 | A |
| 3452 | CZ | PHE | A | 1451 | −5.623 | −10.737 | 18.928 | 1.00 | 41.41 | A |
| 3453 | C | PHE | A | 1451 | −10.788 | −14.750 | 19.350 | 1.00 | 46.07 | A |
| 3454 | O | PHE | A | 1451 | −11.675 | −15.109 | 18.645 | 1.00 | 47.20 | A |
| 3455 | N | PHE | A | 1452 | −10.865 | −14.909 | 20.664 | 1.00 | 48.03 | A |
| 3456 | CA | PHE | A | 1452 | −11.949 | −15.699 | 21.296 | 1.00 | 47.95 | A |
| 3457 | CB | PHE | A | 1452 | −11.395 | −17.055 | 21.630 | 1.00 | 49.91 | A |
| 3458 | CG | PHE | A | 1452 | −11.043 | −17.834 | 20.440 | 1.00 | 50.45 | A |
| 3459 | CD1 | PHE | A | 1452 | −9.737 | −17.889 | 19.990 | 1.00 | 55.15 | A |
| 3460 | CD2 | PHE | A | 1452 | −11.991 | −18.431 | 19.717 | 1.00 | 50.68 | A |
| 3461 | CE1 | PHE | A | 1452 | −9.372 | −18.634 | 18.807 | 1.00 | 53.49 | A |
| 3462 | CE2 | PHE | A | 1452 | −11.634 | −19.178 | 18.498 | 1.00 | 51.86 | A |
| 3463 | CZ | PHE | A | 1452 | −10.329 | −19.243 | 18.068 | 1.00 | 54.24 | A |
| 3464 | C | PHE | A | 1452 | −12.488 | −15.068 | 22.518 | 1.00 | 48.10 | A |
| 3465 | O | PHE | A | 1452 | −11.790 | −14.839 | 23.488 | 1.00 | 50.76 | A |
| 3466 | N | PHE | A | 1453 | −13.731 | −14.696 | 22.443 | 1.00 | 47.79 | A |
| 3467 | CA | PHE | A | 1453 | −14.347 | −13.977 | 23.503 | 1.00 | 48.41 | A |
| 3468 | CB | PHE | A | 1453 | −15.414 | −13.061 | 22.954 | 1.00 | 45.25 | A |
| 3469 | CG | PHE | A | 1453 | −14.872 | −11.860 | 22.132 | 1.00 | 44.75 | A |
| 3470 | CD1 | PHE | A | 1453 | −13.640 | −11.824 | 21.675 | 1.00 | 34.91 | A |
| 3471 | CD2 | PHE | A | 1453 | −15.714 | −10.771 | 21.798 | 1.00 | 42.03 | A |
| 3472 | CE1 | PHE | A | 1453 | −13.257 | −10.816 | 20.838 | 1.00 | 39.14 | A |
| 3473 | CE2 | PHE | A | 1453 | −15.330 | −9.676 | 21.024 | 1.00 | 38.07 | A |
| 3474 | CZ | PHE | A | 1453 | −14.092 | −9.670 | 20.550 | 1.00 | 40.58 | A |
| 3475 | C | PHE | A | 1453 | −15.002 | −15.044 | 24.387 | 1.00 | 49.21 | A |
| 3476 | O | PHE | A | 1453 | −15.859 | −15.812 | 23.972 | 1.00 | 50.79 | A |
| 3477 | N | GLU | A | 1454 | −14.626 | −15.119 | 25.631 | 1.00 | 49.46 | A |
| 3478 | CA | GLU | A | 1454 | −15.357 | −16.071 | 26.393 | 1.00 | 49.00 | A |
| 3479 | CB | GLU | A | 1454 | −14.331 | −16.941 | 27.180 | 1.00 | 47.68 | A |
| 3480 | CG | GLU | A | 1454 | −13.467 | −17.792 | 26.247 | 1.00 | 51.72 | A |
| 3481 | CD | GLU | A | 1454 | −12.579 | −18.795 | 26.914 | 1.00 | 53.71 | A |
| 3482 | OE1 | GLU | A | 1454 | −12.019 | −19.751 | 26.172 | 1.00 | 55.27 | A |
| 3483 | OE2 | GLU | A | 1454 | −12.491 | −18.572 | 28.134 | 1.00 | 47.57 | A |
| 3484 | C | GLU | A | 1454 | −16.111 | −15.097 | 27.220 | 1.00 | 47.50 | A |
| 3485 | O | GLU | A | 1454 | −15.500 | −14.412 | 27.970 | 1.00 | 45.84 | A |
| 3486 | N | PHE | A | 1455 | −17.406 | −14.974 | 27.076 | 1.00 | 47.03 | A |
| 3487 | CA | PHE | A | 1455 | −18.071 | −14.134 | 28.071 | 1.00 | 49.03 | A |
| 3488 | CB | PHE | A | 1455 | −19.565 | −13.711 | 27.645 | 1.00 | 49.34 | A |
| 3489 | CG | PHE | A | 1455 | −19.586 | −13.155 | 26.319 | 1.00 | 41.03 | A |
| 3490 | CD1 | PHE | A | 1455 | −19.315 | −13.978 | 25.273 | 1.00 | 34.96 | A |
| 3491 | CD2 | PHE | A | 1455 | −19.687 | −11.824 | 26.150 | 1.00 | 41.19 | A |
| 3492 | CE1 | PHE | A | 1455 | −19.278 | −13.546 | 24.074 | 1.00 | 31.99 | A |
| 3493 | CE2 | PHE | A | 1455 | −19.534 | −11.280 | 24.886 | 1.00 | 37.99 | A |
| 3494 | CZ | PHE | A | 1455 | −19.318 | −12.204 | 23.829 | 1.00 | 40.69 | A |
| 3495 | C | PHE | A | 1455 | −18.087 | −14.785 | 29.445 | 1.00 | 49.24 | A |
| 3496 | O | PHE | A | 1455 | −19.020 | −15.600 | 29.734 | 1.00 | 49.10 | A |
| 3497 | N | CYS | A | 1456 | −17.063 | −14.468 | 30.232 | 1.00 | 49.73 | A |
| 3498 | CA | CYS | A | 1456 | −16.827 | −15.035 | 31.594 | 1.00 | 53.02 | A |
| 3499 | CB | CYS | A | 1456 | −15.500 | −14.609 | 31.970 | 1.00 | 53.38 | A |
| 3500 | SG | CYS | A | 1456 | −14.506 | −15.246 | 30.680 | 1.00 | 66.34 | A |
| 3501 | C | CYS | A | 1456 | −17.681 | −14.453 | 32.705 | 1.00 | 53.38 | A |
| 3502 | O | CYS | A | 1456 | −18.091 | −15.208 | 33.582 | 1.00 | 54.77 | A |
| 3503 | N | ASP | A | 1457 | −17.910 | −13.101 | 32.667 | 1.00 | 52.87 | A |
| 3504 | CA | ASP | A | 1457 | −18.918 | −12.357 | 33.465 | 1.00 | 50.10 | A |
| 3505 | CB | ASP | A | 1457 | −18.345 | −11.161 | 34.156 | 1.00 | 50.96 | A |
| 3506 | CG | ASP | A | 1457 | −19.022 | −10.876 | 35.482 | 1.00 | 49.17 | A |
| 3507 | OD1 | ASP | A | 1457 | −20.198 | −11.346 | 35.663 | 1.00 | 51.38 | A |
| 3508 | OD2 | ASP | A | 1457 | −18.377 | −10.173 | 36.318 | 1.00 | 48.04 | A |
| 3509 | C | ASP | A | 1457 | −20.107 | −11.839 | 32.700 | 1.00 | 48.82 | A |
| 3510 | O | ASP | A | 1457 | −20.280 | −12.103 | 31.525 | 1.00 | 50.55 | A |
| 3511 | N | TYR | A | 1458 | −20.904 | −11.053 | 33.406 | 1.00 | 47.83 | A |
| 3512 | CA | TYR | A | 1458 | −22.208 | −10.677 | 33.059 | 1.00 | 46.54 | A |
| 3513 | CB | TYR | A | 1458 | −22.937 | −10.545 | 34.368 | 1.00 | 48.79 | A |
| 3514 | CG | TYR | A | 1458 | −22.764 | −9.203 | 35.055 | 1.00 | 49.61 | A |
| 3515 | CD1 | TYR | A | 1458 | −23.865 | −8.453 | 35.361 | 1.00 | 45.46 | A |
| 3516 | CE1 | TYR | A | 1458 | −23.747 | −7.214 | 35.897 | 1.00 | 55.73 | A |
| 3517 | CD2 | TYR | A | 1458 | −21.475 | −8.687 | 35.348 | 1.00 | 54.47 | A |
| 3518 | CE2 | TYR | A | 1458 | −21.317 | −7.430 | 35.938 | 1.00 | 58.42 | A |
| 3519 | CZ | TYR | A | 1458 | −22.468 | −6.702 | 36.216 | 1.00 | 58.67 | A |
| 3520 | OH | TYR | A | 1458 | −22.403 | −5.413 | 36.773 | 1.00 | 59.14 | A |
| 3521 | C | TYR | A | 1458 | −22.246 | −9.331 | 32.376 | 1.00 | 48.12 | A |
| 3522 | O | TYR | A | 1458 | −23.308 | −8.793 | 32.265 | 1.00 | 51.05 | A |
| 3523 | N | ASN | A | 1459 | −21.097 | −8.699 | 32.125 | 1.00 | 46.93 | A |
| 3524 | CA | ASN | A | 1459 | −20.954 | −7.643 | 31.212 | 1.00 | 45.04 | A |
| 3525 | CB | ASN | A | 1459 | −21.200 | −6.316 | 31.866 | 1.00 | 46.92 | A |
| 3526 | CG | ASN | A | 1459 | −20.332 | −6.142 | 33.145 | 1.00 | 52.29 | A |
| 3527 | OD1 | ASN | A | 1459 | −20.006 | −7.160 | 33.856 | 1.00 | 46.43 | A |
| 3528 | ND2 | ASN | A | 1459 | −19.887 | −4.858 | 33.418 | 1.00 | 46.81 | A |
| 3529 | C | ASN | A | 1459 | −19.507 | −7.565 | 31.002 | 1.00 | 42.68 | A |
| 3530 | O | ASN | A | 1459 | −19.111 | −6.482 | 30.756 | 1.00 | 44.15 | A |
| 3531 | N | LYS | A | 1460 | −18.743 | −8.638 | 30.964 | 1.00 | 40.59 | A |
| 3532 | CA | LYS | A | 1460 | −17.290 | −8.586 | 30.691 | 1.00 | 42.03 | A |
| 3533 | CB | LYS | A | 1460 | −16.479 | −8.473 | 32.092 | 1.00 | 43.56 | A |
| 3534 | CG | LYS | A | 1460 | −16.398 | −7.019 | 32.761 | 1.00 | 39.23 | A |
| 3535 | CD | LYS | A | 1460 | −15.304 | −6.988 | 33.808 | 1.00 | 46.14 | A |
| 3536 | CE | LYS | A | 1460 | −15.490 | −7.982 | 34.917 | 1.00 | 49.48 | A |
| 3537 | NZ | LYS | A | 1460 | −16.531 | −7.489 | 35.811 | 1.00 | 52.09 | A |
| 3538 | C | LYS | A | 1460 | −16.832 | −9.845 | 29.953 | 1.00 | 39.80 | A |
| 3539 | O | LYS | A | 1460 | −17.543 | −10.769 | 30.034 | 1.00 | 41.84 | A |
| 3540 | N | VAL | A | 1461 | −15.677 | −9.953 | 29.309 | 1.00 | 36.19 | A |
| 3541 | CA | VAL | A | 1461 | −15.442 | −11.045 | 28.506 | 1.00 | 34.67 | A |
| 3542 | CB | VAL | A | 1461 | −15.960 | −10.765 | 26.946 | 1.00 | 35.29 | A |
| 3543 | CG1 | VAL | A | 1461 | −14.998 | −10.003 | 26.204 | 1.00 | 28.83 | A |
| 3544 | CG2 | VAL | A | 1461 | −15.872 | −11.968 | 26.023 | 1.00 | 36.48 | A |
| 3545 | C | VAL | A | 1461 | −13.941 | −11.058 | 28.486 | 1.00 | 36.92 | A |
| 3546 | O | VAL | A | 1461 | −13.362 | −10.079 | 28.612 | 1.00 | 39.29 | A |
| 3547 | N | ALA | A | 1462 | −13.280 | −12.177 | 28.233 | 1.00 | 39.30 | A |
| 3548 | CA | ALA | A | 1462 | −11.867 | −12.347 | 28.275 | 1.00 | 37.95 | A |
| 3549 | CB | ALA | A | 1462 | −11.634 | −13.593 | 29.190 | 1.00 | 35.27 | A |
| 3550 | C | ALA | A | 1462 | −11.519 | −12.684 | 26.863 | 1.00 | 38.33 | A |
| 3551 | O | ALA | A | 1462 | −12.300 | −13.367 | 26.140 | 1.00 | 41.32 | A |
| 3552 | N | ILE | A | 1463 | −10.355 | −12.396 | 26.405 | 1.00 | 38.51 | A |
| 3553 | CA | ILE | A | 1463 | −10.143 | −12.665 | 24.985 | 1.00 | 38.50 | A |
| 3554 | CB | ILE | A | 1463 | −9.727 | −11.348 | 24.332 | 1.00 | 37.98 | A |
| 3555 | CG2 | ILE | A | 1463 | −9.238 | −11.503 | 22.908 | 1.00 | 40.85 | A |
| 3556 | CG1 | ILE | A | 1463 | −10.887 | −10.352 | 24.470 | 1.00 | 36.60 | A |
| 3557 | CD1 | ILE | A | 1463 | −10.579 | −8.888 | 24.352 | 1.00 | 32.29 | A |
| 3558 | C | ILE | A | 1463 | −9.029 | −13.661 | 24.866 | 1.00 | 39.59 | A |
| 3559 | O | ILE | A | 1463 | −7.941 | −13.412 | 25.292 | 1.00 | 41.30 | A |
| 3560 | N | LYS | A | 1464 | −9.250 | −14.790 | 24.254 | 1.00 | 40.49 | A |
| 3561 | CA | LYS | A | 1464 | −8.120 | −15.705 | 23.985 | 1.00 | 40.80 | A |
| 3562 | CB | LYS | A | 1464 | −8.618 | −17.126 | 24.294 | 1.00 | 42.62 | A |
| 3563 | CG | LYS | A | 1464 | −7.557 | −17.949 | 25.036 | 1.00 | 39.71 | A |
| 3564 | CD | LYS | A | 1464 | −7.979 | −19.502 | 25.172 | 1.00 | 42.55 | A |
| 3565 | CE | LYS | A | 1464 | −9.511 | −19.702 | 25.673 | 1.00 | 45.04 | A |
| 3566 | NZ | LYS | A | 1464 | −10.052 | −21.032 | 26.098 | 1.00 | 45.24 | A |
| 3567 | C | LYS | A | 1464 | −7.581 | −15.762 | 22.513 | 1.00 | 41.33 | A |
| 3568 | O | LYS | A | 1464 | −8.347 | −15.626 | 21.534 | 1.00 | 40.17 | A |
| 3569 | N | VAL | A | 1465 | −6.327 | −16.191 | 22.369 | 1.00 | 41.36 | A |
| 3570 | CA | VAL | A | 1465 | −5.649 | −16.233 | 21.066 | 1.00 | 40.38 | A |
| 3571 | CB | VAL | A | 1465 | −4.910 | −14.849 | 20.840 | 1.00 | 40.98 | A |
| 3572 | CG1 | VAL | A | 1465 | −4.346 | −14.356 | 22.179 | 1.00 | 39.64 | A |
| 3573 | CG2 | VAL | A | 1465 | −3.883 | −14.815 | 19.860 | 1.00 | 35.47 | A |
| 3574 | C | VAL | A | 1465 | −4.582 | −17.096 | 21.471 | 1.00 | 42.78 | A |
| 3575 | O | VAL | A | 1465 | −3.640 | −16.664 | 22.051 | 1.00 | 41.85 | A |
| 3576 | N | GLY | A | 1466 | −4.696 | −18.370 | 21.157 | 1.00 | 45.04 | A |
| 3577 | CA | GLY | A | 1466 | −3.477 | −19.147 | 21.223 | 1.00 | 47.14 | A |
| 3578 | C | GLY | A | 1466 | −3.121 | −19.781 | 22.526 | 1.00 | 47.56 | A |
| 3579 | O | GLY | A | 1466 | −1.873 | −20.078 | 22.816 | 1.00 | 50.26 | A |
| 3580 | N | GLY | A | 1467 | −4.163 | −20.128 | 23.238 | 1.00 | 46.53 | A |
| 3581 | CA | GLY | A | 1467 | −3.947 | −20.807 | 24.452 | 1.00 | 50.31 | A |
| 3582 | C | GLY | A | 1467 | −4.282 | −19.895 | 25.596 | 1.00 | 53.05 | A |
| 3583 | O | GLY | A | 1467 | −5.000 | −20.332 | 26.641 | 1.00 | 53.89 | A |
| 3584 | N | ARG | A | 1468 | −3.801 | −18.667 | 25.399 | 1.00 | 50.89 | A |
| 3585 | CA | ARG | A | 1468 | −3.748 | −17.791 | 26.478 | 1.00 | 53.32 | A |
| 3586 | CB | ARG | A | 1468 | −2.338 | −17.001 | 26.442 | 1.00 | 55.76 | A |
| 3587 | CG | ARG | A | 1468 | −1.073 | −17.868 | 26.428 | 1.00 | 46.99 | A |
| 3588 | CD | ARG | A | 1468 | −1.202 | −18.538 | 27.746 | 1.00 | 45.45 | A |
| 3589 | NE | ARG | A | 1468 | −0.471 | −19.808 | 27.859 | 1.00 | 47.73 | A |
| 3590 | CZ | ARG | A | 1468 | 0.844 | −19.881 | 27.791 | 1.00 | 47.37 | A |
| 3591 | NH1 | ARG | A | 1468 | 1.563 | −18.760 | 27.569 | 1.00 | 56.48 | A |
| 3592 | NH2 | ARG | A | 1468 | 1.439 | −21.039 | 27.921 | 1.00 | 41.60 | A |
| 3593 | C | ARG | A | 1468 | −4.877 | −16.807 | 26.359 | 1.00 | 53.13 | A |
| 3594 | O | ARG | A | 1468 | −5.372 | −16.637 | 25.202 | 1.00 | 55.87 | A |
| 3595 | N | TYR | A | 1469 | −5.192 | −16.129 | 27.507 | 1.00 | 51.61 | A |
| 3596 | CA | TYR | A | 1469 | −5.894 | −14.814 | 27.587 | 1.00 | 49.46 | A |
| 3597 | CB | TYR | A | 1469 | −6.890 | −14.823 | 28.669 | 1.00 | 50.49 | A |
| 3598 | CG | TYR | A | 1469 | −7.935 | −15.937 | 28.756 | 1.00 | 54.68 | A |
| 3599 | CD1 | TYR | A | 1469 | −9.222 | −15.732 | 28.356 | 1.00 | 55.27 | A |
| 3600 | CE1 | TYR | A | 1469 | −10.160 | −16.771 | 28.442 | 1.00 | 58.47 | A |
| 3601 | CD2 | TYR | A | 1469 | −7.623 | −17.186 | 29.380 | 1.00 | 59.28 | A |
| 3602 | CE2 | TYR | A | 1469 | −8.511 | −18.226 | 29.452 | 1.00 | 51.69 | A |
| 3603 | CZ | TYR | A | 1469 | −9.798 | −18.006 | 29.040 | 1.00 | 57.44 | A |
| 3604 | OH | TYR | A | 1469 | −10.747 | −19.021 | 29.189 | 1.00 | 55.30 | A |
| 3605 | C | TYR | A | 1469 | −5.130 | −13.412 | 27.715 | 1.00 | 48.55 | A |
| 3606 | O | TYR | A | 1469 | −4.011 | −13.218 | 28.312 | 1.00 | 48.69 | A |
| 3607 | N | LEU | A | 1470 | −5.726 | −12.445 | 27.056 | 1.00 | 48.34 | A |
| 3608 | CA | LEU | A | 1470 | −5.183 | −11.106 | 26.898 | 1.00 | 49.35 | A |
| 3609 | CB | LEU | A | 1470 | −5.929 | −10.282 | 25.900 | 1.00 | 47.63 | A |
| 3610 | CG | LEU | A | 1470 | −5.577 | −10.360 | 24.457 | 1.00 | 44.93 | A |
| 3611 | CD1 | LEU | A | 1470 | −6.481 | −9.403 | 23.854 | 1.00 | 40.32 | A |
| 3612 | CD2 | LEU | A | 1470 | −4.233 | −9.826 | 24.289 | 1.00 | 39.91 | A |
| 3613 | C | LEU | A | 1470 | −5.396 | −10.517 | 28.255 | 1.00 | 51.55 | A |
| 3614 | O | LEU | A | 1470 | −6.476 | −10.639 | 28.950 | 1.00 | 51.00 | A |
| 3615 | N | LYS | A | 1471 | −4.316 | −9.927 | 28.678 | 1.00 | 52.93 | A |
| 3616 | CA | LYS | A | 1471 | −4.345 | −9.576 | 30.039 | 1.00 | 56.56 | A |
| 3617 | CB | LYS | A | 1471 | −4.077 | −10.716 | 31.004 | 1.00 | 50.53 | A |
| 3618 | CG | LYS | A | 1471 | −2.685 | −10.677 | 31.250 | 1.00 | 54.37 | A |
| 3619 | CD | LYS | A | 1471 | −2.279 | −10.222 | 32.528 | 1.00 | 56.79 | A |
| 3620 | CE | LYS | A | 1471 | −0.764 | −10.652 | 32.806 | 1.00 | 58.62 | A |
| 3621 | NZ | LYS | A | 1471 | −0.637 | −10.969 | 34.322 | 1.00 | 62.92 | A |
| 3622 | C | LYS | A | 1471 | −3.300 | −8.500 | 29.976 | 1.00 | 57.55 | A |
| 3623 | O | LYS | A | 1471 | −2.417 | −8.656 | 29.250 | 1.00 | 59.94 | A |
| 3624 | N | GLY | A | 1472 | −3.525 | −7.375 | 30.611 | 1.00 | 59.13 | A |
| 3625 | CA | GLY | A | 1472 | −2.647 | −6.287 | 30.489 | 1.00 | 63.58 | A |
| 3626 | C | GLY | A | 1472 | −1.777 | −6.061 | 31.721 | 1.00 | 66.30 | A |
| 3627 | O | GLY | A | 1472 | −2.080 | −5.133 | 32.621 | 1.00 | 63.79 | A |
| 3628 | N | ASP | A | 1473 | −0.694 | −6.890 | 31.685 | 1.00 | 67.87 | A |
| 3629 | CA | ASP | A | 1473 | 0.444 | −6.962 | 32.687 | 1.00 | 70.16 | A |
| 3630 | CB | ASP | A | 1473 | 1.527 | −7.767 | 32.051 | 1.00 | 70.63 | A |
| 3631 | CG | ASP | A | 1473 | 1.994 | −7.104 | 30.760 | 1.00 | 72.24 | A |
| 3632 | OD1 | ASP | A | 1473 | 3.260 | −7.040 | 30.518 | 1.00 | 66.62 | A |
| 3633 | OD2 | ASP | A | 1473 | 1.013 | −6.614 | 30.052 | 1.00 | 73.17 | A |
| 3634 | C | ASP | A | 1473 | 1.260 | −5.719 | 32.970 | 1.00 | 71.01 | A |
| 3635 | O | ASP | A | 1473 | 0.804 | −4.580 | 32.937 | 1.00 | 72.04 | A |
| 3636 | N | HIS | A | 1474 | 2.541 | −5.975 | 33.197 | 1.00 | 72.45 | A |
| 3637 | CA | HIS | A | 1474 | 3.414 | −4.899 | 33.559 | 1.00 | 71.88 | A |
| 3638 | CB | HIS | A | 1474 | 4.766 | −5.386 | 33.924 | 1.00 | 72.61 | A |
| 3639 | CG | HIS | A | 1474 | 4.960 | −5.301 | 35.344 | 1.00 | 71.92 | A |
| 3640 | CD2 | HIS | A | 1474 | 6.068 | −5.181 | 36.106 | 1.00 | 71.89 | A |
| 3641 | ND1 | HIS | A | 1474 | 4.061 | −5.349 | 36.232 | 1.00 | 72.22 | A |
| 3642 | CE1 | HIS | A | 1474 | 4.304 | −5.274 | 37.431 | 1.00 | 77.40 | A |
| 3643 | NE2 | HIS | A | 1474 | 5.634 | −5.164 | 37.412 | 1.00 | 78.29 | A |
| 3644 | C | HIS | A | 1474 | 3.553 | −3.868 | 32.476 | 1.00 | 72.23 | A |
| 3645 | O | HIS | A | 1474 | 4.003 | −4.129 | 31.295 | 1.00 | 69.18 | A |
| 3646 | N | ALA | A | 1475 | 3.201 | −2.676 | 32.968 | 1.00 | 70.64 | A |
| 3647 | CA | ALA | A | 1475 | 3.357 | −1.498 | 32.169 | 1.00 | 67.26 | A |
| 3648 | CB | ALA | A | 1475 | 4.583 | −1.680 | 31.170 | 1.00 | 66.74 | A |
| 3649 | C | ALA | A | 1475 | 2.080 | −1.146 | 31.468 | 1.00 | 61.44 | A |
| 3650 | O | ALA | A | 1475 | 2.067 | −0.145 | 30.792 | 1.00 | 61.89 | A |
| 3651 | N | GLY | A | 1476 | 1.024 | −1.873 | 31.734 | 1.00 | 57.05 | A |
| 3652 | CA | GLY | A | 1476 | −0.280 | −1.617 | 31.068 | 1.00 | 53.24 | A |
| 3653 | C | GLY | A | 1476 | −0.250 | −2.238 | 29.621 | 1.00 | 52.28 | A |
| 3654 | O | GLY | A | 1476 | −1.158 | −2.055 | 28.870 | 1.00 | 51.59 | A |
| 3655 | N | VAL | A | 1477 | 0.807 | −2.939 | 29.207 | 1.00 | 49.69 | A |
| 3656 | CA | VAL | A | 1477 | 0.851 | −3.372 | 27.863 | 1.00 | 50.07 | A |
| 3657 | CB | VAL | A | 1477 | 2.154 | −4.253 | 27.513 | 1.00 | 50.89 | A |
| 3658 | CG1 | VAL | A | 1477 | 2.428 | −4.245 | 25.949 | 1.00 | 48.16 | A |
| 3659 | CG2 | VAL | A | 1477 | 3.281 | −3.844 | 28.206 | 1.00 | 45.57 | A |
| 3660 | C | VAL | A | 1477 | −0.280 | −4.403 | 27.731 | 1.00 | 52.19 | A |
| 3661 | O | VAL | A | 1477 | −0.834 | −4.872 | 28.801 | 1.00 | 52.43 | A |
| 3662 | N | LEU | A | 1478 | −0.539 | −4.865 | 26.471 | 1.00 | 49.93 | A |
| 3663 | CA | LEU | A | 1478 | −1.521 | −5.950 | 26.268 | 1.00 | 47.41 | A |
| 3664 | CB | LEU | A | 1478 | −2.612 | −5.618 | 25.200 | 1.00 | 49.24 | A |
| 3665 | CG | LEU | A | 1478 | −3.993 | −6.329 | 25.100 | 1.00 | 45.99 | A |
| 3666 | CD1 | LEU | A | 1478 | −4.778 | −6.100 | 26.432 | 1.00 | 51.57 | A |
| 3667 | CD2 | LEU | A | 1478 | −4.949 | −6.002 | 24.067 | 1.00 | 35.50 | A |
| 3668 | C | LEU | A | 1478 | −0.780 | −7.095 | 25.776 | 1.00 | 48.64 | A |
| 3669 | O | LEU | A | 1478 | −0.334 | −7.073 | 24.604 | 1.00 | 48.71 | A |
| 3670 | N | LYS | A | 1479 | −0.680 | −8.137 | 26.605 | 1.00 | 49.41 | A |
| 3671 | CA | LYS | A | 1479 | −0.106 | −9.470 | 26.112 | 1.00 | 50.56 | A |
| 3672 | CB | LYS | A | 1479 | 1.213 | −9.828 | 26.886 | 1.00 | 52.29 | A |
| 3673 | CG | LYS | A | 1479 | 2.028 | −8.684 | 27.466 | 1.00 | 47.09 | A |
| 3674 | CD | LYS | A | 1479 | 3.250 | −8.582 | 26.567 | 1.00 | 45.62 | A |
| 3675 | CE | LYS | A | 1479 | 4.325 | −7.553 | 26.977 | 1.00 | 46.40 | A |
| 3676 | NZ | LYS | A | 1479 | 5.401 | −8.350 | 26.313 | 1.00 | 49.55 | A |
| 3677 | C | LYS | A | 1479 | −1.069 | −10.668 | 26.199 | 1.00 | 50.41 | A |
| 3678 | O | LYS | A | 1479 | −2.094 | −10.625 | 26.850 | 1.00 | 52.36 | A |
| 3679 | N | ALA | A | 1480 | −0.763 | −11.749 | 25.552 | 1.00 | 50.77 | A |
| 3680 | CA | ALA | A | 1480 | −1.536 | −12.891 | 25.732 | 1.00 | 51.99 | A |
| 3681 | CB | ALA | A | 1480 | −1.696 | −13.688 | 24.401 | 1.00 | 50.68 | A |
| 3682 | C | ALA | A | 1480 | −0.677 | −13.678 | 26.680 | 1.00 | 53.93 | A |
| 3683 | O | ALA | A | 1480 | −0.246 | −14.798 | 26.345 | 1.00 | 53.71 | A |
| 3684 | N | SER | A | 1481 | −0.568 | −13.119 | 27.891 | 1.00 | 56.22 | A |
| 3685 | CA | SER | A | 1481 | 0.114 | −13.678 | 29.119 | 1.00 | 55.21 | A |
| 3686 | CB | SER | A | 1481 | 0.855 | −12.578 | 29.945 | 1.00 | 54.52 | A |
| 3687 | OG | SER | A | 1481 | 0.237 | −11.276 | 30.059 | 1.00 | 50.34 | A |
| 3688 | C | SER | A | 1481 | −0.782 | −14.588 | 30.015 | 1.00 | 56.79 | A |
| 3689 | O | SER | A | 1481 | −0.419 | −15.798 | 30.157 | 1.00 | 56.75 | A |
| 3690 | N | ALA | A | 1482 | −1.929 | −14.069 | 30.572 | 1.00 | 54.70 | A |
| 3691 | CA | ALA | A | 1482 | −2.896 | −14.912 | 31.398 | 1.00 | 52.31 | A |
| 3692 | CB | ALA | A | 1482 | −4.216 | −14.281 | 31.588 | 1.00 | 46.25 | A |
| 3693 | C | ALA | A | 1482 | −3.119 | −16.300 | 30.923 | 1.00 | 53.71 | A |
| 3694 | O | ALA | A | 1482 | −3.456 | −16.574 | 29.692 | 1.00 | 58.22 | A |
| 3695 | N | GLU | A | 1483 | −2.950 | −17.199 | 31.862 | 1.00 | 53.94 | A |
| 3696 | CA | GLU | A | 1483 | −3.239 | −18.622 | 31.714 | 1.00 | 54.54 | A |
| 3697 | CB | GLU | A | 1483 | −2.079 | −19.407 | 32.315 | 1.00 | 53.89 | A |
| 3698 | CG | GLU | A | 1483 | −2.303 | −20.887 | 32.186 | 1.00 | 61.71 | A |
| 3699 | CD | GLU | A | 1483 | −1.338 | −21.728 | 33.008 | 1.00 | 66.24 | A |
| 3700 | OE1 | GLU | A | 1483 | −1.585 | −22.967 | 33.133 | 1.00 | 56.00 | A |
| 3701 | OE2 | GLU | A | 1483 | −0.367 | −21.089 | 33.567 | 1.00 | 73.95 | A |
| 3702 | C | GLU | A | 1483 | −4.584 | −18.899 | 32.427 | 1.00 | 56.07 | A |
| 3703 | O | GLU | A | 1483 | −5.386 | −19.988 | 32.178 | 1.00 | 56.67 | A |
| 3704 | N | THR | A | 1484 | −4.935 | −17.935 | 33.309 | 1.00 | 54.44 | A |
| 3705 | CA | THR | A | 1484 | −6.211 | −18.217 | 33.980 | 1.00 | 53.55 | A |
| 3706 | CB | THR | A | 1484 | −6.123 | −18.798 | 35.483 | 1.00 | 51.53 | A |
| 3707 | OG1 | THR | A | 1484 | −4.793 | −19.213 | 35.749 | 1.00 | 56.52 | A |
| 3708 | CG2 | THR | A | 1484 | −6.841 | −19.965 | 35.606 | 1.00 | 40.29 | A |
| 3709 | C | THR | A | 1484 | −6.855 | −16.919 | 33.901 | 1.00 | 54.12 | A |
| 3710 | O | THR | A | 1484 | −6.126 | −15.905 | 33.856 | 1.00 | 55.50 | A |
| 3711 | N | VAL | A | 1485 | −8.196 | −16.925 | 33.951 | 1.00 | 52.19 | A |
| 3712 | CA | VAL | A | 1485 | −8.897 | −15.657 | 33.864 | 1.00 | 51.32 | A |
| 3713 | CB | VAL | A | 1485 | −10.309 | −15.715 | 32.985 | 1.00 | 48.19 | A |
| 3714 | CG1 | VAL | A | 1485 | −10.849 | −17.149 | 32.971 | 1.00 | 58.29 | A |
| 3715 | CG2 | VAL | A | 1485 | −11.392 | −14.859 | 33.417 | 1.00 | 40.22 | A |
| 3716 | C | VAL | A | 1485 | −8.973 | −14.993 | 35.221 | 1.00 | 51.15 | A |
| 3717 | O | VAL | A | 1485 | −9.655 | −15.479 | 36.110 | 1.00 | 50.77 | A |
| 3718 | N | ASP | A | 1486 | −8.368 | −13.828 | 35.299 | 1.00 | 51.89 | A |
| 3719 | CA | ASP | A | 1486 | −8.727 | −12.866 | 36.358 | 1.00 | 55.97 | A |
| 3720 | CB | ASP | A | 1486 | −7.485 | −12.734 | 37.206 | 1.00 | 55.90 | A |
| 3721 | CG | ASP | A | 1486 | −6.481 | −11.990 | 36.463 | 1.00 | 57.66 | A |
| 3722 | OD1 | ASP | A | 1486 | −6.684 | −10.737 | 36.260 | 1.00 | 62.80 | A |
| 3723 | OD2 | ASP | A | 1486 | −5.616 | −12.676 | 35.952 | 1.00 | 57.63 | A |
| 3724 | C | ASP | A | 1486 | −9.068 | −11.380 | 35.899 | 1.00 | 56.45 | A |
| 3725 | O | ASP | A | 1486 | −8.634 | −10.880 | 34.838 | 1.00 | 58.34 | A |
| 3726 | N | PRO | A | 1487 | −9.754 | −10.656 | 36.741 | 1.00 | 55.84 | A |
| 3727 | CD | PRO | A | 1487 | −10.167 | −11.157 | 38.076 | 1.00 | 57.78 | A |
| 3728 | CA | PRO | A | 1487 | −10.097 | −9.276 | 36.648 | 1.00 | 54.70 | A |
| 3729 | CB | PRO | A | 1487 | −9.522 | −8.736 | 38.023 | 1.00 | 54.51 | A |
| 3730 | CG | PRO | A | 1487 | −9.849 | −9.910 | 39.007 | 1.00 | 55.62 | A |
| 3731 | C | PRO | A | 1487 | −9.427 | −8.468 | 35.661 | 1.00 | 52.50 | A |
| 3732 | O | PRO | A | 1487 | −10.029 | −7.516 | 35.213 | 1.00 | 51.21 | A |
| 3733 | N | ALA | A | 1488 | −8.112 | −8.714 | 35.585 | 1.00 | 50.39 | A |
| 3734 | CA | ALA | A | 1488 | −7.133 | −8.040 | 34.731 | 1.00 | 46.94 | A |
| 3735 | CB | ALA | A | 1488 | −5.836 | −7.981 | 35.536 | 1.00 | 46.05 | A |
| 3736 | C | ALA | A | 1488 | −6.852 | −8.805 | 33.309 | 1.00 | 46.32 | A |
| 3737 | O | ALA | A | 1488 | −6.090 | −8.232 | 32.487 | 1.00 | 44.62 | A |
| 3738 | N | SER | A | 1489 | −7.323 | −10.064 | 33.153 | 1.00 | 41.36 | A |
| 3739 | CA | SER | A | 1489 | −7.608 | −10.666 | 31.965 | 1.00 | 45.98 | A |
| 3740 | CB | SER | A | 1489 | −7.192 | −12.156 | 31.976 | 1.00 | 44.84 | A |
| 3741 | OG | SER | A | 1489 | −7.605 | −12.779 | 33.160 | 1.00 | 51.52 | A |
| 3742 | C | SER | A | 1489 | −9.198 | −10.596 | 31.503 | 1.00 | 47.24 | A |
| 3743 | O | SER | A | 1489 | −9.557 | −11.193 | 30.398 | 1.00 | 47.31 | A |
| 3744 | N | LEU | A | 1490 | −10.062 | −9.863 | 32.264 | 1.00 | 46.48 | A |
| 3745 | CA | LEU | A | 1490 | −11.516 | −9.532 | 31.901 | 1.00 | 46.25 | A |
| 3746 | CB | LEU | A | 1490 | −12.472 | −9.752 | 33.102 | 1.00 | 43.27 | A |
| 3747 | CG | LEU | A | 1490 | −12.692 | −11.133 | 33.654 | 1.00 | 41.52 | A |
| 3748 | CD1 | LEU | A | 1490 | −13.573 | −11.124 | 34.917 | 1.00 | 31.63 | A |
| 3749 | CD2 | LEU | A | 1490 | −13.415 | −11.843 | 32.584 | 1.00 | 44.41 | A |
| 3750 | C | LEU | A | 1490 | −11.738 | −8.057 | 31.505 | 1.00 | 46.55 | A |
| 3751 | O | LEU | A | 1490 | −11.339 | −7.161 | 32.183 | 1.00 | 47.47 | A |
| 3752 | N | TRP | A | 1491 | −12.468 | −7.828 | 30.457 | 1.00 | 45.90 | A |
| 3753 | CA | TRP | A | 1491 | −12.609 | −6.545 | 29.841 | 1.00 | 45.88 | A |
| 3754 | CB | TRP | A | 1491 | −12.065 | −6.635 | 28.385 | 1.00 | 48.02 | A |
| 3755 | CG | TRP | A | 1491 | −10.746 | −7.137 | 28.430 | 1.00 | 45.13 | A |
| 3756 | CD2 | TRP | A | 1491 | −9.658 | −6.424 | 28.819 | 1.00 | 41.87 | A |
| 3757 | CE2 | TRP | A | 1491 | −8.575 | −7.284 | 28.773 | 1.00 | 44.90 | A |
| 3758 | CE3 | TRP | A | 1491 | −9.483 | −5.106 | 29.215 | 1.00 | 50.66 | A |
| 3759 | CD1 | TRP | A | 1491 | −10.342 | −8.366 | 28.148 | 1.00 | 49.06 | A |
| 3760 | NE1 | TRP | A | 1491 | −9.029 | −8.495 | 28.352 | 1.00 | 45.38 | A |
| 3761 | CZ2 | TRP | A | 1491 | −7.321 | −6.916 | 29.148 | 1.00 | 51.31 | A |
| 3762 | CZ3 | TRP | A | 1491 | −8.200 | −4.677 | 29.565 | 1.00 | 54.68 | A |
| 3763 | CH2 | TRP | A | 1491 | −7.125 | −5.590 | 29.525 | 1.00 | 53.89 | A |
| 3764 | C | TRP | A | 1491 | −14.117 | −6.324 | 29.660 | 1.00 | 46.86 | A |
| 3765 | O | TRP | A | 1491 | −14.882 | −7.340 | 29.241 | 1.00 | 43.94 | A |
| 3766 | N | GLU | A | 1492 | −14.523 | −5.042 | 29.844 | 1.00 | 43.23 | A |
| 3767 | CA | GLU | A | 1492 | −15.873 | −4.706 | 29.630 | 1.00 | 44.75 | A |
| 3768 | CB | GLU | A | 1492 | −16.198 | −3.429 | 30.378 | 1.00 | 45.84 | A |
| 3769 | CG | GLU | A | 1492 | −17.488 | −3.412 | 31.019 | 1.00 | 45.11 | A |
| 3770 | CD | GLU | A | 1492 | −17.372 | −2.578 | 32.194 | 1.00 | 48.33 | A |
| 3771 | OE1 | GLU | A | 1492 | −17.510 | −1.343 | 32.104 | 1.00 | 55.20 | A |
| 3772 | OE2 | GLU | A | 1492 | −17.074 | −3.143 | 33.228 | 1.00 | 53.19 | A |
| 3773 | C | GLU | A | 1492 | −16.202 | −4.485 | 28.133 | 1.00 | 42.78 | A |
| 3774 | O | GLU | A | 1492 | −15.454 | −4.004 | 27.480 | 1.00 | 41.09 | A |
| 3775 | N | TYR | A | 1493 | −17.360 | −4.780 | 27.598 | 1.00 | 45.74 | A |
| 3776 | CA | TYR | A | 1493 | −17.406 | −4.724 | 26.056 | 1.00 | 46.80 | A |
| 3777 | CB | TYR | A | 1493 | −17.600 | −6.073 | 25.549 | 1.00 | 45.31 | A |
| 3778 | CG | TYR | A | 1493 | −18.755 | −6.856 | 26.255 | 1.00 | 45.14 | A |
| 3779 | CD1 | TYR | A | 1493 | −18.463 | −7.719 | 27.302 | 1.00 | 36.36 | A |
| 3780 | CE1 | TYR | A | 1493 | −19.376 | −8.522 | 27.879 | 1.00 | 28.95 | A |
| 3781 | CD2 | TYR | A | 1493 | −20.104 | −6.767 | 25.820 | 1.00 | 42.06 | A |
| 3782 | CE2 | TYR | A | 1493 | −21.090 | −7.540 | 26.464 | 1.00 | 38.47 | A |
| 3783 | CZ | TYR | A | 1493 | −20.692 | −8.424 | 27.518 | 1.00 | 38.59 | A |
| 3784 | OH | TYR | A | 1493 | −21.559 | −9.275 | 28.205 | 1.00 | 44.03 | A |
| 3785 | C | TYR | A | 1493 | −18.673 | −4.014 | 25.716 | 1.00 | 48.11 | A |
| 3786 | O | TYR | A | 1493 | −19.306 | −3.479 | 26.625 | 1.00 | 49.39 | A |
| 3787 | OXT | TYR | A | 1493 | −19.131 | −3.914 | 24.653 | 1.00 | 48.37 | A |
| 3788 | C | GLY | B | 1005 | 7.990 | 13.573 | 3.876 | 1.00 | 64.92 | B |
| 3789 | O | GLY | B | 1005 | 9.019 | 14.185 | 4.226 | 1.00 | 64.66 | B |
| 3790 | N | GLY | B | 1005 | 6.531 | 11.508 | 4.740 | 1.00 | 59.79 | B |
| 3791 | CA | GLY | B | 1005 | 7.067 | 12.887 | 4.937 | 1.00 | 63.28 | B |
| 3792 | N | THR | B | 1006 | 7.680 | 13.466 | 2.573 | 1.00 | 66.77 | B |
| 3793 | CA | THR | B | 1006 | 8.537 | 14.129 | 1.530 | 1.00 | 67.45 | B |
| 3794 | CB | THR | B | 1006 | 7.949 | 15.654 | 1.402 | 1.00 | 70.67 | B |
| 3795 | OG1 | THR | B | 1006 | 8.456 | 16.487 | 2.477 | 1.00 | 75.20 | B |
| 3796 | CG2 | THR | B | 1006 | 6.299 | 15.719 | 1.405 | 1.00 | 62.21 | B |
| 3797 | C | THR | B | 1006 | 10.116 | 14.031 | 1.899 | 1.00 | 67.92 | B |
| 3798 | O | THR | B | 1006 | 10.460 | 13.193 | 2.827 | 1.00 | 68.16 | B |
| 3799 | N | ALA | B | 1007 | 11.080 | 14.764 | 1.273 | 1.00 | 65.04 | B |
| 3800 | CA | ALA | B | 1007 | 12.410 | 14.921 | 2.053 | 1.00 | 64.21 | B |
| 3801 | CB | ALA | B | 1007 | 13.685 | 14.922 | 1.129 | 1.00 | 65.26 | B |
| 3802 | C | ALA | B | 1007 | 12.577 | 15.999 | 3.294 | 1.00 | 61.50 | B |
| 3803 | O | ALA | B | 1007 | 13.425 | 16.851 | 3.240 | 1.00 | 57.58 | B |
| 3804 | N | GLU | B | 1008 | 11.823 | 15.794 | 4.403 | 1.00 | 60.94 | B |
| 3805 | CA | GLU | B | 1008 | 11.631 | 16.635 | 5.708 | 1.00 | 60.80 | B |
| 3806 | CB | GLU | B | 1008 | 11.518 | 15.777 | 6.968 | 1.00 | 57.07 | B |
| 3807 | CG | GLU | B | 1008 | 10.534 | 14.692 | 6.915 | 1.00 | 62.92 | B |
| 3808 | CD | GLU | B | 1008 | 11.183 | 13.287 | 6.452 | 1.00 | 73.19 | B |
| 3809 | OE1 | GLU | B | 1008 | 12.432 | 13.383 | 6.079 | 1.00 | 73.72 | B |
| 3810 | OE2 | GLU | B | 1008 | 10.458 | 12.142 | 6.465 | 1.00 | 71.21 | B |
| 3811 | C | GLU | B | 1008 | 12.426 | 17.895 | 6.176 | 1.00 | 58.95 | B |
| 3812 | O | GLU | B | 1008 | 13.339 | 17.711 | 6.926 | 1.00 | 59.58 | B |
| 3813 | N | ALA | B | 1009 | 12.037 | 19.131 | 5.774 | 1.00 | 56.80 | B |
| 3814 | CA | ALA | B | 1009 | 12.356 | 20.364 | 6.521 | 1.00 | 55.01 | B |
| 3815 | CB | ALA | B | 1009 | 11.715 | 21.515 | 5.835 | 1.00 | 55.05 | B |
| 3816 | C | ALA | B | 1009 | 11.980 | 20.367 | 8.082 | 1.00 | 52.63 | B |
| 3817 | O | ALA | B | 1009 | 11.005 | 19.810 | 8.502 | 1.00 | 50.81 | B |
| 3818 | N | VAL | B | 1010 | 12.755 | 21.030 | 8.906 | 1.00 | 50.27 | B |
| 3819 | CA | VAL | B | 1010 | 12.409 | 20.968 | 10.293 | 1.00 | 51.74 | B |
| 3820 | CB | VAL | B | 1010 | 13.563 | 21.178 | 11.142 | 1.00 | 47.57 | B |
| 3821 | CG1 | VAL | B | 1010 | 14.723 | 20.588 | 10.475 | 1.00 | 45.96 | B |
| 3822 | CG2 | VAL | B | 1010 | 13.785 | 22.585 | 11.137 | 1.00 | 51.11 | B |
| 3823 | C | VAL | B | 1010 | 11.228 | 21.901 | 10.757 | 1.00 | 53.13 | B |
| 3824 | O | VAL | B | 1010 | 10.946 | 22.951 | 10.167 | 1.00 | 55.83 | B |
| 3825 | N | GLN | B | 1011 | 10.578 | 21.454 | 11.820 | 1.00 | 52.10 | B |
| 3826 | CA | GLN | B | 1011 | 9.512 | 22.091 | 12.463 | 1.00 | 52.38 | B |
| 3827 | CB | GLN | B | 1011 | 8.441 | 21.115 | 13.075 | 1.00 | 50.75 | B |
| 3828 | CG | GLN | B | 1011 | 7.108 | 21.939 | 13.424 | 1.00 | 56.95 | B |
| 3829 | CD | GLN | B | 1011 | 5.739 | 21.204 | 13.695 | 1.00 | 55.34 | B |
| 3830 | OE1 | GLN | B | 1011 | 5.696 | 20.225 | 14.456 | 1.00 | 59.29 | B |
| 3831 | NE2 | GLN | B | 1011 | 4.638 | 21.735 | 13.131 | 1.00 | 46.76 | B |
| 3832 | C | GLN | B | 1011 | 10.225 | 22.873 | 13.517 | 1.00 | 51.70 | B |
| 3833 | O | GLN | B | 1011 | 10.380 | 22.365 | 14.583 | 1.00 | 45.52 | B |
| 3834 | N | ILE | B | 1012 | 10.666 | 24.101 | 13.113 | 1.00 | 55.60 | B |
| 3835 | CA | ILE | B | 1012 | 11.041 | 25.344 | 13.942 | 1.00 | 58.78 | B |
| 3836 | CB | ILE | B | 1012 | 11.089 | 26.714 | 13.076 | 1.00 | 59.46 | B |
| 3837 | CG2 | ILE | B | 1012 | 11.495 | 27.857 | 13.913 | 1.00 | 55.23 | B |
| 3838 | CG1 | ILE | B | 1012 | 12.221 | 26.644 | 12.059 | 1.00 | 63.36 | B |
| 3839 | CD1 | ILE | B | 1012 | 13.616 | 25.989 | 12.842 | 1.00 | 57.05 | B |
| 3840 | C | ILE | B | 1012 | 10.410 | 25.706 | 15.383 | 1.00 | 60.80 | B |
| 3841 | O | ILE | B | 1012 | 9.276 | 26.234 | 15.459 | 1.00 | 59.41 | B |
| 3842 | N | GLN | B | 1013 | 11.216 | 25.457 | 16.480 | 1.00 | 60.63 | B |
| 3843 | CA | GLN | B | 1013 | 10.897 | 25.794 | 17.837 | 1.00 | 58.97 | B |
| 3844 | CB | GLN | B | 1013 | 11.076 | 24.541 | 18.641 | 1.00 | 60.79 | B |
| 3845 | CG | GLN | B | 1013 | 10.482 | 23.382 | 17.910 | 1.00 | 63.21 | B |
| 3846 | CD | GLN | B | 1013 | 9.005 | 23.621 | 17.667 | 1.00 | 61.11 | B |
| 3847 | OE1 | GLN | B | 1013 | 8.135 | 22.783 | 18.041 | 1.00 | 60.59 | B |
| 3848 | NE2 | GLN | B | 1013 | 8.697 | 24.843 | 17.133 | 1.00 | 58.48 | B |
| 3849 | C | GLN | B | 1013 | 11.795 | 26.783 | 18.473 | 1.00 | 57.78 | B |
| 3850 | O | GLN | B | 1013 | 13.008 | 26.686 | 18.289 | 1.00 | 53.44 | B |
| 3851 | N | PHE | B | 1014 | 11.134 | 27.676 | 19.280 | 1.00 | 58.00 | B |
| 3852 | CA | PHE | B | 1014 | 11.704 | 28.781 | 20.132 | 1.00 | 56.40 | B |
| 3853 | CB | PHE | B | 1014 | 12.379 | 29.860 | 19.286 | 1.00 | 58.20 | B |
| 3854 | CG | PHE | B | 1014 | 11.415 | 30.461 | 18.340 | 1.00 | 62.77 | B |
| 3855 | CD1 | PHE | B | 1014 | 10.451 | 31.331 | 18.797 | 1.00 | 64.60 | B |
| 3856 | CD2 | PHE | B | 1014 | 11.371 | 30.020 | 17.001 | 1.00 | 71.90 | B |
| 3857 | CE1 | PHE | B | 1014 | 9.465 | 31.819 | 17.977 | 1.00 | 69.33 | B |
| 3858 | CE2 | PHE | B | 1014 | 10.393 | 30.469 | 16.128 | 1.00 | 73.37 | B |
| 3859 | CZ | PHE | B | 1014 | 9.428 | 31.417 | 16.611 | 1.00 | 71.96 | B |
| 3860 | C | PHE | B | 1014 | 10.633 | 29.465 | 21.020 | 1.00 | 55.30 | B |
| 3861 | O | PHE | B | 1014 | 9.289 | 29.266 | 20.902 | 1.00 | 55.39 | B |
| 3862 | N | GLY | B | 1015 | 11.191 | 30.313 | 21.905 | 1.00 | 51.23 | B |
| 3863 | CA | GLY | B | 1015 | 10.374 | 31.096 | 22.818 | 1.00 | 47.34 | B |
| 3864 | C | GLY | B | 1015 | 11.161 | 32.368 | 22.730 | 1.00 | 48.46 | B |
| 3865 | O | GLY | B | 1015 | 12.378 | 32.312 | 22.310 | 1.00 | 47.58 | B |
| 3866 | N | LEU | B | 1016 | 10.531 | 33.466 | 23.211 | 1.00 | 47.35 | B |
| 3867 | CA | LEU | B | 1016 | 11.038 | 34.834 | 23.137 | 1.00 | 44.99 | B |
| 3868 | CB | LEU | B | 1016 | 10.109 | 35.691 | 22.246 | 1.00 | 43.83 | B |
| 3869 | CG | LEU | B | 1016 | 9.518 | 35.232 | 20.937 | 1.00 | 42.33 | B |
| 3870 | CD1 | LEU | B | 1016 | 10.557 | 34.532 | 19.845 | 1.00 | 37.13 | B |
| 3871 | CD2 | LEU | B | 1016 | 8.474 | 34.330 | 21.507 | 1.00 | 42.02 | B |
| 3872 | C | LEU | B | 1016 | 10.918 | 35.463 | 24.527 | 1.00 | 46.45 | B |
| 3873 | O | LEU | B | 1016 | 10.062 | 35.136 | 25.320 | 1.00 | 46.80 | B |
| 3874 | N | ILE | B | 1017 | 11.676 | 36.469 | 24.794 | 1.00 | 45.92 | B |
| 3875 | CA | ILE | B | 1017 | 11.656 | 36.867 | 26.094 | 1.00 | 49.57 | B |
| 3876 | CB | ILE | B | 1017 | 12.961 | 36.248 | 26.964 | 1.00 | 50.38 | B |
| 3877 | CG2 | ILE | B | 1017 | 12.693 | 36.119 | 28.424 | 1.00 | 47.09 | B |
| 3878 | CG1 | ILE | B | 1017 | 13.577 | 35.024 | 26.360 | 1.00 | 49.64 | B |
| 3879 | CD1 | ILE | B | 1017 | 14.727 | 34.654 | 27.151 | 1.00 | 53.27 | B |
| 3880 | C | ILE | B | 1017 | 11.854 | 38.365 | 26.156 | 1.00 | 50.10 | B |
| 3881 | O | ILE | B | 1017 | 13.035 | 38.891 | 26.035 | 1.00 | 47.25 | B |
| 3882 | N | ASN | B | 1018 | 10.757 | 39.022 | 26.489 | 1.00 | 50.92 | B |
| 3883 | CA | ASN | B | 1018 | 10.871 | 40.466 | 26.669 | 1.00 | 51.32 | B |
| 3884 | CB | ASN | B | 1018 | 9.491 | 41.109 | 26.965 | 1.00 | 50.40 | B |
| 3885 | CG | ASN | B | 1018 | 8.943 | 40.690 | 28.291 | 1.00 | 46.54 | B |
| 3886 | OD1 | ASN | B | 1018 | 7.746 | 40.516 | 28.453 | 1.00 | 39.77 | B |
| 3887 | ND2 | ASN | B | 1018 | 9.853 | 40.431 | 29.229 | 1.00 | 48.41 | B |
| 3888 | C | ASN | B | 1018 | 11.894 | 40.845 | 27.697 | 1.00 | 51.54 | B |
| 3889 | O | ASN | B | 1018 | 12.676 | 40.074 | 28.062 | 1.00 | 50.58 | B |
| 3890 | N | CYS | B | 1019 | 11.696 | 42.036 | 28.195 | 1.00 | 55.22 | B |
| 3891 | CA | CYS | B | 1019 | 12.456 | 42.890 | 29.064 | 1.00 | 57.70 | B |
| 3892 | CB | CYS | B | 1019 | 11.567 | 44.165 | 29.043 | 1.00 | 56.55 | B |
| 3893 | SG | CYS | B | 1019 | 12.001 | 45.371 | 27.807 | 1.00 | 65.01 | B |
| 3894 | C | CYS | B | 1019 | 12.295 | 42.439 | 30.502 | 1.00 | 58.80 | B |
| 3895 | O | CYS | B | 1019 | 13.115 | 42.703 | 31.361 | 1.00 | 60.49 | B |
| 3896 | N | GLY | B | 1020 | 11.122 | 41.955 | 30.837 | 1.00 | 58.81 | B |
| 3897 | CA | GLY | B | 1020 | 10.831 | 41.653 | 32.208 | 1.00 | 58.81 | B |
| 3898 | C | GLY | B | 1020 | 11.404 | 40.259 | 32.355 | 1.00 | 59.74 | B |
| 3899 | O | GLY | B | 1020 | 11.145 | 39.596 | 33.343 | 1.00 | 60.95 | B |
| 3900 | N | ASN | B | 1021 | 12.207 | 39.859 | 31.358 | 1.00 | 57.73 | B |
| 3901 | CA | ASN | B | 1021 | 12.808 | 38.571 | 31.242 | 1.00 | 56.47 | B |
| 3902 | CB | ASN | B | 1021 | 13.832 | 38.263 | 32.256 | 1.00 | 54.86 | B |
| 3903 | CG | ASN | B | 1021 | 15.193 | 38.718 | 31.805 | 1.00 | 59.85 | B |
| 3904 | OD1 | ASN | B | 1021 | 16.038 | 39.103 | 32.602 | 1.00 | 60.36 | B |
| 3905 | ND2 | ASN | B | 1021 | 15.423 | 38.713 | 30.487 | 1.00 | 66.68 | B |
| 3906 | C | ASN | B | 1021 | 11.881 | 37.526 | 31.252 | 1.00 | 57.35 | B |
| 3907 | O | ASN | B | 1021 | 12.305 | 36.464 | 31.428 | 1.00 | 62.51 | B |
| 3908 | N | LYS | B | 1022 | 10.625 | 37.759 | 31.013 | 1.00 | 57.77 | B |
| 3909 | CA | LYS | B | 1022 | 9.603 | 36.766 | 31.149 | 1.00 | 57.49 | B |
| 3910 | CB | LYS | B | 1022 | 8.459 | 37.546 | 31.651 | 1.00 | 59.48 | B |
| 3911 | CG | LYS | B | 1022 | 7.950 | 37.060 | 32.943 | 1.00 | 60.86 | B |
| 3912 | CD | LYS | B | 1022 | 8.944 | 37.431 | 34.053 | 1.00 | 59.38 | B |
| 3913 | CE | LYS | B | 1022 | 8.736 | 38.851 | 34.631 | 1.00 | 47.42 | B |
| 3914 | NZ | LYS | B | 1022 | 7.271 | 38.818 | 35.030 | 1.00 | 42.36 | B |
| 3915 | C | LYS | B | 1022 | 9.249 | 36.287 | 29.749 | 1.00 | 58.01 | B |
| 3916 | O | LYS | B | 1022 | 9.870 | 36.825 | 28.806 | 1.00 | 58.90 | B |
| 3917 | N | TYR | B | 1023 | 8.272 | 35.361 | 29.603 | 1.00 | 56.71 | B |
| 3918 | CA | TYR | B | 1023 | 8.055 | 34.478 | 28.372 | 1.00 | 55.35 | B |
| 3919 | CB | TYR | B | 1023 | 8.273 | 32.988 | 28.655 | 1.00 | 55.47 | B |
| 3920 | CG | TYR | B | 1023 | 9.700 | 32.544 | 28.519 | 1.00 | 54.08 | B |
| 3921 | CD1 | TYR | B | 1023 | 10.519 | 32.491 | 29.614 | 1.00 | 54.83 | B |
| 3922 | CE1 | TYR | B | 1023 | 11.869 | 32.175 | 29.513 | 1.00 | 53.53 | B |
| 3923 | CD2 | TYR | B | 1023 | 10.203 | 32.177 | 27.322 | 1.00 | 48.63 | B |
| 3924 | CE2 | TYR | B | 1023 | 11.535 | 31.900 | 27.180 | 1.00 | 52.63 | B |
| 3925 | CZ | TYR | B | 1023 | 12.395 | 31.916 | 28.303 | 1.00 | 52.61 | B |
| 3926 | OH | TYR | B | 1023 | 13.766 | 31.593 | 28.229 | 1.00 | 51.96 | B |
| 3927 | C | TYR | B | 1023 | 6.697 | 34.426 | 27.722 | 1.00 | 55.90 | B |
| 3928 | O | TYR | B | 1023 | 5.653 | 34.016 | 28.338 | 1.00 | 54.90 | B |
| 3929 | N | LEU | B | 1024 | 6.715 | 34.733 | 26.436 | 1.00 | 56.47 | B |
| 3930 | CA | LEU | B | 1024 | 5.528 | 34.589 | 25.635 | 1.00 | 57.39 | B |
| 3931 | CB | LEU | B | 1024 | 5.984 | 34.633 | 24.273 | 1.00 | 54.25 | B |
| 3932 | CG | LEU | B | 1024 | 5.283 | 35.735 | 23.588 | 1.00 | 57.27 | B |
| 3933 | CD1 | LEU | B | 1024 | 3.482 | 35.743 | 23.281 | 1.00 | 46.48 | B |
| 3934 | CD2 | LEU | B | 1024 | 5.856 | 36.854 | 24.488 | 1.00 | 53.35 | B |
| 3935 | C | LEU | B | 1024 | 4.781 | 33.257 | 25.824 | 1.00 | 60.67 | B |
| 3936 | O | LEU | B | 1024 | 5.323 | 32.152 | 25.452 | 1.00 | 61.55 | B |
| 3937 | N | THR | B | 1025 | 3.552 | 33.317 | 26.384 | 1.00 | 63.02 | B |
| 3938 | CA | THR | B | 1025 | 2.949 | 32.094 | 26.942 | 1.00 | 65.23 | B |
| 3939 | CB | THR | B | 1025 | 3.242 | 31.904 | 28.567 | 1.00 | 65.80 | B |
| 3940 | OG1 | THR | B | 1025 | 4.625 | 31.585 | 28.883 | 1.00 | 65.20 | B |
| 3941 | CG2 | THR | B | 1025 | 2.450 | 30.784 | 29.169 | 1.00 | 64.50 | B |
| 3942 | C | THR | B | 1025 | 1.465 | 31.958 | 26.648 | 1.00 | 66.64 | B |
| 3943 | O | THR | B | 1025 | 0.712 | 32.817 | 26.965 | 1.00 | 68.11 | B |
| 3944 | N | ALA | B | 1026 | 1.043 | 30.834 | 26.095 | 1.00 | 69.67 | B |
| 3945 | CA | ALA | B | 1026 | −0.357 | 30.628 | 25.627 | 1.00 | 72.14 | B |
| 3946 | CB | ALA | B | 1026 | −0.432 | 29.672 | 24.299 | 1.00 | 71.38 | B |
| 3947 | C | ALA | B | 1026 | −1.338 | 30.163 | 26.723 | 1.00 | 72.69 | B |
| 3948 | O | ALA | B | 1026 | −2.359 | 29.440 | 26.434 | 1.00 | 73.33 | B |
| 3949 | N | GLU | B | 1027 | −1.082 | 30.697 | 27.928 | 1.00 | 73.46 | B |
| 3950 | CA | GLU | B | 1027 | −1.721 | 30.257 | 29.210 | 1.00 | 73.22 | B |
| 3951 | CB | GLU | B | 1027 | −1.486 | 31.208 | 30.452 | 1.00 | 73.20 | B |
| 3952 | CG | GLU | B | 1027 | −0.080 | 31.191 | 31.229 | 1.00 | 70.78 | B |
| 3953 | CD | GLU | B | 1027 | 0.235 | 29.964 | 32.077 | 1.00 | 69.66 | B |
| 3954 | OE1 | GLU | B | 1027 | 1.414 | 29.905 | 32.611 | 1.00 | 62.43 | B |
| 3955 | OE2 | GLU | B | 1027 | −0.688 | 29.062 | 32.194 | 1.00 | 67.75 | B |
| 3956 | C | GLU | B | 1027 | −3.204 | 29.870 | 29.157 | 1.00 | 73.10 | B |
| 3957 | O | GLU | B | 1027 | −4.063 | 30.521 | 28.496 | 1.00 | 71.46 | B |
| 3958 | N | ALA | B | 1028 | −3.374 | 28.707 | 29.804 | 1.00 | 73.85 | B |
| 3959 | CA | ALA | B | 1028 | −4.545 | 28.156 | 30.514 | 1.00 | 74.31 | B |
| 3960 | CB | ALA | B | 1028 | −4.145 | 27.891 | 32.072 | 1.00 | 73.59 | B |
| 3961 | C | ALA | B | 1028 | −5.918 | 28.868 | 30.417 | 1.00 | 73.94 | B |
| 3962 | O | ALA | B | 1028 | −6.958 | 28.274 | 30.209 | 1.00 | 69.99 | B |
| 3963 | N | PHE | B | 1029 | −5.851 | 30.159 | 30.732 | 1.00 | 77.81 | B |
| 3964 | CA | PHE | B | 1029 | −6.961 | 31.165 | 30.641 | 1.00 | 77.44 | B |
| 3965 | CB | PHE | B | 1029 | −6.667 | 32.367 | 31.633 | 1.00 | 78.64 | B |
| 3966 | CG | PHE | B | 1029 | −6.854 | 31.987 | 33.189 | 1.00 | 79.84 | B |
| 3967 | CD1 | PHE | B | 1029 | −5.746 | 31.533 | 33.984 | 1.00 | 80.64 | B |
| 3968 | CD2 | PHE | B | 1029 | −8.130 | 32.115 | 33.842 | 1.00 | 82.05 | B |
| 3969 | CE1 | PHE | B | 1029 | −5.876 | 31.163 | 35.382 | 1.00 | 80.19 | B |
| 3970 | CE2 | PHE | B | 1029 | −8.299 | 31.734 | 35.274 | 1.00 | 81.97 | B |
| 3971 | CZ | PHE | B | 1029 | −7.155 | 31.248 | 36.032 | 1.00 | 80.47 | B |
| 3972 | C | PHE | B | 1029 | −7.205 | 31.397 | 29.098 | 1.00 | 75.84 | B |
| 3973 | O | PHE | B | 1029 | −6.275 | 31.839 | 28.322 | 1.00 | 74.53 | B |
| 3974 | N | GLY | B | 1030 | −8.402 | 30.929 | 28.674 | 1.00 | 75.62 | B |
| 3975 | CA | GLY | B | 1030 | −8.652 | 30.462 | 27.280 | 1.00 | 75.55 | B |
| 3976 | C | GLY | B | 1030 | −8.545 | 31.816 | 26.544 | 1.00 | 77.49 | B |
| 3977 | O | GLY | B | 1030 | −9.149 | 32.834 | 27.055 | 1.00 | 79.63 | B |
| 3978 | N | PHE | B | 1031 | −7.786 | 31.897 | 25.428 | 1.00 | 74.40 | B |
| 3979 | CA | PHE | B | 1031 | −7.589 | 33.208 | 24.690 | 1.00 | 72.75 | B |
| 3980 | CB | PHE | B | 1031 | −8.846 | 34.046 | 24.387 | 1.00 | 68.24 | B |
| 3981 | CG | PHE | B | 1031 | −9.837 | 33.363 | 23.486 | 1.00 | 69.64 | B |
| 3982 | CD1 | PHE | B | 1031 | −11.132 | 33.960 | 23.281 | 1.00 | 67.89 | B |
| 3983 | CD2 | PHE | B | 1031 | −9.533 | 32.105 | 22.874 | 1.00 | 61.32 | B |
| 3984 | CE1 | PHE | B | 1031 | −12.104 | 33.316 | 22.457 | 1.00 | 63.21 | B |
| 3985 | CE2 | PHE | B | 1031 | −10.495 | 31.430 | 22.138 | 1.00 | 64.99 | B |
| 3986 | CZ | PHE | B | 1031 | −11.786 | 32.004 | 21.914 | 1.00 | 65.65 | B |
| 3987 | C | PHE | B | 1031 | −6.493 | 34.082 | 25.324 | 1.00 | 72.75 | B |
| 3988 | O | PHE | B | 1031 | −6.683 | 35.264 | 25.541 | 1.00 | 73.78 | B |
| 3989 | N | LYS | B | 1032 | −5.327 | 33.536 | 25.587 | 1.00 | 71.01 | B |
| 3990 | CA | LYS | B | 1032 | −4.342 | 34.402 | 26.136 | 1.00 | 70.20 | B |
| 3991 | CB | LYS | B | 1032 | −3.939 | 34.083 | 27.620 | 1.00 | 70.09 | B |
| 3992 | CG | LYS | B | 1032 | −5.143 | 34.146 | 28.684 | 1.00 | 72.33 | B |
| 3993 | CD | LYS | B | 1032 | −6.079 | 35.438 | 28.598 | 1.00 | 74.05 | B |
| 3994 | CE | LYS | B | 1032 | −7.440 | 35.510 | 29.516 | 1.00 | 68.63 | B |
| 3995 | NZ | LYS | B | 1032 | −7.203 | 35.651 | 30.978 | 1.00 | 60.84 | B |
| 3996 | C | LYS | B | 1032 | −3.301 | 34.050 | 25.215 | 1.00 | 69.02 | B |
| 3997 | O | LYS | B | 1032 | −3.282 | 32.899 | 24.723 | 1.00 | 70.12 | B |
| 3998 | N | VAL | B | 1033 | −2.466 | 35.058 | 24.971 | 1.00 | 66.85 | B |
| 3999 | CA | VAL | B | 1033 | −1.223 | 35.044 | 24.239 | 1.00 | 64.30 | B |
| 4000 | CB | VAL | B | 1033 | −1.343 | 36.107 | 22.996 | 1.00 | 64.56 | B |
| 4001 | CG1 | VAL | B | 1033 | −1.310 | 37.515 | 23.495 | 1.00 | 70.02 | B |
| 4002 | CG2 | VAL | B | 1033 | −0.283 | 36.024 | 21.912 | 1.00 | 59.73 | B |
| 4003 | C | VAL | B | 1033 | −0.284 | 35.545 | 25.295 | 1.00 | 62.81 | B |
| 4004 | O | VAL | B | 1033 | 0.862 | 35.687 | 25.009 | 1.00 | 62.29 | B |
| 4005 | N | ASN | B | 1034 | −0.797 | 35.815 | 26.517 | 1.00 | 61.89 | B |
| 4006 | CA | ASN | B | 1034 | −0.101 | 36.655 | 27.521 | 1.00 | 61.82 | B |
| 4007 | CB | ASN | B | 1034 | −0.537 | 36.492 | 28.959 | 1.00 | 60.74 | B |
| 4008 | CG | ASN | B | 1034 | 0.095 | 35.227 | 29.590 | 1.00 | 64.06 | B |
| 4009 | OD1 | ASN | B | 1034 | 1.324 | 35.197 | 30.012 | 1.00 | 55.00 | B |
| 4010 | ND2 | ASN | B | 1034 | −0.754 | 34.113 | 29.600 | 1.00 | 65.02 | B |
| 4011 | C | ASN | B | 1034 | 1.341 | 36.354 | 27.554 | 1.00 | 62.16 | B |
| 4012 | O | ASN | B | 1034 | 1.695 | 35.224 | 27.080 | 1.00 | 61.69 | B |
| 4013 | N | ALA | B | 1035 | 2.149 | 37.299 | 28.134 | 1.00 | 60.81 | B |
| 4014 | CA | ALA | B | 1035 | 3.551 | 36.953 | 28.387 | 1.00 | 61.41 | B |
| 4015 | CB | ALA | B | 1035 | 4.482 | 37.894 | 27.713 | 1.00 | 62.19 | B |
| 4016 | C | ALA | B | 1035 | 3.878 | 36.785 | 29.881 | 1.00 | 61.65 | B |
| 4017 | O | ALA | B | 1035 | 4.521 | 37.739 | 30.543 | 1.00 | 61.26 | B |
| 4018 | N | SER | B | 1036 | 3.398 | 35.645 | 30.423 | 1.00 | 59.46 | B |
| 4019 | CA | SER | B | 1036 | 3.604 | 35.291 | 31.913 | 1.00 | 61.03 | B |
| 4020 | CB | SER | B | 1036 | 2.340 | 35.158 | 32.752 | 1.00 | 59.54 | B |
| 4021 | OG | SER | B | 1036 | 1.616 | 36.464 | 32.653 | 1.00 | 62.07 | B |
| 4022 | C | SER | B | 1036 | 4.748 | 34.345 | 32.298 | 1.00 | 60.45 | B |
| 4023 | O | SER | B | 1036 | 5.877 | 34.947 | 32.488 | 1.00 | 62.38 | B |
| 4024 | N | ALA | B | 1037 | 4.578 | 32.987 | 32.364 | 1.00 | 56.13 | B |
| 4025 | CA | ALA | B | 1037 | 5.783 | 32.200 | 32.638 | 1.00 | 56.29 | B |
| 4026 | CB | ALA | B | 1037 | 5.711 | 30.953 | 31.927 | 1.00 | 58.17 | B |
| 4027 | C | ALA | B | 1037 | 7.308 | 32.825 | 32.503 | 1.00 | 56.52 | B |
| 4028 | O | ALA | B | 1037 | 7.593 | 33.692 | 31.654 | 1.00 | 56.41 | B |
| 4029 | N | SER | B | 1038 | 8.289 | 32.333 | 33.296 | 1.00 | 54.68 | B |
| 4030 | CA | SER | B | 1038 | 9.589 | 32.970 | 33.360 | 1.00 | 52.82 | B |
| 4031 | CB | SER | B | 1038 | 9.838 | 33.411 | 34.746 | 1.00 | 51.93 | B |
| 4032 | OG | SER | B | 1038 | 9.150 | 32.542 | 35.680 | 1.00 | 57.79 | B |
| 4033 | C | SER | B | 1038 | 10.592 | 31.949 | 33.174 | 1.00 | 52.83 | B |
| 4034 | O | SER | B | 1038 | 11.884 | 32.212 | 33.370 | 1.00 | 54.23 | B |
| 4035 | N | SER | B | 1039 | 10.113 | 30.744 | 32.868 | 1.00 | 51.78 | B |
| 4036 | CA | SER | B | 1039 | 11.044 | 29.648 | 32.548 | 1.00 | 51.19 | B |
| 4037 | CB | SER | B | 1039 | 10.786 | 28.585 | 33.571 | 1.00 | 50.89 | B |
| 4038 | OG | SER | B | 1039 | 11.882 | 28.230 | 34.306 | 1.00 | 52.43 | B |
| 4039 | C | SER | B | 1039 | 10.540 | 29.184 | 31.188 | 1.00 | 52.10 | B |
| 4040 | O | SER | B | 1039 | 9.290 | 29.089 | 31.002 | 1.00 | 53.61 | B |
| 4041 | N | LEU | B | 1040 | 11.391 | 28.834 | 30.225 | 1.00 | 52.66 | B |
| 4042 | CA | LEU | B | 1040 | 10.776 | 28.398 | 28.944 | 1.00 | 53.05 | B |
| 4043 | CB | LEU | B | 1040 | 11.740 | 28.702 | 27.744 | 1.00 | 53.94 | B |
| 4044 | CG | LEU | B | 1040 | 11.555 | 28.372 | 26.190 | 1.00 | 52.12 | B |
| 4045 | CD1 | LEU | B | 1040 | 12.802 | 28.739 | 25.590 | 1.00 | 48.44 | B |
| 4046 | CD2 | LEU | B | 1040 | 11.178 | 26.891 | 25.780 | 1.00 | 39.54 | B |
| 4047 | C | LEU | B | 1040 | 10.393 | 26.944 | 28.899 | 1.00 | 53.66 | B |
| 4048 | O | LEU | B | 1040 | 11.252 | 26.124 | 28.710 | 1.00 | 55.90 | B |
| 4049 | N | LYS | B | 1041 | 9.116 | 26.596 | 28.974 | 1.00 | 55.00 | B |
| 4050 | CA | LYS | B | 1041 | 8.677 | 25.195 | 28.800 | 1.00 | 54.92 | B |
| 4051 | CB | LYS | B | 1041 | 8.561 | 24.460 | 30.115 | 1.00 | 55.47 | B |
| 4052 | CG | LYS | B | 1041 | 9.557 | 24.808 | 31.296 | 1.00 | 57.58 | B |
| 4053 | CD | LYS | B | 1041 | 9.203 | 23.905 | 32.455 | 1.00 | 52.69 | B |
| 4054 | CE | LYS | B | 1041 | 9.808 | 22.419 | 32.321 | 1.00 | 54.24 | B |
| 4055 | NZ | LYS | B | 1041 | 10.878 | 22.068 | 31.294 | 1.00 | 42.28 | B |
| 4056 | C | LYS | B | 1041 | 7.258 | 25.203 | 28.214 | 1.00 | 58.11 | B |
| 4057 | O | LYS | B | 1041 | 6.486 | 26.082 | 28.520 | 1.00 | 59.47 | B |
| 4058 | N | LYS | B | 1042 | 6.870 | 24.164 | 27.456 | 1.00 | 60.17 | B |
| 4059 | CA | LYS | B | 1042 | 5.465 | 23.987 | 27.034 | 1.00 | 59.01 | B |
| 4060 | CB | LYS | B | 1042 | 4.492 | 23.616 | 28.194 | 1.00 | 57.96 | B |
| 4061 | CG | LYS | B | 1042 | 4.342 | 22.099 | 28.660 | 1.00 | 57.77 | B |
| 4062 | CD | LYS | B | 1042 | 5.572 | 21.690 | 29.741 | 1.00 | 62.73 | B |
| 4063 | CE | LYS | B | 1042 | 5.212 | 22.167 | 31.255 | 1.00 | 58.09 | B |
| 4064 | NZ | LYS | B | 1042 | 5.238 | 23.627 | 31.551 | 1.00 | 58.72 | B |
| 4065 | C | LYS | B | 1042 | 4.962 | 25.270 | 26.442 | 1.00 | 59.29 | B |
| 4066 | O | LYS | B | 1042 | 5.409 | 25.779 | 25.370 | 1.00 | 60.85 | B |
| 4067 | N | LYS | B | 1043 | 4.027 | 25.794 | 27.197 | 1.00 | 58.90 | B |
| 4068 | CA | LYS | B | 1043 | 3.087 | 26.850 | 26.778 | 1.00 | 59.81 | B |
| 4069 | CB | LYS | B | 1043 | 2.334 | 27.287 | 28.007 | 1.00 | 58.15 | B |
| 4070 | CG | LYS | B | 1043 | 1.393 | 26.229 | 28.579 | 1.00 | 60.55 | B |
| 4071 | CD | LYS | B | 1043 | 1.025 | 26.629 | 30.093 | 1.00 | 61.33 | B |
| 4072 | CE | LYS | B | 1043 | 2.085 | 26.020 | 31.119 | 1.00 | 61.66 | B |
| 4073 | NZ | LYS | B | 1043 | 3.410 | 25.831 | 30.300 | 1.00 | 48.96 | B |
| 4074 | C | LYS | B | 1043 | 3.729 | 28.064 | 26.010 | 1.00 | 59.15 | B |
| 4075 | O | LYS | B | 1043 | 3.035 | 28.789 | 25.352 | 1.00 | 59.64 | B |
| 4076 | N | GLN | B | 1044 | 5.063 | 28.169 | 26.135 | 1.00 | 60.75 | B |
| 4077 | CA | GLN | B | 1044 | 6.072 | 29.046 | 25.505 | 1.00 | 60.34 | B |
| 4078 | CB | GLN | B | 1044 | 7.128 | 29.288 | 26.510 | 1.00 | 58.68 | B |
| 4079 | CG | GLN | B | 1044 | 6.484 | 30.202 | 27.578 | 1.00 | 64.28 | B |
| 4080 | CD | GLN | B | 1044 | 6.273 | 29.537 | 28.894 | 1.00 | 57.55 | B |
| 4081 | OE1 | GLN | B | 1044 | 7.278 | 29.127 | 29.546 | 1.00 | 56.30 | B |
| 4082 | NE2 | GLN | B | 1044 | 4.980 | 29.329 | 29.234 | 1.00 | 40.71 | B |
| 4083 | C | GLN | B | 1044 | 6.878 | 28.433 | 24.431 | 1.00 | 60.51 | B |
| 4084 | O | GLN | B | 1044 | 7.950 | 28.967 | 24.095 | 1.00 | 59.01 | B |
| 4085 | N | ILE | B | 1045 | 6.459 | 27.293 | 23.883 | 1.00 | 61.21 | B |
| 4086 | CA | ILE | B | 1045 | 7.241 | 26.956 | 22.693 | 1.00 | 60.75 | B |
| 4087 | CB | ILE | B | 1045 | 7.607 | 25.468 | 22.523 | 1.00 | 59.58 | B |
| 4088 | CG2 | ILE | B | 1045 | 9.092 | 25.117 | 23.008 | 1.00 | 54.32 | B |
| 4089 | CG1 | ILE | B | 1045 | 6.477 | 24.595 | 22.973 | 1.00 | 54.86 | B |
| 4090 | CD1 | ILE | B | 1045 | 6.829 | 23.236 | 22.640 | 1.00 | 56.07 | B |
| 4091 | C | ILE | B | 1045 | 6.384 | 27.425 | 21.513 | 1.00 | 63.20 | B |
| 4092 | O | ILE | B | 1045 | 5.125 | 27.133 | 21.460 | 1.00 | 64.50 | B |
| 4093 | N | TRP | B | 1046 | 7.012 | 28.178 | 20.623 | 1.00 | 61.68 | B |
| 4094 | CA | TRP | B | 1046 | 6.201 | 28.684 | 19.570 | 1.00 | 63.72 | B |
| 4095 | CB | TRP | B | 1046 | 6.209 | 30.280 | 19.624 | 1.00 | 66.18 | B |
| 4096 | CG | TRP | B | 1046 | 5.329 | 30.895 | 20.685 | 1.00 | 65.67 | B |
| 4097 | CD2 | TRP | B | 1046 | 3.969 | 31.334 | 20.529 | 1.00 | 70.73 | B |
| 4098 | CE2 | TRP | B | 1046 | 3.551 | 31.828 | 21.812 | 1.00 | 68.49 | B |
| 4099 | CE3 | TRP | B | 1046 | 3.039 | 31.336 | 19.439 | 1.00 | 69.96 | B |
| 4100 | CD1 | TRP | B | 1046 | 5.684 | 31.174 | 21.974 | 1.00 | 65.18 | B |
| 4101 | NE1 | TRP | B | 1046 | 4.628 | 31.695 | 22.656 | 1.00 | 66.91 | B |
| 4102 | CZ2 | TRP | B | 1046 | 2.216 | 32.300 | 22.080 | 1.00 | 65.15 | B |
| 4103 | CZ3 | TRP | B | 1046 | 1.698 | 31.855 | 19.692 | 1.00 | 68.43 | B |
| 4104 | CH2 | TRP | B | 1046 | 1.308 | 32.304 | 21.049 | 1.00 | 66.39 | B |
| 4105 | C | TRP | B | 1046 | 6.879 | 28.151 | 18.315 | 1.00 | 63.04 | B |
| 4106 | O | TRP | B | 1046 | 8.150 | 28.214 | 18.293 | 1.00 | 63.78 | B |
| 4107 | N | THR | B | 1047 | 6.124 | 27.643 | 17.313 | 1.00 | 61.17 | B |
| 4108 | CA | THR | B | 1047 | 6.759 | 27.358 | 15.973 | 1.00 | 62.06 | B |
| 4109 | CB | THR | B | 1047 | 6.550 | 25.960 | 15.513 | 1.00 | 63.12 | B |
| 4110 | OG1 | THR | B | 1047 | 6.693 | 25.112 | 16.677 | 1.00 | 64.91 | B |
| 4111 | CG2 | THR | B | 1047 | 7.542 | 25.485 | 14.189 | 1.00 | 59.96 | B |
| 4112 | C | THR | B | 1047 | 6.517 | 28.288 | 14.784 | 1.00 | 64.10 | B |
| 4113 | O | THR | B | 1047 | 5.526 | 28.135 | 14.057 | 1.00 | 67.36 | B |
| 4114 | N | ALA | B | 1058 | 7.306 | 36.366 | 1.705 | 1.00 | 65.68 | B |
| 4115 | CA | ALA | B | 1058 | 8.233 | 37.129 | 2.494 | 1.00 | 65.53 | B |
| 4116 | CB | ALA | B | 1058 | 8.618 | 38.441 | 1.775 | 1.00 | 65.75 | B |
| 4117 | C | ALA | B | 1058 | 7.692 | 37.434 | 3.917 | 1.00 | 64.37 | B |
| 4118 | O | ALA | B | 1058 | 7.292 | 38.552 | 4.144 | 1.00 | 65.87 | B |
| 4119 | N | ALA | B | 1059 | 7.743 | 36.490 | 4.856 | 1.00 | 61.82 | B |
| 4120 | CA | ALA | B | 1059 | 6.895 | 36.535 | 6.030 | 1.00 | 61.03 | B |
| 4121 | CB | ALA | B | 1059 | 5.581 | 36.911 | 5.579 | 1.00 | 61.18 | B |
| 4122 | C | ALA | B | 1059 | 6.706 | 35.210 | 6.769 | 1.00 | 62.05 | B |
| 4123 | O | ALA | B | 1059 | 6.782 | 34.173 | 6.131 | 1.00 | 61.35 | B |
| 4124 | N | VAL | B | 1060 | 6.280 | 35.245 | 8.063 | 1.00 | 63.12 | B |
| 4125 | CA | VAL | B | 1060 | 5.865 | 34.039 | 8.832 | 1.00 | 60.28 | B |
| 4126 | CB | VAL | B | 1060 | 7.072 | 33.526 | 9.634 | 1.00 | 61.30 | B |
| 4127 | CG1 | VAL | B | 1060 | 8.009 | 32.879 | 8.807 | 1.00 | 54.83 | B |
| 4128 | CG2 | VAL | B | 1060 | 7.719 | 34.581 | 10.358 | 1.00 | 60.86 | B |
| 4129 | C | VAL | B | 1060 | 4.611 | 33.919 | 9.784 | 1.00 | 61.02 | B |
| 4130 | O | VAL | B | 1060 | 4.335 | 34.702 | 10.713 | 1.00 | 62.87 | B |
| 4131 | N | CYS | B | 1061 | 3.924 | 32.810 | 9.682 | 1.00 | 61.05 | B |
| 4132 | CA | CYS | B | 1061 | 3.029 | 32.382 | 10.766 | 1.00 | 62.44 | B |
| 4133 | CB | CYS | B | 1061 | 2.058 | 31.371 | 10.182 | 1.00 | 63.38 | B |
| 4134 | SG | CYS | B | 1061 | 1.385 | 32.179 | 8.752 | 1.00 | 68.62 | B |
| 4135 | C | CYS | B | 1061 | 3.717 | 31.829 | 12.071 | 1.00 | 61.99 | B |
| 4136 | O | CYS | B | 1061 | 4.964 | 31.716 | 12.187 | 1.00 | 61.55 | B |
| 4137 | N | LEU | B | 1062 | 2.914 | 31.508 | 13.065 | 1.00 | 60.64 | B |
| 4138 | CA | LEU | B | 1062 | 3.432 | 31.343 | 14.459 | 1.00 | 59.10 | B |
| 4139 | CB | LEU | B | 1062 | 4.090 | 32.623 | 14.970 | 1.00 | 57.14 | B |
| 4140 | CG | LEU | B | 1062 | 5.621 | 32.632 | 15.273 | 1.00 | 49.68 | B |
| 4141 | CD1 | LEU | B | 1062 | 6.601 | 32.439 | 14.341 | 1.00 | 51.30 | B |
| 4142 | CD2 | LEU | B | 1062 | 5.957 | 33.976 | 15.609 | 1.00 | 48.78 | B |
| 4143 | C | LEU | B | 1062 | 2.250 | 30.818 | 15.341 | 1.00 | 59.81 | B |
| 4144 | O | LEU | B | 1062 | 1.233 | 31.455 | 15.573 | 1.00 | 57.04 | B |
| 4145 | N | ARG | B | 1063 | 2.369 | 29.546 | 15.700 | 1.00 | 61.95 | B |
| 4146 | CA | ARG | B | 1063 | 1.216 | 28.799 | 16.246 | 1.00 | 62.88 | B |
| 4147 | CB | ARG | B | 1063 | 0.933 | 27.526 | 15.343 | 1.00 | 64.18 | B |
| 4148 | CG | ARG | B | 1063 | −0.535 | 26.961 | 15.374 | 1.00 | 63.24 | B |
| 4149 | CD | ARG | B | 1063 | −0.686 | 25.426 | 14.867 | 1.00 | 64.29 | B |
| 4150 | NE | ARG | B | 1063 | 0.415 | 24.501 | 15.185 | 1.00 | 59.18 | B |
| 4151 | CZ | ARG | B | 1063 | 0.203 | 23.410 | 15.952 | 1.00 | 68.64 | B |
| 4152 | NH1 | ARG | B | 1063 | −1.065 | 23.205 | 16.375 | 1.00 | 66.47 | B |
| 4153 | NH2 | ARG | B | 1063 | 1.210 | 22.511 | 16.300 | 1.00 | 67.00 | B |
| 4154 | C | ARG | B | 1063 | 1.434 | 28.482 | 17.742 | 1.00 | 61.65 | B |
| 4155 | O | ARG | B | 1063 | 2.539 | 28.092 | 18.151 | 1.00 | 57.28 | B |
| 4156 | N | SER | B | 1064 | 0.385 | 28.761 | 18.540 | 1.00 | 63.75 | B |
| 4157 | CA | SER | B | 1064 | 0.332 | 28.223 | 19.928 | 1.00 | 67.35 | B |
| 4158 | CB | SER | B | 1064 | −0.939 | 28.671 | 20.736 | 1.00 | 68.21 | B |
| 4159 | OG | SER | B | 1064 | −2.206 | 28.827 | 19.985 | 1.00 | 74.70 | B |
| 4160 | C | SER | B | 1064 | 0.658 | 26.647 | 20.067 | 1.00 | 67.70 | B |
| 4161 | O | SER | B | 1064 | 1.123 | 25.909 | 19.119 | 1.00 | 65.91 | B |
| 4162 | N | HIS | B | 1065 | 0.499 | 26.202 | 21.297 | 1.00 | 67.03 | B |
| 4163 | CA | HIS | B | 1065 | 0.730 | 24.854 | 21.620 | 1.00 | 67.26 | B |
| 4164 | CB | HIS | B | 1065 | 1.100 | 24.685 | 23.099 | 1.00 | 66.20 | B |
| 4165 | CG | HIS | B | 1065 | 1.039 | 23.264 | 23.543 | 1.00 | 68.34 | B |
| 4166 | CD2 | HIS | B | 1065 | 0.032 | 22.557 | 24.128 | 1.00 | 63.90 | B |
| 4167 | ND1 | HIS | B | 1065 | 2.073 | 22.360 | 23.326 | 1.00 | 66.82 | B |
| 4168 | CE1 | HIS | B | 1065 | 1.719 | 21.174 | 23.805 | 1.00 | 62.66 | B |
| 4169 | NE2 | HIS | B | 1065 | 0.471 | 21.252 | 24.253 | 1.00 | 57.22 | B |
| 4170 | C | HIS | B | 1065 | −0.639 | 24.286 | 21.330 | 1.00 | 69.08 | B |
| 4171 | O | HIS | B | 1065 | −0.827 | 23.048 | 21.298 | 1.00 | 70.98 | B |
| 4172 | N | LEU | B | 1066 | −1.613 | 25.171 | 21.071 | 1.00 | 68.60 | B |
| 4173 | CA | LEU | B | 1066 | −2.961 | 24.712 | 21.175 | 1.00 | 67.86 | B |
| 4174 | CB | LEU | B | 1066 | −3.676 | 25.713 | 22.045 | 1.00 | 67.54 | B |
| 4175 | CG | LEU | B | 1066 | −3.111 | 25.389 | 23.481 | 1.00 | 66.72 | B |
| 4176 | CD1 | LEU | B | 1066 | −3.707 | 26.230 | 24.637 | 1.00 | 56.75 | B |
| 4177 | CD2 | LEU | B | 1066 | −3.226 | 23.815 | 23.792 | 1.00 | 64.51 | B |
| 4178 | C | LEU | B | 1066 | −3.737 | 24.261 | 19.875 | 1.00 | 68.67 | B |
| 4179 | O | LEU | B | 1066 | −4.862 | 23.650 | 19.944 | 1.00 | 69.10 | B |
| 4180 | N | GLY | B | 1067 | −3.139 | 24.478 | 18.705 | 1.00 | 66.95 | B |
| 4181 | CA | GLY | B | 1067 | −4.020 | 24.713 | 17.596 | 1.00 | 65.56 | B |
| 4182 | C | GLY | B | 1067 | −3.688 | 25.976 | 16.744 | 1.00 | 65.90 | B |
| 4183 | O | GLY | B | 1067 | −3.534 | 25.845 | 15.481 | 1.00 | 64.00 | B |
| 4184 | N | ARG | B | 1068 | −3.523 | 27.140 | 17.434 | 1.00 | 66.07 | B |
| 4185 | CA | ARG | B | 1068 | −3.941 | 28.538 | 16.965 | 1.00 | 65.03 | B |
| 4186 | CB | ARG | B | 1068 | −4.671 | 29.327 | 18.123 | 1.00 | 68.37 | B |
| 4187 | CG | ARG | B | 1068 | −5.489 | 28.507 | 19.395 | 1.00 | 72.19 | B |
| 4188 | CD | ARG | B | 1068 | −6.736 | 29.219 | 20.333 | 1.00 | 66.71 | B |
| 4189 | NE | ARG | B | 1068 | −6.684 | 28.718 | 21.730 | 1.00 | 72.55 | B |
| 4190 | CZ | ARG | B | 1068 | −6.970 | 29.389 | 22.888 | 1.00 | 77.20 | B |
| 4191 | NH1 | ARG | B | 1068 | −7.437 | 30.676 | 22.890 | 1.00 | 77.35 | B |
| 4192 | NH2 | ARG | B | 1068 | −6.813 | 28.763 | 24.098 | 1.00 | 70.39 | B |
| 4193 | C | ARG | B | 1068 | −2.779 | 29.404 | 16.448 | 1.00 | 62.90 | B |
| 4194 | O | ARG | B | 1068 | −1.614 | 29.164 | 16.793 | 1.00 | 63.68 | B |
| 4195 | N | TYR | B | 1069 | −3.033 | 30.419 | 15.649 | 1.00 | 60.34 | B |
| 4196 | CA | TYR | B | 1069 | −1.881 | 31.417 | 15.247 | 1.00 | 59.14 | B |
| 4197 | CB | TYR | B | 1069 | −1.706 | 31.414 | 13.655 | 1.00 | 57.35 | B |
| 4198 | CG | TYR | B | 1069 | −1.616 | 29.895 | 13.124 | 1.00 | 57.33 | B |
| 4199 | CD1 | TYR | B | 1069 | −0.512 | 29.348 | 12.469 | 1.00 | 56.02 | B |
| 4200 | CE1 | TYR | B | 1069 | −0.497 | 27.960 | 12.040 | 1.00 | 54.72 | B |
| 4201 | CD2 | TYR | B | 1069 | −2.621 | 28.998 | 13.370 | 1.00 | 60.37 | B |
| 4202 | CE2 | TYR | B | 1069 | −2.539 | 27.658 | 12.959 | 1.00 | 57.17 | B |
| 4203 | CZ | TYR | B | 1069 | −1.510 | 27.136 | 12.327 | 1.00 | 53.31 | B |
| 4204 | OH | TYR | B | 1069 | −1.612 | 25.712 | 12.006 | 1.00 | 52.16 | B |
| 4205 | C | TYR | B | 1069 | −1.877 | 32.880 | 16.034 | 1.00 | 58.97 | B |
| 4206 | O | TYR | B | 1069 | −2.986 | 33.361 | 16.552 | 1.00 | 60.02 | B |
| 4207 | N | LEU | B | 1070 | −0.697 | 33.502 | 16.226 | 1.00 | 55.25 | B |
| 4208 | CA | LEU | B | 1070 | −0.585 | 34.885 | 16.670 | 1.00 | 52.60 | B |
| 4209 | CB | LEU | B | 1070 | 0.863 | 35.339 | 16.697 | 1.00 | 49.85 | B |
| 4210 | CG | LEU | B | 1070 | 1.085 | 36.357 | 17.817 | 1.00 | 48.54 | B |
| 4211 | CD1 | LEU | B | 1070 | 0.501 | 35.667 | 19.294 | 1.00 | 48.88 | B |
| 4212 | CD2 | LEU | B | 1070 | 2.458 | 36.843 | 18.016 | 1.00 | 41.09 | B |
| 4213 | C | LEU | B | 1070 | −1.219 | 35.705 | 15.611 | 1.00 | 52.06 | B |
| 4214 | O | LEU | B | 1070 | −1.151 | 35.265 | 14.496 | 1.00 | 52.13 | B |
| 4215 | N | ALA | B | 1071 | −1.759 | 36.898 | 15.947 | 1.00 | 52.27 | B |
| 4216 | CA | ALA | B | 1071 | −2.523 | 37.801 | 15.038 | 1.00 | 51.52 | B |
| 4217 | CB | ALA | B | 1071 | −3.854 | 37.291 | 14.857 | 1.00 | 50.61 | B |
| 4218 | C | ALA | B | 1071 | −2.681 | 39.213 | 15.539 | 1.00 | 51.87 | B |
| 4219 | O | ALA | B | 1071 | −3.630 | 39.507 | 16.261 | 1.00 | 51.87 | B |
| 4220 | N | ALA | B | 1072 | −1.777 | 40.086 | 15.138 | 1.00 | 52.44 | B |
| 4221 | CA | ALA | B | 1072 | −1.904 | 41.565 | 15.468 | 1.00 | 53.12 | B |
| 4222 | CB | ALA | B | 1072 | −0.455 | 42.196 | 15.565 | 1.00 | 50.42 | B |
| 4223 | C | ALA | B | 1072 | −2.777 | 42.418 | 14.441 | 1.00 | 52.44 | B |
| 4224 | O | ALA | B | 1072 | −2.390 | 42.674 | 13.319 | 1.00 | 56.95 | B |
| 4225 | N | ASP | B | 1073 | −3.918 | 42.871 | 14.805 | 1.00 | 49.72 | B |
| 4226 | CA | ASP | B | 1073 | −4.743 | 43.591 | 13.937 | 1.00 | 48.78 | B |
| 4227 | CB | ASP | B | 1073 | −6.137 | 43.521 | 14.517 | 1.00 | 47.79 | B |
| 4228 | CG | ASP | B | 1073 | −6.331 | 44.374 | 15.670 | 1.00 | 43.29 | B |
| 4229 | OD1 | ASP | B | 1073 | −5.534 | 45.213 | 16.012 | 1.00 | 45.27 | B |
| 4230 | OD2 | ASP | B | 1073 | −7.334 | 44.241 | 16.269 | 1.00 | 49.39 | B |
| 4231 | C | ASP | B | 1073 | −4.416 | 45.052 | 13.851 | 1.00 | 51.18 | B |
| 4232 | O | ASP | B | 1073 | −3.441 | 45.537 | 14.481 | 1.00 | 51.00 | B |
| 4233 | N | LYS | B | 1074 | −5.325 | 45.763 | 13.137 | 1.00 | 50.80 | B |
| 4234 | CA | LYS | B | 1074 | −5.168 | 47.177 | 12.895 | 1.00 | 52.35 | B |
| 4235 | CB | LYS | B | 1074 | −6.381 | 47.788 | 12.172 | 1.00 | 51.89 | B |
| 4236 | CG | LYS | B | 1074 | −6.100 | 49.287 | 11.948 | 1.00 | 48.25 | B |
| 4237 | CD | LYS | B | 1074 | −5.875 | 49.636 | 10.434 | 1.00 | 55.99 | B |
| 4238 | CE | LYS | B | 1074 | −4.360 | 49.606 | 9.907 | 1.00 | 55.77 | B |
| 4239 | NZ | LYS | B | 1074 | −4.350 | 49.522 | 8.425 | 1.00 | 52.33 | B |
| 4240 | C | LYS | B | 1074 | −4.880 | 48.101 | 14.085 | 1.00 | 53.30 | B |
| 4241 | O | LYS | B | 1074 | −4.348 | 49.131 | 13.841 | 1.00 | 57.61 | B |
| 4242 | N | ASP | B | 1075 | −5.317 | 47.825 | 15.284 | 1.00 | 53.09 | B |
| 4243 | CA | ASP | B | 1075 | −5.251 | 48.753 | 16.339 | 1.00 | 55.33 | B |
| 4244 | CB | ASP | B | 1075 | −6.635 | 49.012 | 16.957 | 1.00 | 54.01 | B |
| 4245 | CG | ASP | B | 1075 | −7.786 | 49.218 | 15.901 | 1.00 | 54.40 | B |
| 4246 | OD1 | ASP | B | 1075 | −8.724 | 48.411 | 16.022 | 1.00 | 50.97 | B |
| 4247 | OD2 | ASP | B | 1075 | −7.798 | 50.171 | 15.005 | 1.00 | 47.78 | B |
| 4248 | C | ASP | B | 1075 | −4.346 | 48.146 | 17.435 | 1.00 | 58.52 | B |
| 4249 | O | ASP | B | 1075 | −4.369 | 48.624 | 18.656 | 1.00 | 60.90 | B |
| 4250 | N | GLY | B | 1076 | −3.607 | 47.066 | 17.053 | 1.00 | 59.04 | B |
| 4251 | CA | GLY | B | 1076 | −2.483 | 46.443 | 17.865 | 1.00 | 56.50 | B |
| 4252 | C | GLY | B | 1076 | −2.923 | 45.319 | 18.795 | 1.00 | 55.19 | B |
| 4253 | O | GLY | B | 1076 | −2.236 | 44.942 | 19.732 | 1.00 | 53.05 | B |
| 4254 | N | ASN | B | 1077 | −4.081 | 44.805 | 18.470 | 1.00 | 54.96 | B |
| 4255 | CA | ASN | B | 1077 | −4.721 | 43.869 | 19.225 | 1.00 | 57.48 | B |
| 4256 | CB | ASN | B | 1077 | −6.136 | 43.946 | 18.882 | 1.00 | 54.71 | B |
| 4257 | CG | ASN | B | 1077 | −6.914 | 44.969 | 19.758 | 1.00 | 61.91 | B |
| 4258 | OD1 | ASN | B | 1077 | −8.208 | 44.898 | 19.812 | 1.00 | 60.83 | B |
| 4259 | ND2 | ASN | B | 1077 | −6.173 | 45.950 | 20.437 | 1.00 | 57.19 | B |
| 4260 | C | ASN | B | 1077 | −4.133 | 42.413 | 19.061 | 1.00 | 61.30 | B |
| 4261 | O | ASN | B | 1077 | −4.485 | 41.668 | 18.083 | 1.00 | 61.06 | B |
| 4262 | N | VAL | B | 1078 | −3.303 | 41.978 | 20.080 | 1.00 | 62.27 | B |
| 4263 | CA | VAL | B | 1078 | −2.350 | 40.940 | 19.803 | 1.00 | 62.80 | B |
| 4264 | CB | VAL | B | 1078 | −1.069 | 41.283 | 20.344 | 1.00 | 62.28 | B |
| 4265 | CG1 | VAL | B | 1078 | −0.011 | 40.276 | 19.991 | 1.00 | 62.88 | B |
| 4266 | CG2 | VAL | B | 1078 | −0.624 | 42.518 | 19.672 | 1.00 | 64.07 | B |
| 4267 | C | VAL | B | 1078 | −2.851 | 39.635 | 20.270 | 1.00 | 65.30 | B |
| 4268 | O | VAL | B | 1078 | −3.066 | 39.442 | 21.510 | 1.00 | 65.74 | B |
| 4269 | N | THR | B | 1079 | −3.029 | 38.717 | 19.282 | 1.00 | 66.85 | B |
| 4270 | CA | THR | B | 1079 | −3.784 | 37.415 | 19.581 | 1.00 | 69.02 | B |
| 4271 | CB | THR | B | 1079 | −5.225 | 37.333 | 19.058 | 1.00 | 68.35 | B |
| 4272 | OG1 | THR | B | 1079 | −5.587 | 38.601 | 18.552 | 1.00 | 76.10 | B |
| 4273 | CG2 | THR | B | 1079 | −6.132 | 37.002 | 20.142 | 1.00 | 72.01 | B |
| 4274 | C | THR | B | 1079 | −3.157 | 36.146 | 19.053 | 1.00 | 67.90 | B |
| 4275 | O | THR | B | 1079 | −2.138 | 36.206 | 18.358 | 1.00 | 66.97 | B |
| 4276 | N | CYS | B | 1080 | −3.848 | 35.041 | 19.401 | 1.00 | 68.61 | B |
| 4277 | CA | CYS | B | 1080 | −3.474 | 33.639 | 19.181 | 1.00 | 69.78 | B |
| 4278 | CB | CYS | B | 1080 | −2.620 | 33.132 | 20.378 | 1.00 | 69.20 | B |
| 4279 | SG | CYS | B | 1080 | −1.616 | 31.805 | 19.673 | 1.00 | 70.02 | B |
| 4280 | C | CYS | B | 1080 | −4.763 | 32.839 | 19.026 | 1.00 | 70.72 | B |
| 4281 | O | CYS | B | 1080 | −5.008 | 31.879 | 19.751 | 1.00 | 71.53 | B |
| 4282 | N | GLU | B | 1081 | −5.677 | 33.298 | 18.157 | 1.00 | 72.40 | B |
| 4283 | CA | GLU | B | 1081 | −7.017 | 32.678 | 18.125 | 1.00 | 72.00 | B |
| 4284 | CB | GLU | B | 1081 | −8.015 | 33.446 | 18.998 | 1.00 | 73.32 | B |
| 4285 | CG | GLU | B | 1081 | −7.702 | 34.940 | 19.350 | 1.00 | 72.12 | B |
| 4286 | CD | GLU | B | 1081 | −8.810 | 35.626 | 20.295 | 1.00 | 72.68 | B |
| 4287 | OE1 | GLU | B | 1081 | −8.574 | 36.787 | 20.706 | 1.00 | 71.94 | B |
| 4288 | OE2 | GLU | B | 1081 | −9.921 | 35.060 | 20.624 | 1.00 | 76.69 | B |
| 4289 | C | GLU | B | 1081 | −7.597 | 32.316 | 16.746 | 1.00 | 71.26 | B |
| 4290 | O | GLU | B | 1081 | −8.843 | 32.115 | 16.591 | 1.00 | 68.77 | B |
| 4291 | N | ARG | B | 1082 | −6.672 | 32.242 | 15.773 | 1.00 | 71.36 | B |
| 4292 | CA | ARG | B | 1082 | −6.977 | 31.955 | 14.309 | 1.00 | 71.52 | B |
| 4293 | CB | ARG | B | 1082 | −6.777 | 33.121 | 13.294 | 1.00 | 69.86 | B |
| 4294 | CG | ARG | B | 1082 | −5.556 | 34.019 | 13.582 | 1.00 | 73.94 | B |
| 4295 | CD | ARG | B | 1082 | −5.628 | 35.454 | 12.954 | 1.00 | 70.48 | B |
| 4296 | NE | ARG | B | 1082 | −6.857 | 36.193 | 13.343 | 1.00 | 68.79 | B |
| 4297 | CZ | ARG | B | 1082 | −7.401 | 37.153 | 12.588 | 1.00 | 59.85 | B |
| 4298 | NH1 | ARG | B | 1082 | −6.779 | 37.411 | 11.481 | 1.00 | 56.41 | B |
| 4299 | NH2 | ARG | B | 1082 | −8.516 | 37.830 | 12.974 | 1.00 | 53.47 | B |
| 4300 | C | ARG | B | 1082 | −6.217 | 30.754 | 13.883 | 1.00 | 71.79 | B |
| 4301 | O | ARG | B | 1082 | −5.151 | 30.833 | 13.176 | 1.00 | 73.06 | B |
| 4302 | N | GLU | B | 1083 | −6.789 | 29.654 | 14.379 | 1.00 | 71.70 | B |
| 4303 | CA | GLU | B | 1083 | −6.569 | 28.224 | 13.920 | 1.00 | 71.18 | B |
| 4304 | CB | GLU | B | 1083 | −7.813 | 27.372 | 14.255 | 1.00 | 69.91 | B |
| 4305 | CG | GLU | B | 1083 | −8.403 | 27.901 | 15.686 | 1.00 | 70.36 | B |
| 4306 | CD | GLU | B | 1083 | −7.406 | 27.767 | 16.899 | 1.00 | 67.30 | B |
| 4307 | OE1 | GLU | B | 1083 | −7.785 | 28.018 | 18.028 | 1.00 | 65.53 | B |
| 4308 | OE2 | GLU | B | 1083 | −6.260 | 27.304 | 16.736 | 1.00 | 65.12 | B |
| 4309 | C | GLU | B | 1083 | −6.067 | 27.943 | 12.530 | 1.00 | 69.34 | B |
| 4310 | O | GLU | B | 1083 | −5.629 | 26.857 | 12.299 | 1.00 | 70.77 | B |
| 4311 | N | VAL | B | 1084 | −6.048 | 28.981 | 11.720 | 1.00 | 66.88 | B |
| 4312 | CA | VAL | B | 1084 | −6.001 | 28.996 | 10.352 | 1.00 | 66.37 | B |
| 4313 | CB | VAL | B | 1084 | −7.439 | 28.964 | 9.883 | 1.00 | 67.27 | B |
| 4314 | CG1 | VAL | B | 1084 | −8.172 | 27.874 | 10.677 | 1.00 | 68.23 | B |
| 4315 | CG2 | VAL | B | 1084 | −8.142 | 30.333 | 10.118 | 1.00 | 64.32 | B |
| 4316 | C | VAL | B | 1084 | −5.315 | 30.377 | 9.955 | 1.00 | 67.14 | B |
| 4317 | O | VAL | B | 1084 | −5.949 | 31.447 | 9.886 | 1.00 | 66.68 | B |
| 4318 | N | PRO | B | 1085 | −4.007 | 30.323 | 9.645 | 1.00 | 66.33 | B |
| 4319 | CD | PRO | B | 1085 | −3.316 | 29.006 | 9.601 | 1.00 | 63.57 | B |
| 4320 | CA | PRO | B | 1085 | −3.124 | 31.461 | 9.363 | 1.00 | 65.67 | B |
| 4321 | CB | PRO | B | 1085 | −2.071 | 30.816 | 8.527 | 1.00 | 65.31 | B |
| 4322 | CG | PRO | B | 1085 | −2.056 | 29.321 | 9.125 | 1.00 | 64.76 | B |
| 4323 | C | PRO | B | 1085 | −3.605 | 32.713 | 8.633 | 1.00 | 66.26 | B |
| 4324 | O | PRO | B | 1085 | −2.943 | 33.052 | 7.666 | 1.00 | 67.83 | B |
| 4325 | N | GLY | B | 1086 | −4.635 | 33.447 | 9.149 | 1.00 | 68.49 | B |
| 4326 | CA | GLY | B | 1086 | −5.304 | 34.761 | 8.643 | 1.00 | 66.36 | B |
| 4327 | C | GLY | B | 1086 | −4.396 | 35.900 | 8.283 | 1.00 | 68.28 | B |
| 4328 | O | GLY | B | 1086 | −3.214 | 35.669 | 8.027 | 1.00 | 69.71 | B |
| 4329 | N | PRO | B | 1087 | −4.897 | 37.154 | 8.178 | 1.00 | 68.51 | B |
| 4330 | CD | PRO | B | 1087 | −6.181 | 37.763 | 8.611 | 1.00 | 68.98 | B |
| 4331 | CA | PRO | B | 1087 | −3.998 | 38.145 | 7.446 | 1.00 | 67.40 | B |
| 4332 | CB | PRO | B | 1087 | −5.017 | 39.159 | 6.857 | 1.00 | 67.88 | B |
| 4333 | CG | PRO | B | 1087 | −6.396 | 38.972 | 7.682 | 1.00 | 66.71 | B |
| 4334 | C | PRO | B | 1087 | −3.074 | 38.881 | 8.421 | 1.00 | 67.03 | B |
| 4335 | O | PRO | B | 1087 | −1.953 | 39.342 | 8.105 | 1.00 | 63.67 | B |
| 4336 | N | ASP | B | 1088 | −3.652 | 38.974 | 9.629 | 1.00 | 67.76 | B |
| 4337 | CA | ASP | B | 1088 | −3.139 | 39.747 | 10.716 | 1.00 | 67.98 | B |
| 4338 | CB | ASP | B | 1088 | −4.303 | 40.222 | 11.596 | 1.00 | 68.91 | B |
| 4339 | CG | ASP | B | 1088 | −4.957 | 41.565 | 11.063 | 1.00 | 76.04 | B |
| 4340 | OD1 | ASP | B | 1088 | −6.208 | 41.698 | 10.867 | 1.00 | 79.34 | B |
| 4341 | OD2 | ASP | B | 1088 | −4.199 | 42.535 | 10.851 | 1.00 | 79.03 | B |
| 4342 | C | ASP | B | 1088 | −2.202 | 38.838 | 11.443 | 1.00 | 66.00 | B |
| 4343 | O | ASP | B | 1088 | −1.981 | 39.044 | 12.640 | 1.00 | 66.17 | B |
| 4344 | N | CYS | B | 1089 | −1.656 | 37.869 | 10.691 | 1.00 | 64.62 | B |
| 4345 | CA | CYS | B | 1089 | −0.824 | 36.704 | 11.187 | 1.00 | 64.72 | B |
| 4346 | CB | CYS | B | 1089 | −1.287 | 35.358 | 10.684 | 1.00 | 60.77 | B |
| 4347 | SG | CYS | B | 1089 | −2.826 | 34.853 | 11.496 | 1.00 | 63.68 | B |
| 4348 | C | CYS | B | 1089 | 0.665 | 36.742 | 10.897 | 1.00 | 66.26 | B |
| 4349 | O | CYS | B | 1089 | 1.465 | 36.623 | 11.851 | 1.00 | 69.18 | B |
| 4350 | N | ARG | B | 1090 | 1.059 | 36.913 | 9.624 | 1.00 | 65.36 | B |
| 4351 | CA | ARG | B | 1090 | 2.460 | 37.099 | 9.289 | 1.00 | 64.65 | B |
| 4352 | CB | ARG | B | 1090 | 2.565 | 37.640 | 7.826 | 1.00 | 64.12 | B |
| 4353 | CG | ARG | B | 1090 | 1.489 | 37.069 | 6.766 | 1.00 | 67.29 | B |
| 4354 | CD | ARG | B | 1090 | 2.011 | 37.218 | 5.172 | 1.00 | 67.93 | B |
| 4355 | NE | ARG | B | 1090 | 2.430 | 38.579 | 4.633 | 1.00 | 55.08 | B |
| 4356 | CZ | ARG | B | 1090 | 3.162 | 38.658 | 3.563 | 1.00 | 59.33 | B |
| 4357 | NH1 | ARG | B | 1090 | 3.673 | 37.484 | 3.032 | 1.00 | 59.93 | B |
| 4358 | NH2 | ARG | B | 1090 | 3.442 | 39.867 | 3.047 | 1.00 | 55.54 | B |
| 4359 | C | ARG | B | 1090 | 3.244 | 38.009 | 10.320 | 1.00 | 62.38 | B |
| 4360 | O | ARG | B | 1090 | 2.648 | 38.917 | 10.944 | 1.00 | 63.64 | B |
| 4361 | N | PHE | B | 1091 | 4.551 | 37.856 | 10.437 | 1.00 | 59.56 | B |
| 4362 | CA | PHE | B | 1091 | 5.337 | 38.887 | 11.094 | 1.00 | 59.74 | B |
| 4363 | CB | PHE | B | 1091 | 5.604 | 38.618 | 12.671 | 1.00 | 58.40 | B |
| 4364 | CG | PHE | B | 1091 | 4.314 | 38.663 | 13.538 | 1.00 | 55.93 | B |
| 4365 | CD1 | PHE | B | 1091 | 3.823 | 39.918 | 14.019 | 1.00 | 54.95 | B |
| 4366 | CD2 | PHE | B | 1091 | 3.573 | 37.521 | 13.795 | 1.00 | 48.46 | B |
| 4367 | CE1 | PHE | B | 1091 | 2.624 | 40.048 | 14.684 | 1.00 | 45.72 | B |
| 4368 | CE2 | PHE | B | 1091 | 2.387 | 37.621 | 14.481 | 1.00 | 47.05 | B |
| 4369 | CZ | PHE | B | 1091 | 1.911 | 38.917 | 14.930 | 1.00 | 51.42 | B |
| 4370 | C | PHE | B | 1091 | 6.624 | 38.954 | 10.325 | 1.00 | 58.88 | B |
| 4371 | O | PHE | B | 1091 | 7.035 | 37.971 | 9.775 | 1.00 | 58.32 | B |
| 4372 | N | LEU | B | 1092 | 7.237 | 40.121 | 10.332 | 1.00 | 58.29 | B |
| 4373 | CA | LEU | B | 1092 | 8.563 | 40.312 | 9.800 | 1.00 | 58.12 | B |
| 4374 | CB | LEU | B | 1092 | 8.583 | 41.465 | 8.772 | 1.00 | 57.50 | B |
| 4375 | CG | LEU | B | 1092 | 7.239 | 41.467 | 7.980 | 1.00 | 53.91 | B |
| 4376 | CD1 | LEU | B | 1092 | 7.057 | 42.722 | 7.004 | 1.00 | 38.98 | B |
| 4377 | CD2 | LEU | B | 1092 | 7.295 | 40.108 | 7.296 | 1.00 | 39.47 | B |
| 4378 | C | LEU | B | 1092 | 9.644 | 40.482 | 10.927 | 1.00 | 58.88 | B |
| 4379 | O | LEU | B | 1092 | 10.073 | 41.595 | 11.330 | 1.00 | 56.19 | B |
| 4380 | N | ILE | B | 1093 | 10.073 | 39.286 | 11.366 | 1.00 | 58.65 | B |
| 4381 | CA | ILE | B | 1093 | 11.314 | 39.043 | 12.099 | 1.00 | 54.71 | B |
| 4382 | CB | ILE | B | 1093 | 11.723 | 37.540 | 12.129 | 1.00 | 51.84 | B |
| 4383 | CG2 | ILE | B | 1093 | 12.622 | 37.266 | 13.218 | 1.00 | 49.87 | B |
| 4384 | CG1 | ILE | B | 1093 | 10.607 | 36.678 | 12.522 | 1.00 | 50.76 | B |
| 4385 | CD1 | ILE | B | 1093 | 9.480 | 37.266 | 12.147 | 1.00 | 50.85 | B |
| 4386 | C | ILE | B | 1093 | 12.434 | 39.614 | 11.406 | 1.00 | 55.29 | B |
| 4387 | O | ILE | B | 1093 | 13.032 | 38.900 | 10.585 | 1.00 | 58.10 | B |
| 4388 | N | VAL | B | 1094 | 12.861 | 40.816 | 11.725 | 1.00 | 55.30 | B |
| 4389 | CA | VAL | B | 1094 | 14.289 | 40.870 | 11.435 | 1.00 | 57.24 | B |
| 4390 | CB | VAL | B | 1094 | 14.781 | 41.594 | 10.056 | 1.00 | 57.58 | B |
| 4391 | CG1 | VAL | B | 1094 | 16.361 | 41.586 | 9.889 | 1.00 | 59.29 | B |
| 4392 | CG2 | VAL | B | 1094 | 14.045 | 41.086 | 8.730 | 1.00 | 46.01 | B |
| 4393 | C | VAL | B | 1094 | 15.046 | 41.201 | 12.665 | 1.00 | 60.36 | B |
| 4394 | O | VAL | B | 1094 | 14.892 | 42.305 | 13.182 | 1.00 | 64.23 | B |
| 4395 | N | ALA | B | 1095 | 15.836 | 40.220 | 13.143 | 1.00 | 60.88 | B |
| 4396 | CA | ALA | B | 1095 | 16.791 | 40.438 | 14.219 | 1.00 | 61.39 | B |
| 4397 | CB | ALA | B | 1095 | 17.694 | 39.319 | 14.359 | 1.00 | 63.36 | B |
| 4398 | C | ALA | B | 1095 | 17.626 | 41.540 | 14.028 | 1.00 | 61.00 | B |
| 4399 | O | ALA | B | 1095 | 17.699 | 41.997 | 12.960 | 1.00 | 61.06 | B |
| 4400 | N | HIS | B | 1096 | 18.313 | 41.944 | 15.096 | 1.00 | 64.16 | B |
| 4401 | CA | HIS | B | 1096 | 19.461 | 42.876 | 14.966 | 1.00 | 65.11 | B |
| 4402 | CB | HIS | B | 1096 | 19.259 | 44.251 | 15.646 | 1.00 | 64.44 | B |
| 4403 | CG | HIS | B | 1096 | 17.842 | 44.754 | 15.693 | 1.00 | 62.87 | B |
| 4404 | CD2 | HIS | B | 1096 | 16.791 | 44.370 | 16.437 | 1.00 | 63.22 | B |
| 4405 | ND1 | HIS | B | 1096 | 17.379 | 45.779 | 14.902 | 1.00 | 66.64 | B |
| 4406 | CE1 | HIS | B | 1096 | 16.119 | 46.027 | 15.180 | 1.00 | 61.69 | B |
| 4407 | NE2 | HIS | B | 1096 | 15.745 | 45.202 | 16.122 | 1.00 | 61.77 | B |
| 4408 | C | HIS | B | 1096 | 20.869 | 42.330 | 15.352 | 1.00 | 66.77 | B |
| 4409 | O | HIS | B | 1096 | 21.359 | 41.267 | 14.844 | 1.00 | 66.14 | B |
| 4410 | N | ASP | B | 1097 | 21.576 | 43.120 | 16.167 | 1.00 | 68.04 | B |
| 4411 | CA | ASP | B | 1097 | 23.051 | 43.029 | 16.065 | 1.00 | 68.60 | B |
| 4412 | CB | ASP | B | 1097 | 23.703 | 44.437 | 16.185 | 1.00 | 69.50 | B |
| 4413 | CG | ASP | B | 1097 | 24.426 | 44.951 | 14.869 | 1.00 | 68.44 | B |
| 4414 | OD1 | ASP | B | 1097 | 23.780 | 45.127 | 13.737 | 1.00 | 68.44 | B |
| 4415 | OD2 | ASP | B | 1097 | 25.662 | 45.184 | 15.028 | 1.00 | 58.79 | B |
| 4416 | C | ASP | B | 1097 | 23.484 | 42.053 | 17.148 | 1.00 | 69.20 | B |
| 4417 | O | ASP | B | 1097 | 23.447 | 40.820 | 16.944 | 1.00 | 71.01 | B |
| 4418 | N | ASP | B | 1098 | 23.870 | 42.603 | 18.306 | 1.00 | 69.14 | B |
| 4419 | CA | ASP | B | 1098 | 23.835 | 41.929 | 19.600 | 1.00 | 67.30 | B |
| 4420 | CB | ASP | B | 1098 | 24.992 | 42.479 | 20.489 | 1.00 | 67.87 | B |
| 4421 | CG | ASP | B | 1098 | 26.318 | 42.841 | 19.670 | 1.00 | 62.96 | B |
| 4422 | OD1 | ASP | B | 1098 | 26.348 | 43.763 | 18.805 | 1.00 | 66.36 | B |
| 4423 | OD2 | ASP | B | 1098 | 27.362 | 42.278 | 19.954 | 1.00 | 55.30 | B |
| 4424 | C | ASP | B | 1098 | 22.459 | 42.413 | 20.124 | 1.00 | 66.51 | B |
| 4425 | O | ASP | B | 1098 | 22.333 | 42.859 | 21.280 | 1.00 | 64.78 | B |
| 4426 | N | GLY | B | 1099 | 21.463 | 42.352 | 19.210 | 1.00 | 65.78 | B |
| 4427 | CA | GLY | B | 1099 | 20.261 | 43.250 | 19.188 | 1.00 | 64.86 | B |
| 4428 | C | GLY | B | 1099 | 19.357 | 42.200 | 19.620 | 1.00 | 63.59 | B |
| 4429 | O | GLY | B | 1099 | 19.903 | 41.340 | 20.301 | 1.00 | 65.27 | B |
| 4430 | N | ARG | B | 1100 | 18.092 | 42.135 | 19.172 | 1.00 | 61.64 | B |
| 4431 | CA | ARG | B | 1100 | 17.346 | 40.865 | 19.366 | 1.00 | 62.00 | B |
| 4432 | CB | ARG | B | 1100 | 16.746 | 40.624 | 20.811 | 1.00 | 64.43 | B |
| 4433 | CG | ARG | B | 1100 | 17.702 | 39.939 | 21.887 | 1.00 | 57.71 | B |
| 4434 | CD | ARG | B | 1100 | 18.369 | 41.093 | 22.505 | 1.00 | 63.49 | B |
| 4435 | NE | ARG | B | 1100 | 17.529 | 42.318 | 22.606 | 1.00 | 54.85 | B |
| 4436 | CZ | ARG | B | 1100 | 18.046 | 43.498 | 22.861 | 1.00 | 63.24 | B |
| 4437 | NH1 | ARG | B | 1100 | 19.392 | 43.528 | 22.955 | 1.00 | 60.99 | B |
| 4438 | NH2 | ARG | B | 1100 | 17.245 | 44.631 | 22.968 | 1.00 | 67.53 | B |
| 4439 | C | ARG | B | 1100 | 16.423 | 40.440 | 18.208 | 1.00 | 61.11 | B |
| 4440 | O | ARG | B | 1100 | 16.947 | 40.562 | 17.124 | 1.00 | 61.67 | B |
| 4441 | N | TRP | B | 1101 | 15.211 | 39.841 | 18.417 | 1.00 | 56.85 | B |
| 4442 | CA | TRP | B | 1101 | 14.232 | 39.787 | 17.330 | 1.00 | 56.52 | B |
| 4443 | CB | TRP | B | 1101 | 13.377 | 38.473 | 17.136 | 1.00 | 53.72 | B |
| 4444 | CG | TRP | B | 1101 | 13.941 | 37.347 | 16.560 | 1.00 | 52.04 | B |
| 4445 | CD2 | TRP | B | 1101 | 13.332 | 36.069 | 16.416 | 1.00 | 50.51 | B |
| 4446 | CE2 | TRP | B | 1101 | 14.285 | 35.203 | 15.750 | 1.00 | 50.88 | B |
| 4447 | CE3 | TRP | B | 1101 | 12.040 | 35.575 | 16.673 | 1.00 | 52.27 | B |
| 4448 | CD1 | TRP | B | 1101 | 15.246 | 37.217 | 16.048 | 1.00 | 55.64 | B |
| 4449 | NE1 | TRP | B | 1101 | 15.411 | 35.936 | 15.484 | 1.00 | 54.85 | B |
| 4450 | CZ2 | TRP | B | 1101 | 13.994 | 33.895 | 15.411 | 1.00 | 49.64 | B |
| 4451 | CZ3 | TRP | B | 1101 | 11.714 | 34.281 | 16.295 | 1.00 | 48.05 | B |
| 4452 | CH2 | TRP | B | 1101 | 12.692 | 33.447 | 15.669 | 1.00 | 52.95 | B |
| 4453 | C | TRP | B | 1101 | 13.209 | 40.927 | 17.525 | 1.00 | 58.87 | B |
| 4454 | O | TRP | B | 1101 | 13.143 | 41.509 | 18.566 | 1.00 | 61.49 | B |
| 4455 | N | SER | B | 1102 | 12.291 | 41.126 | 16.589 | 1.00 | 58.46 | B |
| 4456 | CA | SER | B | 1102 | 11.606 | 42.304 | 16.622 | 1.00 | 58.61 | B |
| 4457 | CB | SER | B | 1102 | 12.515 | 43.441 | 16.103 | 1.00 | 58.81 | B |
| 4458 | OG | SER | B | 1102 | 11.938 | 44.744 | 16.221 | 1.00 | 58.65 | B |
| 4459 | C | SER | B | 1102 | 10.683 | 41.908 | 15.581 | 1.00 | 58.25 | B |
| 4460 | O | SER | B | 1102 | 11.019 | 42.042 | 14.511 | 1.00 | 56.88 | B |
| 4461 | N | LEU | B | 1103 | 9.527 | 41.387 | 15.975 | 1.00 | 59.97 | B |
| 4462 | CA | LEU | B | 1103 | 8.417 | 41.037 | 15.137 | 1.00 | 59.12 | B |
| 4463 | CB | LEU | B | 1103 | 7.506 | 40.053 | 15.830 | 1.00 | 59.24 | B |
| 4464 | CG | LEU | B | 1103 | 8.127 | 38.799 | 16.509 | 1.00 | 61.64 | B |
| 4465 | CD1 | LEU | B | 1103 | 8.804 | 37.865 | 15.464 | 1.00 | 64.77 | B |
| 4466 | CD2 | LEU | B | 1103 | 9.185 | 39.178 | 17.695 | 1.00 | 58.21 | B |
| 4467 | C | LEU | B | 1103 | 7.649 | 42.257 | 14.883 | 1.00 | 58.65 | B |
| 4468 | O | LEU | B | 1103 | 7.352 | 42.996 | 15.834 | 1.00 | 60.08 | B |
| 4469 | N | GLN | B | 1104 | 7.321 | 42.432 | 13.585 | 1.00 | 56.90 | B |
| 4470 | CA | GLN | B | 1104 | 6.638 | 43.532 | 13.038 | 1.00 | 53.95 | B |
| 4471 | CB | GLN | B | 1104 | 7.680 | 44.312 | 12.351 | 1.00 | 50.13 | B |
| 4472 | CG | GLN | B | 1104 | 6.976 | 45.473 | 11.825 | 1.00 | 55.40 | B |
| 4473 | CD | GLN | B | 1104 | 7.262 | 46.020 | 10.333 | 1.00 | 46.76 | B |
| 4474 | OE1 | GLN | B | 1104 | 7.541 | 45.296 | 9.426 | 1.00 | 41.63 | B |
| 4475 | NE2 | GLN | B | 1104 | 7.076 | 47.341 | 10.172 | 1.00 | 42.70 | B |
| 4476 | C | GLN | B | 1104 | 5.484 | 43.078 | 12.056 | 1.00 | 55.86 | B |
| 4477 | O | GLN | B | 1104 | 5.721 | 42.550 | 10.961 | 1.00 | 60.32 | B |
| 4478 | N | SER | B | 1105 | 4.235 | 43.293 | 12.404 | 1.00 | 55.28 | B |
| 4479 | CA | SER | B | 1105 | 3.099 | 42.825 | 11.654 | 1.00 | 55.14 | B |
| 4480 | CB | SER | B | 1105 | 1.876 | 43.587 | 12.141 | 1.00 | 53.88 | B |
| 4481 | OG | SER | B | 1105 | 2.202 | 44.972 | 12.381 | 1.00 | 55.74 | B |
| 4482 | C | SER | B | 1105 | 3.217 | 43.077 | 10.178 | 1.00 | 56.75 | B |
| 4483 | O | SER | B | 1105 | 3.636 | 44.211 | 9.880 | 1.00 | 59.75 | B |
| 4484 | N | GLU | B | 1106 | 2.870 | 42.122 | 9.250 | 1.00 | 56.54 | B |
| 4485 | CA | GLU | B | 1106 | 3.198 | 42.401 | 7.845 | 1.00 | 56.95 | B |
| 4486 | CB | GLU | B | 1106 | 3.654 | 41.211 | 6.898 | 1.00 | 57.24 | B |
| 4487 | CG | GLU | B | 1106 | 3.923 | 41.720 | 5.361 | 1.00 | 57.17 | B |
| 4488 | CD | GLU | B | 1106 | 5.262 | 41.238 | 4.687 | 1.00 | 60.81 | B |
| 4489 | OE1 | GLU | B | 1106 | 5.507 | 39.997 | 4.626 | 1.00 | 54.56 | B |
| 4490 | OE2 | GLU | B | 1106 | 6.082 | 42.091 | 4.162 | 1.00 | 59.37 | B |
| 4491 | C | GLU | B | 1106 | 2.387 | 43.555 | 7.183 | 1.00 | 55.43 | B |
| 4492 | O | GLU | B | 1106 | 2.966 | 44.553 | 6.799 | 1.00 | 53.46 | B |
| 4493 | N | ALA | B | 1107 | 1.103 | 43.332 | 7.000 | 1.00 | 54.76 | B |
| 4494 | CA | ALA | B | 1107 | 0.154 | 44.415 | 6.847 | 1.00 | 56.78 | B |
| 4495 | CB | ALA | B | 1107 | −1.246 | 43.892 | 7.082 | 1.00 | 54.26 | B |
| 4496 | C | ALA | B | 1107 | 0.453 | 45.583 | 7.896 | 1.00 | 58.38 | B |
| 4497 | O | ALA | B | 1107 | 1.159 | 46.668 | 7.627 | 1.00 | 59.21 | B |
| 4498 | N | HIS | B | 1108 | −0.125 | 45.418 | 9.067 | 1.00 | 55.86 | B |
| 4499 | CA | HIS | B | 1108 | −0.201 | 46.572 | 9.847 | 1.00 | 55.47 | B |
| 4500 | CB | HIS | B | 1108 | −0.961 | 46.210 | 11.010 | 1.00 | 55.70 | B |
| 4501 | CG | HIS | B | 1108 | −2.250 | 45.547 | 10.601 | 1.00 | 54.81 | B |
| 4502 | CD2 | HIS | B | 1108 | −2.956 | 44.523 | 11.131 | 1.00 | 55.20 | B |
| 4503 | ND1 | HIS | B | 1108 | −3.009 | 46.008 | 9.548 | 1.00 | 53.03 | B |
| 4504 | CE1 | HIS | B | 1108 | −4.115 | 45.278 | 9.416 | 1.00 | 44.92 | B |
| 4505 | NE2 | HIS | B | 1108 | −4.117 | 44.385 | 10.384 | 1.00 | 51.22 | B |
| 4506 | C | HIS | B | 1108 | 1.046 | 47.409 | 9.839 | 1.00 | 55.85 | B |
| 4507 | O | HIS | B | 1108 | 0.925 | 48.297 | 9.045 | 1.00 | 57.62 | B |
| 4508 | N | ARG | B | 1109 | 2.248 | 47.028 | 10.409 | 1.00 | 56.47 | B |
| 4509 | CA | ARG | B | 1109 | 3.583 | 47.749 | 10.372 | 1.00 | 57.18 | B |
| 4510 | CB | ARG | B | 1109 | 3.443 | 49.039 | 9.643 | 1.00 | 57.10 | B |
| 4511 | CG | ARG | B | 1109 | 4.760 | 49.796 | 9.195 | 1.00 | 57.51 | B |
| 4512 | CD | ARG | B | 1109 | 5.091 | 49.677 | 7.700 | 1.00 | 49.48 | B |
| 4513 | NE | ARG | B | 1109 | 5.382 | 48.216 | 7.449 | 1.00 | 51.37 | B |
| 4514 | CZ | ARG | B | 1109 | 4.458 | 47.283 | 7.225 | 1.00 | 42.77 | B |
| 4515 | NH1 | ARG | B | 1109 | 3.136 | 47.704 | 7.202 | 1.00 | 27.90 | B |
| 4516 | NH2 | ARG | B | 1109 | 4.911 | 46.000 | 6.964 | 1.00 | 31.24 | B |
| 4517 | C | ARG | B | 1109 | 3.990 | 48.056 | 11.824 | 1.00 | 61.38 | B |
| 4518 | O | ARG | B | 1109 | 4.985 | 48.752 | 12.206 | 1.00 | 60.72 | B |
| 4519 | N | ARG | B | 1110 | 3.184 | 47.423 | 12.657 | 1.00 | 62.26 | B |
| 4520 | CA | ARG | B | 1110 | 3.231 | 47.556 | 14.063 | 1.00 | 62.81 | B |
| 4521 | CB | ARG | B | 1110 | 1.747 | 47.429 | 14.547 | 1.00 | 62.09 | B |
| 4522 | CG | ARG | B | 1110 | 0.696 | 48.465 | 13.933 | 1.00 | 63.18 | B |
| 4523 | CD | ARG | B | 1110 | 0.388 | 49.677 | 14.699 | 1.00 | 51.87 | B |
| 4524 | NE | ARG | B | 1110 | 0.905 | 50.855 | 14.094 | 1.00 | 51.84 | B |
| 4525 | CZ | ARG | B | 1110 | 0.407 | 52.085 | 14.248 | 1.00 | 59.87 | B |
| 4526 | NH1 | ARG | B | 1110 | −0.687 | 52.294 | 14.995 | 1.00 | 63.89 | B |
| 4527 | NH2 | ARG | B | 1110 | 0.938 | 53.129 | 13.590 | 1.00 | 65.78 | B |
| 4528 | C | ARG | B | 1110 | 4.165 | 46.498 | 14.862 | 1.00 | 62.60 | B |
| 4529 | O | ARG | B | 1110 | 3.961 | 45.290 | 14.809 | 1.00 | 57.87 | B |
| 4530 | N | TYR | B | 1111 | 5.068 | 47.030 | 15.720 | 1.00 | 63.48 | B |
| 4531 | CA | TYR | B | 1111 | 6.014 | 46.212 | 16.503 | 1.00 | 61.64 | B |
| 4532 | CB | TYR | B | 1111 | 7.192 | 47.041 | 16.895 | 1.00 | 60.26 | B |
| 4533 | CG | TYR | B | 1111 | 8.003 | 47.471 | 15.685 | 1.00 | 63.00 | B |
| 4534 | CD1 | TYR | B | 1111 | 7.684 | 48.607 | 14.961 | 1.00 | 65.07 | B |
| 4535 | CE1 | TYR | B | 1111 | 8.440 | 49.003 | 13.766 | 1.00 | 61.15 | B |
| 4536 | CD2 | TYR | B | 1111 | 9.064 | 46.700 | 15.192 | 1.00 | 63.67 | B |
| 4537 | CE2 | TYR | B | 1111 | 9.841 | 47.134 | 13.991 | 1.00 | 60.98 | B |
| 4538 | CZ | TYR | B | 1111 | 9.483 | 48.258 | 13.289 | 1.00 | 60.51 | B |
| 4539 | OH | TYR | B | 1111 | 10.206 | 48.677 | 12.142 | 1.00 | 62.40 | B |
| 4540 | C | TYR | B | 1111 | 5.295 | 45.618 | 17.630 | 1.00 | 61.29 | B |
| 4541 | O | TYR | B | 1111 | 4.697 | 46.306 | 18.454 | 1.00 | 62.62 | B |
| 4542 | N | PHE | B | 1112 | 5.238 | 44.294 | 17.583 | 1.00 | 60.83 | B |
| 4543 | CA | PHE | B | 1112 | 4.910 | 43.462 | 18.735 | 1.00 | 58.41 | B |
| 4544 | CB | PHE | B | 1112 | 5.099 | 42.006 | 18.330 | 1.00 | 58.40 | B |
| 4545 | CG | PHE | B | 1112 | 4.792 | 41.026 | 19.392 | 1.00 | 52.91 | B |
| 4546 | CD1 | PHE | B | 1112 | 3.518 | 40.881 | 19.841 | 1.00 | 49.62 | B |
| 4547 | CD2 | PHE | B | 1112 | 5.771 | 40.203 | 19.878 | 1.00 | 56.05 | B |
| 4548 | CE1 | PHE | B | 1112 | 3.153 | 39.955 | 20.823 | 1.00 | 47.42 | B |
| 4549 | CE2 | PHE | B | 1112 | 5.454 | 39.154 | 20.869 | 1.00 | 52.19 | B |
| 4550 | CZ | PHE | B | 1112 | 4.113 | 39.066 | 21.346 | 1.00 | 51.38 | B |
| 4551 | C | PHE | B | 1112 | 5.902 | 43.767 | 19.842 | 1.00 | 58.33 | B |
| 4552 | O | PHE | B | 1112 | 7.161 | 43.793 | 19.717 | 1.00 | 57.25 | B |
| 4553 | N | GLY | B | 1113 | 5.351 | 44.037 | 20.961 | 1.00 | 58.52 | B |
| 4554 | CA | GLY | B | 1113 | 6.283 | 44.219 | 22.028 | 1.00 | 57.43 | B |
| 4555 | C | GLY | B | 1113 | 5.755 | 44.221 | 23.400 | 1.00 | 55.34 | B |
| 4556 | O | GLY | B | 1113 | 6.497 | 44.495 | 24.177 | 1.00 | 57.64 | B |
| 4557 | N | GLY | B | 1114 | 4.556 | 43.783 | 23.716 | 1.00 | 55.29 | B |
| 4558 | CA | GLY | B | 1114 | 3.960 | 44.000 | 25.088 | 1.00 | 57.08 | B |
| 4559 | C | GLY | B | 1114 | 4.688 | 43.860 | 26.468 | 1.00 | 55.49 | B |
| 4560 | O | GLY | B | 1114 | 5.623 | 44.592 | 26.654 | 1.00 | 55.67 | B |
| 4561 | N | THR | B | 1115 | 4.289 | 42.909 | 27.375 | 1.00 | 55.33 | B |
| 4562 | CA | THR | B | 1115 | 4.808 | 42.785 | 28.855 | 1.00 | 53.54 | B |
| 4563 | CB | THR | B | 1115 | 5.049 | 44.194 | 29.546 | 1.00 | 53.46 | B |
| 4564 | OG1 | THR | B | 1115 | 6.452 | 44.519 | 29.738 | 1.00 | 52.93 | B |
| 4565 | CG2 | THR | B | 1115 | 4.182 | 44.440 | 30.776 | 1.00 | 49.59 | B |
| 4566 | C | THR | B | 1115 | 3.721 | 42.047 | 29.639 | 1.00 | 53.08 | B |
| 4567 | O | THR | B | 1115 | 2.484 | 42.446 | 29.624 | 1.00 | 48.68 | B |
| 4568 | N | GLU | B | 1116 | 4.137 | 40.969 | 30.322 | 1.00 | 54.81 | B |
| 4569 | CA | GLU | B | 1116 | 3.233 | 40.305 | 31.293 | 1.00 | 55.17 | B |
| 4570 | CB | GLU | B | 1116 | 3.031 | 41.208 | 32.560 | 1.00 | 53.98 | B |
| 4571 | CG | GLU | B | 1116 | 2.651 | 40.636 | 33.837 | 1.00 | 50.78 | B |
| 4572 | CD | GLU | B | 1116 | 2.643 | 39.101 | 33.881 | 1.00 | 58.26 | B |
| 4573 | OE1 | GLU | B | 1116 | 1.647 | 38.590 | 34.421 | 1.00 | 64.55 | B |
| 4574 | OE2 | GLU | B | 1116 | 3.565 | 38.383 | 33.414 | 1.00 | 54.32 | B |
| 4575 | C | GLU | B | 1116 | 2.001 | 40.267 | 30.554 | 1.00 | 56.92 | B |
| 4576 | O | GLU | B | 1116 | 1.915 | 39.594 | 29.489 | 1.00 | 62.55 | B |
| 4577 | N | ASP | B | 1117 | 1.047 | 41.024 | 30.988 | 1.00 | 56.14 | B |
| 4578 | CA | ASP | B | 1117 | −0.287 | 40.703 | 30.513 | 1.00 | 57.48 | B |
| 4579 | CB | ASP | B | 1117 | −0.961 | 40.648 | 31.862 | 1.00 | 57.01 | B |
| 4580 | CG | ASP | B | 1117 | −1.812 | 41.865 | 32.132 | 1.00 | 51.07 | B |
| 4581 | OD1 | ASP | B | 1117 | −1.380 | 43.035 | 31.879 | 1.00 | 46.51 | B |
| 4582 | OD2 | ASP | B | 1117 | −2.920 | 41.536 | 32.591 | 1.00 | 45.46 | B |
| 4583 | C | ASP | B | 1117 | −1.102 | 41.704 | 29.459 | 1.00 | 57.40 | B |
| 4584 | O | ASP | B | 1117 | −2.312 | 41.634 | 29.262 | 1.00 | 53.01 | B |
| 4585 | N | ARG | B | 1118 | −0.393 | 42.673 | 28.875 | 1.00 | 59.77 | B |
| 4586 | CA | ARG | B | 1118 | −0.961 | 43.858 | 28.209 | 1.00 | 61.30 | B |
| 4587 | CB | ARG | B | 1118 | −1.103 | 45.001 | 29.265 | 1.00 | 60.09 | B |
| 4588 | CG | ARG | B | 1118 | −0.832 | 46.503 | 28.819 | 1.00 | 69.19 | B |
| 4589 | CD | ARG | B | 1118 | −1.997 | 47.656 | 29.363 | 1.00 | 67.69 | B |
| 4590 | NE | ARG | B | 1118 | −3.299 | 46.994 | 29.611 | 1.00 | 82.13 | B |
| 4591 | CZ | ARG | B | 1118 | −4.097 | 46.417 | 28.663 | 1.00 | 88.78 | B |
| 4592 | NH1 | ARG | B | 1118 | −3.779 | 46.386 | 27.331 | 1.00 | 91.32 | B |
| 4593 | NH2 | ARG | B | 1118 | −5.256 | 45.841 | 29.034 | 1.00 | 92.47 | B |
| 4594 | C | ARG | B | 1118 | 0.005 | 44.205 | 27.044 | 1.00 | 61.72 | B |
| 4595 | O | ARG | B | 1118 | 0.391 | 45.439 | 26.912 | 1.00 | 62.81 | B |
| 4596 | N | LEU | B | 1119 | 0.382 | 43.145 | 26.237 | 1.00 | 60.76 | B |
| 4597 | CA | LEU | B | 1119 | 1.103 | 43.221 | 24.854 | 1.00 | 60.81 | B |
| 4598 | CB | LEU | B | 1119 | 1.709 | 41.915 | 24.369 | 1.00 | 59.74 | B |
| 4599 | CG | LEU | B | 1119 | 2.151 | 40.720 | 25.247 | 1.00 | 63.06 | B |
| 4600 | CD1 | LEU | B | 1119 | 1.136 | 40.131 | 26.261 | 1.00 | 61.58 | B |
| 4601 | CD2 | LEU | B | 1119 | 2.403 | 39.649 | 24.208 | 1.00 | 62.02 | B |
| 4602 | C | LEU | B | 1119 | 0.241 | 43.641 | 23.632 | 1.00 | 59.08 | B |
| 4603 | O | LEU | B | 1119 | −0.908 | 43.174 | 23.477 | 1.00 | 54.29 | B |
| 4604 | N | SER | B | 1120 | 0.824 | 44.570 | 22.863 | 1.00 | 58.36 | B |
| 4605 | CA | SER | B | 1120 | 0.201 | 45.127 | 21.681 | 1.00 | 57.61 | B |
| 4606 | CB | SER | B | 1120 | −0.097 | 46.603 | 21.962 | 1.00 | 58.09 | B |
| 4607 | OG | SER | B | 1120 | 0.994 | 47.435 | 21.557 | 1.00 | 56.33 | B |
| 4608 | C | SER | B | 1120 | 1.066 | 45.073 | 20.443 | 1.00 | 56.27 | B |
| 4609 | O | SER | B | 1120 | 2.249 | 45.156 | 20.520 | 1.00 | 55.86 | B |
| 4610 | N | CYS | B | 1121 | 0.489 | 45.022 | 19.269 | 1.00 | 57.53 | B |
| 4611 | CA | CYS | B | 1121 | 1.332 | 45.348 | 18.061 | 1.00 | 58.85 | B |
| 4612 | CB | CYS | B | 1121 | 0.921 | 44.533 | 16.787 | 1.00 | 58.16 | B |
| 4613 | SG | CYS | B | 1121 | 2.355 | 43.884 | 15.785 | 1.00 | 61.16 | B |
| 4614 | C | CYS | B | 1121 | 1.190 | 46.803 | 17.688 | 1.00 | 57.96 | B |
| 4615 | O | CYS | B | 1121 | 0.430 | 47.048 | 16.757 | 1.00 | 59.24 | B |
| 4616 | N | PHE | B | 1122 | 1.750 | 47.773 | 18.403 | 1.00 | 56.13 | B |
| 4617 | CA | PHE | B | 1122 | 1.300 | 49.110 | 18.051 | 1.00 | 55.70 | B |
| 4618 | CB | PHE | B | 1122 | 0.394 | 49.733 | 19.070 | 1.00 | 58.52 | B |
| 4619 | CG | PHE | B | 1122 | −0.642 | 50.812 | 18.480 | 1.00 | 60.08 | B |
| 4620 | CD1 | PHE | B | 1122 | −0.325 | 52.208 | 18.495 | 1.00 | 61.43 | B |
| 4621 | CD2 | PHE | B | 1122 | −1.944 | 50.419 | 18.039 | 1.00 | 58.13 | B |
| 4622 | CE1 | PHE | B | 1122 | −1.218 | 53.148 | 18.035 | 1.00 | 61.52 | B |
| 4623 | CE2 | PHE | B | 1122 | −2.883 | 51.354 | 17.630 | 1.00 | 56.99 | B |
| 4624 | CZ | PHE | B | 1122 | −2.504 | 52.750 | 17.575 | 1.00 | 58.94 | B |
| 4625 | C | PHE | B | 1122 | 2.272 | 50.078 | 17.818 | 1.00 | 54.64 | B |
| 4626 | O | PHE | B | 1122 | 1.895 | 51.070 | 17.332 | 1.00 | 56.54 | B |
| 4627 | N | ALA | B | 1123 | 3.511 | 49.725 | 18.022 | 1.00 | 53.81 | B |
| 4628 | CA | ALA | B | 1123 | 4.602 | 50.599 | 18.357 | 1.00 | 54.89 | B |
| 4629 | CB | ALA | B | 1123 | 5.536 | 49.913 | 19.342 | 1.00 | 48.73 | B |
| 4630 | C | ALA | B | 1123 | 5.283 | 50.780 | 17.026 | 1.00 | 56.46 | B |
| 4631 | O | ALA | B | 1123 | 5.707 | 49.752 | 16.403 | 1.00 | 57.75 | B |
| 4632 | N | GLN | B | 1124 | 5.410 | 52.053 | 16.621 | 1.00 | 56.05 | B |
| 4633 | CA | GLN | B | 1124 | 5.692 | 52.382 | 15.301 | 1.00 | 56.80 | B |
| 4634 | CB | GLN | B | 1124 | 4.747 | 53.461 | 14.811 | 1.00 | 55.61 | B |
| 4635 | CG | GLN | B | 1124 | 5.043 | 54.864 | 15.069 | 1.00 | 56.02 | B |
| 4636 | CD | GLN | B | 1124 | 3.853 | 55.784 | 14.613 | 1.00 | 56.43 | B |
| 4637 | OE1 | GLN | B | 1124 | 2.737 | 55.283 | 14.279 | 1.00 | 62.23 | B |
| 4638 | NE2 | GLN | B | 1124 | 4.085 | 57.097 | 14.549 | 1.00 | 48.49 | B |
| 4639 | C | GLN | B | 1124 | 7.172 | 52.599 | 15.223 | 1.00 | 59.22 | B |
| 4640 | O | GLN | B | 1124 | 7.786 | 53.234 | 14.324 | 1.00 | 60.29 | B |
| 4641 | N | THR | B | 1125 | 7.805 | 52.007 | 16.221 | 1.00 | 61.63 | B |
| 4642 | CA | THR | B | 1125 | 9.300 | 52.113 | 16.403 | 1.00 | 61.49 | B |
| 4643 | CB | THR | B | 1125 | 9.641 | 53.583 | 16.672 | 1.00 | 57.41 | B |
| 4644 | OG1 | THR | B | 1125 | 10.752 | 53.656 | 17.502 | 1.00 | 57.92 | B |
| 4645 | CG2 | THR | B | 1125 | 8.572 | 54.153 | 17.498 | 1.00 | 64.08 | B |
| 4646 | C | THR | B | 1125 | 9.803 | 50.985 | 17.461 | 1.00 | 61.88 | B |
| 4647 | O | THR | B | 1125 | 9.163 | 50.679 | 18.463 | 1.00 | 60.06 | B |
| 4648 | N | VAL | B | 1126 | 10.896 | 50.296 | 17.178 | 1.00 | 64.31 | B |
| 4649 | CA | VAL | B | 1126 | 11.458 | 49.325 | 18.141 | 1.00 | 64.90 | B |
| 4650 | CB | VAL | B | 1126 | 12.822 | 48.694 | 17.673 | 1.00 | 66.92 | B |
| 4651 | CG1 | VAL | B | 1126 | 12.752 | 48.092 | 16.302 | 1.00 | 64.65 | B |
| 4652 | CG2 | VAL | B | 1126 | 14.055 | 49.772 | 17.908 | 1.00 | 68.80 | B |
| 4653 | C | VAL | B | 1126 | 11.788 | 50.123 | 19.439 | 1.00 | 65.08 | B |
| 4654 | O | VAL | B | 1126 | 11.307 | 51.285 | 19.549 | 1.00 | 67.17 | B |
| 4655 | N | SER | B | 1127 | 12.629 | 49.564 | 20.363 | 1.00 | 61.30 | B |
| 4656 | CA | SER | B | 1127 | 12.516 | 50.001 | 21.744 | 1.00 | 56.72 | B |
| 4657 | CB | SER | B | 1127 | 11.148 | 50.559 | 21.930 | 1.00 | 54.62 | B |
| 4658 | OG | SER | B | 1127 | 11.299 | 51.676 | 22.658 | 1.00 | 47.51 | B |
| 4659 | C | SER | B | 1127 | 12.540 | 48.766 | 22.635 | 1.00 | 57.82 | B |
| 4660 | O | SER | B | 1127 | 11.691 | 47.811 | 22.429 | 1.00 | 55.72 | B |
| 4661 | N | PRO | B | 1128 | 13.430 | 48.820 | 23.667 | 1.00 | 55.95 | B |
| 4662 | CD | PRO | B | 1128 | 14.064 | 50.105 | 23.943 | 1.00 | 57.65 | B |
| 4663 | CA | PRO | B | 1128 | 13.701 | 47.963 | 24.682 | 1.00 | 55.35 | B |
| 4664 | CB | PRO | B | 1128 | 13.944 | 48.910 | 25.874 | 1.00 | 56.36 | B |
| 4665 | CG | PRO | B | 1128 | 13.589 | 50.374 | 25.380 | 1.00 | 56.13 | B |
| 4666 | C | PRO | B | 1128 | 12.403 | 47.312 | 24.895 | 1.00 | 57.12 | B |
| 4667 | O | PRO | B | 1128 | 12.360 | 46.133 | 24.901 | 1.00 | 59.52 | B |
| 4668 | N | ALA | B | 1129 | 11.301 | 48.024 | 25.046 | 1.00 | 57.55 | B |
| 4669 | CA | ALA | B | 1129 | 10.015 | 47.346 | 25.364 | 1.00 | 58.55 | B |
| 4670 | CB | ALA | B | 1129 | 8.912 | 48.349 | 25.527 | 1.00 | 56.68 | B |
| 4671 | C | ALA | B | 1129 | 9.590 | 46.211 | 24.399 | 1.00 | 59.79 | B |
| 4672 | O | ALA | B | 1129 | 9.012 | 45.148 | 24.846 | 1.00 | 60.88 | B |
| 4673 | N | GLU | B | 1130 | 9.940 | 46.434 | 23.117 | 1.00 | 60.84 | B |
| 4674 | CA | GLU | B | 1130 | 9.565 | 45.576 | 21.957 | 1.00 | 62.09 | B |
| 4675 | CB | GLU | B | 1130 | 9.232 | 46.344 | 20.607 | 1.00 | 62.04 | B |
| 4676 | CG | GLU | B | 1130 | 8.936 | 47.867 | 20.497 | 1.00 | 60.88 | B |
| 4677 | CD | GLU | B | 1130 | 8.217 | 48.659 | 21.652 | 1.00 | 54.30 | B |
| 4678 | OE1 | GLU | B | 1130 | 8.838 | 49.683 | 22.081 | 1.00 | 55.94 | B |
| 4679 | OE2 | GLU | B | 1130 | 7.072 | 48.363 | 22.050 | 1.00 | 51.60 | B |
| 4680 | C | GLU | B | 1130 | 10.588 | 44.485 | 21.550 | 1.00 | 61.90 | B |
| 4681 | O | GLU | B | 1130 | 10.288 | 43.762 | 20.644 | 1.00 | 62.09 | B |
| 4682 | N | LYS | B | 1131 | 11.756 | 44.428 | 22.225 | 1.00 | 62.20 | B |
| 4683 | CA | LYS | B | 1131 | 12.969 | 43.638 | 21.950 | 1.00 | 61.03 | B |
| 4684 | CB | LYS | B | 1131 | 14.129 | 44.328 | 22.684 | 1.00 | 60.07 | B |
| 4685 | CG | LYS | B | 1131 | 14.598 | 45.594 | 22.107 | 1.00 | 61.44 | B |
| 4686 | CD | LYS | B | 1131 | 14.769 | 45.466 | 20.441 | 1.00 | 62.70 | B |
| 4687 | CE | LYS | B | 1131 | 15.909 | 46.321 | 19.838 | 1.00 | 56.46 | B |
| 4688 | NZ | LYS | B | 1131 | 17.393 | 46.015 | 19.785 | 1.00 | 54.36 | B |
| 4689 | C | LYS | B | 1131 | 12.836 | 42.199 | 22.459 | 1.00 | 61.35 | B |
| 4690 | O | LYS | B | 1131 | 12.733 | 41.896 | 23.665 | 1.00 | 60.12 | B |
| 4691 | N | TRP | B | 1132 | 12.803 | 41.253 | 21.564 | 1.00 | 61.88 | B |
| 4692 | CA | TRP | B | 1132 | 12.424 | 39.919 | 22.111 | 1.00 | 61.77 | B |
| 4693 | CB | TRP | B | 1132 | 11.359 | 39.438 | 21.295 | 1.00 | 56.95 | B |
| 4694 | CG | TRP | B | 1132 | 10.241 | 40.247 | 21.583 | 1.00 | 56.83 | B |
| 4695 | CD2 | TRP | B | 1132 | 9.292 | 39.992 | 22.635 | 1.00 | 52.56 | B |
| 4696 | CE2 | TRP | B | 1132 | 8.309 | 41.002 | 22.577 | 1.00 | 44.07 | B |
| 4697 | CE3 | TRP | B | 1132 | 9.174 | 38.995 | 23.575 | 1.00 | 45.92 | B |
| 4698 | CD1 | TRP | B | 1132 | 9.882 | 41.422 | 20.991 | 1.00 | 47.76 | B |
| 4699 | NE1 | TRP | B | 1132 | 8.667 | 41.876 | 21.589 | 1.00 | 48.76 | B |
| 4700 | CZ2 | TRP | B | 1132 | 7.311 | 41.042 | 23.403 | 1.00 | 45.87 | B |
| 4701 | CZ3 | TRP | B | 1132 | 8.172 | 39.042 | 24.355 | 1.00 | 51.15 | B |
| 4702 | CH2 | TRP | B | 1132 | 7.245 | 40.048 | 24.294 | 1.00 | 50.90 | B |
| 4703 | C | TRP | B | 1132 | 13.402 | 38.834 | 22.019 | 1.00 | 63.25 | B |
| 4704 | O | TRP | B | 1132 | 13.506 | 38.288 | 20.870 | 1.00 | 64.60 | B |
| 4705 | N | SER | B | 1133 | 14.165 | 38.612 | 23.142 | 1.00 | 63.39 | B |
| 4706 | CA | SER | B | 1133 | 15.370 | 37.639 | 23.246 | 1.00 | 61.80 | B |
| 4707 | CB | SER | B | 1133 | 15.845 | 37.518 | 24.764 | 1.00 | 62.44 | B |
| 4708 | OG | SER | B | 1133 | 17.244 | 37.666 | 25.003 | 1.00 | 54.69 | B |
| 4709 | C | SER | B | 1133 | 14.769 | 36.332 | 22.730 | 1.00 | 61.63 | B |
| 4710 | O | SER | B | 1133 | 13.512 | 36.073 | 22.987 | 1.00 | 62.00 | B |
| 4711 | N | VAL | B | 1134 | 15.536 | 35.605 | 21.921 | 1.00 | 59.23 | B |
| 4712 | CA | VAL | B | 1134 | 15.096 | 34.282 | 21.389 | 1.00 | 60.77 | B |
| 4713 | CB | VAL | B | 1134 | 15.593 | 34.179 | 19.946 | 1.00 | 61.56 | B |
| 4714 | CG1 | VAL | B | 1134 | 15.202 | 32.836 | 19.383 | 1.00 | 62.56 | B |
| 4715 | CG2 | VAL | B | 1134 | 14.973 | 35.254 | 19.073 | 1.00 | 63.04 | B |
| 4716 | C | VAL | B | 1134 | 15.629 | 32.955 | 22.087 | 1.00 | 60.93 | B |
| 4717 | O | VAL | B | 1134 | 16.827 | 32.676 | 22.006 | 1.00 | 58.96 | B |
| 4718 | N | HIS | B | 1135 | 14.805 | 32.159 | 22.821 | 1.00 | 63.08 | B |
| 4719 | CA | HIS | B | 1135 | 15.309 | 30.838 | 23.393 | 1.00 | 62.48 | B |
| 4720 | CB | HIS | B | 1135 | 14.916 | 30.486 | 24.792 | 1.00 | 59.25 | B |
| 4721 | CG | HIS | B | 1135 | 16.105 | 30.123 | 25.616 | 1.00 | 63.80 | B |
| 4722 | CD2 | HIS | B | 1135 | 16.785 | 28.954 | 25.741 | 1.00 | 66.22 | B |
| 4723 | ND1 | HIS | B | 1135 | 16.878 | 31.070 | 26.296 | 1.00 | 65.45 | B |
| 4724 | CE1 | HIS | B | 1135 | 17.920 | 30.488 | 26.881 | 1.00 | 62.70 | B |
| 4725 | NE2 | HIS | B | 1135 | 17.899 | 29.203 | 26.540 | 1.00 | 65.56 | B |
| 4726 | C | HIS | B | 1135 | 14.786 | 29.874 | 22.401 | 1.00 | 65.48 | B |
| 4727 | O | HIS | B | 1135 | 13.484 | 29.831 | 22.218 | 1.00 | 66.14 | B |
| 4728 | N | ILE | B | 1136 | 15.713 | 29.293 | 21.576 | 1.00 | 65.18 | B |
| 4729 | CA | ILE | B | 1136 | 15.187 | 28.499 | 20.417 | 1.00 | 64.63 | B |
| 4730 | CB | ILE | B | 1136 | 16.001 | 28.460 | 19.123 | 1.00 | 65.99 | B |
| 4731 | CG2 | ILE | B | 1136 | 15.629 | 27.189 | 18.457 | 1.00 | 71.25 | B |
| 4732 | CG1 | ILE | B | 1136 | 15.480 | 29.456 | 18.084 | 1.00 | 67.67 | B |
| 4733 | CD1 | ILE | B | 1136 | 16.328 | 29.569 | 16.744 | 1.00 | 59.58 | B |
| 4734 | C | ILE | B | 1136 | 15.364 | 27.185 | 20.966 | 1.00 | 62.46 | B |
| 4735 | O | ILE | B | 1136 | 16.414 | 26.953 | 21.541 | 1.00 | 64.20 | B |
| 4736 | N | ALA | B | 1137 | 14.368 | 26.340 | 20.750 | 1.00 | 59.96 | B |
| 4737 | CA | ALA | B | 1137 | 14.252 | 25.125 | 21.473 | 1.00 | 58.34 | B |
| 4738 | CB | ALA | B | 1137 | 13.230 | 25.236 | 22.391 | 1.00 | 56.02 | B |
| 4739 | C | ALA | B | 1137 | 13.950 | 24.081 | 20.500 | 1.00 | 59.79 | B |
| 4740 | O | ALA | B | 1137 | 12.887 | 23.489 | 20.552 | 1.00 | 60.32 | B |
| 4741 | N | MET | B | 1138 | 14.871 | 23.867 | 19.547 | 1.00 | 62.18 | B |
| 4742 | CA | MET | B | 1138 | 14.799 | 22.715 | 18.621 | 1.00 | 63.70 | B |
| 4743 | CB | MET | B | 1138 | 13.936 | 22.926 | 17.390 | 1.00 | 64.40 | B |
| 4744 | CG | MET | B | 1138 | 14.554 | 23.986 | 16.417 | 1.00 | 68.33 | B |
| 4745 | SD | MET | B | 1138 | 13.607 | 25.483 | 15.775 | 1.00 | 67.45 | B |
| 4746 | CE | MET | B | 1138 | 15.165 | 26.343 | 15.370 | 1.00 | 66.79 | B |
| 4747 | C | MET | B | 1138 | 16.223 | 22.447 | 18.273 | 1.00 | 63.11 | B |
| 4748 | O | MET | B | 1138 | 17.164 | 23.184 | 18.728 | 1.00 | 60.87 | B |
| 4749 | N | HIS | B | 1139 | 16.376 | 21.352 | 17.524 | 1.00 | 61.38 | B |
| 4750 | CA | HIS | B | 1139 | 17.545 | 20.525 | 17.652 | 1.00 | 59.71 | B |
| 4751 | CB | HIS | B | 1139 | 17.065 | 19.040 | 17.575 | 1.00 | 59.49 | B |
| 4752 | CG | HIS | B | 1139 | 18.142 | 17.986 | 17.600 | 1.00 | 57.77 | B |
| 4753 | CD2 | HIS | B | 1139 | 18.182 | 16.827 | 18.302 | 1.00 | 44.62 | B |
| 4754 | ND1 | HIS | B | 1139 | 19.317 | 18.026 | 16.819 | 1.00 | 59.84 | B |
| 4755 | CE1 | HIS | B | 1139 | 20.034 | 16.943 | 17.095 | 1.00 | 53.97 | B |
| 4756 | NE2 | HIS | B | 1139 | 19.370 | 16.216 | 17.999 | 1.00 | 45.49 | B |
| 4757 | C | HIS | B | 1139 | 18.666 | 21.003 | 16.628 | 1.00 | 60.77 | B |
| 4758 | O | HIS | B | 1139 | 18.689 | 20.807 | 15.462 | 1.00 | 60.56 | B |
| 4759 | N | PRO | B | 1140 | 19.718 | 21.540 | 17.155 | 1.00 | 61.79 | B |
| 4760 | CD | PRO | B | 1140 | 20.021 | 21.291 | 18.579 | 1.00 | 62.24 | B |
| 4761 | CA | PRO | B | 1140 | 20.809 | 22.167 | 16.473 | 1.00 | 61.05 | B |
| 4762 | CB | PRO | B | 1140 | 21.939 | 21.894 | 17.441 | 1.00 | 61.98 | B |
| 4763 | CG | PRO | B | 1140 | 21.334 | 20.778 | 18.480 | 1.00 | 60.14 | B |
| 4764 | C | PRO | B | 1140 | 21.172 | 21.432 | 15.186 | 1.00 | 61.54 | B |
| 4765 | O | PRO | B | 1140 | 21.664 | 22.017 | 14.175 | 1.00 | 59.47 | B |
| 4766 | N | GLN | B | 1141 | 21.050 | 20.108 | 15.265 | 1.00 | 61.90 | B |
| 4767 | CA | GLN | B | 1141 | 21.728 | 19.377 | 14.192 | 1.00 | 60.97 | B |
| 4768 | CB | GLN | B | 1141 | 22.260 | 18.020 | 14.642 | 1.00 | 61.23 | B |
| 4769 | CG | GLN | B | 1141 | 23.520 | 18.142 | 15.537 | 1.00 | 62.58 | B |
| 4770 | CD | GLN | B | 1141 | 23.893 | 16.770 | 16.148 | 1.00 | 64.02 | B |
| 4771 | OE1 | GLN | B | 1141 | 24.694 | 16.658 | 17.114 | 1.00 | 63.81 | B |
| 4772 | NE2 | GLN | B | 1141 | 23.310 | 15.709 | 15.570 | 1.00 | 57.36 | B |
| 4773 | C | GLN | B | 1141 | 20.650 | 19.312 | 13.173 | 1.00 | 57.38 | B |
| 4774 | O | GLN | B | 1141 | 19.491 | 18.991 | 13.556 | 1.00 | 55.16 | B |
| 4775 | N | VAL | B | 1142 | 21.022 | 19.732 | 11.952 | 1.00 | 52.38 | B |
| 4776 | CA | VAL | B | 1142 | 20.124 | 19.871 | 10.885 | 1.00 | 50.13 | B |
| 4777 | CB | VAL | B | 1142 | 19.520 | 21.268 | 10.936 | 1.00 | 49.58 | B |
| 4778 | CG1 | VAL | B | 1142 | 18.747 | 21.429 | 12.237 | 1.00 | 47.82 | B |
| 4779 | CG2 | VAL | B | 1142 | 20.526 | 22.354 | 10.759 | 1.00 | 48.71 | B |
| 4780 | C | VAL | B | 1142 | 21.067 | 19.652 | 9.812 | 1.00 | 51.77 | B |
| 4781 | O | VAL | B | 1142 | 22.178 | 19.769 | 10.138 | 1.00 | 55.26 | B |
| 4782 | N | ASN | B | 1143 | 20.721 | 19.205 | 8.599 | 1.00 | 52.69 | B |
| 4783 | CA | ASN | B | 1143 | 21.507 | 19.485 | 7.384 | 1.00 | 50.91 | B |
| 4784 | CB | ASN | B | 1143 | 20.969 | 18.619 | 6.281 | 1.00 | 50.34 | B |
| 4785 | CG | ASN | B | 1143 | 21.645 | 17.370 | 6.145 | 1.00 | 53.45 | B |
| 4786 | OD1 | ASN | B | 1143 | 21.606 | 16.762 | 5.095 | 1.00 | 50.95 | B |
| 4787 | ND2 | ASN | B | 1143 | 22.406 | 16.986 | 7.185 | 1.00 | 64.92 | B |
| 4788 | C | ASN | B | 1143 | 21.226 | 20.944 | 6.832 | 1.00 | 52.44 | B |
| 4789 | O | ASN | B | 1143 | 20.249 | 21.589 | 7.249 | 1.00 | 49.33 | B |
| 4790 | N | ILE | B | 1144 | 22.017 | 21.370 | 5.787 | 1.00 | 52.91 | B |
| 4791 | CA | ILE | B | 1144 | 21.897 | 22.641 | 5.070 | 1.00 | 50.94 | B |
| 4792 | CB | ILE | B | 1144 | 22.936 | 23.652 | 5.676 | 1.00 | 53.76 | B |
| 4793 | CG2 | ILE | B | 1144 | 23.021 | 25.068 | 4.900 | 1.00 | 50.93 | B |
| 4794 | CG1 | ILE | B | 1144 | 22.426 | 24.096 | 7.085 | 1.00 | 50.20 | B |
| 4795 | CD1 | ILE | B | 1144 | 23.210 | 25.253 | 7.526 | 1.00 | 41.86 | B |
| 4796 | C | ILE | B | 1144 | 21.948 | 22.583 | 3.587 | 1.00 | 50.90 | B |
| 4797 | O | ILE | B | 1144 | 22.759 | 21.963 | 3.051 | 1.00 | 46.18 | B |
| 4798 | N | TYR | B | 1145 | 20.988 | 23.215 | 2.916 | 1.00 | 56.07 | B |
| 4799 | CA | TYR | B | 1145 | 20.718 | 22.992 | 1.441 | 1.00 | 58.11 | B |
| 4800 | CB | TYR | B | 1145 | 19.700 | 21.835 | 1.235 | 1.00 | 56.68 | B |
| 4801 | CG | TYR | B | 1145 | 19.119 | 21.718 | −0.258 | 1.00 | 59.62 | B |
| 4802 | CD1 | TYR | B | 1145 | 19.821 | 20.949 | −1.236 | 1.00 | 58.76 | B |
| 4803 | CE1 | TYR | B | 1145 | 19.422 | 20.896 | −2.620 | 1.00 | 59.07 | B |
| 4804 | CD2 | TYR | B | 1145 | 17.894 | 22.405 | −0.693 | 1.00 | 55.32 | B |
| 4805 | CE2 | TYR | B | 1145 | 17.479 | 22.314 | −2.029 | 1.00 | 54.07 | B |
| 4806 | CZ | TYR | B | 1145 | 18.252 | 21.567 | −3.021 | 1.00 | 59.67 | B |
| 4807 | OH | TYR | B | 1145 | 17.915 | 21.416 | −4.416 | 1.00 | 54.79 | B |
| 4808 | C | TYR | B | 1145 | 20.152 | 24.270 | 0.712 | 1.00 | 60.40 | B |
| 4809 | O | TYR | B | 1145 | 19.004 | 24.741 | 1.065 | 1.00 | 61.44 | B |
| 4810 | N | SER | B | 1146 | 20.884 | 24.833 | −0.283 | 1.00 | 62.03 | B |
| 4811 | CA | SER | B | 1146 | 20.356 | 26.052 | −1.037 | 1.00 | 63.22 | B |
| 4812 | CB | SER | B | 1146 | 21.144 | 27.384 | −0.702 | 1.00 | 65.08 | B |
| 4813 | OG | SER | B | 1146 | 20.947 | 27.840 | 0.685 | 1.00 | 64.42 | B |
| 4814 | C | SER | B | 1146 | 20.032 | 25.862 | −2.562 | 1.00 | 63.73 | B |
| 4815 | O | SER | B | 1146 | 20.766 | 25.199 | −3.263 | 1.00 | 65.89 | B |
| 4816 | N | VAL | B | 1147 | 18.963 | 26.442 | −3.086 | 1.00 | 63.15 | B |
| 4817 | CA | VAL | B | 1147 | 18.670 | 26.267 | −4.526 | 1.00 | 63.23 | B |
| 4818 | CB | VAL | B | 1147 | 17.213 | 26.653 | −4.773 | 1.00 | 63.38 | B |
| 4819 | CG1 | VAL | B | 1147 | 16.937 | 27.258 | −6.156 | 1.00 | 61.13 | B |
| 4820 | CG2 | VAL | B | 1147 | 16.232 | 25.427 | −4.382 | 1.00 | 65.27 | B |
| 4821 | C | VAL | B | 1147 | 19.595 | 27.207 | −5.255 | 1.00 | 64.27 | B |
| 4822 | O | VAL | B | 1147 | 20.068 | 26.966 | −6.447 | 1.00 | 63.13 | B |
| 4823 | N | THR | B | 1148 | 19.877 | 28.292 | −4.515 | 1.00 | 64.92 | B |
| 4824 | CA | THR | B | 1148 | 20.955 | 29.176 | −4.926 | 1.00 | 66.58 | B |
| 4825 | CB | THR | B | 1148 | 21.145 | 30.438 | −3.889 | 1.00 | 67.44 | B |
| 4826 | OG1 | THR | B | 1148 | 19.918 | 31.240 | −3.860 | 1.00 | 68.55 | B |
| 4827 | CG2 | THR | B | 1148 | 22.246 | 31.482 | −4.345 | 1.00 | 65.23 | B |
| 4828 | C | THR | B | 1148 | 22.146 | 28.217 | −5.366 | 1.00 | 65.87 | B |
| 4829 | O | THR | B | 1148 | 22.484 | 27.982 | −6.557 | 1.00 | 67.41 | B |
| 4830 | N | ARG | B | 1149 | 22.640 | 27.506 | −4.418 | 1.00 | 64.59 | B |
| 4831 | CA | ARG | B | 1149 | 23.613 | 26.532 | −4.730 | 1.00 | 63.02 | B |
| 4832 | CB | ARG | B | 1149 | 24.478 | 26.316 | −3.483 | 1.00 | 62.83 | B |
| 4833 | CG | ARG | B | 1149 | 25.729 | 27.267 | −3.502 | 1.00 | 56.37 | B |
| 4834 | CD | ARG | B | 1149 | 26.950 | 26.395 | −4.129 | 1.00 | 58.52 | B |
| 4835 | NE | ARG | B | 1149 | 28.096 | 27.284 | −4.437 | 1.00 | 57.19 | B |
| 4836 | CZ | ARG | B | 1149 | 27.965 | 28.602 | −4.472 | 1.00 | 55.88 | B |
| 4837 | NH1 | ARG | B | 1149 | 26.733 | 29.168 | −4.193 | 1.00 | 50.52 | B |
| 4838 | NH2 | ARG | B | 1149 | 29.024 | 29.302 | −4.809 | 1.00 | 57.08 | B |
| 4839 | C | ARG | B | 1149 | 23.032 | 25.238 | −5.295 | 1.00 | 64.29 | B |
| 4840 | O | ARG | B | 1149 | 23.674 | 24.607 | −6.174 | 1.00 | 64.81 | B |
| 4841 | N | LYS | B | 1150 | 21.847 | 24.810 | −4.847 | 1.00 | 64.52 | B |
| 4842 | CA | LYS | B | 1150 | 21.120 | 23.741 | −5.577 | 1.00 | 64.58 | B |
| 4843 | CB | LYS | B | 1150 | 21.319 | 23.856 | −7.134 | 1.00 | 66.67 | B |
| 4844 | CG | LYS | B | 1150 | 20.200 | 24.642 | −7.944 | 1.00 | 69.65 | B |
| 4845 | CD | LYS | B | 1150 | 19.189 | 23.602 | −8.590 | 1.00 | 77.96 | B |
| 4846 | CE | LYS | B | 1150 | 18.198 | 22.721 | −7.546 | 1.00 | 76.91 | B |
| 4847 | NZ | LYS | B | 1150 | 16.734 | 22.442 | −7.983 | 1.00 | 67.36 | B |
| 4848 | C | LYS | B | 1150 | 21.583 | 22.377 | −5.091 | 1.00 | 63.51 | B |
| 4849 | O | LYS | B | 1150 | 20.807 | 21.361 | −5.159 | 1.00 | 65.74 | B |
| 4850 | N | ARG | B | 1151 | 22.780 | 22.369 | −4.522 | 1.00 | 59.06 | B |
| 4851 | CA | ARG | B | 1151 | 23.465 | 21.175 | −4.116 | 1.00 | 56.59 | B |
| 4852 | CB | ARG | B | 1151 | 24.835 | 21.224 | −4.641 | 1.00 | 54.87 | B |
| 4853 | CG | ARG | B | 1151 | 25.039 | 20.469 | −5.691 | 1.00 | 50.26 | B |
| 4854 | CD | ARG | B | 1151 | 26.363 | 20.804 | −5.920 | 1.00 | 45.15 | B |
| 4855 | NE | ARG | B | 1151 | 26.864 | 20.392 | −7.230 | 1.00 | 51.00 | B |
| 4856 | CZ | ARG | B | 1151 | 27.015 | 21.210 | −8.282 | 1.00 | 48.46 | B |
| 4857 | NH1 | ARG | B | 1151 | 26.647 | 22.516 | −8.167 | 1.00 | 44.69 | B |
| 4858 | NH2 | ARG | B | 1151 | 27.683 | 20.770 | −9.405 | 1.00 | 45.95 | B |
| 4859 | C | ARG | B | 1151 | 23.696 | 21.210 | −2.609 | 1.00 | 58.29 | B |
| 4860 | O | ARG | B | 1151 | 23.350 | 22.285 | −1.999 | 1.00 | 57.14 | B |
| 4861 | N | TYR | B | 1152 | 24.303 | 20.091 | −2.072 | 1.00 | 57.01 | B |
| 4862 | CA | TYR | B | 1152 | 24.416 | 19.804 | −0.624 | 1.00 | 56.46 | B |
| 4863 | CB | TYR | B | 1152 | 24.122 | 18.276 | −0.147 | 1.00 | 56.05 | B |
| 4864 | CG | TYR | B | 1152 | 22.609 | 18.039 | 0.121 | 1.00 | 50.06 | B |
| 4865 | CD1 | TYR | B | 1152 | 21.775 | 17.572 | −0.899 | 1.00 | 51.65 | B |
| 4866 | CE1 | TYR | B | 1152 | 20.364 | 17.434 | −0.751 | 1.00 | 44.50 | B |
| 4867 | CD2 | TYR | B | 1152 | 22.043 | 18.375 | 1.305 | 1.00 | 38.75 | B |
| 4868 | CE2 | TYR | B | 1152 | 20.616 | 18.309 | 1.458 | 1.00 | 45.21 | B |
| 4869 | CZ | TYR | B | 1152 | 19.782 | 17.830 | 0.378 | 1.00 | 45.80 | B |
| 4870 | OH | TYR | B | 1152 | 18.401 | 17.673 | 0.513 | 1.00 | 39.69 | B |
| 4871 | C | TYR | B | 1152 | 25.787 | 20.163 | −0.193 | 1.00 | 58.08 | B |
| 4872 | O | TYR | B | 1152 | 26.814 | 19.871 | −0.895 | 1.00 | 57.18 | B |
| 4873 | N | ALA | B | 1153 | 25.793 | 20.725 | 1.027 | 1.00 | 59.39 | B |
| 4874 | CA | ALA | B | 1153 | 26.974 | 21.396 | 1.535 | 1.00 | 60.62 | B |
| 4875 | CB | ALA | B | 1153 | 26.561 | 22.791 | 1.956 | 1.00 | 58.58 | B |
| 4876 | C | ALA | B | 1153 | 27.664 | 20.693 | 2.696 | 1.00 | 61.34 | B |
| 4877 | O | ALA | B | 1153 | 27.119 | 20.867 | 3.795 | 1.00 | 64.42 | B |
| 4878 | N | HIS | B | 1154 | 28.864 | 20.053 | 2.506 | 1.00 | 62.19 | B |
| 4879 | CA | HIS | B | 1154 | 29.574 | 19.087 | 3.475 | 1.00 | 62.48 | B |
| 4880 | CB | HIS | B | 1154 | 29.869 | 17.726 | 2.849 | 1.00 | 63.02 | B |
| 4881 | CG | HIS | B | 1154 | 30.807 | 17.746 | 1.674 | 1.00 | 61.92 | B |
| 4882 | CD2 | HIS | B | 1154 | 31.769 | 16.849 | 1.308 | 1.00 | 63.04 | B |
| 4883 | ND1 | HIS | B | 1154 | 30.694 | 18.644 | 0.599 | 1.00 | 58.83 | B |
| 4884 | CE1 | HIS | B | 1154 | 31.613 | 18.347 | −0.325 | 1.00 | 52.73 | B |
| 4885 | NE2 | HIS | B | 1154 | 32.264 | 17.245 | 0.066 | 1.00 | 55.83 | B |
| 4886 | C | HIS | B | 1154 | 30.940 | 19.426 | 3.786 | 1.00 | 63.46 | B |
| 4887 | O | HIS | B | 1154 | 31.567 | 19.973 | 2.908 | 1.00 | 62.28 | B |
| 4888 | N | LEU | B | 1155 | 31.468 | 18.960 | 4.950 | 1.00 | 64.83 | B |
| 4889 | CA | LEU | B | 1155 | 32.705 | 19.609 | 5.585 | 1.00 | 62.12 | B |
| 4890 | CB | LEU | B | 1155 | 32.861 | 19.397 | 7.141 | 1.00 | 62.68 | B |
| 4891 | CG | LEU | B | 1155 | 33.233 | 20.566 | 8.263 | 1.00 | 63.40 | B |
| 4892 | CD1 | LEU | B | 1155 | 32.465 | 21.762 | 8.216 | 1.00 | 56.90 | B |
| 4893 | CD2 | LEU | B | 1155 | 33.334 | 20.297 | 9.847 | 1.00 | 57.70 | B |
| 4894 | C | LEU | B | 1155 | 33.924 | 19.259 | 4.876 | 1.00 | 62.90 | B |
| 4895 | O | LEU | B | 1155 | 34.765 | 18.683 | 5.488 | 1.00 | 65.20 | B |
| 4896 | N | SER | B | 1156 | 34.047 | 19.626 | 3.610 | 1.00 | 63.14 | B |
| 4897 | CA | SER | B | 1156 | 35.132 | 19.258 | 2.727 | 1.00 | 66.60 | B |
| 4898 | CB | SER | B | 1156 | 35.624 | 20.527 | 2.140 | 1.00 | 68.01 | B |
| 4899 | OG | SER | B | 1156 | 35.525 | 20.433 | 0.714 | 1.00 | 76.63 | B |
| 4900 | C | SER | B | 1156 | 36.398 | 18.626 | 3.277 | 1.00 | 69.43 | B |
| 4901 | O | SER | B | 1156 | 36.928 | 18.961 | 4.421 | 1.00 | 69.50 | B |
| 4902 | N | ALA | B | 1157 | 36.987 | 17.791 | 2.435 | 1.00 | 70.75 | B |
| 4903 | CA | ALA | B | 1157 | 38.205 | 17.099 | 2.876 | 1.00 | 72.13 | B |
| 4904 | CB | ALA | B | 1157 | 38.414 | 15.941 | 1.983 | 1.00 | 73.34 | B |
| 4905 | C | ALA | B | 1157 | 39.553 | 17.952 | 3.013 | 1.00 | 73.68 | B |
| 4906 | O | ALA | B | 1157 | 39.615 | 19.056 | 3.677 | 1.00 | 72.33 | B |
| 4907 | N | ARG | B | 1158 | 40.616 | 17.406 | 2.372 | 1.00 | 74.32 | B |
| 4908 | CA | ARG | B | 1158 | 41.915 | 18.085 | 2.153 | 1.00 | 73.77 | B |
| 4909 | CB | ARG | B | 1158 | 42.425 | 17.959 | 0.695 | 1.00 | 73.11 | B |
| 4910 | CG | ARG | B | 1158 | 42.450 | 16.543 | −0.014 | 1.00 | 73.51 | B |
| 4911 | CD | ARG | B | 1158 | 43.490 | 15.501 | 0.619 | 1.00 | 72.93 | B |
| 4912 | NE | ARG | B | 1158 | 42.833 | 14.222 | 0.890 | 1.00 | 75.12 | B |
| 4913 | CZ | ARG | B | 1158 | 43.370 | 13.141 | 1.458 | 1.00 | 78.02 | B |
| 4914 | NH1 | ARG | B | 1158 | 44.650 | 13.148 | 1.877 | 1.00 | 77.66 | B |
| 4915 | NH2 | ARG | B | 1158 | 42.584 | 12.035 | 1.587 | 1.00 | 75.36 | B |
| 4916 | C | ARG | B | 1158 | 41.768 | 19.545 | 2.497 | 1.00 | 74.10 | B |
| 4917 | O | ARG | B | 1158 | 42.263 | 19.991 | 3.541 | 1.00 | 74.12 | B |
| 4918 | N | PRO | B | 1159 | 41.053 | 20.304 | 1.627 | 1.00 | 75.39 | B |
| 4919 | CD | PRO | B | 1159 | 40.492 | 19.970 | 0.285 | 1.00 | 75.32 | B |
| 4920 | CA | PRO | B | 1159 | 40.648 | 21.676 | 2.059 | 1.00 | 75.22 | B |
| 4921 | CB | PRO | B | 1159 | 39.453 | 21.966 | 1.109 | 1.00 | 75.69 | B |
| 4922 | CG | PRO | B | 1159 | 39.182 | 20.619 | 0.292 | 1.00 | 72.50 | B |
| 4923 | C | PRO | B | 1159 | 40.342 | 21.871 | 3.640 | 1.00 | 75.69 | B |
| 4924 | O | PRO | B | 1159 | 39.285 | 21.397 | 4.180 | 1.00 | 74.86 | B |
| 4925 | N | ALA | B | 1160 | 41.327 | 22.483 | 4.346 | 1.00 | 75.34 | B |
| 4926 | CA | ALA | B | 1160 | 41.298 | 22.886 | 5.857 | 1.00 | 74.29 | B |
| 4927 | CB | ALA | B | 1160 | 42.616 | 23.582 | 6.209 | 1.00 | 73.88 | B |
| 4928 | C | ALA | B | 1160 | 40.180 | 23.782 | 6.478 | 1.00 | 72.51 | B |
| 4929 | O | ALA | B | 1160 | 40.182 | 25.002 | 6.276 | 1.00 | 71.76 | B |
| 4930 | N | ASP | B | 1161 | 39.273 | 23.246 | 7.277 | 1.00 | 72.31 | B |
| 4931 | CA | ASP | B | 1161 | 38.122 | 24.097 | 7.648 | 1.00 | 71.81 | B |
| 4932 | CB | ASP | B | 1161 | 38.682 | 25.462 | 8.144 | 1.00 | 73.28 | B |
| 4933 | CG | ASP | B | 1161 | 37.592 | 26.495 | 8.303 | 1.00 | 76.86 | B |
| 4934 | OD1 | ASP | B | 1161 | 36.412 | 26.002 | 8.471 | 1.00 | 76.69 | B |
| 4935 | OD2 | ASP | B | 1161 | 37.898 | 27.748 | 8.171 | 1.00 | 78.61 | B |
| 4936 | C | ASP | B | 1161 | 37.236 | 24.362 | 6.436 | 1.00 | 70.67 | B |
| 4937 | O | ASP | B | 1161 | 37.826 | 24.534 | 5.345 | 1.00 | 72.10 | B |
| 4938 | N | GLU | B | 1162 | 35.884 | 24.453 | 6.544 | 1.00 | 68.43 | B |
| 4939 | CA | GLU | B | 1162 | 35.033 | 24.717 | 5.258 | 1.00 | 67.81 | B |
| 4940 | CB | GLU | B | 1162 | 35.824 | 24.336 | 3.933 | 1.00 | 67.10 | B |
| 4941 | CG | GLU | B | 1162 | 35.502 | 25.145 | 2.645 | 1.00 | 67.98 | B |
| 4942 | CD | GLU | B | 1162 | 36.704 | 25.521 | 1.765 | 1.00 | 68.89 | B |
| 4943 | OE1 | GLU | B | 1162 | 37.474 | 24.560 | 1.321 | 1.00 | 64.08 | B |
| 4944 | OE2 | GLU | B | 1162 | 36.855 | 26.801 | 1.492 | 1.00 | 70.67 | B |
| 4945 | C | GLU | B | 1162 | 33.561 | 24.197 | 5.052 | 1.00 | 65.90 | B |
| 4946 | O | GLU | B | 1162 | 32.803 | 23.814 | 5.942 | 1.00 | 66.47 | B |
| 4947 | N | ILE | B | 1163 | 33.120 | 24.177 | 3.821 | 1.00 | 64.15 | B |
| 4948 | CA | ILE | B | 1163 | 31.744 | 23.877 | 3.616 | 1.00 | 62.73 | B |
| 4949 | CB | ILE | B | 1163 | 30.784 | 24.722 | 4.470 | 1.00 | 62.40 | B |
| 4950 | CG2 | ILE | B | 1163 | 29.282 | 23.990 | 4.595 | 1.00 | 63.74 | B |
| 4951 | CG1 | ILE | B | 1163 | 31.322 | 24.904 | 5.846 | 1.00 | 58.15 | B |
| 4952 | CD1 | ILE | B | 1163 | 30.446 | 25.796 | 6.627 | 1.00 | 59.20 | B |
| 4953 | C | ILE | B | 1163 | 31.285 | 23.734 | 2.132 | 1.00 | 62.12 | B |
| 4954 | O | ILE | B | 1163 | 30.075 | 24.005 | 1.808 | 1.00 | 60.21 | B |
| 4955 | N | ALA | B | 1164 | 32.203 | 23.163 | 1.319 | 1.00 | 60.36 | B |
| 4956 | CA | ALA | B | 1164 | 31.903 | 22.788 | −0.105 | 1.00 | 61.45 | B |
| 4957 | CB | ALA | B | 1164 | 32.968 | 21.877 | −0.615 | 1.00 | 63.49 | B |
| 4958 | C | ALA | B | 1164 | 30.485 | 22.206 | −0.418 | 1.00 | 60.79 | B |
| 4959 | O | ALA | B | 1164 | 29.973 | 21.170 | 0.185 | 1.00 | 61.99 | B |
| 4960 | N | VAL | B | 1165 | 29.813 | 22.922 | −1.276 | 1.00 | 56.08 | B |
| 4961 | CA | VAL | B | 1165 | 28.451 | 22.639 | −1.425 | 1.00 | 54.86 | B |
| 4962 | CB | VAL | B | 1165 | 27.596 | 23.917 | −1.306 | 1.00 | 54.58 | B |
| 4963 | CG1 | VAL | B | 1165 | 26.021 | 23.577 | −1.511 | 1.00 | 53.27 | B |
| 4964 | CG2 | VAL | B | 1165 | 27.824 | 24.546 | −0.094 | 1.00 | 51.69 | B |
| 4965 | C | VAL | B | 1165 | 28.279 | 21.981 | −2.794 | 1.00 | 54.82 | B |
| 4966 | O | VAL | B | 1165 | 27.506 | 22.480 | −3.610 | 1.00 | 54.00 | B |
| 4967 | N | ASP | B | 1166 | 28.991 | 20.843 | −3.009 | 1.00 | 57.17 | B |
| 4968 | CA | ASP | B | 1166 | 29.187 | 20.102 | −4.338 | 1.00 | 57.21 | B |
| 4969 | CB | ASP | B | 1166 | 30.647 | 20.182 | −4.938 | 1.00 | 55.19 | B |
| 4970 | CG | ASP | B | 1166 | 31.739 | 19.435 | −4.023 | 1.00 | 64.88 | B |
| 4971 | OD1 | ASP | B | 1166 | 31.488 | 19.202 | −2.788 | 1.00 | 74.98 | B |
| 4972 | OD2 | ASP | B | 1166 | 32.892 | 19.140 | −4.452 | 1.00 | 65.35 | B |
| 4973 | C | ASP | B | 1166 | 28.740 | 18.652 | −4.232 | 1.00 | 57.23 | B |
| 4974 | O | ASP | B | 1166 | 29.475 | 17.701 | −4.568 | 1.00 | 59.84 | B |
| 4975 | N | ARG | B | 1167 | 27.560 | 18.398 | −3.758 | 1.00 | 57.11 | B |
| 4976 | CA | ARG | B | 1167 | 27.305 | 16.949 | −3.643 | 1.00 | 56.20 | B |
| 4977 | CB | ARG | B | 1167 | 28.062 | 16.398 | −2.378 | 1.00 | 56.90 | B |
| 4978 | CG | ARG | B | 1167 | 27.619 | 16.966 | −1.048 | 1.00 | 55.74 | B |
| 4979 | CD | ARG | B | 1167 | 27.198 | 15.777 | −0.203 | 1.00 | 63.75 | B |
| 4980 | NE | ARG | B | 1167 | 28.328 | 14.923 | 0.272 | 1.00 | 68.30 | B |
| 4981 | CZ | ARG | B | 1167 | 28.517 | 14.455 | 1.535 | 1.00 | 65.43 | B |
| 4982 | NH1 | ARG | B | 1167 | 27.622 | 14.694 | 2.501 | 1.00 | 59.52 | B |
| 4983 | NH2 | ARG | B | 1167 | 29.634 | 13.748 | 1.827 | 1.00 | 65.11 | B |
| 4984 | C | ARG | B | 1167 | 25.808 | 16.688 | −3.662 | 1.00 | 56.12 | B |
| 4985 | O | ARG | B | 1167 | 25.003 | 17.467 | −3.118 | 1.00 | 56.47 | B |
| 4986 | N | ASP | B | 1168 | 25.381 | 15.649 | −4.353 | 1.00 | 57.27 | B |
| 4987 | CA | ASP | B | 1168 | 23.945 | 15.553 | −4.561 | 1.00 | 58.14 | B |
| 4988 | CB | ASP | B | 1168 | 23.366 | 14.498 | −5.608 | 1.00 | 57.24 | B |
| 4989 | CG | ASP | B | 1168 | 24.030 | 13.037 | −5.591 | 1.00 | 57.06 | B |
| 4990 | OD1 | ASP | B | 1168 | 25.294 | 12.940 | −5.400 | 1.00 | 54.25 | B |
| 4991 | OD2 | ASP | B | 1168 | 23.274 | 11.996 | −5.928 | 1.00 | 47.05 | B |
| 4992 | C | ASP | B | 1168 | 23.317 | 15.349 | −3.246 | 1.00 | 58.18 | B |
| 4993 | O | ASP | B | 1168 | 22.155 | 15.653 | −3.094 | 1.00 | 59.93 | B |
| 4994 | N | VAL | B | 1169 | 24.016 | 14.686 | −2.353 | 1.00 | 56.75 | B |
| 4995 | CA | VAL | B | 1169 | 23.279 | 14.171 | −1.231 | 1.00 | 56.52 | B |
| 4996 | CB | VAL | B | 1169 | 22.566 | 12.822 | −1.494 | 1.00 | 54.41 | B |
| 4997 | CG1 | VAL | B | 1169 | 23.514 | 11.644 | −1.728 | 1.00 | 56.66 | B |
| 4998 | CG2 | VAL | B | 1169 | 21.772 | 12.588 | −0.360 | 1.00 | 58.02 | B |
| 4999 | C | VAL | B | 1169 | 24.153 | 14.243 | −0.082 | 1.00 | 54.47 | B |
| 5000 | O | VAL | B | 1169 | 25.391 | 14.271 | −0.320 | 1.00 | 57.01 | B |
| 5001 | N | PRO | B | 1170 | 23.570 | 14.394 | 1.127 | 1.00 | 52.30 | B |
| 5002 | CD | PRO | B | 1170 | 22.124 | 14.347 | 1.461 | 1.00 | 50.51 | B |
| 5003 | CA | PRO | B | 1170 | 24.384 | 14.657 | 2.325 | 1.00 | 49.97 | B |
| 5004 | CB | PRO | B | 1170 | 23.358 | 15.364 | 3.315 | 1.00 | 49.10 | B |
| 5005 | CG | PRO | B | 1170 | 22.078 | 14.732 | 3.009 | 1.00 | 44.98 | B |
| 5006 | C | PRO | B | 1170 | 24.573 | 13.250 | 2.808 | 1.00 | 49.59 | B |
| 5007 | O | PRO | B | 1170 | 23.820 | 12.780 | 3.543 | 1.00 | 49.70 | B |
| 5008 | N | TRP | B | 1171 | 25.577 | 12.545 | 2.399 | 1.00 | 50.44 | B |
| 5009 | CA | TRP | B | 1171 | 25.784 | 11.272 | 2.977 | 1.00 | 49.04 | B |
| 5010 | CB | TRP | B | 1171 | 26.039 | 10.360 | 1.735 | 1.00 | 48.41 | B |
| 5011 | CG | TRP | B | 1171 | 25.307 | 9.080 | 1.849 | 1.00 | 45.07 | B |
| 5012 | CD2 | TRP | B | 1171 | 23.878 | 8.967 | 1.801 | 1.00 | 37.88 | B |
| 5013 | CE2 | TRP | B | 1171 | 23.561 | 7.566 | 1.961 | 1.00 | 41.15 | B |
| 5014 | CE3 | TRP | B | 1171 | 22.842 | 9.912 | 1.583 | 1.00 | 34.77 | B |
| 5015 | CD1 | TRP | B | 1171 | 25.839 | 7.768 | 2.041 | 1.00 | 38.99 | B |
| 5016 | NE1 | TRP | B | 1171 | 24.768 | 6.883 | 2.133 | 1.00 | 48.14 | B |
| 5017 | CZ2 | TRP | B | 1171 | 22.252 | 7.076 | 1.900 | 1.00 | 36.51 | B |
| 5018 | CZ3 | TRP | B | 1171 | 21.495 | 9.484 | 1.618 | 1.00 | 36.51 | B |
| 5019 | CH2 | TRP | B | 1171 | 21.212 | 8.018 | 1.792 | 1.00 | 42.35 | B |
| 5020 | C | TRP | B | 1171 | 27.019 | 11.220 | 3.898 | 1.00 | 47.87 | B |
| 5021 | O | TRP | B | 1171 | 27.987 | 10.828 | 3.403 | 1.00 | 48.58 | B |
| 5022 | N | GLY | B | 1172 | 26.973 | 11.560 | 5.195 | 1.00 | 47.59 | B |
| 5023 | CA | GLY | B | 1172 | 27.993 | 11.137 | 6.178 | 1.00 | 45.94 | B |
| 5024 | C | GLY | B | 1172 | 28.141 | 12.049 | 7.409 | 1.00 | 47.54 | B |
| 5025 | O | GLY | B | 1172 | 27.163 | 12.841 | 7.764 | 1.00 | 46.74 | B |
| 5026 | N | VAL | B | 1173 | 29.314 | 11.964 | 8.102 | 1.00 | 44.61 | B |
| 5027 | CA | VAL | B | 1173 | 29.573 | 12.941 | 9.077 | 1.00 | 46.16 | B |
| 5028 | CB | VAL | B | 1173 | 30.843 | 12.713 | 10.062 | 1.00 | 47.94 | B |
| 5029 | CG1 | VAL | B | 1173 | 30.426 | 12.751 | 11.604 | 1.00 | 46.53 | B |
| 5030 | CG2 | VAL | B | 1173 | 32.008 | 11.693 | 9.644 | 1.00 | 47.64 | B |
| 5031 | C | VAL | B | 1173 | 29.603 | 14.427 | 8.482 | 1.00 | 49.13 | B |
| 5032 | O | VAL | B | 1173 | 29.069 | 15.405 | 9.116 | 1.00 | 47.22 | B |
| 5033 | N | ASP | B | 1174 | 30.334 | 14.652 | 7.373 | 1.00 | 50.05 | B |
| 5034 | CA | ASP | B | 1174 | 30.081 | 15.881 | 6.635 | 1.00 | 51.49 | B |
| 5035 | CB | ASP | B | 1174 | 30.439 | 15.645 | 5.228 | 1.00 | 49.80 | B |
| 5036 | CG | ASP | B | 1174 | 31.428 | 14.617 | 5.136 | 1.00 | 51.90 | B |
| 5037 | OD1 | ASP | B | 1174 | 32.532 | 14.859 | 5.729 | 1.00 | 48.53 | B |
| 5038 | OD2 | ASP | B | 1174 | 31.097 | 13.589 | 4.469 | 1.00 | 50.54 | B |
| 5039 | C | ASP | B | 1174 | 28.630 | 16.428 | 6.539 | 1.00 | 52.61 | B |
| 5040 | O | ASP | B | 1174 | 28.349 | 17.167 | 5.579 | 1.00 | 51.92 | B |
| 5041 | N | SER | B | 1175 | 27.700 | 16.179 | 7.443 | 1.00 | 51.51 | B |
| 5042 | CA | SER | B | 1175 | 26.586 | 17.000 | 7.159 | 1.00 | 51.56 | B |
| 5043 | CB | SER | B | 1175 | 25.575 | 16.265 | 6.242 | 1.00 | 53.00 | B |
| 5044 | OG | SER | B | 1175 | 26.336 | 15.790 | 5.013 | 1.00 | 51.34 | B |
| 5045 | C | SER | B | 1175 | 26.126 | 17.548 | 8.427 | 1.00 | 52.58 | B |
| 5046 | O | SER | B | 1175 | 25.706 | 18.767 | 8.616 | 1.00 | 51.67 | B |
| 5047 | N | LEU | B | 1176 | 26.242 | 16.672 | 9.400 | 1.00 | 54.73 | B |
| 5048 | CA | LEU | B | 1176 | 25.668 | 17.089 | 10.724 | 1.00 | 54.53 | B |
| 5049 | CB | LEU | B | 1176 | 25.780 | 16.037 | 11.721 | 1.00 | 52.36 | B |
| 5050 | CG | LEU | B | 1176 | 25.066 | 16.421 | 12.958 | 1.00 | 52.29 | B |
| 5051 | CD1 | LEU | B | 1176 | 23.599 | 16.763 | 12.729 | 1.00 | 53.20 | B |
| 5052 | CD2 | LEU | B | 1176 | 25.207 | 15.380 | 14.016 | 1.00 | 55.15 | B |
| 5053 | C | LEU | B | 1176 | 26.315 | 18.438 | 11.098 | 1.00 | 56.89 | B |
| 5054 | O | LEU | B | 1176 | 27.571 | 18.529 | 10.910 | 1.00 | 55.00 | B |
| 5055 | N | ILE | B | 1177 | 25.447 | 19.482 | 11.342 | 1.00 | 58.02 | B |
| 5056 | CA | ILE | B | 1177 | 25.852 | 20.735 | 11.838 | 1.00 | 60.78 | B |
| 5057 | CB | ILE | B | 1177 | 25.486 | 21.882 | 10.945 | 1.00 | 62.68 | B |
| 5058 | CG2 | ILE | B | 1177 | 24.964 | 23.262 | 11.816 | 1.00 | 60.44 | B |
| 5059 | CG1 | ILE | B | 1177 | 26.683 | 22.246 | 10.106 | 1.00 | 64.46 | B |
| 5060 | CD1 | ILE | B | 1177 | 27.887 | 22.598 | 10.951 | 1.00 | 60.83 | B |
| 5061 | C | ILE | B | 1177 | 25.032 | 21.071 | 12.989 | 1.00 | 64.17 | B |
| 5062 | O | ILE | B | 1177 | 23.747 | 20.894 | 12.930 | 1.00 | 61.74 | B |
| 5063 | N | THR | B | 1178 | 25.739 | 21.695 | 13.973 | 1.00 | 65.74 | B |
| 5064 | CA | THR | B | 1178 | 25.044 | 22.042 | 15.248 | 1.00 | 68.16 | B |
| 5065 | CB | THR | B | 1178 | 25.580 | 21.160 | 16.445 | 1.00 | 69.28 | B |
| 5066 | OG1 | THR | B | 1178 | 26.195 | 19.907 | 15.948 | 1.00 | 73.30 | B |
| 5067 | CG2 | THR | B | 1178 | 24.393 | 20.807 | 17.435 | 1.00 | 67.53 | B |
| 5068 | C | THR | B | 1178 | 24.795 | 23.554 | 15.579 | 1.00 | 67.67 | B |
| 5069 | O | THR | B | 1178 | 25.609 | 24.344 | 15.355 | 1.00 | 69.24 | B |
| 5070 | N | LEU | B | 1179 | 23.627 | 23.938 | 16.069 | 1.00 | 67.37 | B |
| 5071 | CA | LEU | B | 1179 | 23.142 | 25.342 | 16.210 | 1.00 | 66.19 | B |
| 5072 | CB | LEU | B | 1179 | 21.778 | 25.641 | 15.432 | 1.00 | 64.43 | B |
| 5073 | CG | LEU | B | 1179 | 21.685 | 25.636 | 13.901 | 1.00 | 63.78 | B |
| 5074 | CD1 | LEU | B | 1179 | 20.419 | 26.249 | 13.492 | 1.00 | 68.71 | B |
| 5075 | CD2 | LEU | B | 1179 | 22.855 | 26.371 | 13.160 | 1.00 | 59.53 | B |
| 5076 | C | LEU | B | 1179 | 22.771 | 25.434 | 17.651 | 1.00 | 65.69 | B |
| 5077 | O | LEU | B | 1179 | 21.593 | 25.263 | 17.971 | 1.00 | 61.26 | B |
| 5078 | N | ALA | B | 1180 | 23.730 | 25.651 | 18.553 | 1.00 | 66.92 | B |
| 5079 | CA | ALA | B | 1180 | 23.229 | 25.543 | 19.929 | 1.00 | 68.83 | B |
| 5080 | CB | ALA | B | 1180 | 23.857 | 24.310 | 20.728 | 1.00 | 68.51 | B |
| 5081 | C | ALA | B | 1180 | 23.351 | 26.934 | 20.578 | 1.00 | 68.71 | B |
| 5082 | O | ALA | B | 1180 | 23.806 | 27.865 | 19.855 | 1.00 | 69.04 | B |
| 5083 | N | PHE | B | 1181 | 22.882 | 27.125 | 21.828 | 1.00 | 67.23 | B |
| 5084 | CA | PHE | B | 1181 | 22.747 | 28.493 | 22.325 | 1.00 | 66.78 | B |
| 5085 | CB | PHE | B | 1181 | 22.190 | 28.479 | 23.676 | 1.00 | 65.44 | B |
| 5086 | CG | PHE | B | 1181 | 20.822 | 27.829 | 23.707 | 1.00 | 67.32 | B |
| 5087 | CD1 | PHE | B | 1181 | 19.915 | 28.056 | 22.682 | 1.00 | 63.04 | B |
| 5088 | CD2 | PHE | B | 1181 | 20.445 | 27.026 | 24.720 | 1.00 | 64.17 | B |
| 5089 | CE1 | PHE | B | 1181 | 18.694 | 27.546 | 22.695 | 1.00 | 60.93 | B |
| 5090 | CE2 | PHE | B | 1181 | 19.232 | 26.554 | 24.721 | 1.00 | 68.19 | B |
| 5091 | CZ | PHE | B | 1181 | 18.319 | 26.822 | 23.702 | 1.00 | 64.91 | B |
| 5092 | C | PHE | B | 1181 | 24.062 | 29.189 | 22.279 | 1.00 | 68.44 | B |
| 5093 | O | PHE | B | 1181 | 25.135 | 28.561 | 22.538 | 1.00 | 68.27 | B |
| 5094 | N | GLN | B | 1182 | 24.060 | 30.437 | 21.797 | 1.00 | 68.03 | B |
| 5095 | CA | GLN | B | 1182 | 25.204 | 31.235 | 22.179 | 1.00 | 66.43 | B |
| 5096 | CB | GLN | B | 1182 | 25.714 | 32.012 | 20.978 | 1.00 | 64.98 | B |
| 5097 | CG | GLN | B | 1182 | 26.814 | 31.261 | 20.321 | 1.00 | 69.22 | B |
| 5098 | CD | GLN | B | 1182 | 28.119 | 32.105 | 20.015 | 1.00 | 73.97 | B |
| 5099 | OE1 | GLN | B | 1182 | 28.229 | 33.292 | 20.382 | 1.00 | 77.27 | B |
| 5100 | NE2 | GLN | B | 1182 | 29.102 | 31.474 | 19.355 | 1.00 | 67.36 | B |
| 5101 | C | GLN | B | 1182 | 24.987 | 32.120 | 23.470 | 1.00 | 66.45 | B |
| 5102 | O | GLN | B | 1182 | 24.486 | 31.736 | 24.612 | 1.00 | 63.70 | B |
| 5103 | N | ASP | B | 1183 | 25.370 | 33.356 | 23.256 | 1.00 | 67.03 | B |
| 5104 | CA | ASP | B | 1183 | 25.647 | 34.194 | 24.368 | 1.00 | 69.64 | B |
| 5105 | CB | ASP | B | 1183 | 26.887 | 35.099 | 24.110 | 1.00 | 67.88 | B |
| 5106 | CG | ASP | B | 1183 | 27.311 | 35.116 | 22.591 | 1.00 | 66.51 | B |
| 5107 | OD1 | ASP | B | 1183 | 26.406 | 35.162 | 21.604 | 1.00 | 51.11 | B |
| 5108 | OD2 | ASP | B | 1183 | 28.562 | 35.023 | 22.476 | 1.00 | 56.49 | B |
| 5109 | C | ASP | B | 1183 | 24.351 | 34.911 | 24.419 | 1.00 | 71.57 | B |
| 5110 | O | ASP | B | 1183 | 24.303 | 36.095 | 24.640 | 1.00 | 72.77 | B |
| 5111 | N | GLN | B | 1184 | 23.283 | 34.153 | 24.175 | 1.00 | 73.81 | B |
| 5112 | CA | GLN | B | 1184 | 22.116 | 34.757 | 23.463 | 1.00 | 74.78 | B |
| 5113 | CB | GLN | B | 1184 | 21.832 | 36.179 | 24.033 | 1.00 | 72.28 | B |
| 5114 | CG | GLN | B | 1184 | 21.273 | 36.181 | 25.546 | 1.00 | 72.20 | B |
| 5115 | CD | GLN | B | 1184 | 19.730 | 35.650 | 25.748 | 1.00 | 76.24 | B |
| 5116 | OE1 | GLN | B | 1184 | 18.778 | 36.465 | 26.072 | 1.00 | 69.72 | B |
| 5117 | NE2 | GLN | B | 1184 | 19.520 | 34.274 | 25.582 | 1.00 | 74.79 | B |
| 5118 | C | GLN | B | 1184 | 22.010 | 34.714 | 21.866 | 1.00 | 73.84 | B |
| 5119 | O | GLN | B | 1184 | 20.977 | 35.015 | 21.394 | 1.00 | 75.38 | B |
| 5120 | N | ARG | B | 1185 | 23.038 | 34.410 | 21.070 | 1.00 | 73.99 | B |
| 5121 | CA | ARG | B | 1185 | 22.920 | 34.562 | 19.557 | 1.00 | 75.06 | B |
| 5122 | CB | ARG | B | 1185 | 24.200 | 34.994 | 18.854 | 1.00 | 74.71 | B |
| 5123 | CG | ARG | B | 1185 | 24.497 | 36.461 | 18.786 | 1.00 | 73.16 | B |
| 5124 | CD | ARG | B | 1185 | 25.195 | 36.534 | 17.453 | 1.00 | 77.00 | B |
| 5125 | NE | ARG | B | 1185 | 24.999 | 37.821 | 16.696 | 1.00 | 87.06 | B |
| 5126 | CZ | ARG | B | 1185 | 24.173 | 38.124 | 15.661 | 1.00 | 82.00 | B |
| 5127 | NH1 | ARG | B | 1185 | 23.331 | 37.286 | 15.089 | 1.00 | 77.45 | B |
| 5128 | NH2 | ARG | B | 1185 | 24.202 | 39.352 | 15.184 | 1.00 | 85.40 | B |
| 5129 | C | ARG | B | 1185 | 22.484 | 33.267 | 18.856 | 1.00 | 75.69 | B |
| 5130 | O | ARG | B | 1185 | 21.276 | 32.986 | 18.768 | 1.00 | 77.37 | B |
| 5131 | N | TYR | B | 1186 | 23.453 | 32.496 | 18.345 | 1.00 | 74.20 | B |
| 5132 | CA | TYR | B | 1186 | 23.200 | 31.080 | 17.965 | 1.00 | 72.40 | B |
| 5133 | CB | TYR | B | 1186 | 21.781 | 30.973 | 17.342 | 1.00 | 71.03 | B |
| 5134 | CG | TYR | B | 1186 | 20.742 | 30.468 | 18.233 | 1.00 | 62.98 | B |
| 5135 | CD1 | TYR | B | 1186 | 20.723 | 29.151 | 18.606 | 1.00 | 58.13 | B |
| 5136 | CE1 | TYR | B | 1186 | 19.752 | 28.705 | 19.514 | 1.00 | 63.92 | B |
| 5137 | CD2 | TYR | B | 1186 | 19.815 | 31.330 | 18.738 | 1.00 | 55.24 | B |
| 5138 | CE2 | TYR | B | 1186 | 18.873 | 30.944 | 19.612 | 1.00 | 55.26 | B |
| 5139 | CZ | TYR | B | 1186 | 18.781 | 29.608 | 20.001 | 1.00 | 62.64 | B |
| 5140 | OH | TYR | B | 1186 | 17.743 | 29.181 | 20.847 | 1.00 | 60.02 | B |
| 5141 | C | TYR | B | 1186 | 24.220 | 30.448 | 16.940 | 1.00 | 72.33 | B |
| 5142 | O | TYR | B | 1186 | 23.898 | 30.257 | 15.731 | 1.00 | 72.81 | B |
| 5143 | N | SER | B | 1187 | 25.419 | 30.130 | 17.418 | 1.00 | 71.14 | B |
| 5144 | CA | SER | B | 1187 | 26.456 | 29.566 | 16.562 | 1.00 | 70.26 | B |
| 5145 | CB | SER | B | 1187 | 27.665 | 29.349 | 17.403 | 1.00 | 70.80 | B |
| 5146 | OG | SER | B | 1187 | 27.758 | 28.054 | 17.913 | 1.00 | 65.93 | B |
| 5147 | C | SER | B | 1187 | 26.164 | 28.245 | 15.743 | 1.00 | 70.53 | B |
| 5148 | O | SER | B | 1187 | 24.990 | 27.717 | 15.665 | 1.00 | 70.23 | B |
| 5149 | N | VAL | B | 1188 | 27.271 | 27.724 | 15.155 | 1.00 | 67.55 | B |
| 5150 | CA | VAL | B | 1188 | 27.182 | 26.682 | 14.138 | 1.00 | 61.21 | B |
| 5151 | CB | VAL | B | 1188 | 27.024 | 27.243 | 12.778 | 1.00 | 57.23 | B |
| 5152 | CG1 | VAL | B | 1188 | 25.974 | 28.149 | 12.908 | 1.00 | 53.10 | B |
| 5153 | CG2 | VAL | B | 1188 | 28.264 | 27.922 | 12.402 | 1.00 | 54.13 | B |
| 5154 | C | VAL | B | 1188 | 28.353 | 25.777 | 14.207 | 1.00 | 61.91 | B |
| 5155 | O | VAL | B | 1188 | 29.285 | 25.896 | 13.405 | 1.00 | 64.50 | B |
| 5156 | N | GLN | B | 1189 | 28.263 | 24.832 | 15.108 | 1.00 | 61.15 | B |
| 5157 | CA | GLN | B | 1189 | 29.342 | 23.879 | 15.420 | 1.00 | 63.51 | B |
| 5158 | CB | GLN | B | 1189 | 29.061 | 23.257 | 16.808 | 1.00 | 62.02 | B |
| 5159 | CG | GLN | B | 1189 | 29.730 | 21.959 | 17.021 | 1.00 | 60.53 | B |
| 5160 | CD | GLN | B | 1189 | 29.211 | 21.275 | 18.274 | 1.00 | 68.31 | B |
| 5161 | OE1 | GLN | B | 1189 | 29.166 | 21.883 | 19.396 | 1.00 | 70.12 | B |
| 5162 | NE2 | GLN | B | 1189 | 28.786 | 19.990 | 18.108 | 1.00 | 67.19 | B |
| 5163 | C | GLN | B | 1189 | 29.601 | 22.807 | 14.308 | 1.00 | 64.43 | B |
| 5164 | O | GLN | B | 1189 | 28.825 | 21.856 | 14.137 | 1.00 | 67.10 | B |
| 5165 | N | THR | B | 1190 | 30.631 | 22.962 | 13.527 | 1.00 | 65.33 | B |
| 5166 | CA | THR | B | 1190 | 31.029 | 21.926 | 12.544 | 1.00 | 69.17 | B |
| 5167 | CB | THR | B | 1190 | 32.519 | 22.158 | 12.284 | 1.00 | 70.80 | B |
| 5168 | OG1 | THR | B | 1190 | 33.152 | 22.571 | 13.536 | 1.00 | 76.36 | B |
| 5169 | CG2 | THR | B | 1190 | 32.711 | 23.221 | 11.276 | 1.00 | 69.09 | B |
| 5170 | C | THR | B | 1190 | 31.011 | 20.406 | 12.980 | 1.00 | 69.88 | B |
| 5171 | O | THR | B | 1190 | 30.013 | 19.899 | 13.570 | 1.00 | 70.24 | B |
| 5172 | N | ALA | B | 1191 | 32.123 | 19.654 | 12.762 | 1.00 | 67.79 | B |
| 5173 | CA | ALA | B | 1191 | 31.954 | 18.197 | 13.001 | 1.00 | 67.16 | B |
| 5174 | CB | ALA | B | 1191 | 32.746 | 17.373 | 11.919 | 1.00 | 66.67 | B |
| 5175 | C | ALA | B | 1191 | 32.276 | 17.642 | 14.434 | 1.00 | 65.02 | B |
| 5176 | O | ALA | B | 1191 | 31.916 | 16.529 | 14.789 | 1.00 | 62.69 | B |
| 5177 | N | ASP | B | 1192 | 32.889 | 18.487 | 15.231 | 1.00 | 63.61 | B |
| 5178 | CA | ASP | B | 1192 | 34.085 | 18.149 | 15.934 | 1.00 | 61.13 | B |
| 5179 | CB | ASP | B | 1192 | 35.262 | 18.147 | 14.908 | 1.00 | 61.71 | B |
| 5180 | CG | ASP | B | 1192 | 35.511 | 19.592 | 14.135 | 1.00 | 56.03 | B |
| 5181 | OD1 | ASP | B | 1192 | 34.859 | 20.617 | 14.397 | 1.00 | 55.47 | B |
| 5182 | OD2 | ASP | B | 1192 | 36.425 | 19.691 | 13.269 | 1.00 | 43.37 | B |
| 5183 | C | ASP | B | 1192 | 34.193 | 19.370 | 16.753 | 1.00 | 60.78 | B |
| 5184 | O | ASP | B | 1192 | 35.278 | 19.855 | 16.961 | 1.00 | 61.28 | B |
| 5185 | N | HIS | B | 1193 | 33.045 | 19.972 | 17.004 | 1.00 | 60.11 | B |
| 5186 | CA | HIS | B | 1193 | 32.835 | 20.934 | 18.084 | 1.00 | 60.11 | B |
| 5187 | CB | HIS | B | 1193 | 33.566 | 20.455 | 19.304 | 1.00 | 58.79 | B |
| 5188 | CG | HIS | B | 1193 | 32.957 | 19.210 | 19.900 | 1.00 | 60.16 | B |
| 5189 | CD2 | HIS | B | 1193 | 31.790 | 19.027 | 20.602 | 1.00 | 58.77 | B |
| 5190 | ND1 | HIS | B | 1193 | 33.521 | 17.949 | 19.765 | 1.00 | 57.05 | B |
| 5191 | CE1 | HIS | B | 1193 | 32.737 | 17.048 | 20.375 | 1.00 | 57.17 | B |
| 5192 | NE2 | HIS | B | 1193 | 31.686 | 17.669 | 20.894 | 1.00 | 52.86 | B |
| 5193 | C | HIS | B | 1193 | 33.042 | 22.430 | 17.746 | 1.00 | 60.17 | B |
| 5194 | O | HIS | B | 1193 | 32.319 | 23.305 | 18.234 | 1.00 | 59.66 | B |
| 5195 | N | ARG | B | 1194 | 33.951 | 22.685 | 16.818 | 1.00 | 60.63 | B |
| 5196 | CA | ARG | B | 1194 | 34.419 | 24.057 | 16.554 | 1.00 | 61.63 | B |
| 5197 | CB | ARG | B | 1194 | 35.920 | 24.120 | 15.983 | 1.00 | 60.52 | B |
| 5198 | CG | ARG | B | 1194 | 36.268 | 22.945 | 15.086 | 1.00 | 57.21 | B |
| 5199 | CD | ARG | B | 1194 | 37.659 | 23.135 | 14.237 | 1.00 | 64.97 | B |
| 5200 | NE | ARG | B | 1194 | 37.564 | 22.700 | 12.753 | 1.00 | 64.47 | B |
| 5201 | CZ | ARG | B | 1194 | 38.326 | 23.098 | 11.717 | 1.00 | 53.20 | B |
| 5202 | NH1 | ARG | B | 1194 | 39.339 | 23.952 | 11.822 | 1.00 | 51.70 | B |
| 5203 | NH2 | ARG | B | 1194 | 38.052 | 22.624 | 10.548 | 1.00 | 50.09 | B |
| 5204 | C | ARG | B | 1194 | 33.341 | 24.830 | 15.721 | 1.00 | 61.10 | B |
| 5205 | O | ARG | B | 1194 | 32.554 | 24.210 | 14.949 | 1.00 | 62.40 | B |
| 5206 | N | PHE | B | 1195 | 33.399 | 26.143 | 15.836 | 1.00 | 59.05 | B |
| 5207 | CA | PHE | B | 1195 | 32.315 | 26.955 | 15.629 | 1.00 | 58.39 | B |
| 5208 | CB | PHE | B | 1195 | 32.200 | 27.675 | 16.882 | 1.00 | 58.85 | B |
| 5209 | CG | PHE | B | 1195 | 32.126 | 26.789 | 18.152 | 1.00 | 61.76 | B |
| 5210 | CD1 | PHE | B | 1195 | 33.260 | 26.541 | 18.927 | 1.00 | 62.70 | B |
| 5211 | CD2 | PHE | B | 1195 | 30.870 | 26.398 | 18.701 | 1.00 | 64.66 | B |
| 5212 | CE1 | PHE | B | 1195 | 33.161 | 25.822 | 20.191 | 1.00 | 65.05 | B |
| 5213 | CE2 | PHE | B | 1195 | 30.774 | 25.658 | 19.936 | 1.00 | 65.10 | B |
| 5214 | CZ | PHE | B | 1195 | 31.912 | 25.373 | 20.681 | 1.00 | 61.99 | B |
| 5215 | C | PHE | B | 1195 | 32.679 | 27.940 | 14.579 | 1.00 | 59.02 | B |
| 5216 | O | PHE | B | 1195 | 33.800 | 28.463 | 14.576 | 1.00 | 59.18 | B |
| 5217 | N | LEU | B | 1196 | 31.783 | 28.207 | 13.620 | 1.00 | 61.21 | B |
| 5218 | CA | LEU | B | 1196 | 31.964 | 29.399 | 12.647 | 1.00 | 60.50 | B |
| 5219 | CB | LEU | B | 1196 | 30.808 | 29.452 | 11.648 | 1.00 | 60.63 | B |
| 5220 | CG | LEU | B | 1196 | 31.322 | 29.810 | 10.234 | 1.00 | 60.41 | B |
| 5221 | CD1 | LEU | B | 1196 | 32.314 | 28.644 | 9.857 | 1.00 | 54.21 | B |
| 5222 | CD2 | LEU | B | 1196 | 29.952 | 29.697 | 9.526 | 1.00 | 60.92 | B |
| 5223 | C | LEU | B | 1196 | 32.025 | 30.853 | 13.202 | 1.00 | 59.80 | B |
| 5224 | O | LEU | B | 1196 | 31.139 | 31.280 | 13.914 | 1.00 | 59.04 | B |
| 5225 | N | ARG | B | 1197 | 33.034 | 31.627 | 12.835 | 1.00 | 61.17 | B |
| 5226 | CA | ARG | B | 1197 | 32.981 | 33.068 | 13.065 | 1.00 | 61.23 | B |
| 5227 | CB | ARG | B | 1197 | 34.395 | 33.639 | 13.452 | 1.00 | 63.28 | B |
| 5228 | CG | ARG | B | 1197 | 34.560 | 35.289 | 13.297 | 1.00 | 62.64 | B |
| 5229 | CD | ARG | B | 1197 | 36.025 | 35.736 | 13.057 | 1.00 | 63.62 | B |
| 5230 | NE | ARG | B | 1197 | 36.944 | 34.932 | 13.855 | 1.00 | 67.96 | B |
| 5231 | CZ | ARG | B | 1197 | 37.937 | 34.225 | 13.334 | 1.00 | 62.03 | B |
| 5232 | NH1 | ARG | B | 1197 | 38.137 | 34.335 | 12.050 | 1.00 | 59.59 | B |
| 5233 | NH2 | ARG | B | 1197 | 38.743 | 33.493 | 14.107 | 1.00 | 54.12 | B |
| 5234 | C | ARG | B | 1197 | 32.733 | 33.505 | 11.708 | 1.00 | 60.28 | B |
| 5235 | O | ARG | B | 1197 | 33.278 | 32.895 | 10.758 | 1.00 | 59.90 | B |
| 5236 | N | HIS | B | 1198 | 32.020 | 34.590 | 11.592 | 1.00 | 62.22 | B |
| 5237 | CA | HIS | B | 1198 | 31.636 | 35.137 | 10.241 | 1.00 | 66.67 | B |
| 5238 | CB | HIS | B | 1198 | 30.783 | 36.382 | 10.391 | 1.00 | 64.94 | B |
| 5239 | CG | HIS | B | 1198 | 31.271 | 37.322 | 11.462 | 1.00 | 72.94 | B |
| 5240 | CD2 | HIS | B | 1198 | 32.147 | 38.363 | 11.400 | 1.00 | 77.15 | B |
| 5241 | ND1 | HIS | B | 1198 | 30.802 | 37.305 | 12.767 | 1.00 | 74.29 | B |
| 5242 | CE1 | HIS | B | 1198 | 31.354 | 38.304 | 13.443 | 1.00 | 76.99 | B |
| 5243 | NE2 | HIS | B | 1198 | 32.128 | 38.993 | 12.622 | 1.00 | 76.12 | B |
| 5244 | C | HIS | B | 1198 | 32.773 | 35.402 | 9.262 | 1.00 | 68.24 | B |
| 5245 | O | HIS | B | 1198 | 32.954 | 36.571 | 8.723 | 1.00 | 71.98 | B |
| 5246 | N | ASP | B | 1199 | 33.600 | 34.371 | 9.043 | 1.00 | 69.42 | B |
| 5247 | CA | ASP | B | 1199 | 34.863 | 34.603 | 8.313 | 1.00 | 67.97 | B |
| 5248 | CB | ASP | B | 1199 | 35.985 | 35.185 | 9.228 | 1.00 | 66.27 | B |
| 5249 | CG | ASP | B | 1199 | 37.137 | 34.234 | 9.413 | 1.00 | 64.18 | B |
| 5250 | OD1 | ASP | B | 1199 | 38.347 | 34.621 | 9.509 | 1.00 | 50.97 | B |
| 5251 | OD2 | ASP | B | 1199 | 36.769 | 33.047 | 9.464 | 1.00 | 66.34 | B |
| 5252 | C | ASP | B | 1199 | 35.357 | 33.457 | 7.348 | 1.00 | 68.77 | B |
| 5253 | O | ASP | B | 1199 | 36.512 | 33.648 | 6.745 | 1.00 | 65.84 | B |
| 5254 | N | GLY | B | 1200 | 34.490 | 32.393 | 7.146 | 1.00 | 67.85 | B |
| 5255 | CA | GLY | B | 1200 | 34.896 | 31.013 | 6.557 | 1.00 | 68.84 | B |
| 5256 | C | GLY | B | 1200 | 35.743 | 29.925 | 7.357 | 1.00 | 68.75 | B |
| 5257 | O | GLY | B | 1200 | 36.379 | 28.944 | 6.843 | 1.00 | 66.66 | B |
| 5258 | N | ARG | B | 1201 | 35.713 | 30.105 | 8.662 | 1.00 | 68.93 | B |
| 5259 | CA | ARG | B | 1201 | 36.717 | 29.518 | 9.492 | 1.00 | 67.55 | B |
| 5260 | CB | ARG | B | 1201 | 37.803 | 30.590 | 9.860 | 1.00 | 69.26 | B |
| 5261 | CG | ARG | B | 1201 | 39.069 | 30.728 | 8.756 | 1.00 | 71.25 | B |
| 5262 | CD | ARG | B | 1201 | 40.565 | 30.335 | 9.403 | 1.00 | 67.30 | B |
| 5263 | NE | ARG | B | 1201 | 40.389 | 30.153 | 10.817 | 1.00 | 67.36 | B |
| 5264 | CZ | ARG | B | 1201 | 39.743 | 29.118 | 11.384 | 1.00 | 68.68 | B |
| 5265 | NH1 | ARG | B | 1201 | 39.577 | 29.109 | 12.727 | 1.00 | 65.40 | B |
| 5266 | NH2 | ARG | B | 1201 | 39.273 | 28.087 | 10.616 | 1.00 | 62.72 | B |
| 5267 | C | ARG | B | 1201 | 36.137 | 28.749 | 10.692 | 1.00 | 65.68 | B |
| 5268 | O | ARG | B | 1201 | 34.895 | 28.803 | 11.061 | 1.00 | 64.37 | B |
| 5269 | N | LEU | B | 1202 | 37.043 | 27.931 | 11.238 | 1.00 | 64.35 | B |
| 5270 | CA | LEU | B | 1202 | 36.704 | 27.095 | 12.408 | 1.00 | 60.92 | B |
| 5271 | CB | LEU | B | 1202 | 36.383 | 25.666 | 11.921 | 1.00 | 59.77 | B |
| 5272 | CG | LEU | B | 1202 | 34.973 | 24.990 | 11.804 | 1.00 | 55.47 | B |
| 5273 | CD1 | LEU | B | 1202 | 35.191 | 23.537 | 12.544 | 1.00 | 45.59 | B |
| 5274 | CD2 | LEU | B | 1202 | 33.722 | 25.784 | 12.398 | 1.00 | 43.52 | B |
| 5275 | C | LEU | B | 1202 | 37.736 | 27.296 | 13.559 | 1.00 | 59.15 | B |
| 5276 | O | LEU | B | 1202 | 38.915 | 27.075 | 13.467 | 1.00 | 54.93 | B |
| 5277 | N | VAL | B | 1203 | 37.273 | 27.928 | 14.607 | 1.00 | 62.39 | B |
| 5278 | CA | VAL | B | 1203 | 38.195 | 28.294 | 15.797 | 1.00 | 63.20 | B |
| 5279 | CB | VAL | B | 1203 | 37.666 | 29.381 | 16.732 | 1.00 | 61.66 | B |
| 5280 | CG1 | VAL | B | 1203 | 37.295 | 30.674 | 15.982 | 1.00 | 63.82 | B |
| 5281 | CG2 | VAL | B | 1203 | 36.461 | 28.898 | 17.501 | 1.00 | 58.28 | B |
| 5282 | C | VAL | B | 1203 | 38.263 | 27.087 | 16.708 | 1.00 | 65.37 | B |
| 5283 | O | VAL | B | 1203 | 38.916 | 26.037 | 16.313 | 1.00 | 67.18 | B |
| 5284 | N | ALA | B | 1204 | 37.549 | 27.213 | 17.860 | 1.00 | 64.73 | B |
| 5285 | CA | ALA | B | 1204 | 37.881 | 26.462 | 19.116 | 1.00 | 65.60 | B |
| 5286 | CB | ALA | B | 1204 | 38.845 | 27.224 | 19.942 | 1.00 | 66.68 | B |
| 5287 | C | ALA | B | 1204 | 36.720 | 26.256 | 19.980 | 1.00 | 66.21 | B |
| 5288 | O | ALA | B | 1204 | 36.084 | 25.134 | 19.941 | 1.00 | 65.59 | B |
| 5289 | N | ARG | B | 1205 | 36.513 | 27.328 | 20.790 | 1.00 | 66.04 | B |
| 5290 | CA | ARG | B | 1205 | 35.477 | 27.515 | 21.879 | 1.00 | 65.19 | B |
| 5291 | CB | ARG | B | 1205 | 36.154 | 27.529 | 23.305 | 1.00 | 62.46 | B |
| 5292 | CG | ARG | B | 1205 | 37.417 | 28.294 | 23.253 | 1.00 | 62.21 | B |
| 5293 | CD | ARG | B | 1205 | 38.714 | 27.828 | 24.121 | 1.00 | 64.19 | B |
| 5294 | NE | ARG | B | 1205 | 39.919 | 28.227 | 23.373 | 1.00 | 60.95 | B |
| 5295 | CZ | ARG | B | 1205 | 40.017 | 29.347 | 22.626 | 1.00 | 69.25 | B |
| 5296 | NH1 | ARG | B | 1205 | 39.052 | 30.260 | 22.644 | 1.00 | 69.28 | B |
| 5297 | NH2 | ARG | B | 1205 | 41.124 | 29.634 | 21.903 | 1.00 | 75.42 | B |
| 5298 | C | ARG | B | 1205 | 34.795 | 28.877 | 21.456 | 1.00 | 65.35 | B |
| 5299 | O | ARG | B | 1205 | 35.433 | 29.742 | 20.825 | 1.00 | 65.26 | B |
| 5300 | N | PRO | B | 1206 | 33.528 | 29.121 | 21.808 | 1.00 | 66.34 | B |
| 5301 | CD | PRO | B | 1206 | 32.735 | 28.796 | 23.034 | 1.00 | 66.50 | B |
| 5302 | CA | PRO | B | 1206 | 32.866 | 30.150 | 20.872 | 1.00 | 65.88 | B |
| 5303 | CB | PRO | B | 1206 | 31.412 | 30.228 | 21.355 | 1.00 | 66.02 | B |
| 5304 | CG | PRO | B | 1206 | 31.243 | 29.396 | 22.707 | 1.00 | 64.96 | B |
| 5305 | C | PRO | B | 1206 | 33.532 | 31.532 | 21.101 | 1.00 | 66.60 | B |
| 5306 | O | PRO | B | 1206 | 34.734 | 31.559 | 21.387 | 1.00 | 67.12 | B |
| 5307 | N | GLU | B | 1207 | 32.797 | 32.636 | 21.032 | 1.00 | 65.27 | B |
| 5308 | CA | GLU | B | 1207 | 33.385 | 33.973 | 21.215 | 1.00 | 65.25 | B |
| 5309 | CB | GLU | B | 1207 | 34.761 | 34.147 | 20.489 | 1.00 | 63.95 | B |
| 5310 | CG | GLU | B | 1207 | 34.821 | 34.291 | 18.997 | 1.00 | 60.46 | B |
| 5311 | CD | GLU | B | 1207 | 35.684 | 33.200 | 18.506 | 1.00 | 56.03 | B |
| 5312 | OE1 | GLU | B | 1207 | 35.146 | 32.193 | 17.922 | 1.00 | 61.81 | B |
| 5313 | OE2 | GLU | B | 1207 | 36.881 | 33.227 | 18.885 | 1.00 | 52.58 | B |
| 5314 | C | GLU | B | 1207 | 32.396 | 35.239 | 21.051 | 1.00 | 65.05 | B |
| 5315 | O | GLU | B | 1207 | 31.175 | 35.105 | 21.286 | 1.00 | 66.30 | B |
| 5316 | N | PRO | B | 1208 | 32.894 | 36.447 | 20.692 | 1.00 | 63.15 | B |
| 5317 | CD | PRO | B | 1208 | 34.010 | 37.365 | 20.385 | 1.00 | 61.99 | B |
| 5318 | CA | PRO | B | 1208 | 31.653 | 37.069 | 20.459 | 1.00 | 63.47 | B |
| 5319 | CB | PRO | B | 1208 | 31.903 | 38.501 | 20.991 | 1.00 | 64.55 | B |
| 5320 | CG | PRO | B | 1208 | 33.618 | 38.591 | 21.128 | 1.00 | 61.21 | B |
| 5321 | C | PRO | B | 1208 | 31.459 | 36.943 | 18.893 | 1.00 | 64.00 | B |
| 5322 | O | PRO | B | 1208 | 30.336 | 36.637 | 18.507 | 1.00 | 61.98 | B |
| 5323 | N | ALA | B | 1209 | 32.566 | 37.088 | 18.088 | 1.00 | 64.00 | B |
| 5324 | CA | ALA | B | 1209 | 32.688 | 37.361 | 16.512 | 1.00 | 63.00 | B |
| 5325 | CB | ALA | B | 1209 | 33.925 | 38.177 | 16.196 | 1.00 | 60.78 | B |
| 5326 | C | ALA | B | 1209 | 32.700 | 36.062 | 15.653 | 1.00 | 62.87 | B |
| 5327 | O | ALA | B | 1209 | 33.651 | 35.782 | 14.953 | 1.00 | 60.14 | B |
| 5328 | N | THR | B | 1210 | 31.562 | 35.317 | 15.843 | 1.00 | 65.38 | B |
| 5329 | CA | THR | B | 1210 | 31.173 | 33.870 | 15.578 | 1.00 | 64.61 | B |
| 5330 | CB | THR | B | 1210 | 31.863 | 33.077 | 16.589 | 1.00 | 64.74 | B |
| 5331 | OG1 | THR | B | 1210 | 33.217 | 33.182 | 16.227 | 1.00 | 70.21 | B |
| 5332 | CG2 | THR | B | 1210 | 31.314 | 31.599 | 16.737 | 1.00 | 61.68 | B |
| 5333 | C | THR | B | 1210 | 29.630 | 33.409 | 15.599 | 1.00 | 64.46 | B |
| 5334 | O | THR | B | 1210 | 29.314 | 32.414 | 14.974 | 1.00 | 67.46 | B |
| 5335 | N | GLY | B | 1211 | 28.693 | 34.096 | 16.270 | 1.00 | 62.42 | B |
| 5336 | CA | GLY | B | 1211 | 27.254 | 33.615 | 16.401 | 1.00 | 62.02 | B |
| 5337 | C | GLY | B | 1211 | 25.974 | 34.315 | 15.819 | 1.00 | 60.76 | B |
| 5338 | O | GLY | B | 1211 | 25.977 | 35.531 | 15.574 | 1.00 | 60.18 | B |
| 5339 | N | TYR | B | 1212 | 24.853 | 33.590 | 15.668 | 1.00 | 60.27 | B |
| 5340 | CA | TYR | B | 1212 | 23.810 | 34.149 | 14.788 | 1.00 | 62.56 | B |
| 5341 | CB | TYR | B | 1212 | 23.792 | 33.524 | 13.426 | 1.00 | 60.37 | B |
| 5342 | CG | TYR | B | 1212 | 25.202 | 33.262 | 12.999 | 1.00 | 59.89 | B |
| 5343 | CD1 | TYR | B | 1212 | 25.955 | 34.299 | 12.395 | 1.00 | 53.50 | B |
| 5344 | CE1 | TYR | B | 1212 | 27.350 | 34.099 | 12.088 | 1.00 | 65.28 | B |
| 5345 | CD2 | TYR | B | 1212 | 25.816 | 31.959 | 13.237 | 1.00 | 58.01 | B |
| 5346 | CE2 | TYR | B | 1212 | 27.153 | 31.703 | 12.863 | 1.00 | 58.90 | B |
| 5347 | CZ | TYR | B | 1212 | 27.953 | 32.760 | 12.312 | 1.00 | 62.92 | B |
| 5348 | OH | TYR | B | 1212 | 29.274 | 32.486 | 11.928 | 1.00 | 53.54 | B |
| 5349 | C | TYR | B | 1212 | 22.396 | 34.257 | 15.272 | 1.00 | 64.57 | B |
| 5350 | O | TYR | B | 1212 | 21.945 | 33.515 | 16.177 | 1.00 | 65.12 | B |
| 5351 | N | THR | B | 1213 | 21.733 | 35.248 | 14.683 | 1.00 | 65.66 | B |
| 5352 | CA | THR | B | 1213 | 20.365 | 35.552 | 14.997 | 1.00 | 67.41 | B |
| 5353 | CB | THR | B | 1213 | 20.097 | 37.043 | 15.338 | 1.00 | 67.35 | B |
| 5354 | OG1 | THR | B | 1213 | 20.906 | 37.922 | 14.506 | 1.00 | 67.08 | B |
| 5355 | CG2 | THR | B | 1213 | 20.405 | 37.333 | 16.912 | 1.00 | 69.45 | B |
| 5356 | C | THR | B | 1213 | 19.808 | 34.992 | 13.694 | 1.00 | 68.44 | B |
| 5357 | O | THR | B | 1213 | 20.479 | 35.106 | 12.643 | 1.00 | 70.13 | B |
| 5358 | N | LEU | B | 1214 | 18.736 | 34.197 | 13.801 | 1.00 | 68.80 | B |
| 5359 | CA | LEU | B | 1214 | 18.170 | 33.498 | 12.666 | 1.00 | 66.92 | B |
| 5360 | CB | LEU | B | 1214 | 17.785 | 32.075 | 13.083 | 1.00 | 65.18 | B |
| 5361 | CG | LEU | B | 1214 | 18.769 | 31.137 | 13.852 | 1.00 | 56.00 | B |
| 5362 | CD1 | LEU | B | 1214 | 18.138 | 29.763 | 14.036 | 1.00 | 44.91 | B |
| 5363 | CD2 | LEU | B | 1214 | 20.041 | 31.003 | 13.175 | 1.00 | 48.70 | B |
| 5364 | C | LEU | B | 1214 | 16.941 | 34.350 | 12.252 | 1.00 | 68.44 | B |
| 5365 | O | LEU | B | 1214 | 15.877 | 34.270 | 12.949 | 1.00 | 69.13 | B |
| 5366 | N | GLU | B | 1215 | 17.139 | 35.250 | 11.252 | 1.00 | 67.69 | B |
| 5367 | CA | GLU | B | 1215 | 16.035 | 35.816 | 10.425 | 1.00 | 67.68 | B |
| 5368 | CB | GLU | B | 1215 | 16.500 | 36.956 | 9.513 | 1.00 | 69.06 | B |
| 5369 | CG | GLU | B | 1215 | 15.380 | 37.514 | 8.489 | 1.00 | 65.47 | B |
| 5370 | CD | GLU | B | 1215 | 16.002 | 38.296 | 7.246 | 1.00 | 60.75 | B |
| 5371 | OE1 | GLU | B | 1215 | 15.206 | 38.764 | 6.439 | 1.00 | 48.55 | B |
| 5372 | OE2 | GLU | B | 1215 | 17.260 | 38.542 | 7.080 | 1.00 | 49.32 | B |
| 5373 | C | GLU | B | 1215 | 15.460 | 34.681 | 9.535 | 1.00 | 70.70 | B |
| 5374 | O | GLU | B | 1215 | 15.775 | 34.608 | 8.283 | 1.00 | 71.16 | B |
| 5375 | N | PHE | B | 1216 | 14.684 | 33.783 | 10.181 | 1.00 | 69.96 | B |
| 5376 | CA | PHE | B | 1216 | 14.045 | 32.720 | 9.497 | 1.00 | 68.74 | B |
| 5377 | CB | PHE | B | 1216 | 13.150 | 31.935 | 10.476 | 1.00 | 68.69 | B |
| 5378 | CG | PHE | B | 1216 | 13.890 | 30.858 | 11.279 | 1.00 | 71.28 | B |
| 5379 | CD1 | PHE | B | 1216 | 14.429 | 29.743 | 10.683 | 1.00 | 71.44 | B |
| 5380 | CD2 | PHE | B | 1216 | 14.045 | 30.955 | 12.664 | 1.00 | 75.22 | B |
| 5381 | CE1 | PHE | B | 1216 | 15.150 | 28.780 | 11.486 | 1.00 | 74.37 | B |
| 5382 | CE2 | PHE | B | 1216 | 14.763 | 29.948 | 13.458 | 1.00 | 70.89 | B |
| 5383 | CZ | PHE | B | 1216 | 15.280 | 28.919 | 12.907 | 1.00 | 66.63 | B |
| 5384 | C | PHE | B | 1216 | 13.243 | 33.509 | 8.426 | 1.00 | 70.15 | B |
| 5385 | O | PHE | B | 1216 | 13.045 | 34.730 | 8.549 | 1.00 | 69.62 | B |
| 5386 | N | ARG | B | 1217 | 12.767 | 32.835 | 7.377 | 1.00 | 70.27 | B |
| 5387 | CA | ARG | B | 1217 | 12.174 | 33.503 | 6.321 | 1.00 | 68.74 | B |
| 5388 | CB | ARG | B | 1217 | 13.311 | 33.931 | 5.376 | 1.00 | 68.58 | B |
| 5389 | CG | ARG | B | 1217 | 13.515 | 35.399 | 5.424 | 1.00 | 67.67 | B |
| 5390 | CD | ARG | B | 1217 | 12.283 | 36.121 | 4.810 | 1.00 | 71.88 | B |
| 5391 | NE | ARG | B | 1217 | 11.127 | 36.351 | 5.708 | 1.00 | 76.95 | B |
| 5392 | CZ | ARG | B | 1217 | 10.074 | 37.175 | 5.513 | 1.00 | 75.51 | B |
| 5393 | NH1 | ARG | B | 1217 | 9.937 | 37.925 | 4.414 | 1.00 | 74.37 | B |
| 5394 | NH2 | ARG | B | 1217 | 9.139 | 37.256 | 6.453 | 1.00 | 69.46 | B |
| 5395 | C | ARG | B | 1217 | 11.218 | 32.546 | 5.679 | 1.00 | 69.39 | B |
| 5396 | O | ARG | B | 1217 | 11.657 | 31.464 | 5.399 | 1.00 | 68.93 | B |
| 5397 | N | SER | B | 1218 | 9.963 | 32.955 | 5.366 | 1.00 | 69.41 | B |
| 5398 | CA | SER | B | 1218 | 8.954 | 31.992 | 4.956 | 1.00 | 69.34 | B |
| 5399 | CB | SER | B | 1218 | 7.869 | 32.461 | 3.977 | 1.00 | 70.69 | B |
| 5400 | OG | SER | B | 1218 | 7.155 | 31.266 | 3.526 | 1.00 | 64.46 | B |
| 5401 | C | SER | B | 1218 | 9.556 | 30.879 | 4.225 | 1.00 | 69.87 | B |
| 5402 | O | SER | B | 1218 | 9.981 | 31.052 | 3.069 | 1.00 | 69.54 | B |
| 5403 | N | GLY | B | 1219 | 9.527 | 29.734 | 4.917 | 1.00 | 69.51 | B |
| 5404 | CA | GLY | B | 1219 | 10.043 | 28.516 | 4.383 | 1.00 | 66.93 | B |
| 5405 | C | GLY | B | 1219 | 11.538 | 28.520 | 4.707 | 1.00 | 64.85 | B |
| 5406 | O | GLY | B | 1219 | 12.110 | 27.456 | 4.928 | 1.00 | 67.53 | B |
| 5407 | N | LYS | B | 1220 | 12.226 | 29.652 | 4.727 | 1.00 | 61.90 | B |
| 5408 | CA | LYS | B | 1220 | 13.706 | 29.565 | 5.060 | 1.00 | 60.24 | B |
| 5409 | CB | LYS | B | 1220 | 14.605 | 29.835 | 3.823 | 1.00 | 56.47 | B |
| 5410 | CG | LYS | B | 1220 | 13.765 | 29.325 | 2.459 | 1.00 | 54.40 | B |
| 5411 | CD | LYS | B | 1220 | 14.812 | 28.591 | 1.458 | 1.00 | 55.73 | B |
| 5412 | CE | LYS | B | 1220 | 14.662 | 28.817 | −0.092 | 1.00 | 44.67 | B |
| 5413 | NZ | LYS | B | 1220 | 16.103 | 28.789 | −0.758 | 1.00 | 41.31 | B |
| 5414 | C | LYS | B | 1220 | 14.244 | 30.076 | 6.489 | 1.00 | 61.77 | B |
| 5415 | O | LYS | B | 1220 | 13.517 | 30.424 | 7.441 | 1.00 | 59.14 | B |
| 5416 | N | VAL | B | 1221 | 15.535 | 29.870 | 6.662 | 1.00 | 62.06 | B |
| 5417 | CA | VAL | B | 1221 | 16.215 | 30.646 | 7.568 | 1.00 | 61.04 | B |
| 5418 | CB | VAL | B | 1221 | 16.791 | 29.810 | 8.619 | 1.00 | 61.71 | B |
| 5419 | CG1 | VAL | B | 1221 | 17.029 | 28.515 | 8.081 | 1.00 | 64.97 | B |
| 5420 | CG2 | VAL | B | 1221 | 18.105 | 30.439 | 9.225 | 1.00 | 63.56 | B |
| 5421 | C | VAL | B | 1221 | 17.251 | 31.253 | 6.718 | 1.00 | 59.72 | B |
| 5422 | O | VAL | B | 1221 | 17.954 | 30.561 | 5.990 | 1.00 | 55.38 | B |
| 5423 | N | ALA | B | 1222 | 17.172 | 32.588 | 6.730 | 1.00 | 62.54 | B |
| 5424 | CA | ALA | B | 1222 | 18.310 | 33.563 | 6.549 | 1.00 | 63.67 | B |
| 5425 | CB | ALA | B | 1222 | 17.939 | 34.850 | 5.694 | 1.00 | 61.50 | B |
| 5426 | C | ALA | B | 1222 | 18.819 | 33.955 | 7.976 | 1.00 | 64.35 | B |
| 5427 | O | ALA | B | 1222 | 18.155 | 34.694 | 8.799 | 1.00 | 63.55 | B |
| 5428 | N | PHE | B | 1223 | 20.045 | 33.444 | 8.173 | 1.00 | 63.98 | B |
| 5429 | CA | PHE | B | 1223 | 20.964 | 33.668 | 9.289 | 1.00 | 61.74 | B |
| 5430 | CB | PHE | B | 1223 | 22.242 | 32.817 | 9.060 | 1.00 | 60.54 | B |
| 5431 | CG | PHE | B | 1223 | 22.002 | 31.369 | 8.870 | 1.00 | 57.18 | B |
| 5432 | CD1 | PHE | B | 1223 | 21.004 | 30.664 | 9.637 | 1.00 | 56.35 | B |
| 5433 | CD2 | PHE | B | 1223 | 22.759 | 30.653 | 7.899 | 1.00 | 61.33 | B |
| 5434 | CE1 | PHE | B | 1223 | 20.791 | 29.239 | 9.471 | 1.00 | 57.87 | B |
| 5435 | CE2 | PHE | B | 1223 | 22.560 | 29.129 | 7.713 | 1.00 | 59.65 | B |
| 5436 | CZ | PHE | B | 1223 | 21.580 | 28.460 | 8.493 | 1.00 | 54.24 | B |
| 5437 | C | PHE | B | 1223 | 21.495 | 35.003 | 9.411 | 1.00 | 61.59 | B |
| 5438 | O | PHE | B | 1223 | 22.263 | 35.394 | 8.503 | 1.00 | 62.01 | B |
| 5439 | N | ARG | B | 1224 | 21.222 | 35.676 | 10.543 | 1.00 | 61.72 | B |
| 5440 | CA | ARG | B | 1224 | 22.027 | 36.850 | 10.888 | 1.00 | 62.11 | B |
| 5441 | CB | ARG | B | 1224 | 21.572 | 37.458 | 12.103 | 1.00 | 60.10 | B |
| 5442 | CG | ARG | B | 1224 | 20.979 | 38.768 | 11.866 | 1.00 | 66.66 | B |
| 5443 | CD | ARG | B | 1224 | 21.987 | 39.902 | 11.497 | 1.00 | 75.37 | B |
| 5444 | NE | ARG | B | 1224 | 21.385 | 41.109 | 10.832 | 1.00 | 74.00 | B |
| 5445 | CZ | ARG | B | 1224 | 21.262 | 42.315 | 11.393 | 1.00 | 72.34 | B |
| 5446 | NH1 | ARG | B | 1224 | 21.648 | 42.608 | 12.644 | 1.00 | 68.75 | B |
| 5447 | NH2 | ARG | B | 1224 | 20.776 | 43.263 | 10.662 | 1.00 | 77.74 | B |
| 5448 | C | ARG | B | 1224 | 23.463 | 36.496 | 11.138 | 1.00 | 64.13 | B |
| 5449 | O | ARG | B | 1224 | 23.830 | 35.341 | 11.321 | 1.00 | 63.37 | B |
| 5450 | N | ASP | B | 1225 | 24.311 | 37.525 | 11.125 | 1.00 | 67.32 | B |
| 5451 | CA | ASP | B | 1225 | 25.705 | 37.417 | 11.582 | 1.00 | 68.20 | B |
| 5452 | CB | ASP | B | 1225 | 26.749 | 37.228 | 10.464 | 1.00 | 68.95 | B |
| 5453 | CG | ASP | B | 1225 | 27.579 | 38.547 | 10.064 | 1.00 | 73.18 | B |
| 5454 | OD1 | ASP | B | 1225 | 28.053 | 39.287 | 10.984 | 1.00 | 75.85 | B |
| 5455 | OD2 | ASP | B | 1225 | 27.849 | 38.757 | 8.812 | 1.00 | 70.10 | B |
| 5456 | C | ASP | B | 1225 | 25.912 | 38.615 | 12.351 | 1.00 | 69.73 | B |
| 5457 | O | ASP | B | 1225 | 25.271 | 39.663 | 12.120 | 1.00 | 71.24 | B |
| 5458 | N | CYS | B | 1226 | 26.843 | 38.442 | 13.251 | 1.00 | 72.12 | B |
| 5459 | CA | CYS | B | 1226 | 27.162 | 39.354 | 14.345 | 1.00 | 75.96 | B |
| 5460 | CB | CYS | B | 1226 | 28.003 | 38.574 | 15.483 | 1.00 | 76.99 | B |
| 5461 | SG | CYS | B | 1226 | 29.531 | 37.606 | 14.929 | 1.00 | 77.47 | B |
| 5462 | C | CYS | B | 1226 | 27.833 | 40.680 | 13.943 | 1.00 | 74.83 | B |
| 5463 | O | CYS | B | 1226 | 28.492 | 41.346 | 14.776 | 1.00 | 75.32 | B |
| 5464 | N | GLU | B | 1227 | 27.705 | 40.988 | 12.667 | 1.00 | 75.01 | B |
| 5465 | CA | GLU | B | 1227 | 28.210 | 42.205 | 12.070 | 1.00 | 75.65 | B |
| 5466 | CB | GLU | B | 1227 | 29.178 | 41.846 | 10.939 | 1.00 | 73.43 | B |
| 5467 | CG | GLU | B | 1227 | 30.603 | 41.316 | 11.346 | 1.00 | 73.46 | B |
| 5468 | CD | GLU | B | 1227 | 31.444 | 42.335 | 12.217 | 1.00 | 75.42 | B |
| 5469 | OE1 | GLU | B | 1227 | 32.717 | 42.404 | 12.012 | 1.00 | 73.44 | B |
| 5470 | OE2 | GLU | B | 1227 | 30.836 | 43.048 | 13.099 | 1.00 | 68.23 | B |
| 5471 | C | GLU | B | 1227 | 27.003 | 43.139 | 11.588 | 1.00 | 77.70 | B |
| 5472 | O | GLU | B | 1227 | 27.195 | 44.237 | 11.005 | 1.00 | 80.30 | B |
| 5473 | N | GLY | B | 1228 | 25.754 | 42.752 | 11.838 | 1.00 | 77.00 | B |
| 5474 | CA | GLY | B | 1228 | 24.699 | 43.254 | 10.951 | 1.00 | 75.83 | B |
| 5475 | C | GLY | B | 1228 | 24.705 | 42.548 | 9.551 | 1.00 | 75.08 | B |
| 5476 | O | GLY | B | 1228 | 23.647 | 42.444 | 8.837 | 1.00 | 72.10 | B |
| 5477 | N | ARG | B | 1229 | 25.900 | 42.071 | 9.157 | 1.00 | 74.97 | B |
| 5478 | CA | ARG | B | 1229 | 26.034 | 41.274 | 7.895 | 1.00 | 74.64 | B |
| 5479 | CB | ARG | B | 1229 | 27.538 | 41.134 | 7.475 | 1.00 | 75.05 | B |
| 5480 | CG | ARG | B | 1229 | 27.963 | 41.953 | 6.241 | 1.00 | 73.86 | B |
| 5481 | CD | ARG | B | 1229 | 29.091 | 43.033 | 6.383 | 1.00 | 68.40 | B |
| 5482 | NE | ARG | B | 1229 | 30.151 | 42.892 | 7.417 | 1.00 | 74.54 | B |
| 5483 | CZ | ARG | B | 1229 | 31.446 | 42.464 | 7.320 | 1.00 | 76.47 | B |
| 5484 | NH1 | ARG | B | 1229 | 31.954 | 42.011 | 6.142 | 1.00 | 78.34 | B |
| 5485 | NH2 | ARG | B | 1229 | 32.285 | 42.469 | 8.431 | 1.00 | 65.02 | B |
| 5486 | C | ARG | B | 1229 | 25.201 | 39.919 | 8.018 | 1.00 | 72.97 | B |
| 5487 | O | ARG | B | 1229 | 24.358 | 39.762 | 8.971 | 1.00 | 76.06 | B |
| 5488 | N | TYR | B | 1230 | 25.369 | 38.998 | 7.093 | 1.00 | 68.22 | B |
| 5489 | CA | TYR | B | 1230 | 24.412 | 37.922 | 6.923 | 1.00 | 66.47 | B |
| 5490 | CB | TYR | B | 1230 | 23.118 | 38.304 | 6.120 | 1.00 | 65.13 | B |
| 5491 | CG | TYR | B | 1230 | 21.950 | 39.143 | 6.583 | 1.00 | 58.96 | B |
| 5492 | CD1 | TYR | B | 1230 | 21.998 | 40.480 | 6.603 | 1.00 | 53.96 | B |
| 5493 | CE1 | TYR | B | 1230 | 20.693 | 41.329 | 6.963 | 1.00 | 63.45 | B |
| 5494 | CD2 | TYR | B | 1230 | 20.680 | 38.543 | 6.787 | 1.00 | 63.41 | B |
| 5495 | CE2 | TYR | B | 1230 | 19.386 | 39.301 | 7.071 | 1.00 | 58.78 | B |
| 5496 | CZ | TYR | B | 1230 | 19.409 | 40.668 | 7.150 | 1.00 | 64.14 | B |
| 5497 | OH | TYR | B | 1230 | 18.196 | 41.340 | 7.463 | 1.00 | 63.39 | B |
| 5498 | C | TYR | B | 1230 | 25.189 | 36.999 | 5.981 | 1.00 | 66.58 | B |
| 5499 | O | TYR | B | 1230 | 25.765 | 37.483 | 5.008 | 1.00 | 63.48 | B |
| 5500 | N | LEU | B | 1231 | 25.144 | 35.691 | 6.287 | 1.00 | 67.86 | B |
| 5501 | CA | LEU | B | 1231 | 26.003 | 34.603 | 5.740 | 1.00 | 69.53 | B |
| 5502 | CB | LEU | B | 1231 | 26.298 | 33.560 | 6.897 | 1.00 | 68.55 | B |
| 5503 | CG | LEU | B | 1231 | 27.496 | 33.812 | 7.912 | 1.00 | 68.20 | B |
| 5504 | CD1 | LEU | B | 1231 | 27.121 | 33.771 | 9.492 | 1.00 | 60.77 | B |
| 5505 | CD2 | LEU | B | 1231 | 28.908 | 32.961 | 7.653 | 1.00 | 61.08 | B |
| 5506 | C | LEU | B | 1231 | 25.547 | 33.904 | 4.356 | 1.00 | 69.56 | B |
| 5507 | O | LEU | B | 1231 | 24.344 | 33.737 | 4.128 | 1.00 | 71.79 | B |
| 5508 | N | ALA | B | 1232 | 26.452 | 33.438 | 3.476 | 1.00 | 67.19 | B |
| 5509 | CA | ALA | B | 1232 | 25.971 | 33.071 | 2.160 | 1.00 | 66.38 | B |
| 5510 | CB | ALA | B | 1232 | 25.319 | 34.348 | 1.460 | 1.00 | 66.64 | B |
| 5511 | C | ALA | B | 1232 | 27.026 | 32.424 | 1.255 | 1.00 | 66.57 | B |
| 5512 | O | ALA | B | 1232 | 28.233 | 32.868 | 1.237 | 1.00 | 65.57 | B |
| 5513 | N | PRO | B | 1233 | 26.593 | 31.378 | 0.456 | 1.00 | 66.30 | B |
| 5514 | CD | PRO | B | 1233 | 25.254 | 30.760 | 0.445 | 1.00 | 64.69 | B |
| 5515 | CA | PRO | B | 1233 | 27.424 | 30.749 | −0.619 | 1.00 | 65.82 | B |
| 5516 | CB | PRO | B | 1233 | 26.354 | 30.429 | −1.635 | 1.00 | 65.13 | B |
| 5517 | CG | PRO | B | 1233 | 25.265 | 29.904 | −0.787 | 1.00 | 62.08 | B |
| 5518 | C | PRO | B | 1233 | 28.493 | 31.704 | −1.261 | 1.00 | 65.95 | B |
| 5519 | O | PRO | B | 1233 | 28.137 | 32.809 | −1.470 | 1.00 | 67.40 | B |
| 5520 | N | SER | B | 1234 | 29.762 | 31.293 | −1.452 | 1.00 | 65.61 | B |
| 5521 | CA | SER | B | 1234 | 30.860 | 31.971 | −2.167 | 1.00 | 63.45 | B |
| 5522 | CB | SER | B | 1234 | 32.077 | 32.063 | −1.246 | 1.00 | 63.22 | B |
| 5523 | OG | SER | B | 1234 | 33.301 | 32.364 | −1.952 | 1.00 | 62.69 | B |
| 5524 | C | SER | B | 1234 | 31.314 | 31.080 | −3.399 | 1.00 | 63.92 | B |
| 5525 | O | SER | B | 1234 | 30.460 | 30.609 | −4.202 | 1.00 | 62.01 | B |
| 5526 | N | GLY | B | 1235 | 32.655 | 30.885 | −3.530 | 1.00 | 61.90 | B |
| 5527 | CA | GLY | B | 1235 | 33.252 | 29.749 | −4.265 | 1.00 | 59.42 | B |
| 5528 | C | GLY | B | 1235 | 32.316 | 28.878 | −5.062 | 1.00 | 56.69 | B |
| 5529 | O | GLY | B | 1235 | 31.159 | 29.181 | −5.143 | 1.00 | 52.94 | B |
| 5530 | N | PRO | B | 1236 | 32.873 | 27.905 | −5.808 | 1.00 | 58.47 | B |
| 5531 | CD | PRO | B | 1236 | 34.144 | 28.108 | −6.543 | 1.00 | 60.27 | B |
| 5532 | CA | PRO | B | 1236 | 32.281 | 26.591 | −6.170 | 1.00 | 59.57 | B |
| 5533 | CB | PRO | B | 1236 | 33.271 | 26.025 | −7.235 | 1.00 | 61.04 | B |
| 5534 | CG | PRO | B | 1236 | 33.976 | 27.230 | −7.809 | 1.00 | 59.35 | B |
| 5535 | C | PRO | B | 1236 | 32.290 | 25.658 | −4.953 | 1.00 | 60.59 | B |
| 5536 | O | PRO | B | 1236 | 31.689 | 24.520 | −5.020 | 1.00 | 57.74 | B |
| 5537 | N | SER | B | 1237 | 33.018 | 26.122 | −3.880 | 1.00 | 61.47 | B |
| 5538 | CA | SER | B | 1237 | 33.140 | 25.320 | −2.665 | 1.00 | 62.54 | B |
| 5539 | CB | SER | B | 1237 | 34.569 | 25.212 | −2.004 | 1.00 | 61.62 | B |
| 5540 | OG | SER | B | 1237 | 35.288 | 26.456 | −1.878 | 1.00 | 63.60 | B |
| 5541 | C | SER | B | 1237 | 32.055 | 25.923 | −1.855 | 1.00 | 62.28 | B |
| 5542 | O | SER | B | 1237 | 31.995 | 25.790 | −0.642 | 1.00 | 67.38 | B |
| 5543 | N | GLY | B | 1238 | 31.091 | 26.495 | −2.542 | 1.00 | 61.04 | B |
| 5544 | CA | GLY | B | 1238 | 30.084 | 27.354 | −1.860 | 1.00 | 61.43 | B |
| 5545 | C | GLY | B | 1238 | 30.634 | 27.890 | −0.511 | 1.00 | 60.43 | B |
| 5546 | O | GLY | B | 1238 | 29.957 | 27.886 | 0.476 | 1.00 | 58.97 | B |
| 5547 | N | THR | B | 1239 | 31.891 | 28.295 | −0.461 | 1.00 | 59.86 | B |
| 5548 | CA | THR | B | 1239 | 32.466 | 28.610 | 0.888 | 1.00 | 61.17 | B |
| 5549 | CB | THR | B | 1239 | 33.977 | 29.181 | 0.763 | 1.00 | 63.48 | B |
| 5550 | OG1 | THR | B | 1239 | 34.681 | 28.731 | −0.505 | 1.00 | 60.75 | B |
| 5551 | CG2 | THR | B | 1239 | 34.810 | 29.051 | 2.253 | 1.00 | 60.50 | B |
| 5552 | C | THR | B | 1239 | 31.561 | 29.595 | 1.770 | 1.00 | 59.30 | B |
| 5553 | O | THR | B | 1239 | 31.582 | 30.771 | 1.675 | 1.00 | 59.26 | B |
| 5554 | N | LEU | B | 1240 | 30.696 | 29.114 | 2.571 | 1.00 | 58.94 | B |
| 5555 | CA | LEU | B | 1240 | 29.931 | 30.026 | 3.331 | 1.00 | 61.17 | B |
| 5556 | CB | LEU | B | 1240 | 28.781 | 29.231 | 3.954 | 1.00 | 60.08 | B |
| 5557 | CG | LEU | B | 1240 | 28.791 | 27.843 | 3.348 | 1.00 | 56.94 | B |
| 5558 | CD1 | LEU | B | 1240 | 28.241 | 26.789 | 4.342 | 1.00 | 50.19 | B |
| 5559 | CD2 | LEU | B | 1240 | 28.018 | 27.923 | 1.984 | 1.00 | 64.18 | B |
| 5560 | C | LEU | B | 1240 | 30.774 | 30.735 | 4.454 | 1.00 | 62.94 | B |
| 5561 | O | LEU | B | 1240 | 31.492 | 30.018 | 5.273 | 1.00 | 66.34 | B |
| 5562 | N | LYS | B | 1244 | 28.212 | 40.485 | 3.577 | 1.00 | 71.54 | B |
| 5563 | CA | LYS | B | 1244 | 28.266 | 41.794 | 3.000 | 1.00 | 72.69 | B |
| 5564 | CB | LYS | B | 1244 | 29.449 | 42.010 | 2.040 | 1.00 | 73.46 | B |
| 5565 | CG | LYS | B | 1244 | 29.906 | 40.799 | 1.068 | 1.00 | 73.56 | B |
| 5566 | CD | LYS | B | 1244 | 31.267 | 41.254 | 0.301 | 1.00 | 74.39 | B |
| 5567 | CE | LYS | B | 1244 | 32.662 | 41.018 | 1.191 | 1.00 | 71.86 | B |
| 5568 | NZ | LYS | B | 1244 | 33.638 | 42.140 | 0.863 | 1.00 | 68.59 | B |
| 5569 | C | LYS | B | 1244 | 26.931 | 41.906 | 2.332 | 1.00 | 73.62 | B |
| 5570 | O | LYS | B | 1244 | 26.795 | 41.800 | 1.052 | 1.00 | 76.06 | B |
| 5571 | N | ALA | B | 1245 | 25.908 | 42.021 | 3.195 | 1.00 | 71.23 | B |
| 5572 | CA | ALA | B | 1245 | 24.574 | 42.056 | 2.683 | 1.00 | 68.40 | B |
| 5573 | CB | ALA | B | 1245 | 23.916 | 40.619 | 2.725 | 1.00 | 68.20 | B |
| 5574 | C | ALA | B | 1245 | 23.773 | 43.053 | 3.487 | 1.00 | 67.74 | B |
| 5575 | O | ALA | B | 1245 | 23.660 | 42.952 | 4.770 | 1.00 | 67.65 | B |
| 5576 | N | THR | B | 1246 | 23.202 | 44.012 | 2.755 | 1.00 | 65.07 | B |
| 5577 | CA | THR | B | 1246 | 22.270 | 44.929 | 3.387 | 1.00 | 62.60 | B |
| 5578 | CB | THR | B | 1246 | 22.001 | 46.266 | 2.532 | 1.00 | 61.77 | B |
| 5579 | OG1 | THR | B | 1246 | 23.188 | 46.604 | 1.788 | 1.00 | 55.70 | B |
| 5580 | CG2 | THR | B | 1246 | 21.534 | 47.455 | 3.428 | 1.00 | 60.20 | B |
| 5581 | C | THR | B | 1246 | 21.028 | 44.079 | 3.644 | 1.00 | 62.40 | B |
| 5582 | O | THR | B | 1246 | 20.920 | 43.487 | 4.751 | 1.00 | 63.06 | B |
| 5583 | N | LYS | B | 1247 | 20.124 | 44.036 | 2.642 | 1.00 | 61.22 | B |
| 5584 | CA | LYS | B | 1247 | 18.801 | 43.446 | 2.816 | 1.00 | 60.96 | B |
| 5585 | CB | LYS | B | 1247 | 17.713 | 44.099 | 1.974 | 1.00 | 60.47 | B |
| 5586 | CG | LYS | B | 1247 | 16.298 | 43.438 | 2.261 | 1.00 | 54.10 | B |
| 5587 | CD | LYS | B | 1247 | 15.279 | 44.487 | 2.409 | 1.00 | 54.54 | B |
| 5588 | CE | LYS | B | 1247 | 15.701 | 45.979 | 2.022 | 1.00 | 48.70 | B |
| 5589 | NZ | LYS | B | 1247 | 14.519 | 46.912 | 1.754 | 1.00 | 43.98 | B |
| 5590 | C | LYS | B | 1247 | 18.962 | 42.007 | 2.461 | 1.00 | 61.67 | B |
| 5591 | O | LYS | B | 1247 | 19.885 | 41.726 | 1.709 | 1.00 | 60.26 | B |
| 5592 | N | VAL | B | 1248 | 18.163 | 41.107 | 3.100 | 1.00 | 63.43 | B |
| 5593 | CA | VAL | B | 1248 | 18.139 | 39.638 | 2.698 | 1.00 | 63.99 | B |
| 5594 | CB | VAL | B | 1248 | 17.021 | 38.806 | 3.547 | 1.00 | 62.13 | B |
| 5595 | CG1 | VAL | B | 1248 | 15.560 | 38.940 | 3.015 | 1.00 | 65.30 | B |
| 5596 | CG2 | VAL | B | 1248 | 17.291 | 37.444 | 3.574 | 1.00 | 54.18 | B |
| 5597 | C | VAL | B | 1248 | 17.988 | 39.601 | 1.093 | 1.00 | 65.09 | B |
| 5598 | O | VAL | B | 1248 | 17.047 | 40.234 | 0.502 | 1.00 | 65.84 | B |
| 5599 | N | GLY | B | 1249 | 18.918 | 38.927 | 0.406 | 1.00 | 66.31 | B |
| 5600 | CA | GLY | B | 1249 | 18.983 | 38.836 | −1.121 | 1.00 | 65.33 | B |
| 5601 | C | GLY | B | 1249 | 18.460 | 37.423 | −1.473 | 1.00 | 67.14 | B |
| 5602 | O | GLY | B | 1249 | 17.886 | 36.695 | −0.614 | 1.00 | 67.68 | B |
| 5603 | N | LYS | B | 1250 | 18.641 | 37.013 | −2.713 | 1.00 | 66.62 | B |
| 5604 | CA | LYS | B | 1250 | 18.063 | 35.818 | −3.208 | 1.00 | 64.94 | B |
| 5605 | CB | LYS | B | 1250 | 18.130 | 35.906 | −4.718 | 1.00 | 66.64 | B |
| 5606 | CG | LYS | B | 1250 | 19.302 | 36.895 | −5.404 | 1.00 | 64.78 | B |
| 5607 | CD | LYS | B | 1250 | 19.153 | 36.798 | −7.008 | 1.00 | 58.62 | B |
| 5608 | CE | LYS | B | 1250 | 18.009 | 37.636 | −7.595 | 1.00 | 50.08 | B |
| 5609 | NZ | LYS | B | 1250 | 16.681 | 36.811 | −7.687 | 1.00 | 49.90 | B |
| 5610 | C | LYS | B | 1250 | 18.835 | 34.506 | −2.754 | 1.00 | 67.65 | B |
| 5611 | O | LYS | B | 1250 | 18.258 | 33.381 | −2.851 | 1.00 | 65.76 | B |
| 5612 | N | ASP | B | 1251 | 20.125 | 34.661 | −2.349 | 1.00 | 67.20 | B |
| 5613 | CA | ASP | B | 1251 | 20.983 | 33.578 | −1.766 | 1.00 | 67.10 | B |
| 5614 | CB | ASP | B | 1251 | 22.468 | 33.891 | −1.998 | 1.00 | 69.09 | B |
| 5615 | CG | ASP | B | 1251 | 22.699 | 35.420 | −2.355 | 1.00 | 74.14 | B |
| 5616 | OD1 | ASP | B | 1251 | 22.685 | 35.850 | −3.569 | 1.00 | 75.10 | B |
| 5617 | OD2 | ASP | B | 1251 | 22.860 | 36.207 | −1.378 | 1.00 | 80.61 | B |
| 5618 | C | ASP | B | 1251 | 20.773 | 33.240 | −0.273 | 1.00 | 65.83 | B |
| 5619 | O | ASP | B | 1251 | 20.271 | 32.145 | 0.053 | 1.00 | 62.43 | B |
| 5620 | N | GLU | B | 1252 | 21.208 | 34.177 | 0.584 | 1.00 | 65.16 | B |
| 5621 | CA | GLU | B | 1252 | 21.265 | 34.111 | 2.100 | 1.00 | 65.85 | B |
| 5622 | CB | GLU | B | 1252 | 21.003 | 35.513 | 2.687 | 1.00 | 63.70 | B |
| 5623 | CG | GLU | B | 1252 | 22.062 | 36.623 | 2.461 | 1.00 | 71.12 | B |
| 5624 | CD | GLU | B | 1252 | 21.893 | 37.336 | 1.079 | 1.00 | 74.74 | B |
| 5625 | OE1 | GLU | B | 1252 | 22.394 | 38.469 | 0.939 | 1.00 | 71.11 | B |
| 5626 | OE2 | GLU | B | 1252 | 21.219 | 36.763 | 0.144 | 1.00 | 78.95 | B |
| 5627 | C | GLU | B | 1252 | 20.371 | 33.155 | 2.980 | 1.00 | 65.26 | B |
| 5628 | O | GLU | B | 1252 | 20.498 | 33.196 | 4.223 | 1.00 | 65.50 | B |
| 5629 | N | LEU | B | 1253 | 19.483 | 32.381 | 2.329 | 1.00 | 63.81 | B |
| 5630 | CA | LEU | B | 1253 | 18.240 | 31.829 | 2.855 | 1.00 | 63.61 | B |
| 5631 | CB | LEU | B | 1253 | 17.064 | 32.060 | 1.853 | 1.00 | 62.78 | B |
| 5632 | CG | LEU | B | 1253 | 15.941 | 33.065 | 1.522 | 1.00 | 62.78 | B |
| 5633 | CD1 | LEU | B | 1253 | 14.670 | 33.031 | 2.498 | 1.00 | 68.28 | B |
| 5634 | CD2 | LEU | B | 1253 | 16.380 | 34.575 | 1.253 | 1.00 | 63.71 | B |
| 5635 | C | LEU | B | 1253 | 18.477 | 30.335 | 2.853 | 1.00 | 63.79 | B |
| 5636 | O | LEU | B | 1253 | 18.784 | 29.783 | 1.804 | 1.00 | 65.56 | B |
| 5637 | N | PHE | B | 1254 | 18.311 | 29.638 | 3.964 | 1.00 | 64.25 | B |
| 5638 | CA | PHE | B | 1254 | 18.765 | 28.231 | 3.967 | 1.00 | 65.04 | B |
| 5639 | CB | PHE | B | 1254 | 19.896 | 28.068 | 4.959 | 1.00 | 63.16 | B |
| 5640 | CG | PHE | B | 1254 | 21.316 | 28.453 | 4.381 | 1.00 | 69.08 | B |
| 5641 | CD1 | PHE | B | 1254 | 21.756 | 27.966 | 3.081 | 1.00 | 64.84 | B |
| 5642 | CD2 | PHE | B | 1254 | 22.220 | 29.354 | 5.148 | 1.00 | 64.20 | B |
| 5643 | CE1 | PHE | B | 1254 | 23.071 | 28.342 | 2.574 | 1.00 | 63.94 | B |
| 5644 | CE2 | PHE | B | 1254 | 23.558 | 29.693 | 4.663 | 1.00 | 57.66 | B |
| 5645 | CZ | PHE | B | 1254 | 24.006 | 29.189 | 3.406 | 1.00 | 57.11 | B |
| 5646 | C | PHE | B | 1254 | 17.651 | 27.179 | 4.198 | 1.00 | 66.15 | B |
| 5647 | O | PHE | B | 1254 | 16.690 | 27.430 | 5.001 | 1.00 | 68.23 | B |
| 5648 | N | ALA | B | 1255 | 17.721 | 26.042 | 3.472 | 1.00 | 64.52 | B |
| 5649 | CA | ALA | B | 1255 | 16.832 | 24.915 | 3.734 | 1.00 | 60.37 | B |
| 5650 | CB | ALA | B | 1255 | 16.942 | 24.015 | 2.605 | 1.00 | 62.62 | B |
| 5651 | C | ALA | B | 1255 | 17.439 | 24.233 | 4.887 | 1.00 | 59.50 | B |
| 5652 | O | ALA | B | 1255 | 18.594 | 23.825 | 4.784 | 1.00 | 60.72 | B |
| 5653 | N | LEU | B | 1256 | 16.688 | 24.063 | 5.961 | 1.00 | 58.16 | B |
| 5654 | CA | LEU | B | 1256 | 17.099 | 23.335 | 7.173 | 1.00 | 55.49 | B |
| 5655 | CB | LEU | B | 1256 | 16.719 | 24.123 | 8.465 | 1.00 | 53.65 | B |
| 5656 | CG | LEU | B | 1256 | 17.673 | 25.374 | 8.586 | 1.00 | 52.33 | B |
| 5657 | CD1 | LEU | B | 1256 | 17.382 | 26.394 | 9.879 | 1.00 | 43.49 | B |
| 5658 | CD2 | LEU | B | 1256 | 19.156 | 25.059 | 8.315 | 1.00 | 40.69 | B |
| 5659 | C | LEU | B | 1256 | 16.374 | 22.037 | 7.180 | 1.00 | 54.68 | B |
| 5660 | O | LEU | B | 1256 | 15.230 | 22.024 | 7.634 | 1.00 | 54.86 | B |
| 5661 | N | GLU | B | 1257 | 16.990 | 20.944 | 6.698 | 1.00 | 55.28 | B |
| 5662 | CA | GLU | B | 1257 | 16.331 | 19.572 | 6.836 | 1.00 | 55.55 | B |
| 5663 | CB | GLU | B | 1257 | 16.379 | 18.812 | 5.561 | 1.00 | 56.54 | B |
| 5664 | CG | GLU | B | 1257 | 17.517 | 19.171 | 4.759 | 1.00 | 58.88 | B |
| 5665 | CD | GLU | B | 1257 | 17.140 | 19.206 | 3.343 | 1.00 | 64.12 | B |
| 5666 | OE1 | GLU | B | 1257 | 17.932 | 18.460 | 2.694 | 1.00 | 66.73 | B |
| 5667 | OE2 | GLU | B | 1257 | 16.107 | 19.927 | 2.938 | 1.00 | 55.38 | B |
| 5668 | C | GLU | B | 1257 | 16.829 | 18.598 | 7.936 | 1.00 | 55.93 | B |
| 5669 | O | GLU | B | 1257 | 18.040 | 18.523 | 8.141 | 1.00 | 57.66 | B |
| 5670 | N | GLN | B | 1258 | 15.906 | 17.913 | 8.635 | 1.00 | 53.64 | B |
| 5671 | CA | GLN | B | 1258 | 16.198 | 16.780 | 9.430 | 1.00 | 53.86 | B |
| 5672 | CB | GLN | B | 1258 | 14.921 | 15.951 | 9.661 | 1.00 | 53.27 | B |
| 5673 | CG | GLN | B | 1258 | 13.725 | 16.759 | 10.247 | 1.00 | 54.42 | B |
| 5674 | CD | GLN | B | 1258 | 12.444 | 16.035 | 10.588 | 1.00 | 52.44 | B |
| 5675 | OE1 | GLN | B | 1258 | 11.453 | 16.708 | 10.883 | 1.00 | 54.92 | B |
| 5676 | NE2 | GLN | B | 1258 | 12.412 | 14.711 | 10.477 | 1.00 | 48.80 | B |
| 5677 | C | GLN | B | 1258 | 17.282 | 15.937 | 8.751 | 1.00 | 55.40 | B |
| 5678 | O | GLN | B | 1258 | 17.224 | 15.579 | 7.551 | 1.00 | 56.48 | B |
| 5679 | N | SER | B | 1259 | 18.316 | 15.633 | 9.533 | 1.00 | 56.27 | B |
| 5680 | CA | SER | B | 1259 | 19.370 | 14.653 | 9.156 | 1.00 | 53.65 | B |
| 5681 | CB | SER | B | 1259 | 20.595 | 15.057 | 9.896 | 1.00 | 55.96 | B |
| 5682 | OG | SER | B | 1259 | 21.247 | 13.921 | 10.140 | 1.00 | 60.48 | B |
| 5683 | C | SER | B | 1259 | 18.959 | 13.350 | 9.691 | 1.00 | 50.46 | B |
| 5684 | O | SER | B | 1259 | 18.457 | 13.272 | 10.809 | 1.00 | 48.82 | B |
| 5685 | N | CYS | B | 1260 | 19.137 | 12.302 | 8.912 | 1.00 | 50.46 | B |
| 5686 | CA | CYS | B | 1260 | 18.695 | 10.952 | 9.416 | 1.00 | 51.51 | B |
| 5687 | CB | CYS | B | 1260 | 17.747 | 10.151 | 8.490 | 1.00 | 53.28 | B |
| 5688 | SG | CYS | B | 1260 | 16.107 | 10.795 | 8.181 | 1.00 | 50.55 | B |
| 5689 | C | CYS | B | 1260 | 19.897 | 10.125 | 9.805 | 1.00 | 48.69 | B |
| 5690 | O | CYS | B | 1260 | 20.947 | 10.357 | 9.198 | 1.00 | 47.86 | B |
| 5691 | N | ALA | B | 1261 | 19.762 | 9.350 | 10.865 | 1.00 | 43.86 | B |
| 5692 | CA | ALA | B | 1261 | 20.682 | 8.184 | 11.037 | 1.00 | 46.63 | B |
| 5693 | CB | ALA | B | 1261 | 20.166 | 7.075 | 12.163 | 1.00 | 45.05 | B |
| 5694 | C | ALA | B | 1261 | 21.154 | 7.394 | 9.782 | 1.00 | 48.10 | B |
| 5695 | O | ALA | B | 1261 | 20.352 | 7.003 | 8.843 | 1.00 | 47.89 | B |
| 5696 | N | GLN | B | 1262 | 22.481 | 7.160 | 9.811 | 1.00 | 47.86 | B |
| 5697 | CA | GLN | B | 1262 | 23.166 | 6.604 | 8.693 | 1.00 | 50.13 | B |
| 5698 | CB | GLN | B | 1262 | 23.987 | 7.695 | 7.842 | 1.00 | 48.76 | B |
| 5699 | CG | GLN | B | 1262 | 23.055 | 8.721 | 7.171 | 1.00 | 45.44 | B |
| 5700 | CD | GLN | B | 1262 | 23.644 | 9.723 | 6.078 | 1.00 | 48.23 | B |
| 5701 | OE1 | GLN | B | 1262 | 24.859 | 9.974 | 6.012 | 1.00 | 45.91 | B |
| 5702 | NE2 | GLN | B | 1262 | 22.733 | 10.279 | 5.209 | 1.00 | 38.52 | B |
| 5703 | C | GLN | B | 1262 | 23.976 | 5.522 | 9.375 | 1.00 | 50.73 | B |
| 5704 | O | GLN | B | 1262 | 24.649 | 5.750 | 10.395 | 1.00 | 51.73 | B |
| 5705 | N | VAL | B | 1263 | 23.796 | 4.311 | 8.852 | 1.00 | 51.73 | B |
| 5706 | CA | VAL | B | 1263 | 24.424 | 3.101 | 9.399 | 1.00 | 49.38 | B |
| 5707 | CB | VAL | B | 1263 | 23.439 | 2.282 | 10.236 | 1.00 | 47.37 | B |
| 5708 | CG1 | VAL | B | 1263 | 22.574 | 3.162 | 11.042 | 1.00 | 45.25 | B |
| 5709 | CG2 | VAL | B | 1263 | 22.491 | 1.495 | 9.423 | 1.00 | 50.88 | B |
| 5710 | C | VAL | B | 1263 | 25.003 | 2.449 | 8.165 | 1.00 | 50.25 | B |
| 5711 | O | VAL | B | 1263 | 24.552 | 2.806 | 7.037 | 1.00 | 47.08 | B |
| 5712 | N | VAL | B | 1264 | 26.197 | 1.907 | 8.421 | 1.00 | 52.80 | B |
| 5713 | CA | VAL | B | 1264 | 26.835 | 0.638 | 7.948 | 1.00 | 57.04 | B |
| 5714 | CB | VAL | B | 1264 | 28.213 | 0.541 | 8.739 | 1.00 | 55.28 | B |
| 5715 | CG1 | VAL | B | 1264 | 28.906 | −0.789 | 8.684 | 1.00 | 56.13 | B |
| 5716 | CG2 | VAL | B | 1264 | 29.156 | 1.649 | 8.480 | 1.00 | 52.61 | B |
| 5717 | C | VAL | B | 1264 | 26.017 | −0.597 | 8.499 | 1.00 | 59.69 | B |
| 5718 | O | VAL | B | 1264 | 25.599 | −0.541 | 9.679 | 1.00 | 64.42 | B |
| 5719 | N | LEU | B | 1265 | 25.824 | −1.709 | 7.791 | 1.00 | 60.48 | B |
| 5720 | CA | LEU | B | 1265 | 25.165 | −2.976 | 8.421 | 1.00 | 60.28 | B |
| 5721 | CB | LEU | B | 1265 | 23.955 | −3.519 | 7.645 | 1.00 | 59.22 | B |
| 5722 | CG | LEU | B | 1265 | 22.540 | −2.943 | 7.904 | 1.00 | 60.86 | B |
| 5723 | CD1 | LEU | B | 1265 | 21.596 | −3.668 | 6.986 | 1.00 | 63.25 | B |
| 5724 | CD2 | LEU | B | 1265 | 21.998 | −3.014 | 9.297 | 1.00 | 54.48 | B |
| 5725 | C | LEU | B | 1265 | 26.020 | −4.163 | 8.272 | 1.00 | 61.83 | B |
| 5726 | O | LEU | B | 1265 | 26.029 | −4.641 | 7.151 | 1.00 | 62.59 | B |
| 5727 | N | GLN | B | 1266 | 26.681 | −4.677 | 9.333 | 1.00 | 62.80 | B |
| 5728 | CA | GLN | B | 1266 | 27.644 | −5.815 | 9.191 | 1.00 | 61.55 | B |
| 5729 | CB | GLN | B | 1266 | 28.546 | −5.888 | 10.383 | 1.00 | 59.62 | B |
| 5730 | CG | GLN | B | 1266 | 29.874 | −6.582 | 10.166 | 1.00 | 61.70 | B |
| 5731 | CD | GLN | B | 1266 | 31.046 | −5.544 | 10.129 | 1.00 | 66.97 | B |
| 5732 | OE1 | GLN | B | 1266 | 31.199 | −4.709 | 11.055 | 1.00 | 69.42 | B |
| 5733 | NE2 | GLN | B | 1266 | 31.801 | −5.526 | 9.023 | 1.00 | 63.64 | B |
| 5734 | C | GLN | B | 1266 | 26.765 | −7.085 | 9.150 | 1.00 | 63.45 | B |
| 5735 | O | GLN | B | 1266 | 25.654 | −7.051 | 9.698 | 1.00 | 63.56 | B |
| 5736 | N | ALA | B | 1267 | 27.193 | −8.161 | 8.439 | 1.00 | 63.25 | B |
| 5737 | CA | ALA | B | 1267 | 26.512 | −9.468 | 8.505 | 1.00 | 63.41 | B |
| 5738 | CB | ALA | B | 1267 | 26.341 | −10.120 | 7.149 | 1.00 | 61.94 | B |
| 5739 | C | ALA | B | 1267 | 27.208 | −10.430 | 9.484 | 1.00 | 65.04 | B |
| 5740 | O | ALA | B | 1267 | 28.278 | −10.110 | 10.118 | 1.00 | 61.86 | B |
| 5741 | N | ALA | B | 1268 | 26.564 | −11.596 | 9.650 | 1.00 | 67.06 | B |
| 5742 | CA | ALA | B | 1268 | 27.358 | −12.748 | 10.078 | 1.00 | 70.33 | B |
| 5743 | CB | ALA | B | 1268 | 26.537 | −13.972 | 9.937 | 1.00 | 67.92 | B |
| 5744 | C | ALA | B | 1268 | 28.769 | −12.837 | 9.337 | 1.00 | 71.63 | B |
| 5745 | O | ALA | B | 1268 | 29.803 | −12.360 | 9.880 | 1.00 | 71.81 | B |
| 5746 | N | ASN | B | 1269 | 28.820 | −13.386 | 8.091 | 1.00 | 74.31 | B |
| 5747 | CA | ASN | B | 1269 | 30.144 | −13.672 | 7.399 | 1.00 | 75.37 | B |
| 5748 | CB | ASN | B | 1269 | 30.109 | −13.407 | 5.877 | 1.00 | 75.03 | B |
| 5749 | CG | ASN | B | 1269 | 30.150 | −11.815 | 5.509 | 1.00 | 78.73 | B |
| 5750 | OD1 | ASN | B | 1269 | 30.686 | −11.380 | 4.451 | 1.00 | 86.70 | B |
| 5751 | ND2 | ASN | B | 1269 | 29.582 | −10.982 | 6.381 | 1.00 | 72.30 | B |
| 5752 | C | ASN | B | 1269 | 31.010 | −12.592 | 7.901 | 1.00 | 78.47 | B |
| 5753 | O | ASN | B | 1269 | 32.208 | −12.747 | 8.440 | 1.00 | 78.18 | B |
| 5754 | N | GLU | B | 1270 | 30.411 | −11.403 | 7.543 | 1.00 | 80.00 | B |
| 5755 | CA | GLU | B | 1270 | 30.899 | −10.087 | 7.872 | 1.00 | 81.75 | B |
| 5756 | CB | GLU | B | 1270 | 32.429 | −10.175 | 8.172 | 1.00 | 84.61 | B |
| 5757 | CG | GLU | B | 1270 | 32.700 | −11.123 | 9.373 | 1.00 | 88.89 | B |
| 5758 | CD | GLU | B | 1270 | 31.781 | −10.595 | 10.406 | 1.00 | 95.74 | B |
| 5759 | OE1 | GLU | B | 1270 | 31.619 | −9.258 | 10.327 | 1.00 | 100.59 | B |
| 5760 | OE2 | GLU | B | 1270 | 31.201 | −11.445 | 11.159 | 1.00 | 98.48 | B |
| 5761 | C | GLU | B | 1270 | 30.641 | −9.291 | 6.669 | 1.00 | 80.54 | B |
| 5762 | O | GLU | B | 1270 | 30.654 | −8.220 | 5.839 | 1.00 | 84.03 | B |
| 5763 | N | ARG | B | 1271 | 31.483 | −8.652 | 7.519 | 1.00 | 77.88 | B |
| 5764 | CA | ARG | B | 1271 | 31.989 | −7.472 | 6.904 | 1.00 | 74.73 | B |
| 5765 | CB | ARG | B | 1271 | 32.711 | −7.900 | 5.523 | 1.00 | 75.80 | B |
| 5766 | CG | ARG | B | 1271 | 33.175 | −9.438 | 5.302 | 1.00 | 76.27 | B |
| 5767 | CD | ARG | B | 1271 | 34.769 | −9.628 | 5.251 | 1.00 | 78.76 | B |
| 5768 | NE | ARG | B | 1271 | 35.338 | −8.874 | 4.139 | 1.00 | 76.25 | B |
| 5769 | CZ | ARG | B | 1271 | 35.026 | −9.088 | 2.868 | 1.00 | 77.11 | B |
| 5770 | NH1 | ARG | B | 1271 | 34.157 | −10.081 | 2.494 | 1.00 | 79.02 | B |
| 5771 | NH2 | ARG | B | 1271 | 35.586 | −8.287 | 1.961 | 1.00 | 74.65 | B |
| 5772 | C | ARG | B | 1271 | 30.555 | −6.762 | 6.668 | 1.00 | 71.80 | B |
| 5773 | O | ARG | B | 1271 | 29.433 | −7.278 | 7.088 | 1.00 | 69.22 | B |
| 5774 | N | ASN | B | 1272 | 30.565 | −5.619 | 6.005 | 1.00 | 69.67 | B |
| 5775 | CA | ASN | B | 1272 | 29.276 | −4.811 | 5.944 | 1.00 | 69.77 | B |
| 5776 | CB | ASN | B | 1272 | 29.630 | −3.388 | 6.351 | 1.00 | 67.42 | B |
| 5777 | CG | ASN | B | 1272 | 31.086 | −3.242 | 6.514 | 1.00 | 64.07 | B |
| 5778 | OD1 | ASN | B | 1272 | 31.689 | −3.700 | 7.476 | 1.00 | 57.97 | B |
| 5779 | ND2 | ASN | B | 1272 | 31.684 | −2.650 | 5.524 | 1.00 | 62.13 | B |
| 5780 | C | ASN | B | 1272 | 28.450 | −4.957 | 4.607 | 1.00 | 65.32 | B |
| 5781 | O | ASN | B | 1272 | 29.029 | −5.348 | 3.657 | 1.00 | 67.35 | B |
| 5782 | N | VAL | B | 1273 | 27.125 | −4.825 | 4.538 | 1.00 | 61.88 | B |
| 5783 | CA | VAL | B | 1273 | 26.542 | −4.789 | 3.182 | 1.00 | 59.48 | B |
| 5784 | CB | VAL | B | 1273 | 25.064 | −4.694 | 3.153 | 1.00 | 58.14 | B |
| 5785 | CG1 | VAL | B | 1273 | 24.439 | −5.635 | 4.097 | 1.00 | 60.12 | B |
| 5786 | CG2 | VAL | B | 1273 | 24.726 | −3.375 | 3.608 | 1.00 | 61.27 | B |
| 5787 | C | VAL | B | 1273 | 26.995 | −3.485 | 2.509 | 1.00 | 59.21 | B |
| 5788 | O | VAL | B | 1273 | 26.943 | −2.398 | 3.149 | 1.00 | 60.51 | B |
| 5789 | N | SER | B | 1274 | 27.445 | −3.604 | 1.259 | 1.00 | 56.06 | B |
| 5790 | CA | SER | B | 1274 | 27.812 | −2.492 | 0.440 | 1.00 | 55.05 | B |
| 5791 | CB | SER | B | 1274 | 29.304 | −2.453 | 0.076 | 1.00 | 58.00 | B |
| 5792 | OG | SER | B | 1274 | 29.531 | −1.172 | −0.514 | 1.00 | 55.32 | B |
| 5793 | C | SER | B | 1274 | 27.092 | −2.517 | −0.809 | 1.00 | 52.98 | B |
| 5794 | O | SER | B | 1274 | 26.754 | −3.546 | −1.232 | 1.00 | 52.31 | B |
| 5795 | N | GLY | B | 1275 | 26.906 | −1.376 | −1.432 | 1.00 | 53.75 | B |
| 5796 | CA | GLY | B | 1275 | 26.100 | −1.304 | −2.718 | 1.00 | 55.78 | B |
| 5797 | C | GLY | B | 1275 | 27.051 | −1.037 | −3.856 | 1.00 | 56.35 | B |
| 5798 | O | GLY | B | 1275 | 26.764 | −1.366 | −4.915 | 1.00 | 57.09 | B |
| 5799 | N | ARG | B | 1276 | 28.168 | −0.385 | −3.559 | 1.00 | 60.47 | B |
| 5800 | CA | ARG | B | 1276 | 29.356 | −0.081 | −4.356 | 1.00 | 62.99 | B |
| 5801 | CB | ARG | B | 1276 | 30.646 | 0.155 | −3.483 | 1.00 | 61.78 | B |
| 5802 | CG | ARG | B | 1276 | 32.081 | −0.054 | −4.226 | 1.00 | 64.88 | B |
| 5803 | CD | ARG | B | 1276 | 33.617 | −0.057 | −3.433 | 1.00 | 63.61 | B |
| 5804 | NE | ARG | B | 1276 | 33.585 | −0.209 | −1.972 | 1.00 | 73.76 | B |
| 5805 | CZ | ARG | B | 1276 | 34.639 | −0.376 | −1.112 | 1.00 | 73.49 | B |
| 5806 | NH1 | ARG | B | 1276 | 35.941 | −0.495 | −1.583 | 1.00 | 65.99 | B |
| 5807 | NH2 | ARG | B | 1276 | 34.359 | −0.367 | 0.262 | 1.00 | 58.56 | B |
| 5808 | C | ARG | B | 1276 | 29.486 | −1.265 | −5.263 | 1.00 | 66.64 | B |
| 5809 | O | ARG | B | 1276 | 29.072 | −2.411 | −4.892 | 1.00 | 68.09 | B |
| 5810 | N | GLN | B | 1277 | 30.002 | −0.991 | −6.479 | 1.00 | 68.87 | B |
| 5811 | CA | GLN | B | 1277 | 29.961 | −1.958 | −7.541 | 1.00 | 68.86 | B |
| 5812 | CB | GLN | B | 1277 | 30.762 | −3.299 | −7.126 | 1.00 | 68.84 | B |
| 5813 | CG | GLN | B | 1277 | 32.474 | −3.241 | −7.186 | 1.00 | 71.51 | B |
| 5814 | CD | GLN | B | 1277 | 33.338 | −4.300 | −6.198 | 1.00 | 69.82 | B |
| 5815 | OE1 | GLN | B | 1277 | 34.167 | −5.077 | −6.667 | 1.00 | 58.11 | B |
| 5816 | NE2 | GLN | B | 1277 | 33.128 | −4.236 | −4.867 | 1.00 | 73.00 | B |
| 5817 | C | GLN | B | 1277 | 28.439 | −2.065 | −7.576 | 1.00 | 68.32 | B |
| 5818 | O | GLN | B | 1277 | 27.852 | −3.086 | −7.159 | 1.00 | 69.00 | B |
| 5819 | N | THR | B | 1278 | 27.711 | −1.024 | −7.948 | 1.00 | 68.13 | B |
| 5820 | CA | THR | B | 1278 | 26.206 | −1.385 | −7.851 | 1.00 | 71.38 | B |
| 5821 | CB | THR | B | 1278 | 25.166 | −0.209 | −7.549 | 1.00 | 69.54 | B |
| 5822 | OG1 | THR | B | 1278 | 23.852 | −0.781 | −7.413 | 1.00 | 71.32 | B |
| 5823 | CG2 | THR | B | 1278 | 25.160 | 0.745 | −8.586 | 1.00 | 68.49 | B |
| 5824 | C | THR | B | 1278 | 25.532 | −2.592 | −8.697 | 1.00 | 72.13 | B |
| 5825 | O | THR | B | 1278 | 26.108 | −3.135 | −9.696 | 1.00 | 72.64 | B |
| 5826 | N | MET | B | 1279 | 24.342 | −3.020 | −8.218 | 1.00 | 72.69 | B |
| 5827 | CA | MET | B | 1279 | 23.723 | −4.302 | −8.613 | 1.00 | 71.98 | B |
| 5828 | CB | MET | B | 1279 | 24.792 | −5.317 | −8.904 | 1.00 | 70.74 | B |
| 5829 | CG | MET | B | 1279 | 24.726 | −5.708 | −10.280 | 1.00 | 72.54 | B |
| 5830 | SD | MET | B | 1279 | 23.055 | −5.572 | −11.137 | 1.00 | 68.45 | B |
| 5831 | CE | MET | B | 1279 | 22.300 | −7.264 | −10.604 | 1.00 | 67.93 | B |
| 5832 | C | MET | B | 1279 | 22.796 | −5.051 | −7.666 | 1.00 | 73.09 | B |
| 5833 | O | MET | B | 1279 | 22.065 | −5.928 | −8.177 | 1.00 | 73.93 | B |
| 5834 | N | ASP | B | 1280 | 22.861 | −4.786 | −6.330 | 1.00 | 72.16 | B |
| 5835 | CA | ASP | B | 1280 | 22.240 | −5.626 | −5.349 | 1.00 | 71.16 | B |
| 5836 | CB | ASP | B | 1280 | 22.334 | −7.113 | −5.757 | 1.00 | 69.94 | B |
| 5837 | CG | ASP | B | 1280 | 22.826 | −7.985 | −4.597 | 1.00 | 65.20 | B |
| 5838 | OD1 | ASP | B | 1280 | 22.219 | −7.944 | −3.509 | 1.00 | 63.82 | B |
| 5839 | OD2 | ASP | B | 1280 | 23.867 | −8.603 | −4.718 | 1.00 | 61.36 | B |
| 5840 | C | ASP | B | 1280 | 23.037 | −5.570 | −4.038 | 1.00 | 74.06 | B |
| 5841 | O | ASP | B | 1280 | 24.316 | −5.785 | −4.044 | 1.00 | 74.60 | B |
| 5842 | N | LEU | B | 1281 | 22.273 | −5.502 | −2.905 | 1.00 | 74.36 | B |
| 5843 | CA | LEU | B | 1281 | 22.873 | −5.411 | −1.556 | 1.00 | 73.55 | B |
| 5844 | CB | LEU | B | 1281 | 21.879 | −4.847 | −0.555 | 1.00 | 72.93 | B |
| 5845 | CG | LEU | B | 1281 | 21.170 | −3.773 | −1.354 | 1.00 | 72.10 | B |
| 5846 | CD1 | LEU | B | 1281 | 19.664 | −3.663 | −1.057 | 1.00 | 75.52 | B |
| 5847 | CD2 | LEU | B | 1281 | 21.893 | −2.456 | −1.254 | 1.00 | 75.77 | B |
| 5848 | C | LEU | B | 1281 | 23.357 | −6.797 | −1.236 | 1.00 | 73.52 | B |
| 5849 | O | LEU | B | 1281 | 22.552 | −7.807 | −1.347 | 1.00 | 75.21 | B |
| 5850 | N | SER | B | 1282 | 24.652 | −6.874 | −0.927 | 1.00 | 71.46 | B |
| 5851 | CA | SER | B | 1282 | 25.285 | −8.130 | −0.803 | 1.00 | 70.56 | B |
| 5852 | CB | SER | B | 1282 | 25.774 | −8.756 | −2.196 | 1.00 | 71.93 | B |
| 5853 | OG | SER | B | 1282 | 27.064 | −9.608 | −2.151 | 1.00 | 72.51 | B |
| 5854 | C | SER | B | 1282 | 26.577 | −7.826 | −0.278 | 1.00 | 71.33 | B |
| 5855 | O | SER | B | 1282 | 26.865 | −6.966 | 0.714 | 1.00 | 68.41 | B |
| 5856 | N | ALA | B | 1283 | 27.578 | −8.420 | −1.274 | 1.00 | 72.31 | B |
| 5857 | CA | ALA | B | 1283 | 28.499 | −9.030 | −0.263 | 1.00 | 74.23 | B |
| 5858 | CB | ALA | B | 1283 | 27.812 | −10.221 | 0.662 | 1.00 | 73.01 | B |
| 5859 | C | ALA | B | 1283 | 29.856 | −9.196 | −0.345 | 1.00 | 74.27 | B |
| 5860 | O | ALA | B | 1283 | 30.373 | −10.148 | −0.988 | 1.00 | 76.21 | B |
| 5861 | N | ASN | B | 1284 | 30.311 | −8.554 | 1.063 | 1.00 | 72.80 | B |
| 5862 | CA | ASN | B | 1284 | 31.702 | −8.503 | 1.358 | 1.00 | 70.17 | B |
| 5863 | CB | ASN | B | 1284 | 32.153 | −8.924 | 0.011 | 1.00 | 69.17 | B |
| 5864 | CG | ASN | B | 1284 | 31.477 | −8.015 | −1.030 | 1.00 | 68.53 | B |
| 5865 | OD1 | ASN | B | 1284 | 30.449 | −8.320 | −1.773 | 1.00 | 71.47 | B |
| 5866 | ND2 | ASN | B | 1284 | 31.917 | −6.811 | −0.946 | 1.00 | 66.24 | B |
| 5867 | C | ASN | B | 1284 | 32.522 | −7.233 | 1.421 | 1.00 | 69.99 | B |
| 5868 | O | ASN | B | 1284 | 33.651 | −7.344 | 1.001 | 1.00 | 71.63 | B |
| 5869 | N | GLN | B | 1285 | 32.141 | −6.034 | 1.830 | 1.00 | 69.25 | B |
| 5870 | CA | GLN | B | 1285 | 33.226 | −5.001 | 1.658 | 1.00 | 69.27 | B |
| 5871 | CB | GLN | B | 1285 | 32.900 | −3.756 | 0.776 | 1.00 | 69.92 | B |
| 5872 | CG | GLN | B | 1285 | 33.241 | −3.544 | −0.793 | 1.00 | 68.29 | B |
| 5873 | CD | GLN | B | 1285 | 34.483 | −4.247 | −1.362 | 1.00 | 69.41 | B |
| 5874 | OE1 | GLN | B | 1285 | 35.570 | −3.647 | −1.594 | 1.00 | 67.67 | B |
| 5875 | NE2 | GLN | B | 1285 | 34.310 | −5.499 | −1.668 | 1.00 | 66.29 | B |
| 5876 | C | GLN | B | 1285 | 33.528 | −4.526 | 3.021 | 1.00 | 69.60 | B |
| 5877 | O | GLN | B | 1285 | 32.847 | −3.583 | 3.498 | 1.00 | 68.43 | B |
| 5878 | N | ASP | B | 1286 | 34.504 | −5.218 | 3.648 | 1.00 | 69.39 | B |
| 5879 | CA | ASP | B | 1286 | 35.271 | −4.745 | 4.792 | 1.00 | 69.12 | B |
| 5880 | CB | ASP | B | 1286 | 36.336 | −5.802 | 5.068 | 1.00 | 69.48 | B |
| 5881 | CG | ASP | B | 1286 | 37.539 | −5.725 | 4.085 | 1.00 | 74.08 | B |
| 5882 | OD1 | ASP | B | 1286 | 38.286 | −4.685 | 3.997 | 1.00 | 72.38 | B |
| 5883 | OD2 | ASP | B | 1286 | 37.757 | −6.738 | 3.365 | 1.00 | 83.49 | B |
| 5884 | C | ASP | B | 1286 | 35.879 | −3.278 | 4.668 | 1.00 | 67.68 | B |
| 5885 | O | ASP | B | 1286 | 36.698 | −2.830 | 5.459 | 1.00 | 67.35 | B |
| 5886 | N | GLU | B | 1287 | 35.452 | −2.548 | 3.651 | 1.00 | 67.59 | B |
| 5887 | CA | GLU | B | 1287 | 35.669 | −1.085 | 3.529 | 1.00 | 67.02 | B |
| 5888 | CB | GLU | B | 1287 | 35.897 | −0.718 | 2.030 | 1.00 | 69.06 | B |
| 5889 | CG | GLU | B | 1287 | 37.289 | −1.025 | 1.436 | 1.00 | 65.10 | B |
| 5890 | CD | GLU | B | 1287 | 38.236 | 0.195 | 1.539 | 1.00 | 69.46 | B |
| 5891 | OE1 | GLU | B | 1287 | 37.860 | 1.340 | 1.930 | 1.00 | 70.53 | B |
| 5892 | OE2 | GLU | B | 1287 | 39.415 | −0.004 | 1.237 | 1.00 | 71.11 | B |
| 5893 | C | GLU | B | 1287 | 34.413 | −0.387 | 4.031 | 1.00 | 65.10 | B |
| 5894 | O | GLU | B | 1287 | 33.294 | −0.728 | 3.629 | 1.00 | 64.58 | B |
| 5895 | N | GLU | B | 1288 | 34.558 | 0.544 | 4.964 | 1.00 | 65.39 | B |
| 5896 | CA | GLU | B | 1288 | 33.359 | 1.021 | 5.658 | 1.00 | 63.87 | B |
| 5897 | CB | GLU | B | 1288 | 33.455 | 0.760 | 7.152 | 1.00 | 62.79 | B |
| 5898 | CG | GLU | B | 1288 | 32.284 | 1.342 | 8.093 | 1.00 | 67.11 | B |
| 5899 | CD | GLU | B | 1288 | 32.684 | 1.883 | 9.625 | 1.00 | 63.93 | B |
| 5900 | OE1 | GLU | B | 1288 | 32.704 | 1.032 | 10.546 | 1.00 | 54.63 | B |
| 5901 | OE2 | GLU | B | 1288 | 32.906 | 3.149 | 9.857 | 1.00 | 56.11 | B |
| 5902 | C | GLU | B | 1288 | 32.939 | 2.464 | 5.198 | 1.00 | 65.31 | B |
| 5903 | O | GLU | B | 1288 | 32.602 | 3.234 | 6.113 | 1.00 | 65.23 | B |
| 5904 | N | THR | B | 1289 | 32.845 | 2.716 | 3.807 | 1.00 | 62.77 | B |
| 5905 | CA | THR | B | 1289 | 32.285 | 3.935 | 3.083 | 1.00 | 60.98 | B |
| 5906 | CB | THR | B | 1289 | 32.956 | 4.278 | 1.632 | 1.00 | 61.43 | B |
| 5907 | OG1 | THR | B | 1289 | 32.414 | 3.450 | 0.581 | 1.00 | 59.96 | B |
| 5908 | CG2 | THR | B | 1289 | 34.484 | 4.369 | 1.674 | 1.00 | 54.59 | B |
| 5909 | C | THR | B | 1289 | 30.844 | 4.362 | 2.712 | 1.00 | 61.63 | B |
| 5910 | O | THR | B | 1289 | 29.835 | 4.096 | 3.379 | 1.00 | 64.09 | B |
| 5911 | N | ASP | B | 1290 | 30.748 | 5.199 | 1.682 | 1.00 | 61.29 | B |
| 5912 | CA | ASP | B | 1290 | 29.441 | 5.841 | 1.380 | 1.00 | 59.95 | B |
| 5913 | CB | ASP | B | 1290 | 29.595 | 7.221 | 0.659 | 1.00 | 59.00 | B |
| 5914 | CG | ASP | B | 1290 | 30.179 | 8.397 | 1.691 | 1.00 | 64.98 | B |
| 5915 | OD1 | ASP | B | 1290 | 29.446 | 9.433 | 1.969 | 1.00 | 52.75 | B |
| 5916 | OD2 | ASP | B | 1290 | 31.405 | 8.236 | 2.241 | 1.00 | 65.74 | B |
| 5917 | C | ASP | B | 1290 | 28.877 | 4.608 | 0.677 | 1.00 | 58.71 | B |
| 5918 | O | ASP | B | 1290 | 28.298 | 3.761 | 1.336 | 1.00 | 62.19 | B |
| 5919 | N | GLN | B | 1291 | 29.157 | 4.303 | −0.542 | 1.00 | 55.22 | B |
| 5920 | CA | GLN | B | 1291 | 28.723 | 2.954 | −0.997 | 1.00 | 52.80 | B |
| 5921 | CB | GLN | B | 1291 | 29.702 | 2.503 | −2.098 | 1.00 | 52.14 | B |
| 5922 | CG | GLN | B | 1291 | 30.559 | 3.698 | −2.671 | 1.00 | 48.50 | B |
| 5923 | CD | GLN | B | 1291 | 32.007 | 3.163 | −2.981 | 1.00 | 60.03 | B |
| 5924 | OE1 | GLN | B | 1291 | 32.186 | 1.967 | −3.302 | 1.00 | 58.87 | B |
| 5925 | NE2 | GLN | B | 1291 | 33.057 | 4.031 | −2.799 | 1.00 | 61.18 | B |
| 5926 | C | GLN | B | 1291 | 28.135 | 1.775 | 0.057 | 1.00 | 52.95 | B |
| 5927 | O | GLN | B | 1291 | 27.086 | 1.078 | −0.178 | 1.00 | 53.82 | B |
| 5928 | N | GLU | B | 1292 | 28.752 | 1.601 | 1.202 | 1.00 | 50.12 | B |
| 5929 | CA | GLU | B | 1292 | 28.265 | 0.711 | 2.237 | 1.00 | 47.92 | B |
| 5930 | CB | GLU | B | 1292 | 29.481 | 0.276 | 2.972 | 1.00 | 47.29 | B |
| 5931 | CG | GLU | B | 1292 | 30.438 | −0.679 | 2.128 | 1.00 | 45.84 | B |
| 5932 | CD | GLU | B | 1292 | 31.293 | −0.074 | 1.096 | 1.00 | 48.41 | B |
| 5933 | OE1 | GLU | B | 1292 | 31.616 | −0.781 | 0.109 | 1.00 | 45.05 | B |
| 5934 | OE2 | GLU | B | 1292 | 31.715 | 1.118 | 1.242 | 1.00 | 57.23 | B |
| 5935 | C | GLU | B | 1292 | 27.215 | 1.371 | 3.195 | 1.00 | 49.02 | B |
| 5936 | O | GLU | B | 1292 | 26.488 | 0.643 | 3.930 | 1.00 | 48.07 | B |
| 5937 | N | THR | B | 1293 | 27.065 | 2.726 | 3.125 | 1.00 | 47.90 | B |
| 5938 | CA | THR | B | 1293 | 26.214 | 3.563 | 4.035 | 1.00 | 46.26 | B |
| 5939 | CB | THR | B | 1293 | 26.905 | 4.888 | 4.224 | 1.00 | 45.49 | B |
| 5940 | OG1 | THR | B | 1293 | 28.198 | 4.579 | 4.780 | 1.00 | 47.05 | B |
| 5941 | CG2 | THR | B | 1293 | 26.159 | 5.871 | 5.081 | 1.00 | 40.33 | B |
| 5942 | C | THR | B | 1293 | 24.780 | 3.791 | 3.548 | 1.00 | 45.12 | B |
| 5943 | O | THR | B | 1293 | 24.612 | 4.253 | 2.467 | 1.00 | 45.84 | B |
| 5944 | N | PHE | B | 1294 | 23.850 | 3.495 | 4.464 | 1.00 | 43.16 | B |
| 5945 | CA | PHE | B | 1294 | 22.440 | 3.508 | 4.470 | 1.00 | 43.49 | B |
| 5946 | CB | PHE | B | 1294 | 21.924 | 2.037 | 4.535 | 1.00 | 40.60 | B |
| 5947 | CG | PHE | B | 1294 | 22.731 | 1.156 | 3.516 | 1.00 | 47.41 | B |
| 5948 | CD1 | PHE | B | 1294 | 23.957 | 0.636 | 3.840 | 1.00 | 41.26 | B |
| 5949 | CD2 | PHE | B | 1294 | 22.363 | 1.098 | 2.173 | 1.00 | 45.04 | B |
| 5950 | CE1 | PHE | B | 1294 | 24.730 | 0.064 | 2.930 | 1.00 | 46.02 | B |
| 5951 | CE2 | PHE | B | 1294 | 23.219 | 0.517 | 1.244 | 1.00 | 46.25 | B |
| 5952 | CZ | PHE | B | 1294 | 24.408 | 0.039 | 1.615 | 1.00 | 41.13 | B |
| 5953 | C | PHE | B | 1294 | 21.852 | 4.462 | 5.563 | 1.00 | 46.04 | B |
| 5954 | O | PHE | B | 1294 | 22.353 | 4.474 | 6.768 | 1.00 | 46.02 | B |
| 5955 | N | GLN | B | 1295 | 20.824 | 5.234 | 5.050 | 1.00 | 45.82 | B |
| 5956 | CA | GLN | B | 1295 | 20.025 | 6.177 | 5.653 | 1.00 | 45.74 | B |
| 5957 | CB | GLN | B | 1295 | 19.689 | 7.216 | 4.623 | 1.00 | 46.67 | B |
| 5958 | CG | GLN | B | 1295 | 18.636 | 8.396 | 5.169 | 1.00 | 44.91 | B |
| 5959 | CD | GLN | B | 1295 | 19.040 | 9.783 | 4.667 | 1.00 | 48.38 | B |
| 5960 | OE1 | GLN | B | 1295 | 20.282 | 10.285 | 4.919 | 1.00 | 38.16 | B |
| 5961 | NE2 | GLN | B | 1295 | 18.035 | 10.436 | 3.834 | 1.00 | 42.13 | B |
| 5962 | C | GLN | B | 1295 | 18.739 | 5.430 | 5.940 | 1.00 | 49.09 | B |
| 5963 | O | GLN | B | 1295 | 17.856 | 5.224 | 5.057 | 1.00 | 51.41 | B |
| 5964 | N | LEU | B | 1296 | 18.651 | 5.011 | 7.190 | 1.00 | 47.95 | B |
| 5965 | CA | LEU | B | 1296 | 17.518 | 4.456 | 7.795 | 1.00 | 47.23 | B |
| 5966 | CB | LEU | B | 1296 | 17.905 | 4.380 | 9.237 | 1.00 | 46.42 | B |
| 5967 | CG | LEU | B | 1296 | 17.123 | 3.538 | 10.133 | 1.00 | 44.90 | B |
| 5968 | CD1 | LEU | B | 1296 | 17.584 | 2.201 | 9.543 | 1.00 | 48.45 | B |
| 5969 | CD2 | LEU | B | 1296 | 17.459 | 3.667 | 11.677 | 1.00 | 45.33 | B |
| 5970 | C | LEU | B | 1296 | 16.244 | 5.297 | 7.712 | 1.00 | 49.43 | B |
| 5971 | O | LEU | B | 1296 | 16.213 | 6.374 | 8.294 | 1.00 | 52.24 | B |
| 5972 | N | GLU | B | 1297 | 15.146 | 4.889 | 7.044 | 1.00 | 51.63 | B |
| 5973 | CA | GLU | B | 1297 | 13.960 | 5.729 | 7.289 | 1.00 | 54.01 | B |
| 5974 | CB | GLU | B | 1297 | 13.413 | 6.426 | 6.108 | 1.00 | 52.76 | B |
| 5975 | CG | GLU | B | 1297 | 14.341 | 6.390 | 4.987 | 1.00 | 56.84 | B |
| 5976 | CD | GLU | B | 1297 | 13.886 | 7.309 | 4.012 | 1.00 | 54.74 | B |
| 5977 | OE1 | GLU | B | 1297 | 12.751 | 7.013 | 3.438 | 1.00 | 54.33 | B |
| 5978 | OE2 | GLU | B | 1297 | 14.671 | 8.280 | 3.844 | 1.00 | 50.71 | B |
| 5979 | C | GLU | B | 1297 | 12.895 | 5.030 | 8.041 | 1.00 | 56.85 | B |
| 5980 | O | GLU | B | 1297 | 12.936 | 3.759 | 8.196 | 1.00 | 60.59 | B |
| 5981 | N | ILE | B | 1298 | 11.974 | 5.829 | 8.596 | 1.00 | 55.95 | B |
| 5982 | CA | ILE | B | 1298 | 10.918 | 5.258 | 9.395 | 1.00 | 54.31 | B |
| 5983 | CB | ILE | B | 1298 | 11.340 | 5.330 | 10.845 | 1.00 | 53.39 | B |
| 5984 | CG2 | ILE | B | 1298 | 10.248 | 5.171 | 11.796 | 1.00 | 53.78 | B |
| 5985 | CG1 | ILE | B | 1298 | 12.468 | 4.372 | 11.149 | 1.00 | 52.93 | B |
| 5986 | CD1 | ILE | B | 1298 | 13.786 | 5.163 | 11.164 | 1.00 | 62.50 | B |
| 5987 | C | ILE | B | 1298 | 9.633 | 6.019 | 8.925 | 1.00 | 55.86 | B |
| 5988 | O | ILE | B | 1298 | 9.707 | 7.165 | 8.546 | 1.00 | 56.54 | B |
| 5989 | N | ASP | B | 1299 | 8.519 | 5.337 | 8.741 | 1.00 | 56.21 | B |
| 5990 | CA | ASP | B | 1299 | 7.372 | 5.970 | 8.199 | 1.00 | 59.62 | B |
| 5991 | CB | ASP | B | 1299 | 6.576 | 4.937 | 7.327 | 1.00 | 61.15 | B |
| 5992 | CG | ASP | B | 1299 | 6.475 | 3.618 | 8.064 | 1.00 | 64.57 | B |
| 5993 | OD1 | ASP | B | 1299 | 7.498 | 3.339 | 8.884 | 1.00 | 61.36 | B |
| 5994 | OD2 | ASP | B | 1299 | 5.351 | 3.003 | 7.960 | 1.00 | 64.68 | B |
| 5995 | C | ASP | B | 1299 | 6.666 | 6.120 | 9.496 | 1.00 | 61.45 | B |
| 5996 | O | ASP | B | 1299 | 6.173 | 5.028 | 10.240 | 1.00 | 61.29 | B |
| 5997 | N | ARG | B | 1300 | 6.548 | 7.425 | 9.788 | 1.00 | 61.58 | B |
| 5998 | CA | ARG | B | 1300 | 6.049 | 7.831 | 11.086 | 1.00 | 62.70 | B |
| 5999 | CB | ARG | B | 1300 | 6.050 | 9.323 | 11.266 | 1.00 | 62.75 | B |
| 6000 | CG | ARG | B | 1300 | 5.042 | 10.165 | 10.577 | 1.00 | 63.88 | B |
| 6001 | CD | ARG | B | 1300 | 5.819 | 11.473 | 10.315 | 1.00 | 70.72 | B |
| 6002 | NE | ARG | B | 1300 | 5.224 | 12.293 | 9.252 | 1.00 | 69.44 | B |
| 6003 | CZ | ARG | B | 1300 | 5.856 | 13.143 | 8.470 | 1.00 | 60.52 | B |
| 6004 | NH1 | ARG | B | 1300 | 7.142 | 13.284 | 8.541 | 1.00 | 66.01 | B |
| 6005 | NH2 | ARG | B | 1300 | 5.161 | 13.834 | 7.611 | 1.00 | 63.98 | B |
| 6006 | C | ARG | B | 1300 | 4.741 | 7.191 | 11.500 | 1.00 | 64.52 | B |
| 6007 | O | ARG | B | 1300 | 4.403 | 7.307 | 12.654 | 1.00 | 65.00 | B |
| 6008 | N | ASP | B | 1301 | 4.188 | 6.293 | 10.659 | 1.00 | 66.58 | B |
| 6009 | CA | ASP | B | 1301 | 2.762 | 5.899 | 10.674 | 1.00 | 67.65 | B |
| 6010 | CB | ASP | B | 1301 | 2.116 | 6.256 | 9.298 | 1.00 | 68.45 | B |
| 6011 | CG | ASP | B | 1301 | 1.309 | 7.543 | 9.301 | 1.00 | 62.99 | B |
| 6012 | OD1 | ASP | B | 1301 | 1.880 | 8.617 | 9.066 | 1.00 | 63.76 | B |
| 6013 | OD2 | ASP | B | 1301 | 0.063 | 7.466 | 9.387 | 1.00 | 62.91 | B |
| 6014 | C | ASP | B | 1301 | 2.577 | 4.378 | 10.838 | 1.00 | 69.76 | B |
| 6015 | O | ASP | B | 1301 | 1.355 | 3.927 | 10.856 | 1.00 | 68.29 | B |
| 6016 | N | THR | B | 1302 | 3.740 | 3.622 | 10.840 | 1.00 | 68.85 | B |
| 6017 | CA | THR | B | 1302 | 3.728 | 2.153 | 10.945 | 1.00 | 66.63 | B |
| 6018 | CB | THR | B | 1302 | 3.038 | 1.568 | 9.722 | 1.00 | 69.67 | B |
| 6019 | OG1 | THR | B | 1302 | 3.372 | 2.332 | 8.500 | 1.00 | 72.61 | B |
| 6020 | CG2 | THR | B | 1302 | 1.442 | 1.439 | 9.934 | 1.00 | 67.91 | B |
| 6021 | C | THR | B | 1302 | 5.062 | 1.386 | 11.107 | 1.00 | 65.82 | B |
| 6022 | O | THR | B | 1302 | 5.020 | 0.135 | 11.108 | 1.00 | 65.31 | B |
| 6023 | N | LYS | B | 1303 | 6.218 | 2.094 | 11.203 | 1.00 | 64.75 | B |
| 6024 | CA | LYS | B | 1303 | 7.428 | 1.668 | 12.019 | 1.00 | 62.06 | B |
| 6025 | CB | LYS | B | 1303 | 6.988 | 0.966 | 13.289 | 1.00 | 60.52 | B |
| 6026 | CG | LYS | B | 1303 | 6.716 | 1.908 | 14.489 | 1.00 | 56.63 | B |
| 6027 | CD | LYS | B | 1303 | 5.196 | 2.267 | 14.503 | 1.00 | 55.51 | B |
| 6028 | CE | LYS | B | 1303 | 5.007 | 3.701 | 13.942 | 1.00 | 58.26 | B |
| 6029 | NZ | LYS | B | 1303 | 6.234 | 4.100 | 13.000 | 1.00 | 62.52 | B |
| 6030 | C | LYS | B | 1303 | 8.315 | 0.745 | 11.304 | 1.00 | 63.25 | B |
| 6031 | O | LYS | B | 1303 | 9.501 | 0.521 | 11.556 | 1.00 | 62.00 | B |
| 6032 | N | LYS | B | 1304 | 7.637 | 0.102 | 10.389 | 1.00 | 65.99 | B |
| 6033 | CA | LYS | B | 1304 | 8.285 | −0.573 | 9.292 | 1.00 | 66.27 | B |
| 6034 | CB | LYS | B | 1304 | 7.246 | −0.708 | 8.214 | 1.00 | 66.59 | B |
| 6035 | CG | LYS | B | 1304 | 6.590 | −2.034 | 8.169 | 1.00 | 65.83 | B |
| 6036 | CD | LYS | B | 1304 | 7.668 | −3.147 | 8.369 | 1.00 | 68.03 | B |
| 6037 | CE | LYS | B | 1304 | 6.917 | −4.576 | 8.385 | 1.00 | 64.38 | B |
| 6038 | NZ | LYS | B | 1304 | 6.394 | −4.900 | 9.720 | 1.00 | 50.67 | B |
| 6039 | C | LYS | B | 1304 | 9.492 | 0.296 | 8.815 | 1.00 | 66.51 | B |
| 6040 | O | LYS | B | 1304 | 9.440 | 1.586 | 8.717 | 1.00 | 62.30 | B |
| 6041 | N | CYS | B | 1305 | 10.611 | −0.437 | 8.676 | 1.00 | 67.26 | B |
| 6042 | CA | CYS | B | 1305 | 11.859 | 0.173 | 8.202 | 1.00 | 65.85 | B |
| 6043 | CB | CYS | B | 1305 | 13.027 | −0.616 | 8.635 | 1.00 | 63.20 | B |
| 6044 | SG | CYS | B | 1305 | 14.485 | 0.134 | 8.117 | 1.00 | 68.55 | B |
| 6045 | C | CYS | B | 1305 | 11.829 | 0.266 | 6.661 | 1.00 | 65.64 | B |
| 6046 | O | CYS | B | 1305 | 10.849 | −0.211 | 5.972 | 1.00 | 65.20 | B |
| 6047 | N | ALA | B | 1306 | 12.873 | 0.972 | 6.184 | 1.00 | 64.19 | B |
| 6048 | CA | ALA | B | 1306 | 13.362 | 1.022 | 4.818 | 1.00 | 61.03 | B |
| 6049 | CB | ALA | B | 1306 | 12.415 | 1.829 | 3.956 | 1.00 | 58.21 | B |
| 6050 | C | ALA | B | 1306 | 14.831 | 1.577 | 4.776 | 1.00 | 60.20 | B |
| 6051 | O | ALA | B | 1306 | 15.064 | 2.730 | 5.142 | 1.00 | 60.56 | B |
| 6052 | N | PHE | B | 1307 | 15.798 | 0.780 | 4.319 | 1.00 | 60.07 | B |
| 6053 | CA | PHE | B | 1307 | 17.130 | 1.317 | 3.932 | 1.00 | 61.29 | B |
| 6054 | CB | PHE | B | 1307 | 18.195 | 0.245 | 3.992 | 1.00 | 61.40 | B |
| 6055 | CG | PHE | B | 1307 | 18.092 | −0.659 | 5.213 | 1.00 | 63.89 | B |
| 6056 | CD1 | PHE | B | 1307 | 17.005 | −1.568 | 5.345 | 1.00 | 54.51 | B |
| 6057 | CD2 | PHE | B | 1307 | 19.071 | −0.576 | 6.243 | 1.00 | 58.95 | B |
| 6058 | CE1 | PHE | B | 1307 | 16.900 | −2.291 | 6.463 | 1.00 | 56.65 | B |
| 6059 | CE2 | PHE | B | 1307 | 18.979 | −1.396 | 7.414 | 1.00 | 60.64 | B |
| 6060 | CZ | PHE | B | 1307 | 17.942 | −2.220 | 7.546 | 1.00 | 59.31 | B |
| 6061 | C | PHE | B | 1307 | 17.258 | 2.029 | 2.520 | 1.00 | 61.72 | B |
| 6062 | O | PHE | B | 1307 | 16.811 | 1.524 | 1.515 | 1.00 | 62.99 | B |
| 6063 | N | ARG | B | 1308 | 17.909 | 3.191 | 2.534 | 1.00 | 59.31 | B |
| 6064 | CA | ARG | B | 1308 | 18.153 | 3.993 | 1.439 | 1.00 | 57.15 | B |
| 6065 | CB | ARG | B | 1308 | 17.801 | 5.423 | 1.846 | 1.00 | 58.46 | B |
| 6066 | CG | ARG | B | 1308 | 17.841 | 6.337 | 0.642 | 1.00 | 55.44 | B |
| 6067 | CD | ARG | B | 1308 | 16.759 | 7.356 | 0.726 | 1.00 | 55.96 | B |
| 6068 | NE | ARG | B | 1308 | 16.737 | 8.111 | −0.525 | 1.00 | 53.61 | B |
| 6069 | CZ | ARG | B | 1308 | 15.692 | 8.672 | −1.038 | 1.00 | 54.08 | B |
| 6070 | NH1 | ARG | B | 1308 | 14.525 | 8.689 | −0.443 | 1.00 | 57.59 | B |
| 6071 | NH2 | ARG | B | 1308 | 15.844 | 9.277 | −2.138 | 1.00 | 61.30 | B |
| 6072 | C | ARG | B | 1308 | 19.625 | 4.090 | 1.108 | 1.00 | 56.45 | B |
| 6073 | O | ARG | B | 1308 | 20.439 | 4.390 | 1.985 | 1.00 | 57.59 | B |
| 6074 | N | THR | B | 1309 | 19.978 | 3.982 | −0.174 | 1.00 | 53.61 | B |
| 6075 | CA | THR | B | 1309 | 21.424 | 3.976 | −0.540 | 1.00 | 49.79 | B |
| 6076 | CB | THR | B | 1309 | 21.718 | 3.070 | −1.711 | 1.00 | 46.51 | B |
| 6077 | OG1 | THR | B | 1309 | 21.169 | 3.707 | −2.948 | 1.00 | 55.46 | B |
| 6078 | CG2 | THR | B | 1309 | 21.076 | 1.770 | −1.491 | 1.00 | 26.74 | B |
| 6079 | C | THR | B | 1309 | 21.678 | 5.422 | −0.891 | 1.00 | 52.22 | B |
| 6080 | O | THR | B | 1309 | 20.715 | 6.175 | −1.100 | 1.00 | 50.44 | B |
| 6081 | N | HIS | B | 1310 | 22.968 | 5.769 | −0.808 | 1.00 | 55.09 | B |
| 6082 | CA | HIS | B | 1310 | 23.663 | 6.987 | −1.332 | 1.00 | 57.71 | B |
| 6083 | CB | HIS | B | 1310 | 25.182 | 6.761 | −1.192 | 1.00 | 56.85 | B |
| 6084 | CG | HIS | B | 1310 | 25.673 | 5.633 | −2.051 | 1.00 | 55.67 | B |
| 6085 | CD2 | HIS | B | 1310 | 25.037 | 4.545 | −2.519 | 1.00 | 55.09 | B |
| 6086 | ND1 | HIS | B | 1310 | 26.938 | 5.581 | −2.584 | 1.00 | 51.62 | B |
| 6087 | CE1 | HIS | B | 1310 | 27.051 | 4.518 | −3.361 | 1.00 | 42.33 | B |
| 6088 | NE2 | HIS | B | 1310 | 25.901 | 3.899 | −3.369 | 1.00 | 52.27 | B |
| 6089 | C | HIS | B | 1310 | 23.400 | 7.386 | −2.821 | 1.00 | 58.84 | B |
| 6090 | O | HIS | B | 1310 | 23.219 | 8.547 | −3.100 | 1.00 | 61.23 | B |
| 6091 | N | THR | B | 1311 | 23.362 | 6.435 | −3.725 | 1.00 | 58.38 | B |
| 6092 | CA | THR | B | 1311 | 22.934 | 6.721 | −5.058 | 1.00 | 59.83 | B |
| 6093 | CB | THR | B | 1311 | 23.411 | 5.606 | −6.204 | 1.00 | 60.47 | B |
| 6094 | OG1 | THR | B | 1311 | 22.363 | 4.687 | −6.539 | 1.00 | 68.05 | B |
| 6095 | CG2 | THR | B | 1311 | 24.757 | 4.810 | −5.803 | 1.00 | 59.16 | B |
| 6096 | C | THR | B | 1311 | 21.450 | 7.114 | −5.102 | 1.00 | 57.96 | B |
| 6097 | O | THR | B | 1311 | 20.938 | 7.436 | −6.209 | 1.00 | 56.32 | B |
| 6098 | N | GLY | B | 1312 | 20.777 | 7.108 | −3.919 | 1.00 | 56.46 | B |
| 6099 | CA | GLY | B | 1312 | 19.358 | 7.627 | −3.810 | 1.00 | 53.19 | B |
| 6100 | C | GLY | B | 1312 | 18.233 | 6.582 | −3.909 | 1.00 | 54.21 | B |
| 6101 | O | GLY | B | 1312 | 17.049 | 6.926 | −3.902 | 1.00 | 51.84 | B |
| 6102 | N | LYS | B | 1313 | 18.591 | 5.284 | −3.923 | 1.00 | 55.29 | B |
| 6103 | CA | LYS | B | 1313 | 17.671 | 4.277 | −4.412 | 1.00 | 56.06 | B |
| 6104 | CB | LYS | B | 1313 | 18.277 | 3.389 | −5.499 | 1.00 | 58.35 | B |
| 6105 | CG | LYS | B | 1313 | 18.390 | 3.915 | −6.980 | 1.00 | 50.73 | B |
| 6106 | CD | LYS | B | 1313 | 17.141 | 4.415 | −7.500 | 1.00 | 51.74 | B |
| 6107 | CE | LYS | B | 1313 | 17.410 | 5.941 | −8.232 | 1.00 | 62.24 | B |
| 6108 | NZ | LYS | B | 1313 | 18.824 | 6.311 | −8.776 | 1.00 | 56.63 | B |
| 6109 | C | LYS | B | 1313 | 17.455 | 3.412 | −3.329 | 1.00 | 58.67 | B |
| 6110 | O | LYS | B | 1313 | 18.222 | 3.439 | −2.363 | 1.00 | 61.02 | B |
| 6111 | N | TYR | B | 1314 | 16.412 | 2.594 | −3.421 | 1.00 | 59.76 | B |
| 6112 | CA | TYR | B | 1314 | 16.066 | 1.871 | −2.158 | 1.00 | 59.62 | B |
| 6113 | CB | TYR | B | 1314 | 14.621 | 2.223 | −1.802 | 1.00 | 60.86 | B |
| 6114 | CG | TYR | B | 1314 | 14.272 | 3.585 | −1.261 | 1.00 | 60.10 | B |
| 6115 | CD1 | TYR | B | 1314 | 13.665 | 4.543 | −2.072 | 1.00 | 68.28 | B |
| 6116 | CE1 | TYR | B | 1314 | 13.248 | 5.793 | −1.539 | 1.00 | 70.74 | B |
| 6117 | CD2 | TYR | B | 1314 | 14.456 | 3.875 | 0.060 | 1.00 | 62.18 | B |
| 6118 | CE2 | TYR | B | 1314 | 14.073 | 5.102 | 0.614 | 1.00 | 65.03 | B |
| 6119 | CZ | TYR | B | 1314 | 13.453 | 6.050 | −0.173 | 1.00 | 64.15 | B |
| 6120 | OH | TYR | B | 1314 | 13.089 | 7.252 | 0.372 | 1.00 | 58.23 | B |
| 6121 | C | TYR | B | 1314 | 16.054 | 0.390 | −2.244 | 1.00 | 58.39 | B |
| 6122 | O | TYR | B | 1314 | 15.649 | −0.165 | −3.246 | 1.00 | 60.02 | B |
| 6123 | N | TRP | B | 1315 | 16.386 | −0.266 | −1.161 | 1.00 | 57.78 | B |
| 6124 | CA | TRP | B | 1315 | 16.130 | −1.743 | −0.963 | 1.00 | 56.84 | B |
| 6125 | CB | TRP | B | 1315 | 16.222 | −2.097 | 0.566 | 1.00 | 53.73 | B |
| 6126 | CG | TRP | B | 1315 | 17.592 | −1.807 | 1.055 | 1.00 | 50.83 | B |
| 6127 | CD2 | TRP | B | 1315 | 18.430 | −2.589 | 1.930 | 1.00 | 39.53 | B |
| 6128 | CE2 | TRP | B | 1315 | 19.717 | −1.900 | 2.028 | 1.00 | 46.80 | B |
| 6129 | CE3 | TRP | B | 1315 | 18.242 | −3.731 | 2.643 | 1.00 | 46.39 | B |
| 6130 | CD1 | TRP | B | 1315 | 18.372 | −0.695 | 0.692 | 1.00 | 51.40 | B |
| 6131 | NE1 | TRP | B | 1315 | 19.657 | −0.766 | 1.236 | 1.00 | 50.92 | B |
| 6132 | CZ2 | TRP | B | 1315 | 20.765 | −2.332 | 2.843 | 1.00 | 51.30 | B |
| 6133 | CZ3 | TRP | B | 1315 | 19.383 | −4.222 | 3.484 | 1.00 | 55.04 | B |
| 6134 | CH2 | TRP | B | 1315 | 20.608 | −3.510 | 3.558 | 1.00 | 52.28 | B |
| 6135 | C | TRP | B | 1315 | 14.759 | −2.242 | −1.572 | 1.00 | 56.61 | B |
| 6136 | O | TRP | B | 1315 | 13.727 | −1.755 | −1.202 | 1.00 | 57.26 | B |
| 6137 | N | THR | B | 1316 | 14.735 | −3.199 | −2.469 | 1.00 | 56.17 | B |
| 6138 | CA | THR | B | 1316 | 13.479 | −3.755 | −2.848 | 1.00 | 56.93 | B |
| 6139 | CB | THR | B | 1316 | 12.829 | −3.007 | −4.219 | 1.00 | 58.26 | B |
| 6140 | OG1 | THR | B | 1316 | 11.513 | −2.458 | −3.980 | 1.00 | 49.29 | B |
| 6141 | CG2 | THR | B | 1316 | 12.835 | −3.933 | −5.527 | 1.00 | 57.58 | B |
| 6142 | C | THR | B | 1316 | 13.731 | −5.287 | −2.874 | 1.00 | 60.25 | B |
| 6143 | O | THR | B | 1316 | 14.929 | −5.852 | −3.128 | 1.00 | 61.25 | B |
| 6144 | N | LEU | B | 1317 | 12.638 | −5.998 | −2.582 | 1.00 | 59.97 | B |
| 6145 | CA | LEU | B | 1317 | 12.715 | −7.432 | −2.325 | 1.00 | 59.15 | B |
| 6146 | CB | LEU | B | 1317 | 11.549 | −7.718 | −1.442 | 1.00 | 58.33 | B |
| 6147 | CG | LEU | B | 1317 | 11.073 | −8.918 | −0.673 | 1.00 | 55.02 | B |
| 6148 | CD1 | LEU | B | 1317 | 10.132 | −9.320 | −1.695 | 1.00 | 56.53 | B |
| 6149 | CD2 | LEU | B | 1317 | 12.135 | −10.022 | −0.117 | 1.00 | 32.65 | B |
| 6150 | C | LEU | B | 1317 | 12.454 | −7.965 | −3.636 | 1.00 | 60.10 | B |
| 6151 | O | LEU | B | 1317 | 11.471 | −7.581 | −4.330 | 1.00 | 62.00 | B |
| 6152 | N | THR | B | 1318 | 13.354 | −8.794 | −4.086 | 1.00 | 60.87 | B |
| 6153 | CA | THR | B | 1318 | 13.051 | −9.314 | −5.408 | 1.00 | 61.41 | B |
| 6154 | CB | THR | B | 1318 | 14.264 | −9.514 | −6.241 | 1.00 | 59.83 | B |
| 6155 | OG1 | THR | B | 1318 | 15.082 | −10.476 | −5.588 | 1.00 | 62.57 | B |
| 6156 | CG2 | THR | B | 1318 | 15.054 | −8.215 | −6.591 | 1.00 | 53.60 | B |
| 6157 | C | THR | B | 1318 | 12.240 | −10.634 | −5.249 | 1.00 | 64.33 | B |
| 6158 | O | THR | B | 1318 | 11.177 | −10.611 | −4.519 | 1.00 | 65.34 | B |
| 6159 | N | ALA | B | 1319 | 12.735 | −11.721 | −5.880 | 1.00 | 64.73 | B |
| 6160 | CA | ALA | B | 1319 | 12.079 | −13.054 | −6.111 | 1.00 | 64.72 | B |
| 6161 | CB | ALA | B | 1319 | 11.622 | −13.311 | −7.620 | 1.00 | 63.67 | B |
| 6162 | C | ALA | B | 1319 | 13.161 | −14.062 | −5.798 | 1.00 | 66.25 | B |
| 6163 | O | ALA | B | 1319 | 12.885 | −14.891 | −4.949 | 1.00 | 65.48 | B |
| 6164 | N | THR | B | 1320 | 14.370 | −13.976 | −6.457 | 1.00 | 65.75 | B |
| 6165 | CA | THR | B | 1320 | 15.594 | −14.659 | −5.995 | 1.00 | 68.30 | B |
| 6166 | CB | THR | B | 1320 | 16.950 | −13.965 | −6.368 | 1.00 | 68.57 | B |
| 6167 | OG1 | THR | B | 1320 | 16.785 | −13.129 | −7.489 | 1.00 | 76.61 | B |
| 6168 | CG2 | THR | B | 1320 | 18.067 | −15.026 | −6.713 | 1.00 | 69.24 | B |
| 6169 | C | THR | B | 1320 | 15.726 | −14.687 | −4.455 | 1.00 | 67.88 | B |
| 6170 | O | THR | B | 1320 | 16.876 | −14.814 | −3.881 | 1.00 | 69.95 | B |
| 6171 | N | GLY | B | 1321 | 14.607 | −14.523 | −3.774 | 1.00 | 65.50 | B |
| 6172 | CA | GLY | B | 1321 | 14.653 | −13.887 | −2.472 | 1.00 | 63.65 | B |
| 6173 | C | GLY | B | 1321 | 15.746 | −12.890 | −2.217 | 1.00 | 60.50 | B |
| 6174 | O | GLY | B | 1321 | 16.169 | −12.771 | −1.050 | 1.00 | 62.39 | B |
| 6175 | N | GLY | B | 1322 | 16.182 | −12.160 | −3.285 | 1.00 | 59.07 | B |
| 6176 | CA | GLY | B | 1322 | 17.477 | −11.389 | −3.310 | 1.00 | 50.95 | B |
| 6177 | C | GLY | B | 1322 | 16.918 | −10.053 | −2.994 | 1.00 | 49.26 | B |
| 6178 | O | GLY | B | 1322 | 15.661 | −9.797 | −3.103 | 1.00 | 47.92 | B |
| 6179 | N | VAL | B | 1323 | 17.800 | −9.196 | −2.565 | 1.00 | 48.32 | B |
| 6180 | CA | VAL | B | 1323 | 17.380 | −7.865 | −2.324 | 1.00 | 49.35 | B |
| 6181 | CB | VAL | B | 1323 | 17.032 | −7.580 | −0.648 | 1.00 | 49.82 | B |
| 6182 | CG1 | VAL | B | 1323 | 16.611 | −6.024 | −0.434 | 1.00 | 46.14 | B |
| 6183 | CG2 | VAL | B | 1323 | 15.933 | −8.664 | 0.023 | 1.00 | 39.69 | B |
| 6184 | C | VAL | B | 1323 | 18.400 | −6.869 | −2.920 | 1.00 | 51.45 | B |
| 6185 | O | VAL | B | 1323 | 19.647 | −7.022 | −2.852 | 1.00 | 49.90 | B |
| 6186 | N | GLN | B | 1324 | 17.889 | −5.769 | −3.400 | 1.00 | 53.71 | B |
| 6187 | CA | GLN | B | 1324 | 18.634 | −5.135 | −4.439 | 1.00 | 58.47 | B |
| 6188 | CB | GLN | B | 1324 | 18.352 | −5.816 | −5.862 | 1.00 | 59.38 | B |
| 6189 | CG | GLN | B | 1324 | 18.963 | −7.325 | −6.077 | 1.00 | 60.37 | B |
| 6190 | CD | GLN | B | 1324 | 18.617 | −7.998 | −7.525 | 1.00 | 64.75 | B |
| 6191 | OE1 | GLN | B | 1324 | 18.748 | −9.219 | −7.647 | 1.00 | 69.06 | B |
| 6192 | NE2 | GLN | B | 1324 | 18.188 | −7.204 | −8.574 | 1.00 | 56.78 | B |
| 6193 | C | GLN | B | 1324 | 18.153 | −3.722 | −4.337 | 1.00 | 57.91 | B |
| 6194 | O | GLN | B | 1324 | 17.014 | −3.439 | −3.797 | 1.00 | 60.51 | B |
| 6195 | N | SER | B | 1325 | 18.953 | −2.779 | −4.820 | 1.00 | 55.94 | B |
| 6196 | CA | SER | B | 1325 | 18.582 | −1.444 | −4.355 | 1.00 | 54.92 | B |
| 6197 | CB | SER | B | 1325 | 19.734 | −0.834 | −3.635 | 1.00 | 52.57 | B |
| 6198 | OG | SER | B | 1325 | 20.516 | −0.134 | −4.457 | 1.00 | 45.46 | B |
| 6199 | C | SER | B | 1325 | 18.009 | −0.607 | −5.434 | 1.00 | 55.72 | B |
| 6200 | O | SER | B | 1325 | 18.707 | 0.233 | −6.032 | 1.00 | 56.08 | B |
| 6201 | N | THR | B | 1326 | 16.740 | −0.824 | −5.698 | 1.00 | 56.01 | B |
| 6202 | CA | THR | B | 1326 | 16.234 | −0.295 | −6.911 | 1.00 | 58.59 | B |
| 6203 | CB | THR | B | 1326 | 15.908 | −1.416 | −7.673 | 1.00 | 57.91 | B |
| 6204 | OG1 | THR | B | 1326 | 17.087 | −1.582 | −8.480 | 1.00 | 63.81 | B |
| 6205 | CG2 | THR | B | 1326 | 14.519 | −1.239 | −8.507 | 1.00 | 58.53 | B |
| 6206 | C | THR | B | 1326 | 15.144 | 0.725 | −6.952 | 1.00 | 60.15 | B |
| 6207 | O | THR | B | 1326 | 15.232 | 1.758 | −7.657 | 1.00 | 62.95 | B |
| 6208 | N | ALA | B | 1327 | 14.055 | 0.393 | −6.317 | 1.00 | 62.23 | B |
| 6209 | CA | ALA | B | 1327 | 13.028 | 1.367 | −6.075 | 1.00 | 64.94 | B |
| 6210 | CB | ALA | B | 1327 | 12.194 | 1.003 | −4.722 | 1.00 | 64.67 | B |
| 6211 | C | ALA | B | 1327 | 13.612 | 2.785 | −5.961 | 1.00 | 64.80 | B |
| 6212 | O | ALA | B | 1327 | 14.447 | 3.095 | −5.118 | 1.00 | 65.82 | B |
| 6213 | N | SER | B | 1328 | 13.146 | 3.571 | −6.868 | 1.00 | 65.40 | B |
| 6214 | CA | SER | B | 1328 | 13.283 | 4.963 | −6.948 | 1.00 | 69.15 | B |
| 6215 | CB | SER | B | 1328 | 13.036 | 5.312 | −8.434 | 1.00 | 69.00 | B |
| 6216 | OG | SER | B | 1328 | 13.732 | 4.342 | −9.312 | 1.00 | 79.01 | B |
| 6217 | C | SER | B | 1328 | 12.224 | 5.679 | −6.049 | 1.00 | 69.31 | B |
| 6218 | O | SER | B | 1328 | 12.528 | 6.732 | −5.443 | 1.00 | 70.51 | B |
| 6219 | N | SER | B | 1329 | 10.967 | 5.165 | −6.027 | 1.00 | 69.53 | B |
| 6220 | CA | SER | B | 1329 | 9.948 | 5.547 | −5.015 | 1.00 | 67.45 | B |
| 6221 | CB | SER | B | 1329 | 8.572 | 5.833 | −5.630 | 1.00 | 68.11 | B |
| 6222 | OG | SER | B | 1329 | 7.963 | 6.925 | −4.919 | 1.00 | 64.39 | B |
| 6223 | C | SER | B | 1329 | 9.850 | 4.643 | −3.730 | 1.00 | 66.29 | B |
| 6224 | O | SER | B | 1329 | 10.541 | 3.708 | −3.546 | 1.00 | 66.02 | B |
| 6225 | N | LYS | B | 1330 | 9.026 | 5.059 | −2.819 | 1.00 | 65.44 | B |
| 6226 | CA | LYS | B | 1330 | 8.741 | 4.423 | −1.563 | 1.00 | 65.66 | B |
| 6227 | CB | LYS | B | 1330 | 8.179 | 5.569 | −0.712 | 1.00 | 66.32 | B |
| 6228 | CG | LYS | B | 1330 | 8.609 | 7.061 | −1.427 | 1.00 | 67.86 | B |
| 6229 | CD | LYS | B | 1330 | 8.126 | 8.342 | −0.690 | 1.00 | 67.00 | B |
| 6230 | CE | LYS | B | 1330 | 7.745 | 8.078 | 0.801 | 1.00 | 63.76 | B |
| 6231 | NZ | LYS | B | 1330 | 6.276 | 8.412 | 1.169 | 1.00 | 63.23 | B |
| 6232 | C | LYS | B | 1330 | 7.740 | 3.232 | −1.741 | 1.00 | 65.41 | B |
| 6233 | O | LYS | B | 1330 | 6.771 | 3.021 | −0.956 | 1.00 | 64.30 | B |
| 6234 | N | ASN | B | 1331 | 7.943 | 2.437 | −2.774 | 1.00 | 64.27 | B |
| 6235 | CA | ASN | B | 1331 | 7.248 | 1.130 | −2.722 | 1.00 | 65.65 | B |
| 6236 | CB | ASN | B | 1331 | 7.570 | 0.156 | −3.974 | 1.00 | 65.80 | B |
| 6237 | CG | ASN | B | 1331 | 7.476 | −1.357 | −3.606 | 1.00 | 63.16 | B |
| 6238 | OD1 | ASN | B | 1331 | 8.273 | −2.169 | −4.005 | 1.00 | 57.47 | B |
| 6239 | ND2 | ASN | B | 1331 | 6.524 | −1.680 | −2.786 | 1.00 | 64.91 | B |
| 6240 | C | ASN | B | 1331 | 7.278 | 0.416 | −1.284 | 1.00 | 64.30 | B |
| 6241 | O | ASN | B | 1331 | 8.309 | 0.258 | −0.625 | 1.00 | 62.23 | B |
| 6242 | N | ALA | B | 1332 | 6.097 | −0.049 | −0.904 | 1.00 | 63.96 | B |
| 6243 | CA | ALA | B | 1332 | 5.892 | −0.814 | 0.262 | 1.00 | 65.17 | B |
| 6244 | CB | ALA | B | 1332 | 4.451 | −0.774 | 0.663 | 1.00 | 66.91 | B |
| 6245 | C | ALA | B | 1332 | 6.378 | −2.236 | 0.020 | 1.00 | 65.70 | B |
| 6246 | O | ALA | B | 1332 | 6.073 | −3.170 | 0.779 | 1.00 | 65.59 | B |
| 6247 | N | SER | B | 1333 | 7.281 | −2.331 | −0.957 | 1.00 | 65.47 | B |
| 6248 | CA | SER | B | 1333 | 8.079 | −3.532 | −1.254 | 1.00 | 65.65 | B |
| 6249 | CB | SER | B | 1333 | 8.324 | −3.504 | −2.760 | 1.00 | 65.16 | B |
| 6250 | OG | SER | B | 1333 | 8.202 | −4.795 | −3.330 | 1.00 | 71.97 | B |
| 6251 | C | SER | B | 1333 | 9.409 | −3.430 | −0.534 | 1.00 | 63.68 | B |
| 6252 | O | SER | B | 1333 | 10.250 | −4.432 | −0.440 | 1.00 | 60.52 | B |
| 6253 | N | CYS | B | 1334 | 9.608 | −2.180 | −0.068 | 1.00 | 62.46 | B |
| 6254 | CA | CYS | B | 1334 | 10.952 | −1.785 | 0.365 | 1.00 | 62.56 | B |
| 6255 | CB | CYS | B | 1334 | 11.339 | −0.479 | −0.251 | 1.00 | 61.17 | B |
| 6256 | SG | CYS | B | 1334 | 10.595 | −0.216 | −1.812 | 1.00 | 62.57 | B |
| 6257 | C | CYS | B | 1334 | 11.084 | −1.670 | 1.899 | 1.00 | 62.62 | B |
| 6258 | O | CYS | B | 1334 | 12.161 | −1.224 | 2.395 | 1.00 | 60.98 | B |
| 6259 | N | TYR | B | 1335 | 9.989 | −2.088 | 2.580 | 1.00 | 61.56 | B |
| 6260 | CA | TYR | B | 1335 | 9.715 | −1.854 | 3.972 | 1.00 | 61.58 | B |
| 6261 | CB | TYR | B | 1335 | 8.296 | −1.364 | 4.075 | 1.00 | 62.45 | B |
| 6262 | CG | TYR | B | 1335 | 8.156 | 0.125 | 3.947 | 1.00 | 67.38 | B |
| 6263 | CD1 | TYR | B | 1335 | 7.882 | 0.732 | 2.714 | 1.00 | 66.79 | B |
| 6264 | CE1 | TYR | B | 1335 | 7.685 | 2.070 | 2.624 | 1.00 | 63.09 | B |
| 6265 | CD2 | TYR | B | 1335 | 8.224 | 0.960 | 5.084 | 1.00 | 69.90 | B |
| 6266 | CE2 | TYR | B | 1335 | 8.070 | 2.363 | 4.969 | 1.00 | 66.60 | B |
| 6267 | CZ | TYR | B | 1335 | 7.826 | 2.887 | 3.746 | 1.00 | 66.48 | B |
| 6268 | OH | TYR | B | 1335 | 7.648 | 4.283 | 3.672 | 1.00 | 72.75 | B |
| 6269 | C | TYR | B | 1335 | 9.945 | −3.108 | 4.830 | 1.00 | 60.91 | B |
| 6270 | O | TYR | B | 1335 | 9.077 | −3.969 | 5.035 | 1.00 | 59.64 | B |
| 6271 | N | PHE | B | 1336 | 11.138 | −3.186 | 5.365 | 1.00 | 62.32 | B |
| 6272 | CA | PHE | B | 1336 | 11.545 | −4.284 | 6.202 | 1.00 | 63.37 | B |
| 6273 | CB | PHE | B | 1336 | 13.021 | −4.484 | 5.980 | 1.00 | 64.69 | B |
| 6274 | CG | PHE | B | 1336 | 13.303 | −5.173 | 4.639 | 1.00 | 68.16 | B |
| 6275 | CD1 | PHE | B | 1336 | 14.462 | −5.858 | 4.414 | 1.00 | 69.57 | B |
| 6276 | CD2 | PHE | B | 1336 | 12.295 | −5.211 | 3.616 | 1.00 | 73.01 | B |
| 6277 | CE1 | PHE | B | 1336 | 14.661 | −6.520 | 3.194 | 1.00 | 70.99 | B |
| 6278 | CE2 | PHE | B | 1336 | 12.442 | −5.943 | 2.382 | 1.00 | 68.68 | B |
| 6279 | CZ | PHE | B | 1336 | 13.625 | −6.574 | 2.160 | 1.00 | 69.34 | B |
| 6280 | C | PHE | B | 1336 | 11.127 | −4.034 | 7.627 | 1.00 | 64.54 | B |
| 6281 | O | PHE | B | 1336 | 11.450 | −3.032 | 8.311 | 1.00 | 64.17 | B |
| 6282 | N | ASP | B | 1337 | 10.309 | −4.940 | 8.094 | 1.00 | 64.71 | B |
| 6283 | CA | ASP | B | 1337 | 10.291 | −5.065 | 9.585 | 1.00 | 64.08 | B |
| 6284 | CB | ASP | B | 1337 | 9.868 | −6.461 | 9.935 | 1.00 | 64.52 | B |
| 6285 | CG | ASP | B | 1337 | 8.954 | −6.402 | 10.883 | 1.00 | 67.54 | B |
| 6286 | OD1 | ASP | B | 1337 | 8.836 | −7.457 | 11.647 | 1.00 | 71.17 | B |
| 6287 | OD2 | ASP | B | 1337 | 8.471 | −5.167 | 10.856 | 1.00 | 61.75 | B |
| 6288 | C | ASP | B | 1337 | 11.639 | −4.958 | 10.300 | 1.00 | 60.74 | B |
| 6289 | O | ASP | B | 1337 | 12.470 | −5.869 | 10.113 | 1.00 | 56.39 | B |
| 6290 | N | ILE | B | 1338 | 11.820 | −3.995 | 11.199 | 1.00 | 59.42 | B |
| 6291 | CA | ILE | B | 1338 | 12.913 | −4.299 | 12.192 | 1.00 | 60.22 | B |
| 6292 | CB | ILE | B | 1338 | 13.885 | −3.163 | 12.459 | 1.00 | 58.76 | B |
| 6293 | CG2 | ILE | B | 1338 | 14.879 | −3.759 | 13.212 | 1.00 | 58.20 | B |
| 6294 | CG1 | ILE | B | 1338 | 14.628 | −2.712 | 11.179 | 1.00 | 58.40 | B |
| 6295 | CD1 | ILE | B | 1338 | 15.136 | −1.195 | 11.182 | 1.00 | 57.91 | B |
| 6296 | C | ILE | B | 1338 | 12.498 | −5.012 | 13.527 | 1.00 | 61.22 | B |
| 6297 | O | ILE | B | 1338 | 11.467 | −4.668 | 14.141 | 1.00 | 61.21 | B |
| 6298 | N | GLU | B | 1339 | 13.227 | −6.057 | 13.956 | 1.00 | 61.41 | B |
| 6299 | CA | GLU | B | 1339 | 13.063 | −6.493 | 15.415 | 1.00 | 59.86 | B |
| 6300 | CB | GLU | B | 1339 | 12.677 | −7.964 | 15.657 | 1.00 | 58.57 | B |
| 6301 | CG | GLU | B | 1339 | 13.417 | −8.693 | 16.932 | 1.00 | 59.01 | B |
| 6302 | CD | GLU | B | 1339 | 14.260 | −10.010 | 16.620 | 1.00 | 58.34 | B |
| 6303 | OE1 | GLU | B | 1339 | 13.736 | −10.888 | 15.901 | 1.00 | 59.31 | B |
| 6304 | OE2 | GLU | B | 1339 | 15.434 | −10.134 | 17.051 | 1.00 | 57.05 | B |
| 6305 | C | GLU | B | 1339 | 14.342 | −6.117 | 16.148 | 1.00 | 60.00 | B |
| 6306 | O | GLU | B | 1339 | 15.431 | −6.312 | 15.594 | 1.00 | 59.83 | B |
| 6307 | N | TRP | B | 1340 | 14.187 | −5.583 | 17.381 | 1.00 | 61.54 | B |
| 6308 | CA | TRP | B | 1340 | 15.214 | −4.920 | 18.177 | 1.00 | 60.26 | B |
| 6309 | CB | TRP | B | 1340 | 14.519 | −3.817 | 18.954 | 1.00 | 58.16 | B |
| 6310 | CG | TRP | B | 1340 | 14.188 | −2.662 | 17.948 | 1.00 | 56.03 | B |
| 6311 | CD2 | TRP | B | 1340 | 15.099 | −1.945 | 17.095 | 1.00 | 52.75 | B |
| 6312 | CE2 | TRP | B | 1340 | 14.340 | −1.044 | 16.312 | 1.00 | 56.59 | B |
| 6313 | CE3 | TRP | B | 1340 | 16.499 | −1.868 | 17.013 | 1.00 | 56.23 | B |
| 6314 | CD1 | TRP | B | 1340 | 12.925 | −2.240 | 17.563 | 1.00 | 56.95 | B |
| 6315 | NE1 | TRP | B | 1340 | 13.011 | −1.239 | 16.604 | 1.00 | 50.79 | B |
| 6316 | CZ2 | TRP | B | 1340 | 14.970 | −0.085 | 15.377 | 1.00 | 60.02 | B |
| 6317 | CZ3 | TRP | B | 1340 | 17.136 | −0.906 | 16.125 | 1.00 | 52.28 | B |
| 6318 | CH2 | TRP | B | 1340 | 16.359 | −0.054 | 15.314 | 1.00 | 56.05 | B |
| 6319 | C | TRP | B | 1340 | 15.826 | −5.956 | 19.014 | 1.00 | 61.27 | B |
| 6320 | O | TRP | B | 1340 | 15.278 | −6.284 | 20.024 | 1.00 | 61.18 | B |
| 6321 | N | ARG | B | 1341 | 16.909 | −6.571 | 18.517 | 1.00 | 63.30 | B |
| 6322 | CA | ARG | B | 1341 | 17.341 | −7.940 | 19.055 | 1.00 | 63.38 | B |
| 6323 | CB | ARG | B | 1341 | 17.455 | −9.043 | 17.915 | 1.00 | 64.35 | B |
| 6324 | CG | ARG | B | 1341 | 17.546 | −10.589 | 18.270 | 1.00 | 68.12 | B |
| 6325 | CD | ARG | B | 1341 | 18.626 | −11.047 | 19.473 | 1.00 | 75.04 | B |
| 6326 | NE | ARG | B | 1341 | 19.461 | −12.228 | 19.199 | 1.00 | 69.85 | B |
| 6327 | CZ | ARG | B | 1341 | 20.741 | −12.327 | 19.569 | 1.00 | 70.89 | B |
| 6328 | NH1 | ARG | B | 1341 | 21.285 | −11.324 | 20.281 | 1.00 | 65.96 | B |
| 6329 | NH2 | ARG | B | 1341 | 21.473 | −13.444 | 19.256 | 1.00 | 62.21 | B |
| 6330 | C | ARG | B | 1341 | 18.622 | −7.540 | 19.827 | 1.00 | 61.16 | B |
| 6331 | O | ARG | B | 1341 | 19.472 | −8.269 | 20.203 | 1.00 | 56.86 | B |
| 6332 | N | ASP | B | 1342 | 18.617 | −6.260 | 20.080 | 1.00 | 62.48 | B |
| 6333 | CA | ASP | B | 1342 | 19.538 | −5.551 | 21.035 | 1.00 | 61.62 | B |
| 6334 | CB | ASP | B | 1342 | 19.153 | −5.826 | 22.423 | 1.00 | 61.06 | B |
| 6335 | CG | ASP | B | 1342 | 17.816 | −5.424 | 22.623 | 1.00 | 64.82 | B |
| 6336 | OD1 | ASP | B | 1342 | 17.498 | −4.388 | 21.951 | 1.00 | 64.19 | B |
| 6337 | OD2 | ASP | B | 1342 | 17.089 | −6.133 | 23.392 | 1.00 | 68.92 | B |
| 6338 | C | ASP | B | 1342 | 20.984 | −5.783 | 20.868 | 1.00 | 59.96 | B |
| 6339 | O | ASP | B | 1342 | 21.460 | −6.749 | 21.485 | 1.00 | 59.52 | B |
| 6340 | N | ARG | B | 1343 | 21.622 | −4.973 | 19.998 | 1.00 | 55.88 | B |
| 6341 | CA | ARG | B | 1343 | 23.001 | −5.222 | 19.594 | 1.00 | 54.99 | B |
| 6342 | CB | ARG | B | 1343 | 23.769 | −5.671 | 20.822 | 1.00 | 55.95 | B |
| 6343 | CG | ARG | B | 1343 | 25.225 | −5.916 | 20.750 | 1.00 | 56.80 | B |
| 6344 | CD | ARG | B | 1343 | 25.361 | −7.082 | 21.772 | 1.00 | 65.03 | B |
| 6345 | NE | ARG | B | 1343 | 25.314 | −6.764 | 23.197 | 1.00 | 59.82 | B |
| 6346 | CZ | ARG | B | 1343 | 26.245 | −6.043 | 23.726 | 1.00 | 61.56 | B |
| 6347 | NH1 | ARG | B | 1343 | 27.202 | −5.571 | 22.969 | 1.00 | 66.89 | B |
| 6348 | NH2 | ARG | B | 1343 | 26.272 | −5.834 | 24.991 | 1.00 | 66.47 | B |
| 6349 | C | ARG | B | 1343 | 23.243 | −6.151 | 18.439 | 1.00 | 54.47 | B |
| 6350 | O | ARG | B | 1343 | 24.370 | −6.580 | 18.180 | 1.00 | 56.15 | B |
| 6351 | N | ARG | B | 1344 | 22.184 | −6.512 | 17.756 | 1.00 | 53.53 | B |
| 6352 | CA | ARG | B | 1344 | 22.224 | −7.300 | 16.552 | 1.00 | 52.75 | B |
| 6353 | CB | ARG | B | 1344 | 22.961 | −8.652 | 16.661 | 1.00 | 52.87 | B |
| 6354 | CG | ARG | B | 1344 | 24.518 | −8.518 | 16.251 | 1.00 | 53.39 | B |
| 6355 | CD | ARG | B | 1344 | 25.638 | −8.893 | 17.381 | 1.00 | 55.76 | B |
| 6356 | NE | ARG | B | 1344 | 27.020 | −8.390 | 17.094 | 1.00 | 54.08 | B |
| 6357 | CZ | ARG | B | 1344 | 27.943 | −8.150 | 18.037 | 1.00 | 50.24 | B |
| 6358 | NH1 | ARG | B | 1344 | 29.113 | −7.570 | 17.751 | 1.00 | 30.40 | B |
| 6359 | NH2 | ARG | B | 1344 | 27.701 | −8.476 | 19.314 | 1.00 | 58.72 | B |
| 6360 | C | ARG | B | 1344 | 20.812 | −7.380 | 16.474 | 1.00 | 52.45 | B |
| 6361 | O | ARG | B | 1344 | 20.104 | −7.160 | 17.512 | 1.00 | 55.12 | B |
| 6362 | N | ILE | B | 1345 | 20.362 | −7.537 | 15.248 | 1.00 | 53.22 | B |
| 6363 | CA | ILE | B | 1345 | 19.056 | −7.037 | 14.789 | 1.00 | 52.19 | B |
| 6364 | CB | ILE | B | 1345 | 19.254 | −5.573 | 14.411 | 1.00 | 53.37 | B |
| 6365 | CG2 | ILE | B | 1345 | 18.018 | −5.026 | 13.626 | 1.00 | 54.05 | B |
| 6366 | CG1 | ILE | B | 1345 | 19.076 | −4.672 | 15.656 | 1.00 | 55.15 | B |
| 6367 | CD1 | ILE | B | 1345 | 20.265 | −3.784 | 16.406 | 1.00 | 48.51 | B |
| 6368 | C | ILE | B | 1345 | 18.506 | −7.863 | 13.560 | 1.00 | 52.77 | B |
| 6369 | O | ILE | B | 1345 | 19.241 | −8.605 | 12.860 | 1.00 | 49.66 | B |
| 6370 | N | THR | B | 1346 | 17.214 | −7.797 | 13.274 | 1.00 | 53.63 | B |
| 6371 | CA | THR | B | 1346 | 16.761 | −8.918 | 12.405 | 1.00 | 55.23 | B |
| 6372 | CB | THR | B | 1346 | 15.876 | −10.114 | 13.126 | 1.00 | 57.65 | B |
| 6373 | OG1 | THR | B | 1346 | 16.437 | −10.580 | 14.401 | 1.00 | 57.45 | B |
| 6374 | CG2 | THR | B | 1346 | 15.751 | −11.271 | 12.172 | 1.00 | 55.04 | B |
| 6375 | C | THR | B | 1346 | 15.856 | −8.335 | 11.471 | 1.00 | 55.72 | B |
| 6376 | O | THR | B | 1346 | 14.718 | −7.745 | 11.856 | 1.00 | 55.09 | B |
| 6377 | N | LEU | B | 1347 | 16.333 | −8.461 | 10.237 | 1.00 | 55.36 | B |
| 6378 | CA | LEU | B | 1347 | 15.515 | −7.878 | 9.238 | 1.00 | 54.29 | B |
| 6379 | CB | LEU | B | 1347 | 16.342 | −7.183 | 8.218 | 1.00 | 55.56 | B |
| 6380 | CG | LEU | B | 1347 | 17.099 | −5.898 | 8.247 | 1.00 | 56.22 | B |
| 6381 | CD1 | LEU | B | 1347 | 17.993 | −5.910 | 9.523 | 1.00 | 51.61 | B |
| 6382 | CD2 | LEU | B | 1347 | 17.929 | −5.948 | 6.799 | 1.00 | 52.01 | B |
| 6383 | C | LEU | B | 1347 | 14.593 | −9.022 | 8.697 | 1.00 | 52.51 | B |
| 6384 | O | LEU | B | 1347 | 15.059 | −10.115 | 8.731 | 1.00 | 53.13 | B |
| 6385 | N | ARG | B | 1348 | 13.413 | −8.681 | 8.183 | 1.00 | 48.76 | B |
| 6386 | CA | ARG | B | 1348 | 12.261 | −9.417 | 8.079 | 1.00 | 51.51 | B |
| 6387 | CB | ARG | B | 1348 | 11.570 | −9.674 | 9.498 | 1.00 | 55.09 | B |
| 6388 | CG | ARG | B | 1348 | 11.913 | −11.228 | 10.343 | 1.00 | 54.89 | B |
| 6389 | CD | ARG | B | 1348 | 10.619 | −12.100 | 10.849 | 1.00 | 49.11 | B |
| 6390 | NE | ARG | B | 1348 | 9.520 | −11.080 | 11.107 | 1.00 | 64.61 | B |
| 6391 | CZ | ARG | B | 1348 | 8.780 | −10.781 | 12.232 | 1.00 | 60.68 | B |
| 6392 | NH1 | ARG | B | 1348 | 8.891 | −11.468 | 13.352 | 1.00 | 65.67 | B |
| 6393 | NH2 | ARG | B | 1348 | 7.889 | −9.748 | 12.258 | 1.00 | 55.16 | B |
| 6394 | C | ARG | B | 1348 | 11.291 | −8.792 | 6.960 | 1.00 | 53.82 | B |
| 6395 | O | ARG | B | 1348 | 10.413 | −7.777 | 7.056 | 1.00 | 51.71 | B |
| 6396 | N | ALA | B | 1349 | 11.529 | −9.440 | 5.831 | 1.00 | 54.47 | B |
| 6397 | CA | ALA | B | 1349 | 11.299 | −8.900 | 4.552 | 1.00 | 55.85 | B |
| 6398 | CB | ALA | B | 1349 | 11.976 | −9.763 | 3.583 | 1.00 | 54.99 | B |
| 6399 | C | ALA | B | 1349 | 9.811 | −8.858 | 4.381 | 1.00 | 57.33 | B |
| 6400 | O | ALA | B | 1349 | 9.122 | −9.186 | 5.301 | 1.00 | 56.70 | B |
| 6401 | N | SER | B | 1350 | 9.368 | −8.337 | 3.243 | 1.00 | 60.28 | B |
| 6402 | CA | SER | B | 1350 | 8.076 | −8.609 | 2.564 | 1.00 | 61.18 | B |
| 6403 | CB | SER | B | 1350 | 8.037 | −7.843 | 1.150 | 1.00 | 62.71 | B |
| 6404 | OG | SER | B | 1350 | 8.564 | −6.423 | 0.971 | 1.00 | 59.40 | B |
| 6405 | C | SER | B | 1350 | 7.779 | −10.194 | 2.361 | 1.00 | 62.39 | B |
| 6406 | O | SER | B | 1350 | 6.632 | −10.643 | 1.993 | 1.00 | 64.67 | B |
| 6407 | N | ASN | B | 1351 | 8.791 | −11.016 | 2.624 | 1.00 | 60.61 | B |
| 6408 | CA | ASN | B | 1351 | 8.765 | −12.522 | 2.482 | 1.00 | 59.12 | B |
| 6409 | CB | ASN | B | 1351 | 9.716 | −12.871 | 1.321 | 1.00 | 58.24 | B |
| 6410 | CG | ASN | B | 1351 | 11.273 | −12.628 | 1.682 | 1.00 | 61.23 | B |
| 6411 | OD1 | ASN | B | 1351 | 11.687 | −11.476 | 1.961 | 1.00 | 61.99 | B |
| 6412 | ND2 | ASN | B | 1351 | 12.141 | −13.709 | 1.590 | 1.00 | 61.26 | B |
| 6413 | C | ASN | B | 1351 | 9.074 | −13.571 | 3.762 | 1.00 | 57.41 | B |
| 6414 | O | ASN | B | 1351 | 9.458 | −14.741 | 3.520 | 1.00 | 55.30 | B |
| 6415 | N | GLY | B | 1352 | 8.791 | −13.184 | 5.024 | 1.00 | 56.17 | B |
| 6416 | CA | GLY | B | 1352 | 9.381 | −13.768 | 6.274 | 1.00 | 53.52 | B |
| 6417 | C | GLY | B | 1352 | 10.911 | −13.752 | 6.392 | 1.00 | 52.25 | B |
| 6418 | O | GLY | B | 1352 | 11.666 | −12.750 | 6.477 | 1.00 | 48.19 | B |
| 6419 | N | LYS | B | 1353 | 11.366 | −14.982 | 6.448 | 1.00 | 54.18 | B |
| 6420 | CA | LYS | B | 1353 | 12.720 | −15.360 | 6.677 | 1.00 | 54.61 | B |
| 6421 | CB | LYS | B | 1353 | 13.192 | −16.353 | 5.599 | 1.00 | 54.63 | B |
| 6422 | CG | LYS | B | 1353 | 12.791 | −16.150 | 4.309 | 1.00 | 51.56 | B |
| 6423 | CD | LYS | B | 1353 | 12.410 | −17.486 | 3.732 | 1.00 | 55.63 | B |
| 6424 | CE | LYS | B | 1353 | 11.204 | −17.299 | 2.606 | 1.00 | 56.61 | B |
| 6425 | NZ | LYS | B | 1353 | 9.616 | −17.055 | 3.045 | 1.00 | 49.42 | B |
| 6426 | C | LYS | B | 1353 | 13.721 | −14.206 | 6.840 | 1.00 | 57.90 | B |
| 6427 | O | LYS | B | 1353 | 13.548 | −12.989 | 6.389 | 1.00 | 53.36 | B |
| 6428 | N | PHE | B | 1354 | 14.774 | −14.640 | 7.565 | 1.00 | 60.54 | B |
| 6429 | CA | PHE | B | 1354 | 15.839 | −13.729 | 8.096 | 1.00 | 62.38 | B |
| 6430 | CB | PHE | B | 1354 | 16.428 | −14.241 | 9.402 | 1.00 | 61.68 | B |
| 6431 | CG | PHE | B | 1354 | 15.390 | −14.689 | 10.399 | 1.00 | 62.33 | B |
| 6432 | CD1 | PHE | B | 1354 | 14.453 | −13.820 | 10.903 | 1.00 | 67.23 | B |
| 6433 | CD2 | PHE | B | 1354 | 15.372 | −15.982 | 10.852 | 1.00 | 57.79 | B |
| 6434 | CE1 | PHE | B | 1354 | 13.495 | −14.279 | 11.797 | 1.00 | 68.78 | B |
| 6435 | CE2 | PHE | B | 1354 | 14.479 | −16.407 | 11.701 | 1.00 | 52.22 | B |
| 6436 | CZ | PHE | B | 1354 | 13.526 | −15.569 | 12.177 | 1.00 | 63.72 | B |
| 6437 | C | PHE | B | 1354 | 16.904 | −13.345 | 7.091 | 1.00 | 63.46 | B |
| 6438 | O | PHE | B | 1354 | 17.840 | −14.029 | 6.696 | 1.00 | 63.89 | B |
| 6439 | N | VAL | B | 1355 | 16.702 | −12.216 | 6.557 | 1.00 | 65.02 | B |
| 6440 | CA | VAL | B | 1355 | 17.783 | −11.646 | 5.848 | 1.00 | 64.85 | B |
| 6441 | CB | VAL | B | 1355 | 17.453 | −10.088 | 5.882 | 1.00 | 66.01 | B |
| 6442 | CG1 | VAL | B | 1355 | 18.601 | −9.236 | 5.355 | 1.00 | 62.57 | B |
| 6443 | CG2 | VAL | B | 1355 | 15.844 | −9.822 | 5.352 | 1.00 | 54.98 | B |
| 6444 | C | VAL | B | 1355 | 19.127 | −12.084 | 6.522 | 1.00 | 65.47 | B |
| 6445 | O | VAL | B | 1355 | 19.471 | −11.525 | 7.578 | 1.00 | 63.12 | B |
| 6446 | N | THR | B | 1356 | 19.812 | −13.132 | 5.927 | 1.00 | 66.86 | B |
| 6447 | CA | THR | B | 1356 | 21.345 | −13.459 | 6.100 | 1.00 | 66.83 | B |
| 6448 | CB | THR | B | 1356 | 21.612 | −15.001 | 6.295 | 1.00 | 67.30 | B |
| 6449 | OG1 | THR | B | 1356 | 23.011 | −15.323 | 5.958 | 1.00 | 64.76 | B |
| 6450 | CG2 | THR | B | 1356 | 20.651 | −15.747 | 5.537 | 1.00 | 60.99 | B |
| 6451 | C | THR | B | 1356 | 22.555 | −12.880 | 5.149 | 1.00 | 68.10 | B |
| 6452 | O | THR | B | 1356 | 22.676 | −11.656 | 4.975 | 1.00 | 68.42 | B |
| 6453 | N | SER | B | 1357 | 23.443 | −13.742 | 4.585 | 1.00 | 67.23 | B |
| 6454 | CA | SER | B | 1357 | 24.636 | −13.291 | 3.855 | 1.00 | 67.37 | B |
| 6455 | CB | SER | B | 1357 | 25.506 | −12.337 | 4.706 | 1.00 | 65.96 | B |
| 6456 | OG | SER | B | 1357 | 26.346 | −12.987 | 5.705 | 1.00 | 67.36 | B |
| 6457 | C | SER | B | 1357 | 25.462 | −14.532 | 3.415 | 1.00 | 68.56 | B |
| 6458 | O | SER | B | 1357 | 26.676 | −14.595 | 3.685 | 1.00 | 71.48 | B |
| 6459 | N | LYS | B | 1358 | 24.852 | −15.527 | 2.789 | 1.00 | 66.55 | B |
| 6460 | CA | LYS | B | 1358 | 25.605 | −16.780 | 2.602 | 1.00 | 65.56 | B |
| 6461 | CB | LYS | B | 1358 | 24.769 | −17.723 | 1.723 | 1.00 | 66.86 | B |
| 6462 | CG | LYS | B | 1358 | 24.552 | −17.312 | 0.195 | 1.00 | 61.22 | B |
| 6463 | CD | LYS | B | 1358 | 23.806 | −16.018 | 0.065 | 1.00 | 59.19 | B |
| 6464 | CE | LYS | B | 1358 | 23.096 | −15.992 | −1.216 | 1.00 | 58.54 | B |
| 6465 | NZ | LYS | B | 1358 | 23.793 | −15.306 | −2.398 | 1.00 | 57.16 | B |
| 6466 | C | LYS | B | 1358 | 27.079 | −16.923 | 2.157 | 1.00 | 66.01 | B |
| 6467 | O | LYS | B | 1358 | 27.907 | −15.994 | 1.906 | 1.00 | 63.35 | B |
| 6468 | N | LYS | B | 1359 | 27.423 | −18.183 | 2.006 | 1.00 | 68.34 | B |
| 6469 | CA | LYS | B | 1359 | 28.795 | −18.411 | 1.558 | 1.00 | 70.85 | B |
| 6470 | CB | LYS | B | 1359 | 29.121 | −19.871 | 1.222 | 1.00 | 71.21 | B |
| 6471 | CG | LYS | B | 1359 | 29.961 | −20.534 | 2.464 | 1.00 | 74.41 | B |
| 6472 | CD | LYS | B | 1359 | 31.573 | −20.399 | 2.294 | 1.00 | 78.71 | B |
| 6473 | CE | LYS | B | 1359 | 32.174 | −18.830 | 2.362 | 1.00 | 82.41 | B |
| 6474 | NZ | LYS | B | 1359 | 31.999 | −17.976 | 3.678 | 1.00 | 74.92 | B |
| 6475 | C | LYS | B | 1359 | 29.204 | −17.436 | 0.509 | 1.00 | 70.72 | B |
| 6476 | O | LYS | B | 1359 | 30.365 | −17.151 | 0.452 | 1.00 | 72.18 | B |
| 6477 | N | ASN | B | 1360 | 28.228 | −16.918 | −0.261 | 1.00 | 72.16 | B |
| 6478 | CA | ASN | B | 1360 | 28.352 | −15.916 | −1.397 | 1.00 | 72.47 | B |
| 6479 | CB | ASN | B | 1360 | 27.004 | −15.928 | −2.198 | 1.00 | 72.06 | B |
| 6480 | CG | ASN | B | 1360 | 26.649 | −14.574 | −2.998 | 1.00 | 71.47 | B |
| 6481 | OD1 | ASN | B | 1360 | 27.314 | −13.539 | −2.925 | 1.00 | 73.12 | B |
| 6482 | ND2 | ASN | B | 1360 | 25.578 | −14.642 | −3.776 | 1.00 | 68.16 | B |
| 6483 | C | ASN | B | 1360 | 28.719 | −14.519 | −0.766 | 1.00 | 72.96 | B |
| 6484 | O | ASN | B | 1360 | 29.748 | −13.810 | −1.156 | 1.00 | 72.10 | B |
| 6485 | N | GLY | B | 1361 | 27.944 | −14.237 | 0.304 | 1.00 | 71.97 | B |
| 6486 | CA | GLY | B | 1361 | 27.980 | −12.986 | 1.049 | 1.00 | 70.10 | B |
| 6487 | C | GLY | B | 1361 | 26.730 | −12.148 | 0.706 | 1.00 | 68.12 | B |
| 6488 | O | GLY | B | 1361 | 26.355 | −11.297 | 1.414 | 1.00 | 67.91 | B |
| 6489 | N | GLN | B | 1362 | 26.122 | −12.318 | −0.458 | 1.00 | 67.50 | B |
| 6490 | CA | GLN | B | 1362 | 24.958 | −11.520 | −0.856 | 1.00 | 64.62 | B |
| 6491 | CB | GLN | B | 1362 | 24.360 | −12.005 | −2.215 | 1.00 | 63.16 | B |
| 6492 | CG | GLN | B | 1362 | 22.941 | −11.407 | −2.547 | 1.00 | 64.07 | B |
| 6493 | CD | GLN | B | 1362 | 21.853 | −12.415 | −2.948 | 1.00 | 65.05 | B |
| 6494 | OE1 | GLN | B | 1362 | 20.686 | −12.056 | −3.210 | 1.00 | 61.54 | B |
| 6495 | NE2 | GLN | B | 1362 | 22.244 | −13.691 | −3.039 | 1.00 | 69.55 | B |
| 6496 | C | GLN | B | 1362 | 24.061 | −11.930 | 0.271 | 1.00 | 63.28 | B |
| 6497 | O | GLN | B | 1362 | 23.514 | −13.042 | 0.261 | 1.00 | 61.85 | B |
| 6498 | N | LEU | B | 1363 | 23.922 | −11.036 | 1.232 | 1.00 | 62.35 | B |
| 6499 | CA | LEU | B | 1363 | 22.855 | −11.148 | 2.233 | 1.00 | 61.69 | B |
| 6500 | CB | LEU | B | 1363 | 22.576 | −9.833 | 2.959 | 1.00 | 59.69 | B |
| 6501 | CG | LEU | B | 1363 | 22.146 | −8.607 | 2.188 | 1.00 | 53.63 | B |
| 6502 | CD1 | LEU | B | 1363 | 20.886 | −7.869 | 2.584 | 1.00 | 48.40 | B |
| 6503 | CD2 | LEU | B | 1363 | 23.342 | −7.745 | 2.503 | 1.00 | 51.96 | B |
| 6504 | C | LEU | B | 1363 | 21.566 | −11.567 | 1.529 | 1.00 | 63.43 | B |
| 6505 | O | LEU | B | 1363 | 21.422 | −11.216 | 0.304 | 1.00 | 64.91 | B |
| 6506 | N | ALA | B | 1364 | 20.663 | −12.313 | 2.218 | 1.00 | 61.95 | B |
| 6507 | CA | ALA | B | 1364 | 19.413 | −12.660 | 1.562 | 1.00 | 62.92 | B |
| 6508 | CB | ALA | B | 1364 | 19.575 | −13.724 | 0.388 | 1.00 | 62.94 | B |
| 6509 | C | ALA | B | 1364 | 18.571 | −13.201 | 2.650 | 1.00 | 64.49 | B |
| 6510 | O | ALA | B | 1364 | 19.070 | −13.400 | 3.803 | 1.00 | 65.76 | B |
| 6511 | N | ALA | B | 1365 | 17.312 | −13.449 | 2.271 | 1.00 | 62.59 | B |
| 6512 | CA | ALA | B | 1365 | 16.309 | −13.895 | 3.130 | 1.00 | 62.06 | B |
| 6513 | CB | ALA | B | 1365 | 15.027 | −13.261 | 2.685 | 1.00 | 63.42 | B |
| 6514 | C | ALA | B | 1365 | 16.098 | −15.355 | 2.944 | 1.00 | 63.05 | B |
| 6515 | O | ALA | B | 1365 | 14.937 | −15.733 | 2.874 | 1.00 | 63.39 | B |
| 6516 | N | SER | B | 1366 | 17.118 | −16.209 | 2.857 | 1.00 | 64.03 | B |
| 6517 | CA | SER | B | 1366 | 16.750 | −17.649 | 2.604 | 1.00 | 66.29 | B |
| 6518 | CB | SER | B | 1366 | 17.796 | −18.585 | 1.915 | 1.00 | 64.24 | B |
| 6519 | OG | SER | B | 1366 | 18.512 | −17.938 | 0.919 | 1.00 | 69.06 | B |
| 6520 | C | SER | B | 1366 | 16.403 | −18.447 | 3.832 | 1.00 | 67.03 | B |
| 6521 | O | SER | B | 1366 | 16.314 | −19.697 | 3.643 | 1.00 | 66.65 | B |
| 6522 | N | VAL | B | 1367 | 16.315 | −17.808 | 5.037 | 1.00 | 67.51 | B |
| 6523 | CA | VAL | B | 1367 | 16.152 | −18.552 | 6.343 | 1.00 | 66.80 | B |
| 6524 | CB | VAL | B | 1367 | 17.476 | −18.501 | 7.225 | 1.00 | 69.16 | B |
| 6525 | CG1 | VAL | B | 1367 | 18.700 | −19.207 | 6.518 | 1.00 | 61.97 | B |
| 6526 | CG2 | VAL | B | 1367 | 17.737 | −17.041 | 7.915 | 1.00 | 67.36 | B |
| 6527 | C | VAL | B | 1367 | 14.904 | −18.205 | 7.199 | 1.00 | 67.03 | B |
| 6528 | O | VAL | B | 1367 | 14.151 | −17.284 | 6.801 | 1.00 | 69.26 | B |
| 6529 | N | GLU | B | 1368 | 14.653 | −18.875 | 8.348 | 1.00 | 65.05 | B |
| 6530 | CA | GLU | B | 1368 | 13.471 | −18.506 | 9.204 | 1.00 | 63.32 | B |
| 6531 | CB | GLU | B | 1368 | 12.216 | −19.241 | 8.834 | 1.00 | 62.42 | B |
| 6532 | CG | GLU | B | 1368 | 11.488 | −18.690 | 7.703 | 1.00 | 63.51 | B |
| 6533 | CD | GLU | B | 1368 | 10.192 | −19.422 | 7.563 | 1.00 | 65.60 | B |
| 6534 | OE1 | GLU | B | 1368 | 10.178 | −20.668 | 7.934 | 1.00 | 67.58 | B |
| 6535 | OE2 | GLU | B | 1368 | 9.193 | −18.778 | 7.150 | 1.00 | 61.79 | B |
| 6536 | C | GLU | B | 1368 | 13.575 | −18.420 | 10.738 | 1.00 | 62.61 | B |
| 6537 | O | GLU | B | 1368 | 12.550 | −18.232 | 11.427 | 1.00 | 62.74 | B |
| 6538 | N | THR | B | 1369 | 14.793 | −18.540 | 11.218 | 1.00 | 62.23 | B |
| 6539 | CA | THR | B | 1369 | 15.180 | −18.549 | 12.625 | 1.00 | 62.47 | B |
| 6540 | CB | THR | B | 1369 | 15.470 | −20.007 | 13.178 | 1.00 | 61.44 | B |
| 6541 | OG1 | THR | B | 1369 | 16.584 | −20.495 | 12.463 | 1.00 | 58.73 | B |
| 6542 | CG2 | THR | B | 1369 | 14.276 | −20.970 | 13.170 | 1.00 | 54.85 | B |
| 6543 | C | THR | B | 1369 | 16.506 | −17.674 | 12.898 | 1.00 | 64.24 | B |
| 6544 | O | THR | B | 1369 | 17.632 | −18.054 | 12.680 | 1.00 | 61.95 | B |
| 6545 | N | ALA | B | 1370 | 16.364 | −16.518 | 13.469 | 1.00 | 68.50 | B |
| 6546 | CA | ALA | B | 1370 | 17.567 | −15.874 | 14.093 | 1.00 | 71.99 | B |
| 6547 | CB | ALA | B | 1370 | 17.127 | −15.044 | 15.448 | 1.00 | 71.75 | B |
| 6548 | C | ALA | B | 1370 | 18.777 | −16.779 | 14.386 | 1.00 | 72.34 | B |
| 6549 | O | ALA | B | 1370 | 19.008 | −17.213 | 15.546 | 1.00 | 74.16 | B |
| 6550 | N | GLY | B | 1371 | 19.587 | −17.043 | 13.385 | 1.00 | 72.53 | B |
| 6551 | CA | GLY | B | 1371 | 20.600 | −18.045 | 13.684 | 1.00 | 73.95 | B |
| 6552 | C | GLY | B | 1371 | 21.620 | −17.393 | 14.628 | 1.00 | 73.61 | B |
| 6553 | O | GLY | B | 1371 | 21.713 | −17.668 | 15.844 | 1.00 | 71.24 | B |
| 6554 | N | ASP | B | 1372 | 22.353 | −16.468 | 14.017 | 1.00 | 73.47 | B |
| 6555 | CA | ASP | B | 1372 | 23.454 | −15.844 | 14.680 | 1.00 | 73.16 | B |
| 6556 | CB | ASP | B | 1372 | 24.270 | −16.963 | 15.459 | 1.00 | 72.79 | B |
| 6557 | CG | ASP | B | 1372 | 25.706 | −16.511 | 15.869 | 1.00 | 71.45 | B |
| 6558 | OD1 | ASP | B | 1372 | 26.581 | −17.381 | 16.072 | 1.00 | 64.97 | B |
| 6559 | OD2 | ASP | B | 1372 | 25.957 | −15.278 | 15.932 | 1.00 | 70.36 | B |
| 6560 | C | ASP | B | 1372 | 24.115 | −15.226 | 13.469 | 1.00 | 72.58 | B |
| 6561 | O | ASP | B | 1372 | 24.934 | −14.324 | 13.514 | 1.00 | 74.52 | B |
| 6562 | N | SER | B | 1373 | 23.752 | −15.809 | 12.371 | 1.00 | 72.73 | B |
| 6563 | CA | SER | B | 1373 | 24.302 | −15.525 | 11.114 | 1.00 | 71.68 | B |
| 6564 | CB | SER | B | 1373 | 24.114 | −16.785 | 10.224 | 1.00 | 72.31 | B |
| 6565 | OG | SER | B | 1373 | 25.079 | −17.814 | 10.356 | 1.00 | 70.43 | B |
| 6566 | C | SER | B | 1373 | 23.277 | −14.543 | 10.572 | 1.00 | 71.30 | B |
| 6567 | O | SER | B | 1373 | 23.581 | −13.885 | 9.555 | 1.00 | 73.55 | B |
| 6568 | N | GLU | B | 1374 | 22.042 | −14.537 | 11.146 | 1.00 | 67.27 | B |
| 6569 | CA | GLU | B | 1374 | 20.859 | −13.925 | 10.473 | 1.00 | 62.03 | B |
| 6570 | CB | GLU | B | 1374 | 19.664 | −14.750 | 10.766 | 1.00 | 62.47 | B |
| 6571 | CG | GLU | B | 1374 | 19.272 | −15.575 | 9.614 | 1.00 | 59.42 | B |
| 6572 | CD | GLU | B | 1374 | 20.410 | −16.385 | 9.071 | 1.00 | 63.76 | B |
| 6573 | OE1 | GLU | B | 1374 | 21.659 | −16.123 | 9.482 | 1.00 | 53.57 | B |
| 6574 | OE2 | GLU | B | 1374 | 19.980 | −17.237 | 8.191 | 1.00 | 52.31 | B |
| 6575 | C | GLU | B | 1374 | 20.576 | −12.539 | 11.017 | 1.00 | 59.87 | B |
| 6576 | O | GLU | B | 1374 | 19.459 | −11.967 | 10.923 | 1.00 | 55.51 | B |
| 6577 | N | LEU | B | 1375 | 21.662 | −12.018 | 11.535 | 1.00 | 57.78 | B |
| 6578 | CA | LEU | B | 1375 | 21.647 | −11.030 | 12.561 | 1.00 | 58.72 | B |
| 6579 | CB | LEU | B | 1375 | 21.954 | −11.705 | 13.917 | 1.00 | 57.99 | B |
| 6580 | CG | LEU | B | 1375 | 21.147 | −12.773 | 14.652 | 1.00 | 47.91 | B |
| 6581 | CD1 | LEU | B | 1375 | 22.080 | −13.415 | 15.427 | 1.00 | 51.60 | B |
| 6582 | CD2 | LEU | B | 1375 | 20.296 | −12.376 | 15.598 | 1.00 | 34.02 | B |
| 6583 | C | LEU | B | 1375 | 22.837 | −10.142 | 12.174 | 1.00 | 59.81 | B |
| 6584 | O | LEU | B | 1375 | 23.932 | −10.677 | 11.804 | 1.00 | 63.60 | B |
| 6585 | N | PHE | B | 1376 | 22.698 | −8.816 | 12.227 | 1.00 | 57.81 | B |
| 6586 | CA | PHE | B | 1376 | 23.753 | −8.052 | 11.637 | 1.00 | 53.63 | B |
| 6587 | CB | PHE | B | 1376 | 23.433 | −7.388 | 10.285 | 1.00 | 52.93 | B |
| 6588 | CG | PHE | B | 1376 | 22.426 | −8.030 | 9.490 | 1.00 | 50.52 | B |
| 6589 | CD1 | PHE | B | 1376 | 21.135 | −8.239 | 9.992 | 1.00 | 50.08 | B |
| 6590 | CD2 | PHE | B | 1376 | 22.748 | −8.411 | 8.170 | 1.00 | 52.56 | B |
| 6591 | CE1 | PHE | B | 1376 | 20.108 | −8.848 | 9.140 | 1.00 | 57.55 | B |
| 6592 | CE2 | PHE | B | 1376 | 21.745 | −8.988 | 7.271 | 1.00 | 56.27 | B |
| 6593 | CZ | PHE | B | 1376 | 20.430 | −9.253 | 7.723 | 1.00 | 51.85 | B |
| 6594 | C | PHE | B | 1376 | 23.844 | −6.981 | 12.593 | 1.00 | 52.46 | B |
| 6595 | O | PHE | B | 1376 | 22.857 | −6.597 | 13.119 | 1.00 | 51.28 | B |
| 6596 | N | LEU | B | 1377 | 25.061 | −6.469 | 12.661 | 1.00 | 52.83 | B |
| 6597 | CA | LEU | B | 1377 | 25.506 | −5.377 | 13.374 | 1.00 | 52.41 | B |
| 6598 | CB | LEU | B | 1377 | 26.964 | −5.614 | 13.576 | 1.00 | 50.98 | B |
| 6599 | CG | LEU | B | 1377 | 27.412 | −4.440 | 14.357 | 1.00 | 49.39 | B |
| 6600 | CD1 | LEU | B | 1377 | 26.529 | −4.440 | 15.537 | 1.00 | 56.86 | B |
| 6601 | CD2 | LEU | B | 1377 | 28.782 | −4.808 | 14.828 | 1.00 | 49.33 | B |
| 6602 | C | LEU | B | 1377 | 25.196 | −4.099 | 12.584 | 1.00 | 55.48 | B |
| 6603 | O | LEU | B | 1377 | 25.046 | −4.043 | 11.268 | 1.00 | 55.53 | B |
| 6604 | N | MET | B | 1378 | 24.972 | −3.072 | 13.424 | 1.00 | 56.40 | B |
| 6605 | CA | MET | B | 1378 | 24.419 | −1.780 | 12.991 | 1.00 | 54.70 | B |
| 6606 | CB | MET | B | 1378 | 23.068 | −1.572 | 13.515 | 1.00 | 52.97 | B |
| 6607 | CG | MET | B | 1378 | 21.943 | −1.462 | 12.394 | 1.00 | 56.09 | B |
| 6608 | SD | MET | B | 1378 | 20.662 | −0.026 | 12.413 | 1.00 | 55.66 | B |
| 6609 | CE | MET | B | 1378 | 21.184 | 0.793 | 14.006 | 1.00 | 37.57 | B |
| 6610 | C | MET | B | 1378 | 25.344 | −0.866 | 13.698 | 1.00 | 55.81 | B |
| 6611 | O | MET | B | 1378 | 25.641 | −1.069 | 14.953 | 1.00 | 53.53 | B |
| 6612 | N | LYS | B | 1379 | 25.857 | 0.118 | 12.898 | 1.00 | 55.79 | B |
| 6613 | CA | LYS | B | 1379 | 26.674 | 1.197 | 13.508 | 1.00 | 54.95 | B |
| 6614 | CB | LYS | B | 1379 | 28.151 | 0.820 | 13.337 | 1.00 | 56.76 | B |
| 6615 | CG | LYS | B | 1379 | 29.327 | 1.862 | 13.635 | 1.00 | 54.51 | B |
| 6616 | CD | LYS | B | 1379 | 30.611 | 1.186 | 13.111 | 1.00 | 52.16 | B |
| 6617 | CE | LYS | B | 1379 | 31.803 | 2.009 | 13.297 | 1.00 | 50.99 | B |
| 6618 | NZ | LYS | B | 1379 | 33.117 | 1.270 | 13.066 | 1.00 | 45.15 | B |
| 6619 | C | LYS | B | 1379 | 26.315 | 2.546 | 12.884 | 1.00 | 55.02 | B |
| 6620 | O | LYS | B | 1379 | 26.473 | 2.716 | 11.698 | 1.00 | 56.78 | B |
| 6621 | N | LEU | B | 1380 | 25.873 | 3.492 | 13.690 | 1.00 | 52.18 | B |
| 6622 | CA | LEU | B | 1380 | 25.411 | 4.801 | 13.215 | 1.00 | 51.40 | B |
| 6623 | CB | LEU | B | 1380 | 24.404 | 5.430 | 14.216 | 1.00 | 49.93 | B |
| 6624 | CG | LEU | B | 1380 | 23.490 | 6.611 | 14.537 | 1.00 | 48.50 | B |
| 6625 | CD1 | LEU | B | 1380 | 23.529 | 7.426 | 15.910 | 1.00 | 43.88 | B |
| 6626 | CD2 | LEU | B | 1380 | 23.226 | 7.538 | 13.505 | 1.00 | 53.16 | B |
| 6627 | C | LEU | B | 1380 | 26.643 | 5.595 | 13.159 | 1.00 | 52.10 | B |
| 6628 | O | LEU | B | 1380 | 27.282 | 5.960 | 14.250 | 1.00 | 55.89 | B |
| 6629 | N | ILE | B | 1381 | 26.976 | 5.950 | 11.915 | 1.00 | 49.70 | B |
| 6630 | CA | ILE | B | 1381 | 28.262 | 6.531 | 11.584 | 1.00 | 46.77 | B |
| 6631 | CB | ILE | B | 1381 | 28.802 | 5.879 | 10.338 | 1.00 | 46.38 | B |
| 6632 | CG2 | ILE | B | 1381 | 29.423 | 4.531 | 10.651 | 1.00 | 38.23 | B |
| 6633 | CG1 | ILE | B | 1381 | 27.694 | 5.815 | 9.215 | 1.00 | 45.60 | B |
| 6634 | CD1 | ILE | B | 1381 | 28.345 | 5.996 | 7.804 | 1.00 | 44.84 | B |
| 6635 | C | ILE | B | 1381 | 28.098 | 8.021 | 11.336 | 1.00 | 49.17 | B |
| 6636 | O | ILE | B | 1381 | 29.042 | 8.831 | 11.103 | 1.00 | 49.72 | B |
| 6637 | N | ASN | B | 1382 | 26.887 | 8.459 | 11.491 | 1.00 | 51.76 | B |
| 6638 | CA | ASN | B | 1382 | 26.668 | 9.901 | 11.415 | 1.00 | 54.39 | B |
| 6639 | CB | ASN | B | 1382 | 25.788 | 10.231 | 10.182 | 1.00 | 53.13 | B |
| 6640 | CG | ASN | B | 1382 | 24.285 | 10.124 | 10.477 | 1.00 | 58.65 | B |
| 6641 | OD1 | ASN | B | 1382 | 23.874 | 9.195 | 11.232 | 1.00 | 60.21 | B |
| 6642 | ND2 | ASN | B | 1382 | 23.422 | 11.137 | 9.919 | 1.00 | 55.37 | B |
| 6643 | C | ASN | B | 1382 | 26.200 | 10.628 | 12.675 | 1.00 | 54.03 | B |
| 6644 | O | ASN | B | 1382 | 25.572 | 11.645 | 12.544 | 1.00 | 57.36 | B |
| 6645 | N | ARG | B | 1383 | 26.469 | 10.132 | 13.873 | 1.00 | 53.79 | B |
| 6646 | CA | ARG | B | 1383 | 26.188 | 10.944 | 15.130 | 1.00 | 52.38 | B |
| 6647 | CB | ARG | B | 1383 | 24.807 | 10.778 | 15.785 | 1.00 | 46.51 | B |
| 6648 | CG | ARG | B | 1383 | 23.800 | 11.424 | 15.055 | 1.00 | 40.39 | B |
| 6649 | CD | ARG | B | 1383 | 23.057 | 12.595 | 15.609 | 1.00 | 39.99 | B |
| 6650 | NE | ARG | B | 1383 | 21.801 | 13.038 | 14.867 | 1.00 | 41.31 | B |
| 6651 | CZ | ARG | B | 1383 | 21.713 | 13.301 | 13.520 | 1.00 | 34.32 | B |
| 6652 | NH1 | ARG | B | 1383 | 22.751 | 13.018 | 12.769 | 1.00 | 31.86 | B |
| 6653 | NH2 | ARG | B | 1383 | 20.625 | 13.769 | 12.907 | 1.00 | 33.69 | B |
| 6654 | C | ARG | B | 1383 | 27.201 | 10.587 | 16.188 | 1.00 | 54.24 | B |
| 6655 | O | ARG | B | 1383 | 26.844 | 10.084 | 17.232 | 1.00 | 55.64 | B |
| 6656 | N | PRO | B | 1384 | 28.458 | 10.899 | 15.934 | 1.00 | 55.27 | B |
| 6657 | CD | PRO | B | 1384 | 29.055 | 11.140 | 14.604 | 1.00 | 56.95 | B |
| 6658 | CA | PRO | B | 1384 | 29.405 | 11.052 | 17.031 | 1.00 | 53.59 | B |
| 6659 | CB | PRO | B | 1384 | 30.700 | 10.787 | 16.359 | 1.00 | 55.05 | B |
| 6660 | CG | PRO | B | 1384 | 30.576 | 11.305 | 14.950 | 1.00 | 57.24 | B |
| 6661 | C | PRO | B | 1384 | 29.325 | 12.412 | 17.844 | 1.00 | 53.25 | B |
| 6662 | O | PRO | B | 1384 | 29.934 | 12.515 | 18.932 | 1.00 | 53.67 | B |
| 6663 | N | ILE | B | 1385 | 28.463 | 13.378 | 17.513 | 1.00 | 52.22 | B |
| 6664 | CA | ILE | B | 1385 | 27.994 | 14.209 | 18.661 | 1.00 | 51.01 | B |
| 6665 | CB | ILE | B | 1385 | 28.336 | 15.645 | 18.578 | 1.00 | 50.87 | B |
| 6666 | CG2 | ILE | B | 1385 | 27.954 | 16.400 | 19.946 | 1.00 | 49.32 | B |
| 6667 | CG1 | ILE | B | 1385 | 29.615 | 15.993 | 17.789 | 1.00 | 49.53 | B |
| 6668 | CD1 | ILE | B | 1385 | 31.001 | 15.491 | 18.234 | 1.00 | 54.91 | B |
| 6669 | C | ILE | B | 1385 | 26.453 | 14.315 | 18.770 | 1.00 | 51.82 | B |
| 6670 | O | ILE | B | 1385 | 25.749 | 14.496 | 17.774 | 1.00 | 54.22 | B |
| 6671 | N | ILE | B | 1386 | 25.918 | 14.312 | 19.974 | 1.00 | 49.03 | B |
| 6672 | CA | ILE | B | 1386 | 24.528 | 13.969 | 20.123 | 1.00 | 48.33 | B |
| 6673 | CB | ILE | B | 1386 | 24.215 | 12.568 | 20.645 | 1.00 | 48.81 | B |
| 6674 | CG2 | ILE | B | 1386 | 22.800 | 12.135 | 20.282 | 1.00 | 51.96 | B |
| 6675 | CG1 | ILE | B | 1386 | 25.114 | 11.597 | 19.996 | 1.00 | 53.54 | B |
| 6676 | CD1 | ILE | B | 1386 | 26.135 | 11.223 | 21.012 | 1.00 | 69.11 | B |
| 6677 | C | ILE | B | 1386 | 23.849 | 14.856 | 21.091 | 1.00 | 45.72 | B |
| 6678 | O | ILE | B | 1386 | 24.412 | 15.325 | 22.042 | 1.00 | 44.28 | B |
| 6679 | N | VAL | B | 1387 | 22.595 | 15.036 | 20.747 | 1.00 | 41.43 | B |
| 6680 | CA | VAL | B | 1387 | 21.865 | 15.956 | 21.343 | 1.00 | 41.38 | B |
| 6681 | CB | VAL | B | 1387 | 21.651 | 17.063 | 20.431 | 1.00 | 41.36 | B |
| 6682 | CG1 | VAL | B | 1387 | 20.364 | 17.834 | 20.935 | 1.00 | 39.46 | B |
| 6683 | CG2 | VAL | B | 1387 | 22.906 | 17.947 | 20.457 | 1.00 | 38.90 | B |
| 6684 | C | VAL | B | 1387 | 20.657 | 15.122 | 21.406 | 1.00 | 42.06 | B |
| 6685 | O | VAL | B | 1387 | 20.402 | 14.502 | 20.428 | 1.00 | 42.08 | B |
| 6686 | N | PHE | B | 1388 | 19.941 | 15.113 | 22.543 | 1.00 | 41.24 | B |
| 6687 | CA | PHE | B | 1388 | 18.874 | 14.323 | 22.694 | 1.00 | 45.93 | B |
| 6688 | CB | PHE | B | 1388 | 19.022 | 13.331 | 23.883 | 1.00 | 47.17 | B |
| 6689 | CG | PHE | B | 1388 | 20.049 | 12.240 | 23.712 | 1.00 | 46.86 | B |
| 6690 | CD1 | PHE | B | 1388 | 19.820 | 11.142 | 22.864 | 1.00 | 41.85 | B |
| 6691 | CD2 | PHE | B | 1388 | 21.234 | 12.345 | 24.295 | 1.00 | 44.67 | B |
| 6692 | CE1 | PHE | B | 1388 | 20.679 | 10.212 | 22.682 | 1.00 | 36.98 | B |
| 6693 | CE2 | PHE | B | 1388 | 22.145 | 11.292 | 24.052 | 1.00 | 53.99 | B |
| 6694 | CZ | PHE | B | 1388 | 21.845 | 10.226 | 23.260 | 1.00 | 42.41 | B |
| 6695 | C | PHE | B | 1388 | 17.782 | 15.306 | 23.091 | 1.00 | 49.86 | B |
| 6696 | O | PHE | B | 1388 | 17.814 | 15.842 | 24.180 | 1.00 | 46.59 | B |
| 6697 | N | ARG | B | 1389 | 16.702 | 15.402 | 22.244 | 1.00 | 55.44 | B |
| 6698 | CA | ARG | B | 1389 | 15.409 | 16.087 | 22.608 | 1.00 | 54.29 | B |
| 6699 | CB | ARG | B | 1389 | 15.099 | 16.920 | 21.433 | 1.00 | 53.25 | B |
| 6700 | CG | ARG | B | 1389 | 14.090 | 17.986 | 21.754 | 1.00 | 55.97 | B |
| 6701 | CD | ARG | B | 1389 | 12.711 | 17.675 | 21.176 | 1.00 | 51.33 | B |
| 6702 | NE | ARG | B | 1389 | 11.892 | 18.843 | 21.323 | 1.00 | 63.58 | B |
| 6703 | CZ | ARG | B | 1389 | 12.026 | 20.016 | 20.659 | 1.00 | 71.68 | B |
| 6704 | NH1 | ARG | B | 1389 | 12.965 | 20.280 | 19.708 | 1.00 | 77.76 | B |
| 6705 | NH2 | ARG | B | 1389 | 11.159 | 20.959 | 20.958 | 1.00 | 72.28 | B |
| 6706 | C | ARG | B | 1389 | 14.199 | 15.200 | 23.067 | 1.00 | 55.51 | B |
| 6707 | O | ARG | B | 1389 | 13.915 | 14.154 | 22.465 | 1.00 | 59.39 | B |
| 6708 | N | GLY | B | 1390 | 13.447 | 15.554 | 24.105 | 1.00 | 54.11 | B |
| 6709 | CA | GLY | B | 1390 | 12.307 | 14.726 | 24.410 | 1.00 | 53.67 | B |
| 6710 | C | GLY | B | 1390 | 11.083 | 15.535 | 24.570 | 1.00 | 56.87 | B |
| 6711 | O | GLY | B | 1390 | 11.193 | 16.806 | 24.428 | 1.00 | 60.20 | B |
| 6712 | N | GLU | B | 1391 | 9.979 | 14.853 | 24.960 | 1.00 | 57.51 | B |
| 6713 | CA | GLU | B | 1391 | 8.613 | 15.416 | 25.126 | 1.00 | 60.57 | B |
| 6714 | CB | GLU | B | 1391 | 7.537 | 14.322 | 25.484 | 1.00 | 60.91 | B |
| 6715 | CG | GLU | B | 1391 | 6.856 | 14.488 | 26.953 | 1.00 | 60.55 | B |
| 6716 | CD | GLU | B | 1391 | 5.485 | 13.727 | 27.199 | 1.00 | 59.22 | B |
| 6717 | OE1 | GLU | B | 1391 | 5.326 | 12.594 | 26.832 | 1.00 | 55.43 | B |
| 6718 | OE2 | GLU | B | 1391 | 4.512 | 14.239 | 27.752 | 1.00 | 59.90 | B |
| 6719 | C | GLU | B | 1391 | 8.340 | 16.459 | 26.146 | 1.00 | 63.69 | B |
| 6720 | O | GLU | B | 1391 | 7.137 | 16.792 | 26.355 | 1.00 | 65.96 | B |
| 6721 | N | HIS | B | 1392 | 9.346 | 16.865 | 26.901 | 1.00 | 65.54 | B |
| 6722 | CA | HIS | B | 1392 | 9.244 | 18.069 | 27.799 | 1.00 | 66.53 | B |
| 6723 | CB | HIS | B | 1392 | 8.807 | 17.705 | 29.295 | 1.00 | 65.26 | B |
| 6724 | CG | HIS | B | 1392 | 7.359 | 17.291 | 29.527 | 1.00 | 61.22 | B |
| 6725 | CD2 | HIS | B | 1392 | 6.826 | 16.083 | 29.787 | 1.00 | 57.18 | B |
| 6726 | ND1 | HIS | B | 1392 | 6.311 | 18.197 | 29.713 | 1.00 | 62.21 | B |
| 6727 | CE1 | HIS | B | 1392 | 5.176 | 17.563 | 29.986 | 1.00 | 53.09 | B |
| 6728 | NE2 | HIS | B | 1392 | 5.465 | 16.269 | 30.007 | 1.00 | 58.41 | B |
| 6729 | C | HIS | B | 1392 | 10.679 | 18.677 | 28.007 | 1.00 | 67.34 | B |
| 6730 | O | HIS | B | 1392 | 10.860 | 19.272 | 29.033 | 1.00 | 66.90 | B |
| 6731 | N | GLY | B | 1393 | 11.728 | 18.436 | 27.185 | 1.00 | 68.35 | B |
| 6732 | CA | GLY | B | 1393 | 13.025 | 19.206 | 27.400 | 1.00 | 69.36 | B |
| 6733 | C | GLY | B | 1393 | 14.154 | 18.586 | 26.597 | 1.00 | 71.18 | B |
| 6734 | O | GLY | B | 1393 | 13.845 | 17.683 | 25.825 | 1.00 | 74.70 | B |
| 6735 | N | PHE | B | 1394 | 15.423 | 19.003 | 26.746 | 1.00 | 68.74 | B |
| 6736 | CA | PHE | B | 1394 | 16.571 | 18.226 | 26.210 | 1.00 | 67.22 | B |
| 6737 | CB | PHE | B | 1394 | 17.526 | 19.148 | 25.452 | 1.00 | 69.16 | B |
| 6738 | CG | PHE | B | 1394 | 16.797 | 19.979 | 24.427 | 1.00 | 72.33 | B |
| 6739 | CD1 | PHE | B | 1394 | 15.691 | 20.843 | 24.864 | 1.00 | 71.36 | B |
| 6740 | CD2 | PHE | B | 1394 | 17.093 | 19.849 | 23.089 | 1.00 | 69.77 | B |
| 6741 | CE1 | PHE | B | 1394 | 14.969 | 21.561 | 24.011 | 1.00 | 68.26 | B |
| 6742 | CE2 | PHE | B | 1394 | 16.306 | 20.541 | 22.192 | 1.00 | 76.72 | B |
| 6743 | CZ | PHE | B | 1394 | 15.260 | 21.425 | 22.637 | 1.00 | 72.29 | B |
| 6744 | C | PHE | B | 1394 | 17.315 | 17.283 | 27.190 | 1.00 | 66.16 | B |
| 6745 | O | PHE | B | 1394 | 16.767 | 16.949 | 28.189 | 1.00 | 65.99 | B |
| 6746 | N | ILE | B | 1395 | 18.504 | 16.762 | 26.902 | 1.00 | 63.93 | B |
| 6747 | CA | ILE | B | 1395 | 19.032 | 15.932 | 27.904 | 1.00 | 64.26 | B |
| 6748 | CB | ILE | B | 1395 | 19.261 | 14.407 | 27.535 | 1.00 | 64.90 | B |
| 6749 | CG2 | ILE | B | 1395 | 19.912 | 13.664 | 28.752 | 1.00 | 62.07 | B |
| 6750 | CG1 | ILE | B | 1395 | 18.038 | 13.642 | 27.213 | 1.00 | 61.51 | B |
| 6751 | CD1 | ILE | B | 1395 | 18.371 | 12.250 | 26.641 | 1.00 | 61.42 | B |
| 6752 | C | ILE | B | 1395 | 20.403 | 16.428 | 28.179 | 1.00 | 66.56 | B |
| 6753 | O | ILE | B | 1395 | 21.266 | 16.210 | 27.335 | 1.00 | 66.32 | B |
| 6754 | N | GLY | B | 1396 | 20.661 | 17.009 | 29.363 | 1.00 | 67.38 | B |
| 6755 | CA | GLY | B | 1396 | 22.056 | 17.541 | 29.619 | 1.00 | 66.81 | B |
| 6756 | C | GLY | B | 1396 | 22.375 | 17.442 | 31.060 | 1.00 | 66.26 | B |
| 6757 | O | GLY | B | 1396 | 21.447 | 17.590 | 31.839 | 1.00 | 68.36 | B |
| 6758 | N | CYS | B | 1397 | 23.616 | 17.116 | 31.447 | 1.00 | 66.10 | B |
| 6759 | CA | CYS | B | 1397 | 24.007 | 17.171 | 32.875 | 1.00 | 64.23 | B |
| 6760 | CB | CYS | B | 1397 | 25.503 | 17.115 | 33.101 | 1.00 | 62.89 | B |
| 6761 | SG | CYS | B | 1397 | 26.606 | 15.982 | 32.159 | 1.00 | 64.20 | B |
| 6762 | C | CYS | B | 1397 | 23.715 | 18.514 | 33.394 | 1.00 | 63.64 | B |
| 6763 | O | CYS | B | 1397 | 24.514 | 19.333 | 33.281 | 1.00 | 65.22 | B |
| 6764 | N | ARG | B | 1398 | 22.593 | 18.767 | 33.981 | 1.00 | 66.40 | B |
| 6765 | CA | ARG | B | 1398 | 22.479 | 19.939 | 34.794 | 1.00 | 69.57 | B |
| 6766 | CB | ARG | B | 1398 | 21.234 | 19.875 | 35.682 | 1.00 | 69.46 | B |
| 6767 | CG | ARG | B | 1398 | 21.013 | 21.210 | 36.438 | 1.00 | 67.76 | B |
| 6768 | CD | ARG | B | 1398 | 21.267 | 20.939 | 37.861 | 1.00 | 63.75 | B |
| 6769 | NE | ARG | B | 1398 | 20.244 | 20.239 | 38.668 | 1.00 | 50.30 | B |
| 6770 | CZ | ARG | B | 1398 | 20.598 | 19.334 | 39.579 | 1.00 | 55.98 | B |
| 6771 | NH1 | ARG | B | 1398 | 21.893 | 18.966 | 39.631 | 1.00 | 58.49 | B |
| 6772 | NH2 | ARG | B | 1398 | 19.706 | 18.708 | 40.379 | 1.00 | 54.57 | B |
| 6773 | C | ARG | B | 1398 | 23.776 | 20.125 | 35.651 | 1.00 | 73.35 | B |
| 6774 | O | ARG | B | 1398 | 24.272 | 19.112 | 36.376 | 1.00 | 70.82 | B |
| 6775 | N | LYS | B | 1399 | 24.335 | 21.379 | 35.533 | 1.00 | 75.68 | B |
| 6776 | CA | LYS | B | 1399 | 25.399 | 21.810 | 36.425 | 1.00 | 77.56 | B |
| 6777 | CB | LYS | B | 1399 | 24.772 | 21.874 | 37.839 | 1.00 | 77.72 | B |
| 6778 | CG | LYS | B | 1399 | 25.198 | 22.955 | 38.822 | 1.00 | 75.53 | B |
| 6779 | CD | LYS | B | 1399 | 24.183 | 24.086 | 38.819 | 1.00 | 75.80 | B |
| 6780 | CE | LYS | B | 1399 | 23.025 | 24.062 | 39.927 | 1.00 | 69.74 | B |
| 6781 | NZ | LYS | B | 1399 | 23.047 | 25.602 | 40.334 | 1.00 | 65.43 | B |
| 6782 | C | LYS | B | 1399 | 26.526 | 20.719 | 36.344 | 1.00 | 79.43 | B |
| 6783 | O | LYS | B | 1399 | 26.889 | 20.184 | 35.182 | 1.00 | 81.18 | B |
| 6784 | N | VAL | B | 1400 | 27.073 | 20.343 | 37.532 | 1.00 | 80.05 | B |
| 6785 | CA | VAL | B | 1400 | 28.323 | 19.440 | 37.665 | 1.00 | 79.11 | B |
| 6786 | CB | VAL | B | 1400 | 29.666 | 20.302 | 37.566 | 1.00 | 79.48 | B |
| 6787 | CG1 | VAL | B | 1400 | 29.696 | 21.293 | 36.312 | 1.00 | 67.15 | B |
| 6788 | CG2 | VAL | B | 1400 | 29.835 | 21.098 | 38.930 | 1.00 | 81.72 | B |
| 6789 | C | VAL | B | 1400 | 28.200 | 18.485 | 38.952 | 1.00 | 80.57 | B |
| 6790 | O | VAL | B | 1400 | 29.152 | 17.838 | 39.539 | 1.00 | 80.14 | B |
| 6791 | N | THR | B | 1401 | 26.928 | 18.405 | 39.316 | 1.00 | 81.55 | B |
| 6792 | CA | THR | B | 1401 | 26.364 | 17.554 | 40.372 | 1.00 | 81.00 | B |
| 6793 | CB | THR | B | 1401 | 24.807 | 18.002 | 40.696 | 1.00 | 82.08 | B |
| 6794 | OG1 | THR | B | 1401 | 24.667 | 19.475 | 40.697 | 1.00 | 74.51 | B |
| 6795 | CG2 | THR | B | 1401 | 24.206 | 17.292 | 42.038 | 1.00 | 81.33 | B |
| 6796 | C | THR | B | 1401 | 26.611 | 16.090 | 39.919 | 1.00 | 80.22 | B |
| 6797 | O | THR | B | 1401 | 26.438 | 15.121 | 40.701 | 1.00 | 80.99 | B |
| 6798 | N | GLY | B | 1402 | 27.170 | 15.985 | 38.705 | 1.00 | 78.16 | B |
| 6799 | CA | GLY | B | 1402 | 27.466 | 14.705 | 38.010 | 1.00 | 74.94 | B |
| 6800 | C | GLY | B | 1402 | 26.155 | 14.343 | 37.317 | 1.00 | 73.55 | B |
| 6801 | O | GLY | B | 1402 | 26.199 | 13.821 | 36.182 | 1.00 | 71.93 | B |
| 6802 | N | THR | B | 1403 | 25.031 | 14.646 | 38.062 | 1.00 | 70.35 | B |
| 6803 | CA | THR | B | 1403 | 23.650 | 14.408 | 37.678 | 1.00 | 67.26 | B |
| 6804 | CB | THR | B | 1403 | 22.542 | 14.836 | 38.842 | 1.00 | 69.11 | B |
| 6805 | OG1 | THR | B | 1403 | 22.069 | 16.175 | 38.707 | 1.00 | 64.57 | B |
| 6806 | CG2 | THR | B | 1403 | 23.023 | 14.526 | 40.356 | 1.00 | 68.24 | B |
| 6807 | C | THR | B | 1403 | 23.310 | 14.775 | 36.163 | 1.00 | 65.20 | B |
| 6808 | O | THR | B | 1403 | 24.128 | 15.336 | 35.521 | 1.00 | 63.01 | B |
| 6809 | N | LEU | B | 1404 | 22.141 | 14.354 | 35.647 | 1.00 | 63.61 | B |
| 6810 | CA | LEU | B | 1404 | 21.773 | 14.243 | 34.198 | 1.00 | 63.06 | B |
| 6811 | CB | LEU | B | 1404 | 22.207 | 12.840 | 33.632 | 1.00 | 62.98 | B |
| 6812 | CG | LEU | B | 1404 | 22.634 | 12.647 | 32.110 | 1.00 | 59.28 | B |
| 6813 | CD1 | LEU | B | 1404 | 23.221 | 13.885 | 31.661 | 1.00 | 54.35 | B |
| 6814 | CD2 | LEU | B | 1404 | 23.589 | 11.585 | 31.832 | 1.00 | 57.47 | B |
| 6815 | C | LEU | B | 1404 | 20.234 | 14.500 | 33.908 | 1.00 | 64.16 | B |
| 6816 | O | LEU | B | 1404 | 19.366 | 13.647 | 34.164 | 1.00 | 64.66 | B |
| 6817 | N | ASP | B | 1405 | 19.848 | 15.670 | 33.395 | 1.00 | 64.15 | B |
| 6818 | CA | ASP | B | 1405 | 18.429 | 15.945 | 33.343 | 1.00 | 63.74 | B |
| 6819 | CB | ASP | B | 1405 | 18.172 | 17.255 | 34.079 | 1.00 | 64.91 | B |
| 6820 | CG | ASP | B | 1405 | 18.211 | 17.087 | 35.612 | 1.00 | 65.49 | B |
| 6821 | OD1 | ASP | B | 1405 | 19.380 | 17.027 | 36.128 | 1.00 | 64.78 | B |
| 6822 | OD2 | ASP | B | 1405 | 17.083 | 17.027 | 36.237 | 1.00 | 62.42 | B |
| 6823 | C | ASP | B | 1405 | 17.794 | 15.878 | 31.929 | 1.00 | 63.68 | B |
| 6824 | O | ASP | B | 1405 | 18.504 | 15.977 | 30.906 | 1.00 | 63.81 | B |
| 6825 | N | ALA | B | 1406 | 16.474 | 15.715 | 31.909 | 1.00 | 63.20 | B |
| 6826 | CA | ALA | B | 1406 | 15.671 | 15.394 | 30.712 | 1.00 | 64.80 | B |
| 6827 | CB | ALA | B | 1406 | 14.614 | 14.333 | 30.988 | 1.00 | 63.22 | B |
| 6828 | C | ALA | B | 1406 | 14.956 | 16.620 | 30.303 | 1.00 | 65.08 | B |
| 6829 | O | ALA | B | 1406 | 14.816 | 16.933 | 29.143 | 1.00 | 66.36 | B |
| 6830 | N | ASN | B | 1407 | 14.435 | 17.330 | 31.256 | 1.00 | 67.23 | B |
| 6831 | CA | ASN | B | 1407 | 13.737 | 18.572 | 30.868 | 1.00 | 66.03 | B |
| 6832 | CB | ASN | B | 1407 | 12.740 | 19.033 | 31.943 | 1.00 | 66.91 | B |
| 6833 | CG | ASN | B | 1407 | 13.282 | 18.944 | 33.291 | 1.00 | 64.18 | B |
| 6834 | OD1 | ASN | B | 1407 | 12.548 | 19.057 | 34.269 | 1.00 | 66.28 | B |
| 6835 | ND2 | ASN | B | 1407 | 14.601 | 18.777 | 33.369 | 1.00 | 61.30 | B |
| 6836 | C | ASN | B | 1407 | 14.535 | 19.761 | 30.497 | 1.00 | 64.47 | B |
| 6837 | O | ASN | B | 1407 | 13.874 | 20.741 | 30.277 | 1.00 | 64.37 | B |
| 6838 | N | ARG | B | 1408 | 15.884 | 19.720 | 30.430 | 1.00 | 62.96 | B |
| 6839 | CA | ARG | B | 1408 | 16.608 | 21.001 | 30.333 | 1.00 | 63.40 | B |
| 6840 | CB | ARG | B | 1408 | 18.121 | 21.001 | 30.044 | 1.00 | 64.00 | B |
| 6841 | CG | ARG | B | 1408 | 18.776 | 19.671 | 30.304 | 1.00 | 68.91 | B |
| 6842 | CD | ARG | B | 1408 | 19.740 | 19.697 | 31.404 | 1.00 | 67.03 | B |
| 6843 | NE | ARG | B | 1408 | 19.602 | 20.814 | 32.274 | 1.00 | 66.75 | B |
| 6844 | CZ | ARG | B | 1408 | 20.649 | 21.423 | 32.847 | 1.00 | 75.82 | B |
| 6845 | NH1 | ARG | B | 1408 | 20.413 | 22.447 | 33.674 | 1.00 | 81.34 | B |
| 6846 | NH2 | ARG | B | 1408 | 21.939 | 21.060 | 32.586 | 1.00 | 74.13 | B |
| 6847 | C | ARG | B | 1408 | 16.014 | 21.487 | 29.132 | 1.00 | 61.84 | B |
| 6848 | O | ARG | B | 1408 | 15.673 | 20.669 | 28.287 | 1.00 | 63.01 | B |
| 6849 | N | SER | B | 1409 | 15.779 | 22.806 | 29.152 | 1.00 | 61.52 | B |
| 6850 | CA | SER | B | 1409 | 15.485 | 23.735 | 28.078 | 1.00 | 60.25 | B |
| 6851 | CB | SER | B | 1409 | 15.139 | 25.075 | 28.719 | 1.00 | 61.22 | B |
| 6852 | OG | SER | B | 1409 | 13.805 | 25.130 | 29.264 | 1.00 | 59.91 | B |
| 6853 | C | SER | B | 1409 | 16.658 | 24.018 | 27.067 | 1.00 | 60.03 | B |
| 6854 | O | SER | B | 1409 | 16.475 | 24.761 | 26.067 | 1.00 | 60.01 | B |
| 6855 | N | SER | B | 1410 | 17.812 | 23.356 | 27.277 | 1.00 | 58.69 | B |
| 6856 | CA | SER | B | 1410 | 18.989 | 23.523 | 26.463 | 1.00 | 55.42 | B |
| 6857 | CB | SER | B | 1410 | 19.645 | 24.779 | 26.990 | 1.00 | 55.33 | B |
| 6858 | OG | SER | B | 1410 | 18.537 | 25.646 | 27.282 | 1.00 | 52.22 | B |
| 6859 | C | SER | B | 1410 | 19.866 | 22.328 | 26.684 | 1.00 | 55.28 | B |
| 6860 | O | SER | B | 1410 | 19.681 | 21.671 | 27.754 | 1.00 | 57.88 | B |
| 6861 | N | TYR | B | 1411 | 20.839 | 22.115 | 25.783 | 1.00 | 51.35 | B |
| 6862 | CA | TYR | B | 1411 | 21.244 | 20.811 | 25.302 | 1.00 | 49.49 | B |
| 6863 | CB | TYR | B | 1411 | 21.288 | 20.671 | 23.744 | 1.00 | 52.91 | B |
| 6864 | CG | TYR | B | 1411 | 20.812 | 21.880 | 22.880 | 1.00 | 56.18 | B |
| 6865 | CD1 | TYR | B | 1411 | 21.553 | 22.321 | 21.819 | 1.00 | 56.32 | B |
| 6866 | CE1 | TYR | B | 1411 | 21.137 | 23.394 | 21.110 | 1.00 | 55.60 | B |
| 6867 | CD2 | TYR | B | 1411 | 19.604 | 22.589 | 23.213 | 1.00 | 63.48 | B |
| 6868 | CE2 | TYR | B | 1411 | 19.188 | 23.659 | 22.515 | 1.00 | 56.75 | B |
| 6869 | CZ | TYR | B | 1411 | 19.935 | 24.049 | 21.455 | 1.00 | 53.66 | B |
| 6870 | OH | TYR | B | 1411 | 19.454 | 25.116 | 20.734 | 1.00 | 44.04 | B |
| 6871 | C | TYR | B | 1411 | 22.605 | 20.813 | 25.555 | 1.00 | 49.11 | B |
| 6872 | O | TYR | B | 1411 | 23.154 | 21.885 | 25.673 | 1.00 | 51.98 | B |
| 6873 | N | ASP | B | 1412 | 23.227 | 19.655 | 25.570 | 1.00 | 46.77 | B |
| 6874 | CA | ASP | B | 1412 | 24.573 | 19.685 | 25.967 | 1.00 | 47.34 | B |
| 6875 | CB | ASP | B | 1412 | 24.803 | 19.108 | 27.376 | 1.00 | 48.19 | B |
| 6876 | CG | ASP | B | 1412 | 24.072 | 19.834 | 28.501 | 1.00 | 53.85 | B |
| 6877 | OD1 | ASP | B | 1412 | 23.848 | 19.106 | 29.556 | 1.00 | 55.84 | B |
| 6878 | OD2 | ASP | B | 1412 | 23.707 | 21.089 | 28.364 | 1.00 | 47.87 | B |
| 6879 | C | ASP | B | 1412 | 25.219 | 18.808 | 24.939 | 1.00 | 47.32 | B |
| 6880 | O | ASP | B | 1412 | 24.908 | 17.696 | 24.628 | 1.00 | 46.52 | B |
| 6881 | N | VAL | B | 1413 | 26.208 | 19.295 | 24.351 | 1.00 | 49.13 | B |
| 6882 | CA | VAL | B | 1413 | 26.679 | 18.427 | 23.356 | 1.00 | 50.81 | B |
| 6883 | CB | VAL | B | 1413 | 27.457 | 19.237 | 22.319 | 1.00 | 47.92 | B |
| 6884 | CG1 | VAL | B | 1413 | 28.647 | 18.478 | 21.913 | 1.00 | 46.74 | B |
| 6885 | CG2 | VAL | B | 1413 | 26.522 | 19.436 | 21.194 | 1.00 | 46.02 | B |
| 6886 | C | VAL | B | 1413 | 27.375 | 17.103 | 24.033 | 1.00 | 53.46 | B |
| 6887 | O | VAL | B | 1413 | 28.525 | 17.135 | 24.638 | 1.00 | 53.97 | B |
| 6888 | N | PHE | B | 1414 | 26.703 | 15.964 | 23.971 | 1.00 | 53.56 | B |
| 6889 | CA | PHE | B | 1414 | 27.363 | 14.840 | 24.591 | 1.00 | 56.16 | B |
| 6890 | CB | PHE | B | 1414 | 26.417 | 13.792 | 25.153 | 1.00 | 55.21 | B |
| 6891 | CG | PHE | B | 1414 | 25.654 | 14.271 | 26.374 | 1.00 | 52.11 | B |
| 6892 | CD1 | PHE | B | 1414 | 26.292 | 14.522 | 27.502 | 1.00 | 49.30 | B |
| 6893 | CD2 | PHE | B | 1414 | 24.266 | 14.425 | 26.363 | 1.00 | 51.29 | B |
| 6894 | CE1 | PHE | B | 1414 | 25.549 | 14.924 | 28.731 | 1.00 | 42.58 | B |
| 6895 | CE2 | PHE | B | 1414 | 23.513 | 14.824 | 27.520 | 1.00 | 46.92 | B |
| 6896 | CZ | PHE | B | 1414 | 24.252 | 15.086 | 28.737 | 1.00 | 46.43 | B |
| 6897 | C | PHE | B | 1414 | 28.058 | 14.438 | 23.394 | 1.00 | 56.97 | B |
| 6898 | O | PHE | B | 1414 | 28.109 | 15.327 | 22.527 | 1.00 | 54.28 | B |
| 6899 | N | GLN | B | 1415 | 28.606 | 13.202 | 23.376 | 1.00 | 58.61 | B |
| 6900 | CA | GLN | B | 1415 | 29.383 | 12.648 | 22.287 | 1.00 | 62.27 | B |
| 6901 | CB | GLN | B | 1415 | 30.786 | 13.286 | 22.311 | 1.00 | 64.29 | B |
| 6902 | CG | GLN | B | 1415 | 32.117 | 12.538 | 22.908 | 1.00 | 64.66 | B |
| 6903 | CD | GLN | B | 1415 | 33.145 | 13.647 | 23.502 | 1.00 | 66.50 | B |
| 6904 | OE1 | GLN | B | 1415 | 32.775 | 14.850 | 23.721 | 1.00 | 72.17 | B |
| 6905 | NE2 | GLN | B | 1415 | 34.383 | 13.232 | 23.791 | 1.00 | 65.67 | B |
| 6906 | C | GLN | B | 1415 | 29.507 | 11.184 | 22.579 | 1.00 | 61.70 | B |
| 6907 | O | GLN | B | 1415 | 29.290 | 10.848 | 23.748 | 1.00 | 63.48 | B |
| 6908 | N | LEU | B | 1416 | 29.941 | 10.336 | 21.617 | 1.00 | 60.36 | B |
| 6909 | CA | LEU | B | 1416 | 29.455 | 8.892 | 21.567 | 1.00 | 58.30 | B |
| 6910 | CB | LEU | B | 1416 | 27.950 | 8.761 | 21.277 | 1.00 | 58.36 | B |
| 6911 | CG | LEU | B | 1416 | 26.965 | 8.180 | 20.214 | 1.00 | 61.09 | B |
| 6912 | CD1 | LEU | B | 1416 | 27.470 | 7.247 | 19.073 | 1.00 | 62.22 | B |
| 6913 | CD2 | LEU | B | 1416 | 25.779 | 7.554 | 20.927 | 1.00 | 57.12 | B |
| 6914 | C | LEU | B | 1416 | 30.123 | 7.923 | 20.710 | 1.00 | 57.89 | B |
| 6915 | O | LEU | B | 1416 | 30.527 | 8.259 | 19.605 | 1.00 | 59.24 | B |
| 6916 | N | GLU | B | 1417 | 30.137 | 6.685 | 21.182 | 1.00 | 58.15 | B |
| 6917 | CA | GLU | B | 1417 | 30.875 | 5.626 | 20.526 | 1.00 | 59.25 | B |
| 6918 | CB | GLU | B | 1417 | 31.773 | 5.004 | 21.514 | 1.00 | 60.64 | B |
| 6919 | CG | GLU | B | 1417 | 33.228 | 5.347 | 21.428 | 1.00 | 58.35 | B |
| 6920 | CD | GLU | B | 1417 | 33.704 | 5.627 | 22.861 | 1.00 | 63.66 | B |
| 6921 | OE1 | GLU | B | 1417 | 33.705 | 4.660 | 23.730 | 1.00 | 60.79 | B |
| 6922 | OE2 | GLU | B | 1417 | 33.960 | 6.869 | 23.164 | 1.00 | 62.00 | B |
| 6923 | C | GLU | B | 1417 | 30.067 | 4.427 | 19.983 | 1.00 | 60.54 | B |
| 6924 | O | GLU | B | 1417 | 28.807 | 4.257 | 20.289 | 1.00 | 61.51 | B |
| 6925 | N | PHE | B | 1418 | 30.768 | 3.558 | 19.226 | 1.00 | 58.81 | B |
| 6926 | CA | PHE | B | 1418 | 30.148 | 2.272 | 18.959 | 1.00 | 59.30 | B |
| 6927 | CB | PHE | B | 1418 | 30.115 | 2.071 | 17.506 | 1.00 | 58.10 | B |
| 6928 | CG | PHE | B | 1418 | 29.469 | 0.897 | 17.153 | 1.00 | 53.26 | B |
| 6929 | CD1 | PHE | B | 1418 | 28.154 | 0.730 | 17.507 | 1.00 | 62.17 | B |
| 6930 | CD2 | PHE | B | 1418 | 30.124 | −0.100 | 16.467 | 1.00 | 51.96 | B |
| 6931 | CE1 | PHE | B | 1418 | 27.387 | −0.580 | 17.061 | 1.00 | 66.76 | B |
| 6932 | CE2 | PHE | B | 1418 | 29.520 | −1.322 | 16.036 | 1.00 | 53.19 | B |
| 6933 | CZ | PHE | B | 1418 | 28.105 | −1.574 | 16.329 | 1.00 | 60.13 | B |
| 6934 | C | PHE | B | 1418 | 30.864 | 1.101 | 19.681 | 1.00 | 61.21 | B |
| 6935 | O | PHE | B | 1418 | 32.101 | 1.150 | 19.819 | 1.00 | 65.68 | B |
| 6936 | N | ASN | B | 1419 | 30.173 | 0.054 | 20.144 | 1.00 | 59.31 | B |
| 6937 | CA | ASN | B | 1419 | 30.859 | −1.030 | 20.947 | 1.00 | 58.27 | B |
| 6938 | CB | ASN | B | 1419 | 30.629 | −0.801 | 22.499 | 1.00 | 59.03 | B |
| 6939 | CG | ASN | B | 1419 | 31.582 | −1.657 | 23.484 | 1.00 | 59.36 | B |
| 6940 | OD1 | ASN | B | 1419 | 32.578 | −1.158 | 24.051 | 1.00 | 53.90 | B |
| 6941 | ND2 | ASN | B | 1419 | 31.151 | −2.848 | 23.794 | 1.00 | 59.42 | B |
| 6942 | C | ASN | B | 1419 | 30.224 | −2.324 | 20.371 | 1.00 | 57.95 | B |
| 6943 | O | ASN | B | 1419 | 29.220 | −2.836 | 20.780 | 1.00 | 59.71 | B |
| 6944 | N | ASP | B | 1420 | 30.785 | −2.823 | 19.327 | 1.00 | 56.88 | B |
| 6945 | CA | ASP | B | 1420 | 30.375 | −4.029 | 18.794 | 1.00 | 55.57 | B |
| 6946 | CB | ASP | B | 1420 | 31.233 | −5.122 | 19.417 | 1.00 | 53.59 | B |
| 6947 | CG | ASP | B | 1420 | 31.522 | −6.234 | 18.408 | 1.00 | 57.38 | B |
| 6948 | OD1 | ASP | B | 1420 | 30.468 | −6.920 | 18.043 | 1.00 | 50.17 | B |
| 6949 | OD2 | ASP | B | 1420 | 32.742 | −6.383 | 17.950 | 1.00 | 50.61 | B |
| 6950 | C | ASP | B | 1420 | 28.790 | −4.268 | 18.535 | 1.00 | 56.78 | B |
| 6951 | O | ASP | B | 1420 | 28.321 | −5.407 | 18.136 | 1.00 | 56.79 | B |
| 6952 | N | GLY | B | 1421 | 27.993 | −3.188 | 18.672 | 1.00 | 55.15 | B |
| 6953 | CA | GLY | B | 1421 | 26.519 | −3.311 | 18.587 | 1.00 | 54.75 | B |
| 6954 | C | GLY | B | 1421 | 25.823 | −2.409 | 19.632 | 1.00 | 55.33 | B |
| 6955 | O | GLY | B | 1421 | 24.559 | −2.364 | 19.702 | 1.00 | 54.17 | B |
| 6956 | N | ALA | B | 1422 | 26.588 | −1.636 | 20.425 | 1.00 | 52.97 | B |
| 6957 | CA | ALA | B | 1422 | 25.988 | −1.004 | 21.584 | 1.00 | 52.58 | B |
| 6958 | CB | ALA | B | 1422 | 26.310 | −1.687 | 22.804 | 1.00 | 48.60 | B |
| 6959 | C | ALA | B | 1422 | 26.589 | 0.349 | 21.600 | 1.00 | 53.85 | B |
| 6960 | O | ALA | B | 1422 | 27.576 | 0.565 | 20.884 | 1.00 | 54.20 | B |
| 6961 | N | TYR | B | 1423 | 26.063 | 1.217 | 22.491 | 1.00 | 53.42 | B |
| 6962 | CA | TYR | B | 1423 | 26.481 | 2.620 | 22.540 | 1.00 | 51.84 | B |
| 6963 | CB | TYR | B | 1423 | 25.265 | 3.515 | 22.004 | 1.00 | 50.76 | B |
| 6964 | CG | TYR | B | 1423 | 25.171 | 3.414 | 20.454 | 1.00 | 46.58 | B |
| 6965 | CD1 | TYR | B | 1423 | 24.311 | 2.505 | 19.885 | 1.00 | 29.54 | B |
| 6966 | CE1 | TYR | B | 1423 | 24.198 | 2.300 | 18.587 | 1.00 | 39.99 | B |
| 6967 | CD2 | TYR | B | 1423 | 26.075 | 4.111 | 19.608 | 1.00 | 39.84 | B |
| 6968 | CE2 | TYR | B | 1423 | 25.982 | 3.891 | 18.195 | 1.00 | 45.77 | B |
| 6969 | CZ | TYR | B | 1423 | 25.042 | 2.926 | 17.687 | 1.00 | 50.96 | B |
| 6970 | OH | TYR | B | 1423 | 24.900 | 2.526 | 16.276 | 1.00 | 54.25 | B |
| 6971 | C | TYR | B | 1423 | 27.015 | 3.127 | 23.908 | 1.00 | 53.05 | B |
| 6972 | O | TYR | B | 1423 | 26.478 | 2.778 | 24.956 | 1.00 | 53.35 | B |
| 6973 | N | ASN | B | 1424 | 27.957 | 4.058 | 23.849 | 1.00 | 52.88 | B |
| 6974 | CA | ASN | B | 1424 | 28.509 | 4.663 | 24.960 | 1.00 | 53.50 | B |
| 6975 | CB | ASN | B | 1424 | 29.962 | 4.162 | 25.018 | 1.00 | 55.13 | B |
| 6976 | CG | ASN | B | 1424 | 30.154 | 2.919 | 25.844 | 1.00 | 55.00 | B |
| 6977 | OD1 | ASN | B | 1424 | 30.467 | 1.864 | 25.303 | 1.00 | 57.51 | B |
| 6978 | ND2 | ASN | B | 1424 | 30.068 | 3.067 | 27.203 | 1.00 | 54.98 | B |
| 6979 | C | ASN | B | 1424 | 28.699 | 6.174 | 24.816 | 1.00 | 53.97 | B |
| 6980 | O | ASN | B | 1424 | 29.525 | 6.528 | 24.033 | 1.00 | 54.86 | B |
| 6981 | N | ILE | B | 1425 | 28.181 | 7.028 | 25.716 | 1.00 | 53.97 | B |
| 6982 | CA | ILE | B | 1425 | 28.116 | 8.472 | 25.659 | 1.00 | 52.60 | B |
| 6983 | CB | ILE | B | 1425 | 26.630 | 8.683 | 25.965 | 1.00 | 51.24 | B |
| 6984 | CG2 | ILE | B | 1425 | 26.237 | 9.993 | 26.577 | 1.00 | 46.60 | B |
| 6985 | CG1 | ILE | B | 1425 | 25.849 | 8.242 | 24.815 | 1.00 | 46.90 | B |
| 6986 | CD1 | ILE | B | 1425 | 24.643 | 7.497 | 25.233 | 1.00 | 52.62 | B |
| 6987 | C | ILE | B | 1425 | 28.866 | 8.921 | 26.910 | 1.00 | 56.81 | B |
| 6988 | O | ILE | B | 1425 | 29.095 | 8.064 | 27.794 | 1.00 | 58.88 | B |
| 6989 | N | LYS | B | 1426 | 29.100 | 10.267 | 27.078 | 1.00 | 59.55 | B |
| 6990 | CA | LYS | B | 1426 | 30.100 | 11.012 | 28.031 | 1.00 | 56.37 | B |
| 6991 | CB | LYS | B | 1426 | 31.574 | 10.581 | 27.967 | 1.00 | 57.42 | B |
| 6992 | CG | LYS | B | 1426 | 32.224 | 10.448 | 26.567 | 1.00 | 58.48 | B |
| 6993 | CD | LYS | B | 1426 | 33.755 | 10.475 | 26.591 | 1.00 | 54.95 | B |
| 6994 | CE | LYS | B | 1426 | 34.151 | 9.082 | 27.067 | 1.00 | 58.49 | B |
| 6995 | NZ | LYS | B | 1426 | 35.524 | 8.561 | 26.782 | 1.00 | 52.36 | B |
| 6996 | C | LYS | B | 1426 | 30.069 | 12.420 | 27.648 | 1.00 | 55.54 | B |
| 6997 | O | LYS | B | 1426 | 29.824 | 12.679 | 26.488 | 1.00 | 56.10 | B |
| 6998 | N | ASP | B | 1427 | 30.227 | 13.303 | 28.660 | 1.00 | 56.66 | B |
| 6999 | CA | ASP | B | 1427 | 29.831 | 14.748 | 28.713 | 1.00 | 54.60 | B |
| 7000 | CB | ASP | B | 1427 | 29.416 | 15.193 | 30.121 | 1.00 | 55.09 | B |
| 7001 | CG | ASP | B | 1427 | 30.202 | 14.485 | 31.298 | 1.00 | 60.79 | B |
| 7002 | OD1 | ASP | B | 1427 | 31.334 | 13.991 | 31.053 | 1.00 | 55.73 | B |
| 7003 | OD2 | ASP | B | 1427 | 29.681 | 14.482 | 32.522 | 1.00 | 66.55 | B |
| 7004 | C | ASP | B | 1427 | 31.062 | 15.367 | 28.265 | 1.00 | 53.44 | B |
| 7005 | O | ASP | B | 1427 | 31.925 | 14.574 | 27.963 | 1.00 | 51.53 | B |
| 7006 | N | SER | B | 1428 | 31.222 | 16.722 | 28.188 | 1.00 | 55.94 | B |
| 7007 | CA | SER | B | 1428 | 32.669 | 17.372 | 28.036 | 1.00 | 56.51 | B |
| 7008 | CB | SER | B | 1428 | 32.675 | 18.914 | 28.194 | 1.00 | 56.61 | B |
| 7009 | OG | SER | B | 1428 | 31.395 | 19.542 | 28.403 | 1.00 | 61.75 | B |
| 7010 | C | SER | B | 1428 | 33.908 | 16.786 | 28.891 | 1.00 | 55.62 | B |
| 7011 | O | SER | B | 1428 | 35.037 | 16.682 | 28.439 | 1.00 | 54.04 | B |
| 7012 | N | THR | B | 1429 | 33.684 | 16.398 | 30.127 | 1.00 | 55.98 | B |
| 7013 | CA | THR | B | 1429 | 34.863 | 16.045 | 30.977 | 1.00 | 56.86 | B |
| 7014 | CB | THR | B | 1429 | 34.624 | 16.193 | 32.506 | 1.00 | 56.94 | B |
| 7015 | OG1 | THR | B | 1429 | 34.254 | 14.878 | 33.021 | 1.00 | 61.29 | B |
| 7016 | CG2 | THR | B | 1429 | 33.515 | 17.338 | 32.888 | 1.00 | 50.41 | B |
| 7017 | C | THR | B | 1429 | 35.341 | 14.599 | 30.743 | 1.00 | 57.82 | B |
| 7018 | O | THR | B | 1429 | 36.540 | 14.300 | 31.015 | 1.00 | 59.41 | B |
| 7019 | N | GLY | B | 1430 | 34.480 | 13.709 | 30.218 | 1.00 | 56.95 | B |
| 7020 | CA | GLY | B | 1430 | 34.960 | 12.285 | 29.988 | 1.00 | 56.60 | B |
| 7021 | C | GLY | B | 1430 | 34.346 | 11.212 | 30.896 | 1.00 | 56.20 | B |
| 7022 | O | GLY | B | 1430 | 34.855 | 10.112 | 30.956 | 1.00 | 54.88 | B |
| 7023 | N | LYS | B | 1431 | 33.256 | 11.530 | 31.597 | 1.00 | 55.31 | B |
| 7024 | CA | LYS | B | 1431 | 32.537 | 10.455 | 32.265 | 1.00 | 59.41 | B |
| 7025 | CB | LYS | B | 1431 | 31.971 | 10.810 | 33.717 | 1.00 | 60.00 | B |
| 7026 | CG | LYS | B | 1431 | 32.601 | 11.990 | 34.576 | 1.00 | 63.56 | B |
| 7027 | CD | LYS | B | 1431 | 31.610 | 13.396 | 34.614 | 1.00 | 66.10 | B |
| 7028 | CE | LYS | B | 1431 | 31.985 | 14.550 | 35.704 | 1.00 | 62.32 | B |
| 7029 | NZ | LYS | B | 1431 | 33.515 | 14.769 | 35.988 | 1.00 | 61.01 | B |
| 7030 | C | LYS | B | 1431 | 31.427 | 9.687 | 31.341 | 1.00 | 59.25 | B |
| 7031 | O | LYS | B | 1431 | 30.752 | 10.293 | 30.514 | 1.00 | 57.95 | B |
| 7032 | N | TYR | B | 1432 | 31.264 | 8.367 | 31.571 | 1.00 | 57.70 | B |
| 7033 | CA | TYR | B | 1432 | 30.506 | 7.457 | 30.735 | 1.00 | 56.10 | B |
| 7034 | CB | TYR | B | 1432 | 31.137 | 6.068 | 30.756 | 1.00 | 54.71 | B |
| 7035 | CG | TYR | B | 1432 | 31.951 | 5.930 | 29.487 | 1.00 | 56.73 | B |
| 7036 | CD1 | TYR | B | 1432 | 31.319 | 6.159 | 28.188 | 1.00 | 51.09 | B |
| 7037 | CE1 | TYR | B | 1432 | 32.122 | 6.073 | 26.947 | 1.00 | 56.13 | B |
| 7038 | CD2 | TYR | B | 1432 | 33.334 | 5.625 | 29.529 | 1.00 | 51.99 | B |
| 7039 | CE2 | TYR | B | 1432 | 34.122 | 5.599 | 28.268 | 1.00 | 58.49 | B |
| 7040 | CZ | TYR | B | 1432 | 33.519 | 5.837 | 26.997 | 1.00 | 56.05 | B |
| 7041 | OH | TYR | B | 1432 | 34.237 | 5.746 | 25.821 | 1.00 | 54.45 | B |
| 7042 | C | TYR | B | 1432 | 29.175 | 7.425 | 31.314 | 1.00 | 55.69 | B |
| 7043 | O | TYR | B | 1432 | 29.078 | 7.522 | 32.582 | 1.00 | 59.09 | B |
| 7044 | N | TRP | B | 1433 | 28.116 | 7.448 | 30.479 | 1.00 | 53.45 | B |
| 7045 | CA | TRP | B | 1433 | 26.751 | 7.414 | 31.052 | 1.00 | 51.73 | B |
| 7046 | CB | TRP | B | 1433 | 25.679 | 7.283 | 29.965 | 1.00 | 49.10 | B |
| 7047 | CG | TRP | B | 1433 | 25.346 | 8.639 | 29.381 | 1.00 | 53.31 | B |
| 7048 | CD2 | TRP | B | 1433 | 24.154 | 9.000 | 28.708 | 1.00 | 43.23 | B |
| 7049 | CE2 | TRP | B | 1433 | 24.245 | 10.373 | 28.411 | 1.00 | 47.22 | B |
| 7050 | CE3 | TRP | B | 1433 | 22.983 | 8.349 | 28.477 | 1.00 | 38.46 | B |
| 7051 | CD1 | TRP | B | 1433 | 26.200 | 9.788 | 29.279 | 1.00 | 52.73 | B |
| 7052 | NE1 | TRP | B | 1433 | 25.511 | 10.809 | 28.719 | 1.00 | 43.22 | B |
| 7053 | CZ2 | TRP | B | 1433 | 23.139 | 11.104 | 27.880 | 1.00 | 45.57 | B |
| 7054 | CZ3 | TRP | B | 1433 | 21.977 | 8.999 | 27.991 | 1.00 | 44.46 | B |
| 7055 | CH2 | TRP | B | 1433 | 22.026 | 10.418 | 27.705 | 1.00 | 45.14 | B |
| 7056 | C | TRP | B | 1433 | 26.905 | 6.168 | 31.980 | 1.00 | 51.87 | B |
| 7057 | O | TRP | B | 1433 | 27.451 | 5.078 | 31.517 | 1.00 | 50.68 | B |
| 7058 | N | THR | B | 1434 | 26.535 | 6.362 | 33.250 | 1.00 | 51.13 | B |
| 7059 | CA | THR | B | 1434 | 26.401 | 5.262 | 34.206 | 1.00 | 51.35 | B |
| 7060 | CB | THR | B | 1434 | 27.175 | 5.403 | 35.647 | 1.00 | 52.30 | B |
| 7061 | OG1 | THR | B | 1434 | 27.354 | 6.778 | 36.159 | 1.00 | 41.93 | B |
| 7062 | CG2 | THR | B | 1434 | 28.197 | 4.353 | 35.876 | 1.00 | 48.37 | B |
| 7063 | C | THR | B | 1434 | 25.113 | 5.411 | 34.935 | 1.00 | 54.04 | B |
| 7064 | O | THR | B | 1434 | 24.817 | 6.609 | 35.425 | 1.00 | 52.57 | B |
| 7065 | N | VAL | B | 1435 | 24.523 | 4.207 | 35.226 | 1.00 | 52.29 | B |
| 7066 | CA | VAL | B | 1435 | 23.392 | 4.151 | 36.110 | 1.00 | 51.88 | B |
| 7067 | CB | VAL | B | 1435 | 22.407 | 3.264 | 35.486 | 1.00 | 51.40 | B |
| 7068 | CG1 | VAL | B | 1435 | 22.902 | 2.981 | 34.149 | 1.00 | 44.26 | B |
| 7069 | CG2 | VAL | B | 1435 | 22.226 | 1.984 | 36.316 | 1.00 | 51.63 | B |
| 7070 | C | VAL | B | 1435 | 23.564 | 3.821 | 37.620 | 1.00 | 54.42 | B |
| 7071 | O | VAL | B | 1435 | 24.469 | 3.088 | 37.990 | 1.00 | 55.82 | B |
| 7072 | N | GLY | B | 1436 | 22.621 | 4.294 | 38.484 | 1.00 | 55.72 | B |
| 7073 | CA | GLY | B | 1436 | 22.620 | 4.068 | 39.945 | 1.00 | 53.69 | B |
| 7074 | C | GLY | B | 1436 | 21.741 | 2.930 | 40.548 | 1.00 | 54.20 | B |
| 7075 | O | GLY | B | 1436 | 21.355 | 1.970 | 39.850 | 1.00 | 50.59 | B |
| 7076 | N | SER | B | 1437 | 21.442 | 3.066 | 41.884 | 1.00 | 54.80 | B |
| 7077 | CA | SER | B | 1437 | 20.843 | 2.030 | 42.737 | 1.00 | 53.74 | B |
| 7078 | CB | SER | B | 1437 | 21.416 | 2.120 | 44.185 | 1.00 | 54.61 | B |
| 7079 | OG | SER | B | 1437 | 20.465 | 1.895 | 45.258 | 1.00 | 48.21 | B |
| 7080 | C | SER | B | 1437 | 19.331 | 2.197 | 42.614 | 1.00 | 55.58 | B |
| 7081 | O | SER | B | 1437 | 18.586 | 1.240 | 42.945 | 1.00 | 56.99 | B |
| 7082 | N | ASP | B | 1438 | 18.895 | 3.392 | 42.148 | 1.00 | 55.66 | B |
| 7083 | CA | ASP | B | 1438 | 17.544 | 3.671 | 41.508 | 1.00 | 56.46 | B |
| 7084 | CB | ASP | B | 1438 | 17.039 | 4.972 | 42.106 | 1.00 | 56.26 | B |
| 7085 | CG | ASP | B | 1438 | 18.080 | 6.069 | 41.974 | 1.00 | 63.81 | B |
| 7086 | OD1 | ASP | B | 1438 | 18.316 | 6.650 | 40.863 | 1.00 | 63.50 | B |
| 7087 | OD2 | ASP | B | 1438 | 18.730 | 6.295 | 43.035 | 1.00 | 78.35 | B |
| 7088 | C | ASP | B | 1438 | 17.666 | 4.031 | 40.020 | 1.00 | 54.34 | B |
| 7089 | O | ASP | B | 1438 | 17.505 | 5.220 | 39.670 | 1.00 | 55.87 | B |
| 7090 | N | SER | B | 1439 | 18.060 | 3.115 | 39.177 | 1.00 | 51.07 | B |
| 7091 | CA | SER | B | 1439 | 18.138 | 3.382 | 37.752 | 1.00 | 51.63 | B |
| 7092 | CB | SER | B | 1439 | 16.871 | 2.838 | 37.088 | 1.00 | 51.33 | B |
| 7093 | OG | SER | B | 1439 | 16.234 | 2.007 | 38.049 | 1.00 | 56.65 | B |
| 7094 | C | SER | B | 1439 | 18.400 | 4.905 | 37.265 | 1.00 | 51.97 | B |
| 7095 | O | SER | B | 1439 | 18.406 | 5.203 | 35.993 | 1.00 | 49.08 | B |
| 7096 | N | ALA | B | 1440 | 18.635 | 5.805 | 38.259 | 1.00 | 50.17 | B |
| 7097 | CA | ALA | B | 1440 | 19.263 | 7.123 | 37.925 | 1.00 | 51.83 | B |
| 7098 | CB | ALA | B | 1440 | 19.880 | 7.940 | 39.256 | 1.00 | 48.64 | B |
| 7099 | C | ALA | B | 1440 | 20.301 | 6.966 | 36.755 | 1.00 | 51.32 | B |
| 7100 | O | ALA | B | 1440 | 20.828 | 5.855 | 36.593 | 1.00 | 52.84 | B |
| 7101 | N | VAL | B | 1441 | 20.529 | 7.966 | 35.900 | 1.00 | 49.82 | B |
| 7102 | CA | VAL | B | 1441 | 21.621 | 7.793 | 34.910 | 1.00 | 47.95 | B |
| 7103 | CB | VAL | B | 1441 | 21.256 | 7.717 | 33.296 | 1.00 | 48.83 | B |
| 7104 | CG1 | VAL | B | 1441 | 22.484 | 7.663 | 32.420 | 1.00 | 45.67 | B |
| 7105 | CG2 | VAL | B | 1441 | 20.368 | 6.562 | 32.774 | 1.00 | 43.37 | B |
| 7106 | C | VAL | B | 1441 | 22.396 | 9.073 | 35.101 | 1.00 | 47.96 | B |
| 7107 | O | VAL | B | 1441 | 21.807 | 10.136 | 35.301 | 1.00 | 45.86 | B |
| 7108 | N | THR | B | 1442 | 23.738 | 8.950 | 35.003 | 1.00 | 47.45 | B |
| 7109 | CA | THR | B | 1442 | 24.623 | 10.003 | 35.151 | 1.00 | 45.49 | B |
| 7110 | CB | THR | B | 1442 | 24.921 | 10.251 | 36.705 | 1.00 | 43.52 | B |
| 7111 | OG1 | THR | B | 1442 | 25.305 | 9.036 | 37.250 | 1.00 | 47.75 | B |
| 7112 | CG2 | THR | B | 1442 | 23.673 | 10.585 | 37.488 | 1.00 | 38.83 | B |
| 7113 | C | THR | B | 1442 | 25.851 | 9.648 | 34.258 | 1.00 | 48.81 | B |
| 7114 | O | THR | B | 1442 | 26.187 | 8.431 | 33.936 | 1.00 | 44.75 | B |
| 7115 | N | SER | B | 1443 | 26.537 | 10.718 | 33.860 | 1.00 | 52.00 | B |
| 7116 | CA | SER | B | 1443 | 27.572 | 10.500 | 32.897 | 1.00 | 58.20 | B |
| 7117 | CB | SER | B | 1443 | 27.615 | 11.711 | 31.894 | 1.00 | 59.21 | B |
| 7118 | OG | SER | B | 1443 | 28.299 | 11.352 | 30.685 | 1.00 | 63.08 | B |
| 7119 | C | SER | B | 1443 | 28.835 | 10.475 | 33.722 | 1.00 | 60.92 | B |
| 7120 | O | SER | B | 1443 | 29.943 | 10.207 | 33.225 | 1.00 | 59.33 | B |
| 7121 | N | SER | B | 1444 | 28.624 | 10.948 | 34.959 | 1.00 | 64.82 | B |
| 7122 | CA | SER | B | 1444 | 29.494 | 10.799 | 36.121 | 1.00 | 67.47 | B |
| 7123 | CB | SER | B | 1444 | 28.657 | 11.196 | 37.397 | 1.00 | 69.94 | B |
| 7124 | OG | SER | B | 1444 | 28.338 | 12.649 | 37.469 | 1.00 | 72.73 | B |
| 7125 | C | SER | B | 1444 | 29.887 | 9.319 | 36.066 | 1.00 | 68.45 | B |
| 7126 | O | SER | B | 1444 | 29.134 | 8.472 | 35.489 | 1.00 | 69.57 | B |
| 7127 | N | GLY | B | 1445 | 31.111 | 9.002 | 36.506 | 1.00 | 69.09 | B |
| 7128 | CA | GLY | B | 1445 | 31.600 | 7.608 | 36.481 | 1.00 | 67.75 | B |
| 7129 | C | GLY | B | 1445 | 32.336 | 7.276 | 35.248 | 1.00 | 67.55 | B |
| 7130 | O | GLY | B | 1445 | 31.914 | 6.539 | 34.403 | 1.00 | 67.31 | B |
| 7131 | N | ASP | B | 1446 | 33.505 | 7.852 | 35.173 | 1.00 | 68.15 | B |
| 7132 | CA | ASP | B | 1446 | 34.514 | 7.465 | 34.183 | 1.00 | 67.39 | B |
| 7133 | CB | ASP | B | 1446 | 35.833 | 8.286 | 34.493 | 1.00 | 66.62 | B |
| 7134 | CG | ASP | B | 1446 | 35.628 | 9.907 | 34.500 | 1.00 | 61.40 | B |
| 7135 | OD1 | ASP | B | 1446 | 36.676 | 10.616 | 34.390 | 1.00 | 48.79 | B |
| 7136 | OD2 | ASP | B | 1446 | 34.451 | 10.421 | 34.553 | 1.00 | 49.99 | B |
| 7137 | C | ASP | B | 1446 | 34.761 | 5.901 | 34.108 | 1.00 | 68.59 | B |
| 7138 | O | ASP | B | 1446 | 35.757 | 5.377 | 34.767 | 1.00 | 67.10 | B |
| 7139 | N | THR | B | 1447 | 33.878 | 5.209 | 33.310 | 1.00 | 68.95 | B |
| 7140 | CA | THR | B | 1447 | 33.720 | 3.661 | 33.194 | 1.00 | 69.23 | B |
| 7141 | CB | THR | B | 1447 | 33.329 | 3.031 | 34.639 | 1.00 | 70.85 | B |
| 7142 | OG1 | THR | B | 1447 | 34.142 | 3.611 | 35.712 | 1.00 | 70.45 | B |
| 7143 | CG2 | THR | B | 1447 | 33.319 | 1.457 | 34.662 | 1.00 | 69.40 | B |
| 7144 | C | THR | B | 1447 | 32.728 | 3.049 | 32.023 | 1.00 | 69.56 | B |
| 7145 | O | THR | B | 1447 | 31.474 | 3.289 | 31.990 | 1.00 | 67.02 | B |
| 7146 | N | PRO | B | 1448 | 33.298 | 2.330 | 31.015 | 1.00 | 68.73 | B |
| 7147 | CD | PRO | B | 1448 | 34.723 | 1.950 | 30.855 | 1.00 | 69.37 | B |
| 7148 | CA | PRO | B | 1448 | 32.495 | 1.969 | 29.815 | 1.00 | 67.70 | B |
| 7149 | CB | PRO | B | 1448 | 33.535 | 1.275 | 28.872 | 1.00 | 68.21 | B |
| 7150 | CG | PRO | B | 1448 | 34.915 | 1.779 | 29.335 | 1.00 | 66.71 | B |
| 7151 | C | PRO | B | 1448 | 31.325 | 1.007 | 30.065 | 1.00 | 66.91 | B |
| 7152 | O | PRO | B | 1448 | 31.553 | −0.276 | 30.241 | 1.00 | 65.10 | B |
| 7153 | N | VAL | B | 1449 | 30.086 | 1.591 | 30.055 | 1.00 | 64.63 | B |
| 7154 | CA | VAL | B | 1449 | 28.808 | 0.778 | 29.909 | 1.00 | 60.31 | B |
| 7155 | CB | VAL | B | 1449 | 27.826 | 1.071 | 31.064 | 1.00 | 60.49 | B |
| 7156 | CG1 | VAL | B | 1449 | 28.162 | 0.265 | 32.178 | 1.00 | 59.14 | B |
| 7157 | CG2 | VAL | B | 1449 | 27.758 | 2.572 | 31.413 | 1.00 | 56.18 | B |
| 7158 | C | VAL | B | 1449 | 28.107 | 0.829 | 28.479 | 1.00 | 60.46 | B |
| 7159 | O | VAL | B | 1449 | 28.409 | 1.685 | 27.717 | 1.00 | 61.94 | B |
| 7160 | N | ASP | B | 1450 | 27.166 | −0.056 | 28.143 | 1.00 | 59.32 | B |
| 7161 | CA | ASP | B | 1450 | 26.606 | −0.218 | 26.764 | 1.00 | 56.26 | B |
| 7162 | CB | ASP | B | 1450 | 26.756 | −1.679 | 26.203 | 1.00 | 54.81 | B |
| 7163 | CG | ASP | B | 1450 | 28.135 | −1.928 | 25.685 | 1.00 | 60.05 | B |
| 7164 | OD1 | ASP | B | 1450 | 28.580 | −3.058 | 25.283 | 1.00 | 58.35 | B |
| 7165 | OD2 | ASP | B | 1450 | 28.815 | −0.881 | 25.729 | 1.00 | 66.19 | B |
| 7166 | C | ASP | B | 1450 | 25.159 | 0.140 | 26.723 | 1.00 | 53.17 | B |
| 7167 | O | ASP | B | 1450 | 24.226 | −0.658 | 27.061 | 1.00 | 51.38 | B |
| 7168 | N | PHE | B | 1451 | 24.870 | 1.268 | 26.133 | 1.00 | 51.88 | B |
| 7169 | CA | PHE | B | 1451 | 23.462 | 1.416 | 25.928 | 1.00 | 49.79 | B |
| 7170 | CB | PHE | B | 1451 | 23.057 | 2.799 | 26.015 | 1.00 | 49.88 | B |
| 7171 | CG | PHE | B | 1451 | 23.443 | 3.492 | 27.336 | 1.00 | 48.88 | B |
| 7172 | CD1 | PHE | B | 1451 | 24.755 | 3.494 | 27.806 | 1.00 | 48.02 | B |
| 7173 | CD2 | PHE | B | 1451 | 22.532 | 4.222 | 28.013 | 1.00 | 49.64 | B |
| 7174 | CE1 | PHE | B | 1451 | 25.098 | 4.179 | 28.866 | 1.00 | 47.05 | B |
| 7175 | CE2 | PHE | B | 1451 | 22.897 | 4.875 | 29.176 | 1.00 | 52.47 | B |
| 7176 | CZ | PHE | B | 1451 | 24.174 | 4.899 | 29.572 | 1.00 | 47.93 | B |
| 7177 | C | PHE | B | 1451 | 23.129 | 0.853 | 24.629 | 1.00 | 51.05 | B |
| 7178 | O | PHE | B | 1451 | 24.070 | 0.520 | 23.780 | 1.00 | 49.64 | B |
| 7179 | N | PHE | B | 1452 | 21.807 | 0.645 | 24.491 | 1.00 | 53.19 | B |
| 7180 | CA | PHE | B | 1452 | 21.190 | 0.059 | 23.244 | 1.00 | 56.62 | B |
| 7181 | CB | PHE | B | 1452 | 20.516 | −1.294 | 23.538 | 1.00 | 57.59 | B |
| 7182 | CG | PHE | B | 1452 | 21.520 | −2.280 | 23.978 | 1.00 | 59.48 | B |
| 7183 | CD1 | PHE | B | 1452 | 22.300 | −2.021 | 25.192 | 1.00 | 60.99 | B |
| 7184 | CD2 | PHE | B | 1452 | 21.913 | −3.263 | 23.107 | 1.00 | 58.76 | B |
| 7185 | CE1 | PHE | B | 1452 | 23.359 | −2.844 | 25.604 | 1.00 | 57.95 | B |
| 7186 | CE2 | PHE | B | 1452 | 22.923 | −4.097 | 23.490 | 1.00 | 65.31 | B |
| 7187 | CZ | PHE | B | 1452 | 23.666 | −3.892 | 24.796 | 1.00 | 62.58 | B |
| 7188 | C | PHE | B | 1452 | 20.259 | 0.997 | 22.702 | 1.00 | 57.06 | B |
| 7189 | O | PHE | B | 1452 | 19.419 | 1.415 | 23.471 | 1.00 | 63.15 | B |
| 7190 | N | PHE | B | 1453 | 20.426 | 1.436 | 21.471 | 1.00 | 54.98 | B |
| 7191 | CA | PHE | B | 1453 | 19.463 | 2.351 | 20.845 | 1.00 | 52.18 | B |
| 7192 | CB | PHE | B | 1453 | 20.182 | 3.268 | 19.846 | 1.00 | 54.43 | B |
| 7193 | CG | PHE | B | 1453 | 20.811 | 4.410 | 20.453 | 1.00 | 53.97 | B |
| 7194 | CD1 | PHE | B | 1453 | 20.687 | 4.633 | 21.787 | 1.00 | 54.57 | B |
| 7195 | CD2 | PHE | B | 1453 | 21.625 | 5.188 | 19.706 | 1.00 | 51.78 | B |
| 7196 | CE1 | PHE | B | 1453 | 21.391 | 5.733 | 22.351 | 1.00 | 58.98 | B |
| 7197 | CE2 | PHE | B | 1453 | 22.307 | 6.234 | 20.233 | 1.00 | 51.46 | B |
| 7198 | CZ | PHE | B | 1453 | 22.226 | 6.536 | 21.532 | 1.00 | 52.10 | B |
| 7199 | C | PHE | B | 1453 | 18.518 | 1.630 | 19.909 | 1.00 | 50.52 | B |
| 7200 | O | PHE | B | 1453 | 18.940 | 0.821 | 19.155 | 1.00 | 49.02 | B |
| 7201 | N | GLU | B | 1454 | 17.268 | 2.049 | 19.841 | 1.00 | 51.26 | B |
| 7202 | CA | GLU | B | 1454 | 16.192 | 1.387 | 19.036 | 1.00 | 50.28 | B |
| 7203 | CB | GLU | B | 1454 | 15.150 | 0.791 | 19.970 | 1.00 | 49.69 | B |
| 7204 | CG | GLU | B | 1454 | 15.863 | −0.129 | 21.040 | 1.00 | 50.67 | B |
| 7205 | CD | GLU | B | 1454 | 15.058 | −1.251 | 21.610 | 1.00 | 55.45 | B |
| 7206 | OE1 | GLU | B | 1454 | 15.791 | −2.145 | 22.157 | 1.00 | 53.20 | B |
| 7207 | OE2 | GLU | B | 1454 | 13.766 | −1.292 | 21.443 | 1.00 | 53.30 | B |
| 7208 | C | GLU | B | 1454 | 15.650 | 2.532 | 18.314 | 1.00 | 50.70 | B |
| 7209 | O | GLU | B | 1454 | 15.437 | 3.604 | 18.988 | 1.00 | 51.25 | B |
| 7210 | N | PHE | B | 1455 | 15.614 | 2.448 | 16.950 | 1.00 | 50.53 | B |
| 7211 | CA | PHE | B | 1455 | 15.011 | 3.554 | 16.155 | 1.00 | 48.49 | B |
| 7212 | CB | PHE | B | 1455 | 15.750 | 3.934 | 15.010 | 1.00 | 45.46 | B |
| 7213 | CG | PHE | B | 1455 | 17.170 | 4.315 | 15.233 | 1.00 | 44.30 | B |
| 7214 | CD1 | PHE | B | 1455 | 18.221 | 3.300 | 15.221 | 1.00 | 48.56 | B |
| 7215 | CD2 | PHE | B | 1455 | 17.531 | 5.671 | 15.305 | 1.00 | 42.67 | B |
| 7216 | CE1 | PHE | B | 1455 | 19.567 | 3.623 | 15.343 | 1.00 | 43.12 | B |
| 7217 | CE2 | PHE | B | 1455 | 18.878 | 6.100 | 15.464 | 1.00 | 36.47 | B |
| 7218 | CZ | PHE | B | 1455 | 19.916 | 5.067 | 15.484 | 1.00 | 45.39 | B |
| 7219 | C | PHE | B | 1455 | 13.558 | 3.239 | 15.753 | 1.00 | 52.06 | B |
| 7220 | O | PHE | B | 1455 | 13.216 | 2.601 | 14.741 | 1.00 | 54.58 | B |
| 7221 | N | CYS | B | 1456 | 12.714 | 3.748 | 16.610 | 1.00 | 53.29 | B |
| 7222 | CA | CYS | B | 1456 | 11.388 | 3.458 | 16.802 | 1.00 | 54.89 | B |
| 7223 | CB | CYS | B | 1456 | 11.281 | 3.676 | 18.348 | 1.00 | 55.17 | B |
| 7224 | SG | CYS | B | 1456 | 11.848 | 2.277 | 18.972 | 1.00 | 62.12 | B |
| 7225 | C | CYS | B | 1456 | 10.543 | 4.481 | 16.132 | 1.00 | 55.04 | B |
| 7226 | O | CYS | B | 1456 | 9.291 | 4.373 | 16.109 | 1.00 | 55.69 | B |
| 7227 | N | ASP | B | 1457 | 11.156 | 5.558 | 15.723 | 1.00 | 55.08 | B |
| 7228 | CA | ASP | B | 1457 | 10.363 | 6.476 | 15.059 | 1.00 | 58.38 | B |
| 7229 | CB | ASP | B | 1457 | 10.037 | 7.603 | 15.964 | 1.00 | 60.77 | B |
| 7230 | CG | ASP | B | 1457 | 9.128 | 7.225 | 17.032 | 1.00 | 66.77 | B |
| 7231 | OD1 | ASP | B | 1457 | 7.900 | 7.675 | 16.832 | 1.00 | 71.86 | B |
| 7232 | OD2 | ASP | B | 1457 | 9.641 | 6.465 | 17.990 | 1.00 | 61.07 | B |
| 7233 | C | ASP | B | 1457 | 11.298 | 7.090 | 14.138 | 1.00 | 60.45 | B |
| 7234 | O | ASP | B | 1457 | 12.555 | 6.904 | 14.270 | 1.00 | 59.46 | B |
| 7235 | N | TYR | B | 1458 | 10.675 | 7.966 | 13.307 | 1.00 | 61.25 | B |
| 7236 | CA | TYR | B | 1458 | 11.306 | 8.412 | 12.065 | 1.00 | 61.52 | B |
| 7237 | CB | TYR | B | 1458 | 10.347 | 9.024 | 11.052 | 1.00 | 61.41 | B |
| 7238 | CG | TYR | B | 1458 | 9.634 | 10.136 | 11.649 | 1.00 | 66.46 | B |
| 7239 | CD1 | TYR | B | 1458 | 10.034 | 11.463 | 11.356 | 1.00 | 67.99 | B |
| 7240 | CE1 | TYR | B | 1458 | 9.419 | 12.529 | 11.988 | 1.00 | 71.01 | B |
| 7241 | CD2 | TYR | B | 1458 | 8.601 | 9.894 | 12.587 | 1.00 | 66.33 | B |
| 7242 | CE2 | TYR | B | 1458 | 7.943 | 10.960 | 13.196 | 1.00 | 70.64 | B |
| 7243 | CZ | TYR | B | 1458 | 8.376 | 12.287 | 12.904 | 1.00 | 69.78 | B |
| 7244 | OH | TYR | B | 1458 | 7.745 | 13.386 | 13.472 | 1.00 | 70.03 | B |
| 7245 | C | TYR | B | 1458 | 12.301 | 9.377 | 12.489 | 1.00 | 59.69 | B |
| 7246 | O | TYR | B | 1458 | 13.240 | 9.604 | 11.787 | 1.00 | 60.39 | B |
| 7247 | N | ASN | B | 1459 | 12.122 | 9.947 | 13.656 | 1.00 | 56.36 | B |
| 7248 | CA | ASN | B | 1459 | 13.167 | 10.821 | 13.991 | 1.00 | 55.87 | B |
| 7249 | CB | ASN | B | 1459 | 12.782 | 12.258 | 13.648 | 1.00 | 55.60 | B |
| 7250 | CG | ASN | B | 1459 | 11.607 | 12.690 | 14.435 | 1.00 | 56.33 | B |
| 7251 | OD1 | ASN | B | 1459 | 10.799 | 11.861 | 14.907 | 1.00 | 58.79 | B |
| 7252 | ND2 | ASN | B | 1459 | 11.420 | 13.979 | 14.509 | 1.00 | 54.32 | B |
| 7253 | C | ASN | B | 1459 | 13.710 | 10.593 | 15.412 | 1.00 | 53.48 | B |
| 7254 | O | ASN | B | 1459 | 14.746 | 11.128 | 15.698 | 1.00 | 52.72 | B |
| 7255 | N | LYS | B | 1460 | 13.086 | 9.664 | 16.132 | 1.00 | 51.45 | B |
| 7256 | CA | LYS | B | 1460 | 13.393 | 9.303 | 17.462 | 1.00 | 51.74 | B |
| 7257 | CB | LYS | B | 1460 | 12.133 | 9.249 | 18.370 | 1.00 | 51.60 | B |
| 7258 | CG | LYS | B | 1460 | 11.194 | 10.519 | 18.585 | 1.00 | 55.18 | B |
| 7259 | CD | LYS | B | 1460 | 9.788 | 10.371 | 17.906 | 1.00 | 55.95 | B |
| 7260 | CE | LYS | B | 1460 | 8.819 | 11.516 | 18.181 | 1.00 | 52.32 | B |
| 7261 | NZ | LYS | B | 1460 | 7.272 | 11.181 | 17.989 | 1.00 | 48.55 | B |
| 7262 | C | LYS | B | 1460 | 14.057 | 7.942 | 17.645 | 1.00 | 53.06 | B |
| 7263 | O | LYS | B | 1460 | 13.865 | 6.946 | 16.810 | 1.00 | 53.05 | B |
| 7264 | N | VAL | B | 1461 | 14.680 | 7.829 | 18.846 | 1.00 | 51.75 | B |
| 7265 | CA | VAL | B | 1461 | 15.409 | 6.659 | 19.217 | 1.00 | 49.19 | B |
| 7266 | CB | VAL | B | 1461 | 16.834 | 6.994 | 19.078 | 1.00 | 49.92 | B |
| 7267 | CG1 | VAL | B | 1461 | 17.325 | 7.899 | 20.199 | 1.00 | 42.55 | B |
| 7268 | CG2 | VAL | B | 1461 | 17.741 | 5.706 | 18.753 | 1.00 | 50.60 | B |
| 7269 | C | VAL | B | 1461 | 15.095 | 6.478 | 20.664 | 1.00 | 50.75 | B |
| 7270 | O | VAL | B | 1461 | 14.927 | 7.418 | 21.399 | 1.00 | 52.10 | B |
| 7271 | N | ALA | B | 1462 | 14.972 | 5.246 | 21.099 | 1.00 | 50.56 | B |
| 7272 | CA | ALA | B | 1462 | 14.698 | 4.954 | 22.449 | 1.00 | 48.87 | B |
| 7273 | CB | ALA | B | 1462 | 13.565 | 4.056 | 22.505 | 1.00 | 46.24 | B |
| 7274 | C | ALA | B | 1462 | 16.034 | 4.345 | 23.058 | 1.00 | 49.38 | B |
| 7275 | O | ALA | B | 1462 | 16.922 | 3.872 | 22.312 | 1.00 | 50.01 | B |
| 7276 | N | ILE | B | 1463 | 16.222 | 4.388 | 24.363 | 1.00 | 47.94 | B |
| 7277 | CA | ILE | B | 1463 | 17.551 | 4.025 | 24.835 | 1.00 | 47.11 | B |
| 7278 | CB | ILE | B | 1463 | 18.344 | 5.225 | 25.292 | 1.00 | 48.94 | B |
| 7279 | CG2 | ILE | B | 1463 | 19.869 | 4.727 | 25.698 | 1.00 | 46.52 | B |
| 7280 | CG1 | ILE | B | 1463 | 18.143 | 6.423 | 24.269 | 1.00 | 51.05 | B |
| 7281 | CD1 | ILE | B | 1463 | 18.669 | 7.792 | 24.570 | 1.00 | 44.98 | B |
| 7282 | C | ILE | B | 1463 | 17.500 | 3.107 | 26.002 | 1.00 | 49.20 | B |
| 7283 | O | ILE | B | 1463 | 17.120 | 3.510 | 27.239 | 1.00 | 46.01 | B |
| 7284 | N | LYS | B | 1464 | 17.890 | 1.880 | 25.616 | 1.00 | 50.69 | B |
| 7285 | CA | LYS | B | 1464 | 17.625 | 0.582 | 26.446 | 1.00 | 53.41 | B |
| 7286 | CB | LYS | B | 1464 | 17.322 | −0.632 | 25.633 | 1.00 | 51.69 | B |
| 7287 | CG | LYS | B | 1464 | 16.964 | −1.783 | 26.441 | 1.00 | 58.03 | B |
| 7288 | CD | LYS | B | 1464 | 17.777 | −3.136 | 26.086 | 1.00 | 62.97 | B |
| 7289 | CE | LYS | B | 1464 | 17.049 | −4.179 | 25.146 | 1.00 | 49.51 | B |
| 7290 | NZ | LYS | B | 1464 | 16.141 | −4.963 | 25.999 | 1.00 | 49.33 | B |
| 7291 | C | LYS | B | 1464 | 18.905 | 0.355 | 27.147 | 1.00 | 53.63 | B |
| 7292 | O | LYS | B | 1464 | 19.927 | 0.572 | 26.466 | 1.00 | 53.71 | B |
| 7293 | N | VAL | B | 1465 | 18.808 | 0.142 | 28.475 | 1.00 | 53.24 | B |
| 7294 | CA | VAL | B | 1465 | 19.932 | −0.245 | 29.412 | 1.00 | 54.84 | B |
| 7295 | CB | VAL | B | 1465 | 21.052 | 0.860 | 29.725 | 1.00 | 55.07 | B |
| 7296 | CG1 | VAL | B | 1465 | 20.459 | 2.135 | 30.238 | 1.00 | 58.21 | B |
| 7297 | CG2 | VAL | B | 1465 | 22.193 | 0.400 | 30.639 | 1.00 | 49.92 | B |
| 7298 | C | VAL | B | 1465 | 19.322 | −0.918 | 30.632 | 1.00 | 54.16 | B |
| 7299 | O | VAL | B | 1465 | 18.307 | −0.502 | 31.116 | 1.00 | 53.64 | B |
| 7300 | N | GLY | B | 1466 | 19.862 | −2.088 | 30.952 | 1.00 | 54.33 | B |
| 7301 | CA | GLY | B | 1466 | 19.346 | −2.975 | 32.018 | 1.00 | 51.98 | B |
| 7302 | C | GLY | B | 1466 | 17.987 | −3.236 | 31.515 | 1.00 | 50.81 | B |
| 7303 | O | GLY | B | 1466 | 17.138 | −2.892 | 32.151 | 1.00 | 54.12 | B |
| 7304 | N | GLY | B | 1467 | 17.741 | −3.859 | 30.392 | 1.00 | 49.31 | B |
| 7305 | CA | GLY | B | 1467 | 16.405 | −4.170 | 30.013 | 1.00 | 47.50 | B |
| 7306 | C | GLY | B | 1467 | 15.390 | −3.147 | 30.417 | 1.00 | 50.17 | B |
| 7307 | O | GLY | B | 1467 | 14.223 | −3.503 | 30.576 | 1.00 | 51.96 | B |
| 7308 | N | ARG | B | 1468 | 15.739 | −1.903 | 30.736 | 1.00 | 51.17 | B |
| 7309 | CA | ARG | B | 1468 | 14.666 | −0.768 | 30.691 | 1.00 | 54.30 | B |
| 7310 | CB | ARG | B | 1468 | 14.583 | −0.132 | 32.061 | 1.00 | 51.82 | B |
| 7311 | CG | ARG | B | 1468 | 13.899 | −0.835 | 32.861 | 1.00 | 53.87 | B |
| 7312 | CD | ARG | B | 1468 | 14.857 | −1.336 | 34.022 | 1.00 | 66.93 | B |
| 7313 | NE | ARG | B | 1468 | 13.958 | −2.097 | 34.957 | 1.00 | 70.69 | B |
| 7314 | CZ | ARG | B | 1468 | 14.272 | −2.895 | 35.958 | 1.00 | 64.02 | B |
| 7315 | NH1 | ARG | B | 1468 | 15.529 | −3.133 | 36.239 | 1.00 | 62.58 | B |
| 7316 | NH2 | ARG | B | 1468 | 13.268 | −3.479 | 36.606 | 1.00 | 62.52 | B |
| 7317 | C | ARG | B | 1468 | 14.843 | 0.434 | 29.545 | 1.00 | 55.81 | B |
| 7318 | O | ARG | B | 1468 | 15.751 | 0.350 | 28.618 | 1.00 | 57.93 | B |
| 7319 | N | TYR | B | 1469 | 14.177 | 1.592 | 29.703 | 1.00 | 54.91 | B |
| 7320 | CA | TYR | B | 1469 | 14.512 | 2.722 | 28.806 | 1.00 | 54.21 | B |
| 7321 | CB | TYR | B | 1469 | 13.377 | 3.012 | 27.695 | 1.00 | 52.56 | B |
| 7322 | CG | TYR | B | 1469 | 13.315 | 1.875 | 26.689 | 1.00 | 51.66 | B |
| 7323 | CD1 | TYR | B | 1469 | 14.149 | 1.792 | 25.555 | 1.00 | 51.12 | B |
| 7324 | CE1 | TYR | B | 1469 | 13.983 | 0.560 | 24.597 | 1.00 | 50.58 | B |
| 7325 | CD2 | TYR | B | 1469 | 12.443 | 0.786 | 26.928 | 1.00 | 56.88 | B |
| 7326 | CE2 | TYR | B | 1469 | 12.345 | −0.340 | 26.108 | 1.00 | 54.54 | B |
| 7327 | CZ | TYR | B | 1469 | 13.127 | −0.461 | 24.956 | 1.00 | 54.25 | B |
| 7328 | OH | TYR | B | 1469 | 12.967 | −1.665 | 24.335 | 1.00 | 49.99 | B |
| 7329 | C | TYR | B | 1469 | 14.901 | 3.932 | 29.546 | 1.00 | 53.37 | B |
| 7330 | O | TYR | B | 1469 | 14.385 | 4.190 | 30.548 | 1.00 | 53.21 | B |
| 7331 | N | LEU | B | 1470 | 15.772 | 4.728 | 29.002 | 1.00 | 54.95 | B |
| 7332 | CA | LEU | B | 1470 | 15.991 | 5.994 | 29.638 | 1.00 | 58.05 | B |
| 7333 | CB | LEU | B | 1470 | 16.987 | 6.836 | 28.852 | 1.00 | 58.93 | B |
| 7334 | CG | LEU | B | 1470 | 18.469 | 7.020 | 29.233 | 1.00 | 61.91 | B |
| 7335 | CD1 | LEU | B | 1470 | 19.294 | 5.771 | 29.093 | 1.00 | 60.88 | B |
| 7336 | CD2 | LEU | B | 1470 | 19.060 | 8.081 | 28.373 | 1.00 | 59.27 | B |
| 7337 | C | LEU | B | 1470 | 14.774 | 6.751 | 29.463 | 1.00 | 58.65 | B |
| 7338 | O | LEU | B | 1470 | 14.487 | 6.984 | 28.348 | 1.00 | 59.27 | B |
| 7339 | N | LYS | B | 1471 | 14.116 | 7.172 | 30.576 | 1.00 | 60.28 | B |
| 7340 | CA | LYS | B | 1471 | 12.914 | 8.058 | 30.640 | 1.00 | 59.74 | B |
| 7341 | CB | LYS | B | 1471 | 11.674 | 7.155 | 30.853 | 1.00 | 57.21 | B |
| 7342 | CG | LYS | B | 1471 | 10.375 | 7.885 | 30.803 | 1.00 | 55.54 | B |
| 7343 | CD | LYS | B | 1471 | 9.354 | 7.011 | 31.569 | 1.00 | 56.04 | B |
| 7344 | CE | LYS | B | 1471 | 7.898 | 7.643 | 31.856 | 1.00 | 46.04 | B |
| 7345 | NZ | LYS | B | 1471 | 6.787 | 6.672 | 32.531 | 1.00 | 46.36 | B |
| 7346 | C | LYS | B | 1471 | 12.981 | 9.178 | 31.799 | 1.00 | 61.35 | B |
| 7347 | O | LYS | B | 1471 | 12.898 | 8.814 | 33.045 | 1.00 | 60.16 | B |
| 7348 | N | GLY | B | 1472 | 13.113 | 10.491 | 31.399 | 1.00 | 61.22 | B |
| 7349 | CA | GLY | B | 1472 | 13.028 | 11.632 | 32.330 | 1.00 | 62.05 | B |
| 7350 | C | GLY | B | 1472 | 11.801 | 11.644 | 33.239 | 1.00 | 64.71 | B |
| 7351 | O | GLY | B | 1472 | 10.750 | 11.989 | 32.711 | 1.00 | 70.04 | B |
| 7352 | N | ASP | B | 1473 | 11.874 | 11.301 | 34.548 | 1.00 | 63.13 | B |
| 7353 | CA | ASP | B | 1473 | 10.765 | 11.264 | 35.565 | 1.00 | 62.42 | B |
| 7354 | CB | ASP | B | 1473 | 11.220 | 10.377 | 36.711 | 1.00 | 61.90 | B |
| 7355 | CG | ASP | B | 1473 | 12.035 | 11.154 | 37.782 | 1.00 | 64.29 | B |
| 7356 | OD1 | ASP | B | 1473 | 13.099 | 11.766 | 37.455 | 1.00 | 66.79 | B |
| 7357 | OD2 | ASP | B | 1473 | 11.604 | 11.138 | 38.974 | 1.00 | 57.99 | B |
| 7358 | C | ASP | B | 1473 | 10.164 | 12.558 | 36.256 | 1.00 | 62.63 | B |
| 7359 | O | ASP | B | 1473 | 10.329 | 13.654 | 35.751 | 1.00 | 63.67 | B |
| 7360 | N | HIS | B | 1474 | 9.400 | 12.409 | 37.359 | 1.00 | 62.29 | B |
| 7361 | CA | HIS | B | 1474 | 9.087 | 13.550 | 38.288 | 1.00 | 63.94 | B |
| 7362 | CB | HIS | B | 1474 | 8.653 | 13.038 | 39.683 | 1.00 | 64.81 | B |
| 7363 | CG | HIS | B | 1474 | 7.566 | 12.006 | 39.661 | 1.00 | 68.36 | B |
| 7364 | CD2 | HIS | B | 1474 | 6.363 | 11.962 | 40.312 | 1.00 | 64.57 | B |
| 7365 | ND1 | HIS | B | 1474 | 7.659 | 10.826 | 38.913 | 1.00 | 70.49 | B |
| 7366 | CE1 | HIS | B | 1474 | 6.541 | 10.132 | 39.076 | 1.00 | 70.06 | B |
| 7367 | NE2 | HIS | B | 1474 | 5.737 | 10.804 | 39.909 | 1.00 | 63.39 | B |
| 7368 | C | HIS | B | 1474 | 10.333 | 14.505 | 38.530 | 1.00 | 64.13 | B |
| 7369 | O | HIS | B | 1474 | 11.478 | 14.048 | 38.832 | 1.00 | 61.98 | B |
| 7370 | N | ALA | B | 1475 | 10.109 | 15.821 | 38.367 | 1.00 | 64.45 | B |
| 7371 | CA | ALA | B | 1475 | 11.246 | 16.874 | 38.315 | 1.00 | 63.45 | B |
| 7372 | CB | ALA | B | 1475 | 11.968 | 17.016 | 39.671 | 1.00 | 62.92 | B |
| 7373 | C | ALA | B | 1475 | 12.278 | 16.731 | 37.157 | 1.00 | 62.14 | B |
| 7374 | O | ALA | B | 1475 | 13.073 | 17.695 | 36.962 | 1.00 | 62.08 | B |
| 7375 | N | GLY | B | 1476 | 12.267 | 15.586 | 36.404 | 1.00 | 59.08 | B |
| 7376 | CA | GLY | B | 1476 | 12.982 | 15.489 | 35.146 | 1.00 | 57.91 | B |
| 7377 | C | GLY | B | 1476 | 14.178 | 14.578 | 35.116 | 1.00 | 59.13 | B |
| 7378 | O | GLY | B | 1476 | 14.966 | 14.606 | 34.199 | 1.00 | 60.45 | B |
| 7379 | N | VAL | B | 1477 | 14.323 | 13.683 | 36.068 | 1.00 | 59.24 | B |
| 7380 | CA | VAL | B | 1477 | 15.606 | 13.027 | 36.203 | 1.00 | 57.80 | B |
| 7381 | CB | VAL | B | 1477 | 15.758 | 12.467 | 37.589 | 1.00 | 58.76 | B |
| 7382 | CG1 | VAL | B | 1477 | 16.975 | 11.321 | 37.735 | 1.00 | 56.85 | B |
| 7383 | CG2 | VAL | B | 1477 | 15.866 | 13.655 | 38.516 | 1.00 | 57.94 | B |
| 7384 | C | VAL | B | 1477 | 15.709 | 11.945 | 35.253 | 1.00 | 56.77 | B |
| 7385 | O | VAL | B | 1477 | 14.840 | 11.112 | 35.224 | 1.00 | 57.22 | B |
| 7386 | N | LEU | B | 1478 | 16.800 | 11.866 | 34.526 | 1.00 | 55.22 | B |
| 7387 | CA | LEU | B | 1478 | 16.884 | 10.737 | 33.638 | 1.00 | 55.04 | B |
| 7388 | CB | LEU | B | 1478 | 17.985 | 10.978 | 32.674 | 1.00 | 55.44 | B |
| 7389 | CG | LEU | B | 1478 | 17.857 | 10.187 | 31.391 | 1.00 | 56.59 | B |
| 7390 | CD1 | LEU | B | 1478 | 16.575 | 10.635 | 30.734 | 1.00 | 49.56 | B |
| 7391 | CD2 | LEU | B | 1478 | 19.142 | 10.522 | 30.554 | 1.00 | 52.63 | B |
| 7392 | C | LEU | B | 1478 | 16.995 | 9.329 | 34.282 | 1.00 | 55.91 | B |
| 7393 | O | LEU | B | 1478 | 18.103 | 8.920 | 34.705 | 1.00 | 58.31 | B |
| 7394 | N | LYS | B | 1479 | 15.875 | 8.609 | 34.431 | 1.00 | 53.49 | B |
| 7395 | CA | LYS | B | 1479 | 16.015 | 7.293 | 34.954 | 1.00 | 53.36 | B |
| 7396 | CB | LYS | B | 1479 | 15.176 | 7.097 | 36.173 | 1.00 | 52.59 | B |
| 7397 | CG | LYS | B | 1479 | 14.982 | 8.364 | 37.052 | 1.00 | 54.99 | B |
| 7398 | CD | LYS | B | 1479 | 14.537 | 8.040 | 38.497 | 1.00 | 49.56 | B |
| 7399 | CE | LYS | B | 1479 | 15.504 | 8.442 | 39.496 | 1.00 | 46.63 | B |
| 7400 | NZ | LYS | B | 1479 | 15.587 | 7.438 | 40.576 | 1.00 | 46.04 | B |
| 7401 | C | LYS | B | 1479 | 15.707 | 6.264 | 33.862 | 1.00 | 55.21 | B |
| 7402 | O | LYS | B | 1479 | 14.907 | 6.515 | 32.895 | 1.00 | 57.50 | B |
| 7403 | N | ALA | B | 1480 | 16.333 | 5.102 | 34.000 | 1.00 | 53.43 | B |
| 7404 | CA | ALA | B | 1480 | 16.122 | 4.022 | 33.067 | 1.00 | 53.10 | B |
| 7405 | CB | ALA | B | 1480 | 17.372 | 3.249 | 32.933 | 1.00 | 53.64 | B |
| 7406 | C | ALA | B | 1480 | 15.142 | 3.080 | 33.589 | 1.00 | 52.82 | B |
| 7407 | O | ALA | B | 1480 | 15.537 | 2.065 | 33.992 | 1.00 | 55.98 | B |
| 7408 | N | SER | B | 1481 | 13.874 | 3.369 | 33.568 | 1.00 | 53.48 | B |
| 7409 | CA | SER | B | 1481 | 12.863 | 2.566 | 34.242 | 1.00 | 55.45 | B |
| 7410 | CB | SER | B | 1481 | 12.401 | 3.322 | 35.488 | 1.00 | 55.69 | B |
| 7411 | OG | SER | B | 1481 | 11.961 | 4.646 | 35.003 | 1.00 | 58.38 | B |
| 7412 | C | SER | B | 1481 | 11.634 | 2.352 | 33.358 | 1.00 | 56.84 | B |
| 7413 | O | SER | B | 1481 | 10.879 | 1.425 | 33.662 | 1.00 | 59.65 | B |
| 7414 | N | ALA | B | 1482 | 11.431 | 3.141 | 32.294 | 1.00 | 56.64 | B |
| 7415 | CA | ALA | B | 1482 | 10.315 | 2.949 | 31.345 | 1.00 | 59.13 | B |
| 7416 | CB | ALA | B | 1482 | 10.544 | 3.706 | 30.119 | 1.00 | 59.98 | B |
| 7417 | C | ALA | B | 1482 | 10.138 | 1.559 | 30.907 | 1.00 | 60.45 | B |
| 7418 | O | ALA | B | 1482 | 11.188 | 0.940 | 30.594 | 1.00 | 62.40 | B |
| 7419 | N | GLU | B | 1483 | 8.894 | 1.030 | 30.788 | 1.00 | 60.61 | B |
| 7420 | CA | GLU | B | 1483 | 8.944 | −0.461 | 30.655 | 1.00 | 63.29 | B |
| 7421 | CB | GLU | B | 1483 | 7.883 | −1.333 | 31.341 | 1.00 | 63.32 | B |
| 7422 | CG | GLU | B | 1483 | 8.296 | −2.942 | 31.247 | 1.00 | 59.35 | B |
| 7423 | CD | GLU | B | 1483 | 9.265 | −3.240 | 32.425 | 1.00 | 57.98 | B |
| 7424 | OE1 | GLU | B | 1483 | 9.012 | −3.927 | 33.413 | 1.00 | 60.33 | B |
| 7425 | OE2 | GLU | B | 1483 | 10.324 | −2.684 | 32.419 | 1.00 | 57.13 | B |
| 7426 | C | GLU | B | 1483 | 9.039 | −0.927 | 29.270 | 1.00 | 63.19 | B |
| 7427 | O | GLU | B | 1483 | 9.633 | −2.019 | 28.985 | 1.00 | 62.34 | B |
| 7428 | N | THR | B | 1484 | 8.427 | −0.093 | 28.441 | 1.00 | 62.70 | B |
| 7429 | CA | THR | B | 1484 | 8.303 | −0.413 | 27.060 | 1.00 | 64.59 | B |
| 7430 | CB | THR | B | 1484 | 6.990 | −1.050 | 26.748 | 1.00 | 64.67 | B |
| 7431 | OG1 | THR | B | 1484 | 6.027 | −0.587 | 27.708 | 1.00 | 68.57 | B |
| 7432 | CG2 | THR | B | 1484 | 7.093 | −2.554 | 26.779 | 1.00 | 65.31 | B |
| 7433 | C | THR | B | 1484 | 8.166 | 0.868 | 26.385 | 1.00 | 65.19 | B |
| 7434 | O | THR | B | 1484 | 7.122 | 1.573 | 26.587 | 1.00 | 67.95 | B |
| 7435 | N | VAL | B | 1485 | 9.171 | 1.177 | 25.549 | 1.00 | 63.27 | B |
| 7436 | CA | VAL | B | 1485 | 9.001 | 2.201 | 24.522 | 1.00 | 58.53 | B |
| 7437 | CB | VAL | B | 1485 | 8.940 | 1.607 | 23.173 | 1.00 | 57.24 | B |
| 7438 | CG1 | VAL | B | 1485 | 9.851 | 2.377 | 22.386 | 1.00 | 57.26 | B |
| 7439 | CG2 | VAL | B | 1485 | 9.230 | −0.058 | 23.094 | 1.00 | 59.27 | B |
| 7440 | C | VAL | B | 1485 | 7.774 | 3.132 | 24.675 | 1.00 | 58.07 | B |
| 7441 | O | VAL | B | 1485 | 6.624 | 2.647 | 24.770 | 1.00 | 57.57 | B |
| 7442 | N | ASP | B | 1486 | 7.960 | 4.454 | 24.694 | 1.00 | 56.23 | B |
| 7443 | CA | ASP | B | 1486 | 6.737 | 5.288 | 24.507 | 1.00 | 55.55 | B |
| 7444 | CB | ASP | B | 1486 | 5.883 | 5.277 | 25.806 | 1.00 | 57.79 | B |
| 7445 | CG | ASP | B | 1486 | 6.697 | 5.809 | 27.107 | 1.00 | 55.70 | B |
| 7446 | OD1 | ASP | B | 1486 | 6.102 | 5.911 | 28.132 | 1.00 | 50.86 | B |
| 7447 | OD2 | ASP | B | 1486 | 7.920 | 6.065 | 27.055 | 1.00 | 55.56 | B |
| 7448 | C | ASP | B | 1486 | 7.407 | 6.627 | 24.448 | 1.00 | 55.42 | B |
| 7449 | O | ASP | B | 1486 | 8.681 | 6.576 | 24.633 | 1.00 | 54.87 | B |
| 7450 | N | PRO | B | 1487 | 6.571 | 7.747 | 24.410 | 1.00 | 51.92 | B |
| 7451 | CD | PRO | B | 1487 | 5.140 | 7.367 | 24.341 | 1.00 | 49.86 | B |
| 7452 | CA | PRO | B | 1487 | 6.740 | 9.216 | 24.453 | 1.00 | 53.23 | B |
| 7453 | CB | PRO | B | 1487 | 5.294 | 9.813 | 24.686 | 1.00 | 52.51 | B |
| 7454 | CG | PRO | B | 1487 | 4.314 | 8.739 | 24.461 | 1.00 | 48.36 | B |
| 7455 | C | PRO | B | 1487 | 7.682 | 9.800 | 25.502 | 1.00 | 53.98 | B |
| 7456 | O | PRO | B | 1487 | 8.476 | 10.639 | 25.145 | 1.00 | 56.24 | B |
| 7457 | N | ALA | B | 1488 | 7.617 | 9.329 | 26.742 | 1.00 | 53.47 | B |
| 7458 | CA | ALA | B | 1488 | 8.621 | 9.654 | 27.775 | 1.00 | 53.83 | B |
| 7459 | CB | ALA | B | 1488 | 8.128 | 9.080 | 29.214 | 1.00 | 51.24 | B |
| 7460 | C | ALA | B | 1488 | 10.019 | 9.105 | 27.420 | 1.00 | 53.64 | B |
| 7461 | O | ALA | B | 1488 | 10.910 | 9.806 | 27.180 | 1.00 | 54.12 | B |
| 7462 | N | SER | B | 1489 | 10.173 | 7.805 | 27.397 | 1.00 | 55.24 | B |
| 7463 | CA | SER | B | 1489 | 11.335 | 7.182 | 26.908 | 1.00 | 56.22 | B |
| 7464 | CB | SER | B | 1489 | 11.475 | 5.744 | 27.412 | 1.00 | 54.99 | B |
| 7465 | OG | SER | B | 1489 | 10.195 | 5.107 | 27.444 | 1.00 | 63.21 | B |
| 7466 | C | SER | B | 1489 | 11.458 | 7.302 | 25.389 | 1.00 | 55.71 | B |
| 7467 | O | SER | B | 1489 | 11.521 | 6.329 | 24.697 | 1.00 | 58.63 | B |
| 7468 | N | LEU | B | 1490 | 11.519 | 8.510 | 24.869 | 1.00 | 55.54 | B |
| 7469 | CA | LEU | B | 1490 | 12.016 | 8.712 | 23.490 | 1.00 | 53.60 | B |
| 7470 | CB | LEU | B | 1490 | 10.921 | 8.502 | 22.514 | 1.00 | 53.36 | B |
| 7471 | CG | LEU | B | 1490 | 10.431 | 7.352 | 21.697 | 1.00 | 51.34 | B |
| 7472 | CD1 | LEU | B | 1490 | 9.067 | 8.058 | 21.183 | 1.00 | 52.37 | B |
| 7473 | CD2 | LEU | B | 1490 | 11.383 | 7.075 | 20.480 | 1.00 | 51.94 | B |
| 7474 | C | LEU | B | 1490 | 12.400 | 10.184 | 23.287 | 1.00 | 53.65 | B |
| 7475 | O | LEU | B | 1490 | 11.915 | 11.100 | 24.076 | 1.00 | 51.62 | B |
| 7476 | N | TRP | B | 1491 | 13.214 | 10.361 | 22.223 | 1.00 | 51.38 | B |
| 7477 | CA | TRP | B | 1491 | 14.049 | 11.509 | 21.950 | 1.00 | 51.14 | B |
| 7478 | CB | TRP | B | 1491 | 15.419 | 11.202 | 22.626 | 1.00 | 50.85 | B |
| 7479 | CG | TRP | B | 1491 | 15.287 | 10.460 | 24.061 | 1.00 | 50.05 | B |
| 7480 | CD2 | TRP | B | 1491 | 14.865 | 11.084 | 25.382 | 1.00 | 50.02 | B |
| 7481 | CE2 | TRP | B | 1491 | 14.894 | 10.050 | 26.336 | 1.00 | 48.70 | B |
| 7482 | CE3 | TRP | B | 1491 | 14.539 | 12.422 | 25.823 | 1.00 | 47.12 | B |
| 7483 | CD1 | TRP | B | 1491 | 15.562 | 9.169 | 24.312 | 1.00 | 43.94 | B |
| 7484 | NE1 | TRP | B | 1491 | 15.319 | 8.912 | 25.647 | 1.00 | 49.67 | B |
| 7485 | CZ2 | TRP | B | 1491 | 14.580 | 10.273 | 27.724 | 1.00 | 44.77 | B |
| 7486 | CZ3 | TRP | B | 1491 | 14.160 | 12.635 | 27.262 | 1.00 | 46.30 | B |
| 7487 | CH2 | TRP | B | 1491 | 14.164 | 11.565 | 28.147 | 1.00 | 43.91 | B |
| 7488 | C | TRP | B | 1491 | 14.378 | 11.678 | 20.425 | 1.00 | 51.92 | B |
| 7489 | O | TRP | B | 1491 | 14.422 | 10.713 | 19.710 | 1.00 | 52.85 | B |
| 7490 | N | GLU | B | 1492 | 14.667 | 12.887 | 20.006 | 1.00 | 50.46 | B |
| 7491 | CA | GLU | B | 1492 | 15.290 | 13.190 | 18.799 | 1.00 | 51.35 | B |
| 7492 | CB | GLU | B | 1492 | 14.706 | 14.535 | 18.172 | 1.00 | 51.01 | B |
| 7493 | CG | GLU | B | 1492 | 13.121 | 14.587 | 18.104 | 1.00 | 50.74 | B |
| 7494 | CD | GLU | B | 1492 | 12.461 | 15.964 | 18.296 | 1.00 | 52.68 | B |
| 7495 | OE1 | GLU | B | 1492 | 13.156 | 17.023 | 18.118 | 1.00 | 49.77 | B |
| 7496 | OE2 | GLU | B | 1492 | 11.205 | 15.988 | 18.692 | 1.00 | 46.64 | B |
| 7497 | C | GLU | B | 1492 | 16.831 | 13.226 | 18.913 | 1.00 | 51.55 | B |
| 7498 | O | GLU | B | 1492 | 17.485 | 14.200 | 19.436 | 1.00 | 51.33 | B |
| 7499 | N | TYR | B | 1493 | 17.473 | 12.210 | 18.374 | 1.00 | 49.11 | B |
| 7500 | CA | TYR | B | 1493 | 18.896 | 12.433 | 18.069 | 1.00 | 47.50 | B |
| 7501 | CB | TYR | B | 1493 | 19.474 | 11.161 | 17.576 | 1.00 | 47.06 | B |
| 7502 | CG | TYR | B | 1493 | 18.712 | 10.498 | 16.487 | 1.00 | 45.62 | B |
| 7503 | CD1 | TYR | B | 1493 | 17.524 | 9.814 | 16.761 | 1.00 | 51.35 | B |
| 7504 | CE1 | TYR | B | 1493 | 16.831 | 9.135 | 15.745 | 1.00 | 51.24 | B |
| 7505 | CD2 | TYR | B | 1493 | 19.217 | 10.467 | 15.182 | 1.00 | 43.39 | B |
| 7506 | CE2 | TYR | B | 1493 | 18.601 | 9.788 | 14.193 | 1.00 | 39.85 | B |
| 7507 | CZ | TYR | B | 1493 | 17.430 | 9.130 | 14.430 | 1.00 | 47.69 | B |
| 7508 | OH | TYR | B | 1493 | 16.759 | 8.578 | 13.363 | 1.00 | 45.65 | B |
| 7509 | C | TYR | B | 1493 | 19.140 | 13.496 | 16.956 | 1.00 | 49.64 | B |
| 7510 | O | TYR | B | 1493 | 18.062 | 14.048 | 16.548 | 1.00 | 52.39 | B |
| 7511 | OXT | TYR | B | 1493 | 20.255 | 13.799 | 16.413 | 1.00 | 46.28 | B |
| 7512 | O18 | XYZ | C | 1 | −3.838 | −4.342 | 38.231 | 1.00 | 82.39 | C |
| 7513 | C17 | XYZ | C | 1 | −3.375 | −5.331 | 38.491 | 1.00 | 82.39 | C |
| 7514 | C16 | XYZ | C | 1 | −3.588 | −6.515 | 37.603 | 1.00 | 82.39 | C |
| 7515 | C15 | XYZ | C | 1 | −2.651 | −6.433 | 36.386 | 1.00 | 82.39 | C |
| 7516 | C14 | XYZ | C | 1 | −2.513 | −7.808 | 35.714 | 1.00 | 82.39 | C |
| 7517 | C13 | XYZ | C | 1 | −1.231 | −7.897 | 34.846 | 1.00 | 82.39 | C |
| 7518 | C12 | XYZ | C | 1 | −0.075 | −8.432 | 35.683 | 1.00 | 82.39 | C |
| 7519 | C11 | XYZ | C | 1 | 0.650 | −7.591 | 36.482 | 1.00 | 82.39 | C |
| 7520 | C8 | XYZ | C | 1 | 1.798 | −8.108 | 37.369 | 1.00 | 82.39 | C |
| 7521 | O9 | XYZ | C | 1 | 1.491 | −9.351 | 38.094 | 1.00 | 82.39 | C |
| 7522 | C10 | XYZ | C | 1 | 1.645 | −10.598 | 37.336 | 1.00 | 82.39 | C |
| 7523 | C6 | XYZ | C | 1 | 2.222 | −7.079 | 38.393 | 1.00 | 82.39 | C |
| 7524 | O7 | XYZ | C | 1 | 1.917 | −5.646 | 37.881 | 1.00 | 82.39 | C |
| 7525 | C4 | XYZ | C | 1 | 1.676 | −7.359 | 39.857 | 1.00 | 82.39 | C |
| 7526 | C5 | XYZ | C | 1 | 2.510 | −6.519 | 40.866 | 1.00 | 82.39 | C |
| 7527 | C3 | XYZ | C | 1 | 0.210 | −6.909 | 40.120 | 1.00 | 82.39 | C |
| 7528 | C2 | XYZ | C | 1 | −0.963 | −7.562 | 39.863 | 1.00 | 82.39 | C |
| 7529 | C1 | XYZ | C | 1 | −1.066 | −8.931 | 39.244 | 1.00 | 82.39 | C |
| 7530 | C20 | XYZ | C | 1 | −2.312 | −6.860 | 40.262 | 1.00 | 82.39 | C |
| 7531 | C19 | XYZ | C | 1 | −2.457 | −5.395 | 39.707 | 1.00 | 82.39 | C |
| 7532 | O | HOH | W | 1 | −22.823 | 3.020 | 18.627 | 1.00 | 65.23 | W |
| 7533 | O | HOH | W | 2 | −8.627 | 8.401 | 44.702 | 1.00 | 63.79 | W |
| 7534 | O | HOH | W | 3 | −19.265 | 21.432 | 27.751 | 1.00 | 75.34 | W |
| 7535 | O | HOH | W | 4 | 33.378 | 31.211 | 8.989 | 1.00 | 75.04 | W |
| 7536 | O | HOH | W | 5 | −14.549 | −31.394 | 24.843 | 1.00 | 60.50 | W |
| 7537 | O | HOH | W | 6 | 33.114 | −11.026 | 13.023 | 1.00 | 59.44 | W |
| 7538 | O | HOH | W | 7 | −33.460 | −30.942 | 12.212 | 1.00 | 64.45 | W |
| 7539 | O | HOH | W | 8 | 22.793 | −15.306 | −2.398 | 1.00 | 57.16 | W |
| 7540 | O | HOH | W | 9 | −30.598 | 25.384 | 28.884 | 1.00 | 67.50 | W |
| 7541 | O | HOH | W | 10 | 11.971 | 31.978 | 1.485 | 1.00 | 78.33 | W |
| 7542 | O | HOH | W | 11 | 14.437 | 4.260 | 40.537 | 1.00 | 84.53 | W |
| 7543 | O | HOH | W | 12 | −0.602 | 40.561 | 5.615 | 1.00 | 54.70 | W |
| 7544 | O | HOH | W | 13 | −37.458 | 25.807 | 14.873 | 1.00 | 80.19 | W |
| 7545 | O | HOH | W | 14 | −12.522 | −22.963 | 24.635 | 1.00 | 54.03 | W |
| 7546 | O | HOH | W | 15 | −12.965 | 21.504 | 22.541 | 1.00 | 62.67 | W |
| 7547 | O | HOH | W | 16 | −29.898 | 28.147 | 54.991 | 1.00 | 53.66 | W |
| 7548 | O | HOH | W | 17 | −12.464 | 16.104 | 27.398 | 1.00 | 65.82 | W |
| 7549 | O | HOH | W | 18 | 41.614 | 28.299 | 19.175 | 1.00 | 67.17 | W |
| 7550 | O | HOH | W | 19 | 30.094 | 6.672 | 16.982 | 1.00 | 57.62 | W |
| 7551 | O | HOH | W | 20 | 18.827 | 29.361 | −1.024 | 1.00 | 94.90 | W |
| 7552 | O | HOH | W | 21 | 17.291 | 30.937 | −5.675 | 1.00 | 72.11 | W |
| 7553 | O | HOH | W | 22 | 26.398 | −20.845 | 2.236 | 1.00 | 76.75 | W |
| 7554 | O | HOH | W | 23 | 17.735 | 47.299 | 22.787 | 1.00 | 67.73 | W |
| 7555 | O | HOH | W | 24 | 15.126 | −6.940 | 27.558 | 1.00 | 62.60 | W |
| 7556 | O | HOH | W | 25 | −18.630 | 24.444 | 58.518 | 1.00 | 67.49 | W |
| 7557 | O | HOH | W | 26 | 4.167 | −10.720 | 3.052 | 1.00 | 80.75 | W |
| 7558 | O | HOH | W | 27 | −26.145 | −9.011 | 32.901 | 1.00 | 66.11 | W |
| 7559 | O | HOH | W | 28 | −27.515 | 8.571 | 37.231 | 1.00 | 77.41 | W |
| 7560 | O | HOH | W | 29 | 7.394 | −4.900 | 9.720 | 1.00 | 50.67 | W |
| 7561 | O | HOH | W | 30 | −21.185 | 3.572 | 47.583 | 1.00 | 56.13 | W |
| 7562 | O | HOH | W | 31 | −13.310 | 13.584 | 27.184 | 1.00 | 77.62 | W |
| 7563 | O | HOH | W | 32 | 31.704 | 1.032 | 10.546 | 1.00 | 54.63 | W |
| 7564 | O | HOH | W | 33 | −16.045 | 2.583 | 46.242 | 1.00 | 81.79 | W |
| 7565 | O | HOH | W | 34 | −5.072 | 44.351 | 26.027 | 1.00 | 66.82 | W |
| 7566 | O | HOH | W | 35 | −25.935 | 32.174 | 42.499 | 1.00 | 66.94 | W |
| 7567 | O | HOH | W | 36 | −8.888 | 0.244 | 39.543 | 1.00 | 73.20 | W |
| 7568 | O | HOH | W | 37 | −36.958 | 0.231 | 13.776 | 1.00 | 74.47 | W |
| 7569 | O | HOH | W | 38 | 37.355 | 37.679 | 10.419 | 1.00 | 97.11 | W |
| 7570 | O | HOH | W | 39 | 27.522 | 13.270 | 11.803 | 1.00 | 91.10 | W |
| 7571 | O | HOH | W | 40 | 32.761 | −2.760 | 12.373 | 1.00 | 53.44 | W |
| 7572 | O | HOH | W | 41 | −33.563 | 3.380 | 33.650 | 1.00 | 75.63 | W |
| 7573 | O | HOH | W | 42 | 8.406 | 10.941 | 7.956 | 1.00 | 59.61 | W |
| 7574 | O | HOH | W | 43 | −25.269 | 35.823 | 49.155 | 1.00 | 93.03 | W |
| 7575 | O | HOH | W | 44 | 11.064 | 12.373 | 30.025 | 1.00 | 78.73 | W |
| 7576 | O | HOH | W | 45 | 14.300 | 0.448 | 1.401 | 1.00 | 74.34 | W |
| 7577 | O | HOH | W | 46 | 22.689 | 33.320 | 6.116 | 1.00 | 99.20 | W |
| 7578 | O | HOH | W | 47 | 19.755 | −9.006 | −0.421 | 1.00 | 74.28 | W |
| 7579 | O | HOH | W | 48 | −15.215 | 30.874 | 30.724 | 1.00 | 90.34 | W |
| 7580 | O | HOH | W | 49 | 2.226 | −11.036 | 33.149 | 1.00 | 73.86 | W |
| 7581 | O | HOH | W | 50 | −30.305 | 4.027 | 8.547 | 1.00 | 66.08 | W |
| 7582 | O | HOH | W | 51 | 8.083 | 9.384 | 4.015 | 1.00 | 87.70 | W |
| 7583 | O | HOH | W | 52 | 9.847 | 19.520 | 25.157 | 1.00 | 68.60 | W |
| 7584 | O | HOH | W | 53 | −4.076 | 8.424 | 28.306 | 1.00 | 62.71 | W |
| 7585 | O | HOH | W | 54 | −13.137 | 36.123 | 37.081 | 1.00 | 79.64 | W |
| 7586 | O | HOH | W | 55 | 2.591 | 45.059 | 28.074 | 1.00 | 97.38 | W |
| 7587 | O | HOH | W | 56 | 32.996 | 11.321 | 4.805 | 1.00 | 51.94 | W |
| 7588 | O | HOH | W | 57 | −14.588 | 29.251 | 52.453 | 1.00 | 60.64 | W |
| 7589 | O | HOH | W | 58 | −10.962 | 24.049 | 31.004 | 1.00 | 83.64 | W |
| 7590 | O | HOH | W | 59 | −19.370 | 22.081 | 19.330 | 1.00 | 71.94 | W |
| END | | | | | | | | | | |

### BIBLIOGRAPHY

1. Adams, Curr Opin Cell Biol 16, 590 (October, 2004).
2. Aslakson, C. J., and Miller, F. R. (1992) Selective events in the metastatic process defined by analysis of the sequential dissemination of subpopulations of a mouse mammary tumor. Cancer Res 52, 1399-405.
3. Aznavoorian et al., Cancer 71, 1368 (Feb. 15, 1993).
4. Bryan et al., J Mol Biol 125, 207 (Oct. 25, 1978).
5. Cant et al., Genetics 143, 249 (May, 1996).
6. Cant et al., J Cell Biol 125, 369 (April, 1994).
7. Cao et al., Int J Gynecol Pathol 24, 67 (Jan., 2005).
8. Chen et al., Histopathology 51, 173 (August, 2007).
9. Christofori, Nature 441, 444 (May 25, 2006).
10. Condeelis et al., Annu Rev Cell Dev Biol 21, 695 (2005).
11. Doane et al., Oncogene 25, 3994 (Jun. 29, 2006).
12. Fidler, Nat Rev Cancer 3, 453 (June, 2003).
13. Gaul et al., J Am Chem Soc 126, 11326 (Sep. 15, 2004).
14. Grothey et al., Br J Cancer 83, 870 (October, 2000).
15. Hanahan et al., Cell 100, 57 (Jan. 7, 2000).
16. Hashimoto et al., Int J Biochem Cell Biol 37, 1787 (September, 2005).
17. Hashimoto et al., Oncology 67, 262 (2004).
18. Hu, W., McCrea, P. D., Deavers, M., Kavanagh, J. J., Kudelka, A. P., and Verschraegen, C. F. (2000) Increased expression of fascin, motility associated protein, in cell cultures derived from ovarian cancer and in borderline and carcinomatous ovarian tumors. Clin Exp Metastasis 18, 83-8.
19. Jaffe et al., Annu Rev Cell Dev Biol 21, 247 (2005).
20. Maitra et al., Am J Clin Pathol 118, 52 (July, 2002).
21. Matsudaira, Semin Cell Biol 5, 165 (June, 1994).
22. Minn et al., J Clin Invest 115, 44 (January, 2005).
23. Minn et al., Nature 436, 518 (Jul. 28, 2005).
24. Mogilner et al., Biophys J 89, 782 (August, 2005).
25. Nakae et al., J Antibiot (Tokyo) 53, 1130 (October, 2000).
26. Nakae et al., J Antibiot (Tokyo) 53, 1228 (October, 2000).
27. Nakamura et al., J Antibiot (Tokyo) 55, 442 (April, 2002).
28. Njardarson et al., J Am Chem Soc 126, 1038 (Feb. 4, 2004).
29. Ono et al., J Biol Chem 272, 2527 (Jan. 24, 1997).
30. Otto et al., Cell 17, 285 (June, 1979).
31. Otto, Curr Opin Cell Biol 6, 105 (February, 1994).
32. Partin et al., Proc Natl Acad Sci USA 86, 1254 (February, 1989).
33. Pelosi et al., Lung Cancer 42, 203 (November, 2003).
34. Pelosi, G., Pasini, F., Fraggetta, F., Pastorino, U., Iannucci, A., Maisonneuve, P., Arrigoni, G., De Manzoni, G., Bresaola, E., and Viale, G. (2003) Independent value of fascin immunoreactivity for predicting lymph node metastases in typical and atypical pulmonary carcinoids. Lung Cancer 42, 203-13.
35. Ponomarev et al., Eur J Nucl Med Mol Imaging 31, 740 (May, 2004).
36. Pulaski, B. A., and Ostrand-Rosenberg, S. (1998) Reduction of established spontaneous mammary carcinoma metastases following immunotherapy with major histocompatibility complex class II and B7.1 cell-based tumor vaccines. Cancer Res 58, 1486-93.
37. Ridley, A. J., Schwartz, M. A., Burridge, K., Firtel, R. A., Ginsberg, M. H., Borisy, G., Parsons, J. T., and Horwitz, A. R. (2003) Cell migration: integrating signals from front to back. Science 302, 1704-9.
38. Rodriguez-Pinilla et al., Clin Cancer Res 12, 1533 (Mar. 1, 2006).
39. Shan et al., Dev Cell 10, 707 (June, 2006).
40. Shan et al., Proc Natl Acad Sci USA 102, 3772 (Mar. 8, 2005).
41. Takemoto et al., J Antibiot (Tokyo) 54, 1104 (December, 2001).
42. Tilney et al., J Cell Biol 143, 121 (Oct. 5, 1998).
43. van de Vijver et al., N Engl J Med 347, 1999 (Dec. 19, 2002).
44. van 't Veer et al., Nature 415, 530 (Jan. 31, 2002).
45. Vignjevic et al., J Cell Biol 174, 863 (Sep. 11, 2006).
46. Weiss, Cancer Metastasis Rev 19, I (2000).
47. Woo et al., J Antibiot (Tokyo) 55, 141 (February, 2002).
48. Yamashiro-Matsumura et al., J Biol Chem 260, 5087 (Apr. 25, 1985).
49. Yang et al., J Biol Chem 280, 27130 (Jul. 22, 2005).
50. Yoder et al., Clin Cancer Res 11, 186 (Jan. 1, 2005).
51. Zigeuner et al., Urology 68, 518 (September, 2006).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method of inhibiting fascin expression and/or activity, comprising administering an effective amount of a fascin inhibitor to a cell expressing fascin to thereby inhibit the fascin expression or activity in the cell.

2. The method of claim 1, wherein the fascin inhibitor comprises an inhibitory nucleic acid that binds specifically to a fascin RNA or DNA consisting of SEQ ID NO:2, 4, 6 or 8, a small molecule, a fascin polypeptide fragment, or an antibody that binds specifically to fascin.

3. The method of claim 2, wherein the inhibitory nucleic acid is an RNA or DNA consisting of any of SEQ ID NOs:13-62.

4. The method of claim 3, wherein the inhibitory nucleic acid is administered by administering an expression vector comprising an expression cassette that directs the expression of the inhibitory nucleic acid.

5. The method of claim 2, wherein the antibody blocks actin binding to a fascin actin-binding site or binds specifically to a fascin actin-binding site.

6. The method of claim 5, wherein the fascin actin-binding site comprises any of fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

7. The method of claim 5, wherein the fascin actin-binding site comprises any of fascin amino acids His392, Glu391, Ala488, Lys471, His474 and Asp473.

8. The method of claim 5, wherein the antibody blocks actin binding to one or both of fascin amino acids His392 and His474 when bound to fascin protein.

9. The method of claim 5, wherein the antibody binds to one or both of fascin amino acids His392 and His474 when bound to fascin protein.

10. The method of claim 1, wherein the fascin inhibitor is a compound of formula I: 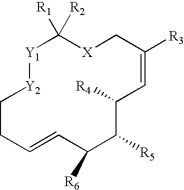
wherein:
X is CH, N, NH or O;
R₁ is OH, CZ₃ or R₁ and R₂ together are —C═O, wherein Z is halo;
R₂ is OH, CZ₃ or R₁ and R₂ together are —C═O, wherein Z is halo;
R₃ is H or lower alkyl;
R₄ is H or lower alkyl;
R₅ is OH;
R₆ is alkyloxy;
Y₁ and Y₂ are separately —CH₂— or Y₁ and Y₂ together form —C═C—
or a pharmaceutically acceptable salt thereof.

11. The method of claim 10, wherein the compound is any one of the following compounds, or a combination thereof:

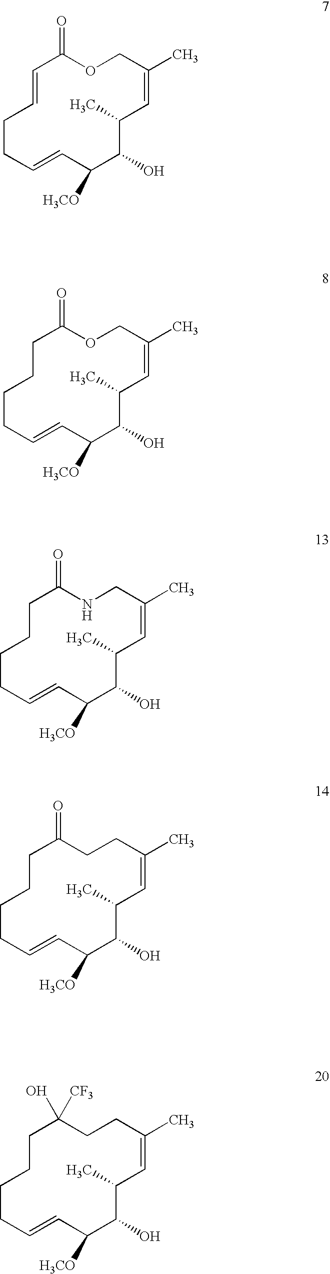

12. The method of claim 2, wherein the fascin polypeptide fragment comprises fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

13. The method of claim 2, wherein the fascin polypeptide fragment comprises fascin amino acids His392, Glu391, Ala488, Lys471, His474 and Asp473.

14. The method of claim 12, wherein the fascin polypeptide fragment consists of fascin amino acids 259 through 493.

15. The method of claim 2, wherein the fascin polypeptide fragment consists of SEQ ID NO:9, 10 and/or 12

16. The method of claim 1, wherein the cell is in an animal.

17. The method of claim 16, wherein the animal is a human.

18. The method of claim 17, wherein the human suffers from a disease or condition.

19. The method of claim 18, wherein the disease or condition is a metastatic cancer, a neuronal disorder, neuronal degeneration, an inflammatory condition, a viral infection, a bacterial infection, lymphoid hyperplasia, Hodgkin's disease or ischemia-related tissue damage.

20. The method of claim 19, wherein the cancer is a carcinoma, lymphoma, sarcoma, melanoma, astrocytoma, mesothelioma cells, ovarian carcinoma, colon carcinoma, pancreatic carcinoma, esophageal carcinoma, stomach carcinoma, lung carcinoma, urinary carcinoma, bladder carcinoma, breast cancer, gastric cancer, leukemia, lung cancer, colon cancer, central nervous system cancer, melanoma, ovarian cancer, renal cancer or prostate cancer.

21. A method of identifying an inhibitor of fascin, comprising:
a) contacting at least one protein or peptide having a fascin sequence with at least one test agent for a sufficient time to allow the components to interact; and
b) determining whether binding between the at least one protein or peptide having a fascin sequence and the test agent has occurred,
wherein binding between the at least one protein or peptide having a fascin sequence and test agent is indicative that the test agent is an inhibitor of cancer metastasis.

22. The method of claim 21, wherein the test agent blocks actin binding to a fascin actin-binding site or binds to a fascin actin-binding site.

23. The method of claim 21, wherein the fascin actin-binding site comprises fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

24. The method of claim 21, wherein the fascin actin-binding site comprises fascin amino acids His392, Glu391, Ala488, Lys471, His474 and Asp473.

25. The method of claim 21, wherein the test agent blocks actin binding to one or both of fascin amino acids His392 and His474 when bound to fascin protein.

26. The method of claim 21, wherein the test agent binds to one or both of fascin amino acids His392 and His474 when bound to fascin protein.

27. The method of claim 21, further comprising determining the binding constant of the test agent for fascin.

28. The method of claim 21, further comprising determining whether the test agent inhibits fascin-mediated actin bundle formation.

29. The method of claim 28, wherein the actin is F-actin.

30. A method for identifying an inhibitor of fascin, comprising:
a) generating a three-dimensional structural image of a fascin binding site from fascin atomic coordinates for fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, according to Table 2, ± a root mean square deviation from the backbone atoms of said amino acids of not more than 1.5 angstroms; and
b) designing or selecting a potential inhibitor to reside within the fascin binding site to thereby identify an inhibitor of fascin.

31. A method for identifying an inhibitor of fascin, comprising:
a) generating a three-dimensional structural image of a fascin binding site from fascin atomic coordinates for fascin amino acids His392, Glu391, Ala488, Lys471, His 474 and Asp473 according to Table 2, ± a root mean square deviation from the backbone atoms of said amino acids of not more than 1.5 angstroms; and
b) designing or selecting a potential inhibitor to reside within the fascin binding site to thereby identify an inhibitor of fascin.

32. The method of claim 30, further comprising synthesizing or obtaining the potential inhibitor, contacting the potential inhibitor with fascin, and ascertaining whether the potential inhibitor binds to fascin.

33. The method of claim 30, wherein the potential inhibitor is no larger than about eight (8) angstroms by about ten (10) angstroms by about ten (10) angstroms.

34. The method of claim 30, wherein the method is performed using a computer system comprising the fascin atomic coordinates as a data set.

35. The method of claim 1, wherein the inhibitor of fascin is an inhibitor of metastatic cancer.

36. A machine readable storage medium, comprising fascin atomic coordinates of Table 2.

37. The machine readable storage medium of claim 36, comprising fascin atomic coordinates for fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250, according to Table 2, ± a root mean square deviation from the backbone atoms of said amino acids of not more than 1.5 angstroms.

38. The machine readable storage medium of claim 36, comprising fascin atomic coordinates for fascin amino acids His392, Glu391, Ala488, Lys471, His474 and Asp473 according to Table 2, ± a root mean square deviation from the backbone atoms of said amino acids of not more than 1.5 angstroms.

39. A fascin inhibitor comprising an inhibitory nucleic acid that binds specifically to a fascin RNA or DNA consisting of SEQ ID NO:2, 4, 6 or 8, a small molecule, a fascin polypeptide fragment, or an antibody that binds specifically to fascin.

40. The fascin inhibitor of claim 39, wherein the inhibitory nucleic acid is an RNA or DNA consisting of any of SEQ ID NOs:13-62.

41. The fascin inhibitor of claim 39, wherein the inhibitory nucleic acid is expressed in an expression vector comprising an expression cassette that directs the expression of a fascin inhibitory nucleic acid.

42. The fascin inhibitor of claim 39, wherein the antibody binds specifically to a fascin actin-binding site, or blocks actin-binding to a fascin actin-binding site, wherein the actin-binding site comprises fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

43. The fascin inhibitor of claim 42, wherein the antibody binds specifically to a fascin actin-binding site, or blocks actin-binding to a fascin actin-binding site, wherein the actin-binding site comprises fascin amino acids His392, Glu391, Ala488, Lys471, His474 and Asp473.

44. The fascin inhibitor of claim 39, wherein the antibody was generated using a polypeptide with a sequence that includes fascin amino acids 259 through 493.

45. The fascin inhibitor of claim 39, wherein the antibody was generated using a polypeptide with SEQ ID NO:9, 10 and/or 12.

46. The fascin inhibitor of claim 39, wherein the fascin polypeptide fragment comprises fascin amino acids Thr326, Ser328, Ser329, Lys 330, Asn331, Ser333, Arg276, Gln 277, Met279, Asp286, Glu287, Gln291, Thr320, Thr318, Lys313, Thr311, Gln362, Asn360, Lys359, Asp168, Pro159, Arg151, Lys150, Arg149, Arg197, Arg201, Glu207, Glu227, Ser237, Pro236, Lys241, Lys247, and Lys250.

47. The fascin inhibitor of claim 39, wherein the fascin polypeptide fragment comprises fascin amino acids His392, Glu391, Ala488, Lys471, His474 and Asp473.

48. The fascin inhibitor of claim 39, wherein the fascin polypeptide fragment consists of fascin amino acids 259 through 493.

49. The fascin inhibitor of claim 39, wherein the fascin polypeptide fragment consists of SEQ ID NO:9, 10 and/or 12

50. A method of treating or inhibiting metastatic cancer in a patient, comprising administering to the patient, the fascin inhibitor of claim 39.

51-53. (canceled)


SEQUENCE LISTING (66 sequences)

SEQ ID NO: 1 - protein, 493 aa, Homo sapiens
MTANGTAEAVQIQFGLINCGNKYLTAEAFGFKVNASASSLKKKQIWTLEQPPDEAGSAAVCLRSHLGRYLAADKDGNVTCEREVPGPDCRFLIVAHDDGRWSLQSEAHRRYFGGTEDRLSCFAQTVSPAEKWSVHIAMHPQVNIYSVTRKRYAHLSARPADEIAVDRDVPWGVDSLITLAFQDQRYSVQTADHRFLRHDGRLVARPEPATGYTLEFRSGKVAFRDCEGRYLAPSGPSGTLKAGKATKVGKDELFALEQSCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 2 - DNA, 2800 bp, Homo sapiens
gctgcggagggtgcgtgcgggccgcggcagccgaacaaaggagcaggggcgccgccgcagggacccgccacccacctcccggggccgcgcagcggcctctcgtctactgccaccatgaccgccaacggcacagccgaggcggtgcagatccagttcggcctcatcaactgcggcaacaagtacctgacggccgaggcgttcgggttcaaggtgaacgcgtccgccagcagcctgaagaagaagcagatctggacgctggagcagccccctgacgaggcgggcagcgcggccgtgtgcctgcgcagccacctgggccgctacctggcggcggacaaggacggcaacgtgacctgcgagcgcgaggtgcccggtcccgactgccgtttcctcatcgtggcgcacgacgacggtcgctggtcgctgcagtccgaggcgcaccggcgctacttcggcggcaccgaggaccgcctgtcctgcttcgcgcagacggtgtcccccgccgagaagtggagcgtgcacatcgccatgcaccctcaggtcaacatctacagcgtcacccgtaagcgctacgcgcacctgagcgcgcggccggccgacgagatcgccgtggaccgcgacgtgccctggggcgtcgactcgctcatcaccctcgccttccaggaccagcgctacagcgtgcagaccgccgaccaccgcttcctgcgccacgacgggcgcctggtggcgcgccccgagccggccactggctacacgctggagttccgctccggcaaggtggccttccgcgactgcgagggccgttacctggcgccgtcggggcccagcggcacgctcaaggcgggcaaggccaccaaggtgggcaaggacgagctctttgctctggagcagagctgcgcccaggtcgtgctgcaggcggccaacgagaggaacgtgtccacgcgccagggtatggacctgtctgccaatcaggacgaggagaccgaccaggagaccttccagctggagatcgaccgcgacaccaaaaagtgtgccttccgtacccacacgggcaagtactggacgctgacggccaccgggggcgtgcagtccaccgcctccagcaagaatgccagctgctactttgacatcgagtggcgtgaccggcgcatcacactgagggcgtccaatggcaagtttgtgacctccaagaagaatgggcagctggccgcctcggtggagacagcaggggactcagagctcttcctcatgaagctcatcaaccgccccatcatcgtgttccgcggggagcatggcttcatcggctgccgcaaggtcacgggcaccctggacgccaaccgctccagctatgacgtcttccagctggagttcaacgatggcgcctacaacatcaaagactccacaggcaaatactggacggtgggcagtgactccgtggtcaccagcagcggcgacactcctgtggacttcttcttcgagttctgcgactataacaaggtggccatcaaggtgggcgggcgctacctgaagggcgaccacgcaggcgtcctgaaggcctcggcggaaaccgtggaccccgcctcgctctgggagtactagggccggcccgtccttccccgcccctgcccacatggcggctcctgccaaccctccctgctaaccccttctccgccaggtgggctccagggcgggaggcaagcccccttgcctttcaaactggaaaccccagagaaaacggtgcccccacctgtcgcccctatggactccccactctcccctccgcccgggttccctactcccctcgggtcagcggctgcggcctggccctgggagggatttcagatgcccctgccctcttgtctgccacggggcgagtctggcacctctttcttctgacctcagacggctctgagccttatttctctggaagcggctaagggacggttgggggctgggagccctgggcgtgtagtgtaactggaatcttttgcctctcccagccacctcctcccagccccccaggagagctgggcacatgtcccaagcctgtcagtggccctccctggtgcactgtccccgaaacccctgcttgggaagggaagctgtcgggtgggctaggactgacccttgtggtgtttttttgggtggtggctggaaacagcccctctcccacgtggcagaggctcagcctggctcccttccctggagcggcagggcgtgacggccacagggtctgcccgctgcacgttctgccaaggtggtggtggcgggcgggtaggggtgtgggggccgtcttcctcctgtctctttcctttcaccctagcctgactggaagcagaaaatgaccaaatcagtattttttttaatgaaatattattgctggaggcgtcccaggcaagcctggctgtagtagcgagtgatctggcggggggcgtctcagcaccctccccagggggtgcatctcagccccctctttccgtccttcccgtccagccccagccctgggcctgggctgccgacacctgggccagagcccctgctgtgattggtgctccctgggcctcccgggtggatgaagccaggcgtcgccccctccgggagccctggggtgagccgccggggccccccctgctgccagcctcccccgtccccaacatgcatctcactctgggtgtcttggtcttttattttttgtaagtgtcatttgtataactctaaacgcccatgatagtagcttcaaactggaaatagcgaaataaaataactcagtctgcagccccaaaaaaaaaaaaaaaaaaaaaaa

SEQ ID NO: 3 - protein, 516 aa, Homo sapiens
MPTNGLHQVLKIQFGLVNDTDRYLTAESFGFKVNASAPSLKRKQTWVLEPDPGQGTAVLLRSSHLGRYLSAEEDGRVACEAEQPGRDCRFLVLPQPDGRWVLRSEPHGRFFGGTEDQLSCFATAVSPAELWTVHLAIHPQAHLLSVSRRRYVHLCPREDEMAADGDKPWGVDALLTLIFRSRRYCLKSCDSRYLRSDGRLVWEPEPRACYTLEFKAGKLAFKDCDGHYLAPVGPAGTLKAGRNTRPGKDELFDLEESHPQVVLVAANHRYVSVRQGVNVSANQDDELDHETFLMQIDQETKKCTFYSSTGGYWTLVTHGGIHATATQVSANTMFEMEWRGRRVALKASNGRYVCMKKNGQLAAISDFVGPPPRPAWTGKVAGGAAQQTLSPPGKDEEFTLKLINRPILVLRGLDGFVCHHRGSNQLDTNRSVYDVFHLSFSDGAYRIRGRDGGFWYTGSHGSVCSDGERAEDFVFEFRERGRLAIRARSGKYLRGGASGLLRADADAPAGTALWEY

SEQ ID NO: 4 - DNA, 1762 bp, Homo sapiens
gcaggcagggggttcgtgacgccggctgggtctgggggctgtgggccagccgagccgacccgggcttctgggggaccgcgggggccgtgagcactcagagggtgcatcccaggcccctccggggacccggccagcctgaagatgccgacgaacggcctgcaccaggtgctgaagatccagtttggcctcgtcaacgacactgaccgctacctgacagctgagagcttcggcttcaaggtcaatgcctcggcacccagcctcaagaggaagcagacctgggtgctggaacccgacccaggacaaggcacggctgtgctgctccgcagcagccacctgggccgctacctgtcggcagaagaggacgggcgcgtggcctgtgaggcagagcagccgggccgtgactgccgcttcctggtcctgccgcagccagatgggcgctgggtgctgcggtccgagccgcacggccgcttcttcggaggcaccgaggaccagctgtcctgcttcgccacagccgtttccccggccgagctgtggaccgtgcacctggccatccacccgcaggcccacctgctgagcgtgagccggcggcgctacgtgcacctgtgcccgcgggaggacgagatggccgcagacggagacaagccctggggcgtggacgccctcctcaccctcatcttccggagccgacggtactgcctcaagtcctgtgacagccgctacctgcgcagcgacggccgtctggtctgggagcctgagccccgtgcctgctacacgctggagttcaaggcgggcaagctggccttcaaggactgcgacggccactacctggcacccgtggggcccgcaggcaccctcaaggccggccgaaacacgcgacctggcaaggatgagctctttgatctggaggagagtcacccacaggtggtgctggtggctgccaaccaccgctacgtctctgtgcggcaaggggtcaacgtctcagccaatcaggatgatgaactagaccacgagaccttcctgatgcaaattgaccaggagacaaagaagtgcaccttctattccagcactgggggctactggaccctggtcacccatgggggcattcacgccacagccacacaagtttctgccaacaccatgtttgagatggagtggcgtggccggcgggtagcactcaaagccagcaacgggcgctacgtgtgcatgaagaagaatgggcagctggcggctatcagcgattttgtcgggcccccaccccgcccggcctggacagggaaggtggcgggaggggcagcgcagcagacgctctccccgccaggcaaggacgaagagttcaccctcaagctcatcaaccggcccatcctggtgctgcgcggcctggacggcttcgtctgccaccaccgcggctccaaccagctggacaccaaccgctccgtctacgacgtcttccacctgagcttcagcgacggcgcctaccggatccgaggccgcgacggagggttctggtacacgggcagccacggcagcgtgtgcagcgacggcgaacgcgccgaggacttcgtcttcgagttccgtgagcgcggccgcctggccatccgcgcccggagcggcaagtacctgcgcggcggcgcctcgggcctgctgcgggccgatgccgacgccccggccgggaccgcgctttgggagtactgaggccgcgcccagaccagcctgtcgcgcattaaaaccgtgtctctcccgcaaaaaaaaaaaaaaaaaaaaa

SEQ ID NO: 5 - protein, 498 aa, Homo sapiens
MDETEWIHRHPKAEDLRVGLISWAGTYLTFEACKNTVTATAKSLGRRQTWEILVSNEHETQAVVRLKSVQGLYLLCECDGTVCYGRPRTSHHGCFLLRFHRNSKWTLQCLISGRYLESNGKDVFCTSHVLSAYHMWTPRPALHVHVILYSPIHRCYARADPTMGRIWVDAAVPCLEECGFLLHFRDGCYHLETSTHHFLSHVDRLFSQPSSQTAFHMQVRPGGLVALCDGEGGMLYPQGTHLLLGMGCNPMRGEEWFILQHCPTWVSLRSKTGRFISVIYDGEVRAASERLNRMSLFQFECDSESPTVQLRSANGYYLSQRRHRAVMADGHPLESDTFFRMHWNCGRIILQSCRGRFLGIAPNSLLMANVILPGPNEEFGILFANRSFLVLRGRYGYVGSSSGHDLIQCNQDQPDRIHLLPCRPGIYHFQAQGGSFWSITSFGTFRPWGKFALNFCIELQGSNLLTVLAPNGFYMRADQSGTLLADSEDITRECIWEF

SEQ ID NO: 6 - DNA, 1883 bp, Homo sapiens
ccctttccccactgtggtgtgataagaggctgccctcacagtcacaatgctcccgggtcacagaggtgctgggccccaggccagcctctgcctgggaagttctctctgggaacatctggtgggtactacaggccctattccaggccctatggcctgtggaacctcaccacgggggggagggctgggccagacggagacatcacctgtggtgtcagccccatggatgagacagagtggatacacagacatcccaaggctgaggacctaagggttgggctcatcagctgggcaggaacctacctcacctttgaggcatgcaagaatacagtcactgcaactgcgaagagtttgggcaggagacagacctgggagatcttggtgagcaatgagcatgagacacaggccgtggtgcgactaaagagcgtgcagggcctctacctgctgtgtgagtgtgatggcaccgtgtgttatggccgcccaaggaccagccaccatgggtgctttctactgcgtttccaccggaacagcaagtggaccctccagtgcctaatctctggtcgttatttggagtccaatggcaaggacgtgttttgcacttcccacgtcctctcagcttaccacatgtggaccccccgaccagccctccatgtccacgtgatcctctacagccccatccaccgctgctatgcccgggctgaccccactatgggccgcatctgggtggacgcagcagttccctgcctggaggagtgtggcttcctgttgcatttccgagatggatgctaccacctggagacctctacacaccacttcttgtcccatgtagaccggctgttctcccaaccctcatcacagacagcttttcacatgcaagtgcggcctggagggcttgtggcactgtgtgatggagaaggaggcatgttatatccacagggcacgcatctgctcttgggcatgggctgcaaccccatgaggggtgaggagtggttcatcctacagcactgcccaacctgggtcagcctcaggtcaaagactgggcggttcatctcagtcatctacgatggtgaggtgcgtgctgcttctgagcgcttaaaccgaatgtccttgttccagtttgaatgtgacagtgagagccccactgtgcagcttcgttcagccaatggctactacctatcccagaggcgccacagggcagtaatggctgatgggcaccccctggagtctgacacgttcttccgaatgcactggaactgtggcaggatcatcctgcagtcctgcagggggcgcttcctgggcattgcacccaacagcctgctgatggccaatgtcatccttccaggcccaaatgaggaatttgggattttatttgccaatcgctccttccttgtattgcgaggtcgttatggctatgtgggctcctcatcgggccatgacctcatacagtgcaaccaggatcagcccgaccgcattcatctactaccctgccgaccgggtatctaccacttccaggcacaggggggatccttctggtcaataacatcctttggcacctttcgcccttggggcaagtttgccctcaacttctgtatcgagcttcaggggagcaacttactcactgtactggcccccaatggcttctacatgcgagccgaccaaagtggcaccctgttggcagacagtgaagacattaccagagagtgtatctgggaattttaggtcaatgggatgtcacctaccaaaatccaaatcctccaggaaaaactactacactaaatggaccaggaacctcagagtcaagatccaagagaagaacatctgttacaacttttcctacccagtttagcaaaacacctgttttatgcaacaatacatcacaacaggcc

SEQ ID NO: 7 - protein, 493 aa, Mus musculus
MTANGTAEAVQIQFGLISCGNKYLTAEAFGFKVNASASSLKKKQIWTLEQPPDEAGSAAVCLRSHLGRYLAADKDGNVTCEREVPDGDCRFLVVAHDDGRWSLQSEAHRRYFGGTEDRLSCFAQSVSPAEKWSVHIAMHPQVNIYSVTRKRYAHLSARPADEIAVDRDVPWGVDSLITLAFQDQRYSVQTSDHRFLRHDGRLVARPEPATGFTLEFRSGKVAFRDCEGRYLAPSGPSGTLKAGKATKVGKDELFALEQSCAQVVLQAANERNVSTRQGMDLSANQDEETDQETFQLEIDRDTRKCAFRTHTGKYWTLTATGGVQSTASTKNASCYFDIEWCDRRITLRASNGKFVTAKKNGQLAASVETAGDSELFLMKLINRPIIVFRGEHGFIGCRKVTGTLDANRSSYDVFQLEFNDGAYNIKDSTGKYWTVGSDSSVTSSSDTPVDFFLEFCDYNKVALKVGGRYLKGDHAGVLKACAETIDPASLWEY

SEQ ID NO: 8 - DNA, 2667 bp, Mus musculus
tgtggagaactgcagcgggctaagccgtgttgaacaaaggaggtcgggcacagctatccaagctcccggggccaccgggccgccctccgccaccatgaccgccaacggcacggcagaggctgtgcagattcagttcgggctcatcagctgcggcaacaagtacctgacagccgaggcgttcgggttcaaggtgaacgcatccgctagtagcttgaaaaagaagcagatctggacgctggagcaacctcccgatgaggcgggcagcgcggccgtgtgtctgcgcagccacctgggtcgctacctggccgccgacaaggacggcaacgtgacctgcgagcgcgaggtgcccgacggcgactgccgctttctcgtcgtggcgcacgacgacggccgctggtcgctgcagtccgaggctcaccggcgctactttggcggcaccgaggaccgcctgtcctgcttcgcgcagagcgtgtcgccggccgagaagtggagcgtgcacatcgccatgcacccgcaggttaacatctacagcgttacccgcaagcgctacgcgcatctgagcgcgcggccggccgacgagatcgcggtagaccgcgacgtgccttggggcgtcgactcgctcatcaccttggccttccaggaccaacgctacagtgtgcagacgtccgaccaccgcttcctgcgccacgacgggcgccttgtggcacggccggagcccgccacgggcttcacgctggagttccgctccggcaaggtggcctttcgcgactgcgaaggtcgctacctggctccgtccgggcccagcggcaccctcaaggctggcaaggccaccaaggtgggcaaagatgagctcttcgccctggaacagagctgcgctcaggtggtgctgcaggcggccaacgagaggaacgtgtccacgcgccagggaatggacctgtcagccaatcaggatgaagagaccgatcaggagaccttccagctggagatcgaccgcgacacaagaaagtgtgcctttcgcacccacacgggcaagtactggacactgacggcgaccggaggtgtgcaatccactgcgtccaccaagaacgccagctgctactttgacatcgagtggtgtgaccgccggatcactctgagagcctccaacggcaagtttgtgaccgccaagaaaaatggccagctggccgcctcggtggagacagcaggggactcggaactcttcctcatgaagctgattaaccgccccatcatcgtgttccggggggaacacgggttcattggctgccgcaaggtcacgggcactctggatgccaaccgttccagttacgatgtcttccagttggaattcaatgacggcgcctacaacatcaaagactccacgggcaagtactggacggtgggtagtgattcctcggtcaccagcagcagcgacacccctgtggatttcttccttgagttctgtgactacaataaggtggctctcaaggtgggcggccgctacctgaagggggaccacgctggggtcctgaaggcctgcgcggagactatcgaccccgcctcactctgggagtactagggccacctgccctctgcaggccgctctcgtcagtccctcctgttatccttactcatcgggtggccctgcagcaggtggcaaaccccttgcctttcaaactggaaacccaagagaaaacggtgcccttgctgtcaccctctgtggaccccttttccctaactcactgctccccatgggtcggtggctgcagactgtccccaggagggactctggttccctctgtccccttctttccatggggaactctggcacctttcttctgacctcagtcaactctgagccttatttccccccaggaagtggcctaggagaagctacagggcctagggacttaccctgagcttgtaactggaagaccccgtccctatccccgctcccgcccccaccccaccccacccctgctctggccccagcctctggaggccagccttttggcgggactgaagccgggcatggccaaccttgcccacaagtgtttttctggatcttggctggaaggcagtctgtcccatcctgcagtgtttgggcctggctctttgactcaaagctagctaggtggcactccgtgtcgctcctgcacattctggaaggggcgggcctctcacccacctcattccttttccccctggcctgactggaagcagaaaaatgaccaaatcagtatttttttttttttctttaaggaaatgttactgttgaaaggccctaggcaagcctgccctgttggttgtagtcgtgagtggtcttggggggagatgcttggctcctgtccctgcctccccagcgggttccctccctccctcctgcctgaccaccccagctctggctctgtgattggtgctccacgtcttcccagacacctcggggctcctgggcgggagaaagccggatgtgcccctccctgggagccctggagtaaacctcagggggccctttcccaatcacccccttccaccgacccctcaacaccatgcatctcactctgggtgtctcgctcctttatttttttgtaactgtcatttctataactctgaagacccatgatagtaagctttgaactggaaaataaagtaaaatcaagtctgcggccc

SEQ ID NO: 9 - protein, 235 aa, Homo sapiens
SCAQVVLQAANERNVSTRQGMDLSANQDEETDQETFQLEIDRDTKKCAFRTHTGKYWTLTATGGVQSTASSKNASCYFDIEWRDRRITLRASNGKFVTSKKNGQLAASVETAGDSELFLMKLINRPIIVFRGEHGFIGCRKVTGTLDANRSSYDVFQLEFNDGAYNIKDSTGKYWTVGSDSAVTSSGDTPVDFFFEFCDYNKVAIKVGGRYLKGDHAGVLKASAETVDPASLWEY

SEQ ID NO: 10 - protein, 258 aa, Homo sapiens
PQVVLVAANHRYVSVRQGVNVSANQDDELDHETFLMQIDQETKKCTFYSSTGGYWTLVTHGGIHATATQVSANTMFEMEWRGRRVALKASNGRYVCMKKNGQLAAISDFVGPPPRPAWTGKVAGGAAQQTLSPPGKDEEFTLKLINRPILVLRGLDGFVCHHRGSNQLDTNRSVYDVFHLSFSDGAYRIRGRDGGFWYTGSHGSVCSDGERAEDFVFEFRERGRLAIRARSGKYLRGGASGLLRADADAPAGTALWEY

SEQ ID NO: 11 - protein, 240 aa, Homo sapiens
LQHCPTWVSLRSKTGRFISVIYDGEVRAASERLNRMSLFQFECDSESPTVQLRSANGYYLSQRRHRAVMADGHPLESDTFFRMHWNCGRIILQSCRGRFLGIAPNSLLMANVILPGPNEEFGILFANRSFLVLRGRYGYVGSSSGHDLIQCNQDQPDRIHLLPCRPGIYHFQAQGGSFWSITSFGTFRPWGKFALNFCIELQGSNLLTVLAPNGFYMRADQSGTLLADSEDITRECIWEF

SEQ ID NO: 12 - protein, 235 aa, Mus musculus
SCAQVVLQAANERNVSTRQGMDLSANQDEETDQETFQLEIDRDTRKCAFRTHTGKYWTLTATGGVQSTASTKNASCYFDIEWCDRRITLRASNGKFVTAKKNGQLAASVETAGDSELFLMKLINRPIIVFRGEHGFIGCRKVTGTLDANRSSYDVFQLEFNDGAYNIKDSTGKYWTVGSDSSVTSSSDTPVDFFLEFCDYNKVALKVGGRYLKGDHAGVLKACAETIDPASLWEY

SEQ ID NO: 13 - DNA, 19 bp, Homo sapiens
ccagcagcctgaagaagaa

SEQ ID NO: 14 - DNA, 19 bp, Homo sapiens
ggcaagtactggacgctga

SEQ ID NO: 15 - DNA, 19 bp, Homo sapiens
caaagactccacaggcaaa

SEQ ID NO: 16 - DNA, 19 bp, Homo sapiens
ataacaaggtggccatcaa

SEQ ID NO: 17 - DNA, 19 bp, Homo sapiens
ctgaaggcctcggcggaaa

SEQ ID NO: 18 - DNA, 19 bp, Homo sapiens
tcaaagactccacaggcaa

SEQ ID NO: 19 - DNA, 19 bp, Homo sapiens
agaccgaccaggagacctt

SEQ ID NO: 20 - DNA, 19 bp, Homo sapiens
ctgaagaagaagcagatct

SEQ ID NO: 21 - DNA, 19 bp, Homo sapiens
ccttccaggaccagcgcta

SEQ ID NO: 22 - DNA, 19 bp, Homo sapiens
ccaaggtgggcaaggacga

SEQ ID NO: 23 - DNA, 19 bp, Homo sapiens
actataacaaggtggccat

SEQ ID NO: 24 - DNA, 19 bp, Homo sapiens
gcgttcgggttcaaggtga

SEQ ID NO: 25 - DNA, 19 bp, Homo sapiens
gccagcagcctgaagaaga

SEQ ID NO: 26 - DNA, 19 bp, Homo sapiens
aagaagaagcagatctgga

SEQ ID NO: 27 - DNA, 19 bp, Homo sapiens
gtcaacatctacagcgtca

SEQ ID NO: 28 - DNA, 19 bp, Homo sapiens
gtatggacctgtctgccaa

SEQ ID NO: 29 - DNA, 19 bp, Homo sapiens
gcgcctacaacatcaaaga

SEQ ID NO: 30 - DNA, 19 bp, Homo sapiens
gcgacactcctgtggactt

SEQ ID NO: 31 - DNA, 19 bp, Homo sapiens
ccgccagcagcctgaagaa

SEQ ID NO: 32 - DNA, 19 bp, Homo sapiens
agaagtggagcgtgcacat

SEQ ID NO: 33 - DNA, 19 bp, Homo sapiens
tgggcaaggacgagctctt

SEQ ID NO: 34 - DNA, 19 bp, Homo sapiens
gcctgaagaagaagcagat

SEQ ID NO: 35 - DNA, 19 bp, Homo sapiens
ccaatcaggacgaggagac

SEQ ID NO: 36 - DNA, 19 bp, Homo sapiens
gaggagaccgaccaggaga

SEQ ID NO: 37 - DNA, 19 bp, Homo sapiens
agatcgaccgcgacaccaa

SEQ ID NO: 38 - DNA, 19 bp, Homo sapiens
gagcatggcttcatcggct

SEQ ID NO: 39 - DNA, 19 bp, Homo sapiens
tgtccacgcgccagggtat

SEQ ID NO: 40 - DNA, 19 bp, Homo sapiens
gctgctactttgacatcga

SEQ ID NO: 41 - DNA, 19 bp, Homo sapiens
ggcaaatactggacggtgg

SEQ ID NO: 42 - DNA, 19 bp, Homo sapiens
agcctgaagaagaagcaga

SEQ ID NO: 43 - DNA, 19 bp, Homo sapiens
ggtatggacctgtctgcca

SEQ ID NO: 44 - DNA, 19 bp, Homo sapiens
ctgccaatcaggacgagga

SEQ ID NO: 45 - DNA, 19 bp, Homo sapiens
tttgtgacctccaagaaga

SEQ ID NO: 46 - DNA, 19 bp, Homo sapiens
acgccaaccgctccagcta

SEQ ID NO: 47 - DNA, 19 bp, Homo sapiens
cctacaacatcaaagactc

SEQ ID NO: 48 - DNA, 19 bp, Homo sapiens
atcaaagactccacaggca

SEQ ID NO: 49 - DNA, 19 bp, Homo sapiens
agttctgcgactataacaa

SEQ ID NO: 50 - DNA, 19 bp, Homo sapiens
gacaaggacggcaacgtga

SEQ ID NO: 51 - DNA, 19 bp, Homo sapiens
aggtcaacatctacagcgt

SEQ ID NO: 52 - DNA, 19 bp, Homo sapiens
acgaggagaccgaccagga

SEQ ID NO: 53 - DNA, 19 bp, Homo sapiens
gcaagtttgtgacctccaa

SEQ ID NO: 54 - DNA, 19 bp, Homo sapiens
tgaagaagaagcagatctg

SEQ ID NO: 55 - DNA, 19 bp, Homo sapiens
tcgagttctgcgactataa

SEQ ID NO: 56 - DNA, 19 bp, Homo sapiens
agatctggacgctggagca

SEQ ID NO: 57 - DNA, 19 bp, Homo sapiens
gcctacaacatcaaagact

SEQ ID NO: 58 - DNA, 19 bp, Homo sapiens
cttcgagttctgcgactat

SEQ ID NO: 59 - DNA, 19 bp, Homo sapiens
accctcaggtcaacatcta

SEQ ID NO: 60 - DNA, 19 bp, Homo sapiens
tcaactgcggcaacaagta

SEQ ID NO: 61 - DNA, 19 bp, Homo sapiens
ctggagatcgaccgcgaca

SEQ ID NO: 62 - DNA, 19 bp, Homo sapiens
cctccaagaagaatgggca

SEQ ID NO: 63 - DNA, 19 bp, Mus musculus
ggtgggcaaagatgagctc

SEQ ID NO: 64 - DNA, 19 bp, Mus musculus
gtggagcgtgcacatcgcc

SEQ ID NO: 65 - DNA, 19 bp, Homo sapiens
ggtgggcaaggacgagctc

SEQ ID NO: 66 - DNA, 19 bp, Homo sapiens
gcctgaagaagaagcagat